(19) United States
(12) Patent Application Publication
(10) Pub. No.: US 2026/0285897 A1
(43) Pub. Date: Sep. 24, 2026

(54) Neurosteroids and Prodrugs Thereof

(71) Applicant: Emory University, Atlanta, GA (US)

(72) Inventors: Dennis C. Liotta, Atlanta, GA (US); Gouthami Pashikanti, Atlanta, GA (US); Luke E. Hodson, Atlanta, GA (US); Alet E. van der Westhuyzen, Atlanta, GA (US); Russell G. Fritzemeier, Atlanta, GA (US); Jiafeng Geng, Atlanta, GA (US)

(21) Appl. No.: 19/168,284

(22) PCT Filed: Mar. 25, 2024
(86) PCT No.: PCT/US2024/021381

§ 371 Date: Sep. 23, 2025

Related U.S. Application Data
(60) Provisional application No. 63/491,749, filed on Mar. 23, 2023.

(51) Int. Cl. C07J 7/00; A61K 31/57; A61K 31/58; C07J 41/00; C07J 43/00

(52) U.S. Cl. CPC C07J 7/002; A61K 31/57; A61K 31/58; C07J 41/005; C07J 43/003

(57) ABSTRACT

Disclosed are neurosteroids and prodrugs thereof. Pharmaceutical formulations containing the neurosteroids and prodrugs are also disclosed. Additionally, methods of treating a condition, disorder, or disease using the neurosteroids, prodrugs, or pharmaceutical formulations are disclosed. Exemplary conditions, disorders, and diseases relevant to this disclosure include stroke, subarachnoid hemorrhage, cerebral ischemia, cerebral vasospasm, hypoxia, CNS injury, concussion, traumatic brain injury, depression, postpartum depression, epilepsy, seizure disorder, essential tremor, fragile X syndrome, and neurodegenerative disease.

### CROSS-REFERENCE TO RELATED APPLICATIONS

This application claims the benefit of U.S. Provisional Application No. 63/491,749 filed Mar. 23, 2023. The entirety of this application is hereby incorporated by reference for all purposes.

### TECHNICAL FIELD

The present disclosure relates to neurosteroids and prodrugs thereof. It also relates to pharmaceutical formulations of the neurosteroids and prodrugs as well as methods for treating conditions, disorders, or diseases using the neurosteroids and prodrugs.

### BACKGROUND

In recent years, there has been a growing body of experimental evidence demonstrating that neurosteroids are effective neuroprotective and/or neuromodulating agents. Generally, neurosteroids are steroids that can be synthesized in the CNS independent of endocrine sources and display neuroactive effects. Neurosteroids have anti-inflammatory, antioxidant, and/or neuroprotective roles and engage various neurological targets such as ligand-gated ion channels and other cell surface receptors, including GABA receptors and glutamate receptors (e.g., NMDA receptors), among others (Tsutsui K, Haraguchi S, Handbook of Hormones, 2016, 537).

Some major known biological functions of neurosteroids include modulation of neural plasticity (Benarroch E E, Neurology, 2007, 68(12):945-7), learning and memory processes (Valle M, Mayo W, Koob G F, Le Moal M, International Review of Neurobiology, 2001 46:273-320), behavior (Engel S R, Grant K A, International Review of Neurobiology, 2001, 46:321-48), seizure susceptibility (Joshi S, Rajasekaran K, Kapur J, Experimental Neurology, 2013, 244:36-42), as well as responses to stress, anxiety, and depression (Frye C A, Psychoneuroendocrinology, 2009, 34). For example, acute stress elevates the levels of inhibitory neurosteroids like allopregnanolone, and these neurosteroids are known to counteract many of the effects of stress (Bali A, Jaggi A S, Progress in Neuro-Psychopharmacology & Biological Psychiatry, 2014, 48:64-78). Additionally, chronic stress has been associated with diminished levels of allopregnanolone and altered allopregnanolone stress responsivity, psychiatric disorders, and hypothalamic-pituitary-adrenal axis dysregulation (Girdler S S, Klatzkin R, Pharmacology & Therapeutics, 2007, 116(1):125-39).

Further, fluctuations in the levels of inhibitory neurosteroids during the menstrual cycle and pregnancy play an important role in a variety of women's conditions, including premenstrual syndrome (PMS), premenstrual dysphoric disorder (PMDD), postpartum depression (PPD), postpartum psychosis, and catamenial epilepsy (Backstrom T, et al., Annals of the New York Academy of Sciences, 2003, 1007(1):42-53; Guille C, Spencer S, Cavus I, Epperson C N, Epilepsy & Behavior, 2008, 13(1):12-24; Finocchi C, Ferrari M, Neurological Sciences, 2011, 32).

Neurosteroids have a wide range of clinical applications from sedation to treatment of epilepsy (Reddy D S, Rogawski M A, Neurotherapeutics, 2009, 6(2):392-401), traumatic brain injury and stroke (Morrow A L, Pharmacology & Therapeutics, 2007, 116(1):1-6; Dubrovsky B O, Progress in Neuro-Psychopharmacology & Biological Psychiatry, 2005, 29(2):169-92), neurodegenerative diseases such as dementia and Alzheimer's disease (Hernandez G, et al., Neurology, 2022, 98), depression such as major depressive disorder and postpartum depression (Almeida F B, Nin, MS, Barros H M T, Neurobiol Stress, 2020, 12:100218), PTSD (Pinna G, Front. Endocrinol., 2020, 11, 236), and fragile X syndrome (Wang J Y, et al., Neurotherapeutics, 2017, 4(4):1073-1083). Additionally, preclinical and clinical research has shown that neurosteroids such as allopregnanolone, epipregnanolone, pregnenolone, and dehydroepiandrosterone can dramatically reduce cerebral edema, inflammation, tissue necrosis, and programmed cell death (Garcia-Estrada, et al., International Journal of Developmental Neuroscience, 1999, 17, 145-161; Djebaili, et al., J Neurotrauma, 2005, 22, 106-118; Pettus, et al., Brain Res, 2005, 1049, 112-119; Grossman, et al., Brain Res, 2004, 1008, 29-39; He, et al., Exp Neurol, 2004, 189, 404-412; Balan, et al., Scientific Reports, 2019, 9, 1220).

Allopregnanolone is a positive allosteric modulator of the GABA_{A} receptor, whose activities influence an array of behavioral states, such as anxiety levels, seizures, sleep, vigilance, and memory. As a result, allopregnanolone has numerous therapeutic effects, including antidepressant, cognitive enhancement, antiepileptic, neuroprotective, and neurogenic effects. It has been or is being evaluated in many clinical trials for treating neurodegenerative diseases (such as Alzheimer's disease), depression and postpartum depression, PTSD, TBI, epilepsy, and addictions (such as alcohol abuse disorder). In 2019, allopregnanolone was approved by the FDA for treating postpartum depression. Ganaxolone is a medication used to treat seizures. It was approved by the FDA for treating seizures in people with cyclin-dependent kinase-like 5 deficiency disorder (CDD). Similar to allopregnanolone, ganaxolone binds to allosteric sites of the GABA_{A} receptor to modulate and open the chloride ion channel, resulting in a hyperpolarization of the neuron (Reddy D S, Rogawski M A, Epilepsy Research, 2010, 89: 254-60). Zuranolone and its analogs are under development for the treatment of depressive disorders and a variety of other indications (WO2022/232104 and WO2021/067089). Similar to ganaxolone, zuranolone is an orally active, inhibitory pregnane neurosteroid, acting as a positive allosteric modulator of the GABA_{A} receptor (Botella M G, et al., Journal of Medicinal Chemistry, 2017, 60(18):7810-7819; Blanco M J, et al., Bioorganic & Medicinal Chemistry Letters, 2018, 28(2):61-70).

Brain injuries, such as those caused by TBI and stroke, can trigger an inflammatory immune response and excitotoxicity resulting from disruption of the glutamate, acetylcholine, cholinergic, GABA, and/or NMDA receptor systems. As a result, cytokines are released and signal the delivery of bloodborne leukocytes to the corresponding injury sites to neutralize potential pathogens and promote tissue repair. However, the powerful inflammatory response has the capacity to cause damages to normal tissue, leading to neuronal loss. In addition to brain injuries, inflammation is recognized as a key component of many central nervous system (CNS) disorders and diseases, such as neurodegenerative diseases, including dementia and Alzheimer's disease.

Brain injury treatments rely on symptom management with the goal of mitigating secondary injury due to inflammation and edema. Not surprisingly, minimizing the time from symptom onset to treatment is considered paramount in reducing the likelihood of long-term damage. Unfortunately, previous investigations into the use of neurosteroids for the treatment of brain injury typically required administration in a hospital setting, thus losing valuable time before the treatment could be administered. Unfortunately, most pregnane and androstane neurosteroids, are insoluble in aqueous-based formulations and require complicated and time-consuming lipid formulations that preclude use in a prehospital setting. Furthermore, the plasma half-life of neurosteroids is limited, and treatment requires prolonged intravenous infusion or multiple injections, which further delays treatment. The aforementioned flaws of neurosteroids are believed to contribute to the recent failure of two Phase III clinical trials on the use of progesterone for TBI treatment (Stein, Brain Inj, 2015, 19, 29, 1259-1272). Even for non-acute CNS disorders and diseases, such as depression, epilepsy, and neurodegenerative diseases, the aqueous insolubility of pregnane and androstane neurosteroids significantly limits their application in the clinical setting. For example, allopregnanolone is given as a 60-hr IV infusion for postpartum women in a healthcare facility; ganaxolone is given as an oral suspension for patients suffer from cyclin-dependent kinase-like 5 (CDKL5) deficiency disorder.

There is an urgent need for pharmacological treatments for CNS diseases, disorders, and conditions that improve short and long-term neurological outcomes. While existing neurosteroids have promising neuroprotective potentials, there is an urgent need for improved neurosteroids and prodrugs thereof that can be administered easily and rapidly for treating both acute and non-acute CNS diseases, disorders, and conditions.

### SUMMARY

The present disclosure describes neurosteroids and prodrugs of the neurosteroids.

Generally, the neurosteroids have a 20-carbon skeleton, as shown in Formula I. Notably, the neurosteroids contain an oxime group at the C20 position. This structure feature is different from many endogenous neurosteorids, which contain a ketone group at the C20 position. Installation of the oxime group eliminates ketone reduction, which is one of the metabolic pathways of endogenous neurosteorids, such as allopregnanolone, while retaining or even enhancing the biological activities, including modulation of the GABA receptors. For example, as the present inventors have shown, installation of the oxime group may selectively potentiate a GABA_{A} receptor containing the delta (6) subunit, compared to a GABA_{A} receptor without the delta (6) subunit.

Generally, the prodrugs have a higher solubility in an aqueous medium than their corresponding neurosteroid parent compounds. In some embodiments, the prodrugs are capable of self-immolative cleavage in response to environmental pH changes, releasing the corresponding neurosteroid parent compounds. In some embodiments, the prodrugs are stable in an acidic aqueous medium but exhibit a wide range of release kinetics in human plasma. In some embodiments, the prodrugs contain a phosphate group and are readily cleavable by phosphatases.

The neurosteroids have a structure of Formula I or a pharmaceutically acceptable salt, hydrate, or hydrated salt of Formula I,

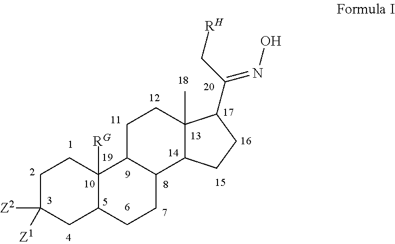

wherein R^{G} is hydrogen or methyl;
wherein R^{H} is hydrogen or an optionally substituted nitrogen-containing 5-membered heteroaryl;
wherein Z¹ is —OR^{X}, wherein R^{X} is selected from hydrogen, acyl, ester, thioester, and amide; and
wherein Z² is methyl, halogen-substituted methyl, methoxymethyl, or halogen-substituted methoxymethyl.

In some embodiments, Formula I is in the form of one of the following formulas.

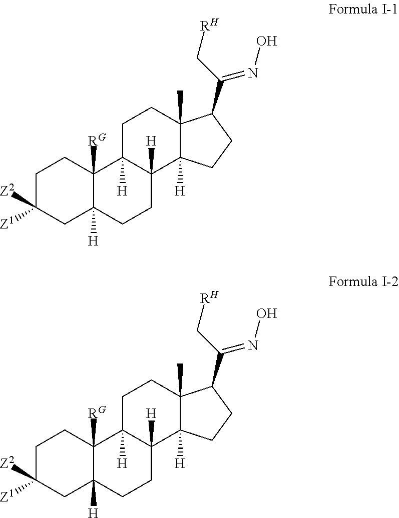

In some embodiments, R^{G} is hydrogen. In some embodiments, R^{G} is methyl.

In some embodiments, R^{H} is hydrogen. In some embodiments, R^{H} is optionally substituted nitrogen-containing 5-membered heteroaryl.

In some embodiments, Z¹ is —OH.

In some embodiments, Z² is selected from methyl, —CF₃, and —CH₂OCH₃.

The prodrugs of the neurosteroids may have a structure of Formula II or a pharmaceutically acceptable salt, hydrate, or hydrated salt of Formula II,

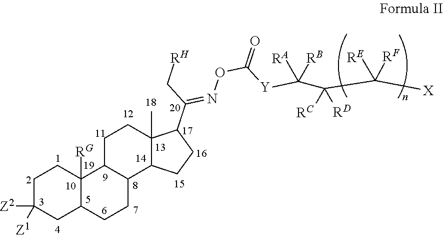

wherein R^{G}, R^{H}, Z¹, and Z² are the same as described in Formula I; and
wherein:
(1) X is OH or NR¹R²,
   Y is O or NR³,
   R^{A}, R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
   R¹, R², and R³ are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen;
(2) X is NR¹R²,
   Y is O or NR³,
   R¹ joins R^{C} or R^{E} to form a 4-7 membered, optionally substituted heterocycle,
   R² is hydrogen,
   R^{A}, R^{B}, R^{C}, R^{D}, R^{E}, and R^{F}, on each occurrence when not joined by R¹ to form the heterocycle, are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
   R³ is independently selected from hydrogen, optionally substituted alkyl, optionally substituted heteroalkyl, optionally substituted carbocyclyl, optionally substituted heterocyclyl, optionally substituted aryl, and optionally substituted heteroaryl; or
(3) X is OH or NR¹R²,
   Y is NR³,
   R³ joins R^{A} to form a 4-7 membered, optionally substituted heterocycle,
   R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
   R¹ and R² are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

In some embodiments, Formula II is in the form of one of the following formulas.

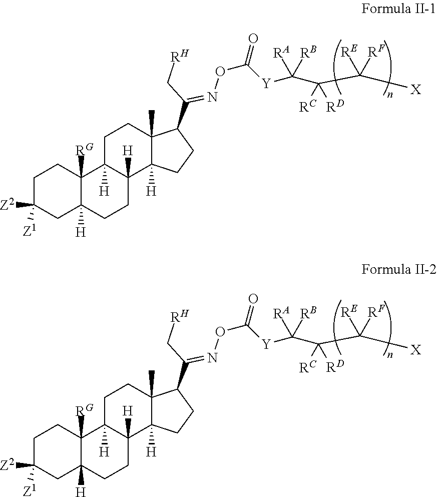

In some embodiments, n is 0. In some embodiments, n is 1.

Optionally, Formula II and its sub-formulas may have the following features:
X is OH or NR¹R²,
Y is O or NR³,
R^{A}, R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹, R², and R³ are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

In some embodiments, X is NR¹R². In some embodiments, R¹ is an optionally substituted C₁-C₄ alkyl, such as —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, or —CH₂CH₂N(CH₃)₃₄.

In some embodiments, Y is NR³. In some embodiments, R³ is an optionally substituted C₁-C₄ alkyl, such as —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, or —CH₂CH₂N(CH₃)₃⁺. In some embodiments, R³ is optionally substituted carbocyclyl or optionally substituted heterocyclyl, such as

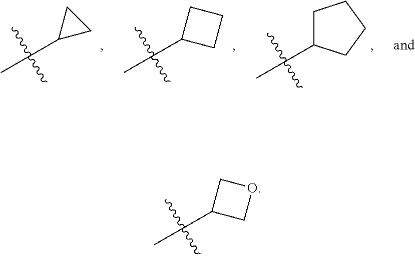

In some embodiments, both R^{A} and R^{B} are hydrogen. In some embodiments, both R^{C} and R^{D} are hydrogen. In some embodiments, when present, both R^{E} and R^{F} are hydrogen.

In some embodiments, n is 0, X is NR¹R², and Y is NR³.

Optionally, Formula II and its sub-formulas may have the following features:
X is NR¹R²,
Y is O or NR³,
R¹ joins R^{C} or R^{E} to form a 4-7 membered, optionally substituted heterocycle,
R² is hydrogen,
R^{A}, R^{B}, R^{C}, R^{D}, R^{E}, and R^{F}, on each occurrence when not joined by R¹ to form the heterocycle, are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R³ is independently selected from hydrogen, optionally substituted alkyl, optionally substituted heteroalkyl, optionally substituted carbocyclyl, optionally substituted heterocyclyl, optionally substituted aryl, and optionally substituted heteroaryl.

In some embodiments, the 4-7 membered, optionally substituted heterocycle is a 5 or 6 membered, optionally substituted heterocycle, such as an optionally substituted pyrrolidine or optionally substituted piperidine.

In some embodiments, Y is NR³. In some embodiments, R³ is an optionally substituted C₁-C₄ alkyl, such as —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, or —CH₂CH₂N(CH₃)₃⁺. In some embodiments, R³ is optionally substituted carbocyclyl or optionally substituted heterocyclyl, such

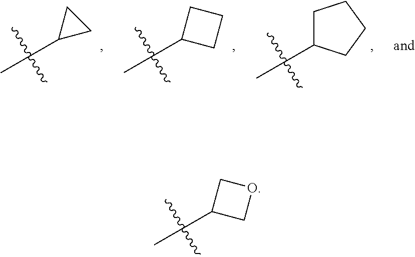

In some embodiments, both R^{A} and R^{B} are hydrogen. In some embodiments, R^{D} is hydrogen. In some embodiments, when present, R^{F} is hydrogen.

In some embodiments, n is 0, and Y is NR³.

In some embodiments, n is 1, and Y is NR³. In some embodiments, n is 1, Y is NR³, and R¹ joins R^{E} to form the 4-7 membered, optionally substituted heterocycle.

The prodrugs of the neurosteroids may have a structure of Formula III or a pharmaceutically acceptable salt, hydrate, or hydrated salt of Formula III,

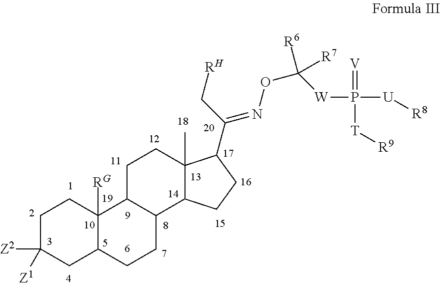

wherein R^{G}, R^{H}, Z¹, and Z² are the same as described in Formula I;
wherein T, U, V, and W are independently O or S;
wherein R⁶ and R⁷ are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
wherein R¹ and R⁹ are independently selected from hydrogen, optionally substituted alkyl, optionally substituted heteroalkyl, optionally substituted carbocyclyl, optionally substituted heterocyclyl, optionally substituted aryl, and optionally substituted heteroaryl.

In some embodiments, Formula III is in the form of one of the following formulas.

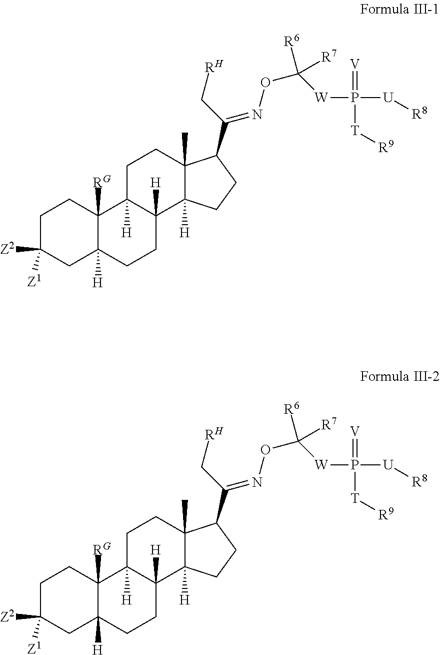

Optionally, Formula III and its sub-formulas may have the following features:
T, U, V, and W are O, and
R⁶, R⁷, R⁸, and R⁹ are hydrogen.

This disclosure also provides deuterated analogs of (1) the neurosteroids and (2) the prodrugs of the neurosteroids, wherein one or more non-ionizable hydrogen atoms in the

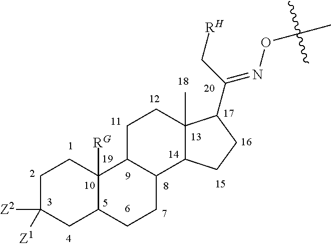

moiety of (1) the neurosteroids and (2) the prodrugs of the neurosteroids are replaced with deuterium. In some embodiments, the hydrogen atom at the C17 position is replaced with deuterium. In some embodiments, the hydrogen atom at the C5 position is replaced with deuterium. In some embodiments, the two hydrogen atoms at the C4 position are replaced with deuterium. In some embodiments, the two hydrogen atoms at the C6 position are replaced with deuterium. In some embodiments, the two hydrogen atoms of the methylene group at the C20 position are replaced with deuterium. In some embodiments, when R^{H} is hydrogen, the corresponding methyl group at the C20 position is fully deuterated. In some embodiments, Z² is deuterated, either fully deuterated or partially deuterated.

Also disclosed are compositions containing a compound described herein, wherein the compound is in greater than 80%, 85%, 90%, or 95% enantiomeric or diastereomeric excess. In this context, the compound may be a neurosteroid described herein, a prodrug described herein, or a deuterated analog described herein. In some embodiments, the compound in the compositions is in greater than 95% enantiomeric or diastereomeric excess.

Also disclosed are pharmaceutical formulations of the disclosed compounds or compositions. In general, the pharmaceutical formulations contain a pharmaceutically acceptable excipient. In some embodiments, the pharmaceutical formulations are in a form chosen from tablets, capsules, caplets, pills, beads, granules, particles, powders, gels, creams, solutions, suspensions, emulsions, and nanoparticulate formulations. In some embodiments, the pharmaceutical formulations are oral formulations. In some embodiments, the pharmaceutical formulations are intravenous formulations. In some embodiments, the pharmaceutical formulations are intramuscular formulations. In some embodiments, the pharmaceutical formulations are in the form of solutions, such as aqueous solutions. In some embodiments, the pharmaceutical formulations are in the form of powders, such as lyophilized powders. In some embodiments, the pharmaceutical formulations are in the form of tablets, caplets, capsules, or pills.

This disclosure also relates to (1) the compounds, compositions, and pharmaceutical formulations disclosed herein for treatment of a condition, disorder, or disease disclosed herein or use as a medicament, (2) the compounds, compositions, and pharmaceutical formulations disclosed herein for use in the treatment of a condition, disorder, or disease disclosed herein, or (3) the compounds, compositions, and pharmaceutical formulations disclosed herein for the manufacture of a medicament for treatment of a condition, disorder, or disease disclosed herein.

This disclosure also provides methods of treating a condition, disorder, or disease in a subject in need thereof. The method includes administering an effective amount of a compound, composition, or pharmaceutical formulation disclosed herein to the subject. In some embodiments, the compound, composition, or pharmaceutical formulation is administered orally, intravenously, or intramuscularly.

Exemplary conditions, disorders, and diseases relevant to this disclosure include, but are not limited to, stroke, subarachnoid hemorrhage, cerebral ischemia, cerebral vasospasm, hypoxia, CNS injury, concussion, traumatic brain injury, depression, postpartum depression, epilepsy, seizure disorder, essential tremor, fragile X syndrome, and neurodegenerative disease.

### BRIEF DESCRIPTION OF DRAWINGS

FIG. 1 illustrates the decomposition of non-phosphate prodrugs in response to environmental pH changes.

FIG. 2 illustrates the release of compound 10 (C20-oxime analog of ganaxolone) over time (hr) from prodrug 22 (POM phosphate prodrug of C20-oxime analog of ganaxolone) in mouse plasma and brain after IV dosing of prodrug 22 (16.7 mg/kg) to C57BL/6 mice.

FIG. 3 illustrates the release of compound 10 (C20-oxime analog of ganaxolone) over time (hr) from prodrug 22 (POM phosphate prodrug of C20-oxime analog of ganaxolone) in mouse plasma and brain after oral dosing of prodrug 22 (33.4 mg/kg) to C57BL/6 mice.

### DETAILED DESCRIPTION

The present disclosure describes neurosteroids and prodrugs of the neurosteroids. Also disclosed are deuterated or halogenated analogs of (1) the neurosteroids and (2) the prodrugs of the neurosteroids.

In general, the neurosteroids have a 20-carbon skeleton, as shown in Formula I. Notably, the neurosteroids contain an oxime group at the C20 position. This structure feature is different from many endogenous neurosteorids, which contain a ketone group at the C20 position.

Installation of the oxime group eliminates ketone reduction, which is one of the metabolic pathways of endogenous neurosteorids, such as allopregnanolone, while retaining or even enhancing the biological activities, including modulation of the GABA receptors. For example, as the present inventors have shown, installation of the oxime group may selectively potentiate a GABA_{A} receptor containing the delta (δ) subunit, compared to a GABA_{A} receptor without the delta (δ) subunit. Notably, the delta (δ) subunit of the GABA_{A} receptor plays a critical role in neurological disorders such as depression, especially postpartum depression, and anxiety.

In general, the prodrugs have a higher solubility in an aqueous medium than their corresponding neurosteroid parent compounds. In some embodiments, the prodrugs are capable of self-immolative cleavage in response to environmental pH changes, releasing the corresponding neurosteroid parent compounds. In some embodiments, the prodrugs are stable in an acidic aqueous medium but exhibit a wide range of release kinetics in human plasma. In some embodiments, the prodrugs contain a phosphate group and are readily cleavable by phosphatases.

Also disclosed are pharmaceutical formulations containing a compound described herein and methods for treating conditions, disorders, or diseases using a compound described herein. In this context, the compound may be a neurosteroid described herein, a prodrug described herein, or a deuterated or halogenated analog described herein. Exemplary conditions, disorders, and diseases relevant to this disclosure include stroke, subarachnoid hemorrhage, cerebral ischemia, cerebral vasospasm, hypoxia, CNS injury, concussion, traumatic brain injury, depression, postpartum depression, epilepsy, seizure disorder, essential tremor, fragile X syndrome, and neurodegenerative disease.

Before the present disclosure is described in greater detail, it is to be understood that this disclosure is not limited to the particular embodiments described herein, and as such, may vary in accordance with the scope of the present disclosure. Unless defined otherwise, all technical and scientific terms used herein have the same meaning as commonly understood by one of ordinary skill in the art to which this disclosure belongs.

All publications and patents cited in this specification are herein incorporated by reference as if each individual publication and patent were specifically and individually indicated to be incorporated by reference. They are incorporated by reference to disclose and describe the methods and/or materials in connection with which the publications and patents are cited.

As will be apparent to those of ordinary skill in the art upon reading this disclosure, each of the particular embodiments described and illustrated herein has discrete components and/or features that may be readily separated from or combined with one or more components and/or features of any of the other embodiments described herein, without departing from the scope or spirit of the present disclosure. Any recited method can be carried out in the order of events recited herein or in any other order that is logically possible.

Embodiments of the present disclosure will employ, unless otherwise indicated, techniques of medicine, organic chemistry, medicinal chemistry, biochemistry, molecular biology, pharmacology, neurology, and the like, which are within the skill of the art. Such techniques are explained fully in the literature, such as the publications and patents cited herein.

### I. Definitions

As used herein, the singular forms “a,” “an,” and “the” include plural referents unless the context clearly dictates otherwise.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present as well as instances where it does not occur or is not present.

Use of the term “about” is intended to describe values either above or below the stated value in a range of approx. +/−10%; in other examples the values may range in value either above or below the stated value in a range of approx. +/−5%; in other examples the values may range in value either above or below the stated value in a range of approx. +/−2%; in other examples the values may range in value either above or below the stated value in a range of approx. +/−1%.

A carbon range (e.g., C₁-C₁₀) is intended to disclose individually every possible carbon value and/or sub-range encompassed within. For example, a carbon range of C₁-C₁₀ discloses C₁, C₂, C₃, C₄, C₅, C₆, C₇, C₈, C₉, and C₁₀, as well as sub-ranges encompassed therein, such as C₂-C₉, C₃-C₈, C₁-C₅, etc.

As used herein, the term “subject” refers to an animal, including human and non-human animals. Human subjects may include pediatric patients and adult patients. Non-human animals may include domestic pets, livestock and farm animals, and zoo animals. In some cases, the non-human animals may be non-human primates.

As used herein, the terms “prevent” and “preventing” include the prevention of the occurrence, onset, spread, and/or recurrence. It is not intended that the present disclosure is limited to complete prevention. For example, prevention is considered as achieved when the occurrence is delayed, the severity of the onset is reduced, or both.

As used herein, the terms “treat” and “treating” include medical management of a condition, disorder, or disease of a subject as would be understood by a person of ordinary skill in the art (see, for example, Stedman's Medical Dictionary). In general, treatment is not limited to cases where the subject is cured and the condition, disorder, or disease is eradicated. Rather, treatment also contemplates cases where a treatment regimen containing one of the compounds, compositions, or pharmaceutical formulations of the present disclosure provides an improved clinical outcome. The improved clinical outcome may include one or more of the following: abatement, lessening, and/or alleviation of one or more symptoms that result from or are associated with the condition, disorder, or disease to be treated; decreased occurrence of one or more symptoms; improved quality of life; diminishment of the extent of the condition, disorder, or disease; reaching or establishing a stabilized state (i.e., not worsening) of the condition, disorder, or disease; delay or slowing of the progression of the condition, disorder, or disease; amelioration or palliation of the state of the condition, disorder, or disease; partial or total remission; and improvement in survival (whether increase in the overall survival rate or prolonging of survival when compared to expected survival if the subject were not receiving the treatment). For example, the disclosure encompasses treatment that reduces one or more symptoms of and/or cognitive deficit associated with or caused by a brain injury.

The terms “deuterated” and “deuteration” refer to replacement of one or more non-ionizable hydrogen atoms in a chemical compound/moiety with deuterium. A deuterated chemical compound/group/moiety may be fully deuterated (i.e., all the non-ionizable hydrogen atoms in the chemical compound/moiety are replaced with deuterium) or partially deuterated (i.e., one or more non-ionizable hydrogen atoms, but not all the non-ionizable hydrogen atoms, in the chemical compound/group/moiety are replaced with deuterium).

The terms “halogenated” and “halogenation” refer to replacement of one or more non-ionizable hydrogen atoms in a chemical compound/moiety with halogen such as fluorine. A halogenated chemical compound/group/moiety may be fully halogenated (i.e., all the non-ionizable hydrogen atoms in the chemical compound/moiety are replaced with halogen such as fluorine) or partially deuterated (i.e., one or more non-ionizable hydrogen atoms, but not all the non-ionizable hydrogen atoms, in the chemical compound/group/moiety are replaced with halogen such as fluorine).

The terms “derivative” and “derivatives” refer to chemical compounds/groups/moieties with a structure similar to that of a parent compound/group/moiety but different from it in respect to one or more components, functional groups, atoms, etc. Optionally, the derivatives retain certain functional attributes of the parent compound/group/moiety. Optionally, the derivatives can be formed from the parent compound/group/moiety by chemical reaction(s). The differences between the derivatives and the parent compound/group/moiety can include, but are not limited to, replacement of one or more functional groups with one or more different functional groups or introducing or removing one or more substituents of hydrogen atoms.

The term “alkyl” refers to univalent groups derived from alkanes (i.e., acyclic saturated hydrocarbons) by removal of a hydrogen atom from any carbon atom. Alkyl groups can be linear or branched. Suitable alkyl groups can have one to 30 carbon atoms, i.e., C₁-C₃₀ alkyl. If the alkyl is branched, it is understood that at least three carbon atoms are present.

The term “alkenyl” refers to univalent groups derived from alkenes by removal of a hydrogen atom from any carbon atom. Alkenes are unsaturated hydrocarbons that contain at least one carbon-carbon double bond. Alkenyl groups can be linear or branched. Suitable alkenyl groups can have two to 30 carbon atoms, i.e., C₂-C₃₀ alkenyl. If the alkenyl is branched, it is understood that at least three carbon atoms are present.

The term “alkynyl” refers to univalent groups derived from alkynes by removal of a hydrogen atom from any carbon atom. Alkynes are unsaturated hydrocarbons that contain at least one carbon-carbon triple bond. Alkynyl groups can be linear or branched. Suitable alkynyl groups can have two to 30 carbon atoms, i.e., C₂-C₃₀ alkynyl. If the alkynyl is branched, it is understood that at least four carbon atoms are present.

The term “heteroalkyl” refers to alkyl groups where one or more carbon atoms are replaced with a heteroatom such as, O, N, S, or Si. Optionally, the nitrogen and/or sulphur heteroatom(s) may be oxidized, and the nitrogen heteroatom(s) may be quaternized. Heteroalkyl groups can be linear or branched. Suitable heteroalkyl groups can have one to 30 carbon atoms, i.e., C₁-C₃₀ heteroalkyl. If the heteroalkyl is branched, it is understood that at least one carbon atom and at least one heteroatom are present.

The term “aryl” refers to univalent groups derived from arenes by removal of a hydrogen atom from a ring atom. Arenes are monocyclic or polycyclic aromatic hydrocarbons. In polycyclic arenes, the rings can be attached together in a pendant manner, a fused manner, or a combination thereof. Accordingly, in polycyclic aryl groups, the rings can be attached together in a pendant manner, a fused manner, or a combination thereof. Suitable aryl groups can have six to 30 carbon atoms, i.e., C₆-C₃₀ aryl. The number of “members” of an aryl group refers to the total number of carbon atoms in the ring(s) of the aryl group.

The term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2). Heteroarenes can be monocyclic or polycyclic. In polycyclic heteroarenes, the rings can be attached together in a pendant manner, a fused manner, or a combination thereof. Accordingly, in polycyclic heteroaryl groups, the rings can be attached together in a pendant manner, a fused manner, or a combination thereof. Suitable heteroaryl groups can have one to 30 carbon atoms, i.e., C₁-C₃₀ heteroaryl. The number of “members” of a heteroaryl group refers to the total number of carbon atom(s) and heteroatom(s) in the ring(s) of the heteroaryl group.

“Carbocycle” or “carbocyclyl” refers to mono- and polycyclic ring systems containing only carbon atoms as ring atoms. The mono- and polycyclic ring systems may be aromatic, non-aromatic (saturated or unsaturated), or a mixture of aromatic and non-aromatic rings. Carbocyclyls are univalent, derived from carbocycles by removal of a hydrogen atom from a ring atom. Carbocycles include arenes; carbocyclyls include aryls. In polycyclic carbocycles or carbocyclyls, the rings can be attached together in a pendant manner (i.e., two rings are connected by a single bond), a spiro manner (i.e., two rings are connected through a defining single common atom), a fused manner (i.e., two rings share two adjacent atoms; in other words, two rings share one covalent bond), a bridged manner (i.e., two rings share three or more atoms, separating the two bridgehead atoms by a bridge containing at least one atom), or a combination thereof. Suitable carbocycle or carbocyclyl groups can have three to 30 carbon atoms, i.e., C₃-C₃₀ carbocycle or carbocyclyl. The number of “members” of a carbocycle or carbocyclyl group refers to the total number of carbon atoms in the ring(s) of the carbocycle or carbocyclyl group.

“Heterocycle” or “heterocyclyl” refers to mono- and polycyclic ring systems containing at least one carbon atom and one or more heteroatoms independently selected from elements like nitrogen, oxygen, and sulfur, as ring atoms. Optionally, the nitrogen and/or sulphur heteroatom(s) may be oxidized, and the nitrogen heteroatom(s) may be quaternized. The mono- and polycyclic ring systems may be aromatic, non-aromatic, or a mixture of aromatic and non-aromatic rings. Heterocyclyls are univalent, derived from heterocycles by removal of a hydrogen atom from a ring atom. Heterocycles include heteroarenes; heterocyclyls include heteroaryls. In polycyclic heterocycle or heterocyclyl groups, the rings can be attached together in a pendant manner (i.e., two rings are connected by a single bond), a spiro manner (i.e., two rings are connected through a defining single common atom), a fused manner (i.e., two rings share two adjacent atoms; in other words, two rings share one covalent bond), a bridged manner (i.e., two rings share three or more atoms, separating the two bridgehead atoms by a bridge containing at least one atom), or a combination thereof. Suitable heterocycle or heterocyclyl groups can have one to 30 carbon atoms, i.e., C₁-C₃₀ heterocycle or heterocyclyl. The number of “members” of a heterocycle or heterocyclyl group refers to the total number of carbon atom(s) and heteroatom(s) in the ring(s) of the heterocycle or heterocyclyl group.

As used herein, the terms “halogen” and “halo” refer to fluorine, chlorine, bromine, and iodine.

As used herein, “haloalkyl” refers to halogen-substituted alkyl groups. Optionally, the haloalkyl groups contain one halogen substituent. Optionally, the haloalkyl groups contain multiple halogen substituents, i.e., polyhaloalkyl. In some examples, the haloalkyl groups contain one or more fluorine substituents.

As used herein, “haloalkenyl” refers to halogen-substituted alkenyl groups. Optionally, the haloalkenyl groups contain one halogen substituent. Optionally, the haloalkenyl groups contain multiple halogen substituents. In some examples, the haloalkenyl groups contain one or more fluorine substituents.

As used herein, “haloalkynyl” refers to halogen-substituted alkynyl groups. Optionally, the haloalkynyl groups contain one halogen substituent. Optionally, the haloalkynyl groups contain multiple halogen substituents. In some examples, the haloalkynyl groups contain one or more fluorine substituents.

As used herein, “halocarbocyclyl” refers to halogen-substituted carbocyclyl groups. Optionally, the halocarbocyclyl groups contain one halogen substituent. Optionally, the halocarbocyclyl groups contain multiple halogen substituents. In some examples, the halocarbocyclyl groups contain one or more fluorine substituents.

As used herein, “haloheterocyclyl” refers to halogen-substituted heterocyclyl groups. Optionally, the haloheterocyclyl groups contain one halogen substituent. Optionally, the haloheterocyclyl groups contain multiple halogen substituents. In some examples, the haloheterocyclyl groups contain one or more fluorine substituents.

As used herein, “haloaryl” refers to halogen-substituted aryl groups. Optionally, the haloaryl groups contain one halogen substituent. Optionally, the haloaryl groups contain multiple halogen substituents. In some examples, the haloaryl groups contain one or more fluorine substituents.

As used herein, “haloheteroaryl” refers to halogen-substituted heteroaryl groups. Optionally, the haloheteroaryl groups contain one halogen substituent. Optionally, the haloheteroaryl groups contain multiple halogen substituents. In some examples, the haloheteroaryl groups contain one or more fluorine substituents.

The term “substituted,” as used herein, means that the chemical group or moiety contains one or more substituents replacing the hydrogen atom(s) in the original chemical group or moiety. It is understood that any substitution is in accordance with a permitted valence of the substituted atom and the substituent and that the substitution results in a stable compound, e.g., a compound that does not spontaneously undergo transformation such as by rearrangement, cyclization, elimination, etc., under room temperature. Unless otherwise specified, the substituents are R groups. The R groups, on each occurrence, can be independently selected from halogen, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COOH, —SO₃H, —CH₂SO₂CH₃, —PO₃H₂, —OPO₃H₂, —P(═O)(OR^{G1})(OR^{G2}), —OP(═O)(OR^{G1})(OR^{G2}), —BR^{G1}(OR^{G2}), —B(OR^{G1})(OR^{G2}), —Si(R^{G1})(R^{G2})(R^{G3}), —C(R^{G1})(R^{G2})(R^{G3}), —N[(R^{G1})(R^{G2})(R^{G3})], and -GR^{G1}, in which -G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G3}—, —C(═S)—, —C(═S)S—, —SC(═S)—, —SC(═S)S—, —C(═NR^{G2})—, —C(═NR^{G2})O—, —C(═NR^{G2} NR^{G3}—, —OC(═NR^{G2})—, —NR^{G2}C(═NR^{G3})—, —NR^{G2}SO₂—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G3}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O—, —SO₂NR^{G2}—, —BR^{G2}—, or —PR^{G2}—, wherein each occurrence of R^{G1}, R^{G2}, and R^{G3} is independently selected from hydrogen, halogen, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, and haloheteroaryl. Optionally, two R groups on the same atom can join together with that atom to form a cyclic moiety, such as a carbocycle or a heterocycle. Alternatively, two R groups on the same atom can merge into one oxygen (═O) or sulfur (═S) atom.

The term “optionally substituted,” as used herein, means that substitution is optional, and therefore it is possible for the designated atom/chemical group/compound to be unsubstituted.

As used herein, “ester” refers to —C(═O)OR^{c1} or —OC(═O)R^{c2}, wherein R^{c1} and R^{c2} are independently selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle.

As used herein, “amino” refers to —NR^{d1}R^{d2}, wherein R^{d1} and R^{d2} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. When R^{d1} and R^{d2} are each hydrogen, the amino group is a primary amino group.

As used herein, “acyl” refers —C(═O)R^{e}, wherein R^{e} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle.

As used herein, “amide” refers to —C(═O)NR^{f1}R^{f2}, wherein R^{f1} and R^{f2} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. When R^{f1} and R^{f2} are each hydrogen, the amide group is a carbamoyl group.

As used herein, “carbonate ester” refers to —OC(═O)ORⁱ, wherein Rⁱ is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle.

As used herein, “carbamate” refers to —OC(═O)NR^{j1}R^{j2} or —NR^{k}[(C═O)OR^{l}], wherein R^{j1}, R^{j2}, R^{k}, and R^{l} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle.

As used herein, “sulfinyl” refers to —S(═O)R^{m}, wherein R^{m} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle.

As used herein, “sulfonyl” refers to —S(═O)₂Rⁿ, wherein Rⁿ is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle.

As used herein, “thioester” refers to —C(═O)SR^{o1} or —SC(═O)R^{o2}, wherein R^{o1} and R^{o2} are independently selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle.

As used herein, “sulfonamide” refers to —S(═O)₂NR^{p1}R^{p2}, wherein R^{p1} and R^{p2} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. When R^{p1} and R^{p2} are each hydrogen, the amide group is a sulfamoyl group.

As used herein, “thiol” refers to the univalent radical —SH.

As used herein, “sulfonate” refers to —SO₃⁻.

As used herein, “silyl” refers to the univalent radical derived from silane by removal of a hydrogen atom, i.e., —SiH₃.

As used herein, “carbonate” refers to —O(C═O)OH.

As used herein, the term “stereoisomer” refers to compounds made up of the same atoms having the same bond order but having different three-dimensional arrangements of atoms which are not interchangeable. As used herein, the term “enantiomer” refers to a pair of stereoisomers that are non-superimposable mirror images of one another. As used herein, the term “diastereomer” refers to two stereoisomers that are not mirror images but also not superimposable. The terms “racemate” and “racemic mixture” refer to a mixture of enantiomers. The term “chiral center” refers to a carbon atom to which four different groups are attached. Choice of the appropriate chiral column, eluent, and conditions necessary for effective separation of stereoisomers, such as a pair of enantiomers, is well known to one of ordinary skill in the art (e.g., Jacques et al., Enantiomers, Racemates, and Resolutions, John Wiley and Sons, Inc., 1981).

As used herein, the term “pharmaceutically acceptable” refers to compounds, materials, compositions, or formulations which are, within the scope of sound medical judgment, suitable for use in contact with the tissues of human beings and non-human animals without excessive toxicity, irritation, allergic response, or other problems or complications that commensurate with a reasonable benefit/risk ratio, in accordance with the guidelines of regulatory agencies of a certain country, such as the Food and Drug Administration (FDA) in the United States or its corresponding agencies in countries other than the United States (e.g., the European Medicines Agency (EMA) in Europe, the National Medical Products Administration (NMPA) in China).

As used herein, the term “salt” refers to acid or base salts of the original compound. In some cases, the salt is formed in situ during preparation of the original compound, i.e., the designated synthetic chemistry procedures produce the salt instead of the original compound. In some cases, the salt is obtained via modification of the original compound. In some cases, the salt is obtained via ion exchange with an existing salt of the original compound. Examples of salts include, but are not limited to, mineral or organic acid salts of basic residues such as amines, as well as alkali or organic salts of acidic residues such as carboxylic acids and phosphonic acids. For original compounds containing a basic residue, the salts can be prepared by treating the compounds with an appropriate amount of a non-toxic inorganic or organic acid; alternatively, the salts can be formed in situ during preparation of the original compounds. Exemplary salts of the basic residue include salts with an inorganic acid selected from hydrochloric, hydrobromic, sulfuric, sulfamic, phosphoric, and nitric acids or with an organic acid selected from acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic acids. For original compounds containing an acidic residue, the salts can be prepared by treating the compounds with an appropriate amount of a non-toxic base; alternatively, the salts can be formed in situ during preparation of the original compounds. Exemplary salts of the acidic residue include salts with a base selected from ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, and histidine. Optionally, the salts can be prepared by reacting the free acid or base form of the original compounds with a stoichiometric amount or more of an appropriate base or acid, respectively, in water or an aqueous solution, an organic solvent or an organic solution, or a mixture thereof. Lists of exemplary pharmaceutically acceptable salts can be found in Remington's Pharmaceutical Sciences, 20^{th} Ed., Lippincott Williams & Wilkins, Baltimore, MD, 2000 as well as Handbook of Pharmaceutical Salts: Properties, Selection, and Use, Stahl and Wermuth, Eds., Wiley-VCH, Weinheim, 2002.

As used herein, the term “excipient” refers to any components present in the pharmaceutical formulations disclosed herein, other than the active ingredient (i.e., a compound or composition of the present disclosure).

As used herein, the term “effective amount” of a material refers to a nontoxic but sufficient amount of the material to provide the desired result. The exact amount required may vary from subject to subject, depending on the species, age, and general condition of the subject, the severity of the condition, disorder, or disease that is being treated, the active ingredient or therapy used, and the like.

As used herein, the term “physiological pH” refers to the pH that normally prevails in the human body in the absence of pathological states. Typically, it ranges between 7.35 and 7.45.

### II. Compounds

The present disclosure describes neurosteroids and prodrugs of the neurosteroids. Also disclosed are deuterated or halogenated analogs of (1) the neurosteroids and (2) the prodrugs of the neurosteroids.

In general, the neurosteroids have a 20-carbon skeleton, as shown in Formula I. Notably, the neurosteroids contain an oxime group at the C20 position. This structure feature is different from many endogenous neurosteorids, which contain a ketone group at the C20 position. Installation of the oxime group eliminates ketone reduction, which is one of the metabolic pathways of endogenous neurosteorids, such as allopregnanolone, while retaining or even enhancing the biological activities, including modulation of the GABA receptors.

In general, the prodrugs have a higher solubility in an aqueous medium than their corresponding neurosteroid parent compounds. In some embodiments, the prodrugs are capable of self-immolative cleavage in response to environmental pH changes, releasing the corresponding neurosteroid parent compounds. In some embodiments, the prodrugs are stable in an acidic aqueous medium but exhibit a wide range of release kinetics in human plasma. In some embodiments, the prodrugs contain a phosphate group and are readily cleavable by phosphatases.

To the extent that chemical formulas described herein contain one or more unspecified chiral centers, the formulas are intended to encompass all stable stereoisomers, enantiomers, and diastereomers. Such compounds can exist as a single enantiomer, a racemic mixture, a mixture of diastereomers, or combinations thereof. It is also understood that the chemical formulas encompass all tautomeric forms if tautomerization occurs.

Methods of making exemplary compounds are disclosed in subsequent sections and exemplified by the Examples. The synthetic methods disclosed herein are compatible with a wide variety of functional groups and starting materials. Thus, a wide variety of compounds can be obtained from the disclosed methods.

Optionally, the alkyl groups described herein have 1-30 carbon atoms, i.e., C₁-C₃₀ alkyl. In some forms, the C₁-C₃₀ alkyl can be a linear C₁-C₃₀ alkyl or a branched C₃-C₃₀ alkyl. Optionally, the alkyl groups have 1-20 carbon atoms, i.e., C₁-C₂₀ alkyl. In some forms, the C₁-C₂₀ alkyl can be a linear C₁-C₂₀ alkyl or a branched C₃-C₂₀ alkyl. Optionally, the alkyl groups have 1-10 carbon atoms, i.e., C₁-C₁₀ alkyl. In some forms, the C₁-C₁₀ alkyl can be a linear C₁-C₁₀ alkyl or a branched C₃-C₁₀ alkyl. Optionally, the alkyl groups have 1-6 carbon atoms, i.e., C₁-C₆ alkyl. In some forms, the C₁-C₆ alkyl can be a linear C₁-C₆ alkyl or a branched C₃-C₆ alkyl. Representative straight chain alkyl groups include methyl, ethyl, n-propyl, n-butyl, n-pentyl, n-hexyl, n-heptyl, n-octyl, n-nonyl, and the like. Representative branched alkyl groups include isopropyl, sec-butyl, isobutyl, tert-butyl, isopentyl, and the like.

Optionally, the alkenyl groups described herein have 2-30 carbon atoms, i.e., C₂-C₃₀ alkenyl. In some forms, the C₂-C₃₀ alkenyl can be a linear C₂-C₃₀ alkenyl or a branched C₃-C₃₀ alkenyl. Optionally, the alkenyl groups have 2-20 carbon atoms, i.e., C₂-C₂₀ alkenyl. In some forms, the C₂-C₂₀ alkenyl can be a linear C₂-C₂₀ alkenyl or a branched C₃-C₂₀ alkenyl. Optionally, the alkenyl groups have 2-10 carbon atoms, i.e., C₂-C₁₀ alkenyl. In some forms, the C₂-C₁₀ alkenyl can be a linear C₂-C₁₀ alkenyl or a branched C₃-C₁₀ alkenyl. Optionally, the alkenyl groups have 2-6 carbon atoms, i.e., C₂-C₆ alkenyl. In some forms, the C₂-C₆ alkenyl can be a linear C₂-C₆ alkenyl or a branched C₃-C₆ alkenyl. Representative alkenyl groups include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like.

Optionally, the alkynyl groups described herein have 2-30 carbon atoms, i.e., C₂-C₃₀ alkynyl. In some forms, the C₂-C₃₀ alkynyl can be a linear C₂-C₃₀ alkynyl or a branched C₄-C₃₀ alkynyl. Optionally, the alkynyl groups have 2-20 carbon atoms, i.e., C₂-C₂₀ alkynyl. In some forms, the C₂-C₂₀ alkynyl can be a linear C₂-C₂₀ alkynyl or a branched C₄-C₂₀ alkynyl. Optionally, the alkynyl groups have 2-10 carbon atoms, i.e., C₂-C₁₀ alkynyl. In some forms, the C₂-C₁₀ alkynyl can be a linear C₂-C₁₀ alkynyl or a branched C₄-C₁₀ alkynyl. Optionally, the alkynyl groups have 2-6 carbon atoms, i.e., C₂-C₆ alkynyl. In some forms, the C₂-C₆ alkynyl can be a linear C₂-C₆ alkynyl or a branched C₄-C₆ alkynyl. Representative alkynyl groups include ethynyl, propynyl, 1-butynyl, 2-butynyl, 1-pentynyl, 2-pentynyl, 3-methyl-1-butynyl, and the like.

Optionally, the heteroalkyl groups described herein have 1-30 carbon atoms, i.e., C₁-C₃₀ heteroalkyl. In some forms, the C₁-C₃₀ heteroalkyl can be a linear C₁-C₃₀ heteroalkyl or a branched C₁-C₃₀ heteroalkyl. Optionally, the heteroalkyl groups have 1-20 carbon atoms, i.e., C1-C₂₀ heteroalkyl. In some forms, the C₁-C₂₀ heteroalkyl can be a linear C₁-C₂₀ heteroalkyl or a branched C1-C₂₀ heteroalkyl. Optionally, the heteroalkyl groups have 1-10 carbon atoms, i.e., C₁-C₁₀ heteroalkyl. In some forms, the C₁-C₁₀ heteroalkyl can be a linear C₁-C₁₀ heteroalkyl or a branched C₁-C₁₀ heteroalkyl. Optionally, the heteroalkyl groups have 1-6 carbon atoms, i.e., C₁-C₆ heteroalkyl. In some forms, the C1-C₆ heteroalkyl can be a linear C₁-C₆ heteroalkyl or a branched C₁-C₆ heteroalkyl.

Optionally, the aryl groups described herein have 6-30 carbon atoms, i.e., C₆-C₃₀ aryl. Optionally, the aryl groups have 6-20 carbon atoms, i.e., CO—C₂₀ aryl. Optionally, the aryl groups have 6-12 carbon atoms, i.e., C₆-C₁₂ aryl. Representative aryl groups include phenyl, naphthyl, and biphenyl.

Optionally, the heteroaryl groups described herein have 1-30 carbon atoms, i.e., C₁-C₃₀ heteroaryl. Optionally, the heteroaryl groups have 1-20 carbon atoms, i.e., C₁-C₂₀ heteroaryl. Optionally, the heteroaryl groups have 1-11 carbon atoms, i.e., C₁-C₁₁ heteroaryl. Optionally, the heteroaryl groups have 1-5 carbon atoms, i.e., C₁-C₅ heteroaryl. Optionally, the heteroaryl groups are 5-20 membered heteroaryl groups. Optionally, the heteroaryl groups are 5-12 membered heteroaryl groups. Optionally, the heteroaryl groups are 5 or 6 membered heteroaryl groups. Representative heteroaryl groups include furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

Optionally, the carbocyclyl groups described herein have 3-30 carbon atoms, i.e., C₃-C₃₀ carbocyclyl. Optionally, the carbocyclyl groups described herein have 3-20 carbon atoms, i.e., C₃-C₂₀ carbocyclyl. Optionally, the carbocyclyl groups described herein have 3-12 carbon atoms, i.e., C₃-C₁₂ carbocyclyl. Representative saturated carbocyclyl groups include cyclopropyl, cyclobutyl, cyclopentyl, cyclohexyl, and the like. Representative unsaturated carbocyclyl groups include cyclopentenyl, cyclohexenyl, and the like.

Optionally, the heterocyclyl groups described herein have 1-30 carbon atoms, i.e., C₁-C₃₀ heterocyclyl. Optionally, the heterocyclyl groups described herein have 1-20 carbon atoms, i.e., C₁-C₂₀ heterocyclyl. Optionally, the heterocyclyl groups described herein have 1-11 carbon atoms, i.e., C₁-C₁₁ heterocyclyl. Optionally, the heterocyclyl groups described herein have 1-6 carbon atoms, i.e., C₁-C₆ heterocyclyl. Optionally, the heterocyclyl groups are 3-20 membered heterocyclyl groups. Optionally, the heterocyclyl groups are 3-12 membered heterocyclyl groups. Optionally, the heteroaryl groups are 4-7 membered heterocyclyl groups.

The optionally substituted groups described in the chemical formulas described herein (e.g., Formulas I-III and their sub-formulas), on each occurrence when not specified, may have one or more substituents in the form of the R groups described above.

The R groups, on each occurrence, can be independently selected from halogen, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COOH, —SO₃H, —CH₂SO₂CH₃, —PO₃H₂, —OPO₃H₂, —P(═O)(OR^{G1})(OR^{G2}), —OP(═O)(OR^{G1})(OR^{G2}), —BR^{G1}(OR^{G2}), —B(OR^{G1})(OR^{G2}), —Si(R^{G1})(R^{G2})(R^{G3}), —C(R^{G1})(R^{G2})(R^{G3}), —N[(R^{G1})(R^{G2})(R^{G3})], and -GR^{G1}, in which -G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G3}—, C(═S)—, —C(═S)S—, —SC(═S)—, —SC(═S)S—, —C(═NR^{G2})—, —C(═NR^{G2})O—OC(═NR^{G2})NR^{G3}—, —OC(═NR^{G2})—, —NR^{G2}C(═NR^{G3})—, —NR^{G2}SO₂—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G3}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O—, —SO₂NR^{G2}—, —BR^{G2}—, or —PR^{G2}—, wherein each occurrence of R^{G1}, R^{G2}, and R^{G3} is independently selected from hydrogen, halogen, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, and haloheteroaryl. Optionally, two R groups on the same atom can join together with that atom to form a cyclic moiety, such as a carbocycle or a heterocycle. Alternatively, two R groups on the same atom can merge into one oxygen (═O) or sulfur (═S) atom.

In some examples, the R groups are independently selected from halogen, nitro, cyano, hydroxyl, formyl, carboxyl, thiol, ═O (counting as two R groups), ═S (counting as two R groups), sulfamoyl, alkyl (such as methyl, ethyl, isopropyl, tert-butyl), haloalkyl (such as trifluoromethyl), alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, arylalkyl (such as benzyl), alkylaryl, alkyloxy (such as methoxy, ethoxy), haloalkyloxy (such as trifluoromethoxy), aryloxy, alkylcarbonyl (such as acetyl), arylcarbonyl (such as benzoyl), alkylcarbonyloxy (such as acetoxy), arylcarbonyloxy (such as benzoyloxy), alkyloxycarbonyl (such as methoxycarbonyl, ethoxycarbonyl, isopropoxycarbonyl), aryloxycarbonyl, primary amino, alkylamino (such as methylamino, ethylamino, dimethylamino, diethylamino, N-methyl-N-ethylamino), alkylammonium (such as trimethylammonium), alkylcarbonylamino (such as acetylamino), arylcarbonylamino (such as benzoylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), and N-alkylsulfamoyl (such as N-methylsulfamoyl, N-ethylsulfamoyl, N,N-dimethylsulfamoyl, N,N-diethylsulfamoyl, N-methyl-N-ethylsulfamoyl).

In some examples, the R groups are independently selected from halogen, nitro, cyano, hydroxyl, trifluoromethyl, methoxy, ethoxy, trifluoromethoxy, primary amino, formyl, carboxyl, carbamoyl, thiol, ═O, ═S, sulfamoyl, acetyl, acetoxy, methoxycarbonyl, ethoxycarbonyl, isopropoxycarbonyl, methylamino, ethylamino, dimethylamino, diethylamino, N-methyl-N-ethylamino, trimethylammonium, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, N-methylsulfamoyl, N-ethylsulfamoyl, NN-dimethylsulfamoyl, N,N-diethylsulfamoyl, N-methyl-N-ethylsulfamoyl, benzyl, benzoyl, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, and haloheteroaryl.

In some examples, the R groups are independently selected from halogen, ═O, ═S, alkyl, haloalkyl, carbocyclyl, halocarbocyclyl, aryl, haloaryl, heterocyclyl, and haloheterocyclyl.

As used herein, “alkyloxy” refers to a hydroxyl group substituted by an alkyl group at the oxygen atom. Exemplary alkyloxy groups include, but are not limited to, methoxy, ethoxy, n-propoxy, i-propoxy, n-butoxy, s-butoxy, t-butoxy, n-pentoxy, and s-pentoxy. As used herein, “haloalkyloxy” refers to a hydroxyl group substituted by a haloalkyl group at the oxygen atom. An example of haloalkyloxy is trifluoromethoxy. As used herein, “aryloxy” refers to a hydroxyl group substituted by an aryl group at the oxygen atom.

As used herein, “alkylcarbonyl” refers to an alkyl group attached through a carbonyl bridge (—C(═O)—). As used herein, “arylcarbonyl” refers to an aryl group attached through a carbonyl bridge.

As used herein, “alkylcarbonyloxy” refers to a hydroxyl group substituted by an alkylcarbonyl group at the oxygen atom of the hydroxyl group. As used herein, “arylcarbonyloxy” refers to a hydroxyl group substituted by an arylcarbonyl group at the oxygen atom of the hydroxyl group.

As used herein, “alkyloxycarbonyl” refers to an alkyloxy group attached through a carbonyl bridge. As used herein, “aryloxycarbonyl” refers to an aryloxy group attached through a carbonyl bridge.

As used herein, “alkylamino” refers to a primary amino group substituted by one or two alkyl groups. When the primary amino group is substituted by two alkyl groups, the two alkyl groups can be the same or different. An example of alkylamino is methylamino (i.e., —NH—CH₃).

As used herein, “alkylammonium” refers to a primary ammonium group substituted by one, two, or three alkyl groups. When the primary ammonium group is substituted by two or three alkyl groups, the two or three alkyl groups can be the same or different. An example of alkylammonium is trimethylammonium (i.e., —N(CH₃)₃⁺).

As used herein, “alkylcarbonylamino” refers to a primary amino group substituted by one alkylcarbonyl group. As used herein, “arylcarbonylamino” refers to a primary amino group substituted by one arylcarbonyl group.

As used herein, “N-alkylcarbamoyl” refers to a carbamoyl group (—C(═O)—NH₂) substituted by one or two alkyl groups at the nitrogen atom. When the carbamoyl group is substituted by two alkyl groups, the two alkyl groups can be the same or different.

As used herein, “alkylthio” refers to a thiol group substituted by an alkyl group at the sulfur atom. An example of alkylthio is methylthio (i.e., —S—CH₃).

As used herein, “alkylsulfinyl” refers to an alkyl group attached through a sulfinyl bridge (—S(═O)—). As used herein, “alkylsulfonyl” refers to an alkyl group attached through a sulfonyl bridge (—S(═O)₂—).

As used herein, “N-alkylsulfamoyl” refers to a sulfamoyl group (—S(═O)₂—NH₂) substituted by one or two alkyl groups at the nitrogen atom. When the sulfamoyl group is substituted by two alkyl groups, the two alkyl groups can be the same or different.

### A. Neurosteroids

The neurosteroids have a structure of Formula I or a pharmaceutically acceptable salt, hydrate, or hydrated salt of Formula I,

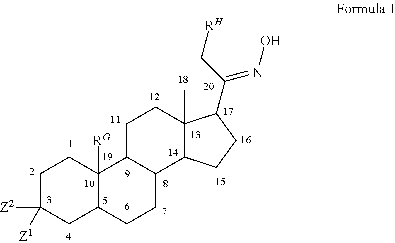

wherein R^{G} is hydrogen or methyl;
wherein R^{H} is hydrogen or an optionally substituted nitrogen-containing 5-membered heteroaryl;
wherein Z¹ is —OR^{X}, wherein R^{X} is selected from hydrogen, acyl, ester, thioester, and amide; and
wherein Z² is methyl, halogen-substituted methyl, methoxymethyl, or halogen-substituted methoxymethyl.

The numberings of carbon atoms in Formula I apply to all sub-formulas of Formula I, including Formulas I-1 and I-2.

Notably, the neurosteroids contain an oxime group at the C20 position. This structure feature is different from many endogenous neurosteorids, which contain a ketone group at the C20 position. Installation of the oxime group eliminates ketone reduction, which is one of the metabolic pathways of endogenous neurosteorids, such as allopregnanolone, while retaining the biological activities, including modulation of the GABA receptors. For example, as the present inventors have shown, installation of the oxime group may selectively potentiate a GABA_{A} receptor containing the delta (δ) subunit, compared to a GABA_{A} receptor without the delta (δ) subunit. Notably, the delta (δ) subunit of the GABA_{A} receptor plays a critical role in neurological disorders such as depression, especially postpartum depression, and anxiety. Accordingly, the neurosteroids disclosed herein have enhanced metabolic stability compared to their corresponding ketone versions. Additionally, they may have enhanced selectivity towards GABA_{A} receptors containing the delta (δ) subunit.

In some embodiments, Formula I is in the form of Formula I-1. In some embodiments, Formula I is in the form of Formula I-2.

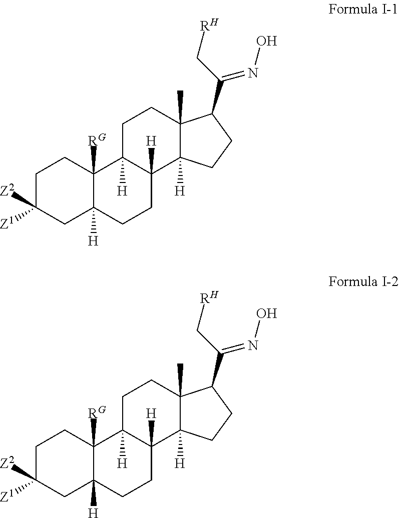

In some embodiments, R^{G} is hydrogen. In some embodiments, R^{G} is methyl.

In some embodiments, R^{H} is hydrogen. In some embodiments, R^{H} is optionally substituted nitrogen-containing 5-membered heteroaryl.

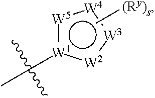

In some embodiments, R^{H} is wherein W¹, W², W³, W⁴, and W⁵ are independently and individually C, CH, N, or NH, wherein at least one of W¹, W², W³, W⁴, and W⁵ is C or CH, wherein at least one of W¹, W², W³, W⁴, and W⁵ is N or NH, wherein s is 0, 1, 2, 3, or 4, wherein R^{Y}, at each occurrence, is independently and individually halogen, nitro, cyano, hydroxyl, formyl, carboxyl, sulfamoyl, alkyl such as methyl, haloalkyl such as CF₃, alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, arylalkyl, alkylaryl, alkyloxy, haloalkyloxy, aryloxy, haloaryloxy, alkylcarbonyl, arylcarbonyl, alkylcarbonyloxy, arylcarbonyloxy, alkyloxycarbonyl, aryloxycarbonyl, primary amino, alkylamino, alkylammonium, alkylcarbonylamino, arylcarbonylamino, carbamoyl, N-alkylcarbamoyl, alkylthio, alkylsulfinyl, alkylsulfonyl, or N-alkylsulfamoyl. It is understood by those skill in the art that when an R group is present, it replaces the hydrogen atom at the ring atom that the R^{Y} group connects to. In some embodiments, W¹ is N. In some embodiments, W¹ is C. In some embodiments, s is 0. In some embodiments, s is 1. In some embodiments, R^{Y}, at each occurrence, is independently and individually halogen, cyano, alkyl such as methyl, or haloalkyl such as CF₃. In some embodiments, R^{Y}, at each occurrence, is independently and individually halogen, cyano, methyl, or CF₃. In some embodiments, W¹ is N, and s is 1. In some embodiments, W¹ is N, s is 1, and R^{y} is selected from halogen, methyl, CF₃, and cyano. In some embodiments, W¹ is N, and s is 0.

In some embodiments, at least two of W¹, W², W³, W⁴, and W⁵ are independently N or NH. For example, R^{H} is a diazole optionally substituted by R^{y}. In one implementation, R^{H} is an unsubstituted diazole. In one implementation, R^{H} is a diazole mono-substituted by halogen, methyl, CF₃, or cyano. In some embodiments, at least three of W¹, W², W³, W⁴, and W⁵ are independently N or NH. For example, R^{H} is a triazole optionally substituted by R^{y}. In one implementation, R^{H} is an unsubstituted triazole. In one implementation, R^{H} is a triazole mono-substituted by halogen, methyl, CF₃, or cyano. In some embodiments, at least four of W¹, W², W³, W⁴, and W⁵ are independently N or NH. For example, R^{H} is a tetrazole optionally substituted by R^{y}. In one implementation, R^{H} is an unsubstituted tetrazole. In one implementation, R^{H} is a tetrazole mono-substituted by halogen, methyl, CF₃, or cyano.

In some embodiments, R^{H} is selected from

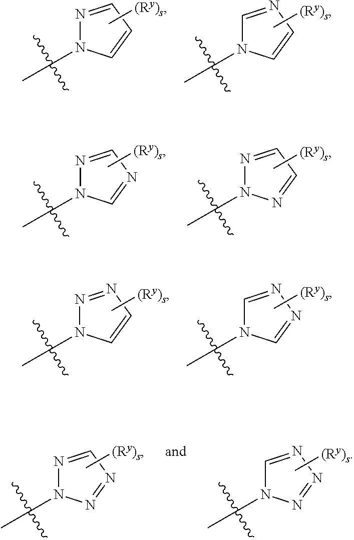

In some embodiments, R^{H} is

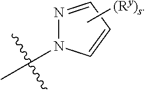

In some embodiments, R^{H} is

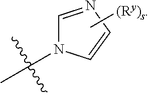

In some embodiments, R^{H} is

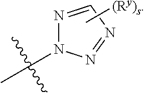

In some embodiments, R^{y}, at each occurrence, is independently and individually halogen, cyano, alkyl such as methyl, or haloalkyl such as CF₃. In some embodiments, R^{y}, at each occurrence, is independently and individually halogen, cyano, methyl, and CF₃.

In some embodiments, R^{H} is selected from

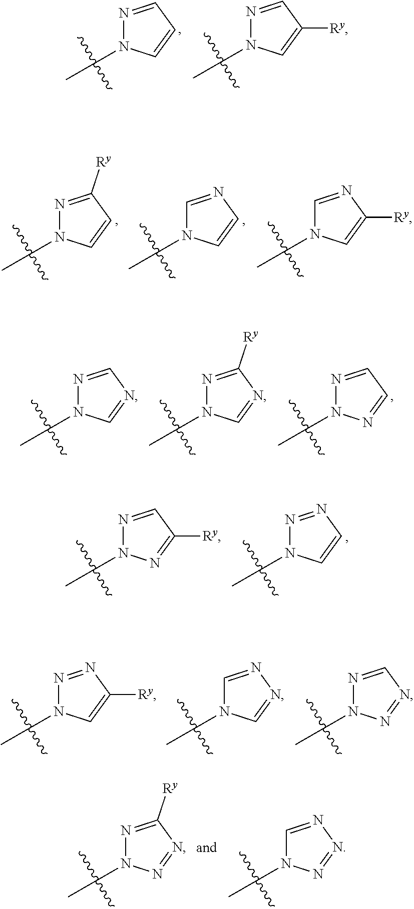

In some embodiments, R^{H} is

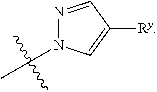

In some embodiments, R^{H} is

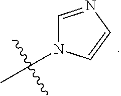 [SMILES: *n1ccnc1]

In some embodiments, R^{H} is

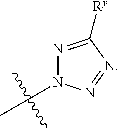

In some embodiments, R^{y}, if present, is halogen, cyano, alkyl such as methyl, or haloalkyl such as CF₃. In some embodiments, R^{y}, if present, is halogen, cyano, methyl, or CF₃.

In some embodiments, R^{H} is

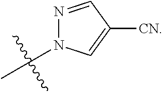 [SMILES: *n1cc(C#N)cn1]

In some embodiments, R^{H} is

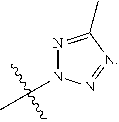 [SMILES: *n1nnc(C)n1]

In some embodiments, R^{H} is

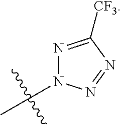 [SMILES: *n1nnc(C(F)(F)F)n1]

In some embodiments, R^{X} is hydrogen, i.e., Z¹ is —OH.

In some embodiments, R^{X} is acyl. For example, R^{X} is —C(═O)R^{e}, wherein R^{e} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle. In some embodiments, R^{e} is C₁-C₆ alkyl, halogen-substituted C₁-C₆ alkyl, C₃-C₆ cycloalkyl, or halogen-substituted C₃-C₆ cycloalkyl. In some embodiments, R^{e} is selected from methyl, —CF₃, ethyl, isopropyl, hexafluoroisopropyl, 1-butyl, and cyclopropyl. In some embodiments, R^{e} is —CF₃. In some embodiments, R^{e} is isopropyl.

In some embodiments, R^{X} is ester. For example, R^{X} is —C(═O)OR^{c1}, wherein R^{c1} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. In some embodiments, R^{c1} is C₁-C₆ alkyl, halogen-substituted C₁-C₆ alkyl, C₃-C₆ cycloalkyl, or halogen-substituted C₃-C₆ cycloalkyl. In some embodiments, R^{c1} is selected from methyl, —CF₃, ethyl, isopropyl, hexafluoroisopropyl, t-butyl, and cyclopropyl. In some embodiments, R^{c1} is —CF₃. In some embodiments, R^{c1} is isopropyl.

In some embodiments, R^{X} is thioester. For example, R^{X} is —C(═O)SR^{o1}, wherein R^{o1} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. In some embodiments, R^{o1} is C₁-C₆ alkyl, halogen-substituted C₁-C₆ alkyl, C₃-C₆ cycloalkyl, or halogen-substituted C₃-C₆ cycloalkyl. In some embodiments, R^{o1} is selected from methyl, —CF₃, ethyl, isopropyl, hexafluoroisopropyl, t-butyl, and cyclopropyl. In some embodiments, R^{o1} is —CF₃. In some embodiments, R^{o1} is isopropyl.

In some embodiments, R^{X} is amide. For example, R^{X} is —C(═O)NR^{f1}R^{f2}, wherein R^{f1} and R^{f2} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. In some embodiments, R^{f1} and R^{f2} are hydrogen. In some embodiments, R^{f1} and R^{f2} are methyl. In some embodiments, R^{f1}is hydrogen and R^{f2} is methyl.

In some embodiments, Z² is methyl. In some embodiments, Z² is halogen-substituted methyl, such as —CH₂F, —CHF₂, or —CF₃.

In some embodiments, Z² is methoxymethyl. In some embodiments, Z² is halogen-substituted methoxymethyl, such as —CH₂OCF₃.

In some embodiments, Z² is selected from methyl, —CH₂F, —CHF₂, —CF₃, and —CH₂OCH₃. In some embodiments, Z² is selected from methyl, —CF₃, and —CH₂OCH₃.

Exemplary neurosteroids include, but are not limited to, the following:

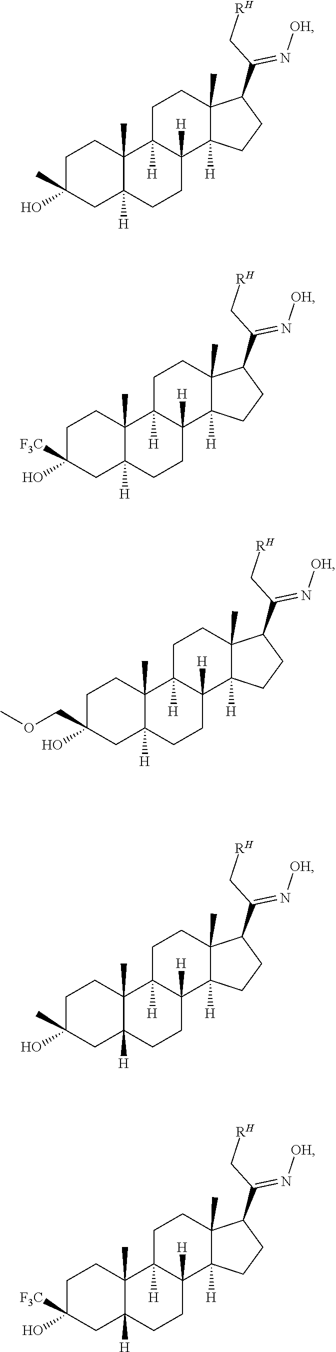

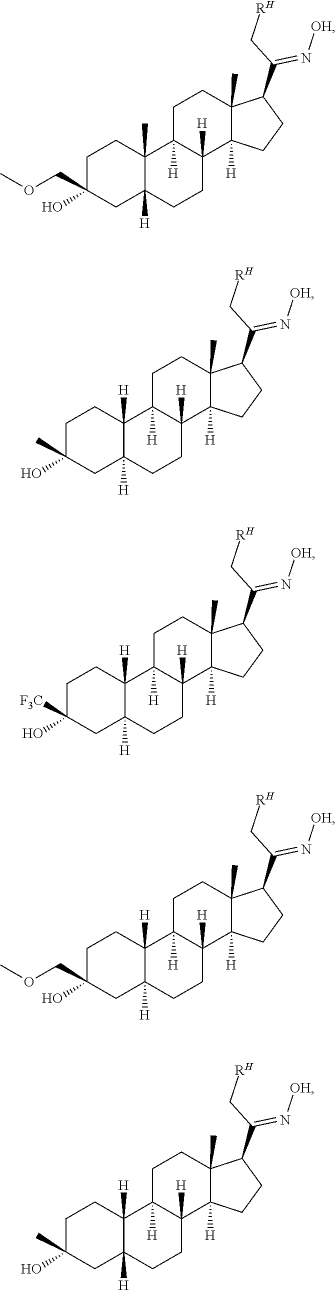

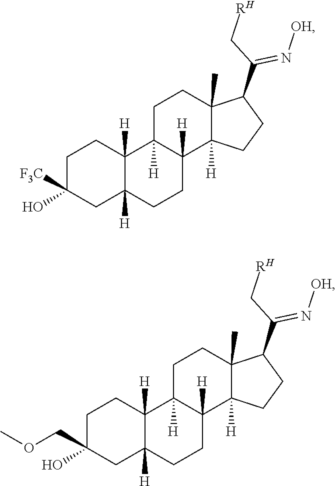

and pharmaceutically acceptable salts, hydrates, or hydrated salts thereof. In some embodiments, R^{H} is H. In some embodiments, R^{H} is

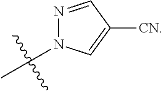 [SMILES: *n1cc(C#N)cn1]

In some embodiments, R^{H} is

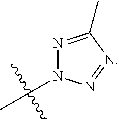 [SMILES: *n1nnc(C)n1]

In some embodiments, R^{H} is

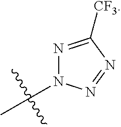 [SMILES: *n1nnc(C(F)(F)F)n1]

In some embodiments, R^{H} is

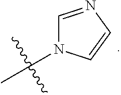 [SMILES: *n1ccnc1]

In some embodiments, the neurosteroid is selected from

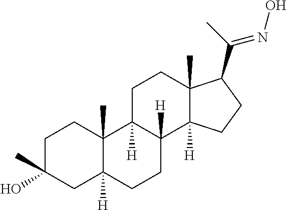

or a pharmaceutically acceptable salt, hydrate, or hydrated salt thereof. In some embodiments, the neurosteroid is

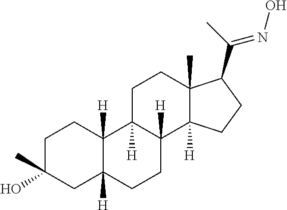

or a pharmaceutically acceptable salt, hydrate, or hydrated salt thereof. In some embodiments, the neurosteroid is

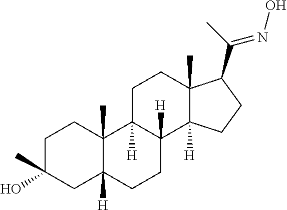

or a pharmaceutically acceptable salt, hydrate, or hydrated salt thereof. In some embodiments, the neurosteroid is

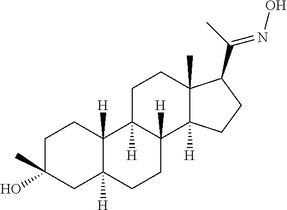

or a pharmaceutically acceptable salt, hydrate, or hydrated salt thereof. In some embodiments, the neurosteroid is

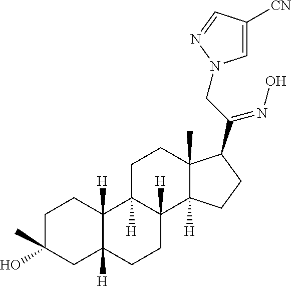

or a pharmaceutically acceptable salt, hydrate, or hydrated salt thereof. In some embodiments, the neurosteroid is

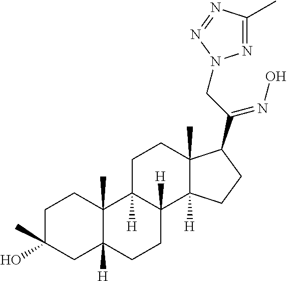

or a pharmaceutically acceptable salt, hydrate, or hydrated salt thereof. In some embodiments, the neurosteroid is

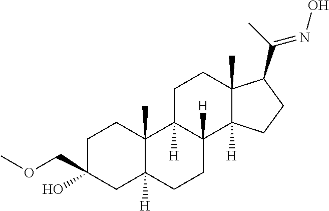

or a pharmaceutically acceptable salt, hydrate, or hydrated salt thereof. In some embodiments, the neurosteroid is

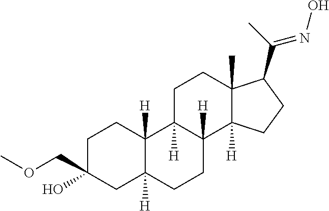

or a pharmaceutically acceptable salt, hydrate, or hydrated salt thereof. In some embodiments, the neurosteroid is

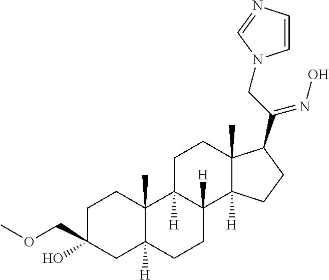

or a pharmaceutically acceptable salt, hydrate, or hydrated salt thereof. In some embodiments, the neurosteroid is

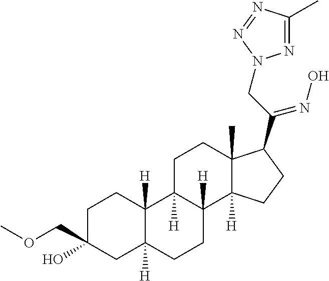

or a pharmaceutically acceptable salt, hydrate, or hydrated salt thereof. In some embodiments, the neurosteroid is

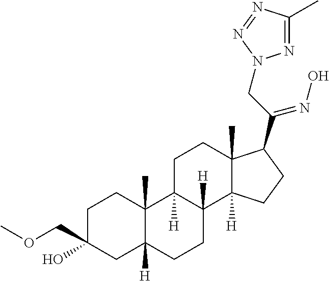

or a pharmaceutically acceptable salt, hydrate, or hydrated salt thereof.

### B. Non-Phosphate Prodrugs of the Neurosteroids

In some embodiments, the prodrugs of the neurosteroids have a structure of Formula II or a pharmaceutically acceptable salt, hydrate, or hydrated salt of Formula II,

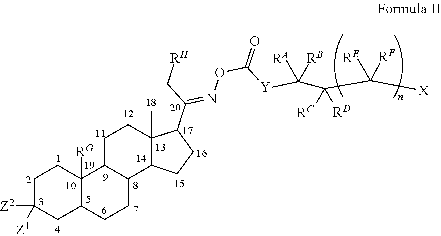

wherein R^{G}, R^{H}, Z¹, and Z² are the same as described above in Formula I; and
wherein:
(3) X is OH or NR¹R²,
   Y is O or NR³,
   R^{A}, R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
   R¹, R², and R³ are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen;
(2) X is NR¹R²,
   Y is O or NR³,
   R¹ joins R^{C} or R^{E} to form a 4-7 membered, optionally substituted heterocycle,
   R² is hydrogen,
   R^{A}, R^{B}, R^{C}, R^{D}, R^{E}, and R^{F}, on each occurrence when not joined by R¹ to form the heterocycle, are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
   R³ is independently selected from hydrogen, optionally substituted alkyl, optionally substituted heteroalkyl, optionally substituted carbocyclyl, optionally substituted heterocyclyl, optionally substituted aryl, and optionally substituted heteroaryl; or
(3) X is OH or NR¹R²,
   Y is NR³,
   R³ joins R^{A} to form a 4-7 membered, optionally substituted heterocycle,
   R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
   R¹ and R² are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

The numberings of carbon atoms in Formula II apply to all sub-formulas of Formula II.

In some embodiments, the prodrugs are in a non-salt form as shown in Formula II. In some embodiments, the prodrugs are in a salt form. In some embodiments, the prodrugs are in an HCl salt form.

In general, the prodrugs described in this section have a higher solubility in an aqueous medium than their corresponding neurosteroid parent compounds. In some embodiments, the prodrugs are capable of self-immolative cleavage in response to environmental Ph changes, releasing the corresponding neurosteroid parent compounds. In some embodiments, the prodrugs are stable in an acidic aqueous medium but exhibit a wide range of release kinetics in human plasma.

In some embodiments, Formula II is in the form of Formula II-1. In some embodiments, the prodrugs are in a non-salt form as shown in Formula II-1. In some embodiments, the prodrugs are in a salt form. In some embodiments, the prodrugs are in an HCl salt form.

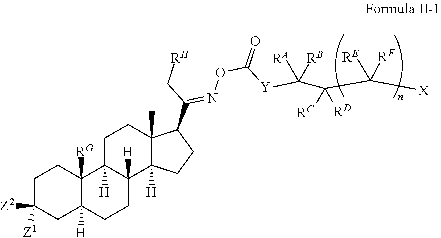

In some embodiments, Formula II is in the form of Formula II-2. In some embodiments, the prodrugs are in a non-salt form as shown in Formula II-2. In some embodiments, the prodrugs are in a salt form. In some embodiments, the prodrugs are in an HCl salt form.

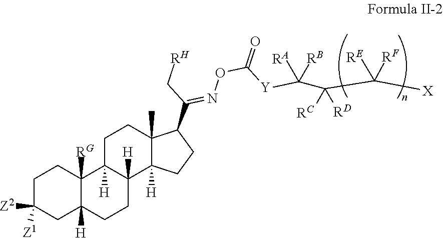

In some embodiments, n is 0, and Formula II is in the form of Formula IIA. In some embodiments, the prodrugs are in a non-salt form as shown in Formula II-A. In some embodiments, the prodrugs are in a salt form. In some embodiments, the prodrugs are in an HCl salt form.

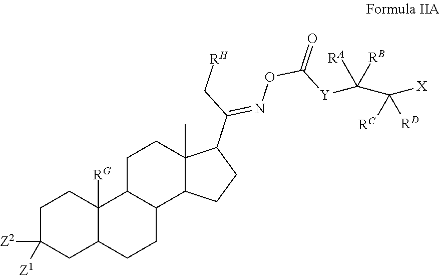

In some embodiments, Formula IIA is in the form of Formula IIA-1. In some embodiments, the prodrugs are in a non-salt form as shown in Formula IIA-1. In some embodiments, the prodrugs are in a salt form. In some embodiments, the prodrugs are in an HCl salt form.

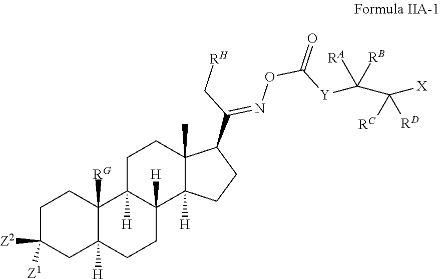

In some embodiments, Formula IIA is in the form of Formula IIA-2. In some embodiments, the prodrugs are in a non-salt form as shown in Formula IIA-2. In some embodiments, the prodrugs are in a salt form. In some embodiments, the prodrugs are in an HCl salt form.

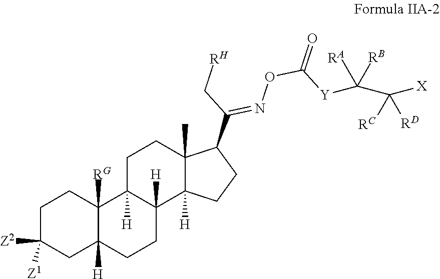

In some embodiments, n is 1, and Formula II is in the form of Formula IIB. In some embodiments, the prodrugs are in a non-salt form as shown in Formula IIB. In some embodiments, the prodrugs are in a salt form. In some embodiments, the prodrugs are in an HCl salt form.

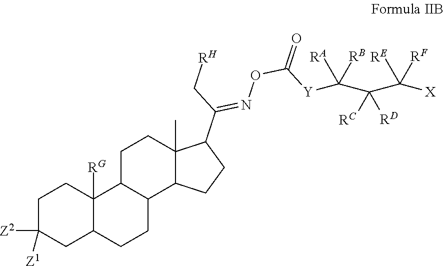

In some embodiments, Formula IIB is in the form of Formula IIB-1. In some embodiments, the prodrugs are in a non-salt form as shown in Formula IIB-1. In some embodiments, the prodrugs are in a salt form. In some embodiments, the prodrugs are in an HCl salt form.

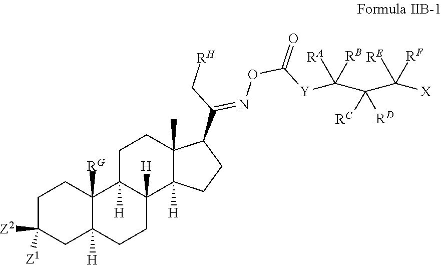

In some embodiments, Formula IIB is in the form of Formula IIB-2. In some embodiments, the prodrugs are in a non-salt form as shown in Formula IIB-2. In some embodiments, the prodrugs are in a salt form. In some embodiments, the prodrugs are in an HCl salt form.

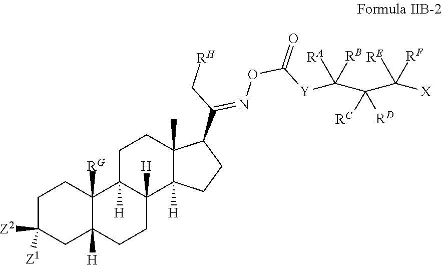

### 1. The Neurosteroid Moiety

The neurosteroid moiety

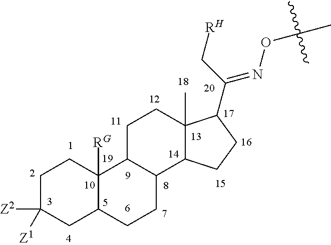

is the same as described above in Section II(A) of the Detailed Description.

In some embodiments, the neurosteroid moiety is in the following form.

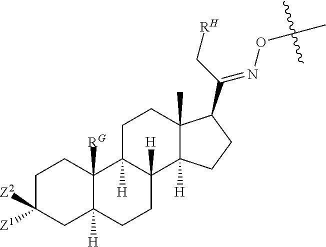

In some embodiments, the neurosteroid moiety is in the following form.

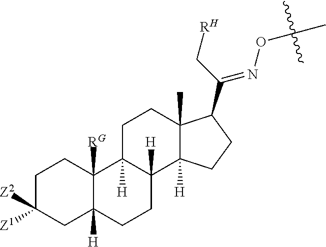

In some embodiments, R^{G} is hydrogen. In some embodiments, R^{G} is methyl.

In some embodiments, R^{H} is hydrogen. In some embodiments, R^{H} is optionally substituted nitrogen-containing 5-membered heteroaryl.

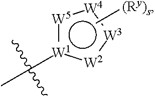

In some embodiments, R^{H} is wherein W¹, W², W³, W⁴, and W¹ are independently and individually C, CH, N, or NH, wherein at least one of W¹, W², W³, W⁴, and W⁵ is C or CH, wherein at least one of W¹, W², W³, W⁴, and W⁵ is N or NH, wherein s is 0, 1, 2, 3, or 4, wherein R^{y}, at each occurrence, is independently and individually halogen, nitro, cyano, hydroxyl, formyl, carboxyl, sulfamoyl, alkyl such as methyl, haloalkyl such as CF₃, alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, arylalkyl, alkylaryl, alkyloxy, haloalkyloxy, aryloxy, haloaryloxy, alkylcarbonyl, arylcarbonyl, alkylcarbonyloxy, arylcarbonyloxy, alkyloxycarbonyl, aryloxycarbonyl, primary amino, alkylamino, alkylammonium, alkylcarbonylamino, arylcarbonylamino, carbamoyl, N-alkylcarbamoyl, alkylthio, alkylsulfinyl, alkylsulfonyl, or N-alkylsulfamoyl. It is understood by those skill in the art that when an R group is present, it replaces the hydrogen atom at the ring atom that the R^{y} group connects to. In some embodiments, W¹ is N. In some embodiments, W¹ is C. In some embodiments, s is 0. In some embodiments, s is 1. In some embodiments, R^{y}, at each occurrence, is independently and individually halogen, cyano, alkyl such as methyl, or haloalkyl such as CF₃. In some embodiments, R^{y}, at each occurrence, is independently and individually halogen, cyano, methyl, or CF₃. In some embodiments, W¹is N, and s is 1. In some embodiments, W¹ is N, s is 1, and R^{y} is selected from halogen, methyl, CF₃, and cyano. In some embodiments, W¹ is N, and s is 0.

In some embodiments, at least two of W¹, W², W³, W⁴, and W⁵ are independently N or NH. For example, R^{H} is a diazole optionally substituted by R^{y}. In one implementation, R^{H} is an unsubstituted diazole. In one implementation, R^{H} is a diazole mono-substituted by halogen, methyl, CF₃, or cyano. In some embodiments, at least three of W¹, W², W³, W⁴, and W⁵ are independently N or NH. For example, R^{H} is a triazole optionally substituted by R^{Y}. In one implementation, R^{H} is an unsubstituted triazole. In one implementation, R^{H} is a triazole mono-substituted by halogen, methyl, CF₃, or cyano. In some embodiments, at least four of W¹, W², W³, W⁴, and W⁵ are independently N or NH. For example, R^{H} is a tetrazole optionally substituted by R^{Y}. In one implementation, R^{H} is an unsubstituted tetrazole. In one implementation, R^{H} is a tetrazole mono-substituted by halogen, methyl, CF₃, or cyano.

In some embodiments, R^{H} is selected from

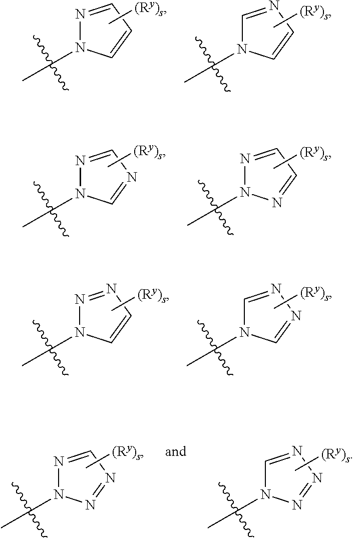

In some embodiments, R^{H} is

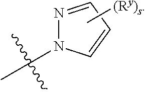

In some embodiments, R^{H} is

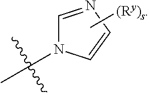

In some embodiments, R^{H} is

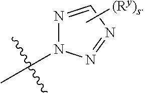

In some embodiments, R^{y}, at each occurrence, is independently and individually halogen, cyano, alkyl such as methyl, or haloalkyl such as CF₃. In some embodiments, R^{y}, at each occurrence, is independently and individually halogen, cyano, methyl, and CF₃.

In some embodiments, R^{H} is selected from

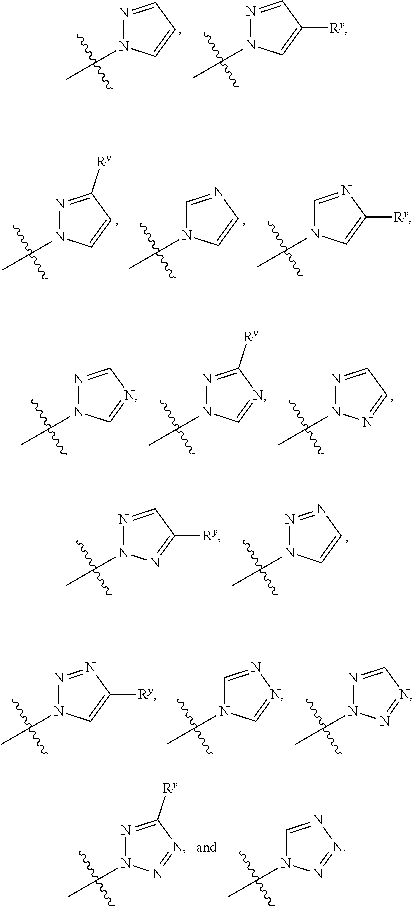

In some embodiments R^{H} is

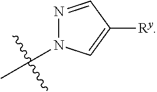

In some embodiments, R^{H} is

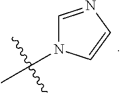 [SMILES: *n1ccnc1]

In some embodiments, R^{H} is

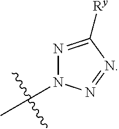

In some embodiments, R^{y}, if present, is halogen, cyano, alkyl such as methyl, or haloalkyl such as CF₃. In some embodiments, R^{y}, if present, is halogen, cyano, methyl, or CF₃.

In some embodiments, R^{H} is

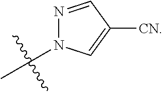 [SMILES: *n1cc(C#N)cn1]

In some embodiments, R^{X} is

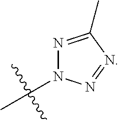 [SMILES: *n1nnc(C)n1]

In some embodiments, R^{X} is

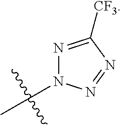 [SMILES: *n1nnc(C(F)(F)F)n1]

In some embodiments, R^{X} is hydrogen, i.e., Z¹ is —OH.

In some embodiments, R^{X} is acyl. For example, R^{X} is —C(═O)R^{e}, wherein R^{e} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle. In some embodiments, R^{e} is C₁-C₆ alkyl, halogen-substituted C₁-C₆ alkyl, C₃-C₆ cycloalkyl, or halogen-substituted C₃-C₆ cycloalkyl. In some embodiments, R^{e} is selected from methyl, —CF₃, ethyl, isopropyl, hexafluoroisopropyl, 1-butyl, and cyclopropyl. In some embodiments, R^{e} is —CF₃. In some embodiments, R^{e} is isopropyl.

In some embodiments, R^{X} is ester. For example, R^{X} is —C(═O)OR^{c1}, wherein R^{c1} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. In some embodiments, R^{c1} is C₁-C₆ alkyl, halogen-substituted C₁-C₆ alkyl, C₃-C₆ cycloalkyl, or halogen-substituted C₃-C₆ cycloalkyl. In some embodiments, R^{c1} is selected from methyl, —CF₃, ethyl, isopropyl, hexafluoroisopropyl, t-butyl, and cyclopropyl. In some embodiments, R^{c1} is —CF₃. In some embodiments, R^{c1} is isopropyl.

In some embodiments, R^{X} is thioester. For example, R^{X} is —C(═O)SR^{o1}, wherein R^{o1} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. In some embodiments, R^{o1} is C₁-C₆ alkyl, halogen-substituted C₁-C₆ alkyl, C₃-C₆ cycloalkyl, or halogen-substituted C₃-C₆ cycloalkyl. In some embodiments, R^{o1} is selected from methyl, —CF₃, ethyl, isopropyl, hexafluoroisopropyl, t-butyl, and cyclopropyl. In some embodiments, R^{o1} is —CF₃. In some embodiments, R^{o1} is isopropyl.

In some embodiments, R^{X} is amide. For example, R^{X} is —C(═O)NR^{f1}R^{f2}, wherein R^{f1} and R^{f2} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. In some embodiments, R^{f1} and R^{f2} are hydrogen. In some embodiments, R^{f1} and R^{f2} are methyl. In some embodiments, R^{f1}is hydrogen and R^{f2} is methyl.

In some embodiments, Z² is methyl. In some embodiments, Z² is halogen-substituted methyl, such as —CH₂F, —CHF₂, or —CF₃.

In some embodiments, Z² is methoxymethyl. In some embodiments, Z² is halogen-substituted methoxymethyl, such as —CH₂OCF₃.

In some embodiments, Z² is selected from methyl, —CH₂F, —CHF₂, —CF₃, and —CH₂OCH₃. For example, Z² is selected from methyl, —CF₃, and —CH₂OCH₃.

In some embodiments, the neurosteroid moiety is selected from the following:

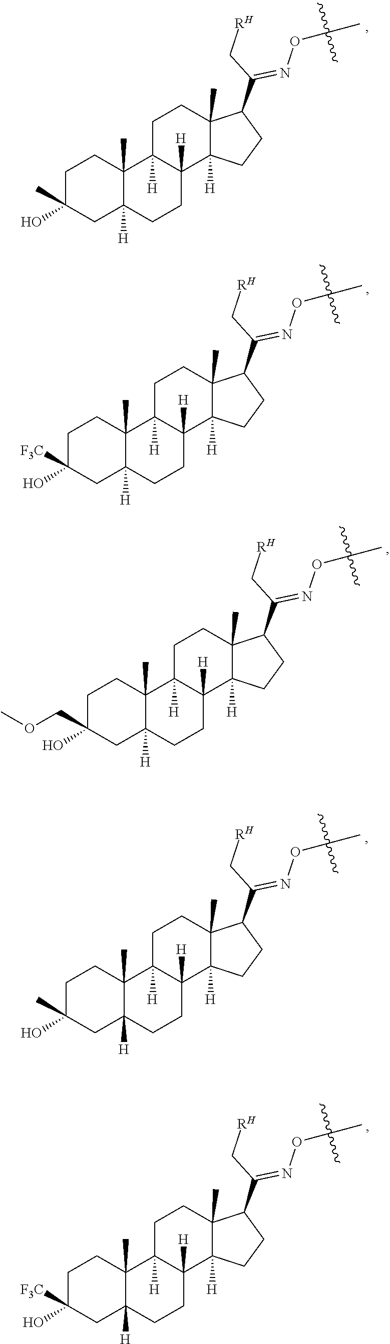

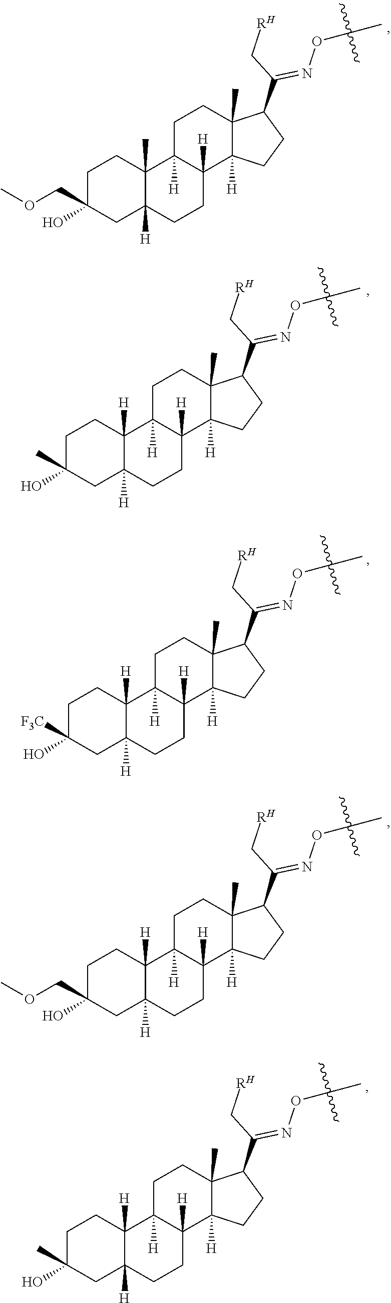

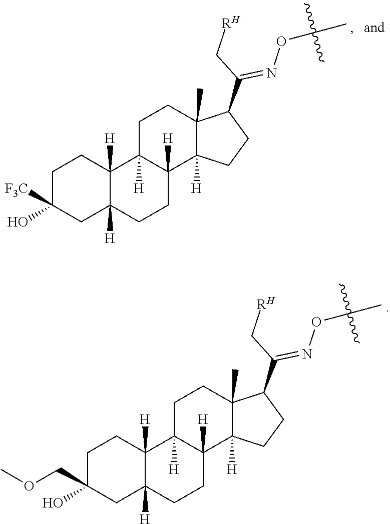

In some embodiments, R^{H} is H. In some embodiments, R^{H} is

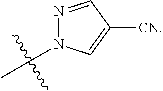 [SMILES: *n1cc(C#N)cn1]

In some embodiments, R^{H} is

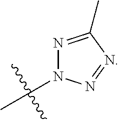 [SMILES: *n1nnc(C)n1]

In some embodiments, R^{H} is

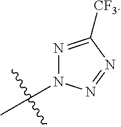 [SMILES: *n1nnc(C(F)(F)F)n1]

In some embodiments, R^{H} is

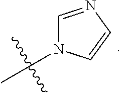 [SMILES: *n1ccnc1]

In some embodiments, the neurosteroid moiety is

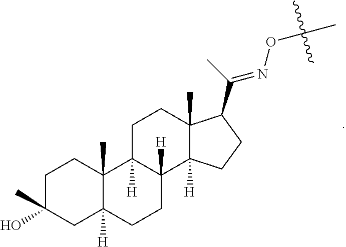

In some embodiments, the neurosteroid moiety is

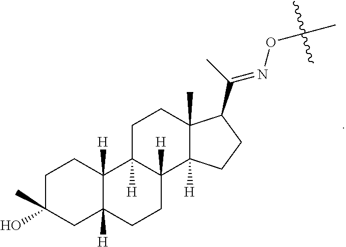

In some embodiments, the neurosteroid moiety is

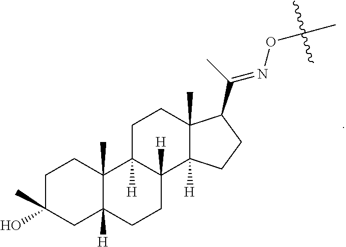

In some embodiments, the neurosteroid moiety is

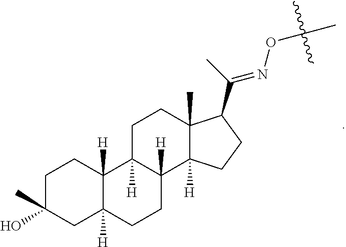

In some embodiments, the neurosteroid moiety is

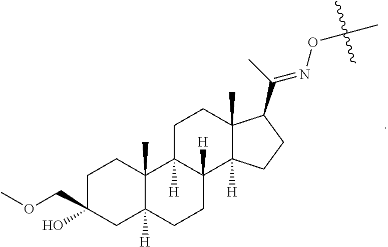

In some embodiments, the neurosteroid moiety is

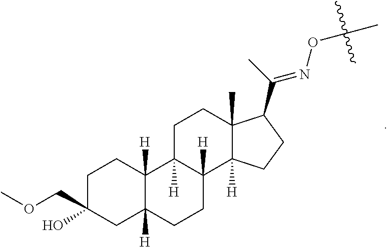

In some embodiments, the neurosteroid moiety is

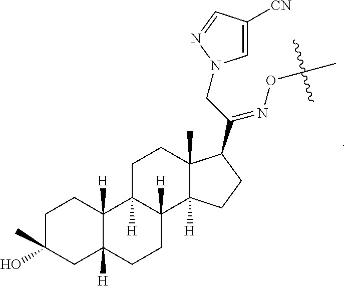

In some embodiments, the neurosteroid moiety is

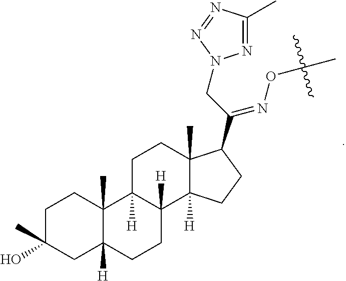

In some embodiments, the neurosteroid moiety is

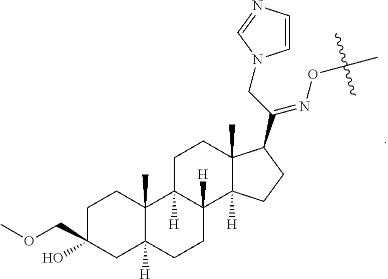

In some embodiments, the neurosteroid moiety is

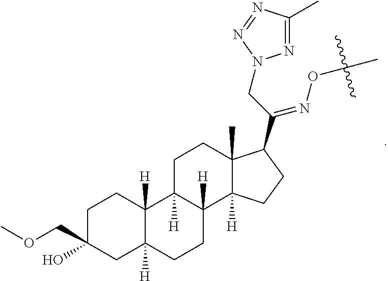

In some embodiments, the neurosteroid moiety is

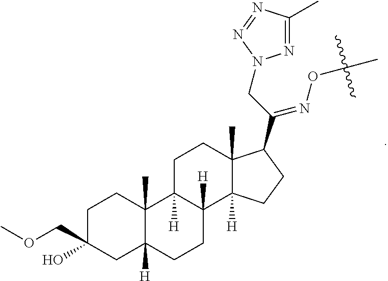

### 2. The Prodrug Moiety at the C20-Oxime

### Group I

In some embodiments, the prodrugs have the following features:
X is OH or NR¹R²,
Y is O or NR³,
R^{A}, R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹, R², and R³ are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

In some embodiments, X is OH. In some embodiments, X is NR¹R². In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃⁺, and R² is hydrogen.

In some embodiments, Y is O. In some embodiments, Y is NR³. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

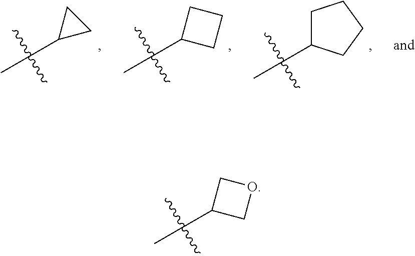

In some embodiments, R³ is

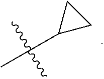 [SMILES: *C1CC1]

In some embodiments, R³ is

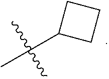 [SMILES: *C1CCC1]

In some embodiments, R³ is

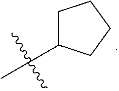 [SMILES: *C1CCCC1]

In some embodiments, R³ is

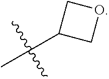 [SMILES: *C1COC1]

In some embodiments, n is 0, i.e., both R^{E} and R^{F} are absent. In some embodiments, n is 1, i.e., both R^{E} and R^{F} are present.

Optionally, at least one of R^{A} and R^{B} is hydrogen. In some embodiments, both R^{A} and R^{B} are hydrogen. In some embodiments, R^{A} is optionally substituted alkyl, and R^{B} is hydrogen. In some embodiments, R^{A} is optionally substituted C₁-C₄ alkyl, and R^{B} is hydrogen. In some embodiments, R^{A} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{B} is hydrogen. In some embodiments, R^{A} is methyl, and R^{B} is hydrogen.

Optionally, at least one of R^{A} and R^{B} is optionally substituted alkyl. In some embodiments, at least one of R^{A} and R^{B} is optionally substituted C₁-C₄ alkyl. In some embodiments, at least one of R^{A} and R^{B} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, C(CH₃)₃, CH₂CH₂NH₂, CH₂CH₂NHCH₃, CH₂CH₂N(CH₃)₂, and CH₂CH₂N(CH₃)₃⁺. In some embodiments, at least one of R^{A} and R^{B} is methyl. In some embodiments, each of R^{A} and R^{B} is, independently, an optionally substituted alkyl. In some embodiments, each of R^{A} and R^{B} is, independently, an optionally substituted C₁-C₄ alkyl. In some embodiments, each of R^{A} and R^{B} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{A} and R^{B} is methyl.

Optionally, at least one of R^{C} and R^{D} is hydrogen. In some embodiments, both R^{C} and R^{D} are hydrogen. In some embodiments, R^{C} is optionally substituted alkyl, and R^{D} is hydrogen. In some embodiments, R^{C} is optionally substituted C₁-C₄ alkyl, and R^{D} is hydrogen. In some embodiments, R^{C} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{D} is hydrogen. In some embodiments, R^{C} is methyl, and R^{D} is hydrogen.

Optionally, at least one of R^{C} and R^{D} is optionally substituted alkyl. In some embodiments, at least one of R^{C} and R^{D} is optionally substituted C₁-C₄ alkyl. In some embodiments, at least one of R^{C} and R^{D} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{C} and R^{D} is methyl. In some embodiments, each of R^{C} and R^{D} is, independently, an optionally substituted alkyl. In some embodiments, each of R^{C} and R^{D} is, independently, an optionally substituted C₁-C₄ alkyl. In some embodiments, each of R^{C} and R^{D} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{C} and R^{D} is methyl.

In some embodiments, R^{A}, R^{B}, R^{C}, and R^{D} are hydrogen.

Optionally, at least one of R^{E} and R^{F} is hydrogen. In some embodiments, both R^{E} and R^{F} are hydrogen. In some embodiments, R^{E} is optionally substituted alkyl, and R^{F} is hydrogen. In some embodiments, R^{E} is optionally substituted C₁-C₄ alkyl, and R^{F} is hydrogen. In some embodiments, R^{E} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{F} is hydrogen. In some embodiments, R^{E} is methyl, and R^{F} is hydrogen.

Optionally, at least one of R^{E} and R^{F} is optionally substituted alkyl. In some embodiments, at least one of R^{E} and R^{F} is optionally substituted C₁-C₄ alkyl. In some embodiments, at least one of R^{E} and R^{F} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{E} and R^{F} is methyl. In some embodiments, each of R^{E} and R^{F} is, independently, an optionally substituted alkyl. In some embodiments, each of R^{E} and R^{F} is, independently, an optionally substituted C₁-C₄ alkyl. In some embodiments, each of R^{E} and R^{F} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{E} and R^{F} is methyl.

In some embodiments, n is 0, X is NR¹R², and Y is NR³. In some embodiments, n is 0, X is NR¹R², Y is NR³, and R^{A} and R^{B} are hydrogen. In some embodiments, n is 0, X is NR¹R², Y is NR³, R^{A} is methyl, and R^{B} is hydrogen. In some embodiments, n is 0, X is NR¹R², Y is NR³, and R^{A} and R^{B} are methyl. In some embodiments, n is 0, X is NR¹R², Y is NR³, and R^{C} and R^{D} are hydrogen. In some embodiments, n is 0, X is NR¹R², Y is NR³, R^{C} is methyl, and R^{D} is hydrogen. In some embodiments, n is 0, X is NR¹R², Y is NR³, and R^{C} and R^{D} are methyl. In some embodiments, n is 0, X is NR¹R², Y is NR³, and R^{A}, R^{B}, R^{C}, and R^{D} are hydrogen. In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃⁺, and R² is hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

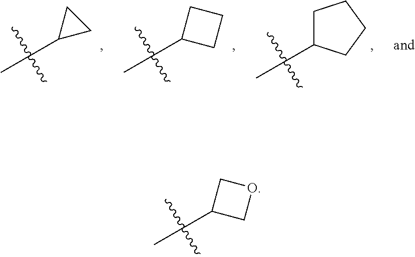

In some embodiments R³ is

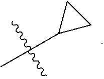 [SMILES: *C1CC1]

In some embodiments, R³ is

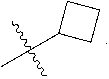 [SMILES: *C1CCC1]

In some embodiments, R³ is

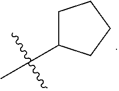 [SMILES: *C1CCCC1]

In some embodiments, R³ is

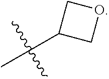 [SMILES: *C1COC1]

In some embodiments, n is 1, X is NR¹R², and Y is NR³. In some embodiments, n is 1, X is NR¹R², Y is NR³, and R^{A} and R^{B} are hydrogen. In some embodiments, n is 1, X is NR¹R², Y is NR³, R^{A} is methyl, and R^{B} is hydrogen. In some embodiments, n is 1, X is NR¹R², Y is NR³, and R^{A} and R^{B} are methyl. In some embodiments, n is 1, X is NR¹R², Y is NR³, and R^{C} and R^{D} are hydrogen. In some embodiments, n is 1, X is NR¹R², Y is NR³, R^{C} is methyl, and R^{D} is hydrogen. In some embodiments, n is 1, X is NR¹R², Y is NR³, and R^{C} and R^{D} are methyl. In some embodiments, n is 1, X is NR¹R², Y is NR³, and R^{E} and R^{F} are hydrogen. In some embodiments, n is 1, X is NR¹R², Y is NR³, R^{E} is methyl, and R^{E} is hydrogen. In some embodiments, n is 1, X is NR¹R², Y is NR³, and R^{E} and R^{F} are methyl. In some embodiments, n is 1, X is NR¹R², Y is NR³, and R^{A}, R^{B}, R^{C}, R^{D}, R^{E}, and R^{F} are hydrogen. In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃⁺, and R² is hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

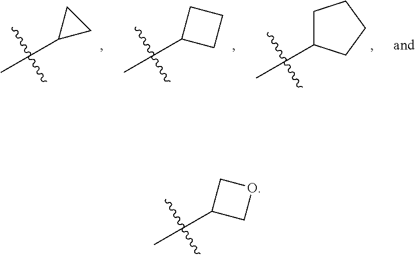

In some embodiments, R³ is

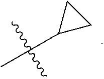 [SMILES: *C1CC1]

In some embodiments, R³ is

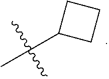 [SMILES: *C1CCC1]

In some embodiments, R³ is

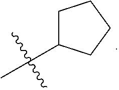 [SMILES: *C1CCCC1]

In some embodiments, R³ is

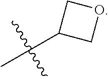 [SMILES: *C1COC1]

In some embodiments, the

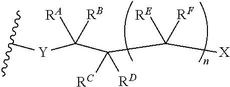

moiety in the prodrugs of Group I is selected from 3

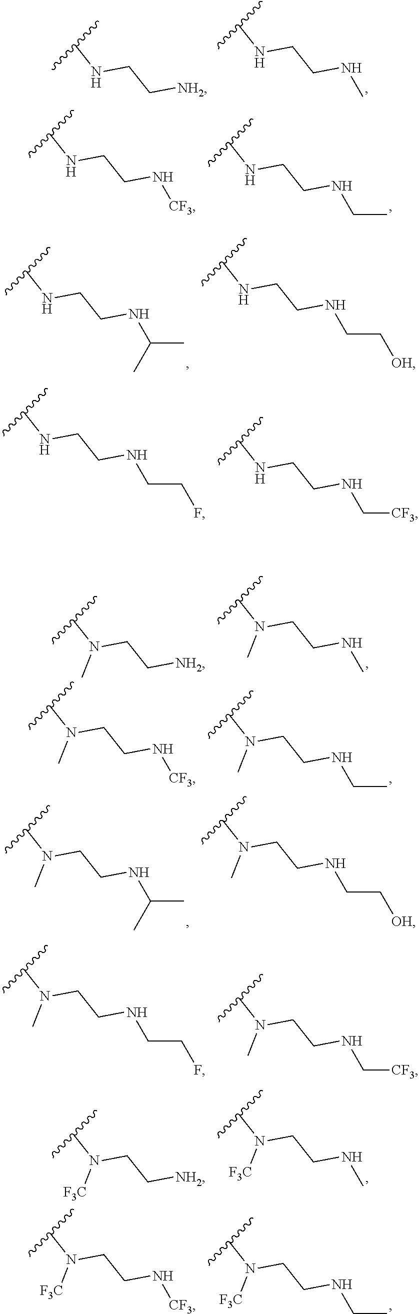

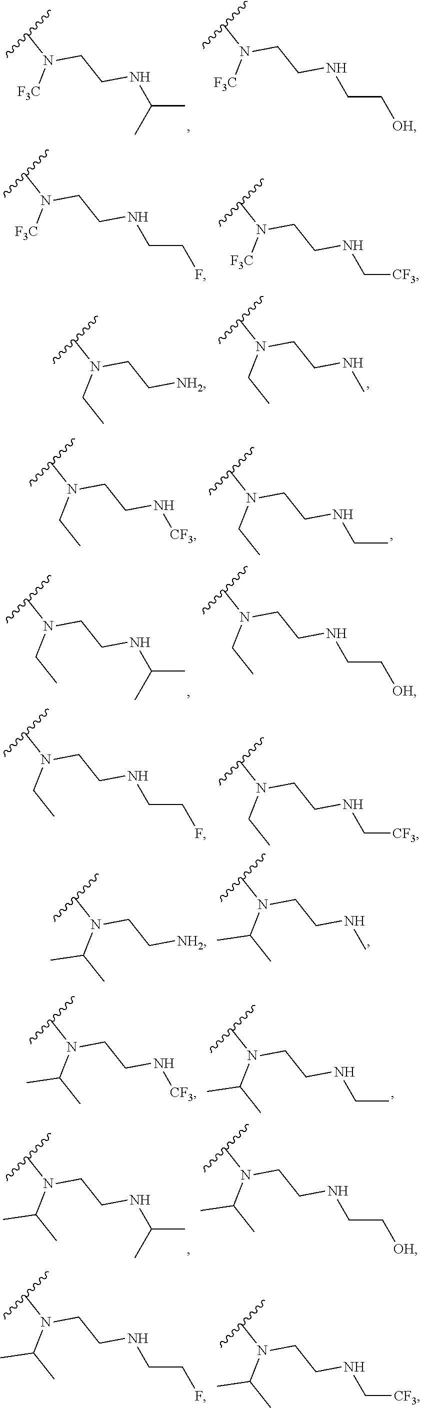

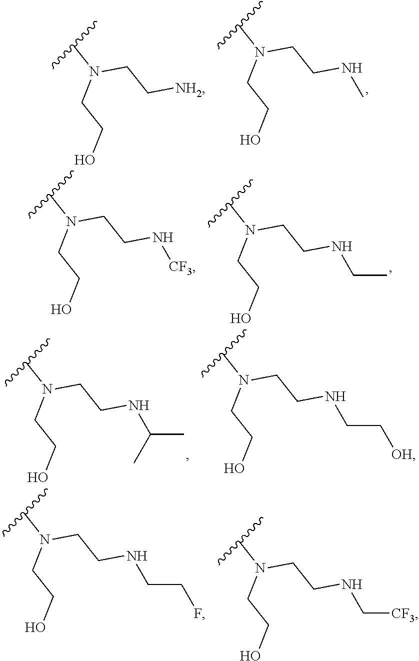

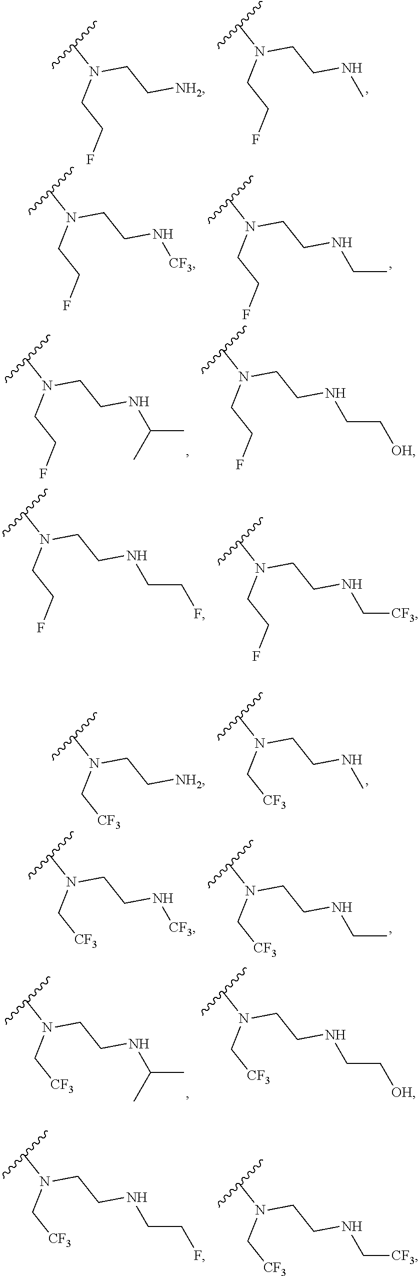

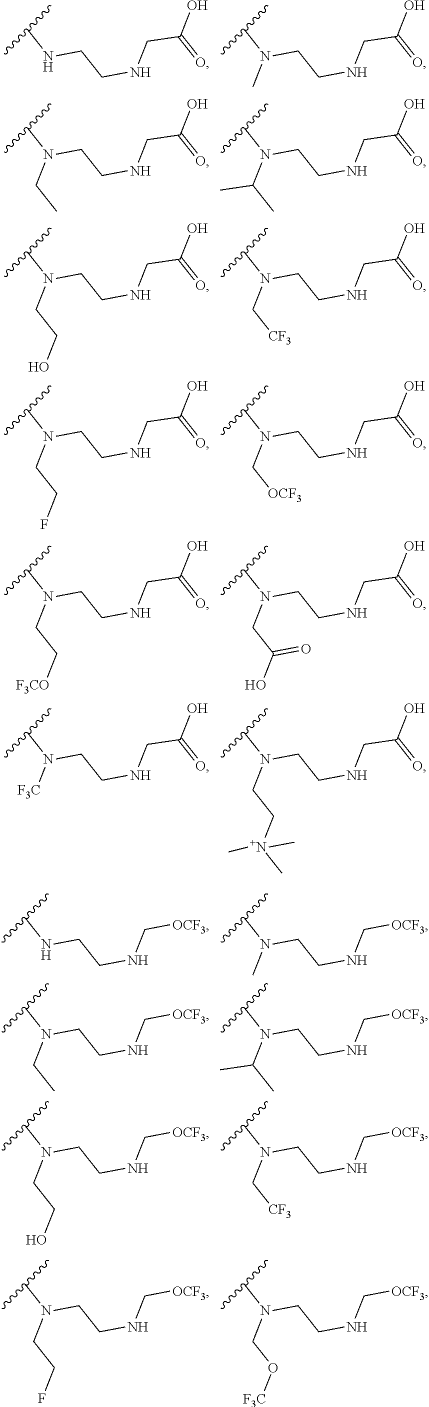

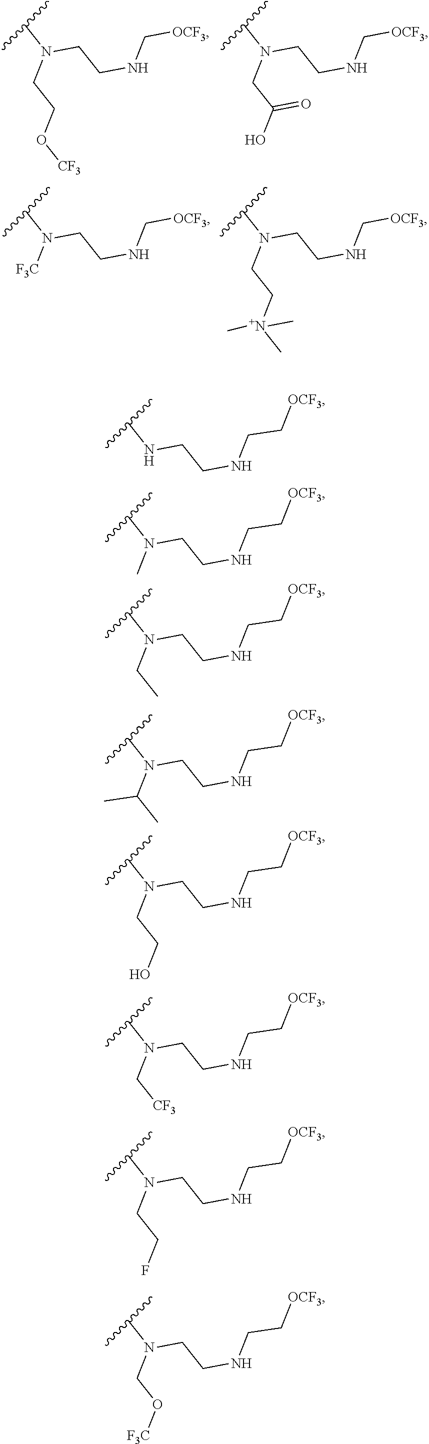

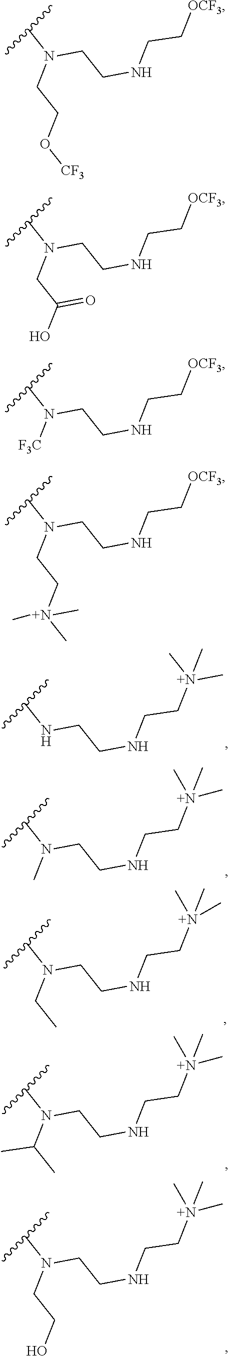

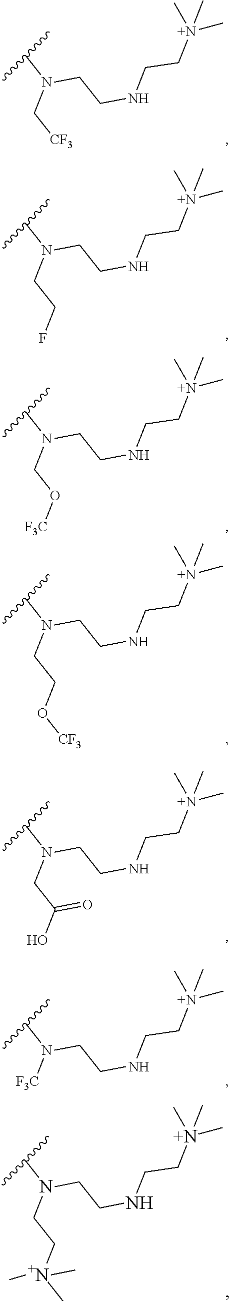

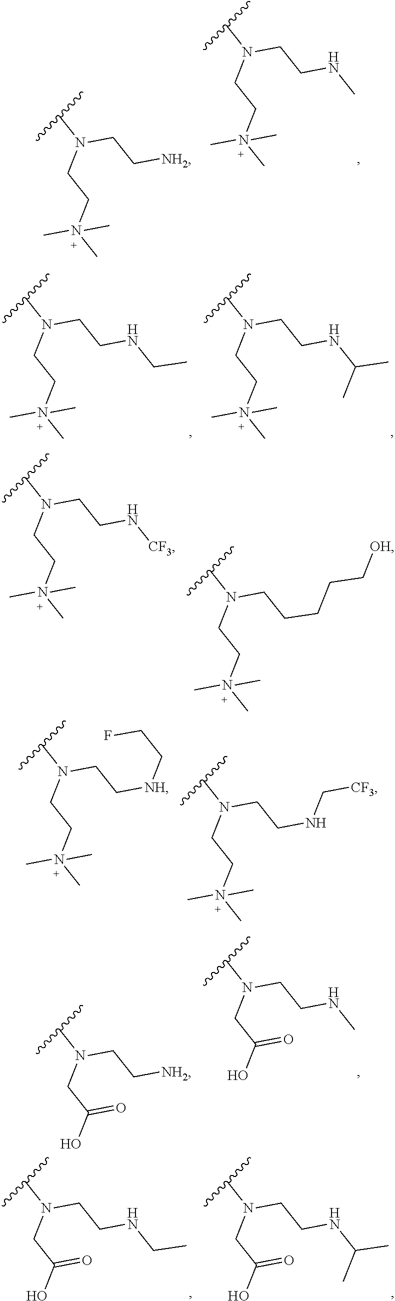

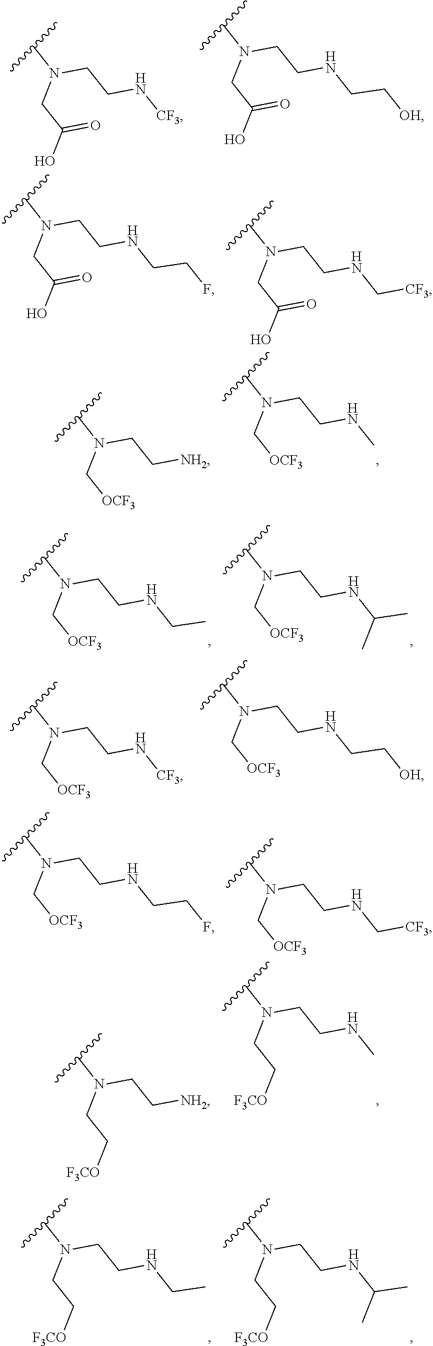

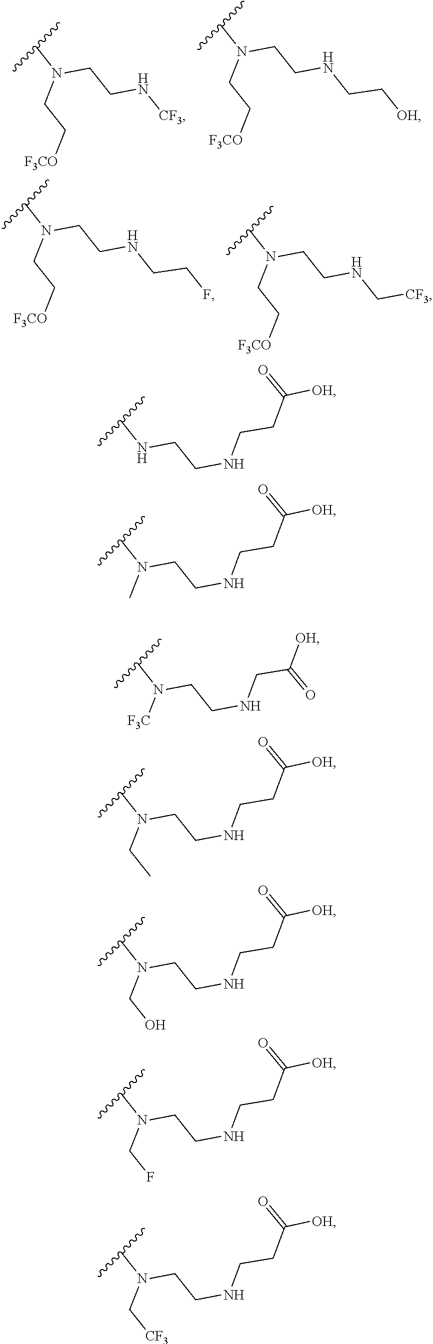

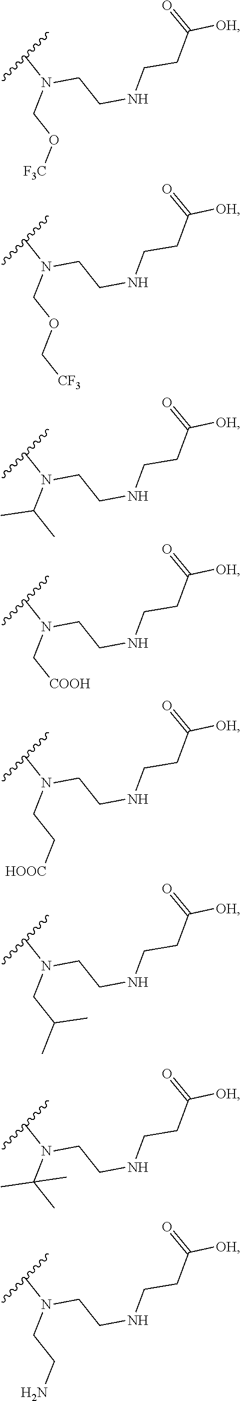

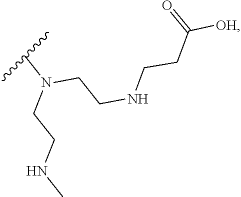 [SMILES: *N(CCNC)CCNCCC(=O)O]

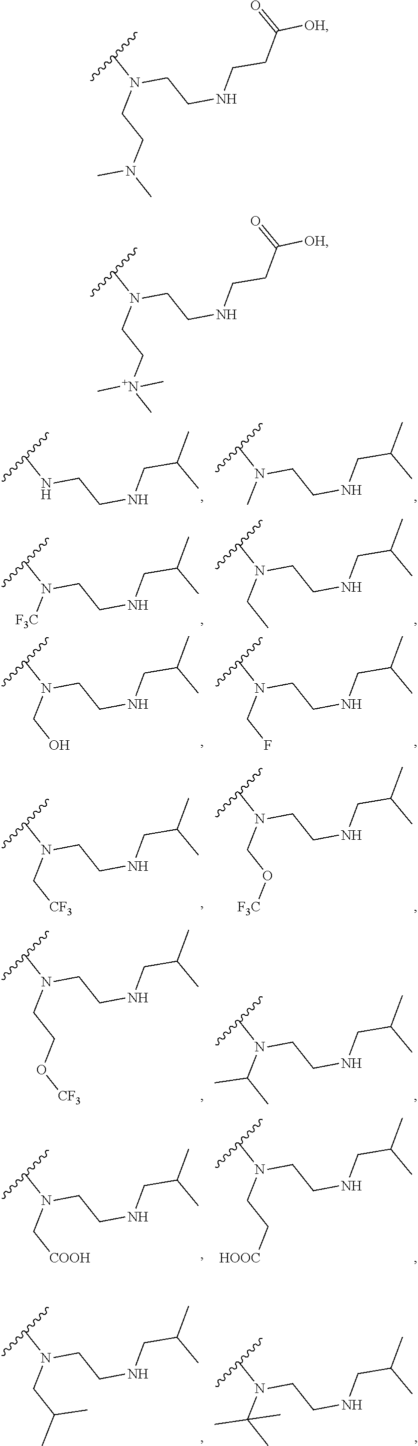

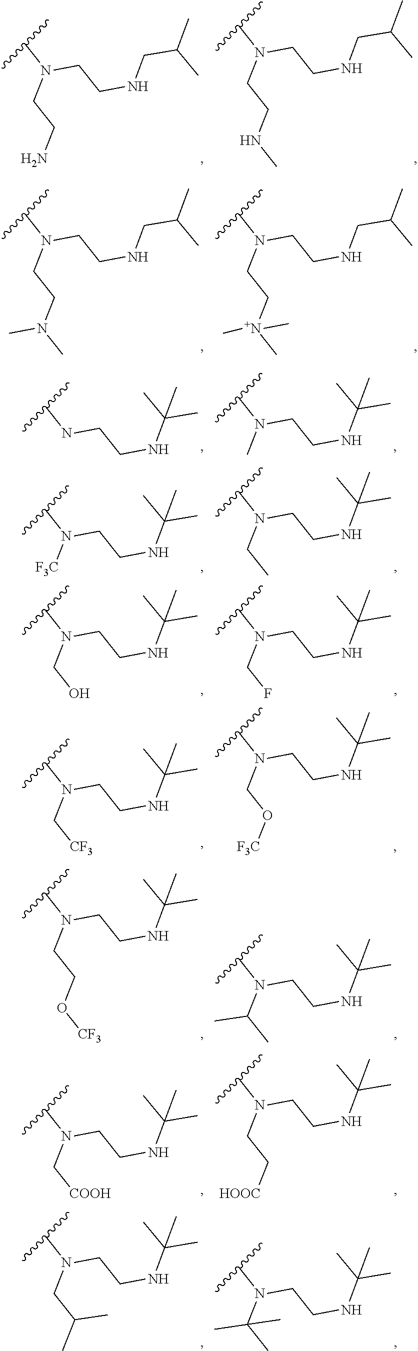

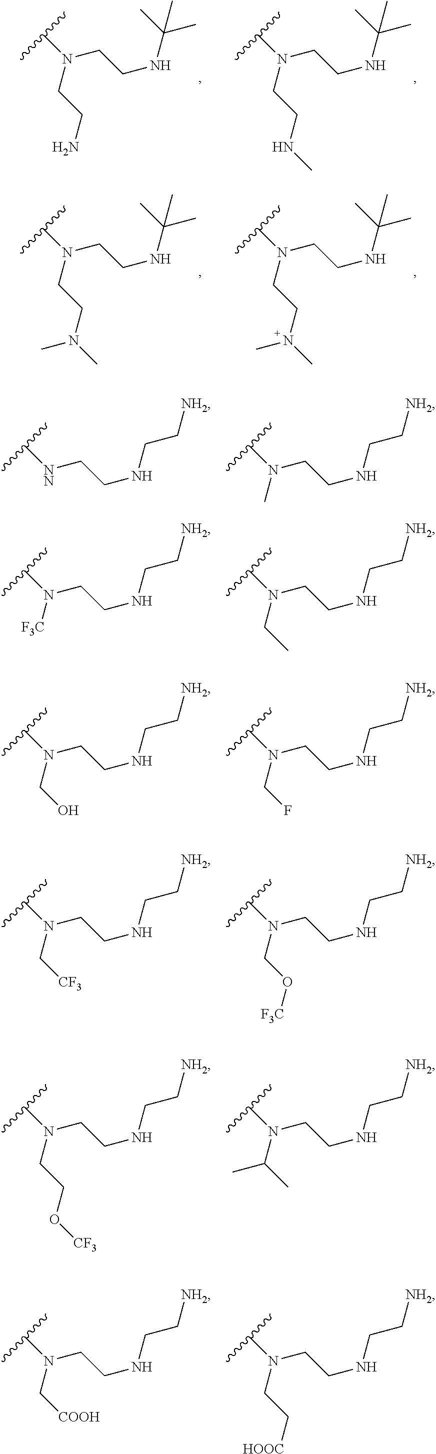

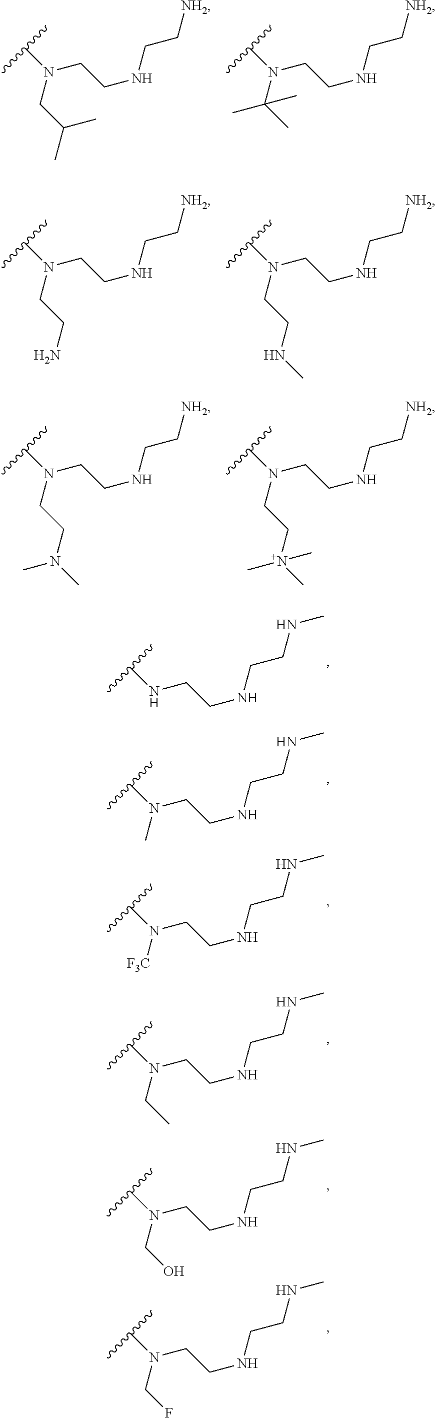

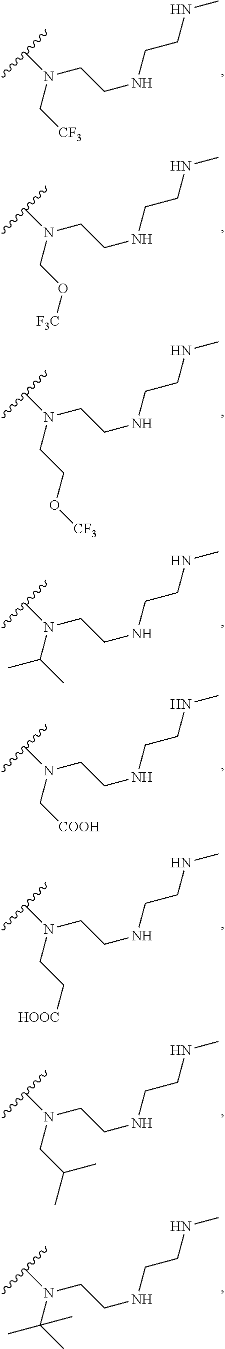

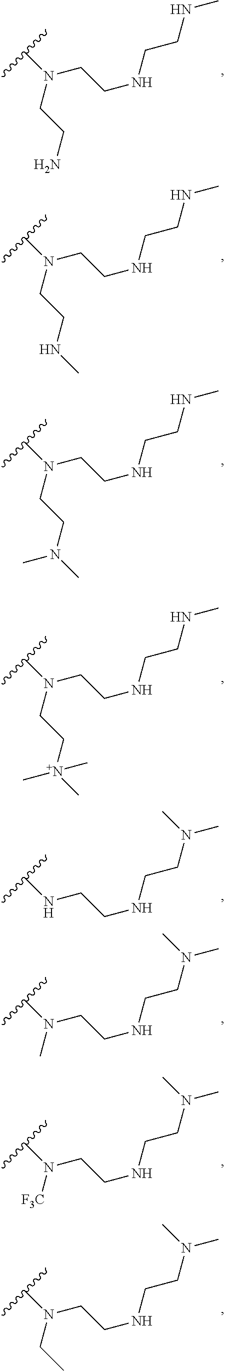

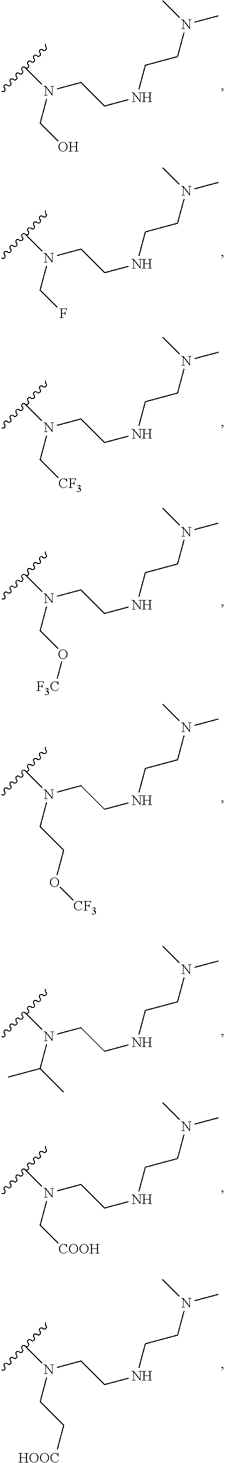

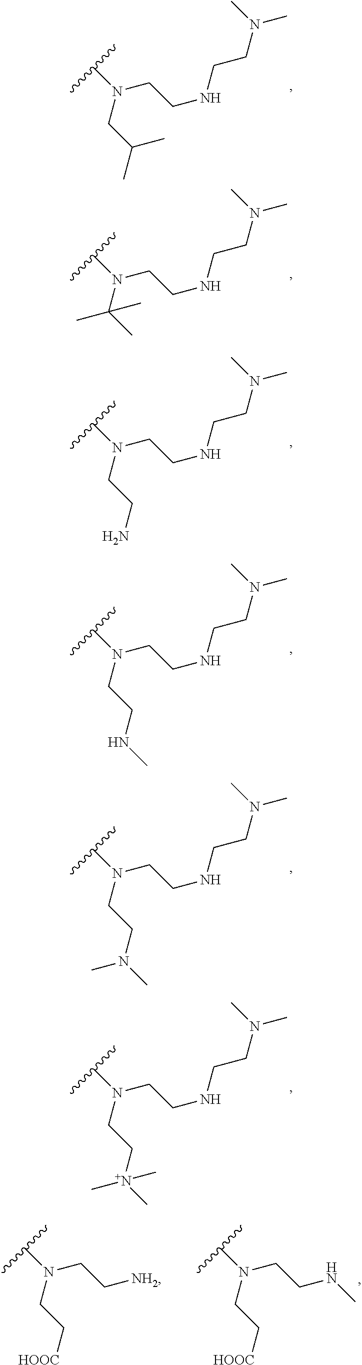

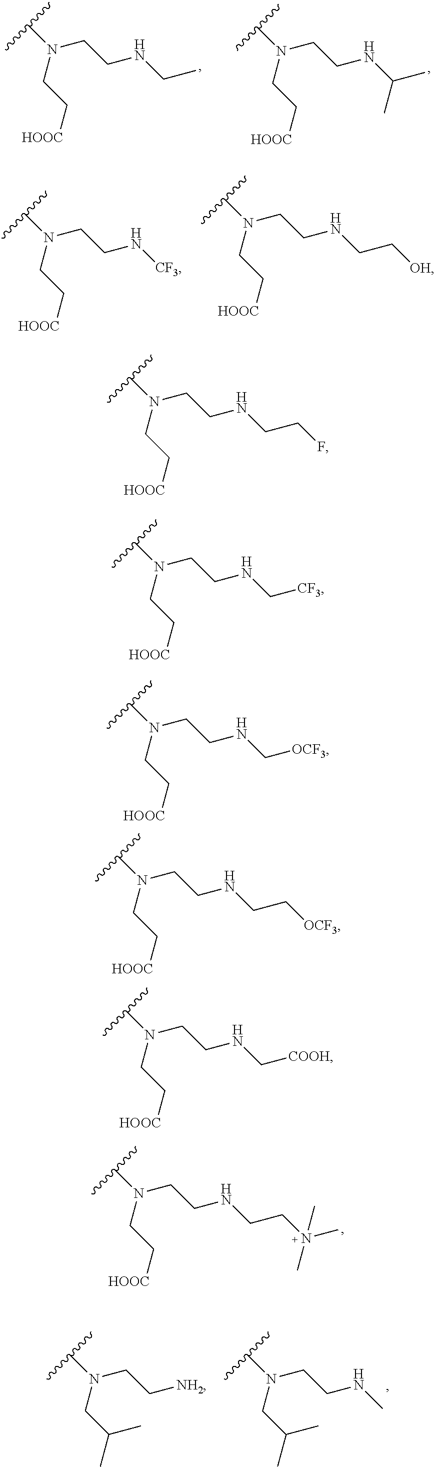

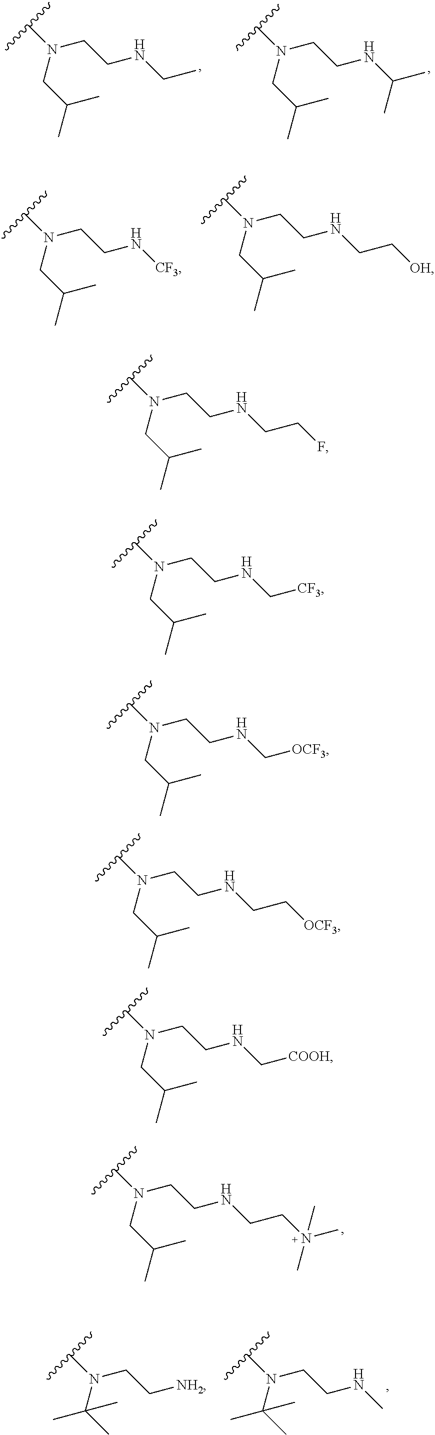

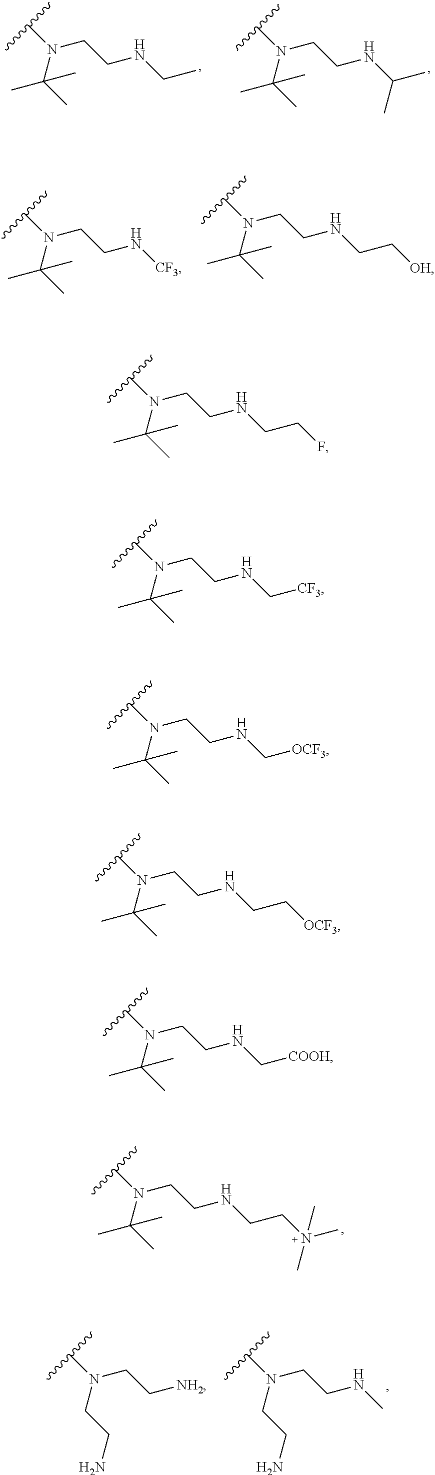

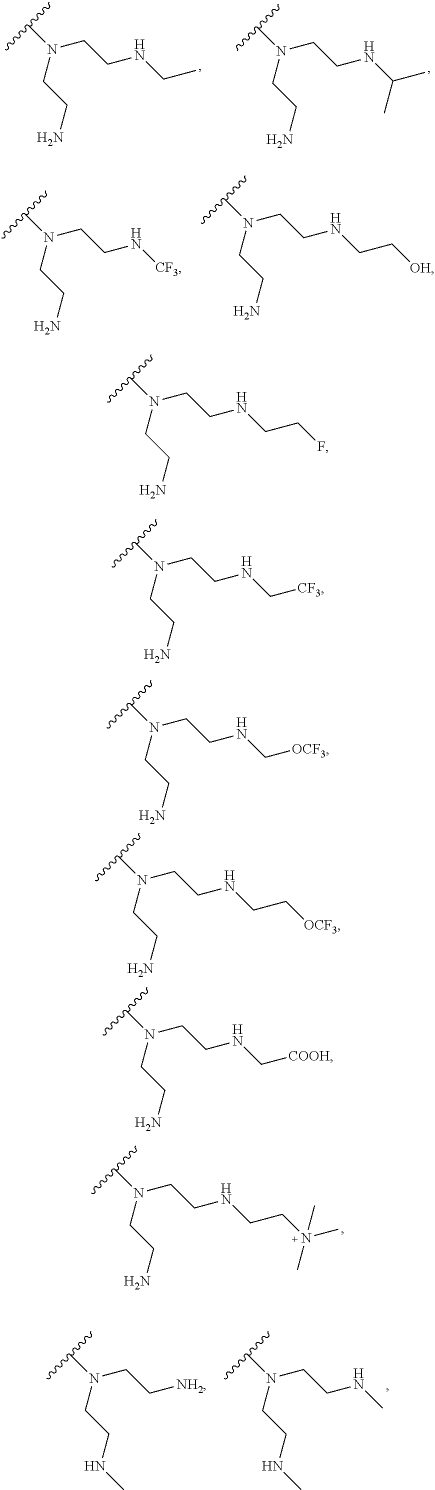

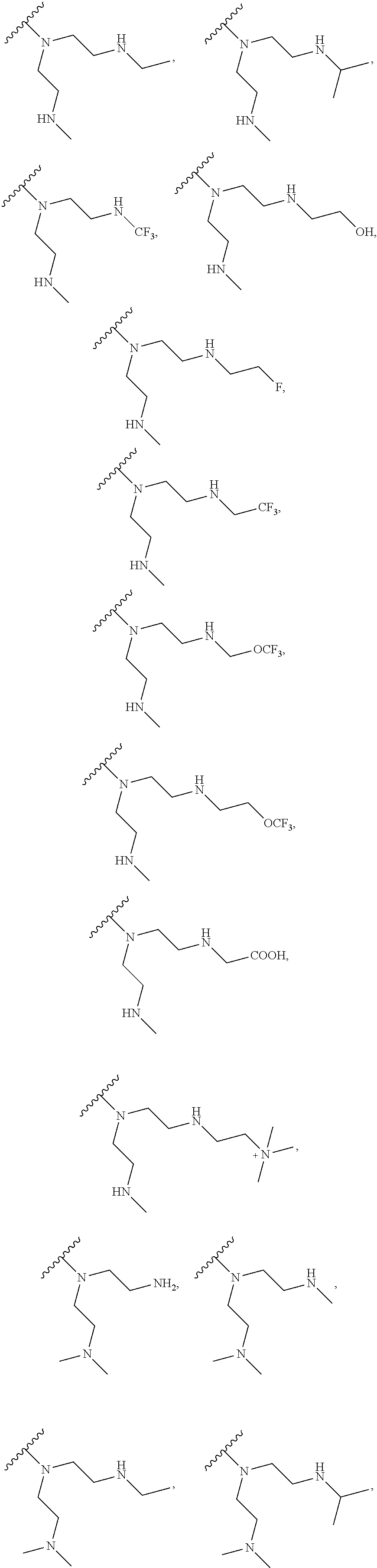

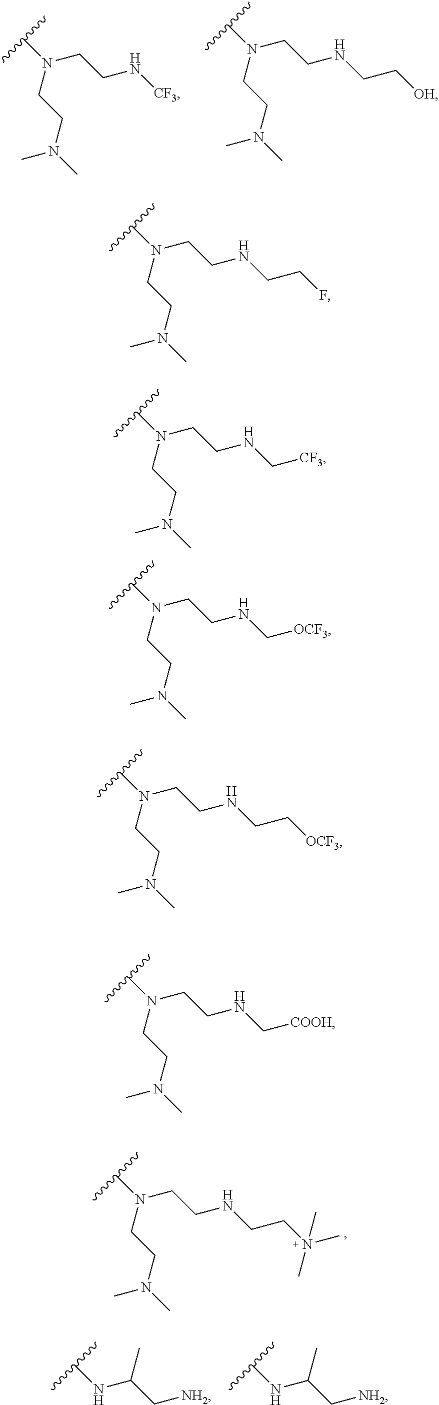

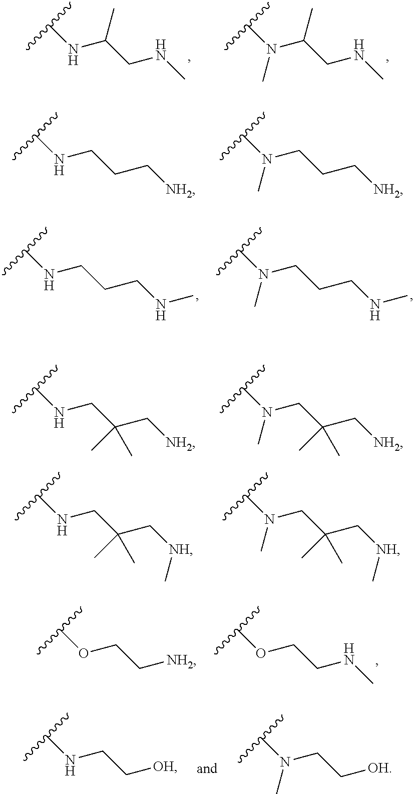

In some embodiments, the

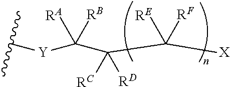

moiety in the prodrugs of Group I is selected from:

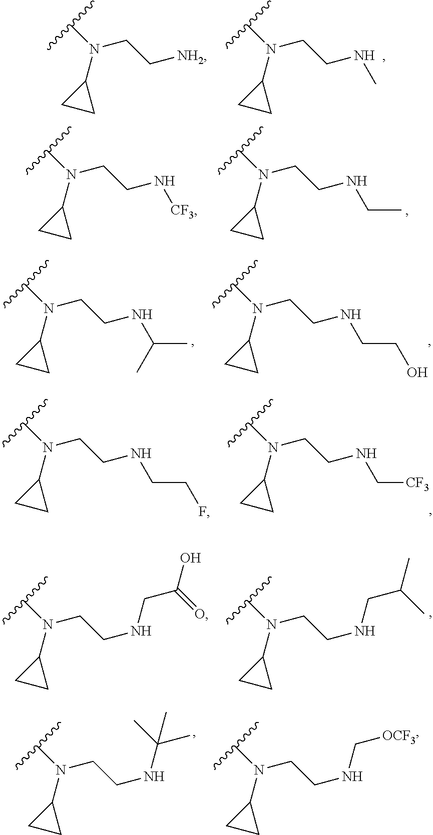

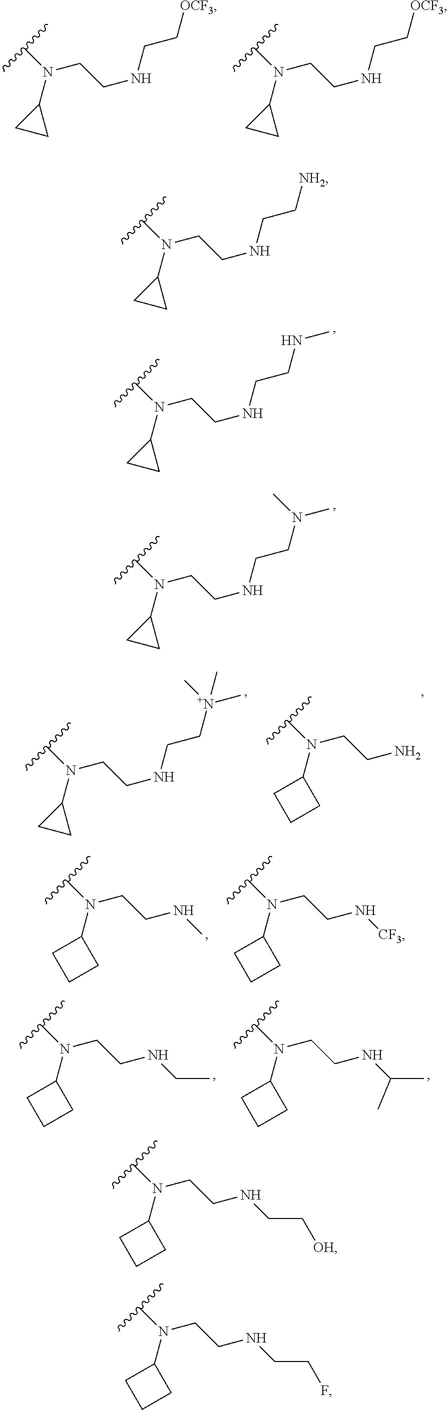

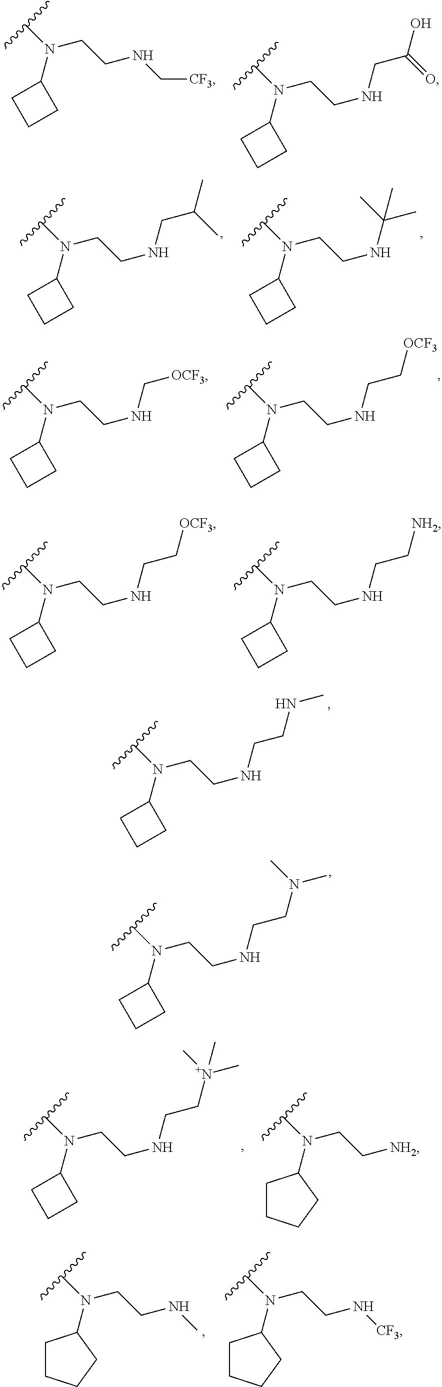

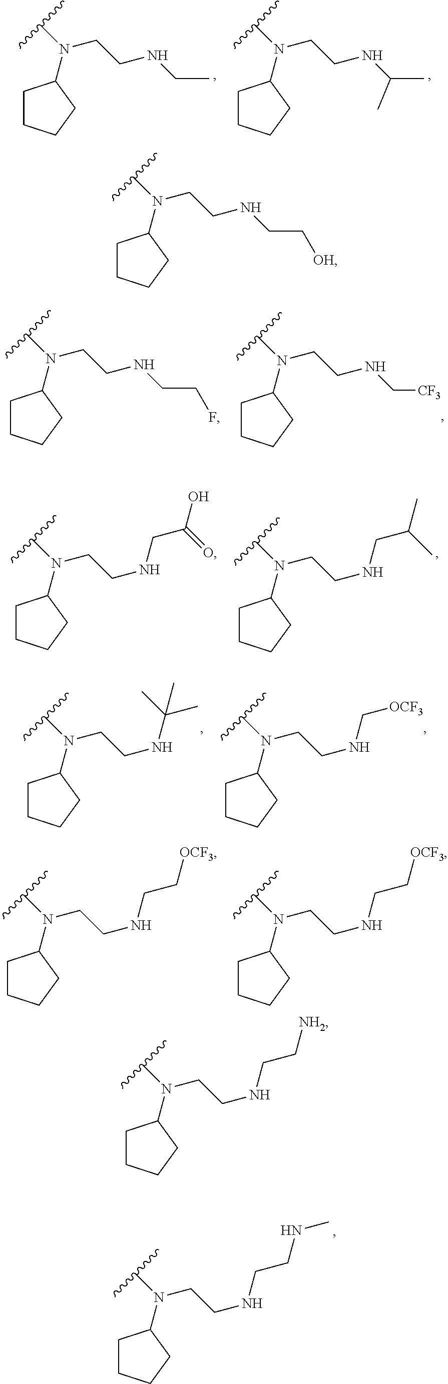

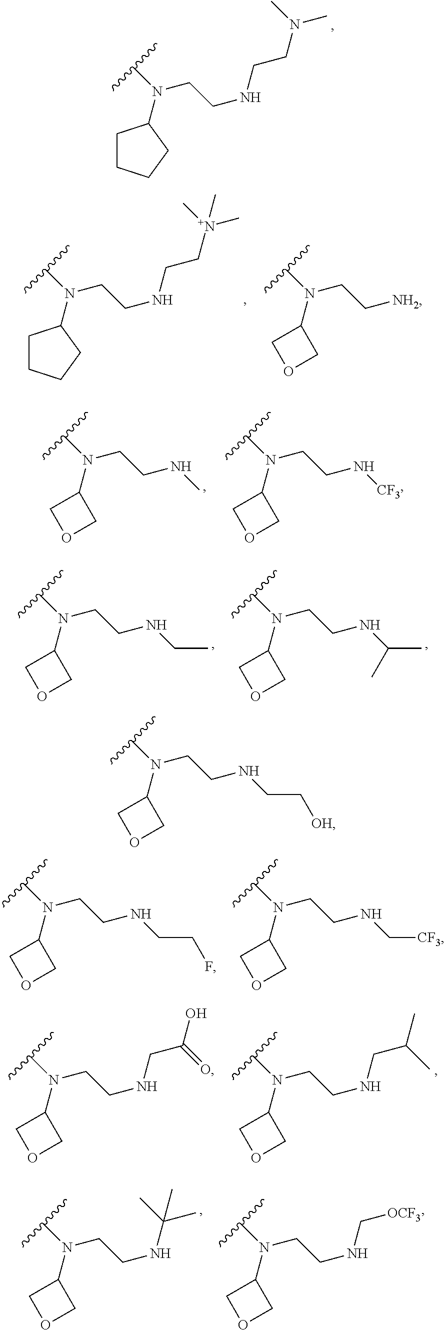

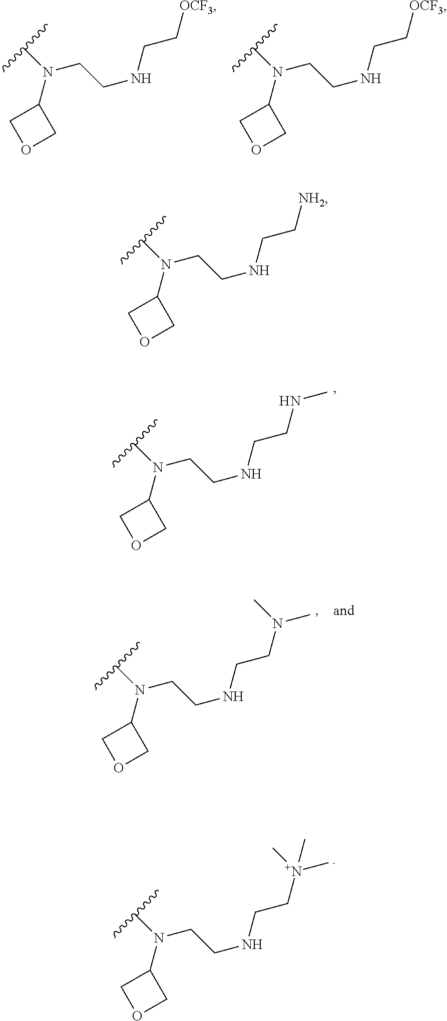 [SMILES: *N(CCNCCOC(F)(F)F)C1COC1 ; *N(CCNCCOC(F)(F)F)C1COC1 ; *N(CCNCCN)C1COC1 ; *N(CCNCCNC)C1COC1 ; *N(CCNCCN(C)C)C1COC1 ; *N(CCNCC[N+](C)(C)C)C1COC1]

In some embodiments, the

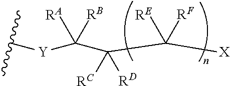

moiety in the prodrugs of Group I is selected from:

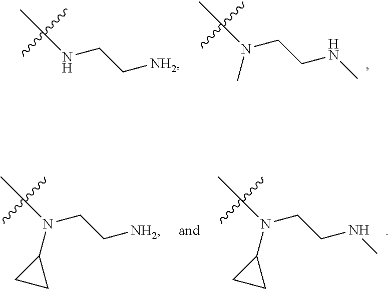

In some embodiments, the

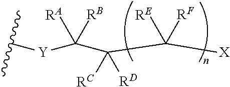

moiety in the prodrugs of Group I is selected is

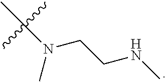 [SMILES: *N(C)CCNC]

### Group II

In some embodiments, the prodrugs have the following features:
X is NR¹R²,
Y is O or NR³,
R¹ joins R^{C} or R^{E} to form a 4-7 membered, optionally substituted heterocycle,
R² is hydrogen,
R^{A}, R^{B}, R^{C}, R^{D}, R^{E}, and R^{F}, on each occurrence when not joined by R¹ to form the heterocycle, are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R³ is independently selected from hydrogen, optionally substituted alkyl, optionally substituted heteroalkyl, optionally substituted carbocyclyl, optionally substituted heterocyclyl, optionally substituted aryl, and optionally substituted heteroaryl.

In some embodiments, the 4-7 membered, optionally substituted heterocycle formed by R¹ joining R^{C} or R^{E} is unsubstituted.

In some embodiments, the 4-7 membered, optionally substituted heterocycle formed by R¹ joining R^{C} or R^{E} is substituted. Suitable substituents are in accordance with the general description of substitution in previous sections. In some embodiments, the 4-7 membered, optionally substituted heterocycle is substituted by one or more substituents independently selected from halogen, hydroxyl, ═O (counting as two substituents), ═S (counting as two substituents), cyano, nitro, carboxyl, carbonate, alkyl, haloalkyl, carbocyclyl, halocarbocyclyl, heterocyclyl, haloheterocyclyl, aryl, haloaryl, acyl, sulfinyl, sulfonyl, sulfonate, sulfonamide, amide, optionally Si-substituted silyl, ester, thioester, carbonate ester, and carbamate. In some embodiments, the 4-7 membered, optionally substituted heterocycle is substituted by one or more substituents independently selected from halogen, hydroxyl, cyano, alkyl, polyfluoroalkyl, ═O, ═S, amide, ester, sulfinyl, sulfonyl, sulfonate, and sulfonamide. In some embodiments, the 4-7 membered, optionally substituted heterocycle is substituted by one or more substituents independently selected from halogen, hydroxyl, ═O, ═S, amide, ester, alkyl, and polyfluoroalkyl. In some embodiments, the 4-7 membered, optionally substituted heterocycle is substituted by one or more substituents independently selected from fluorine, hydroxyl, ═O, ═S, —C(═O)NH₂, —C(═O)NHCH₃, —C(═O)N(CH₃)₂, —C(═O)OCH(CH₃)₂, methyl, and trifluoromethyl. In some embodiments, the 4-7 membered, optionally substituted heterocycle is substituted by one or more fluorine atoms. In some embodiments, the 4-7 membered, optionally substituted heterocycle is substituted by a —C(═O)NH₂ group. In some embodiments, the 4-7 membered, optionally substituted heterocycle is substituted by a —C(═O)OCH(CH₃)₂ group. In some embodiments, the 4-7 membered, optionally substituted heterocycle is substituted by a ═O group.

In some embodiments, the 4-7 membered, optionally substituted heterocycle is a 5 or 6 membered, optionally substituted heterocycle. In some embodiments, the 4-7 membered, optionally substituted heterocycle is a 5-membered, optionally substituted heterocycle. In some embodiments, the 4-7 membered, optionally substituted heterocycle is a 6-membered, optionally substituted heterocycle. In some embodiments, the 4-7 membered, optionally substituted heterocycle is optionally substituted pyrrolidine or optionally substituted piperidine. In some embodiments, the 4-7 membered, optionally substituted heterocycle is optionally substituted pyrrolidine. In some embodiments, the 4-7 membered, optionally substituted heterocycle is pyrrolidine or piperidine. In some embodiments, the 4-7 membered, optionally substituted heterocycle is pyrrolidine.

In some embodiments, Y is O. In some embodiments, Y is NR³. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

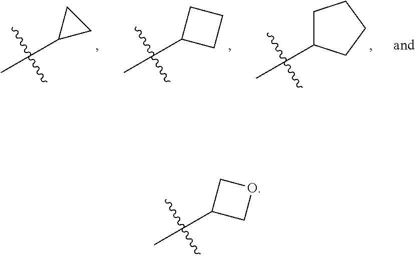

In some embodiments, R³ is

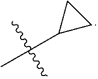 [SMILES: *C1CC1]

In some embodiments, R³ is

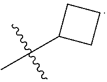 [SMILES: *C1CCC1]

In some embodiments, R³ is

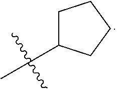 [SMILES: *C1CCCC1]

In some embodiments, R³ is

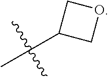 [SMILES: *C1COC1]

Optionally, at least one of R^{A} and R^{B} is hydrogen. In some embodiments, both R^{A} and R^{B} are hydrogen. In some embodiments, R^{A} is optionally substituted alkyl, and R^{B} is hydrogen. In some embodiments, R^{A} is optionally substituted C₁-C₄ alkyl, and R^{B} is hydrogen. In some embodiments, R^{A} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{B} is hydrogen. In some embodiments, R^{A} is methyl, and R^{B} is hydrogen.

Optionally, at least one of R^{A} and R^{B} is optionally substituted alkyl. In some embodiments, at least one of R^{A} and R^{B} is optionally substituted C₁-C₄ alkyl. In some embodiments, at least one of R^{A} and R^{B} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{A} and R^{B} is methyl. In some embodiments, each of R^{A} and R^{B} is, independently, an optionally substituted alkyl. In some embodiments, each of R^{A} and R^{B} is, independently, an optionally substituted C₁-C₄ alkyl. In some embodiments, each of R^{A} and R^{B} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{A} and R^{B} is methyl.

In some embodiments, n is 0, i.e., both R^{E} and R^{F} are absent. When n is 0, R¹ can only join Ru to form the 4-7 membered, optionally substituted heterocycle.

In some embodiments, n is 1, i.e., both R^{E} and R^{F} are present. When n is 1, R¹ can join either R^{C} or R^{E} to form the 4-7 membered, optionally substituted heterocycle.

In some embodiments, R¹ joins R^{C} to form the 4-7 membered, optionally substituted heterocycle. When R¹ joins R^{C} to form the 4-7 membered, optionally substituted heterocycle, n can be 0 or 1.

In some embodiments, R¹ joins R^{E} to form the 4-7 membered, optionally substituted heterocycle. When R¹ joins R^{E} to form the 4-7 membered, optionally substituted heterocycle, n can only be 1.

Optionally, when R¹ joins R^{C} to form the 4-7 membered, optionally substituted heterocycle, R^{D} is hydrogen.

Optionally, when R¹ joins R^{C} to form the 4-7 membered, optionally substituted heterocycle, R^{D} is optionally substituted alkyl. In some embodiments, R^{D} is optionally substituted C₁-C₄ alkyl.

In some embodiments, R^{D} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R^{D} is methyl.

Optionally, when R¹ joins R^{C} to form the 4-7 membered, optionally substituted heterocycle and n is 0, both R^{E} and R^{F} are absent.

Optionally, when R¹ joins R^{C} to form the 4-7 membered, optionally substituted heterocycle and n is 1, at least one of R^{E} and R^{F} is hydrogen. In some embodiments, both R^{E} and R^{F} are hydrogen. In some embodiments, R^{E} is optionally substituted alkyl, and R^{F} is hydrogen. In some embodiments, R^{E} is optionally substituted C₁-C₄ alkyl, and R^{F} is hydrogen. In some embodiments, R^{E} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃, and R^{F} is hydrogen. In some embodiments, R^{E} is methyl, and R^{F} is hydrogen.

Optionally, when R¹ joins R^{C} to form the 4-7 membered, optionally substituted heterocycle and n is 1, at least one of R^{E} and R^{F} is optionally substituted alkyl. In some embodiments, at least one of R^{E} and R^{F} is optionally substituted C₁-C₄ alkyl. In some embodiments, at least one of R^{E} and R^{F} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{E} and R^{F} is methyl. In some embodiments, each of R^{E} and R^{F} is, independently, an optionally substituted alkyl. In some embodiments, each of R^{E} and R^{F} is, independently, an optionally substituted C₁-C₄ alkyl. In some embodiments, each of R^{E} and R^{F} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{E} and R^{F} is methyl.

Optionally, when R¹ joins R^{E} to form the 4-7 membered, optionally substituted heterocycle, R^{F} is hydrogen.

Optionally, when R¹ joins R^{E} to form the 4-7 membered, optionally substituted heterocycle, R^{F} is optionally substituted alkyl. In some embodiments, R^{F} is optionally substituted C₁-C₄ alkyl. In some embodiments, R^{F} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R^{F} is methyl.

Optionally, when R¹ joins R^{E} to form the 4-7 membered, optionally substituted heterocycle, at least one of R^{C} and R^{D} is hydrogen. In some embodiments, both R^{C} and R^{D} are hydrogen. In some embodiments, R^{e} is optionally substituted alkyl, and R^{D} is hydrogen. In some embodiments, R^{C} is optionally substituted C₁-C₄ alkyl, and R^{D} is hydrogen. In some embodiments, R^{C} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{D} is hydrogen. In some embodiments, R^{C} is methyl, and R^{D} is hydrogen.

Optionally, when R¹ joins R^{E} to form the 4-7 membered, optionally substituted heterocycle, at least one of R^{C} and R^{D} is optionally substituted alkyl. In some embodiments, at least one of R^{C} and R^{D} is optionally substituted C₁-C₄ alkyl. In some embodiments, at least one of R^{C} and R^{D} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{C} and R^{D} is methyl. In some embodiments, each of R^{C} and R^{D} is, independently, an optionally substituted alkyl. In some embodiments, each of R^{C} and R^{D} is, independently, an optionally substituted C₁-C₄ alkyl. In some embodiments, each of R^{C} and R^{D} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{C} and R^{D} is methyl.

In some embodiments, when R¹ joins R^{C} to form the 4-7 membered, optionally substituted heterocycle, R^{A}, R^{B}, and R^{D} are hydrogen. In some embodiments, when R¹ joins R^{C} to form the 4-7 membered, optionally substituted heterocycle and n is 0, R^{A}, R^{B}, and R^{D} are hydrogen. In some embodiments, when R¹ joins R^{C} to form the 4-7 membered, optionally substituted heterocycle and n is 1, R^{A}, R^{B}, and R^{D} are hydrogen. In some embodiments, when R¹ joins R^{C} to form the 4-7 membered, optionally substituted heterocycle and n is 1, R^{A}, R^{B}, R^{D}, R^{E}, and R^{F} are hydrogen.

In some embodiments, when R¹ joins R^{E} to form the 4-7 membered, optionally substituted heterocycle, R^{A}, R^{B}, R^{C}, and R^{D} are hydrogen. In some embodiments, when R¹ joins R^{E} to form the 4-7 membered, optionally substituted heterocycle, R^{A}, R^{B}, R^{C}, R^{D}, and R^{F} are hydrogen.

In some embodiments, n is 0, and Y is NR³. In some embodiments, n is 0, Y is NR³, and R^{A} and R^{B} are hydrogen. In some embodiments, n is 0, Y is NR³, R^{A} is methyl, and R^{B} is hydrogen. In some embodiments, n is 0, Y is NR³, and R^{A} and R^{B} are methyl. In some embodiments, n is 0, Y is NR³, and R^{D} is hydrogen. In some embodiments, n is 0, Y is NR³, and R^{D} is methyl. In some embodiments, n is 0, Y is NR³, and R^{A}, R^{B}, and R^{D} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

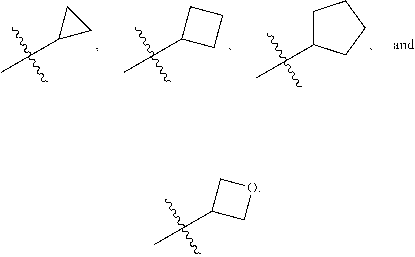

In some embodiments, R³ is

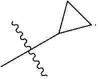 [SMILES: *C1CC1]

In some embodiments, R³ is

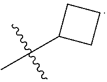 [SMILES: *C1CCC1]

In some embodiments, R³ is

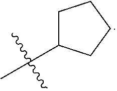 [SMILES: *C1CCCC1]

In some embodiments, R³ is

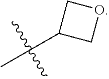 [SMILES: *C1COC1]

In some embodiments, n is 1, and Y is NR³.

In some embodiments, n is 1, Y is NR³, and R¹ joins R^{e} to form the 4-7 membered, optionally substituted heterocycle. In some embodiments, n is 1, Y is NR³, R¹ joins R^{C} to form the 4-7 membered, optionally substituted heterocycle, and R^{A} and R^{B} are hydrogen. In some embodiments, n is 1, Y is NR³, R¹ joins R^{C} to form the 4-7 membered, optionally substituted heterocycle, R^{A} is methyl, and R^{B} is hydrogen. In some embodiments, n is 1, Y is NR³, R¹ joins R^{C} to form the 4-7 membered, optionally substituted heterocycle, and R^{A} and R^{B} are methyl. In some embodiments, n is 1, Y is NR³, R¹ joins R^{C} to form the 4-7 membered, optionally substituted heterocycle, and R^{D} is hydrogen. In some embodiments, n is 1, Y is NR³, R¹ joins R^{C} to form the 4-7 membered, optionally substituted heterocycle, and R^{D} is methyl. In some embodiments, n is 1, Y is NR³, R¹ joins R^{C} to form the 4-7 membered, optionally substituted heterocycle, and R^{E} and R^{F} are hydrogen. In some embodiments, n is 1, Y is NR³, R¹ joins R^{C} to form the 4-7 membered, optionally substituted heterocycle, R^{E} is methyl, and R^{F} is hydrogen. In some embodiments, n is 1, Y is NR³, R¹ joins R^{C} to form the 4-7 membered, optionally substituted heterocycle, and R^{E} and R^{F} are methyl. In some embodiments, n is 1, Y is NR³, R¹ joins R^{C} to form the 4-7 membered, optionally substituted heterocycle, and R^{A}, R^{B}, R^{D}, R^{E}, and R^{F} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

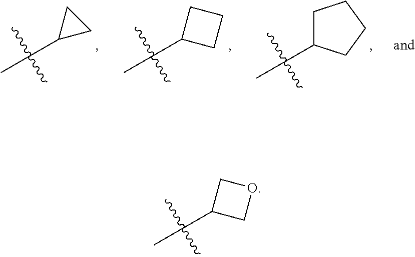

In some embodiments, R³ is

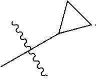 [SMILES: *C1CC1]

In some embodiments, R³ is

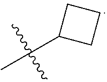 [SMILES: *C1CCC1]

In some embodiments, R³ is

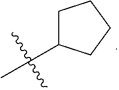 [SMILES: *C1CCCC1]

In some embodiments, R³ is

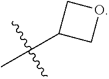 [SMILES: *C1COC1]

In some embodiments, n is 1, Y is NR³, and R¹ joins R^{E} to form the 4-7 membered, optionally substituted heterocycle. In some embodiments, n is 1, Y is NR³, R¹ joins R^{E} to form the 4-7 membered, optionally substituted heterocycle, and R^{A} and R^{B} are hydrogen. In some embodiments, n is 1, Y is NR³, R¹ joins R^{E} to form the 4-7 membered, optionally substituted heterocycle, R^{A} is methyl, and R^{B} is hydrogen. In some embodiments, n is 1, Y is NR³, R¹ joins R^{E} to form the 4-7 membered, optionally substituted heterocycle, and R^{A} and R^{B} are methyl. In some embodiments, n is 1, Y is NR³, R¹ joins R^{E} to form the 4-7 membered, optionally substituted heterocycle, and R^{C} and R^{D} are hydrogen. In some embodiments, n is 1, Y is NR³, R¹ joins R^{E} to form the 4-7 membered, optionally substituted heterocycle, R^{C} is methyl, and R^{D} is hydrogen. In some embodiments, n is 1, Y is NR³, R¹ joins R^{E} to form the 4-7 membered, optionally substituted heterocycle, and R^{C} and R^{D} are methyl. In some embodiments, n is 1, Y is NR³, R¹ joins R^{E} to form the 4-7 membered, optionally substituted heterocycle, and R^{F} is hydrogen. In some embodiments, n is 1, Y is NR³, R¹ joins R^{E} to form the 4-7 membered, optionally substituted heterocycle, and R^{F} is methyl. In some embodiments, n is 1, Y is NR³, R¹ joins R^{E} to form the 4-7 membered, optionally substituted heterocycle, and R^{A}, R^{B}, R^{C}, R^{D}, and R^{F} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

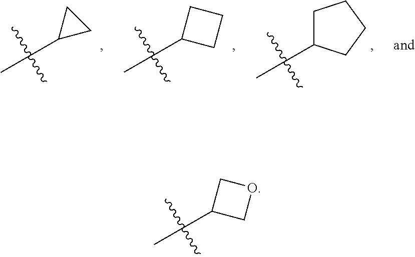

In some embodiments, R³ is

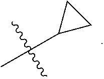 [SMILES: *C1CC1]

In some embodiments, R³ is

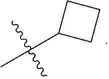 [SMILES: *C1CCC1]

In some embodiments, R³ is

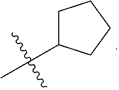 [SMILES: *C1CCCC1]

In some embodiments, R³is

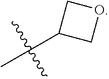 [SMILES: *C1COC1]

In some embodiments, Formula II is in the form of Formula IIC,

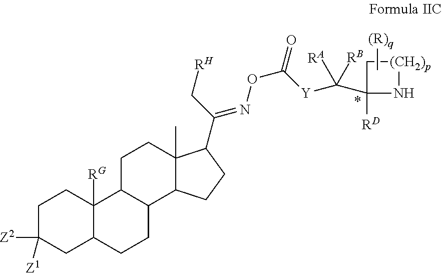

wherein Y, R^{A}, R^{H}, and R^{D} are the same as those described above in this section (i.e., Group II),
wherein p is an integer selected from 1-4,
wherein q is an integer selected from 0-6, and
wherein R is the same as those described above.

In some embodiments, the prodrugs are in a non-salt form as shown in Formula IIC. In some embodiments, the prodrugs are in a salt form. In some embodiments, the prodrugs are in an HCl salt form.

In some embodiments, Formula IIC is in the form of Formula IIC-1. In some embodiments, the prodrugs are in a non-salt form as shown in Formula IIC-1. In some embodiments, the prodrugs are in a salt form. In some embodiments, the prodrugs are in an HCl salt form.

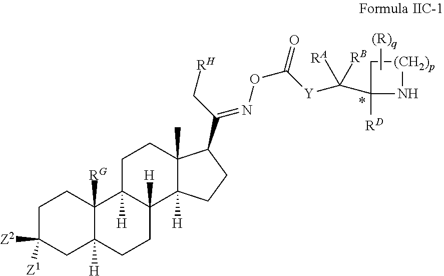

In some embodiments, Formula IIC is in the form of Formula IIC-2. In some embodiments, the prodrugs are in a non-salt form as shown in Formula IIC-2. In some embodiments, the prodrugs are in a salt form. In some embodiments, the prodrugs are in an HCl salt form.

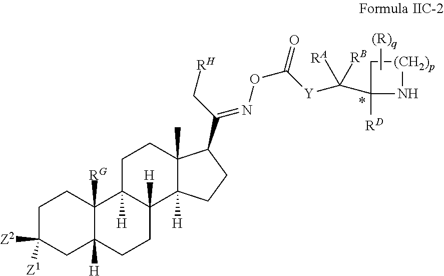

In some embodiments, the carbon atom labeled by the “*” sign is in a S configuration. In some embodiments, the carbon atom labeled by the “*” sign is in a R configuration.

In some embodiments, p is 1, 2, or 3. In some embodiments, p is 1. In some embodiments, p is 2. In some embodiments, p is 3.

In some embodiments, q is 0, 1, or 2. In some embodiments, q is 0. In some embodiments, q is 1. In some embodiments, q is 2.

In some embodiments, p is 1, 2, or 3, and q is 0, 1, or 2. In some embodiments, p is 2, and q is 0, 1, or 2. In some embodiments, p is 2, and q is 0. In some embodiments, p is 3, and q is 0, 1, or 2. In some embodiments, p is 3, and q is 0.

In some examples, the R groups are independently selected from halogen, nitro, cyano, hydroxyl, formyl, carboxyl, thiol, ═O (counting as two R groups), ═S (counting two R groups), sulfamoyl, alkyl (such as methyl, ethyl, isopropyl, tert-butyl), haloalkyl (such as trifluoromethyl), alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, arylalkyl (such as benzyl), alkylaryl, alkyloxy (such as methoxy, ethoxy), haloalkyloxy (such as trifluoromethoxy), aryloxy, alkylcarbonyl (such as acetyl), arylcarbonyl (such as benzoyl), alkylcarbonyloxy (such as acetoxy), arylcarbonyloxy (such as benzoyloxy), alkyloxycarbonyl (such as methoxycarbonyl, ethoxycarbonyl, isopropoxycarbonyl), aryloxycarbonyl, primary amino, alkylamino (such as methylamino, ethylamino, dimethylamino, diethylamino, N-methyl-N-ethylamino), alkylammonium (such as trimethylammonium), alkylcarbonylamino (such as acetylamino), arylcarbonylamino (such as benzoylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), and N-alkylsulfamoyl (such as N-methylsulfamoyl, N-ethylsulfamoyl, N,N-dimethylsulfamoyl, N,N-diethylsulfamoyl, N-methyl-N-ethylsulfamoyl).

In some examples, the R groups are independently selected from halogen, nitro, cyano, hydroxyl, trifluoromethyl, methoxy, ethoxy, trifluoromethoxy, primary amino, formyl, carboxyl, carbamoyl, thiol, ═O, ═S, sulfamoyl, acetyl, acetoxy, methoxycarbonyl, ethoxycarbonyl, isopropoxycarbonyl, methylamino, ethylamino, dimethylamino, diethylamino, N-methyl-N-ethylamino, trimethylammonium, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, and haloheteroaryl.

In some examples, the R groups are independently selected from halogen, ═O, ═S, alkyl, haloalkyl, carbocyclyl, halocarbocyclyl, aryl, haloaryl, heterocyclyl, and haloheterocyclyl.

In some examples, the R groups are independently selected from halogen, ═O, ═S, alkyl, haloalkyl, carbocyclyl, halocarbocyclyl, aryl, haloaryl, heterocyclyl, haloheterocyclyl, carbamoyl, N-alkylcarbamoyl, alkyloxycarbonyl, and aryloxycarbonyl.

In some examples, q is an integer selected from 1-6, and one of the R groups is carbamoyl or N-alkylcarbamoyl. For example, q is 1 and R is carbamoyl.

In some examples, q is an integer selected from 1-6, and one of the R groups is alkyloxycarbonyl or aryloxycarbonyl. For example, q is 1 and R is isopropoxycarbonyl.

In some examples, q is an integer selected from 1-6, and one of the R groups is fluorine. For example, q is 2 and both R groups are fluorine (they can be attached to the same atom or two different atoms).

In some examples, q is an integer selected from 2-6, and two of the R groups constitute ═O or ═S. For example, q is 2 and the two R groups constitute ═O.

In some embodiments, the

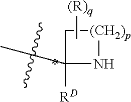

moiety in Formula IIC and its sub-formulas is selected from the following:

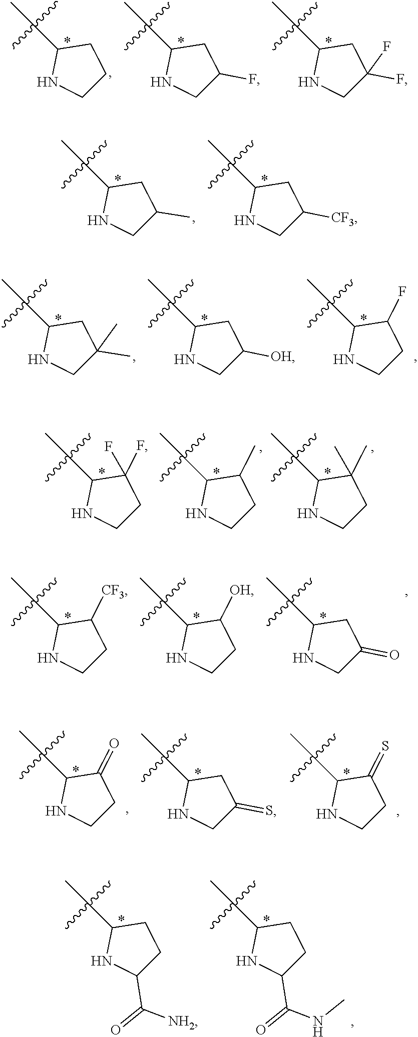

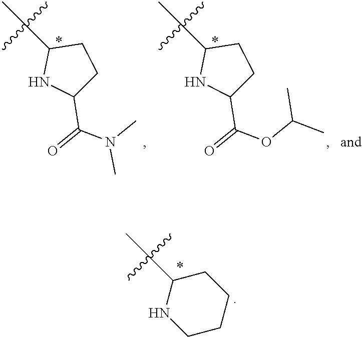 [SMILES: *C1CCC(C(=O)N(C)C)N1 ; *C1CCC(C(=O)OC(C)C)N1 ; *C1CCCCN1]

In some embodiments, the carbon atom labeled by the “*” sign is in a S configuration. In some embodiments, the carbon atom labeled by the “*” sign is in a R configuration.

In some embodiments, Y is NR³, and the

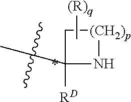

moiety in Formula IIC and its sub-formulas is selected from the following:

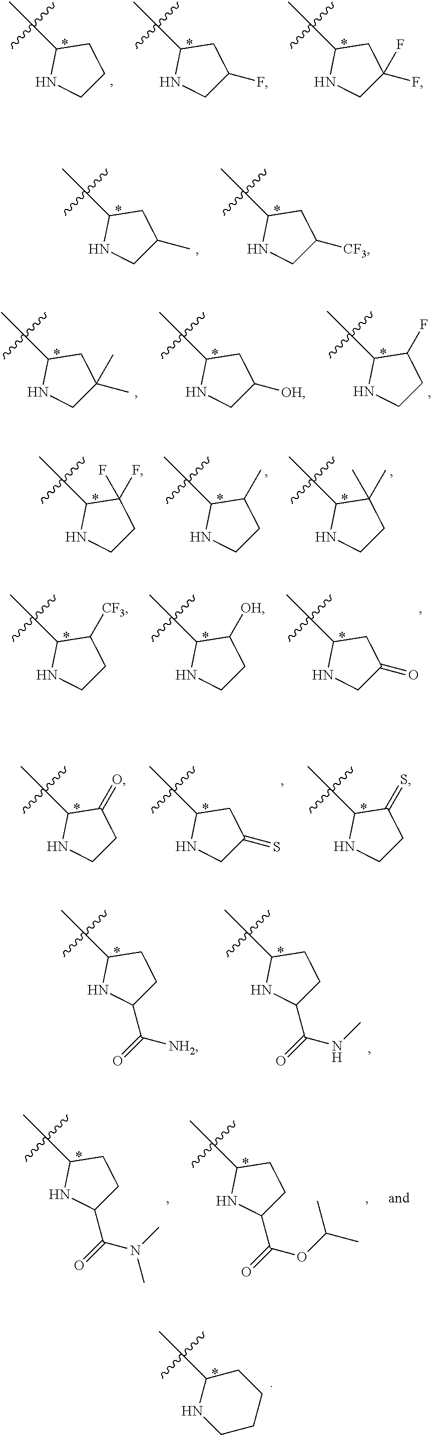

In some embodiments, the carbon atom labeled by the “*” sign is in a S configuration. In some embodiments, the carbon atom labeled by the “*” sign is in a R configuration. In some embodiments, R^{A} and R^{B} are hydrogen. In some embodiments, R^{A} is methyl, and R^{B} is hydrogen. In some embodiments, R^{A} and R^{B} are methyl. In some embodiments, R^{D} is hydrogen. In some embodiments, R^{D} is methyl. In some embodiments, R^{A}, R^{B}, and R^{D} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

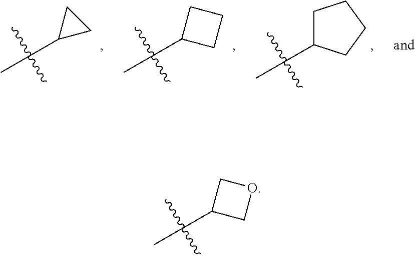

In some embodiments, R³ is

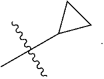 [SMILES: *C1CC1]

In some embodiments, R³ is

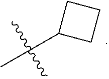 [SMILES: *C1CCC1]

In some embodiments, R³ is

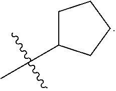 [SMILES: *C1CCCC1]

In some embodiments, R³ is

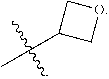 [SMILES: *C1COC1]

In some embodiments, the

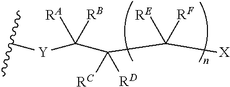

moiety in the prodrugs of Group II is selected from

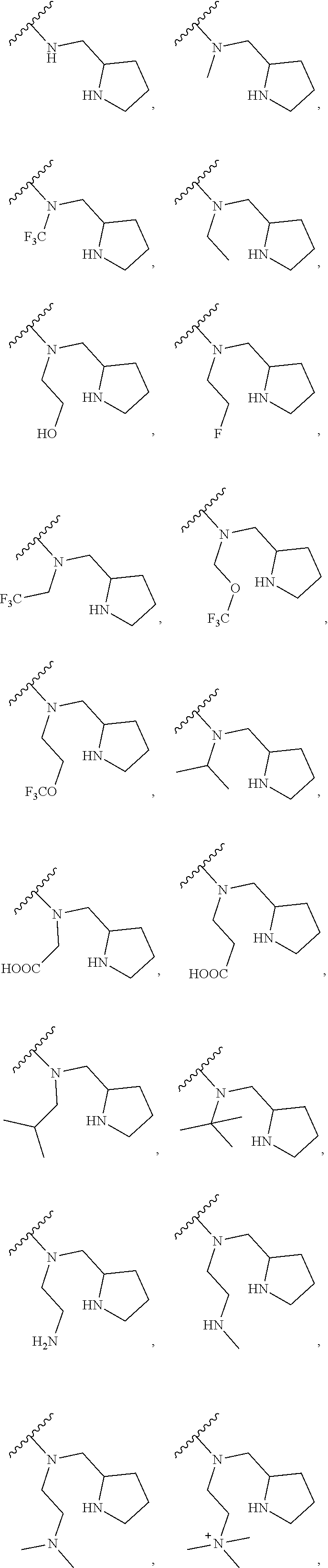

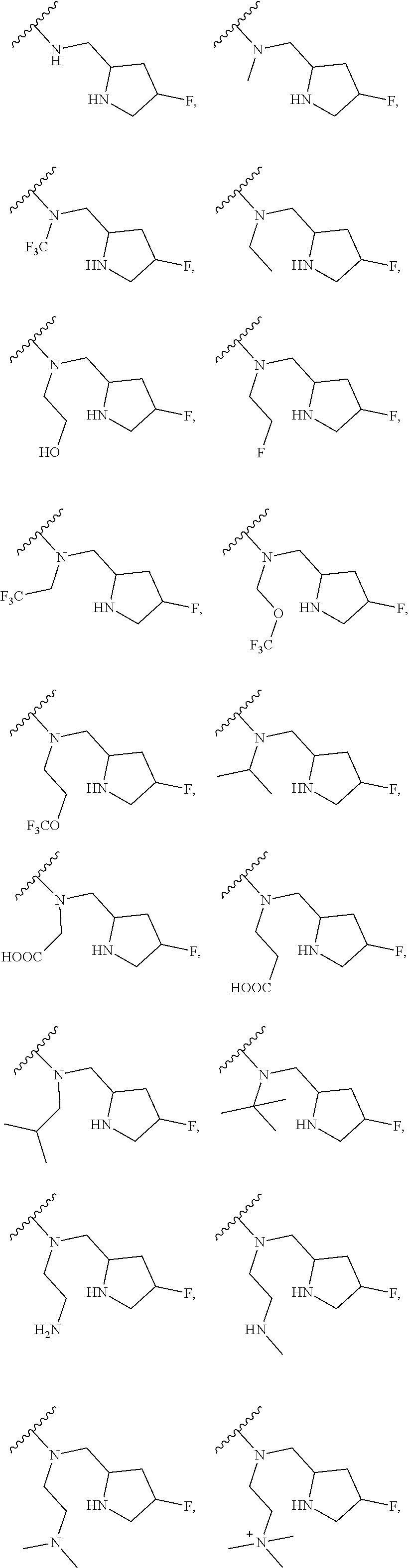

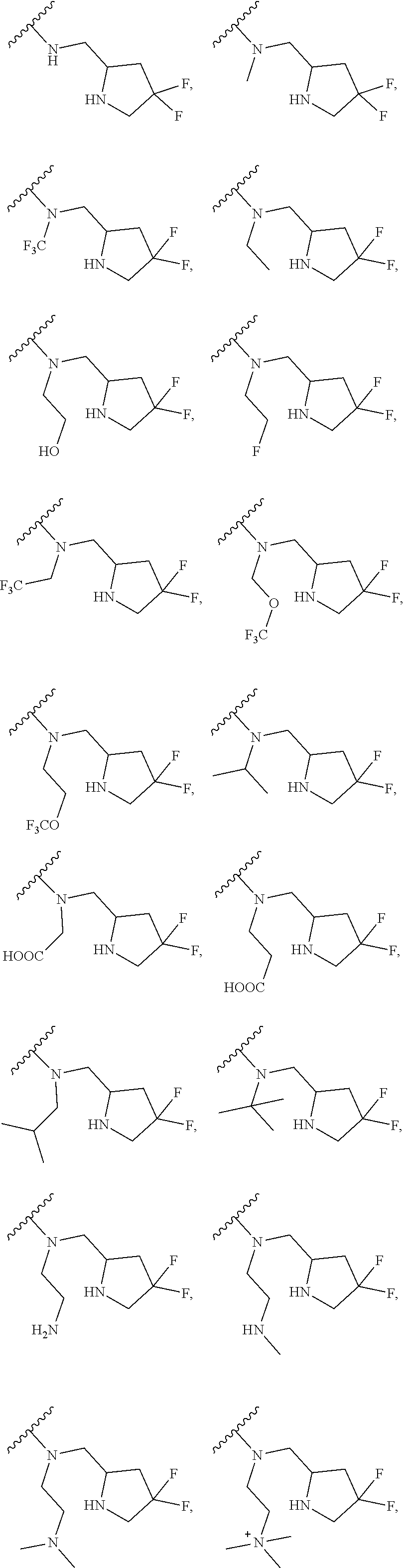

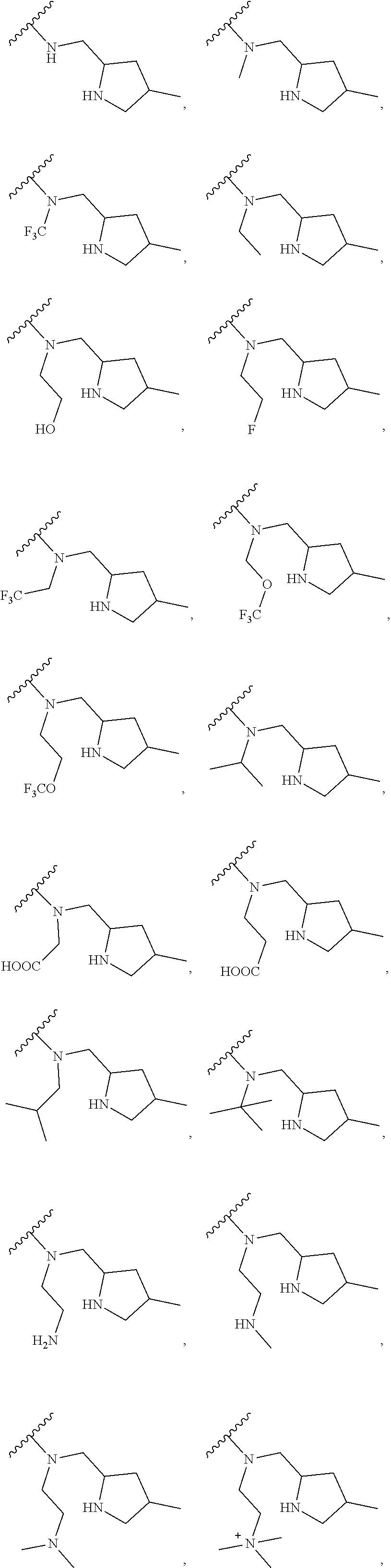

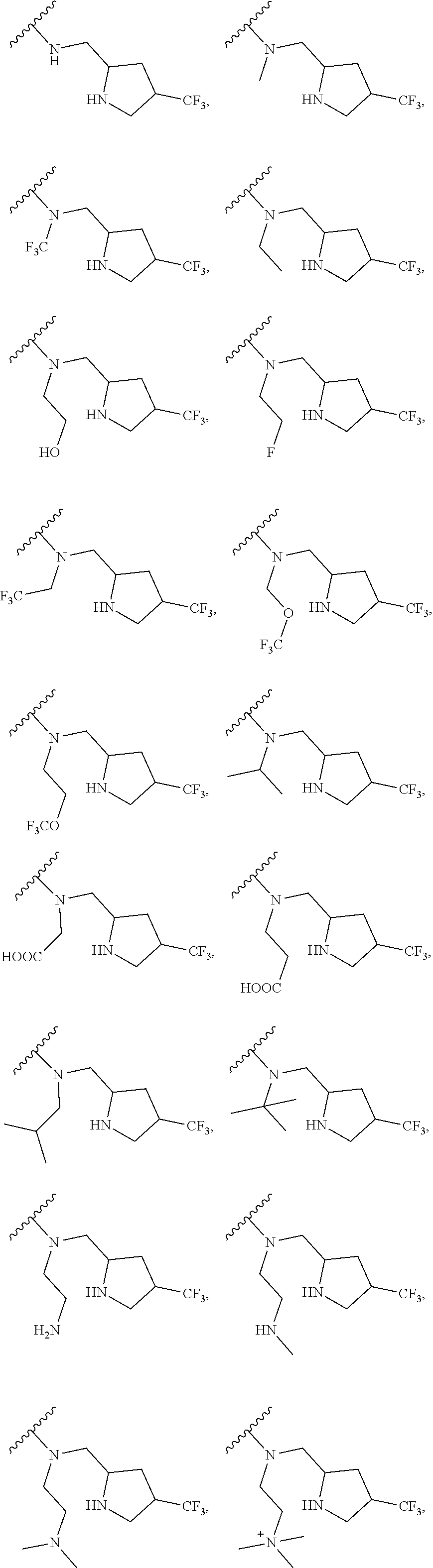

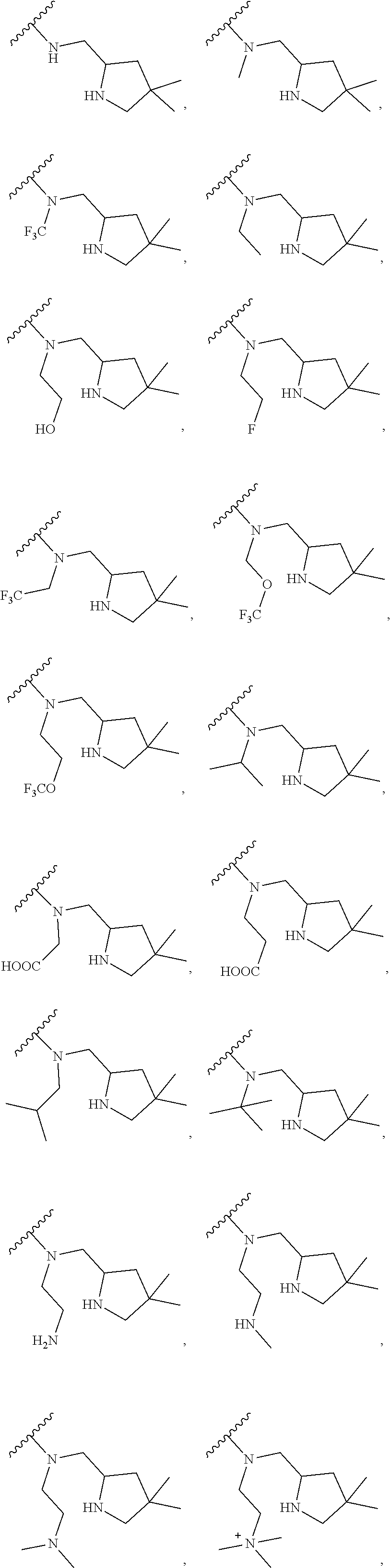

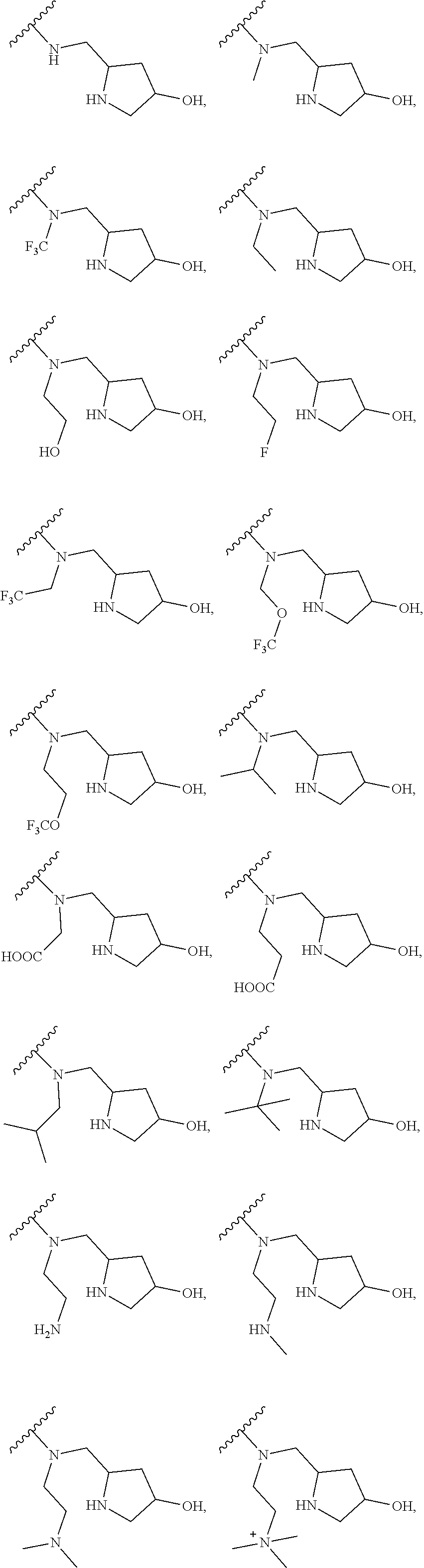

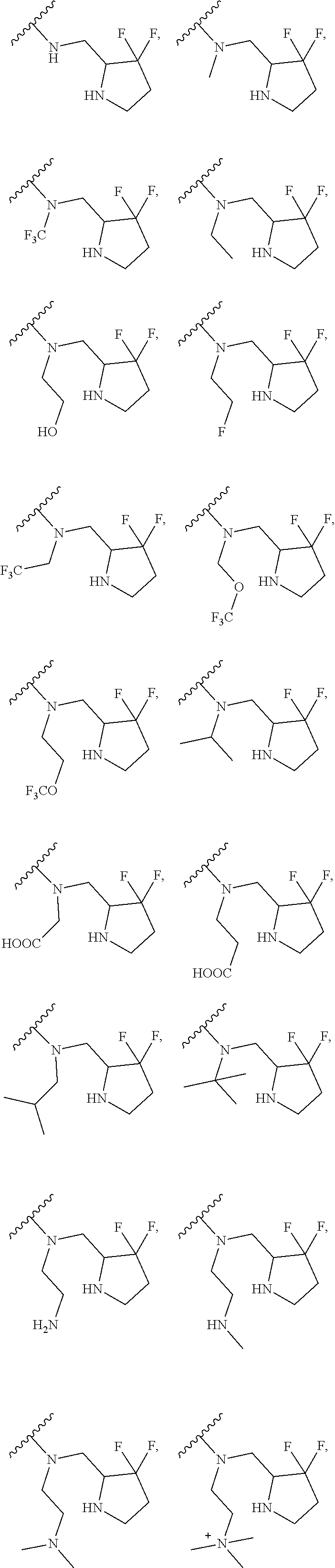

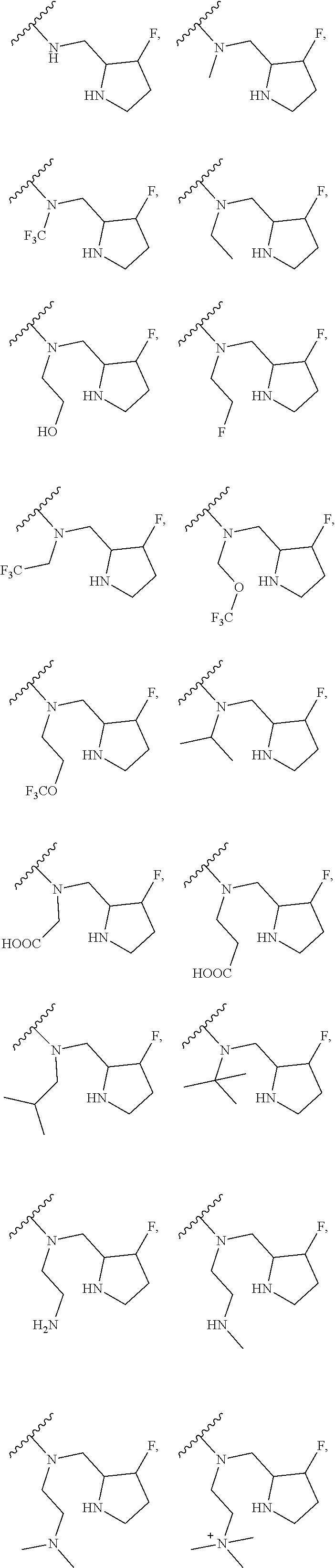

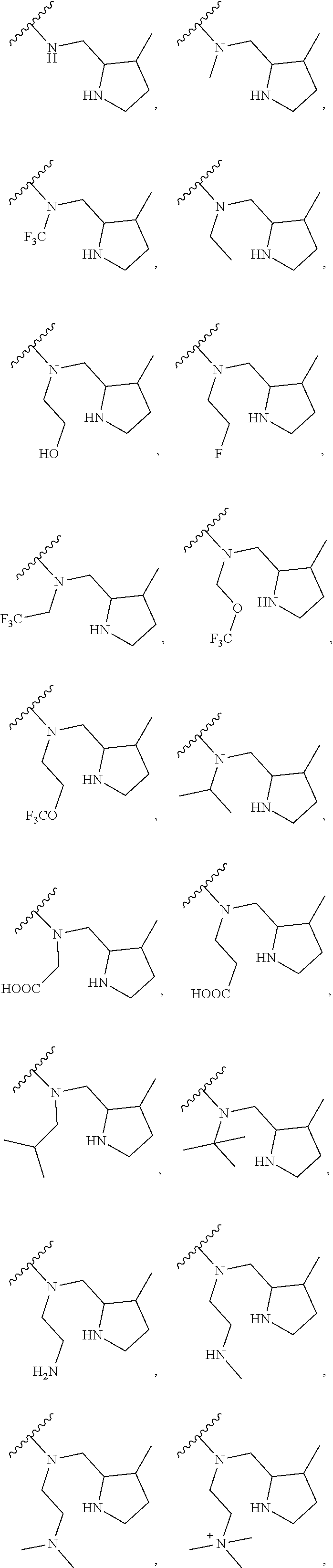

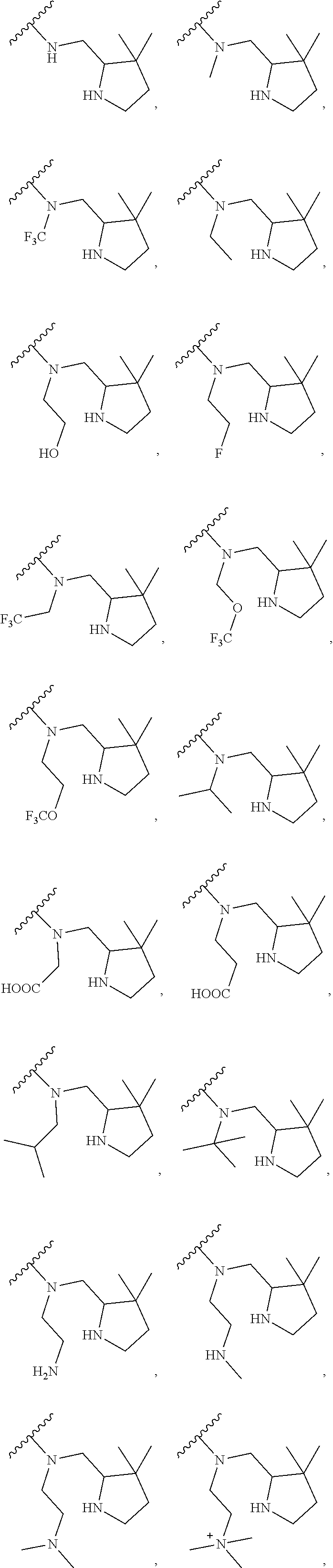

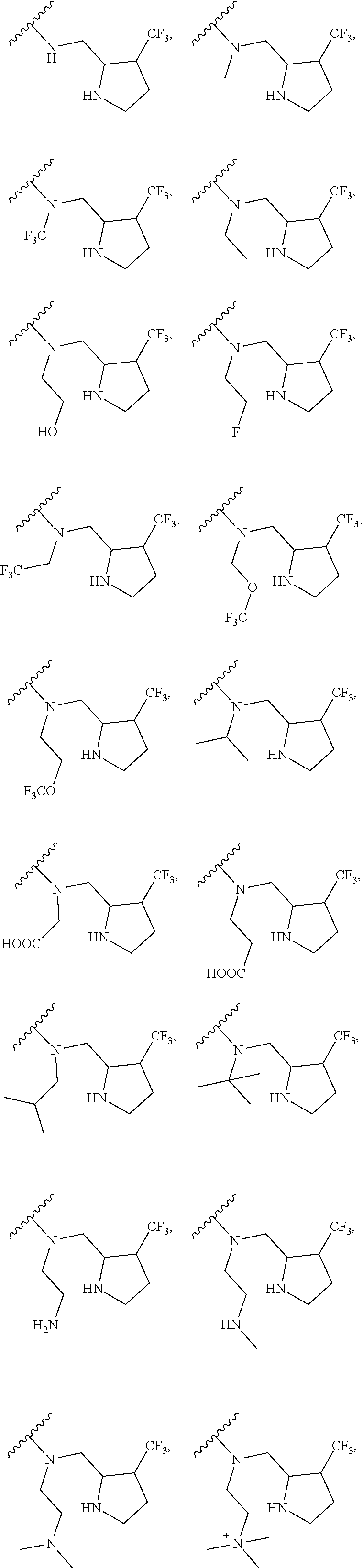

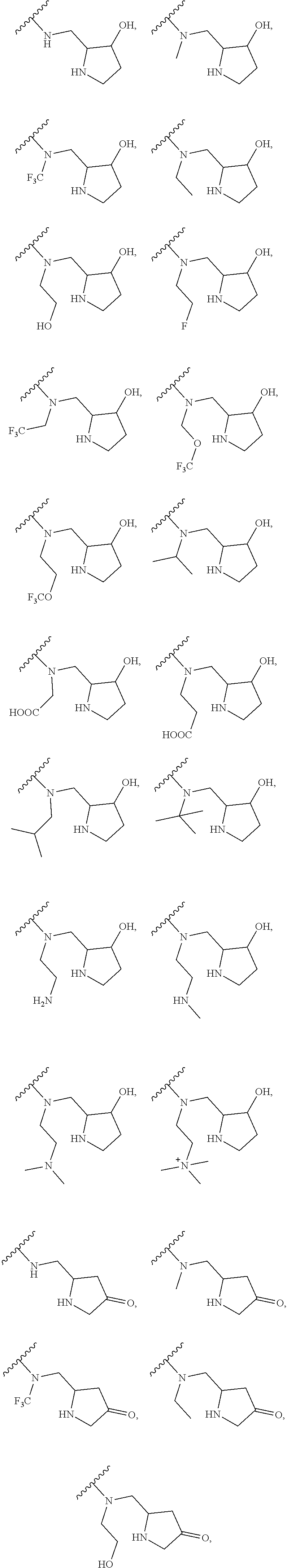

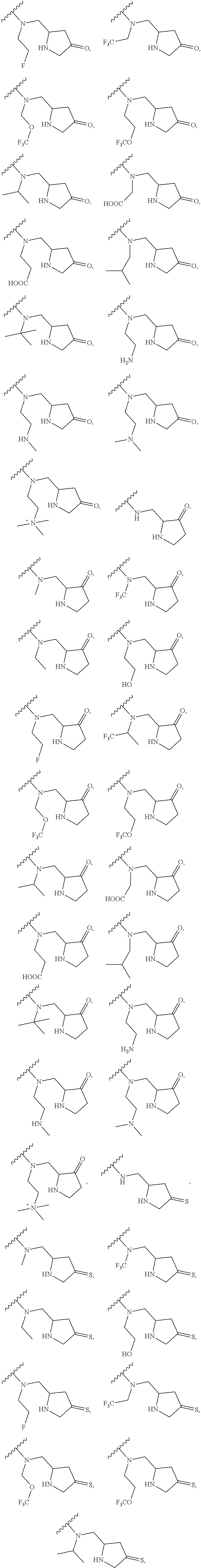

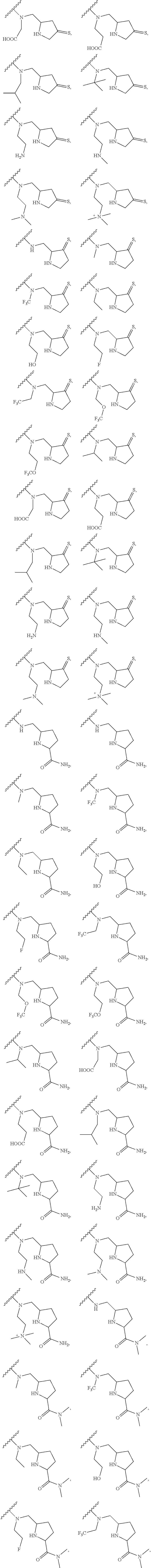

[chemical structure]

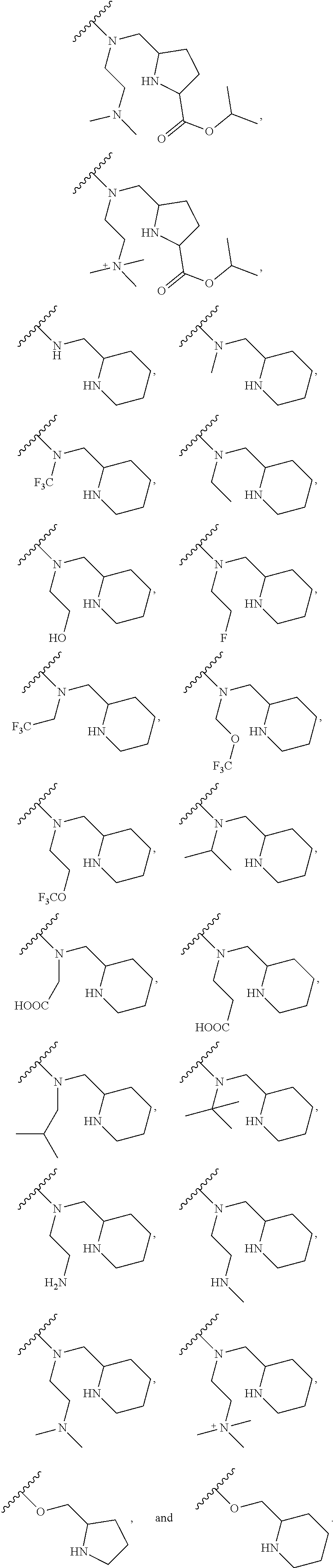

In some embodiments, the ring carbon atom in the pyrrolidine or piperidine moiety that connects to the rest of

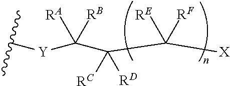

is in the S configuration. In some embodiments, the ring carbon atom in the pyrrolidine or piperidine moiety that connects to the rest of

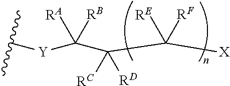

is in the R configuration.

In some embodiments, the

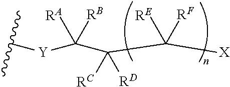

moiety in the prodrugs of Group II is selected from:

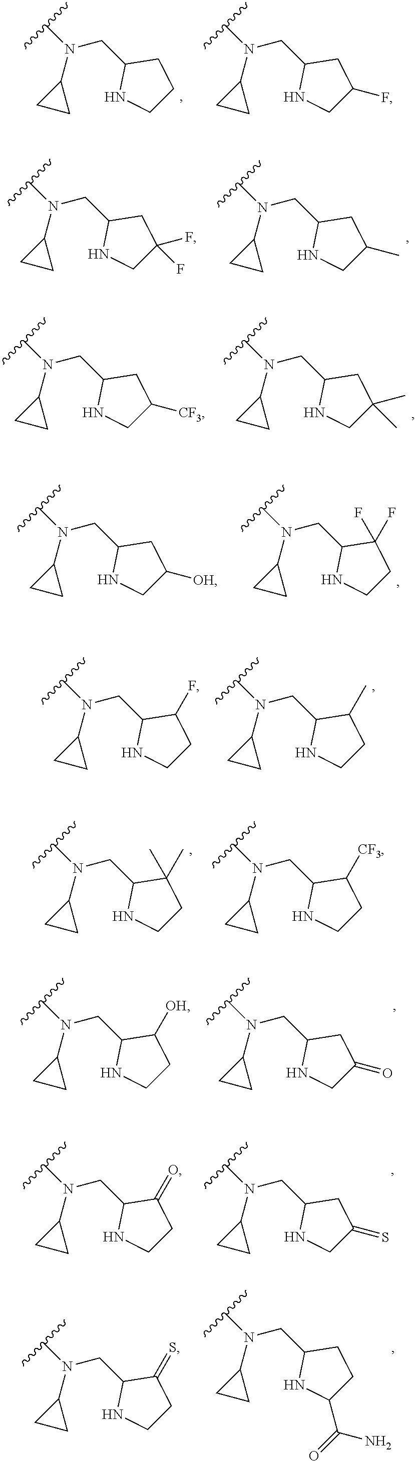

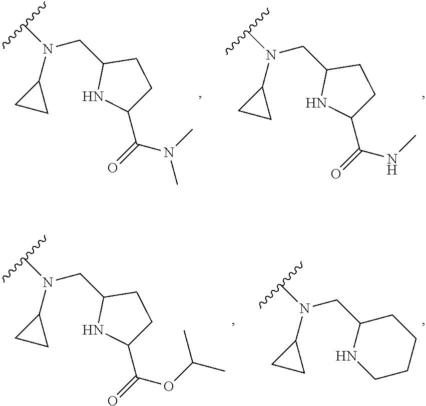 [SMILES: *N(CC1CCC(C(=O)N(C)C)N1)C1CC1 ; *N(CC1CCC(C(=O)NC)N1)C1CC1 ; *N(CC1CCC(C(=O)OC(C)C)N1)C1CC1 ; *N(CC1CCCCN1)C1CC1]

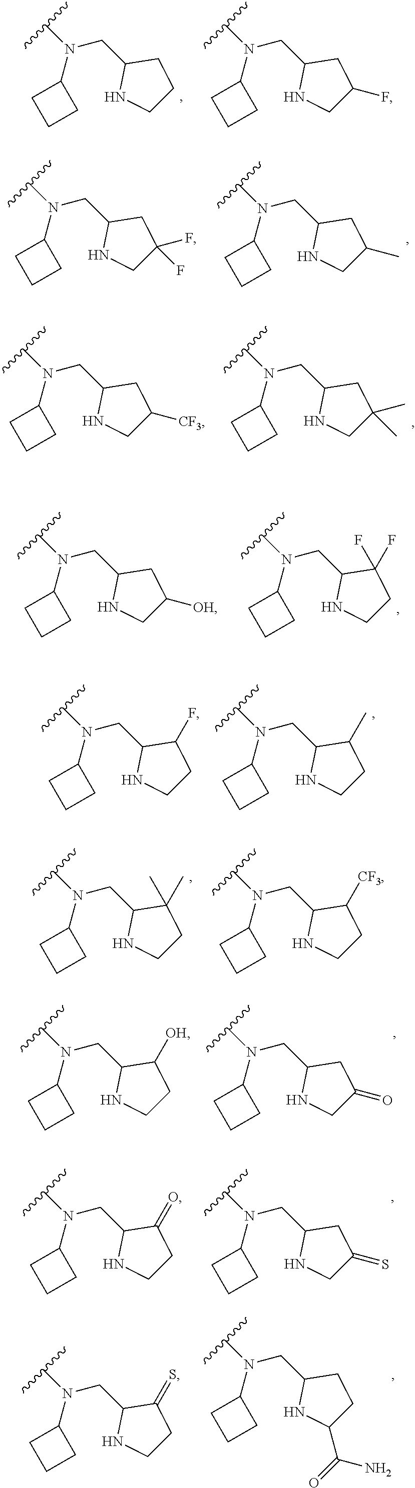

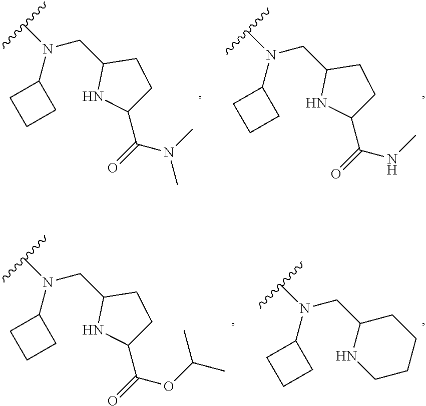

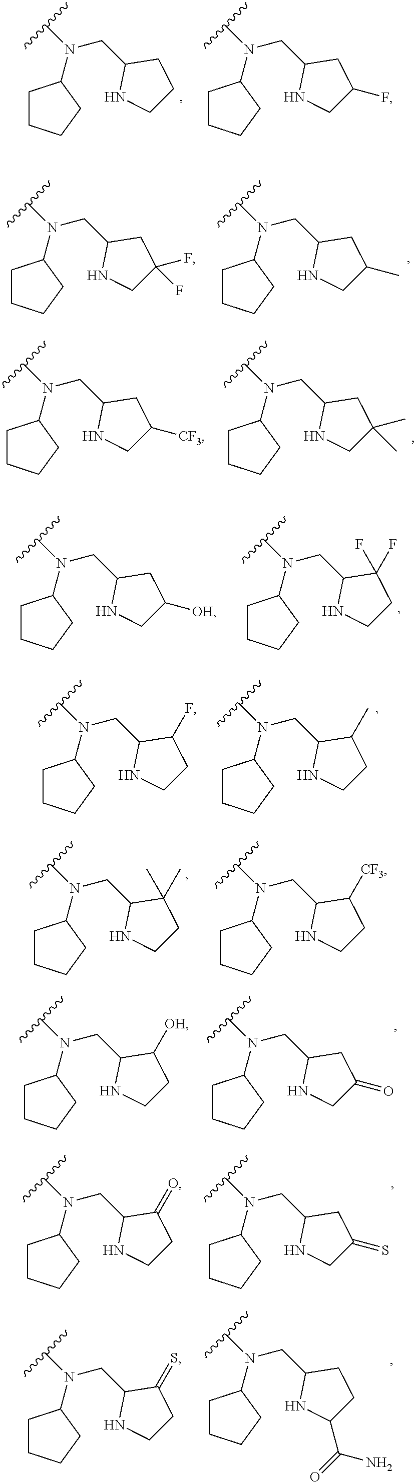

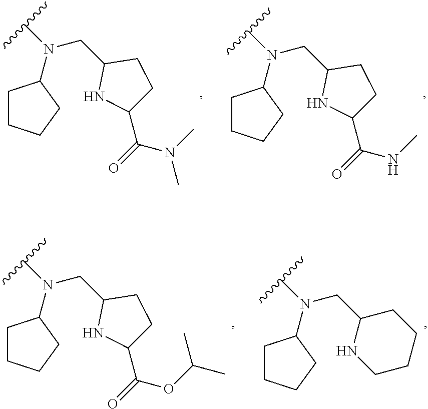

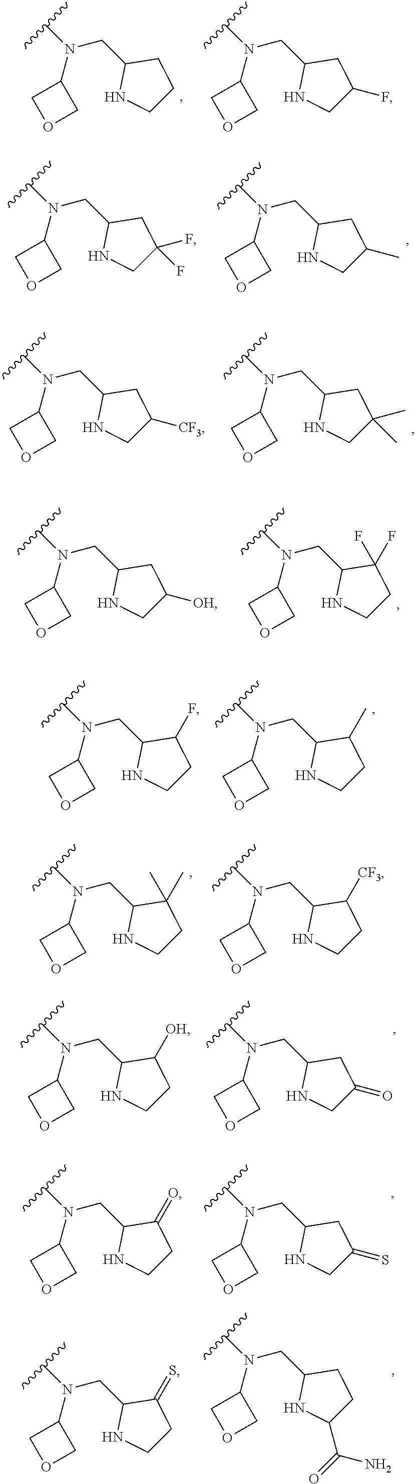

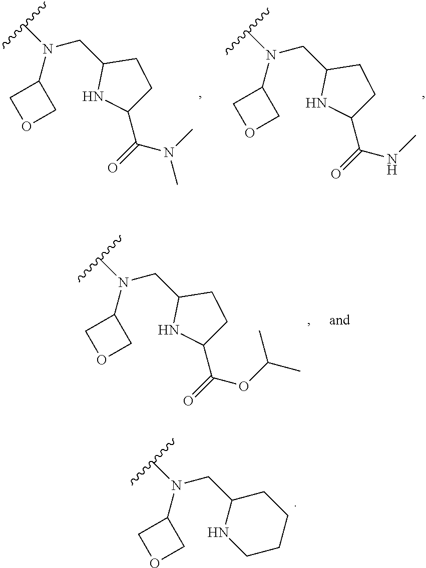 [SMILES: *N(CC1CCC(C(=O)N(C)C)N1)C1COC1 ; *N(CC1CCC(C(=O)NC)N1)C1COC1 ; *N(CC1CCC(C(=O)OC(C)C)N1)C1COC1 ; *N(CC1CCCCN1)C1COC1]

In some embodiments, the ring carbon atom in the pyrrolidine or piperidine moiety that connects to the rest of

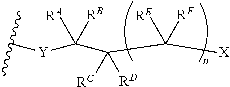

is in the S configuration. In some embodiments, the ring carbon atom in the pyrrolidine or piperidine moiety that connects to the rest of

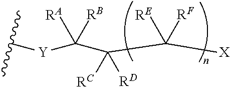

is in the R configuration.

In some embodiments, the

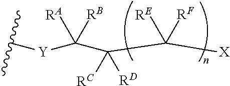

moiety in the prodrugs of Group II is selected from:

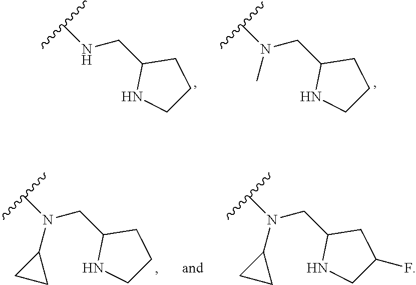

In some embodiments, the ring carbon atom in the pyrrolidine moiety that connects to the rest of

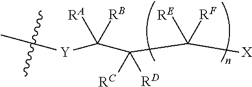

is in the S configuration. In some embodiments, the ring carbon atom in the pyrrolidine moiety that connects to the rest of

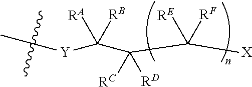

is in the R configuration.

In some embodiments, the

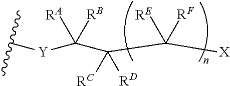

moiety in the prodrugs of Group II is

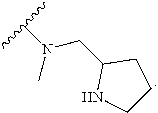 [SMILES: *N(C)CC1CCCN1]

In some embodiments, the

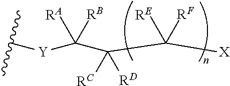

moiety in the prodrugs of Group II is

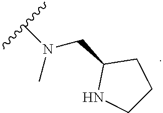 [SMILES: *N(C)C[C@H]1CCCN1]

In some embodiments, the

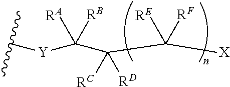

moiety in the prodrugs of Group II is

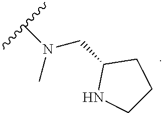 [SMILES: *N(C)C[C@@H]1CCCN1]

In some embodiments, Formula II is in the form of Formula IID,

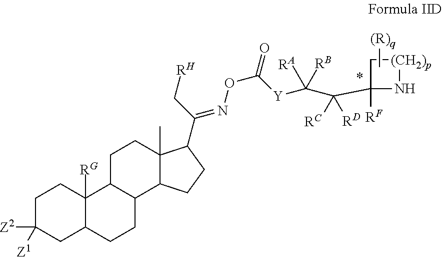

wherein Y, R^{A}, R^{B}, R^{C}, R^{D}, and R^{F} are the same as those described above in this section (i.e., Group II),
wherein p is an integer selected from 1-4,
wherein q is an integer selected from 0-6, and
wherein R is the same as those described above.

In some embodiments, the prodrugs are in a non-salt form as shown in Formula IID. In some embodiments, the prodrugs are in a salt form. In some embodiments, the prodrugs are in an HCl salt form.

In some embodiments, Formula IID is in the form of Formula IID-1. In some embodiments, the prodrugs are in a non-salt form as shown in Formula IID-1. In some embodiments, the prodrugs are in a salt form. In some embodiments, the prodrugs are in an HCl salt form.

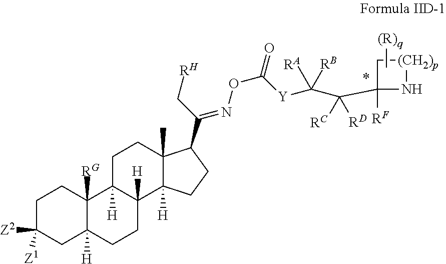

In some embodiments, Formula IID is in the form of Formula IID-2. In some embodiments, the prodrugs are in a non-salt form as shown in Formula IID-2. In some embodiments, the prodrugs are in a salt form. In some embodiments, the prodrugs are in an HCl salt form.

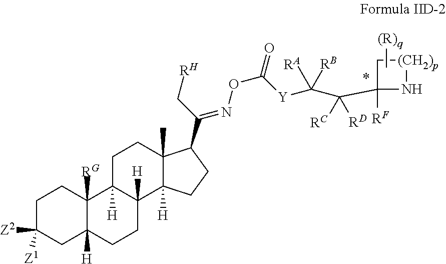

In some embodiments, the carbon atom labeled by the “*” sign is in a S configuration. In some embodiments, the carbon atom labeled by the “*” sign is in a R configuration.

In some embodiments, p is 1, 2, or 3. In some embodiments, p is 1. In some embodiments, p is 2. In some embodiments, p is 3.

In some embodiments, q is 0, 1, or 2. In some embodiments, q is 0. In some embodiments, q is 1. In some embodiments, q is 2.

In some embodiments, p is 1, 2, or 3, and q is 0, 1, or 2. In some embodiments, p is 2, and q is 0, 1, or 2. In some embodiments, p is 2, and q is 0. In some embodiments, p is 3, and q is 0, 1, or 2. In some embodiments, p is 3, and q is 0.

In some examples, the R groups are independently selected from halogen, nitro, cyano, hydroxyl, formyl, carboxyl, thiol, ═O (counting as two R groups), ═S (counting two R groups), sulfamoyl, alkyl (such as methyl, ethyl, isopropyl, tert-butyl), haloalkyl (such as trifluoromethyl), alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, arylalkyl (such as benzyl), alkylaryl, alkyloxy (such as methoxy, ethoxy), haloalkyloxy (such as trifluoromethoxy), aryloxy, alkylcarbonyl (such as acetyl), arylcarbonyl (such as benzoyl), alkylcarbonyloxy (such as acetoxy), arylcarbonyloxy (such as benzoyloxy), alkyloxycarbonyl (such as methoxycarbonyl, ethoxycarbonyl, isopropoxycarbonyl), aryloxycarbonyl, primary amino, alkylamino (such as methylamino, ethylamino, dimethylamino, diethylamino, N-methyl-N-ethylamino), alkylammonium (such as trimethylammonium), alkylcarbonylamino (such as acetylamino), arylcarbonylamino (such as benzoylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), and N-alkylsulfamoyl (such as N-methylsulfamoyl, N-ethylsulfamoyl, N,N-dimethylsulfamoyl, N,N-diethylsulfamoyl, N-methyl-N-ethylsulfamoyl).

In some examples, the R groups are independently selected from halogen, nitro, cyano, hydroxyl, trifluoromethyl, methoxy, ethoxy, trifluoromethoxy, primary amino, formyl, carboxyl, carbamoyl, thiol, ═O, ═S, sulfamoyl, acetyl, acetoxy, methoxycarbonyl, ethoxycarbonyl, isopropoxycarbonyl, methylamino, ethylamino, dimethylamino, diethylamino, N-methyl-N-ethylamino, trimethylammonium, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, N-methylsulfamoyl, N-ethylsulfamoyl, NN-dimethylsulfamoyl, N,N-diethylsulfamoyl, N-methyl-N-ethylsulfamoyl, benzyl, benzoyl, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, and haloheteroaryl.

In some examples, the R groups are independently selected from halogen, ═O, ═S, alkyl, haloalkyl, carbocyclyl, halocarbocyclyl, aryl, haloaryl, heterocyclyl, and haloheterocyclyl.

In some examples, the R groups are independently selected from halogen, ═O, ═S, alkyl, haloalkyl, carbocyclyl, halocarbocyclyl, aryl, haloaryl, heterocyclyl, haloheterocyclyl, carbamoyl, N-alkylcarbamoyl, alkyloxycarbonyl, and aryloxycarbonyl.

In some examples, q is an integer selected from 1-6, and one of the R groups is carbamoyl or N-alkylcarbamoyl. For example, q is 1 and R is carbamoyl.

In some examples, q is an integer selected from 1-6, and one of the R groups is alkyloxycarbonyl or aryloxycarbonyl. For example, q is 1 and R is isopropoxycarbonyl.

In some examples, q is an integer selected from 1-6, and one of the R groups is fluorine. For example, q is 2 and both R groups are fluorine (they can be attached to the same atom or two different atoms).

In some examples, q is an integer selected from 2-6, and two of the R groups constitute ═O or ═S. For example, q is 2 and the two R groups constitute ═O.

In some embodiments, the

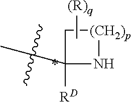

moiety in Formula IID and its sub-formulas is selected from the following:

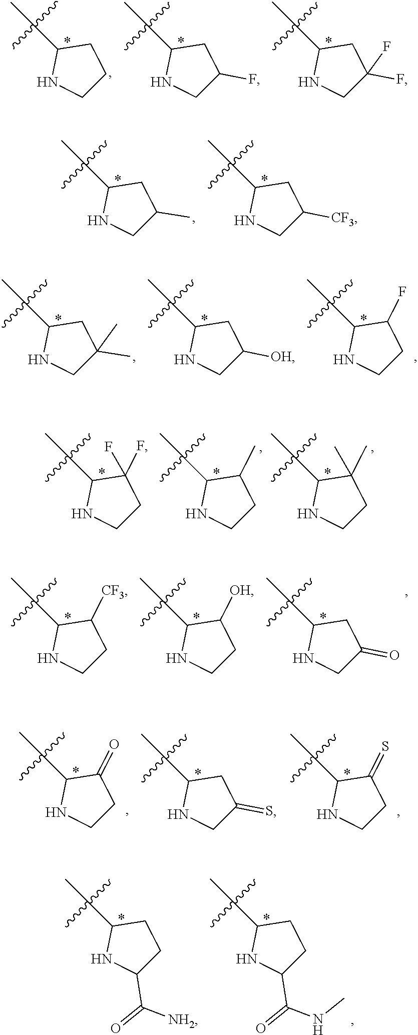

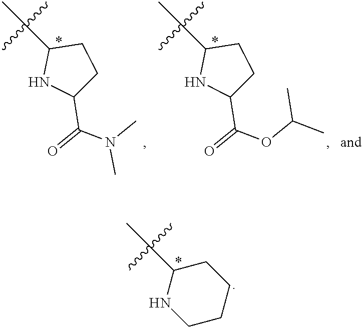 [SMILES: *C1CCC(C(=O)N(C)C)N1 ; *C1CCC(C(=O)OC(C)C)N1 ; *C1CCCCN1]

In some embodiments, the carbon atom labeled by the “*” sign is in a S configuration. In some embodiments, the carbon atom labeled by the “*” sign is in a R configuration.

In some embodiments, Y is NR³, and the

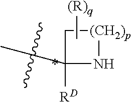

moiety in Formula IID and its sub-formulas is selected from the following:

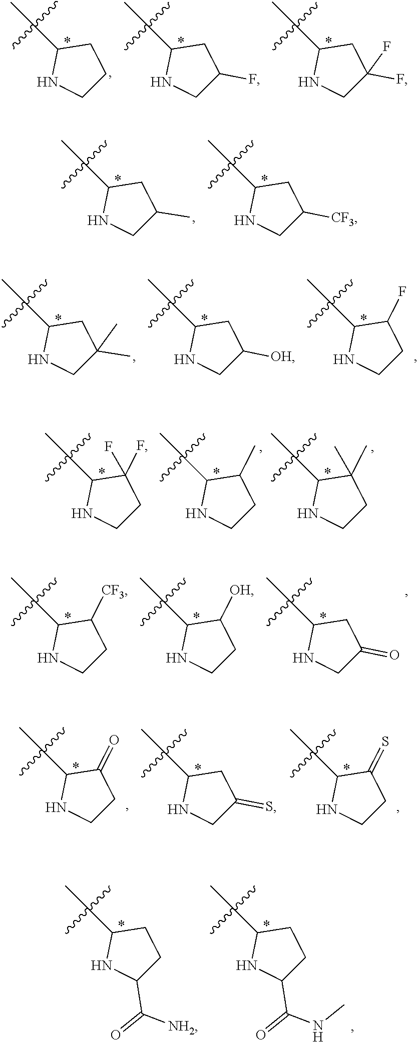

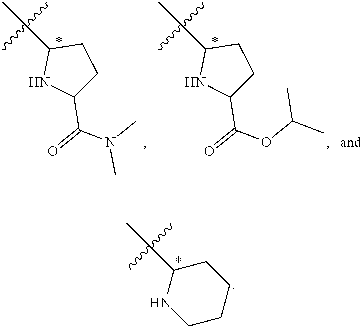 [SMILES: *C1CCC(C(=O)N(C)C)N1 ; *C1CCC(C(=O)OC(C)C)N1 ; *C1CCCCN1]

In some embodiments, the carbon atom labeled by the “*” sign is in a S configuration. In some embodiments, the carbon atom labeled by the “*” sign is in a R configuration. In some embodiments, R^{A} and R^{B} are hydrogen. In some embodiments, R^{A} is methyl, and R^{B} is hydrogen. In some embodiments, R^{A} and R^{B} are methyl. In some embodiments, R^{C} and R^{D} are hydrogen. In some embodiments, R^{C} is methyl, and R^{D} is hydrogen. In some embodiments, and R^{C} and R^{D} are methyl. In some embodiments, R^{F} is hydrogen. In some embodiments, R^{F} is methyl. In some embodiments, R^{A}, R^{B}, R^{C}, R^{D}, and R^{F} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

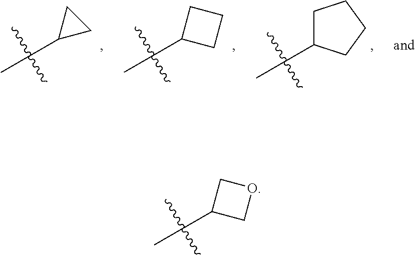

In some embodiments, R³ is

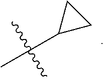 [SMILES: *C1CC1]

In some embodiments, R³ is

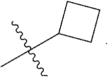 [SMILES: *C1CCC1]

In some embodiments, R³ is

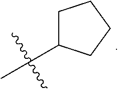 [SMILES: *C1CCCC1]

In some embodiments, R³ is

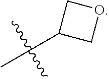 [SMILES: *C1COC1]

In some embodiments, the

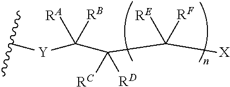

moiety in the prodrugs of Group II is selected from:

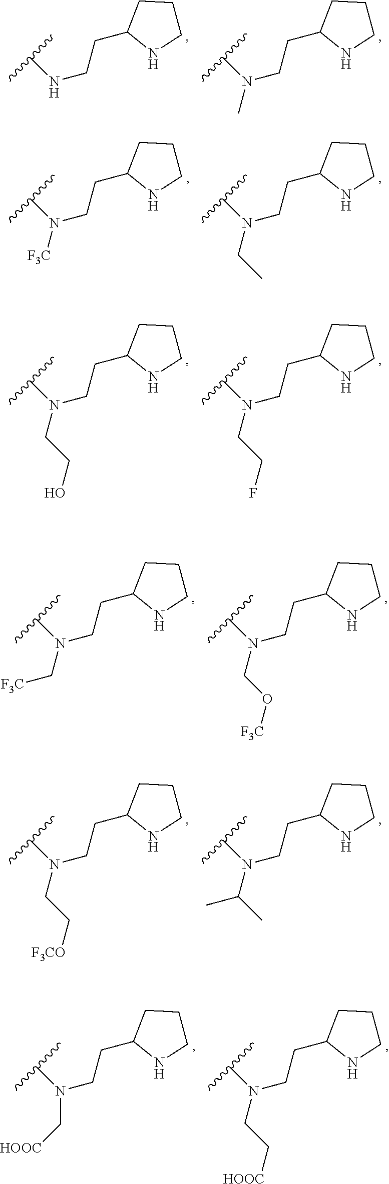

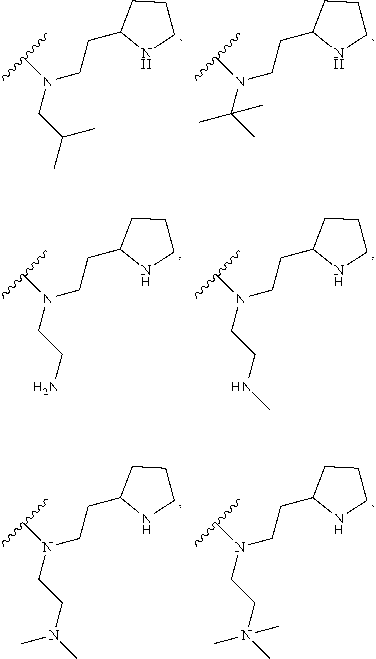

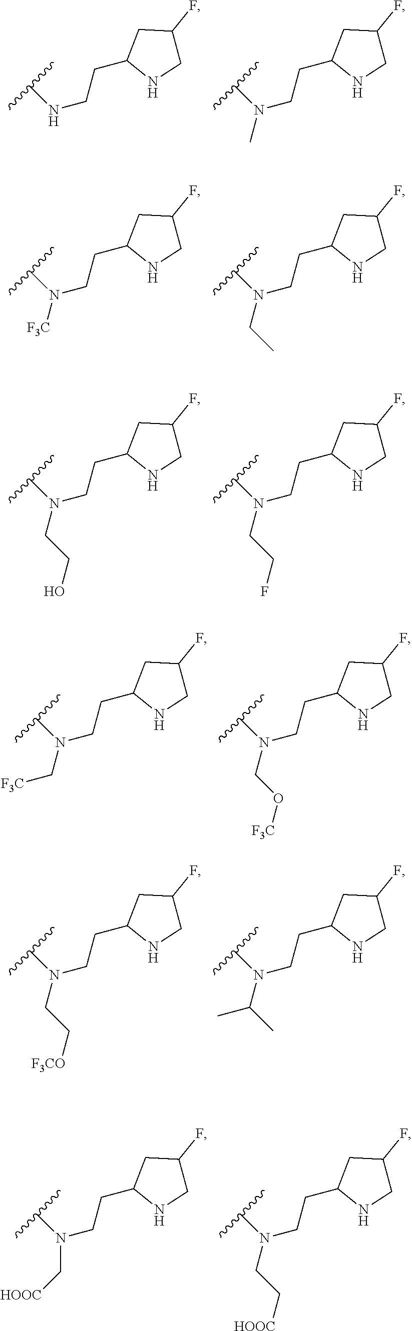

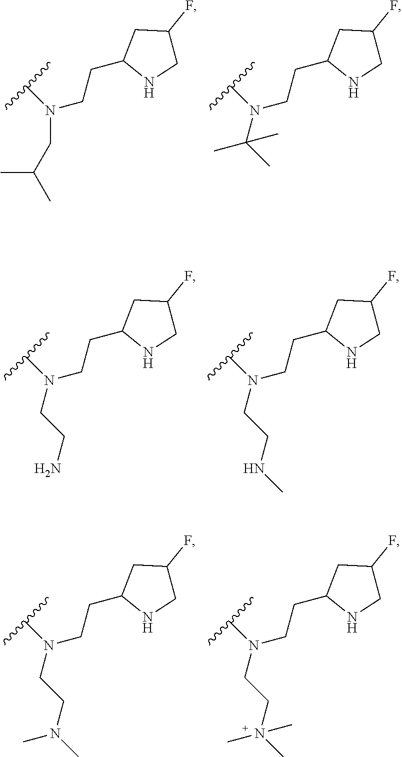

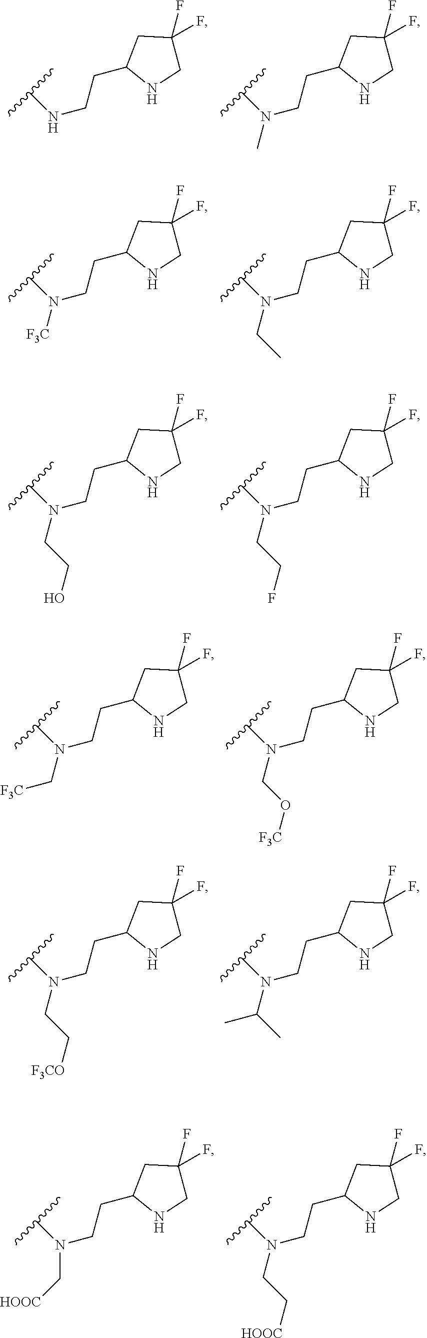

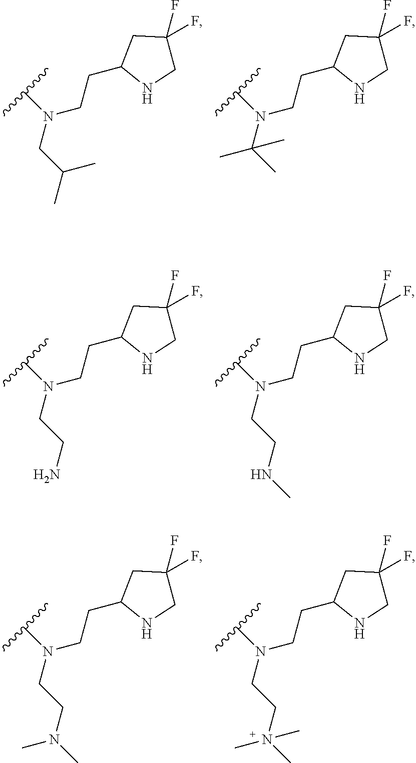

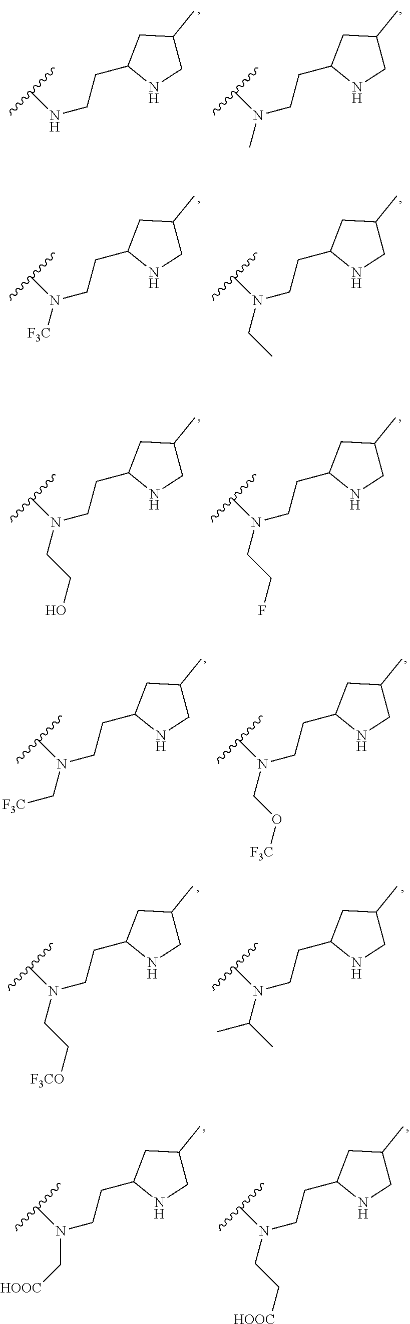

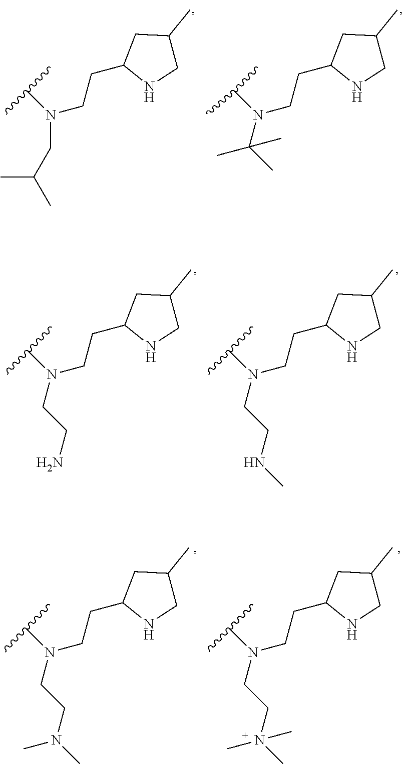

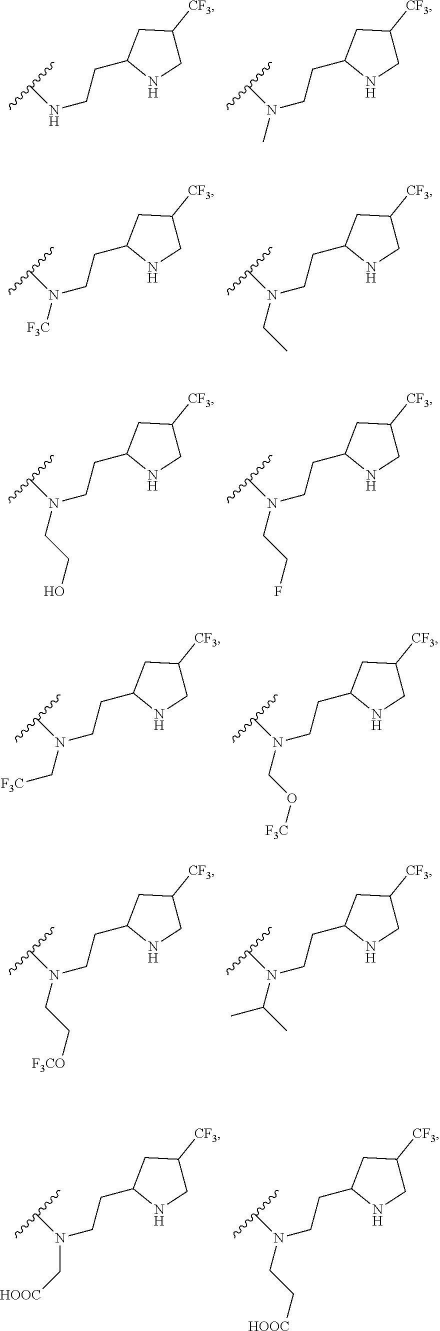

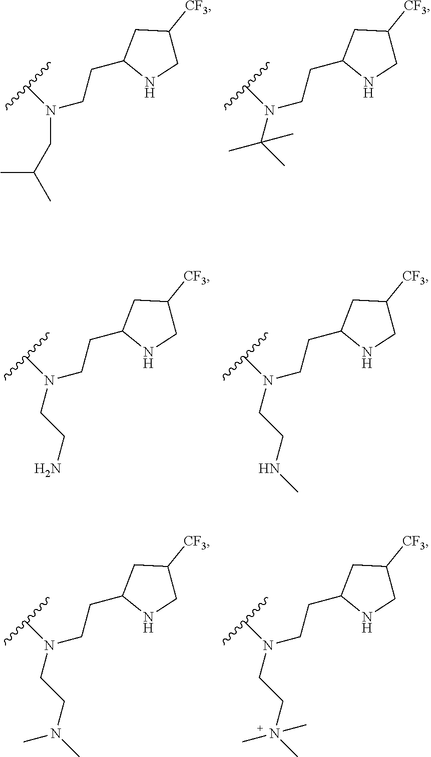

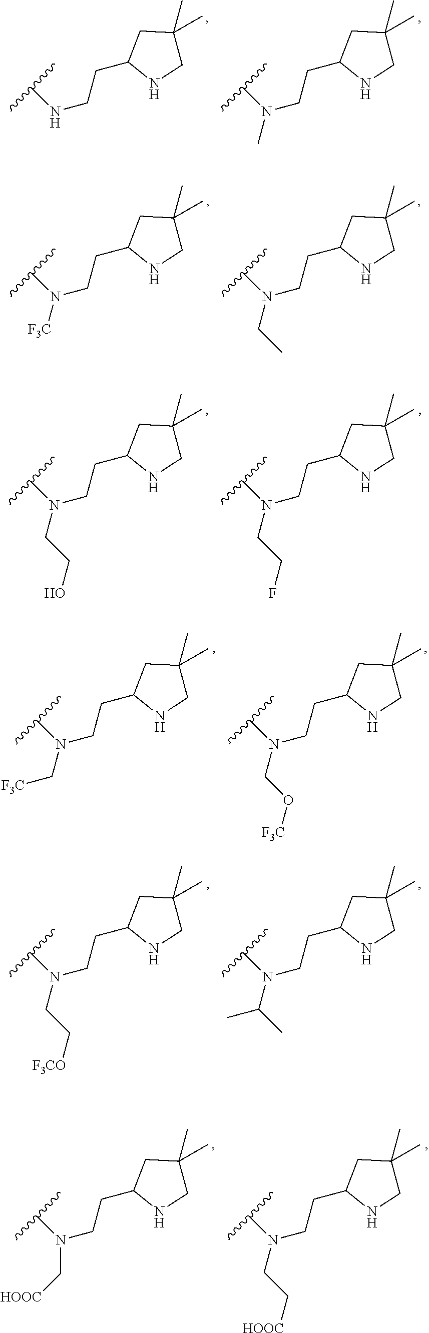

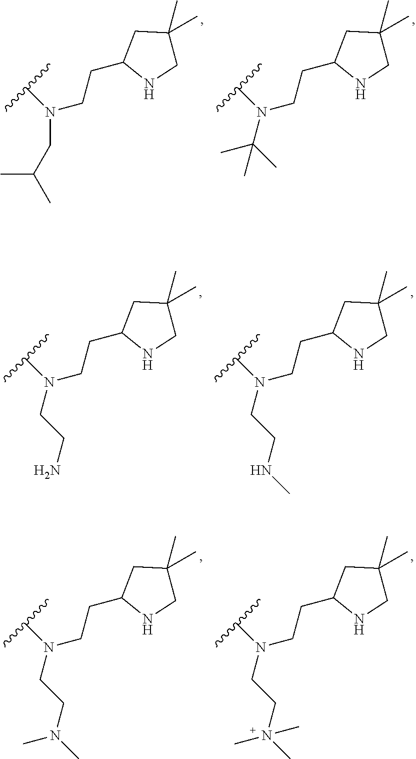

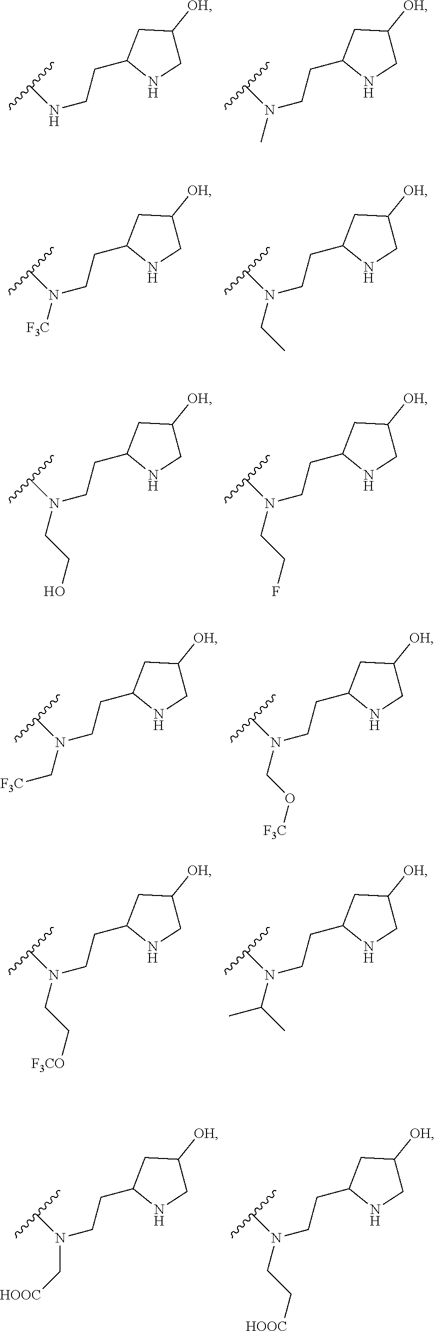

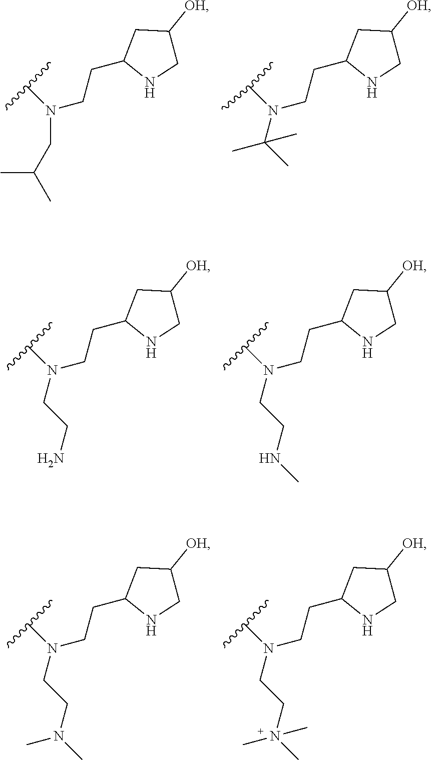

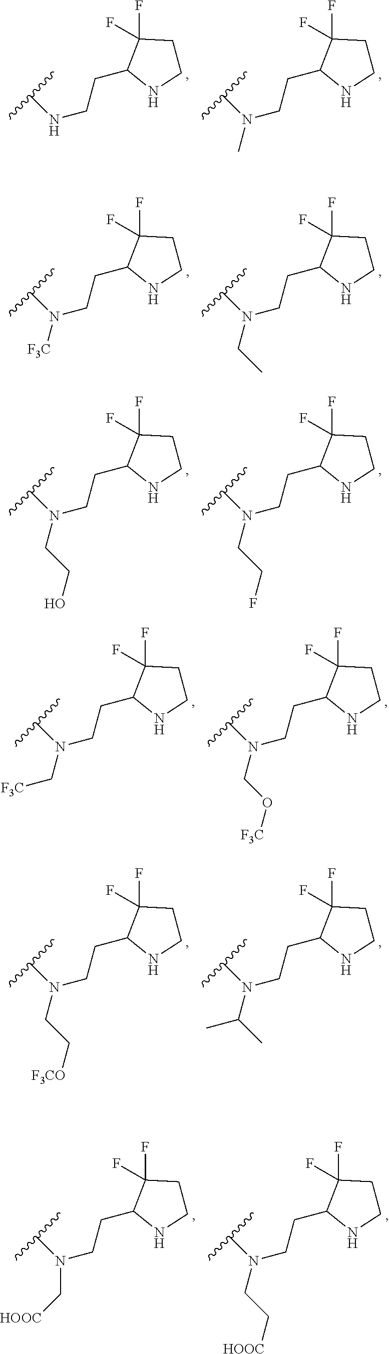

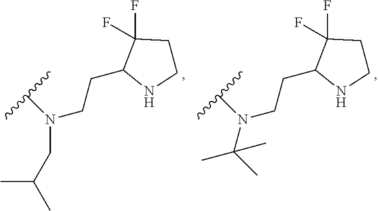 [SMILES: *N(CCC1NCCC1(F)F)CC(C)C ; *N(CCC1NCCC1(F)F)C(C)(C)C]

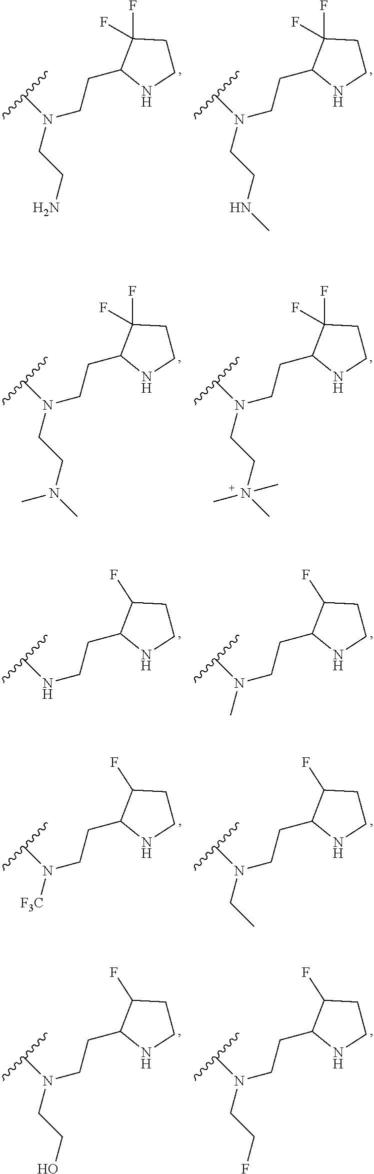

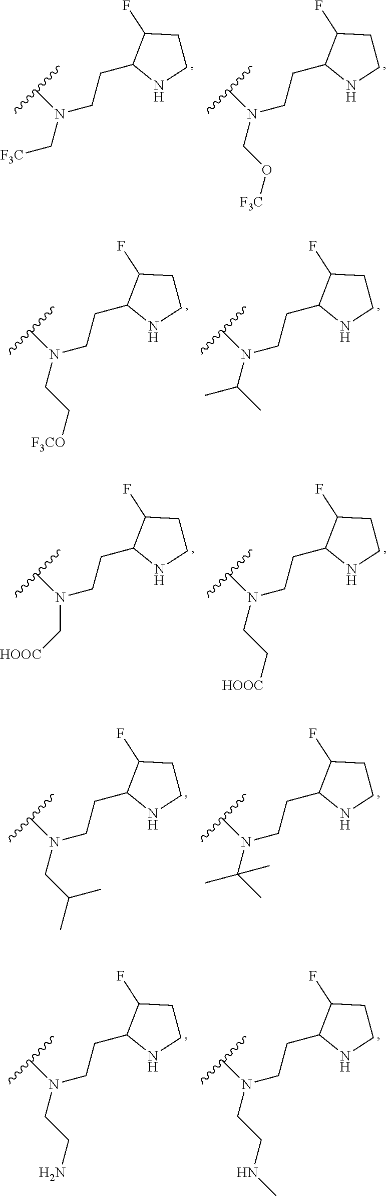

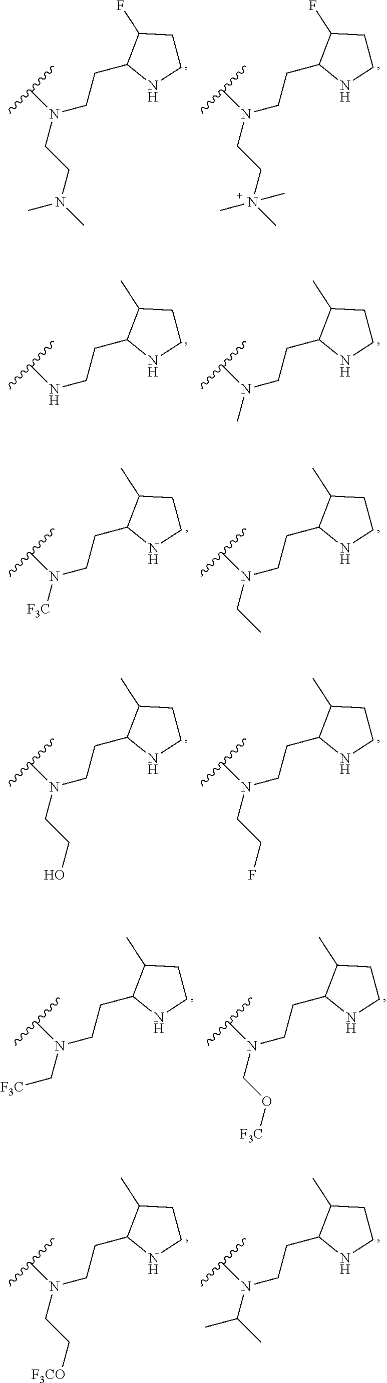

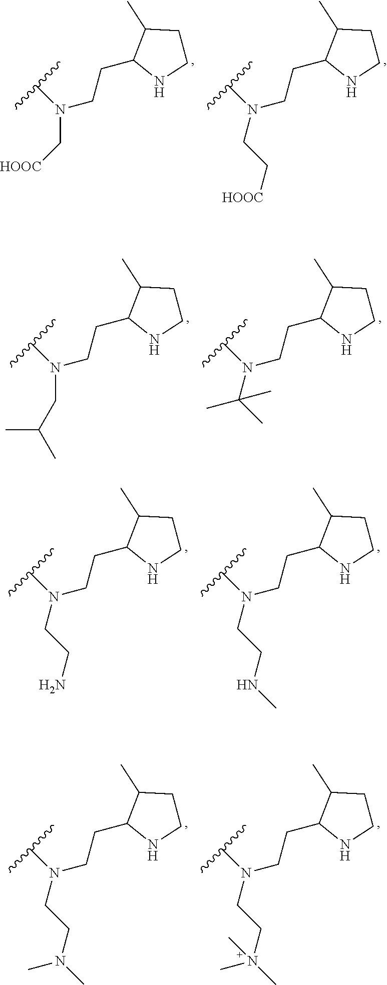

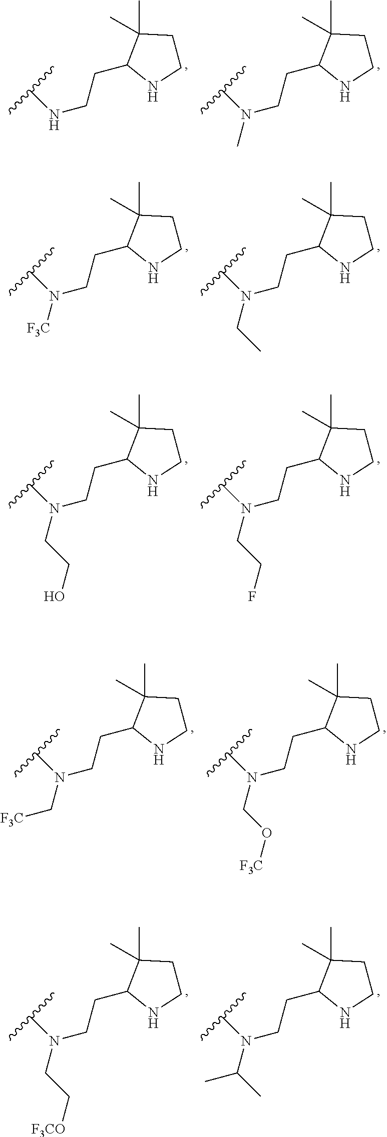

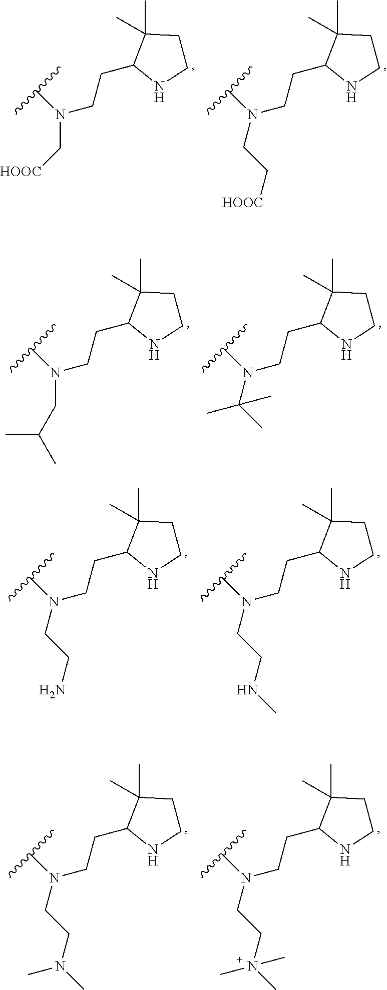

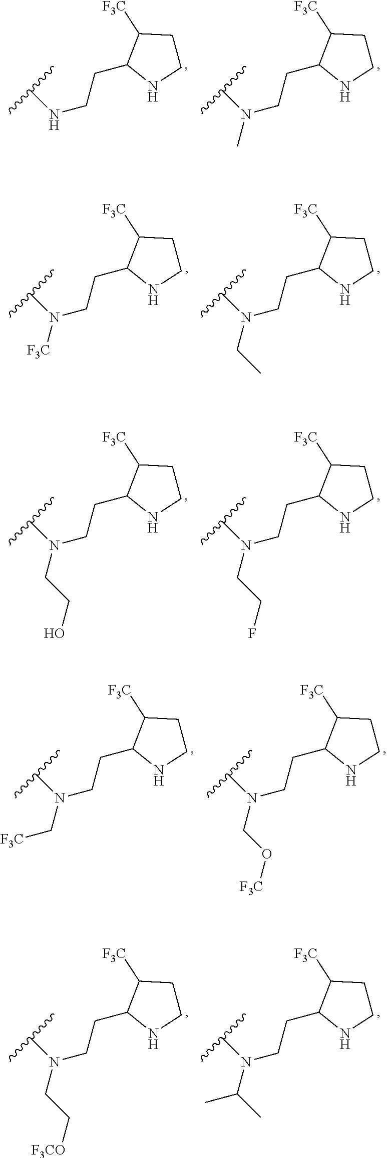

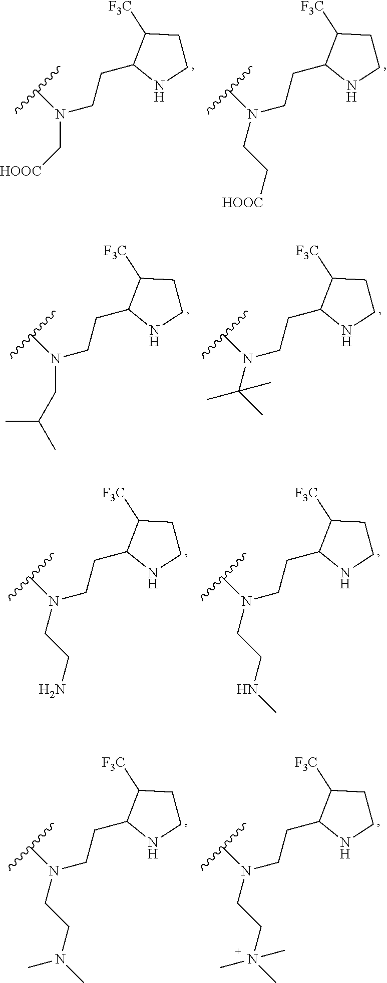

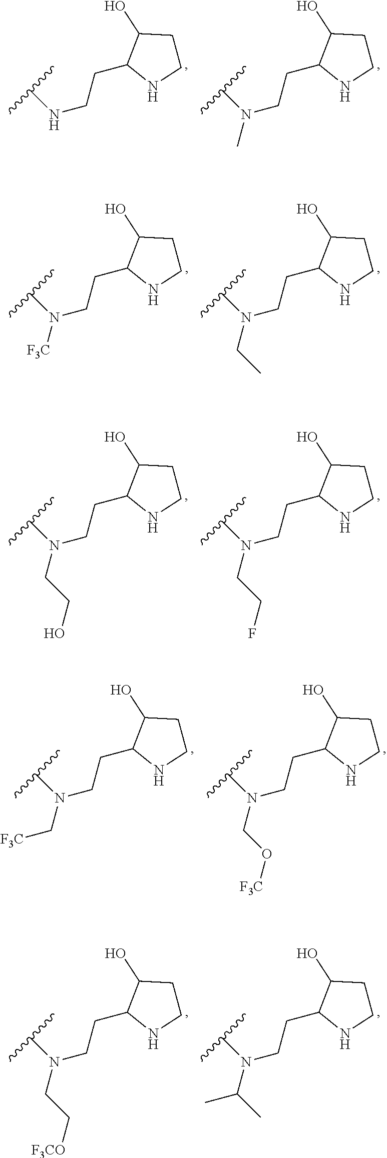

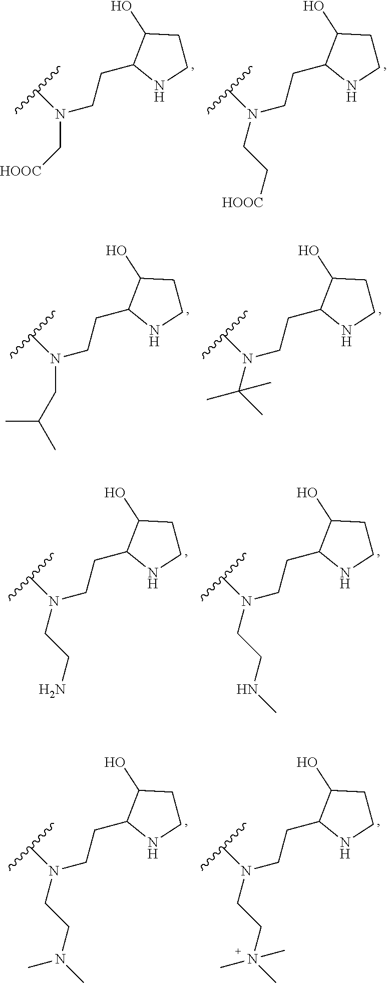

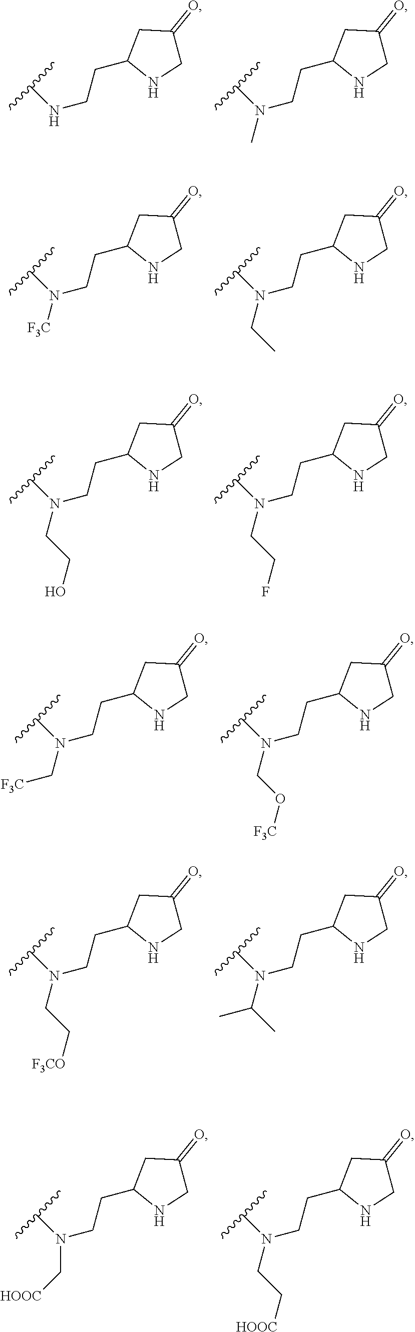

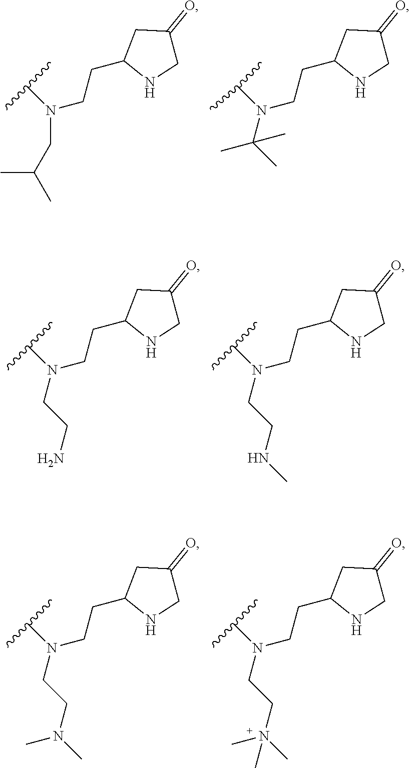

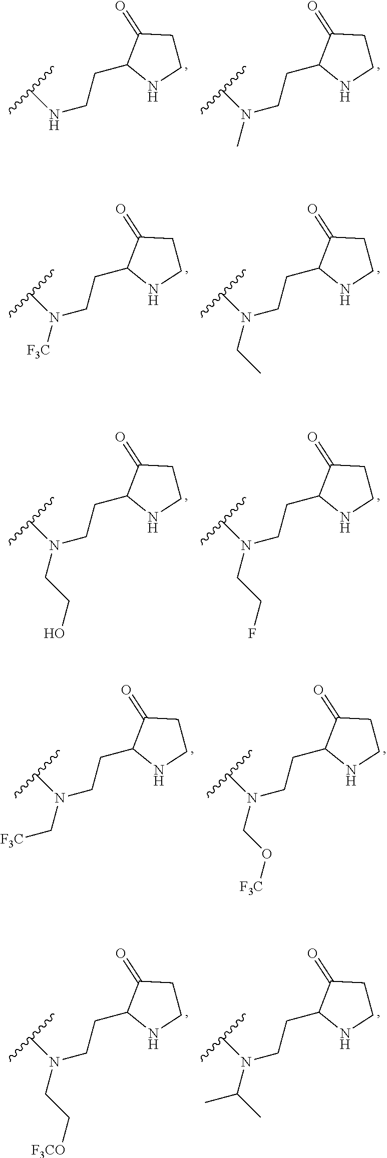

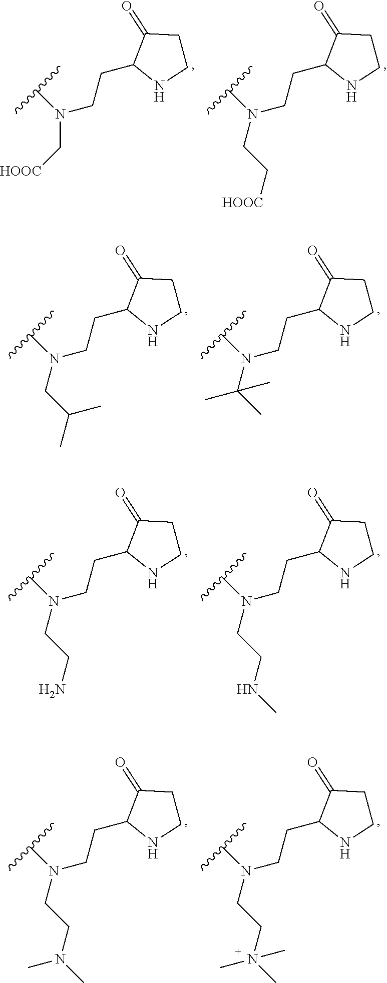

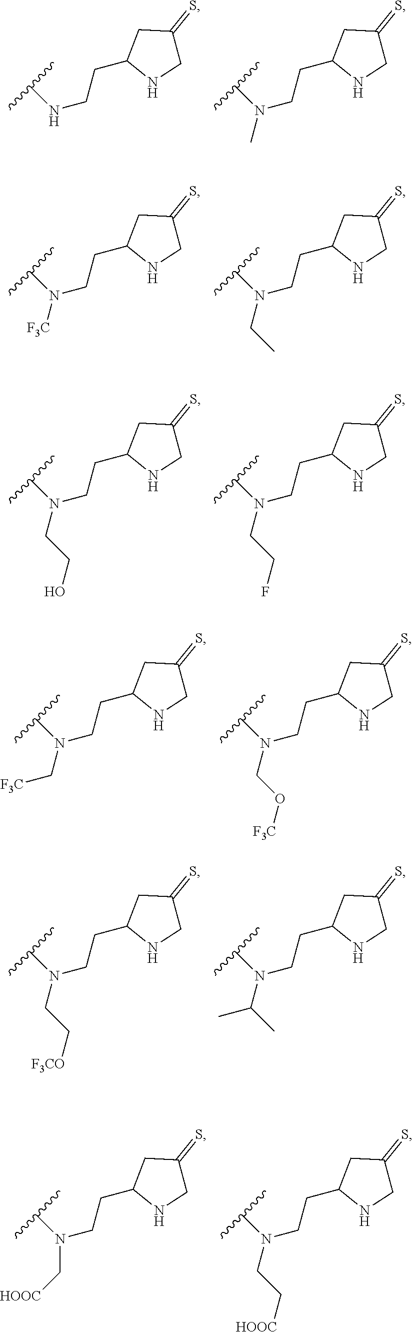

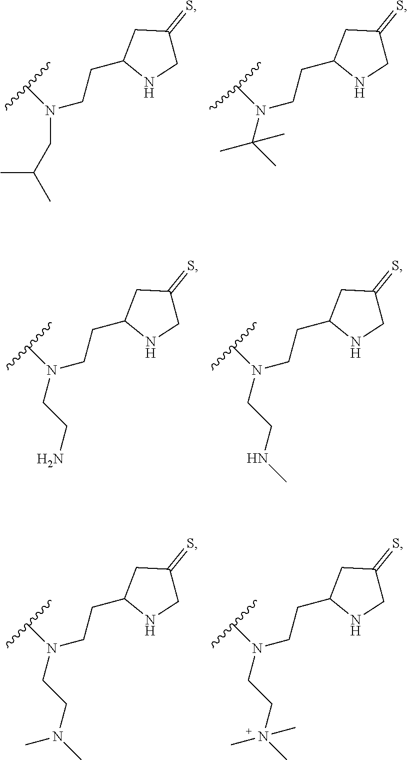

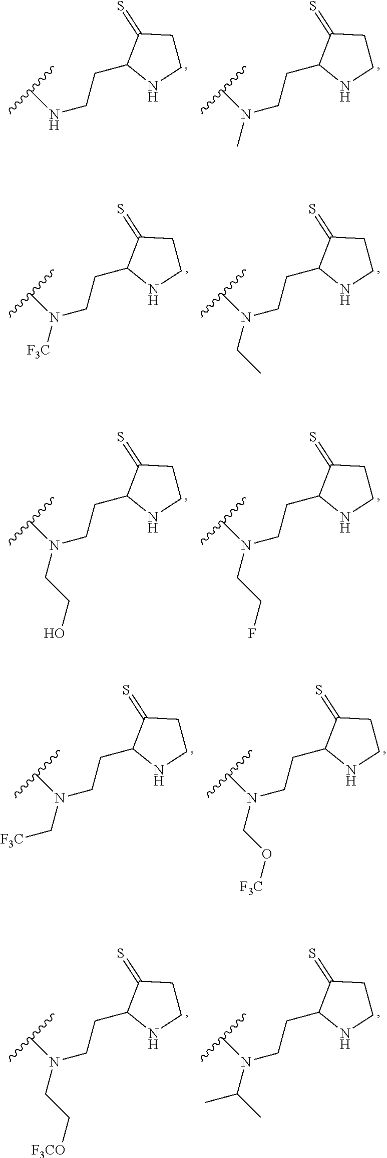

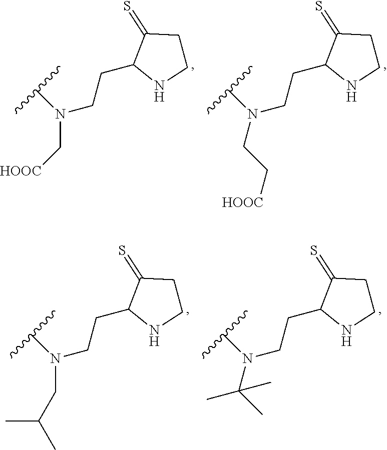 [SMILES: *N(CCC1NCCC1=S)CC(=O)O ; *N(CCC(=O)O)CCC1NCCC1=S ; *N(CCC1NCCC1=S)CC(C)C ; *N(CCC1NCCC1=S)C(C)(C)C]

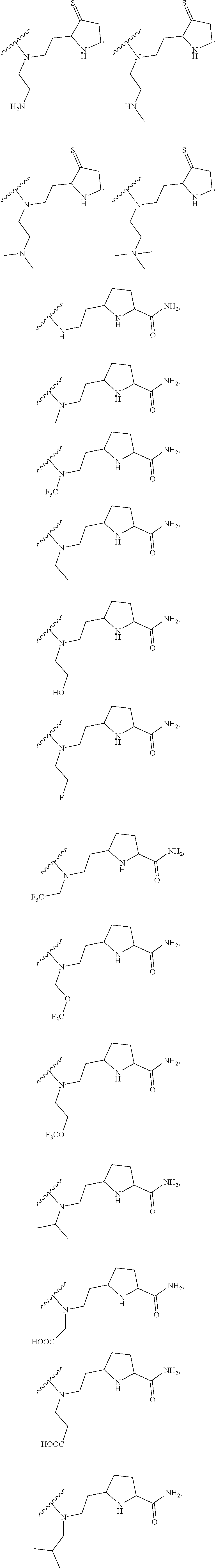

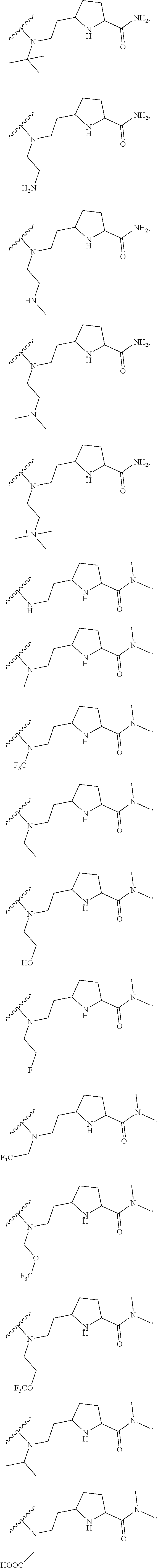

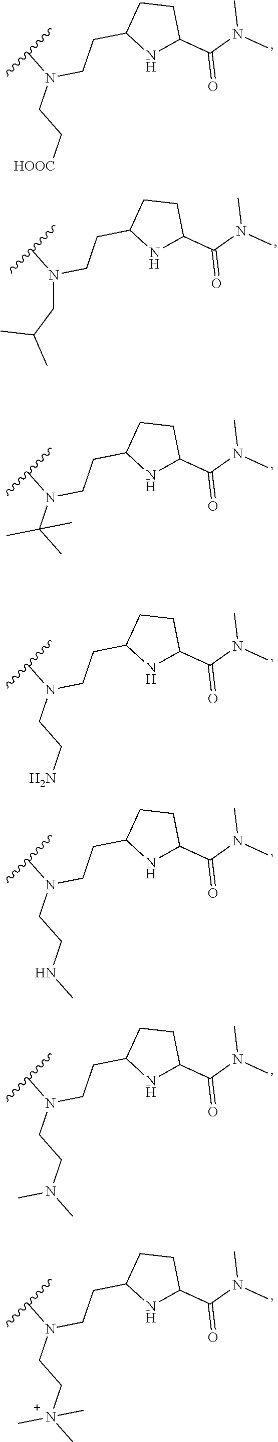

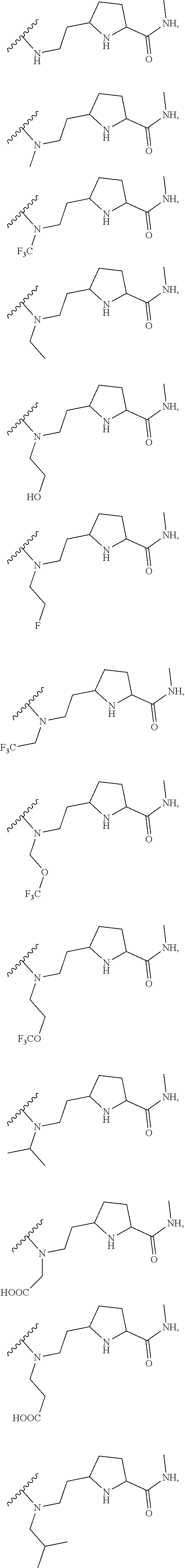

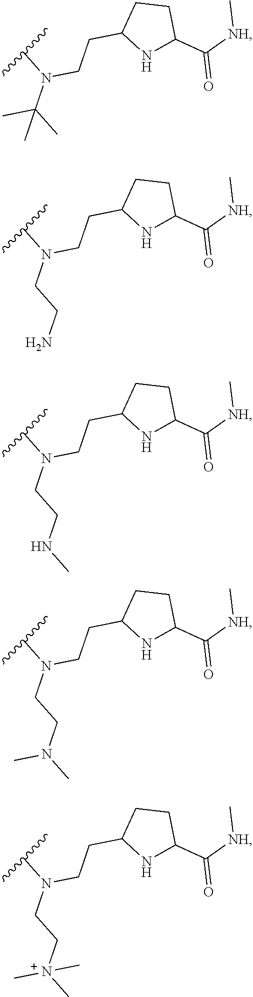 [SMILES: *N(CCC1CCC(C(=O)NC)N1)C(C)(C)C ; *N(CCN)CCC1CCC(C(=O)NC)N1 ; *N(CCNC)CCC1CCC(C(=O)NC)N1 ; *N(CCC1CCC(C(=O)NC)N1)CCN(C)C ; *N(CCC1CCC(C(=O)NC)N1)CC[N+](C)(C)C]

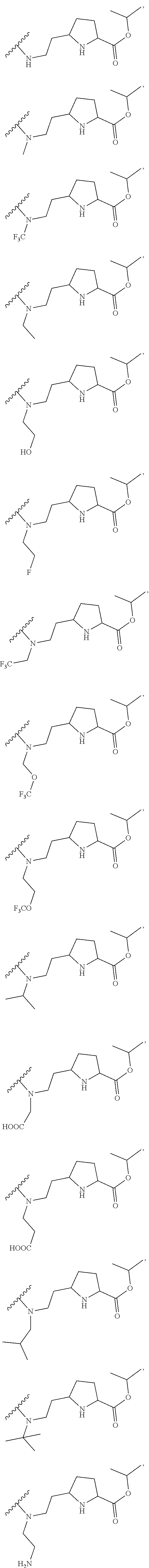

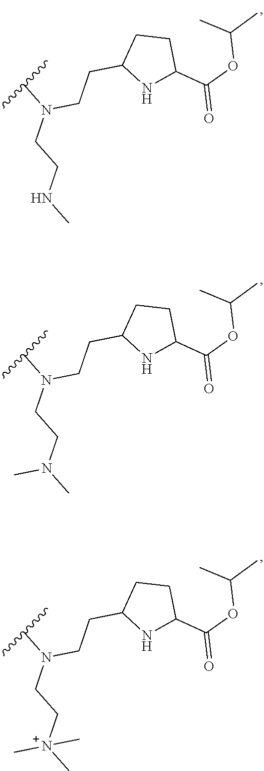

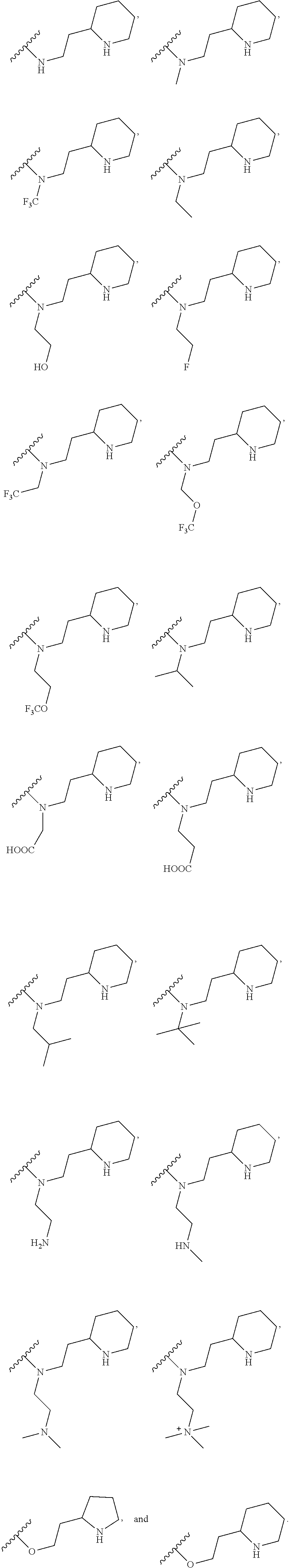

In some embodiments, the ring carbon atom in the pyrrolidine or piperidine moiety that connects to the rest of

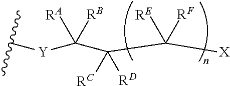

is in the S configuration. In some embodiments, the ring carbon atom in the pyrrolidine or piperidine moiety that connects to the rest of

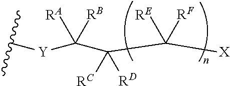

is in the R configuration.

In some embodiments, the

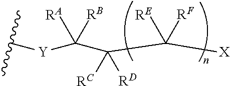

moiety in the prodrugs of Group II is selected from:

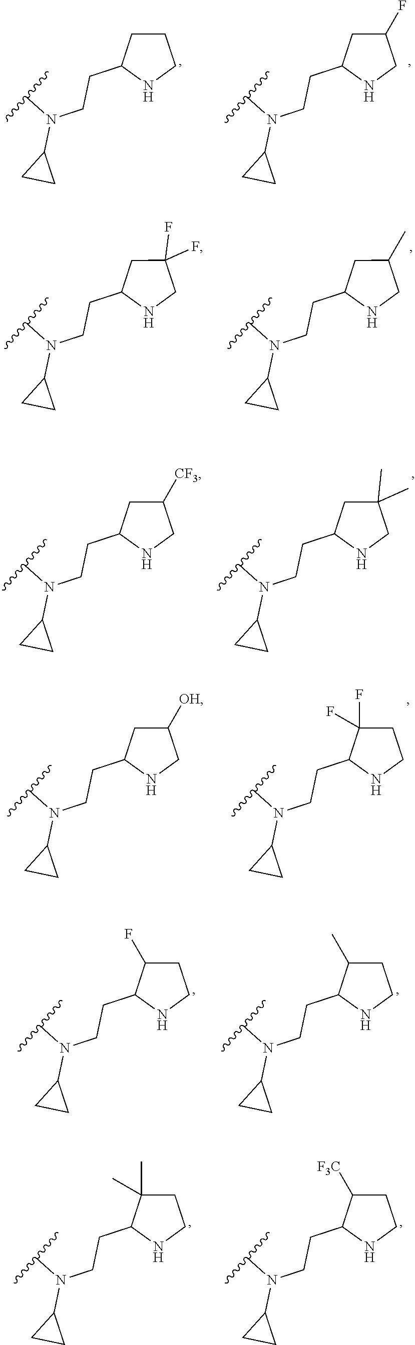

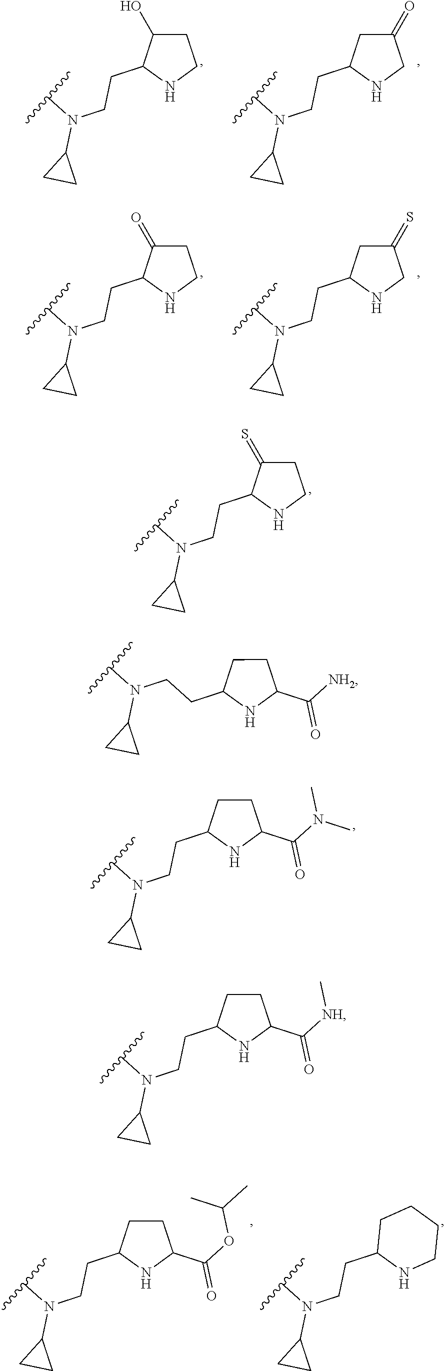

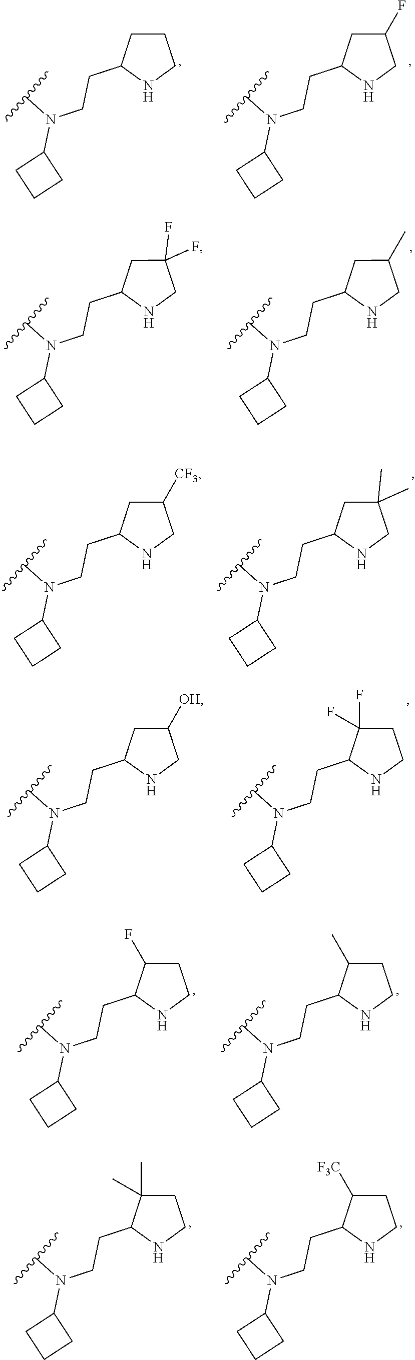

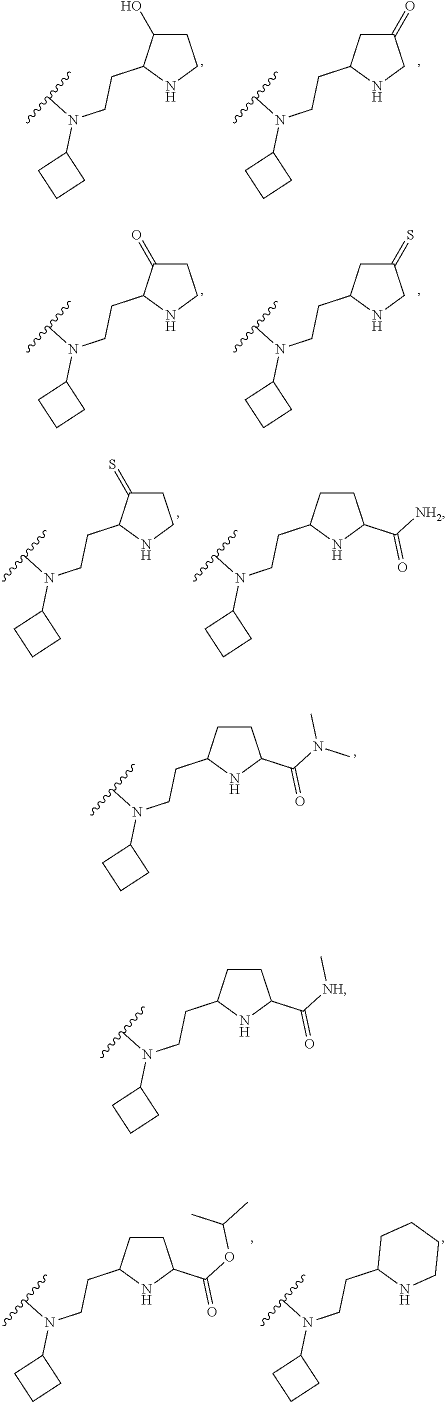

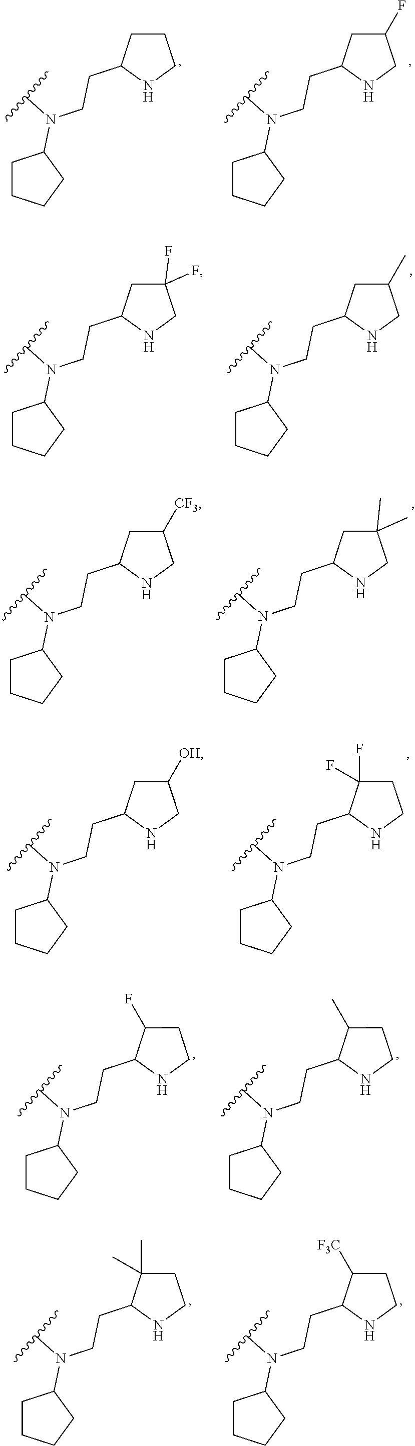

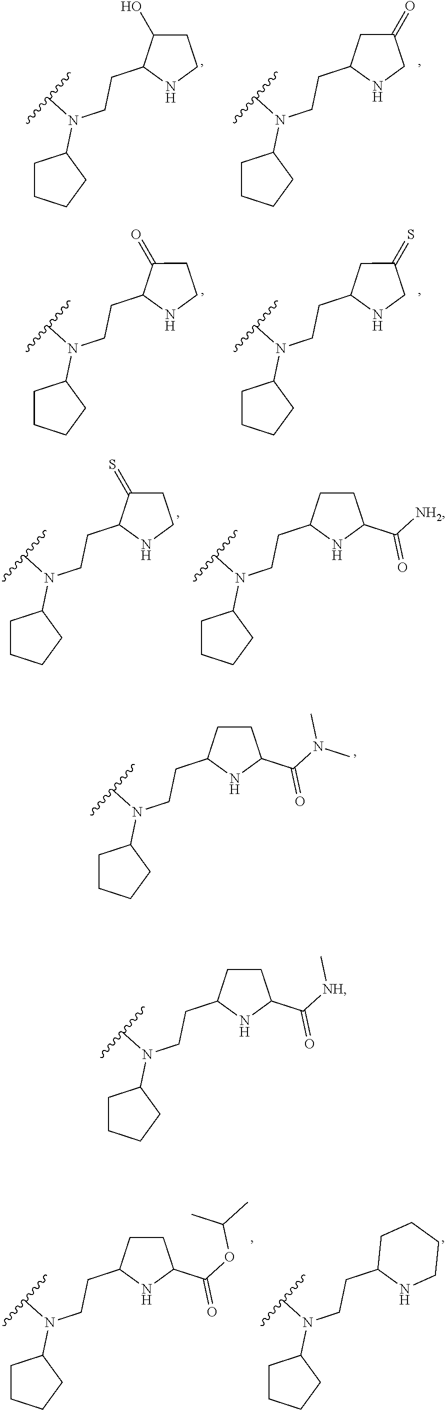

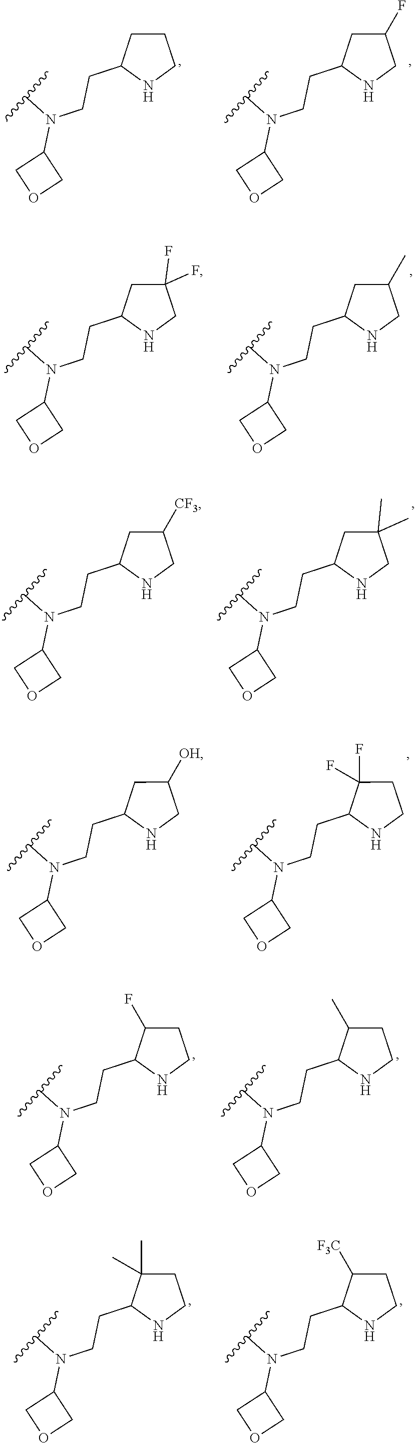

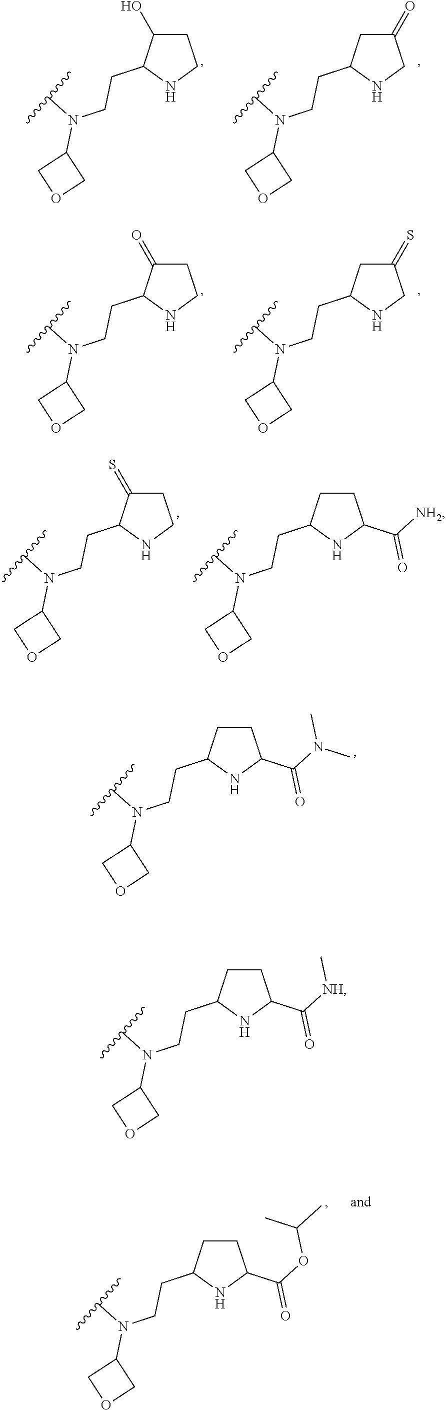

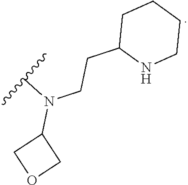 [SMILES: *N(CCC1CCCCN1)C1COC1]

In some embodiments, the ring carbon atom in the pyrrolidine or piperidine moiety that connects to the rest of

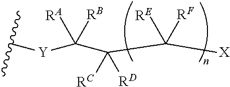

is in the S configuration. In some embodiments, the ring carbon atom in the pyrrolidine or piperidine moiety that connects to the rest of

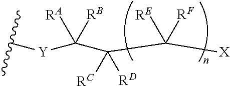

is in the R configuration.

In some embodiments, the

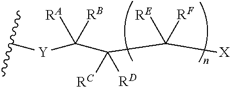

moiety in the prodrugs of Group II is selected from:

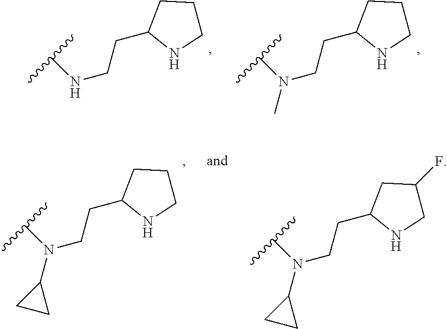

In some embodiments, the ring carbon atom in the pyrrolidine moiety that connects to the rest of

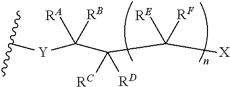

is in the S configuration. In some embodiments, the ring carbon atom in the pyrrolidine moiety that connects to the rest of

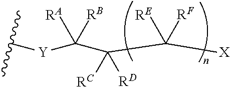

is in the R configuration.

### Group III

In some embodiments, the prodrugs have the following features:
X is OH or NR¹R²
Y is NR³,
R³ joins R^{A} to form a 4-7 membered, optionally substituted heterocycle,
R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹ and R² are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

In some embodiments, the 4-7 membered, optionally substituted heterocycle formed by R³ joining R^{A} is unsubstituted.

In some embodiments, the 4-7 membered, optionally substituted heterocycle formed by R³ joining R^{A} is substituted. Suitable substituents are in accordance with the general description of substitution in previous sections. In some embodiments, the 4-7 membered, optionally substituted heterocycle is substituted by one or more substituents independently selected from halogen, hydroxyl, ═O (counting as two substituents), ═S (counting as two substituents), cyano, nitro, carboxyl, carbonate, alkyl, haloalkyl, carbocyclyl, halocarbocyclyl, heterocyclyl, haloheterocyclyl, aryl, haloaryl, acyl, sulfinyl, sulfonyl, sulfonate, sulfonamide, amide, optionally Si-substituted silyl, ester, thioester, carbonate ester, and carbamate. In some embodiments, the 4-7 membered, optionally substituted heterocycle is substituted by one or more substituents independently selected from halogen, hydroxyl, cyano, alkyl, polyfluoroalkyl, ═O, ═S, amide, ester, sulfinyl, sulfonyl, sulfonate, and sulfonamide. In some embodiments, the 4-7 membered, optionally substituted heterocycle is substituted by one or more substituents independently selected from halogen, hydroxyl, ═O, ═S, amide, ester, alkyl, and polyfluoroalkyl. In some embodiments, the 4-7 membered, optionally substituted heterocycle is substituted by one or more substituents independently selected from fluorine, hydroxyl, ═O, ═S, —C(═O)NH₂, —C(═O)NHCH₃, —C(═O)N(CH₃)₂, —C(═O)OCH(CH₃)₂, methyl, and trifluoromethyl. In some embodiments, the 4-7 membered, optionally substituted heterocycle is substituted by one or more fluorine atoms. In some embodiments, the 4-7 membered, optionally substituted heterocycle is substituted by a —C(═O)NH₂ group. In some embodiments, the 4-7 membered, optionally substituted heterocycle is substituted by a —C(═O)OCH(CH₃)₂ group. In some embodiments, the 4-7 membered, optionally substituted heterocycle is substituted by a ═O group.

In some embodiments, the 4-7 membered, optionally substituted heterocycle is a 5 or 6 membered, optionally substituted heterocycle. In some embodiments, the 4-7 membered, optionally substituted heterocycle is a 5-membered, optionally substituted heterocycle. In some embodiments, the 4-7 membered, optionally substituted heterocycle is a 6-membered, optionally substituted heterocycle. In some embodiments, the 4-7 membered, optionally substituted heterocycle is optionally substituted pyrrolidine or optionally substituted piperidine. In some embodiments, the 4-7 membered, optionally substituted heterocycle is optionally substituted pyrrolidine. In some embodiments, the 4-7 membered, optionally substituted heterocycle is pyrrolidine or piperidine. In some embodiments, the 4-7 membered, optionally substituted heterocycle is pyrrolidine.

In some embodiments, X is OH. In some embodiments, X is NR¹R². In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R² is hydrogen. In some embodiments, R¹ is —CH₃. and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃. and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃⁺, and R² is hydrogen.

In some embodiments, n is 0, i.e., both R^{E} and R^{F} are absent. In some embodiments, n is 1, i.e., both R^{E} and R^{F} are present.

Optionally, R^{B} is hydrogen.

Optionally, R^{B} is optionally substituted alkyl. In some embodiments, R^{B} is optionally substituted C₁-C₄ alkyl. In some embodiments, R^{B} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R^{B} is methyl.

Optionally, at least one of R^{C} and R^{D} is hydrogen. In some embodiments, both R^{C} and R^{D} are hydrogen. In some embodiments, R^{e} is optionally substituted alkyl, and R^{D} is hydrogen. In some embodiments, R^{e} is optionally substituted C₁-C₄ alkyl, and R^{D} is hydrogen. In some embodiments, R^{e} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{D} is hydrogen. In some embodiments, R^{C} is methyl, and R^{D} is hydrogen.

Optionally, at least one of R^{C} and R^{D} is optionally substituted alkyl. In some embodiments, at least one of R^{C} and R^{D} is optionally substituted C₁-C₄ alkyl. In some embodiments, at least one of R^{C} and R^{D} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{C} and R^{D} is methyl. In some embodiments, each of R^{C} and R^{D} is, independently, an optionally substituted alkyl. In some embodiments, each of R^{C} and R^{D} is, independently, an optionally substituted C₁-C₄ alkyl. In some embodiments, each of R^{C} and R^{D} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{C} and R^{D} is methyl.

In some embodiments, R^{B}, R^{C}, and R^{D} are hydrogen.

Optionally, at least one of R^{E} and R^{F} is hydrogen. In some embodiments, both R^{E} and R^{F} are hydrogen. In some embodiments, R^{E} is optionally substituted alkyl, and R^{F} is hydrogen. In some embodiments, R^{E} is optionally substituted C₁-C₄ alkyl, and R^{F} is hydrogen. In some embodiments, R^{E} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{F} is hydrogen. In some embodiments, R^{E} is methyl, and R^{F} is hydrogen.

Optionally, at least one of R^{E} and R^{F} is optionally substituted alkyl. In some embodiments, at least one of R^{E} and R^{F} is optionally substituted C₁-C₄ alkyl. In some embodiments, at least one of R^{E} and R^{F} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{E} and R^{F} is methyl. In some embodiments, each of R^{E} and R^{F} is, independently, an optionally substituted alkyl. In some embodiments, each of R^{E} and R^{F} is, independently, an optionally substituted C₁-C₄ alkyl. In some embodiments, each of R^{E} and R^{F} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{E} and R^{F} is methyl.

In some embodiments, n is 0, X is NR¹R², and Y is NR³. In some embodiments, n is 0, X is NR¹R², Y is NR³, and R^{B} is hydrogen. In some embodiments, n is 0, X is NR¹R², Y is NR³, and R^{B} is methyl. In some embodiments, n is 0, X is NR¹R², Y is NR³, and R^{C} and R^{D} are hydrogen. In some embodiments, n is 0, X is NR¹R², Y is NR³, R^{C} is methyl, and R^{D} is hydrogen. In some embodiments, n is 0, X is NR¹R², Y is NR³, and R^{C} and R^{D} are methyl. In some embodiments, n is 0, X is NR¹R², Y is NR³, and R^{B}, R^{C}, and R^{D} are hydrogen. In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃⁺, and R² is hydrogen.

In some embodiments, n is 1, X is NR¹R², and Y is NR³. In some embodiments, n is 1, X is NR¹R², Y is NR³, and R^{B} is hydrogen. In some embodiments, n is 1, X is NR¹R², Y is NR³, and R^{B} is methyl. In some embodiments, n is 1, X is NR¹R², Y is NR³, and R^{C} and R^{D} are hydrogen. In some embodiments, n is 1, X is NR¹R², Y is NR³, R^{C} is methyl, and R^{D} is hydrogen. In some embodiments, n is 1, X is NR¹R², Y is NR³, and R^{C} and R^{D} are methyl. In some embodiments, n is 1, X is NR¹R², Y is NR³, and R^{E} and R^{F} are hydrogen. In some embodiments, n is 1, X is NR¹R², Y is NR³, R^{E} is methyl, and R^{F} is hydrogen. In some embodiments, n is 1, X is NR¹R², Y is NR³, and R^{E} and R^{F} are methyl. In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃⁺, and R² is hydrogen.

In some embodiments, the

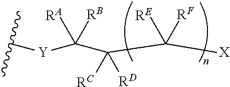

moiety in the prodrugs of Group III is selected from:

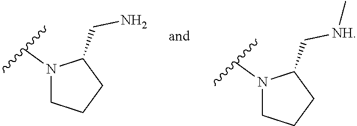

### 3. Exemplary Structures

In some embodiments, the prodrugs have a structure of Formula II or a pharmaceutically acceptable salt, hydrate, or hydrated salt thereof,

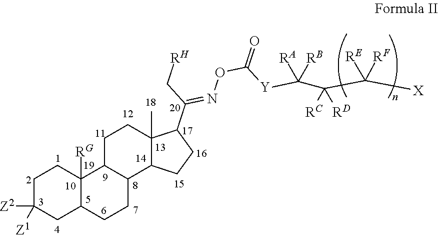

wherein the

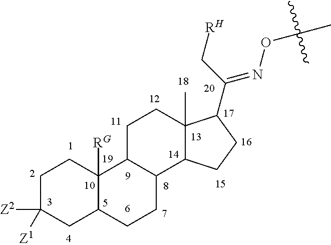

moiety is selected from:

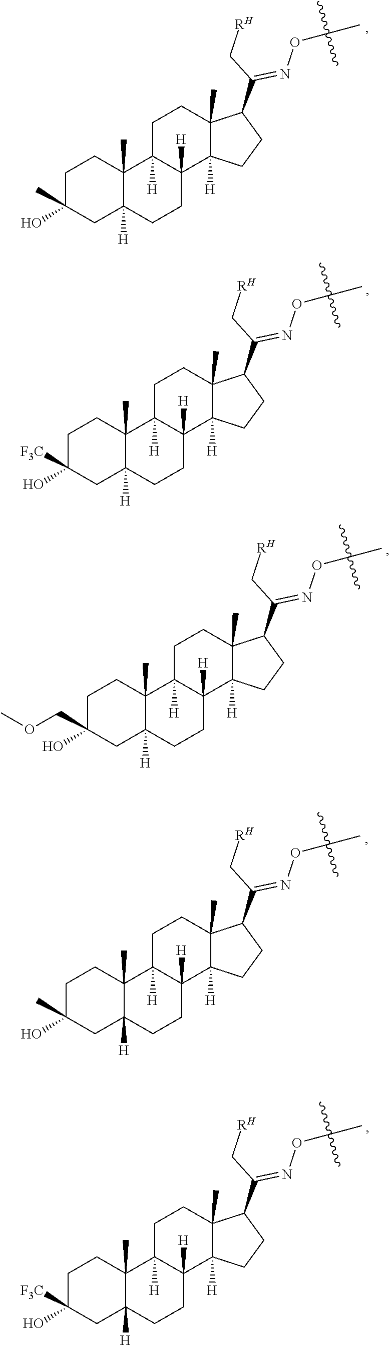

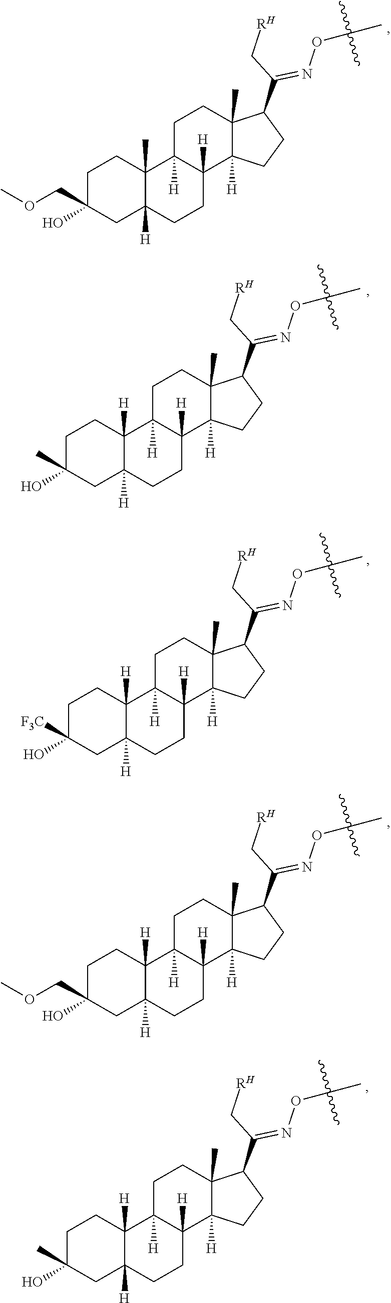

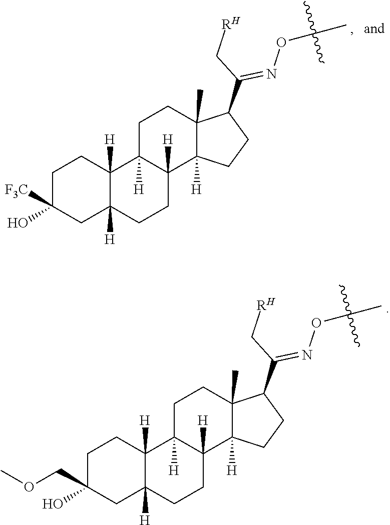

wherein R^{H} is selected from H,

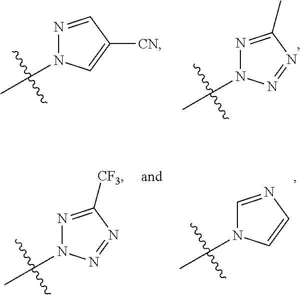

and
wherein the

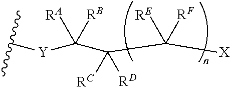

moiety is selected from:

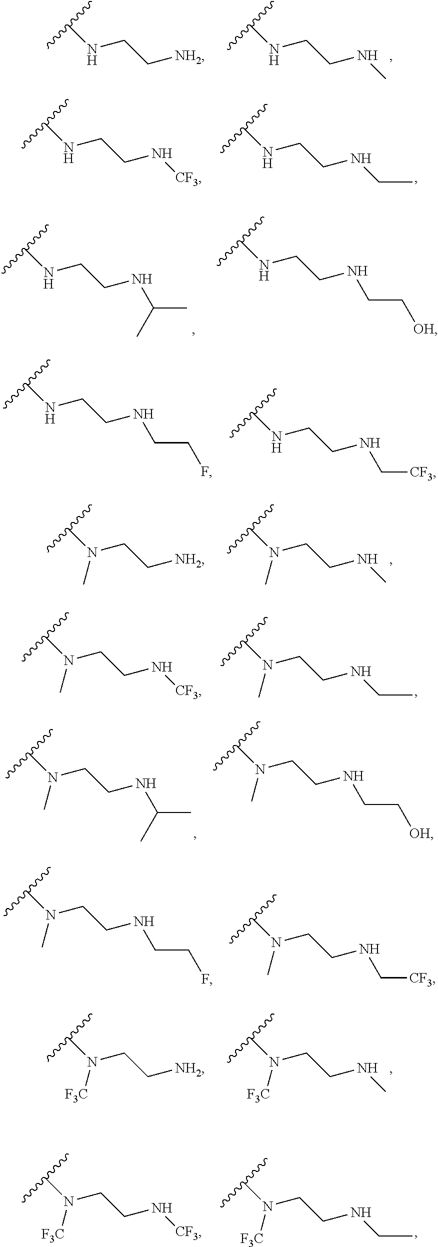

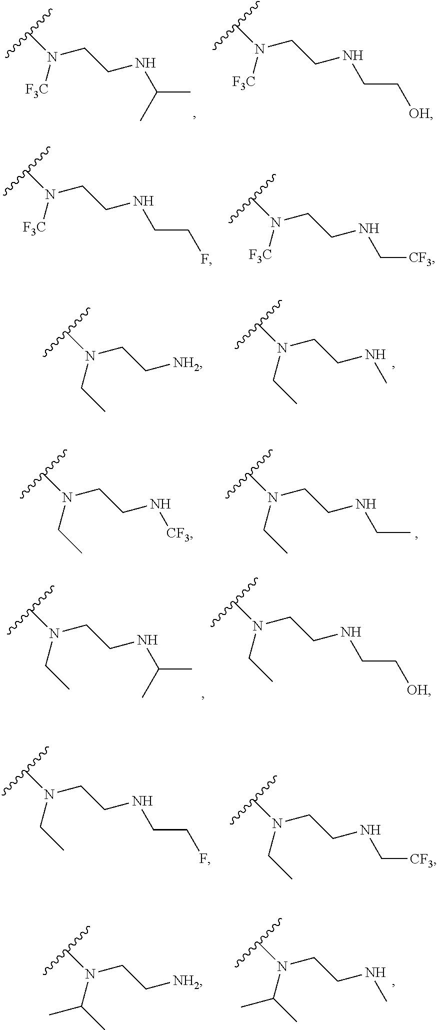

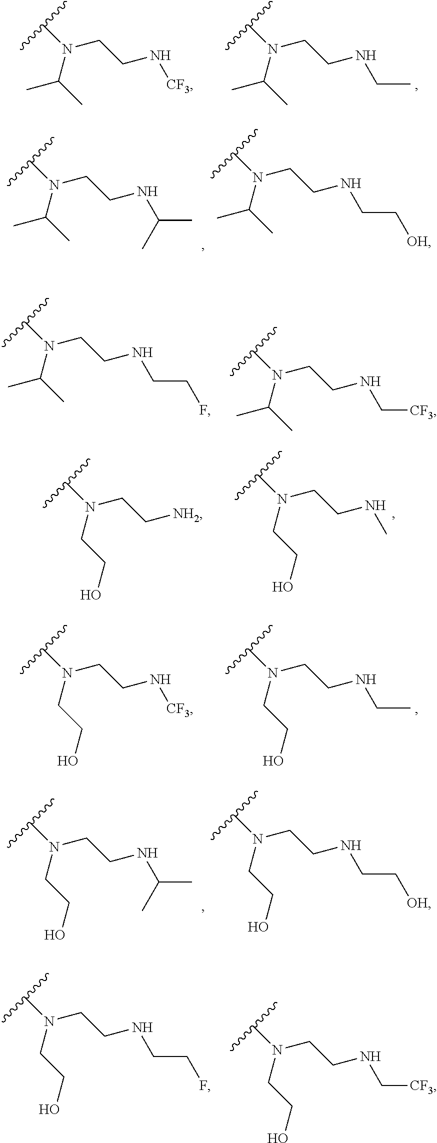

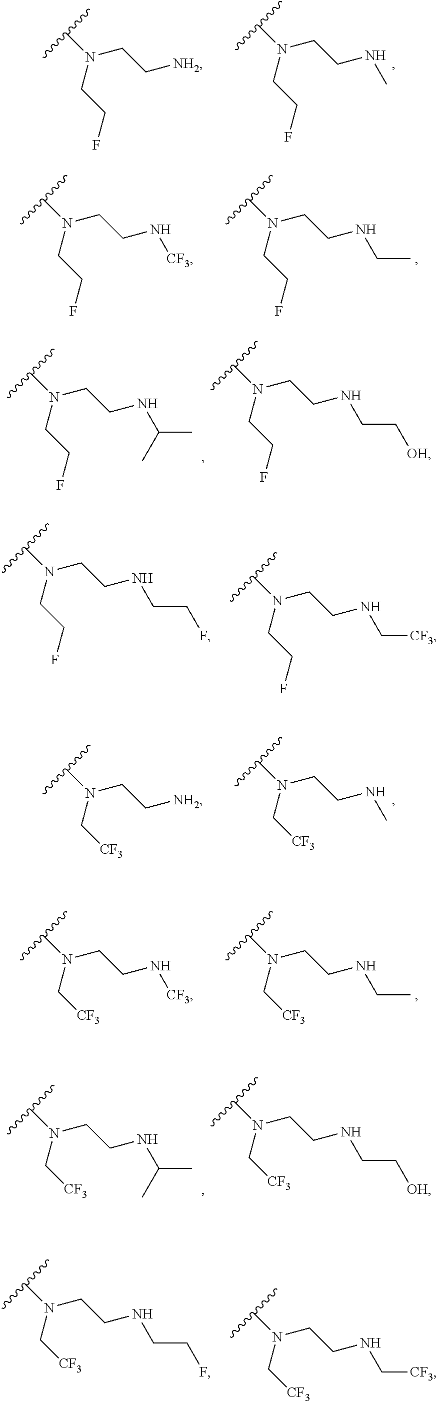

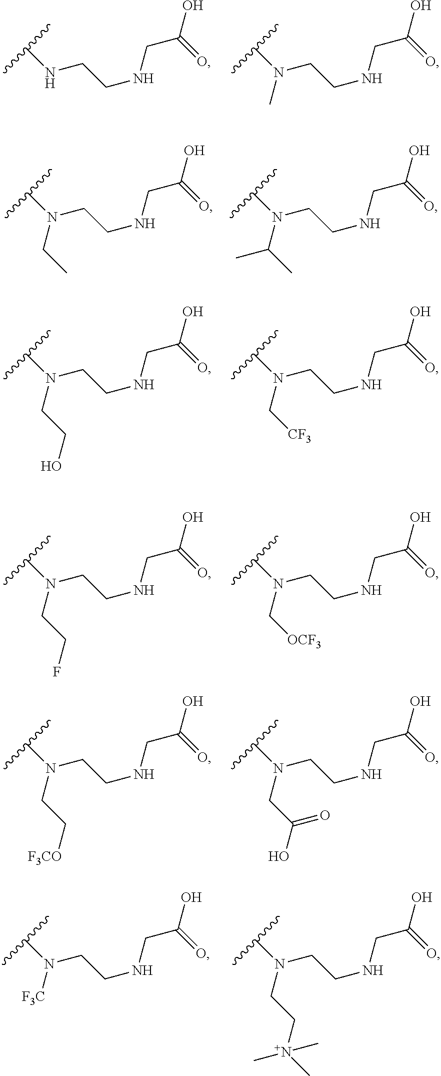

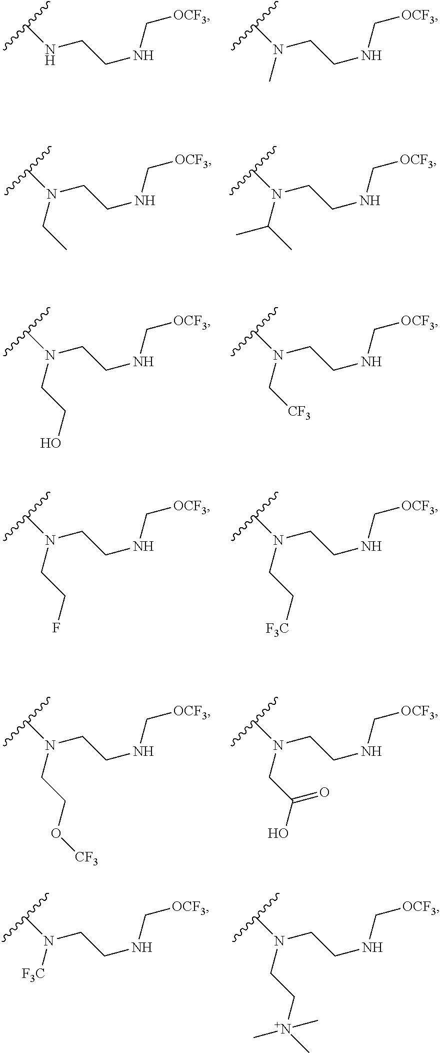

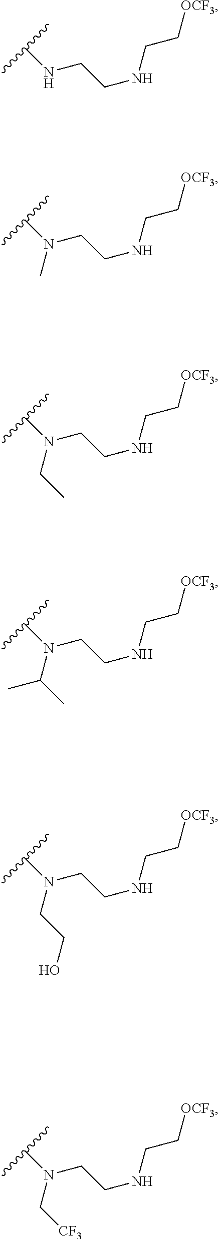 [SMILES: *NCCNCCOC(F)(F)F ; *N(C)CCNCCOC(F)(F)F ; *N(CC)CCNCCOC(F)(F)F ; *N(CCNCCOC(F)(F)F)C(C)C ; *N(CCO)CCNCCOC(F)(F)F ; *N(CCNCCOC(F)(F)F)CC(F)(F)F]

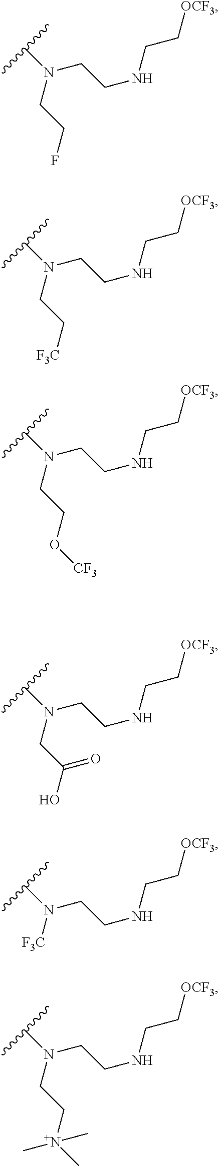 [SMILES: *N(CCF)CCNCCOC(F)(F)F ; *N(CCNCCOC(F)(F)F)CCC(F)(F)F ; *N(CCNCCOC(F)(F)F)CCOC(F)(F)F ; *N(CCNCCOC(F)(F)F)CC(=O)O ; *N(CCNCCOC(F)(F)F)C(F)(F)F ; *N(CCNCCOC(F)(F)F)CC[N+](C)(C)C]

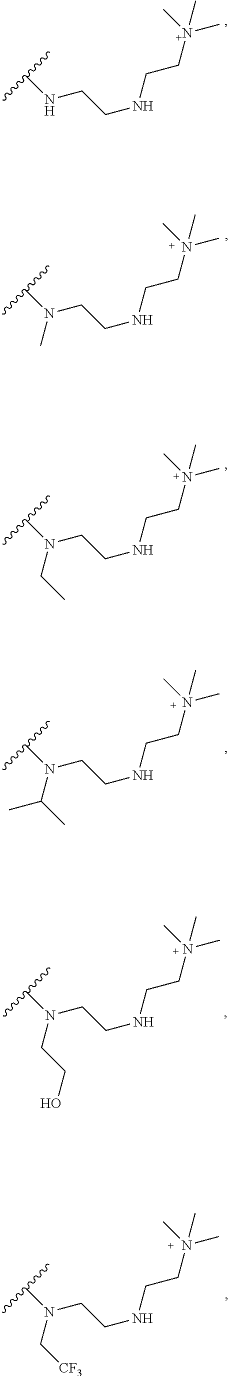

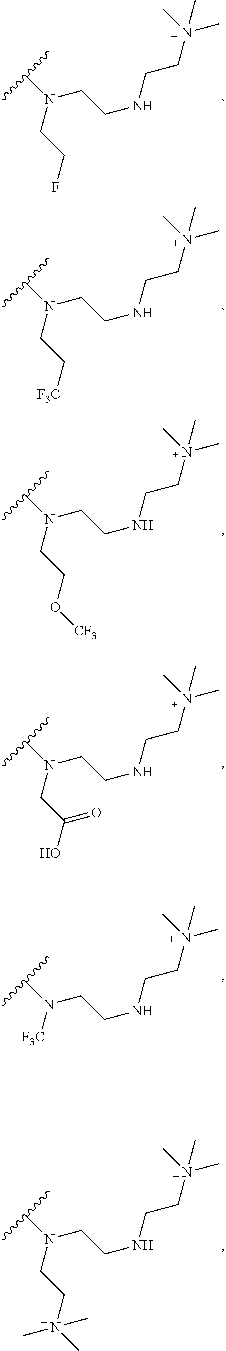

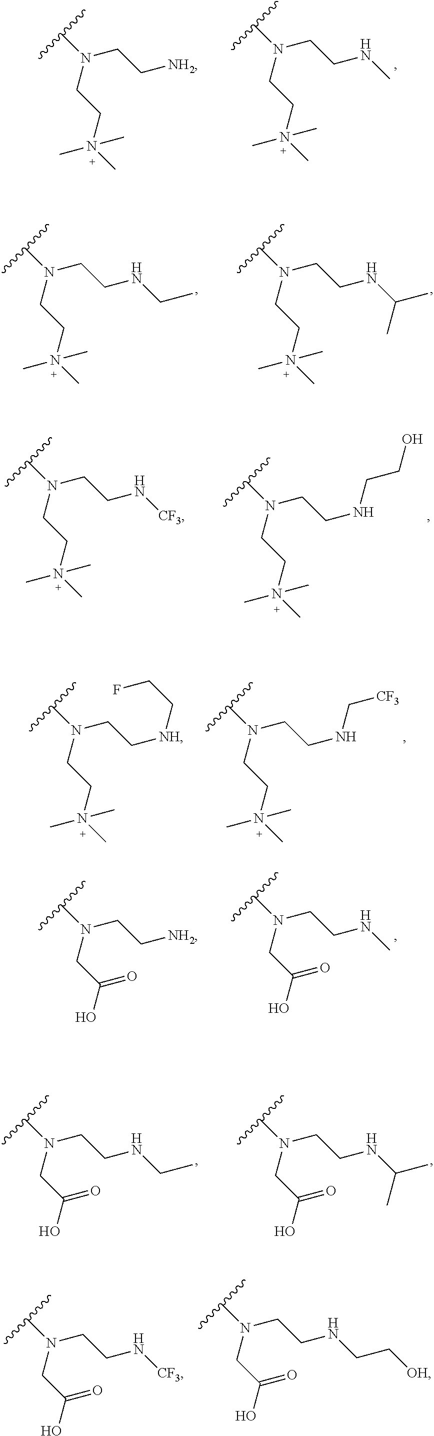

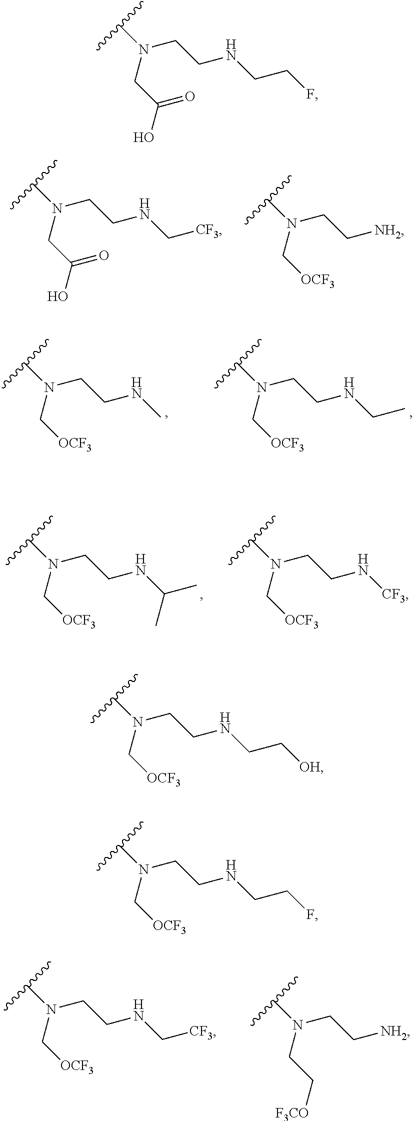

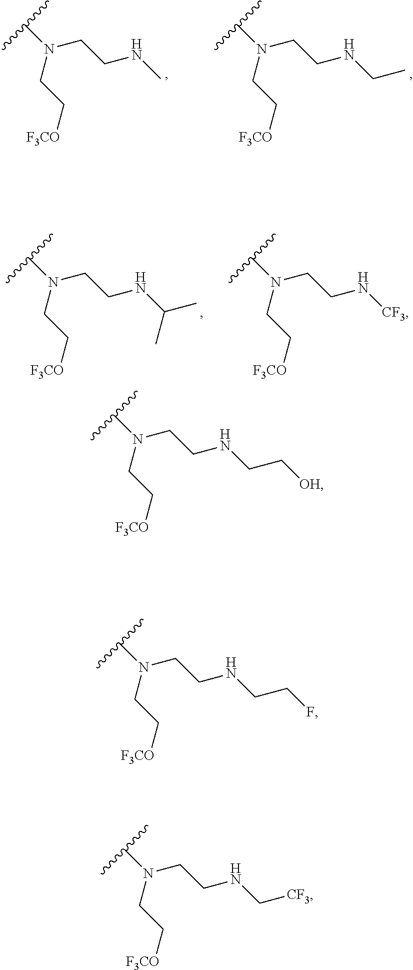

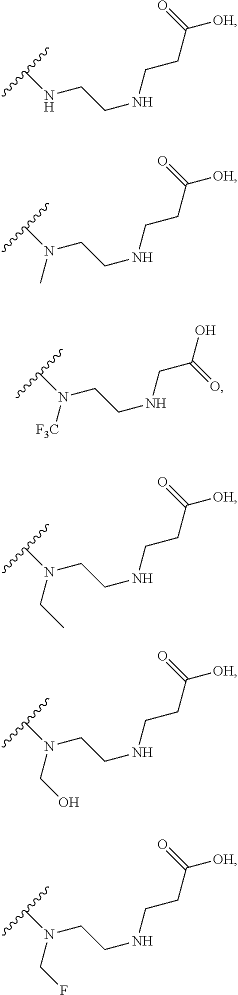 [SMILES: *NCCNCCC(=O)O ; *N(C)CCNCCC(=O)O ; *N(CCNCC(=O)O)C(F)(F)F ; *N(CC)CCNCCC(=O)O ; *N(CO)CCNCCC(=O)O ; *N(CF)CCNCCC(=O)O]

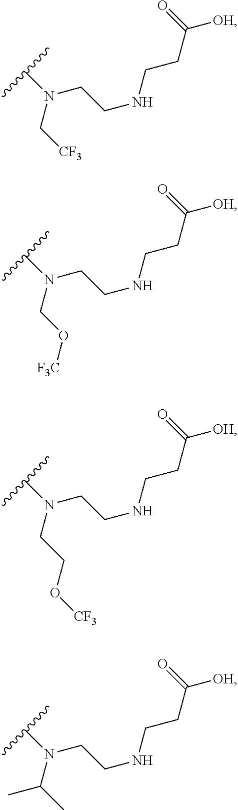 [SMILES: *N(CCNCCC(=O)O)CC(F)(F)F ; *N(CCNCCC(=O)O)COC(F)(F)F ; *N(CCNCCC(=O)O)CCOC(F)(F)F ; *N(CCNCCC(=O)O)C(C)C]

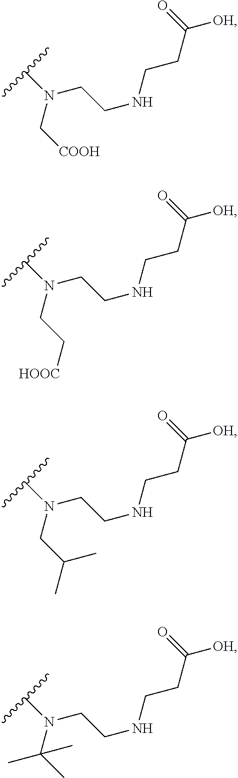 [SMILES: *N(CCNCCC(=O)O)CC(=O)O ; *N(CCNCCC(=O)O)CCC(=O)O ; *N(CCNCCC(=O)O)CC(C)C ; *N(CCNCCC(=O)O)C(C)(C)C]

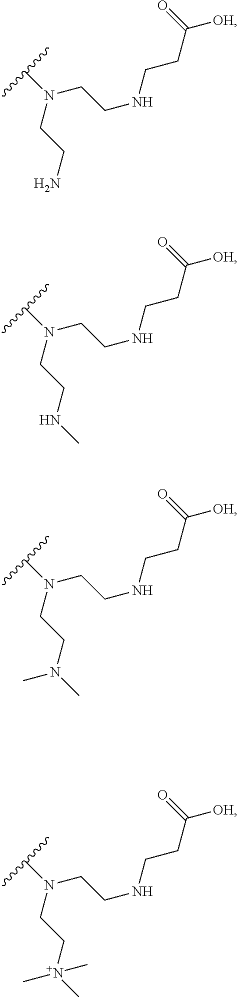 [SMILES: *N(CCN)CCNCCC(=O)O ; *N(CCNC)CCNCCC(=O)O ; *N(CCNCCC(=O)O)CCN(C)C ; *N(CCNCCC(=O)O)CC[N+](C)(C)C]

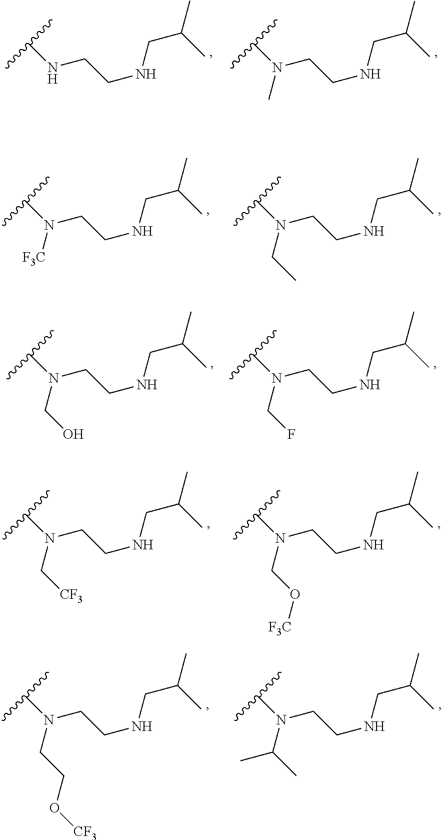

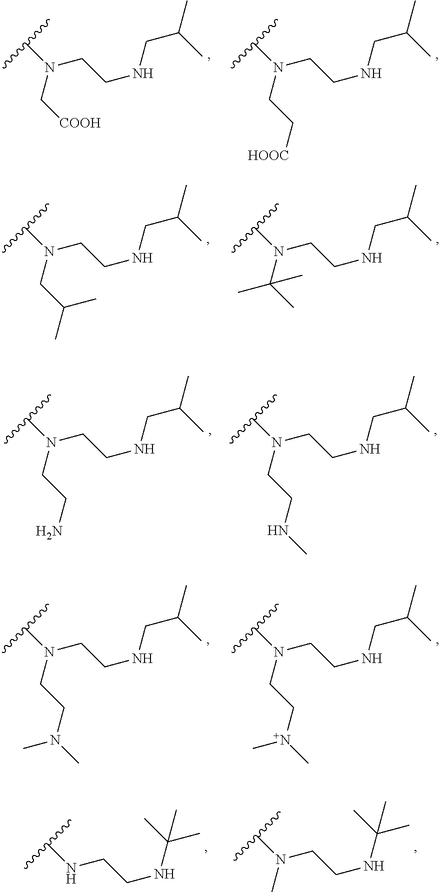

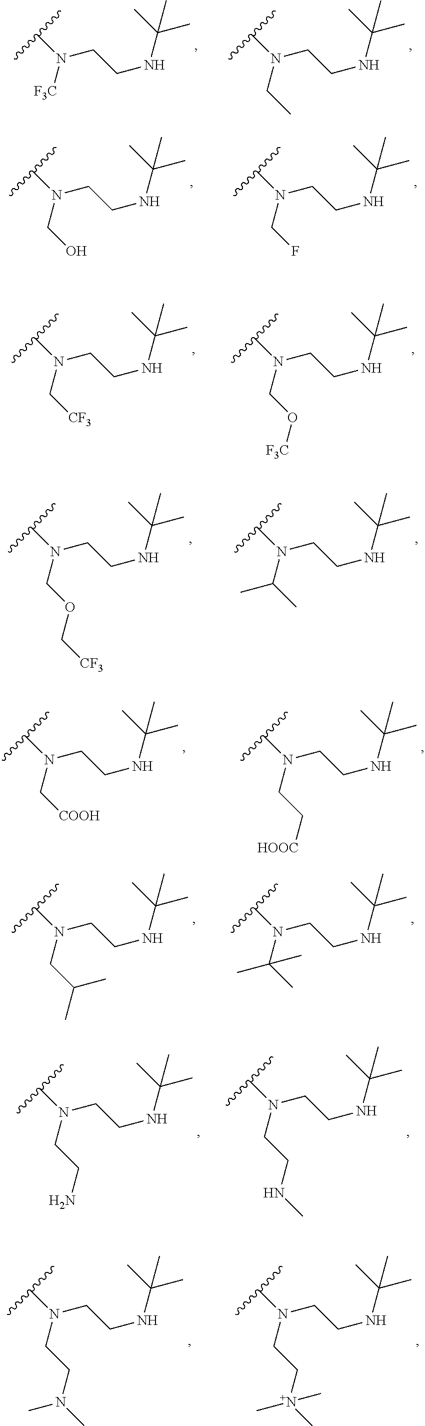

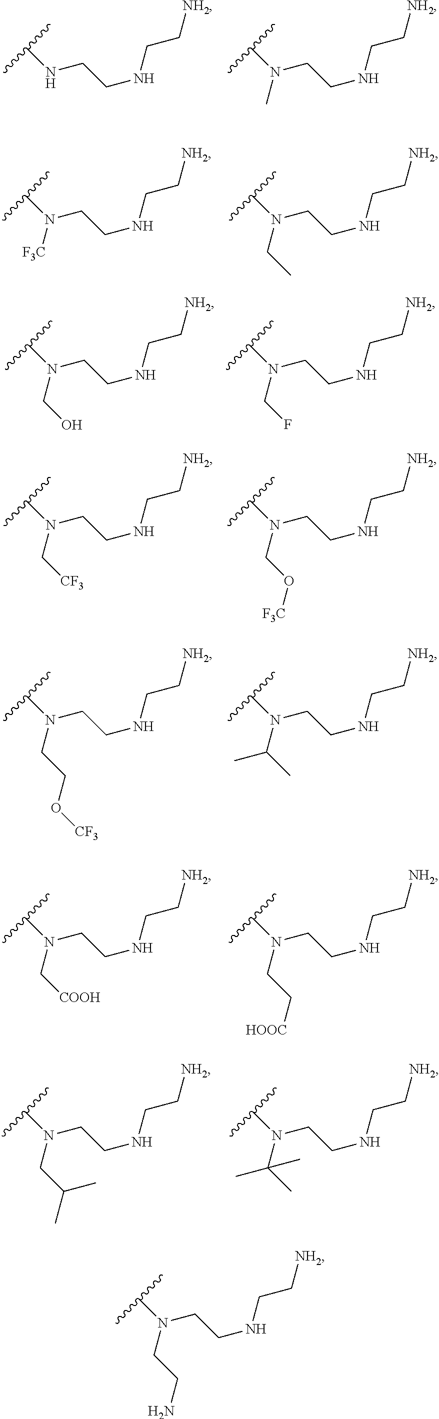

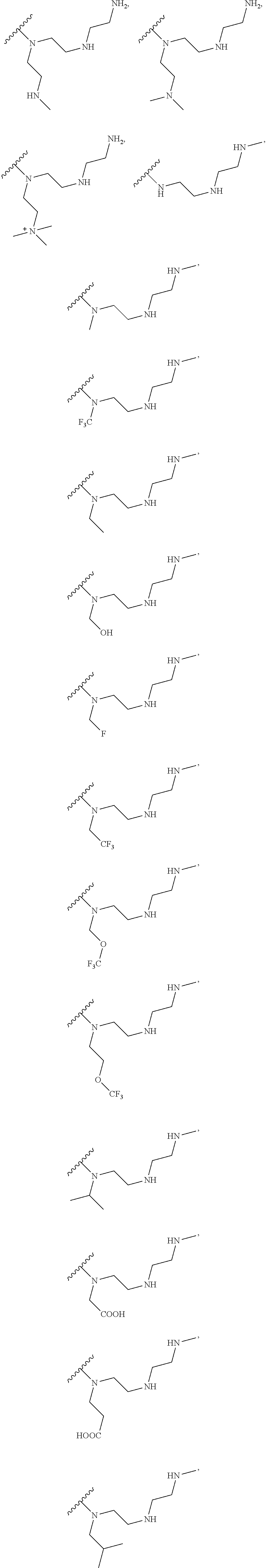

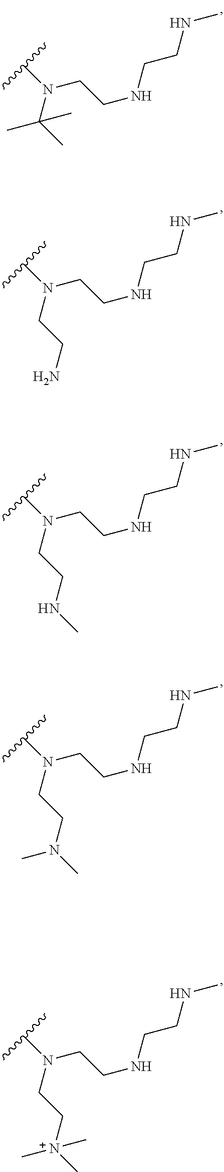

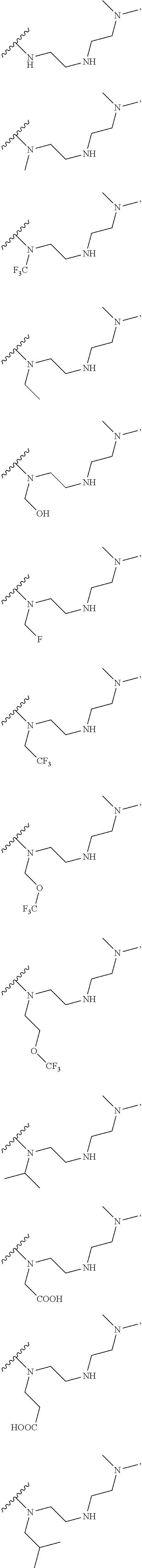

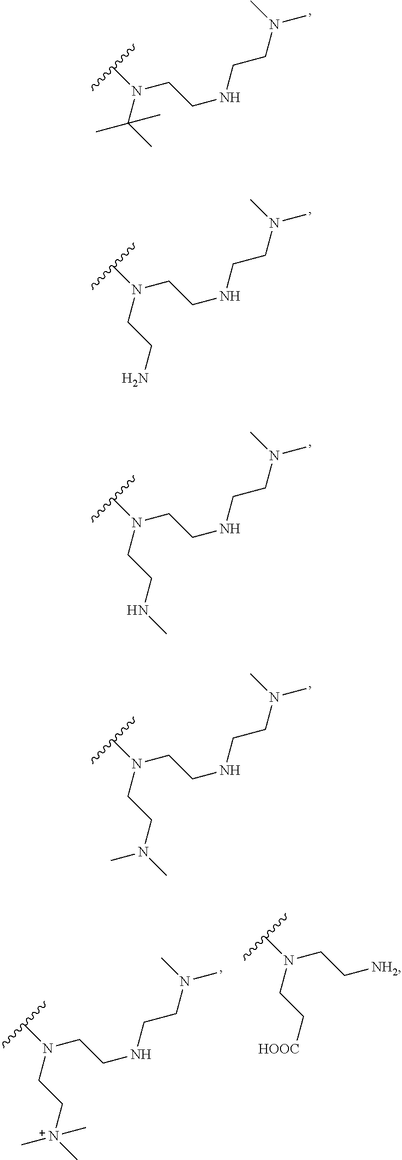

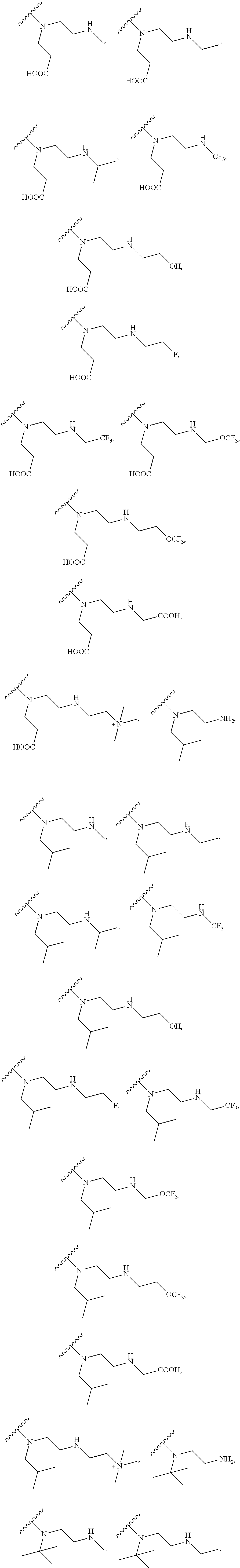

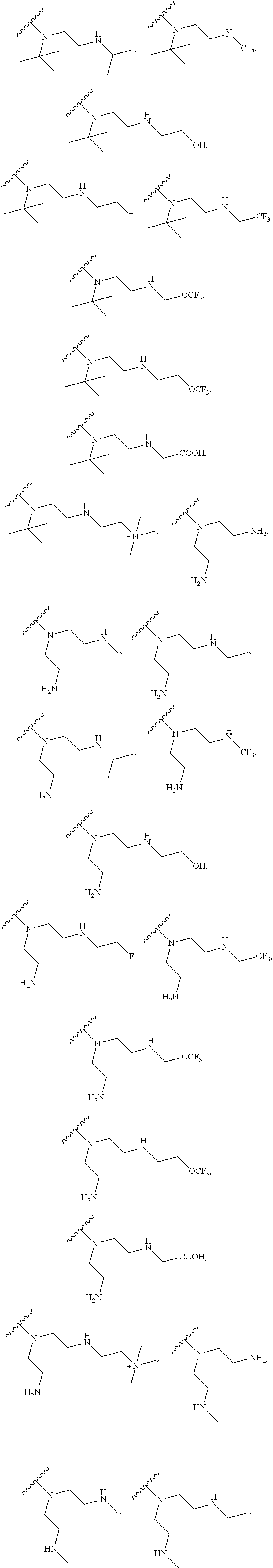

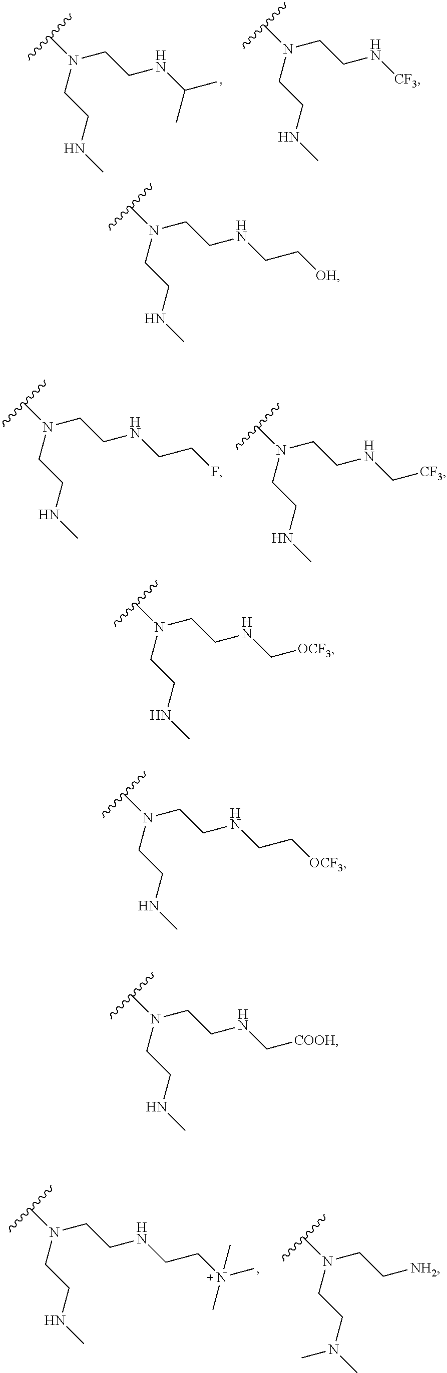

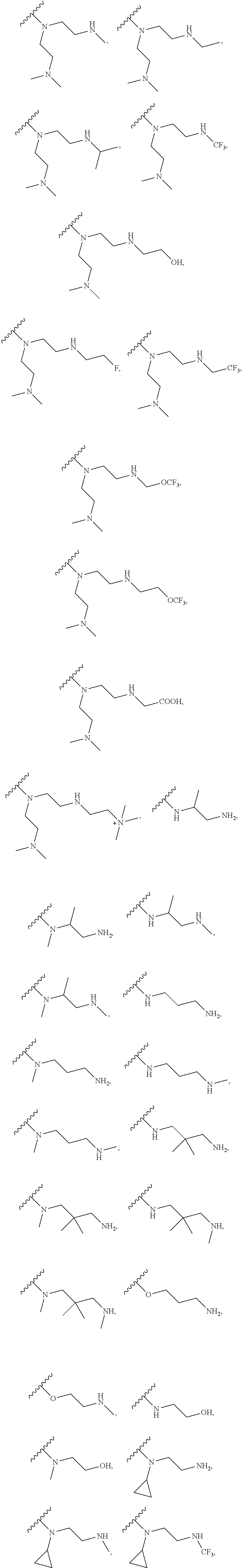

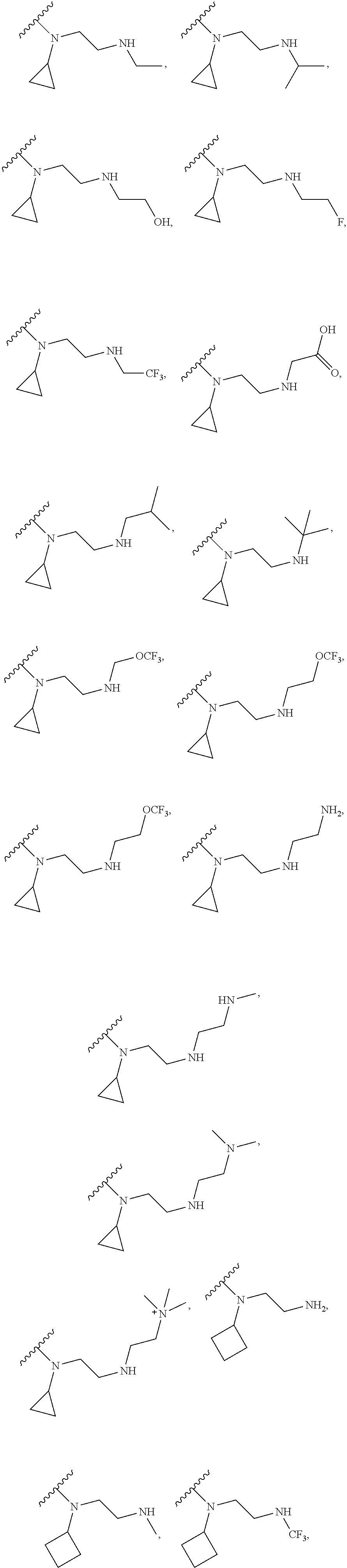

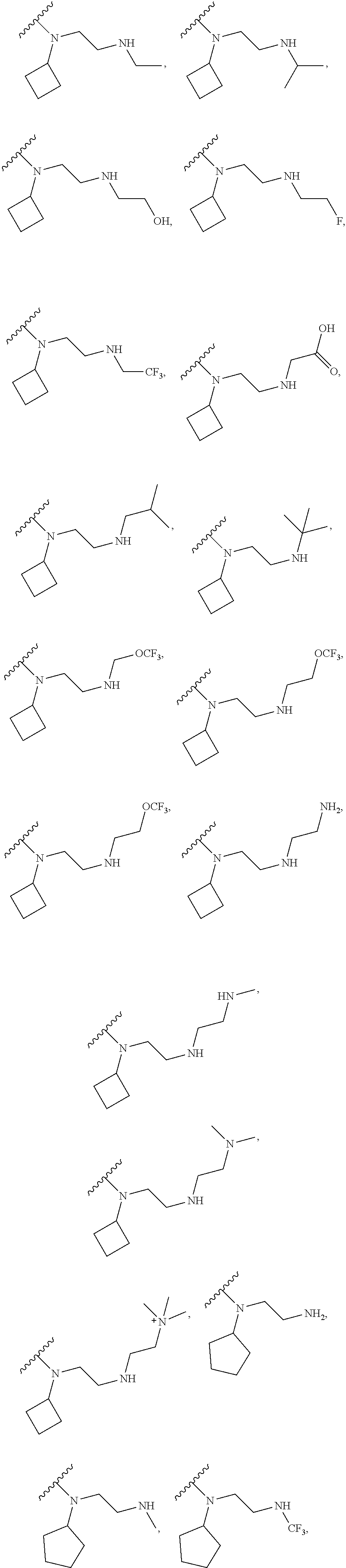

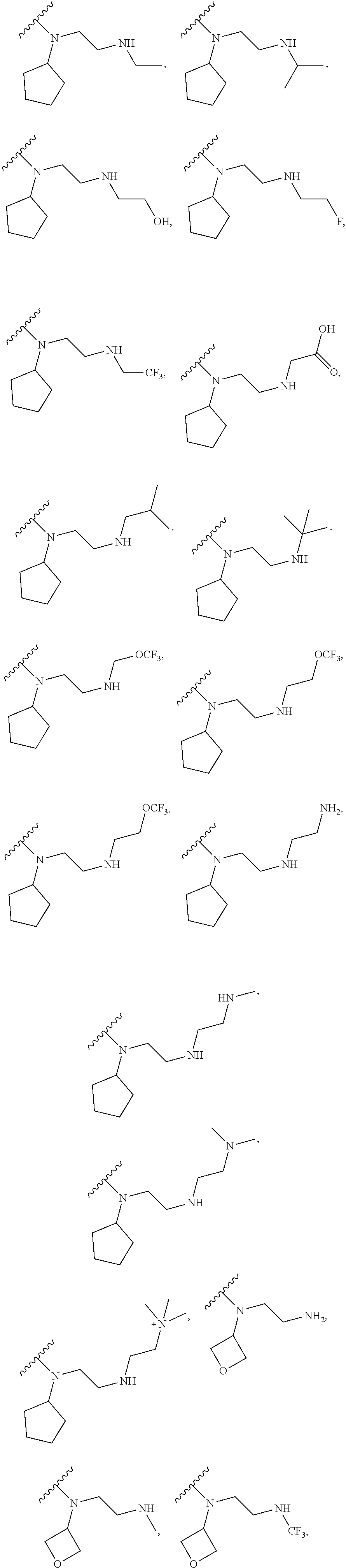

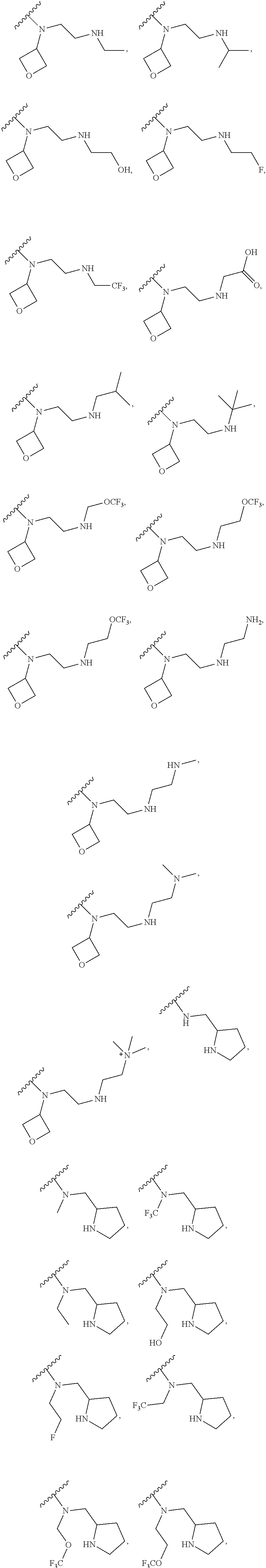

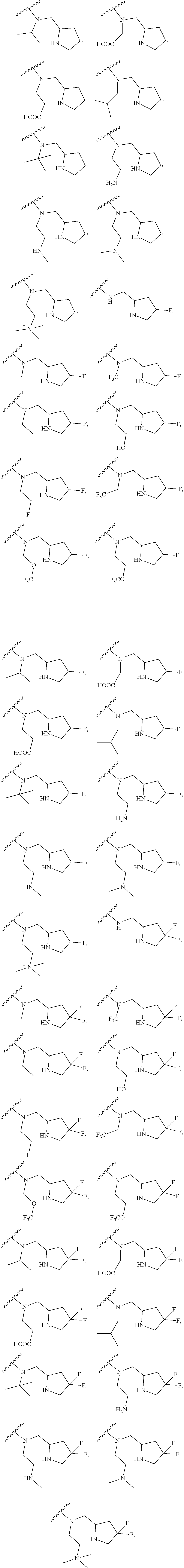

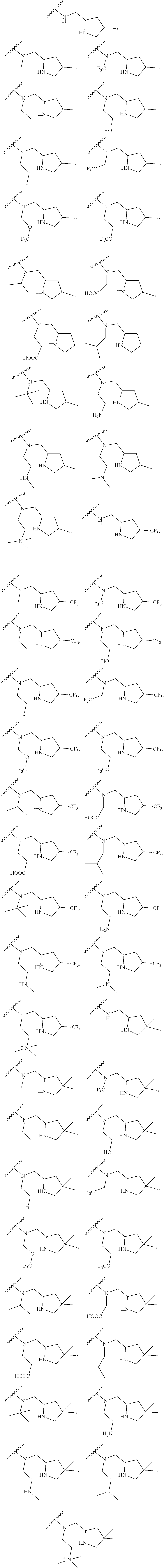

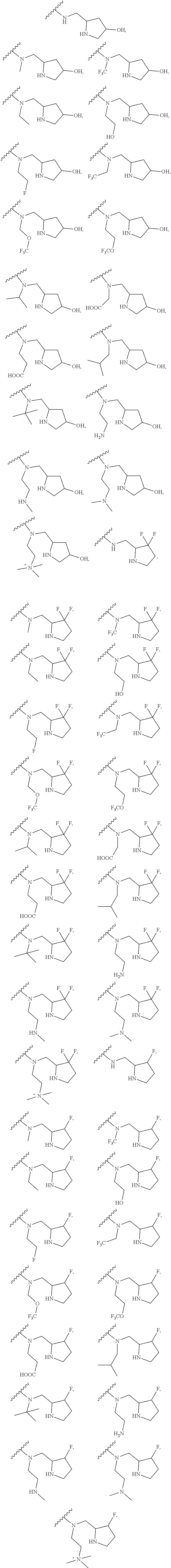

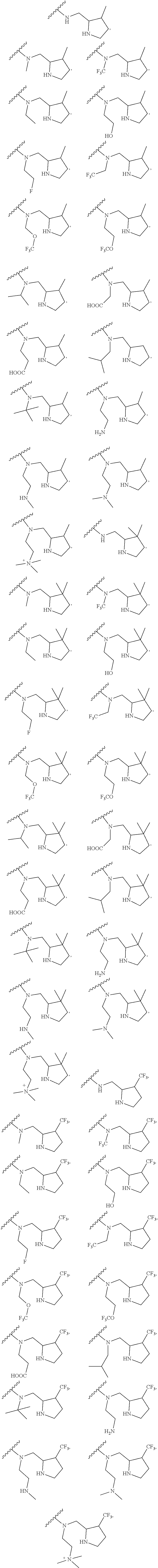

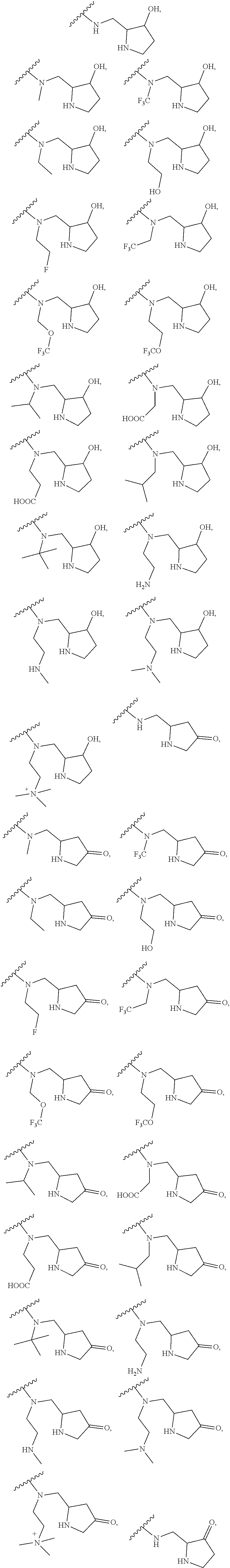

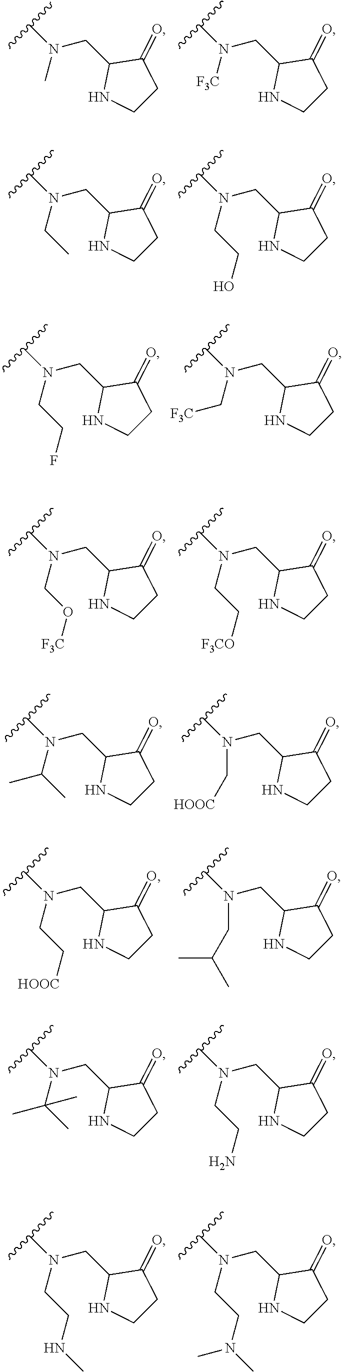

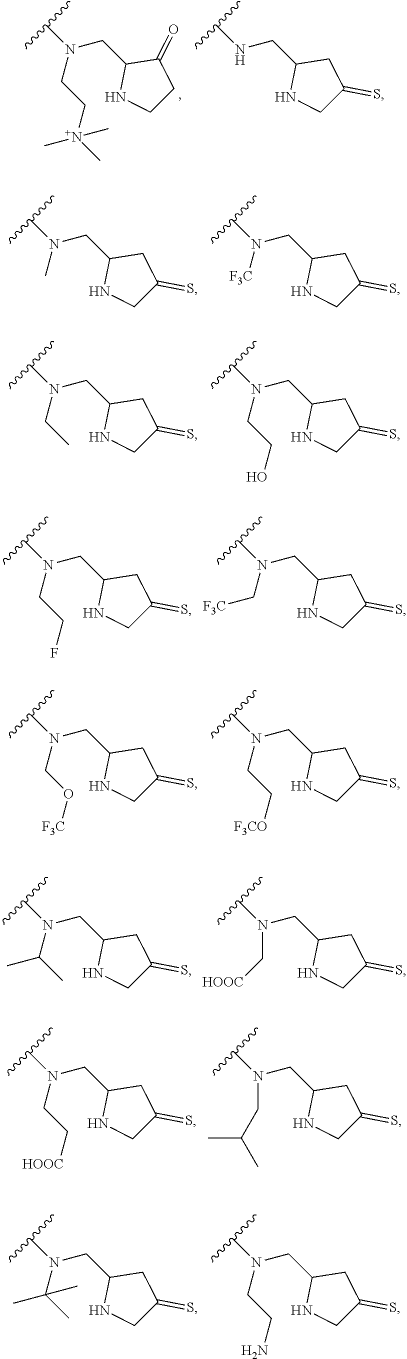

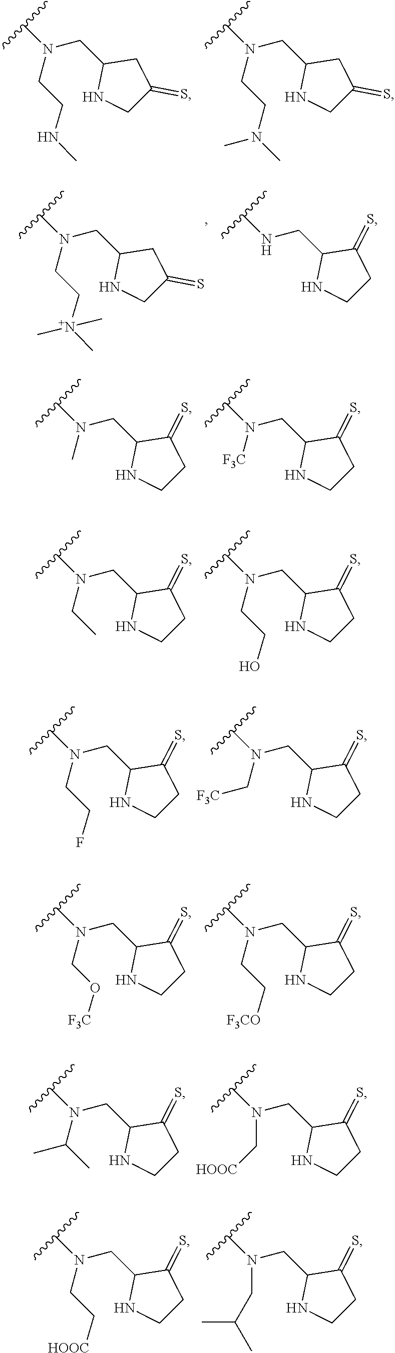

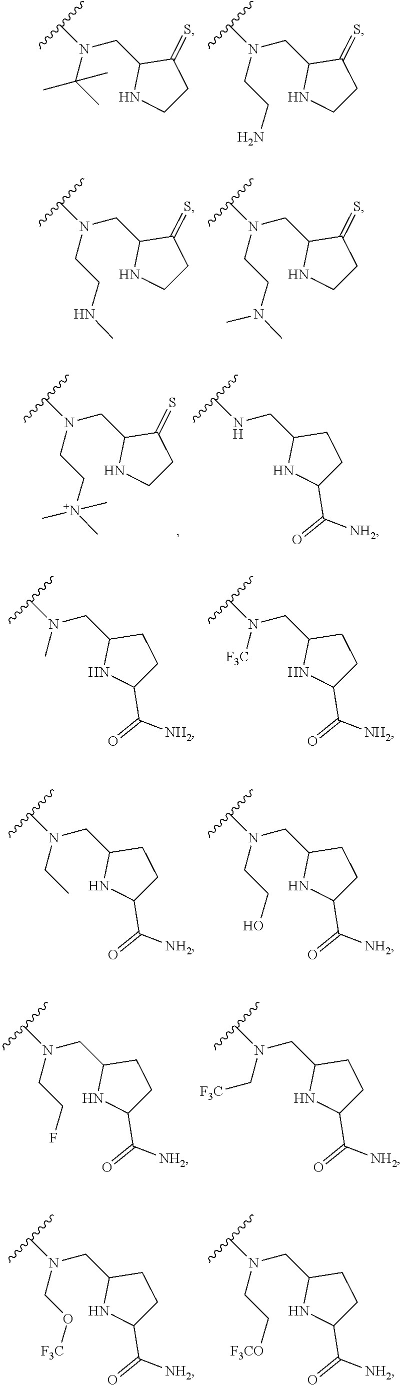

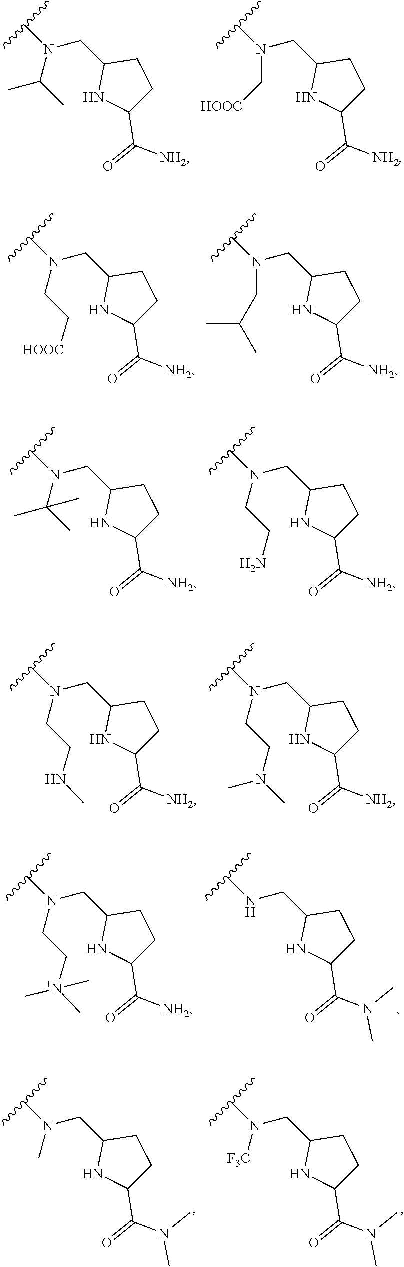

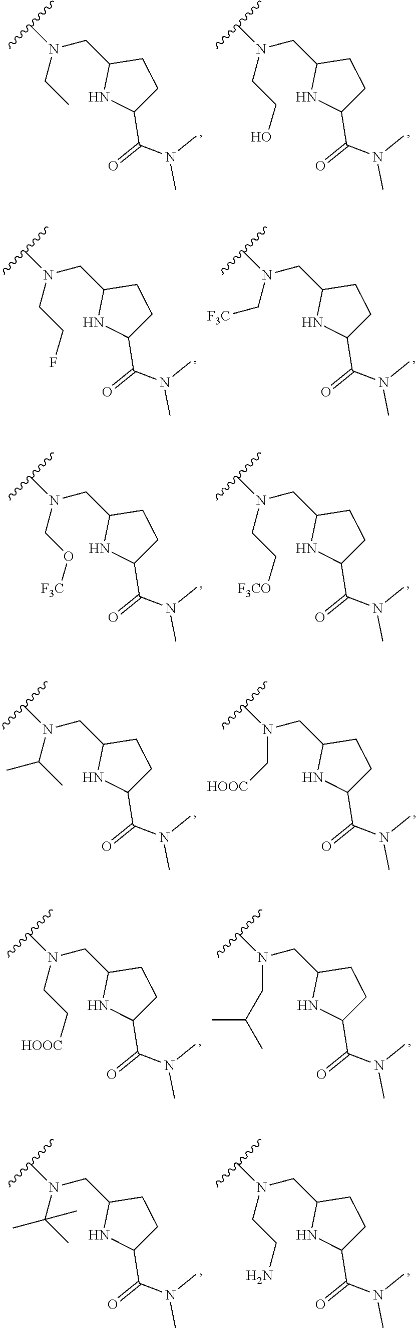

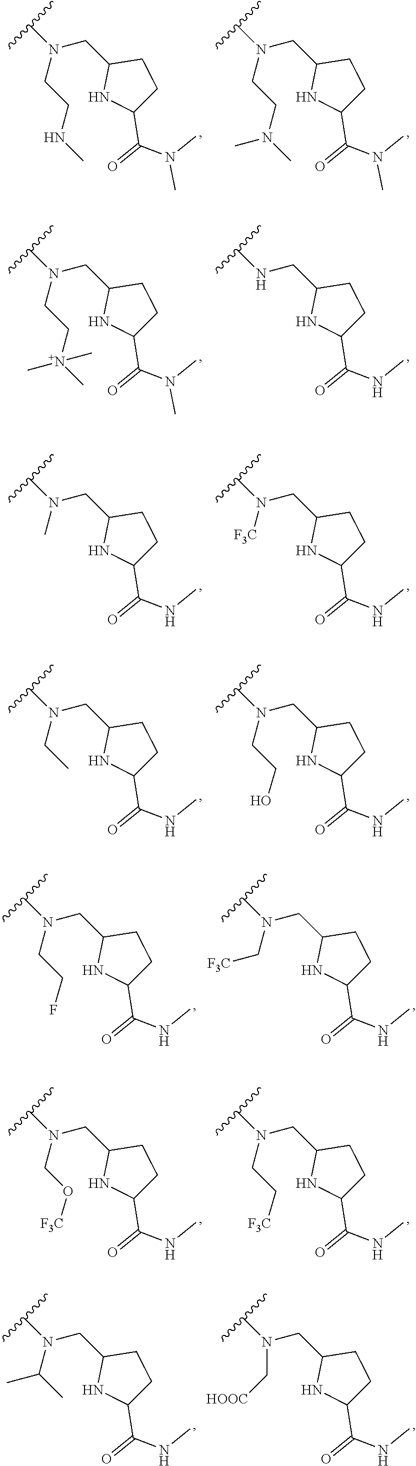

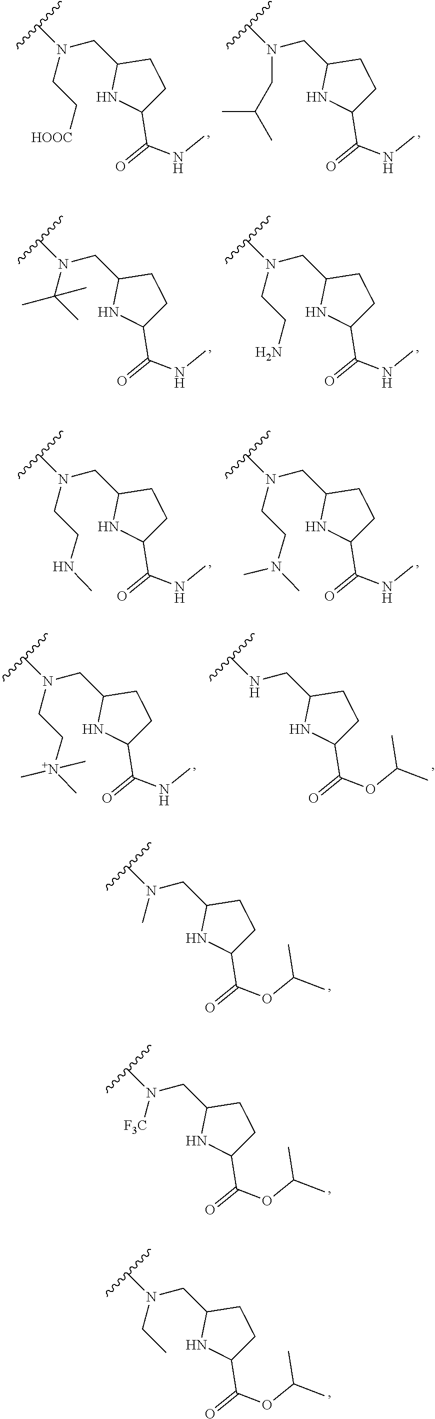

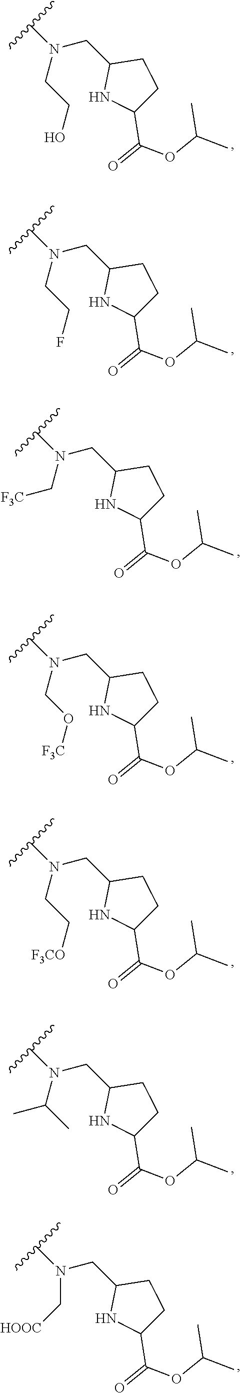

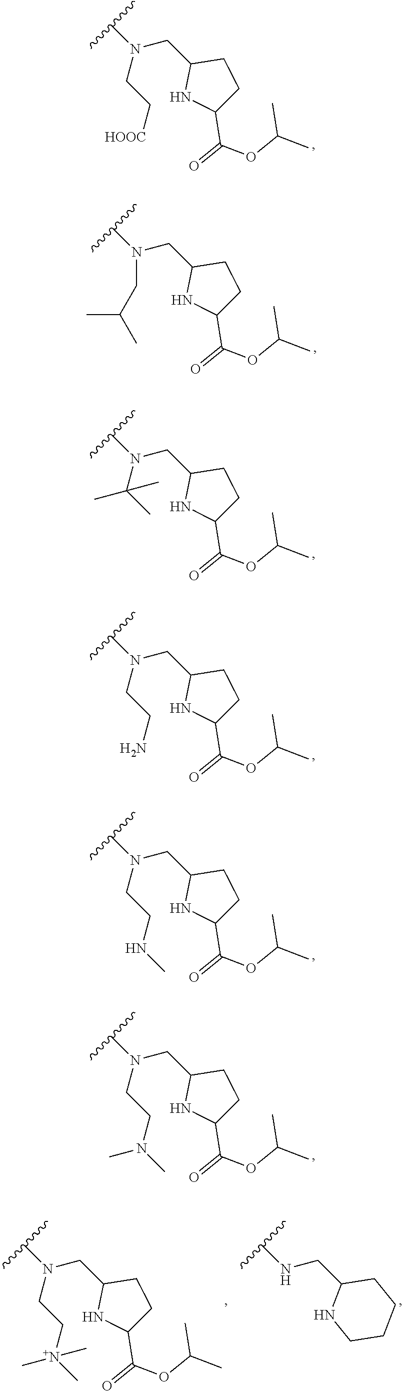

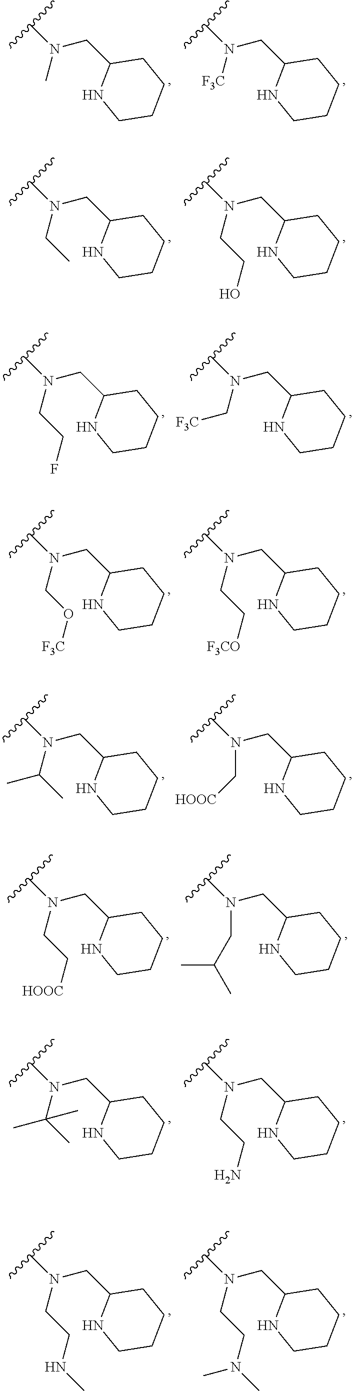

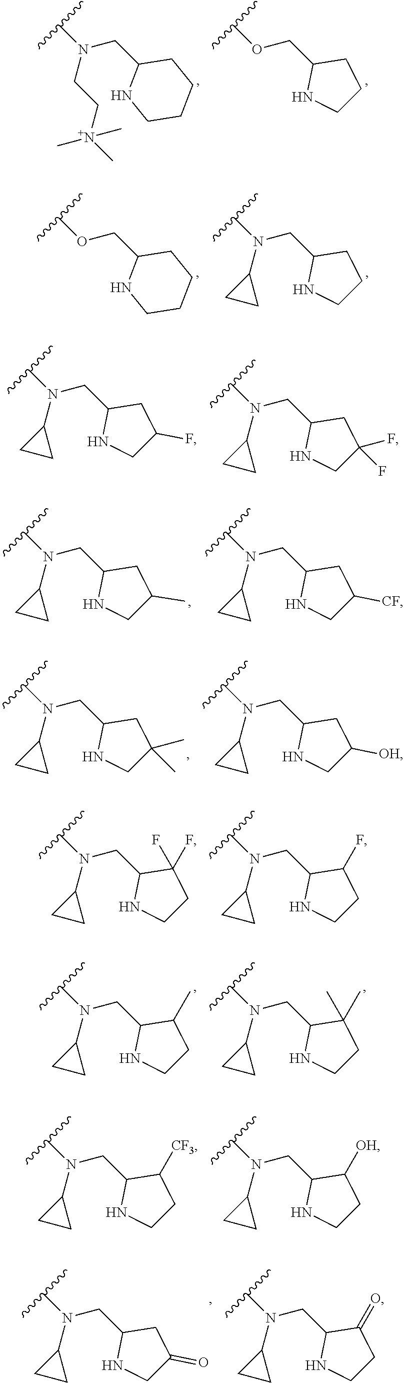

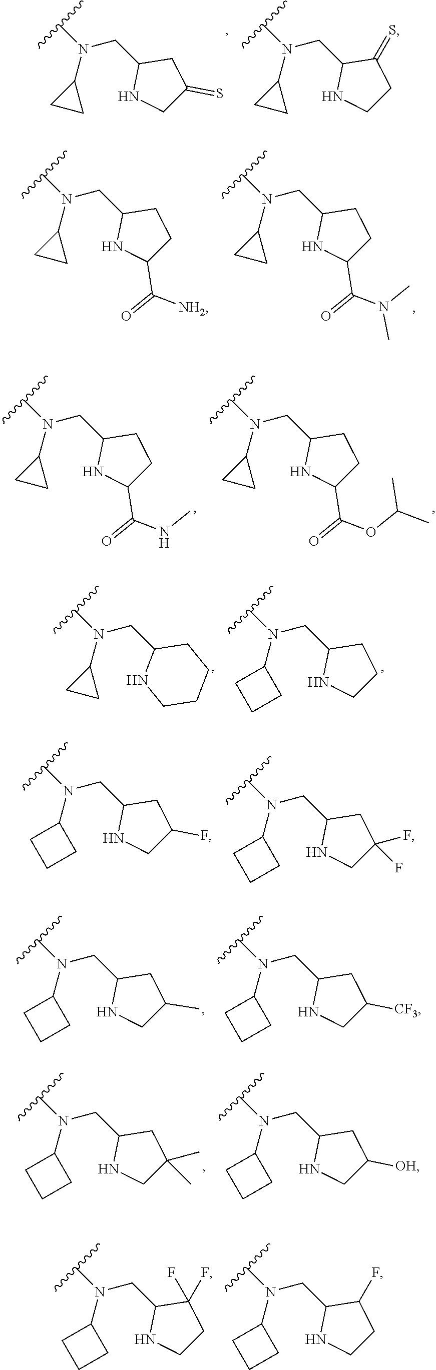

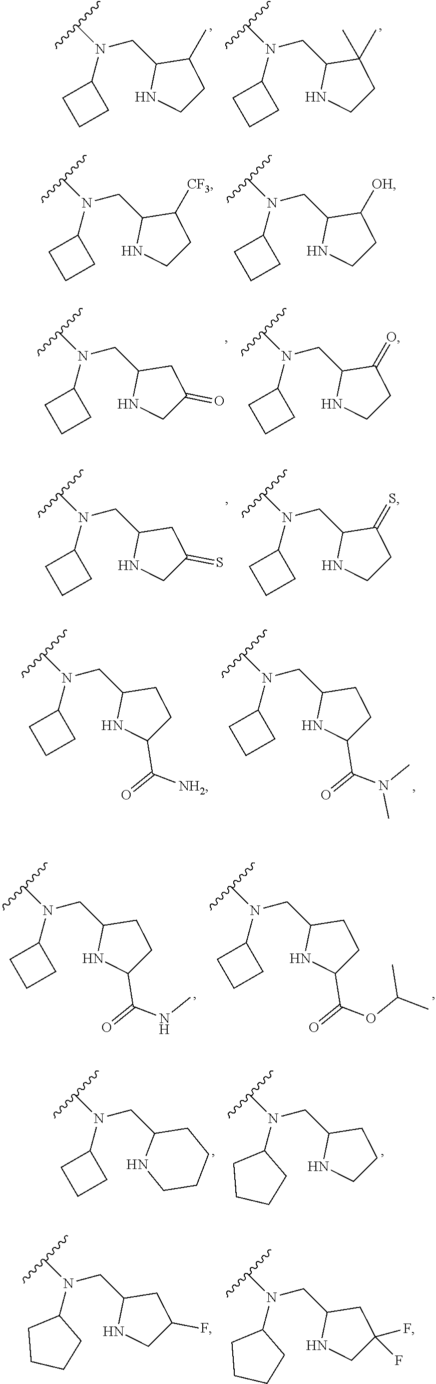

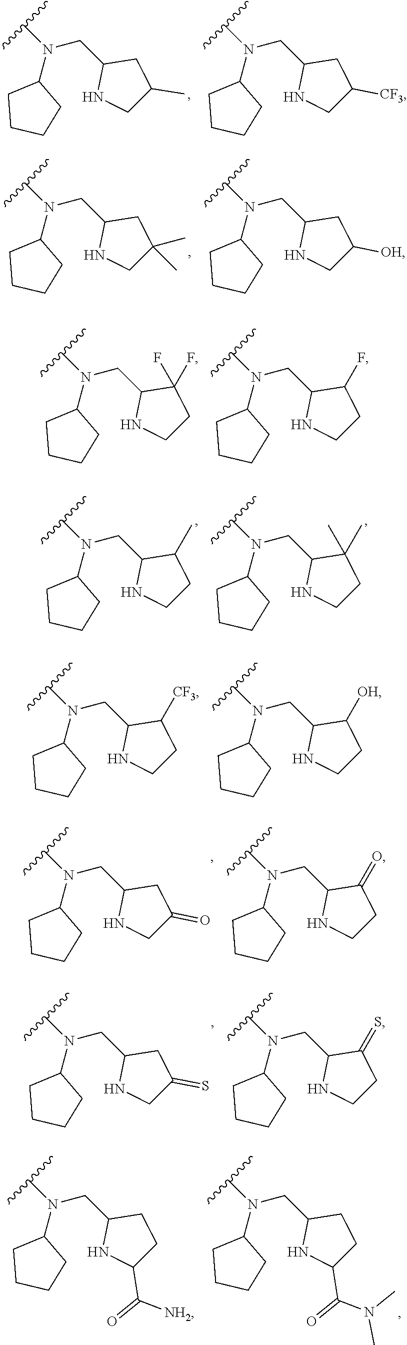

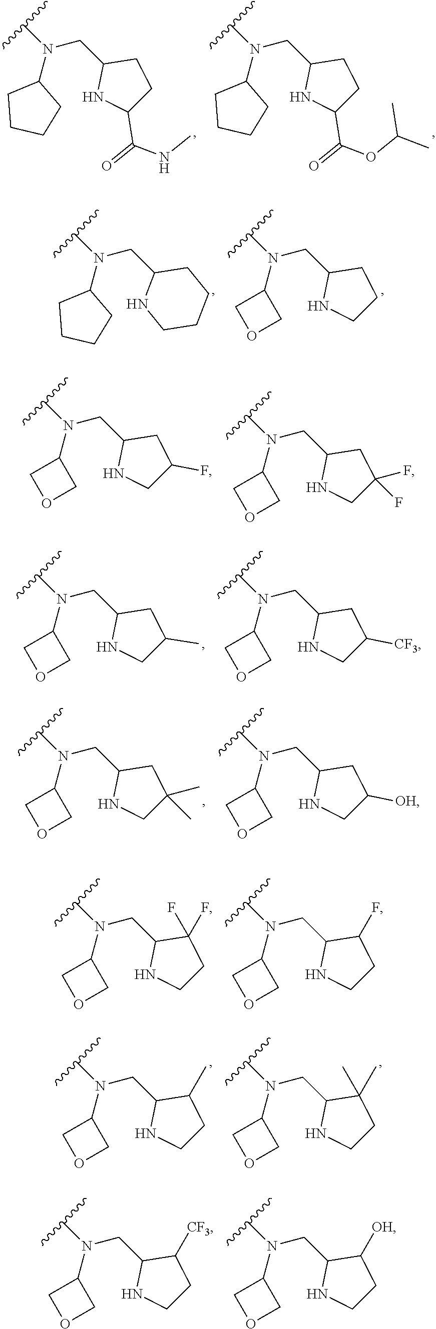

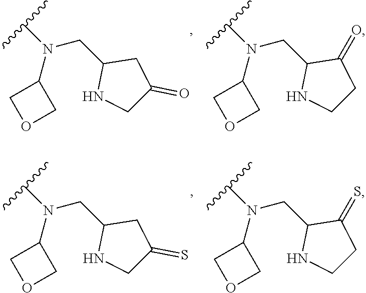

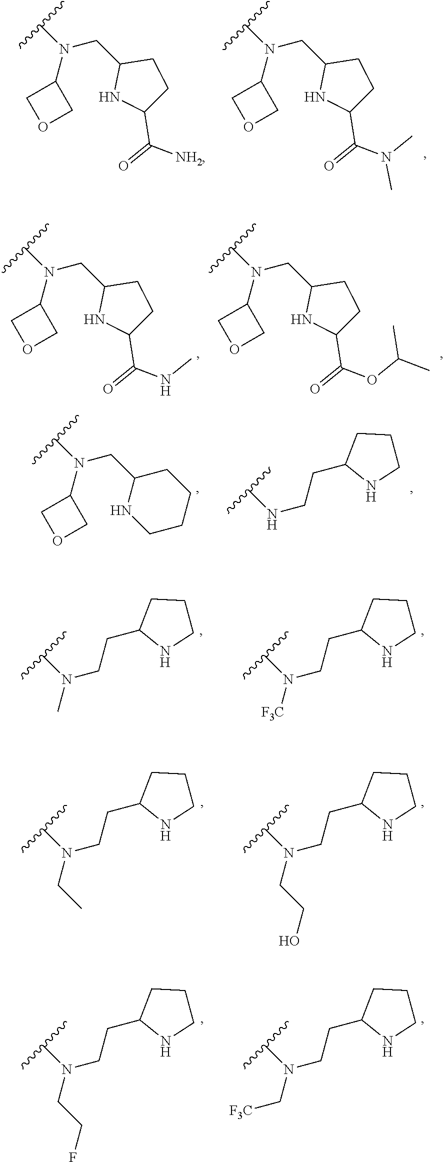

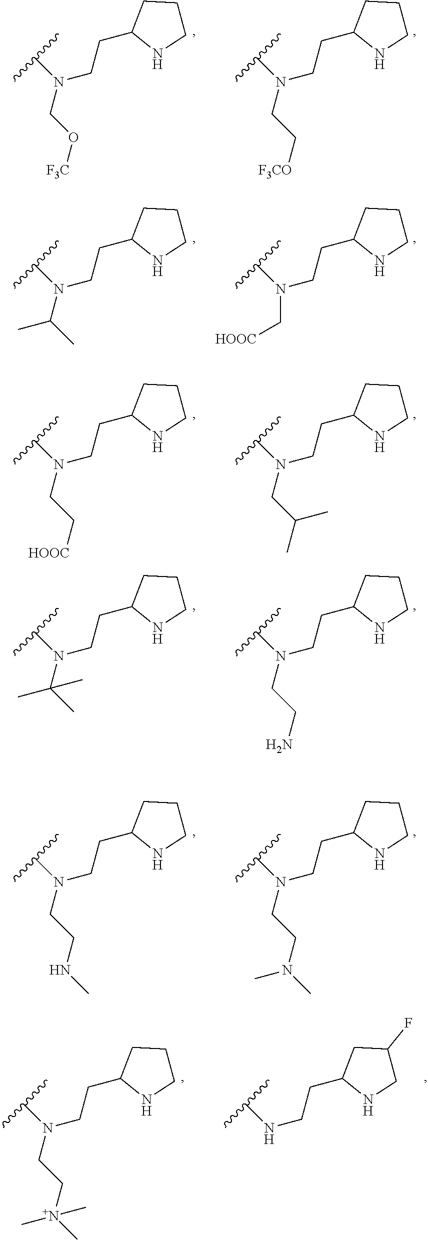

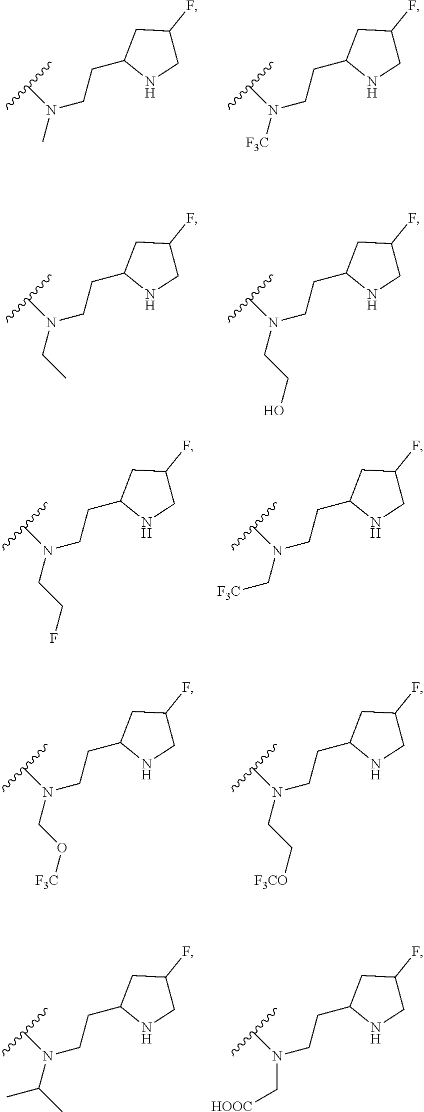

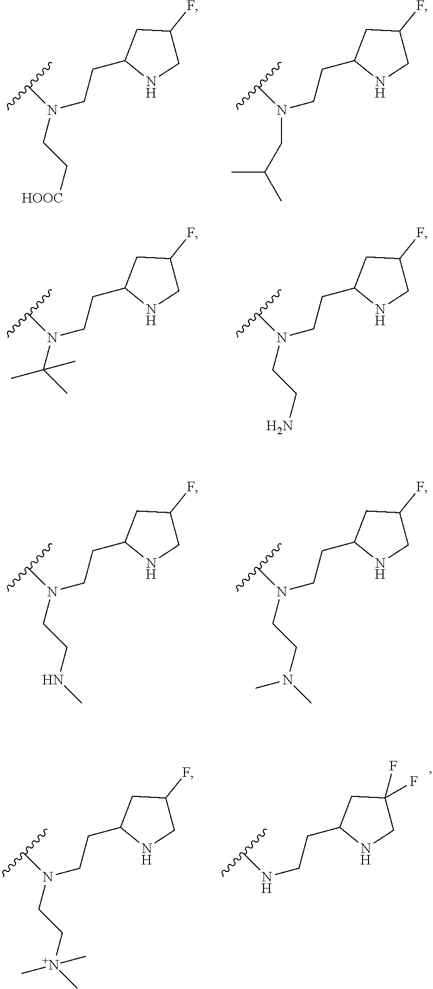

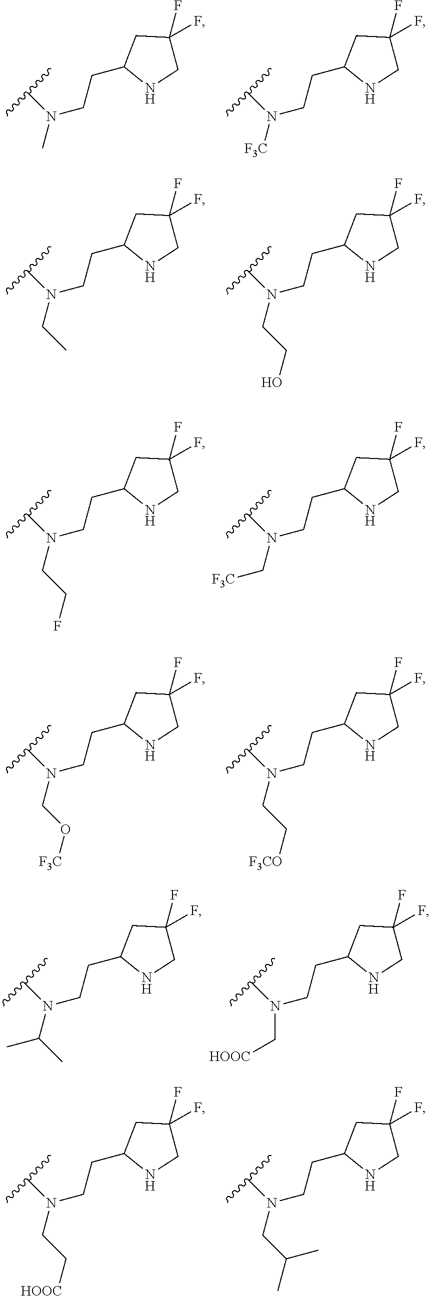

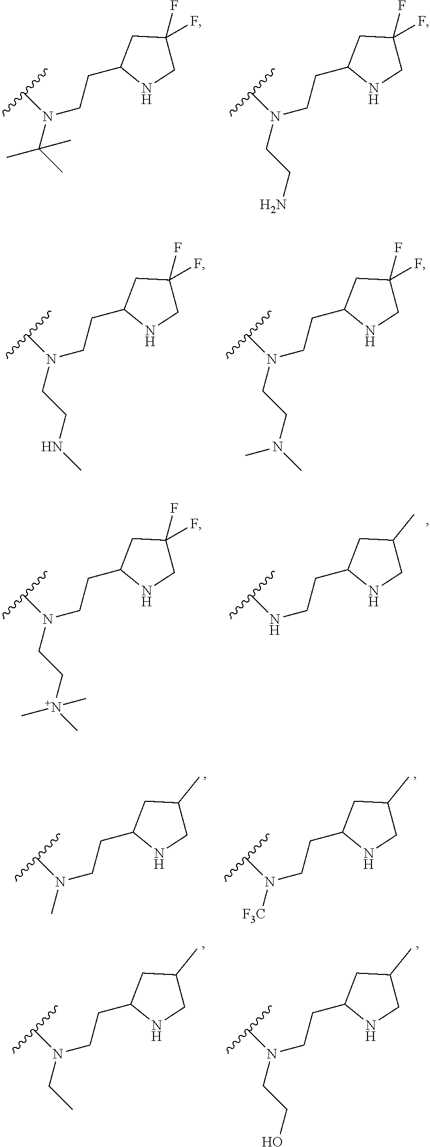

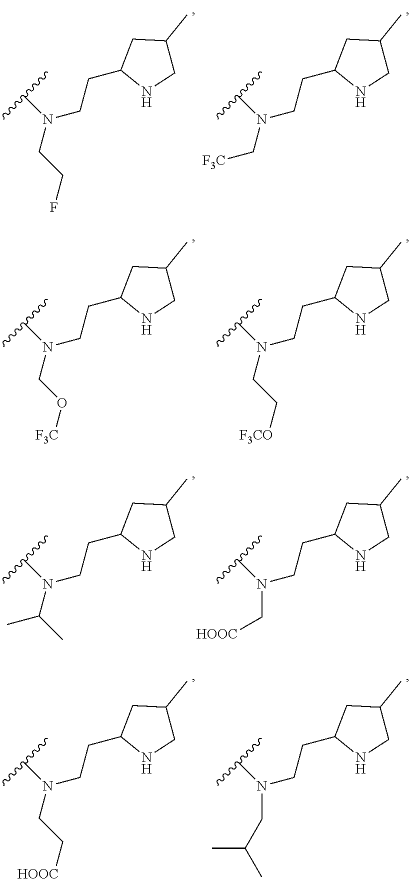

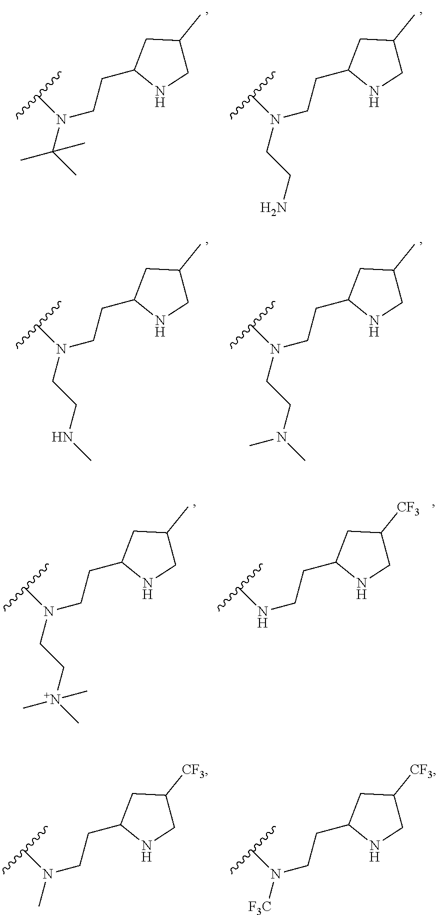

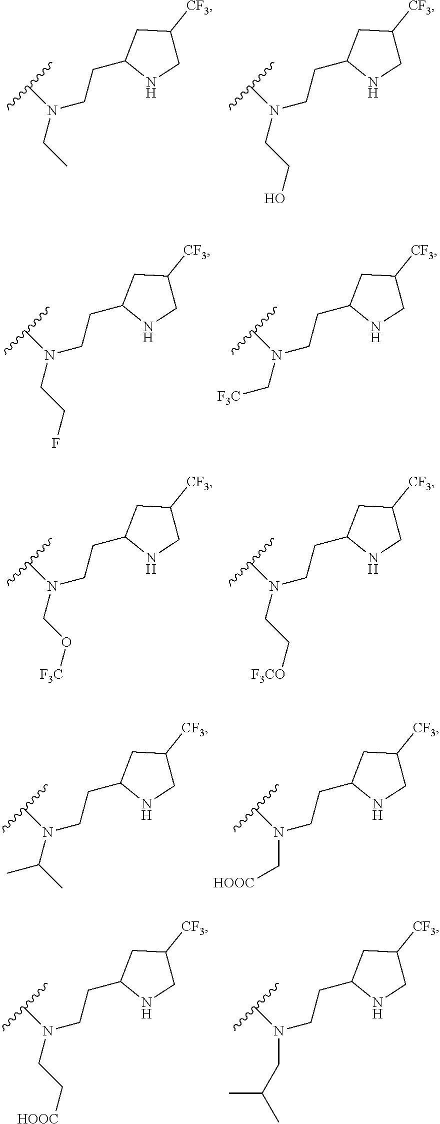

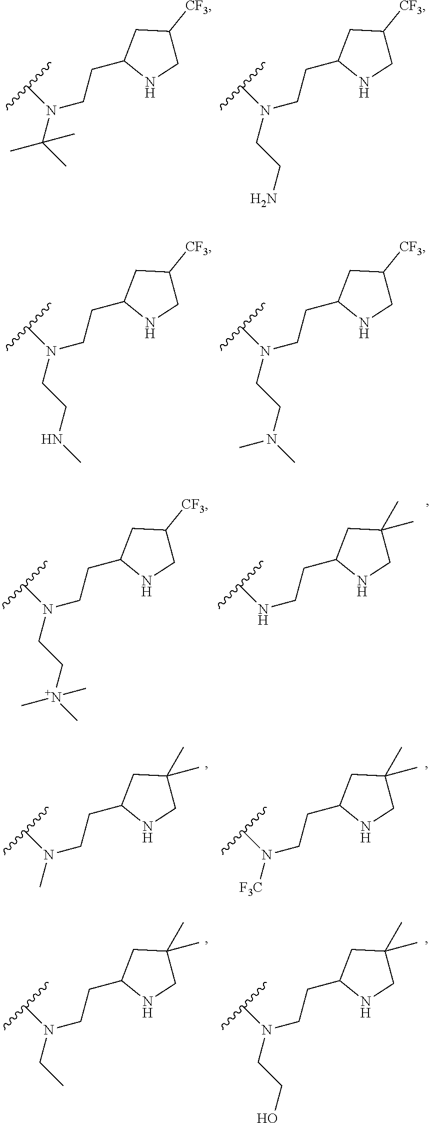

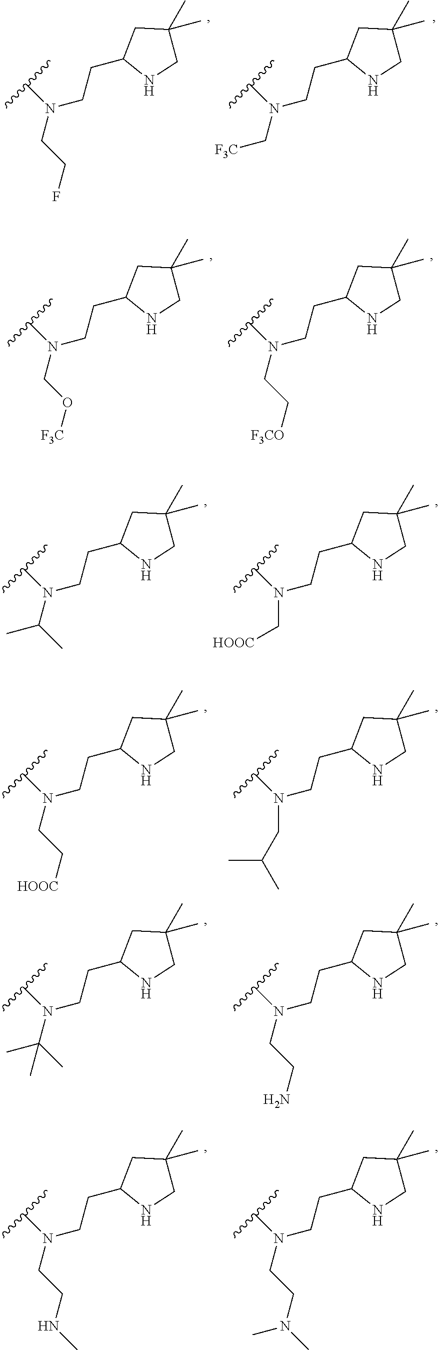

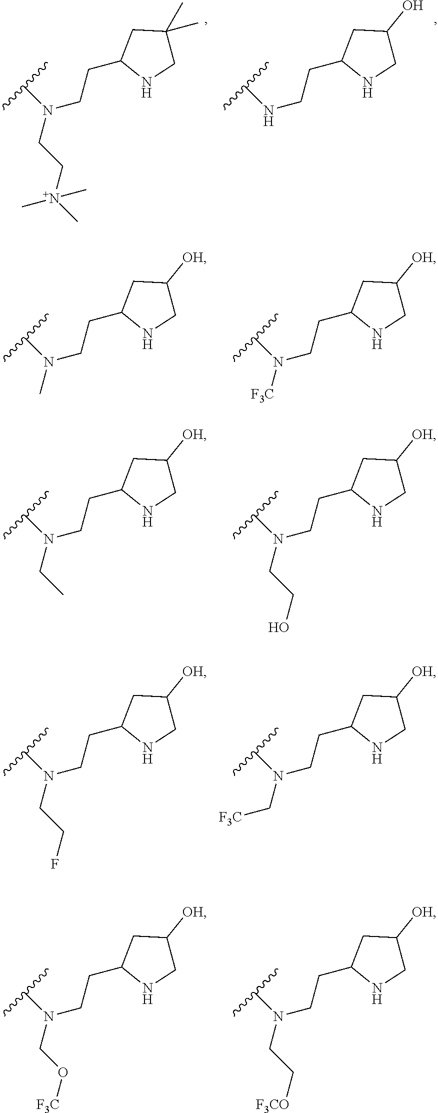

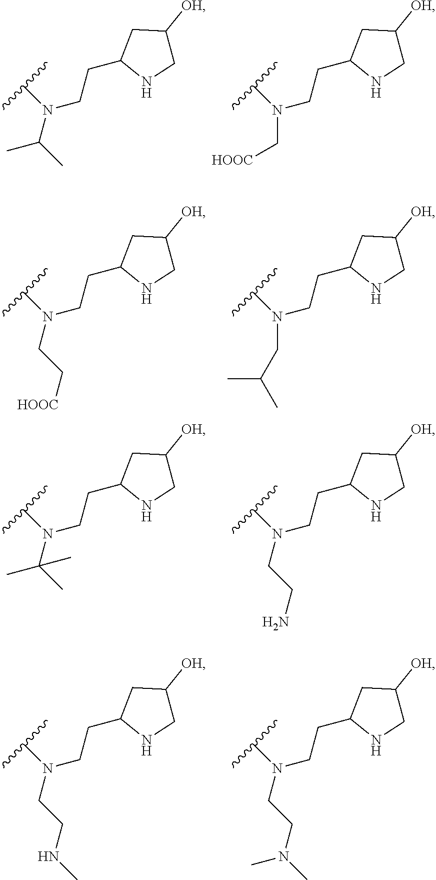

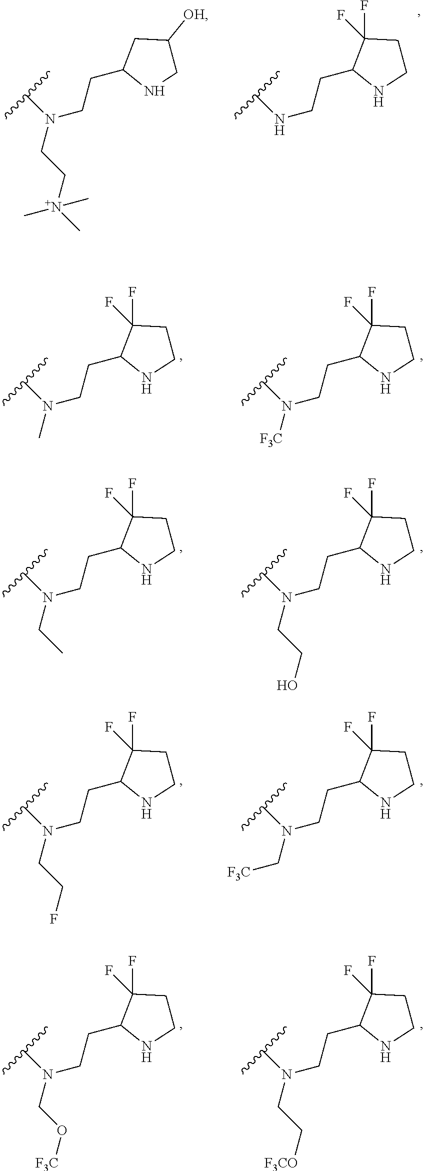

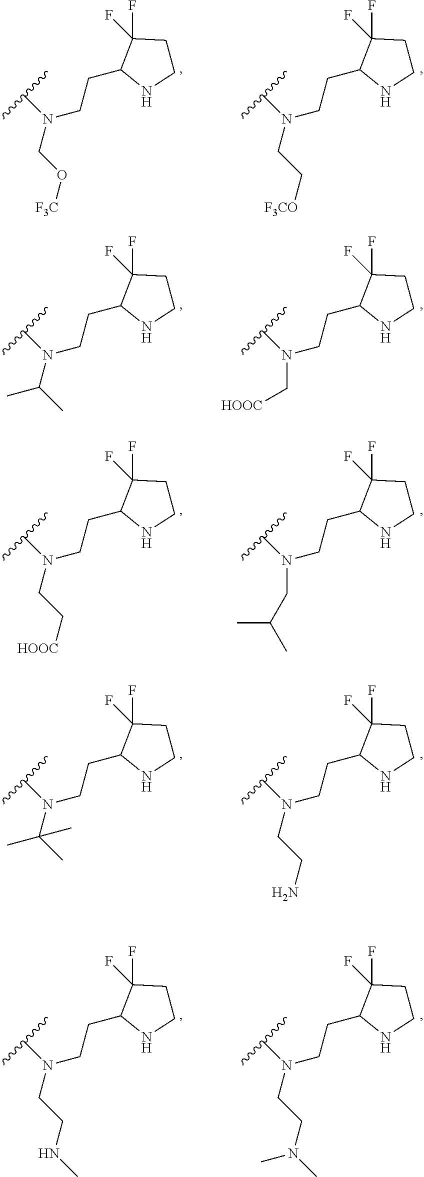

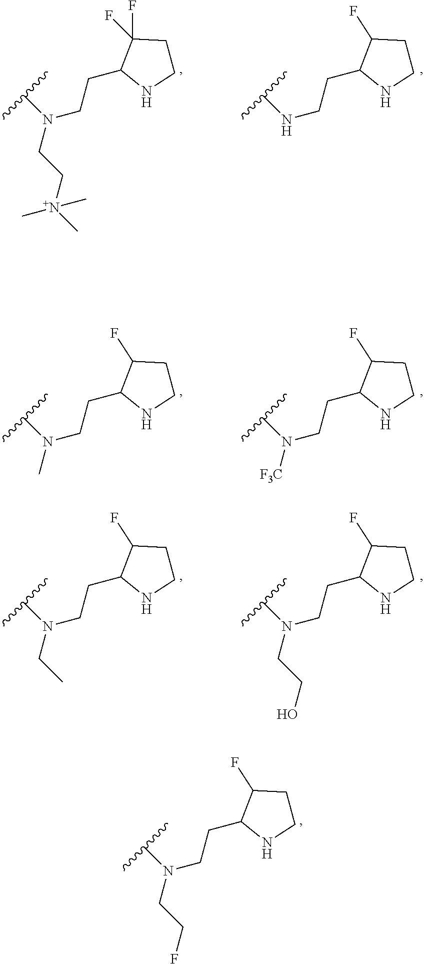

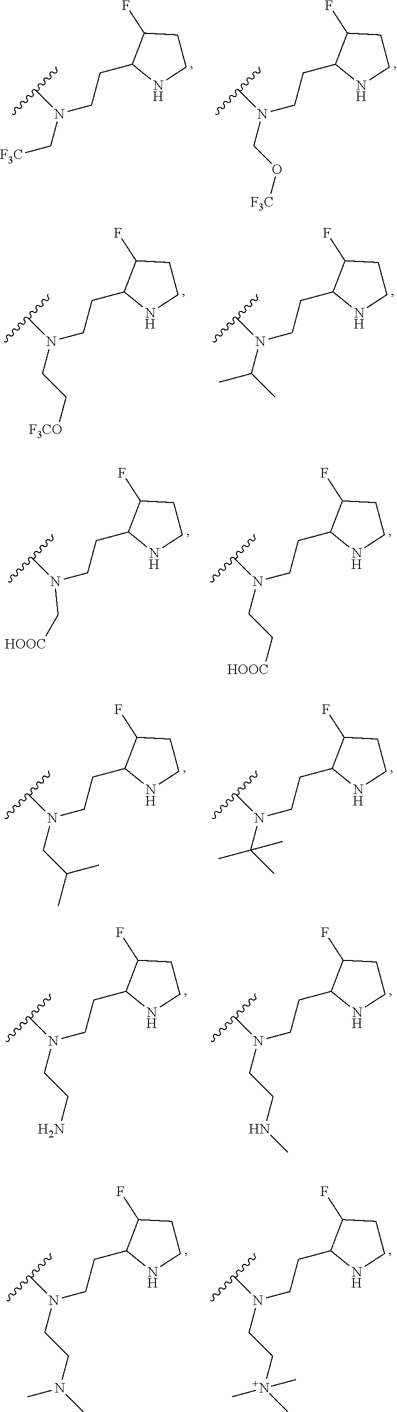

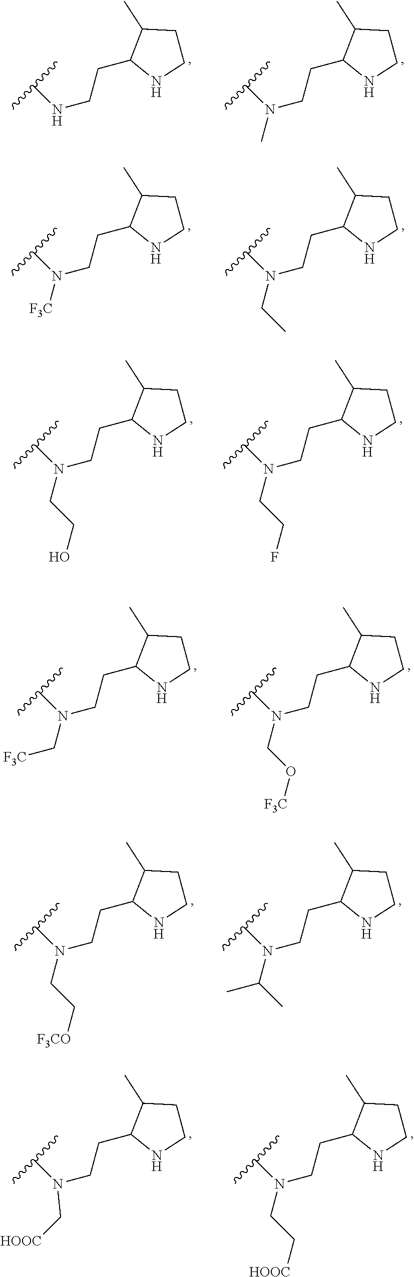

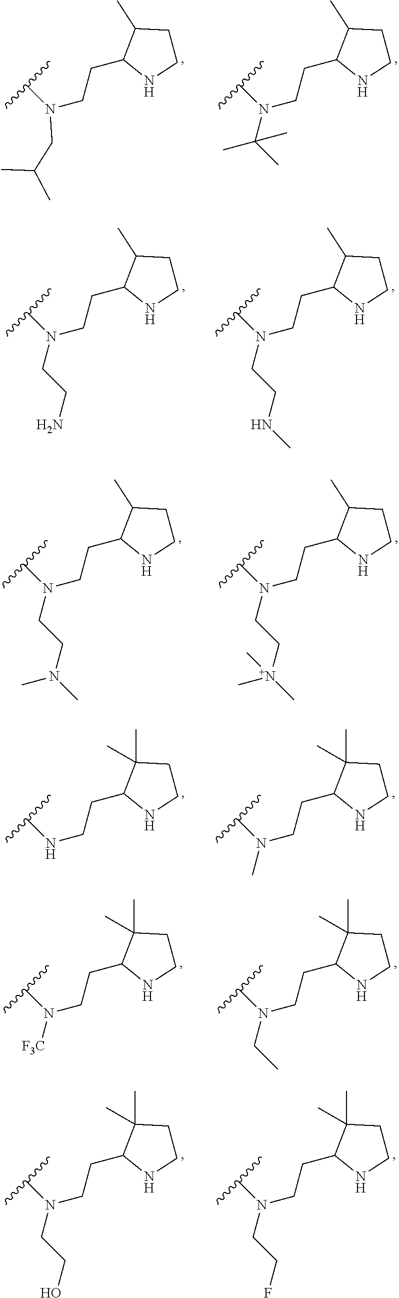

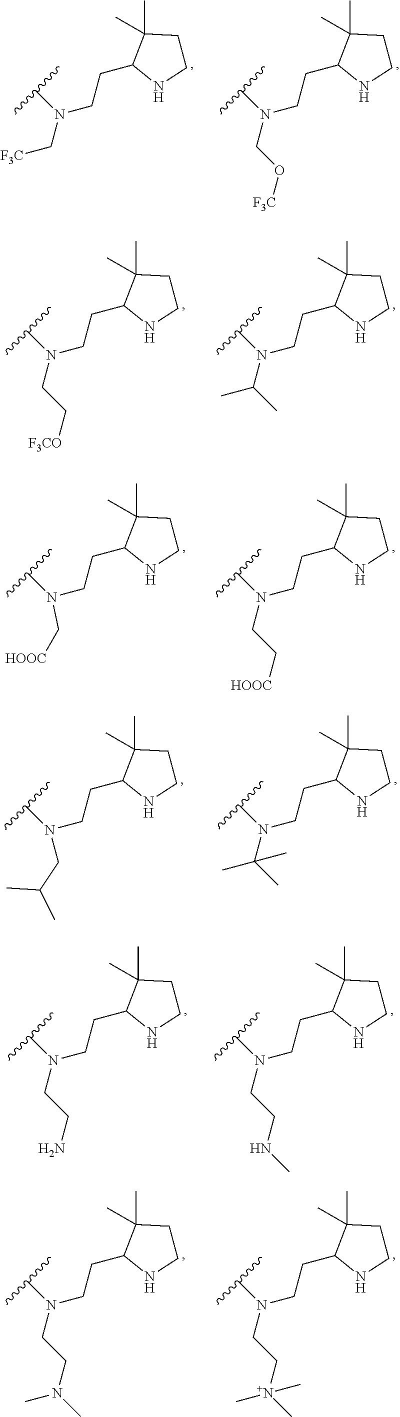

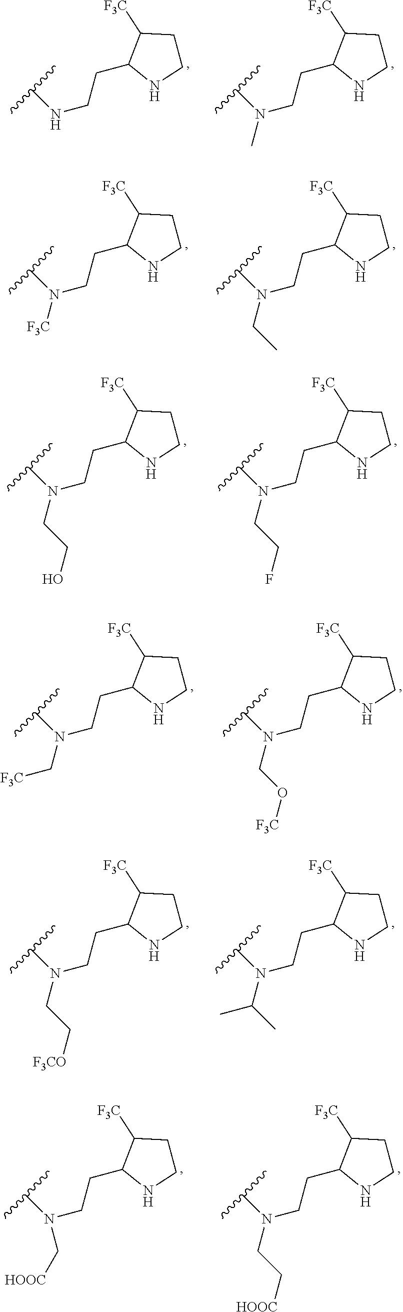

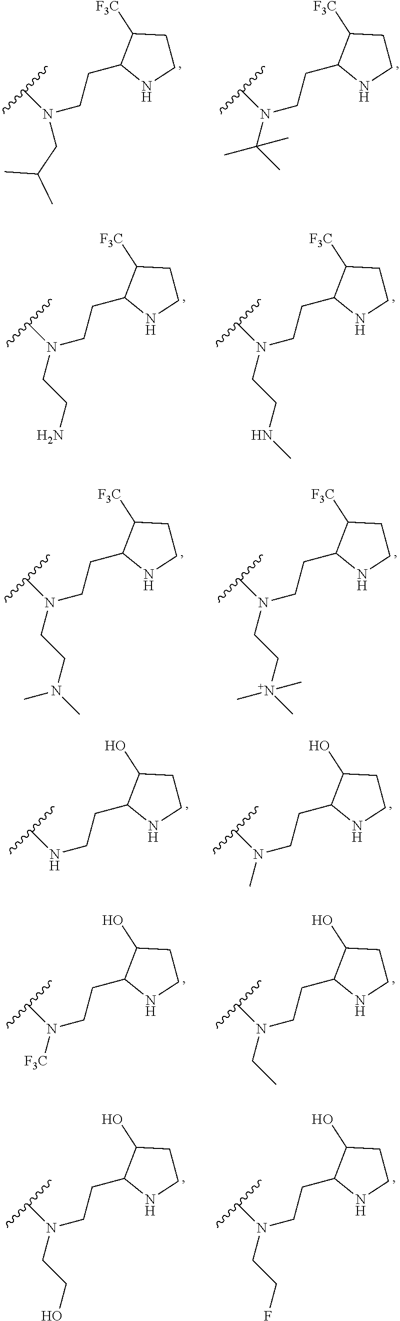

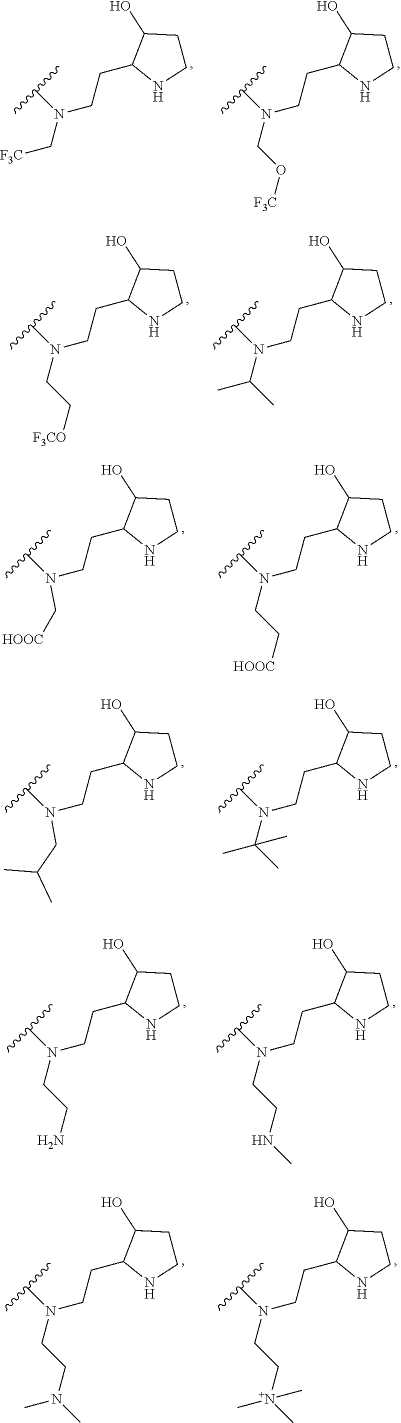

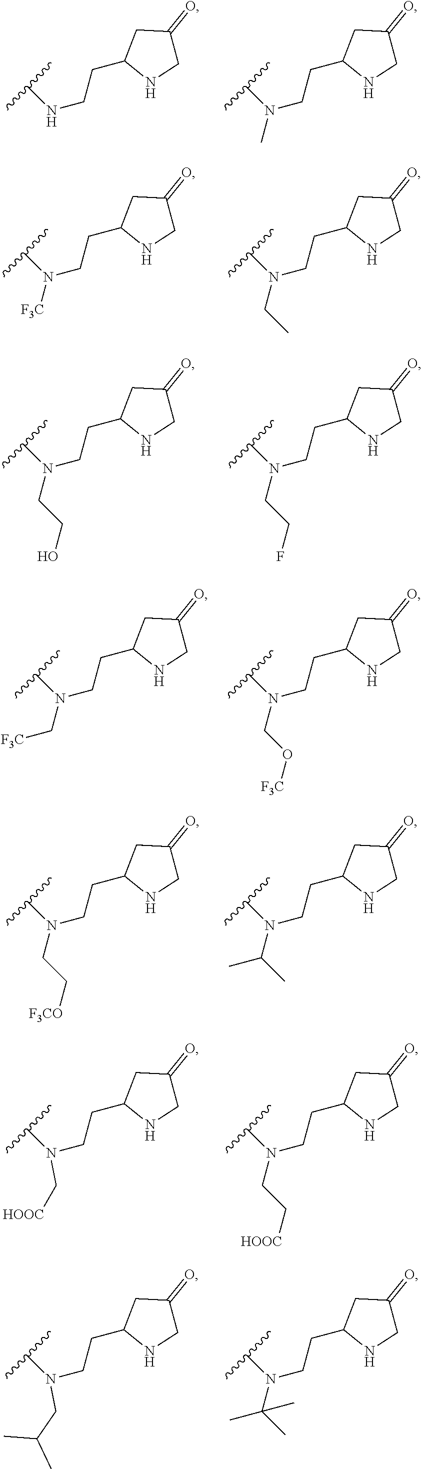

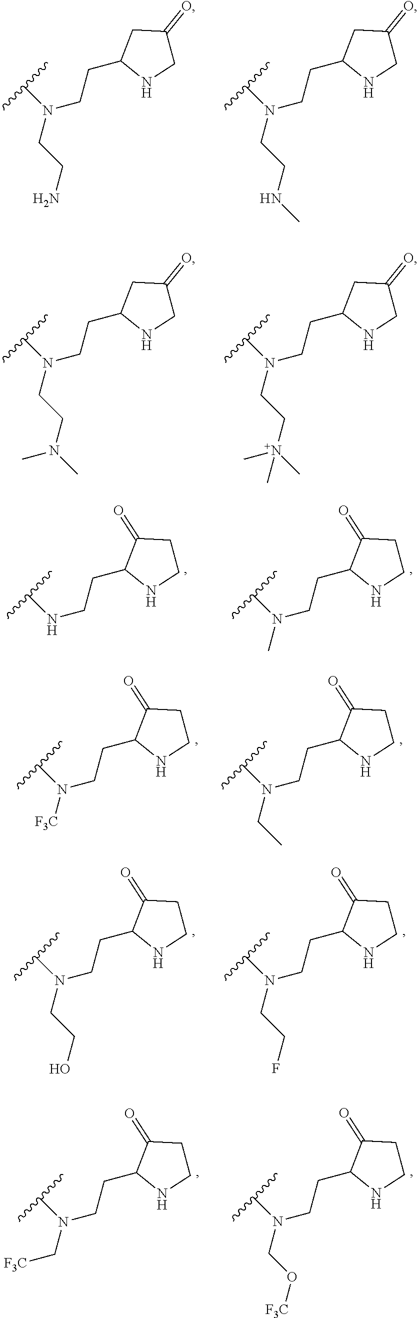

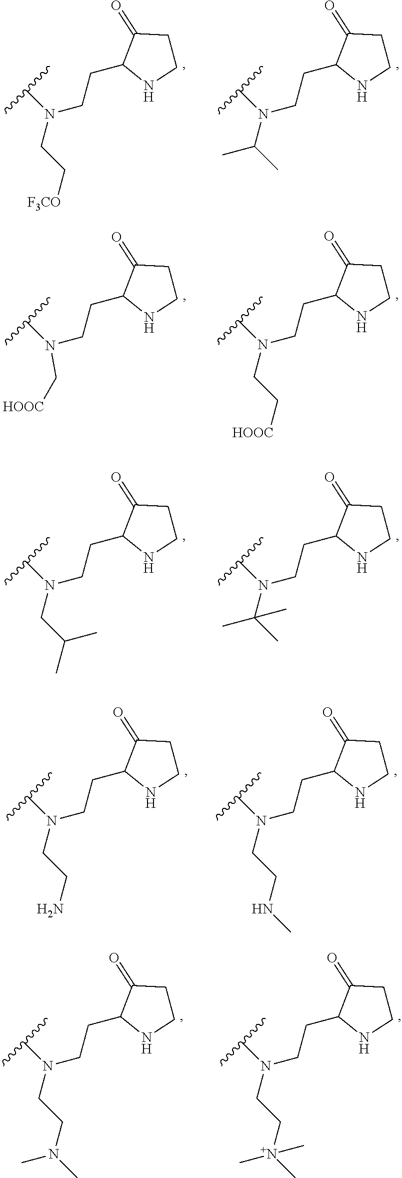

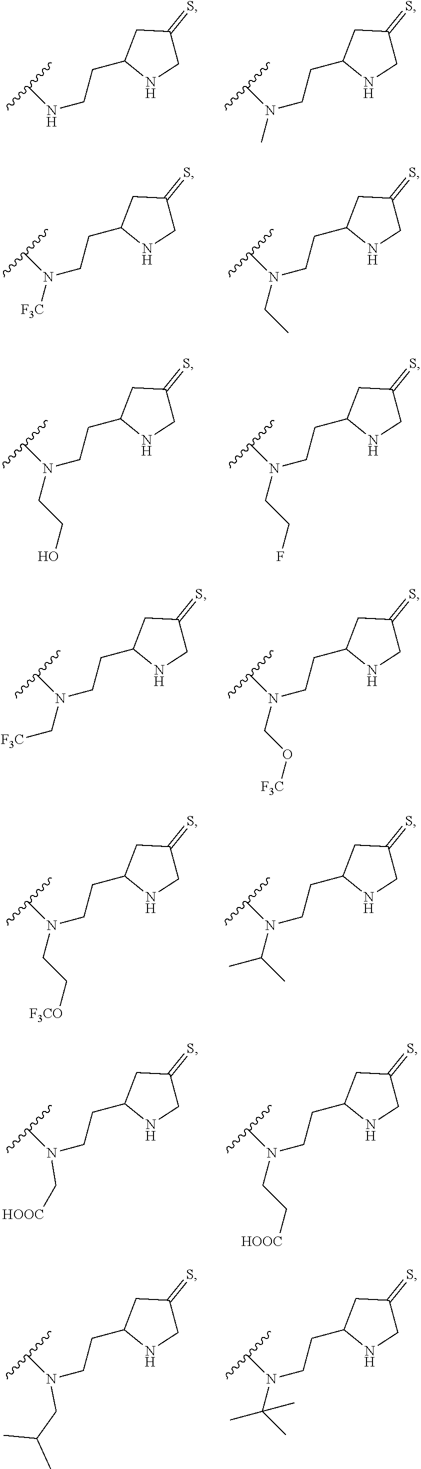

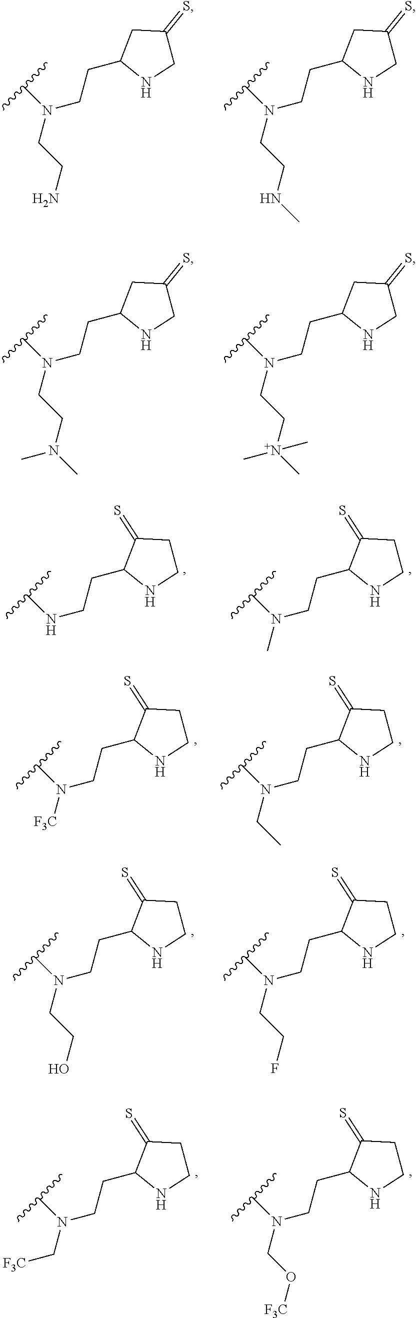

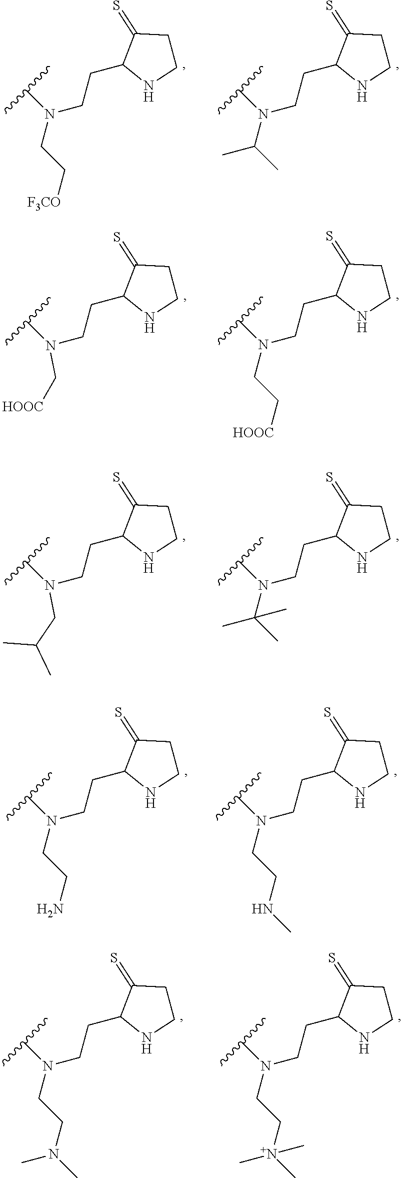

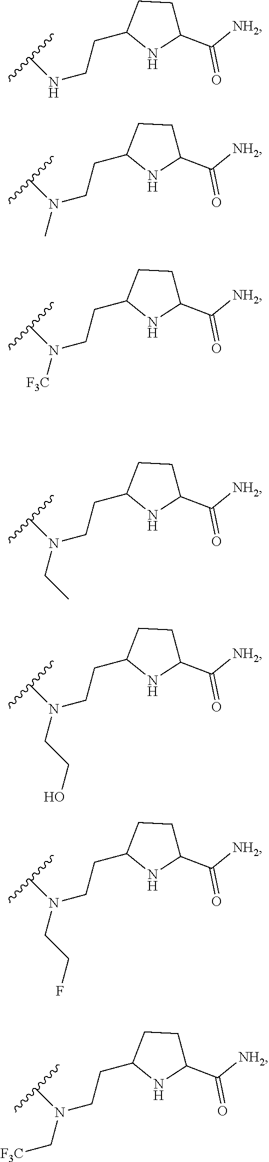

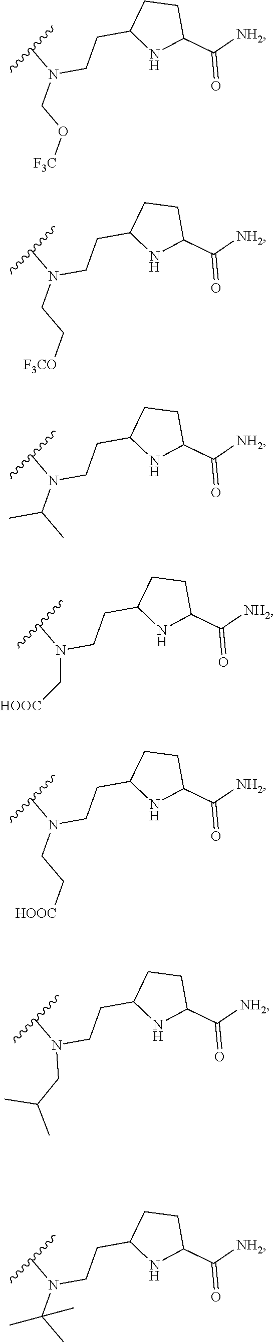

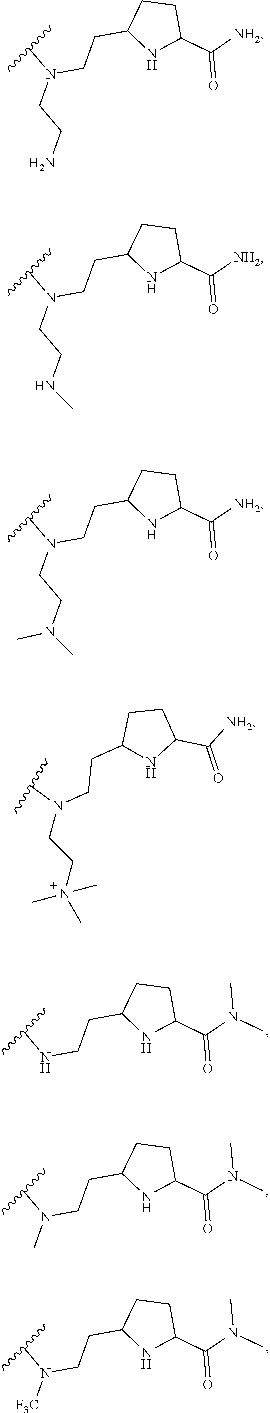

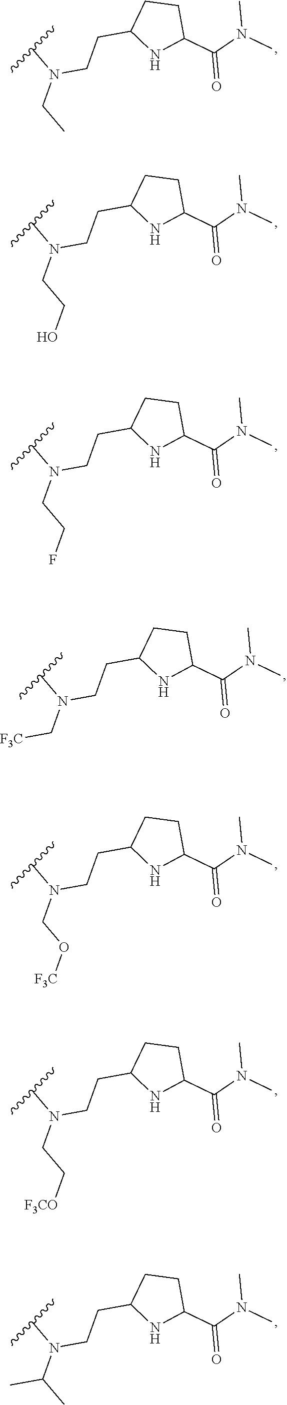

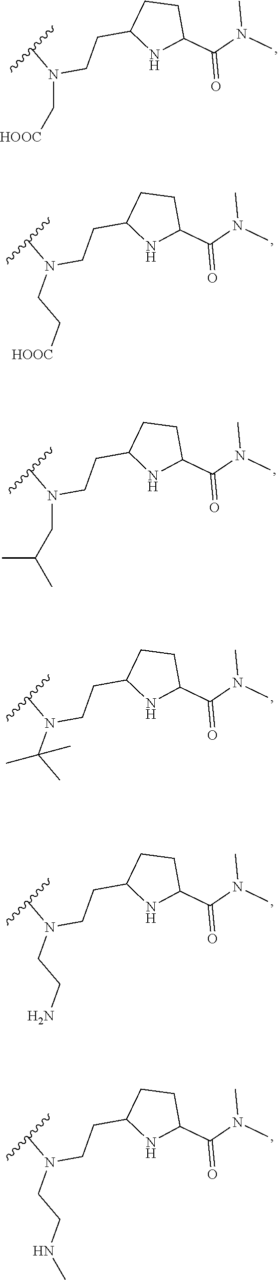

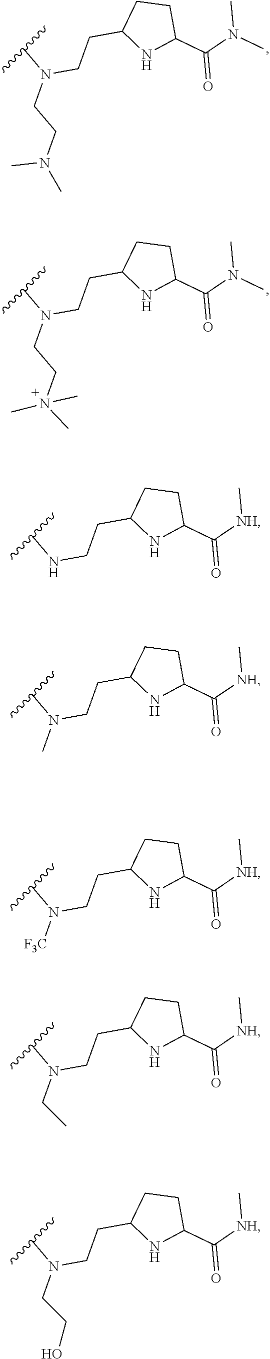

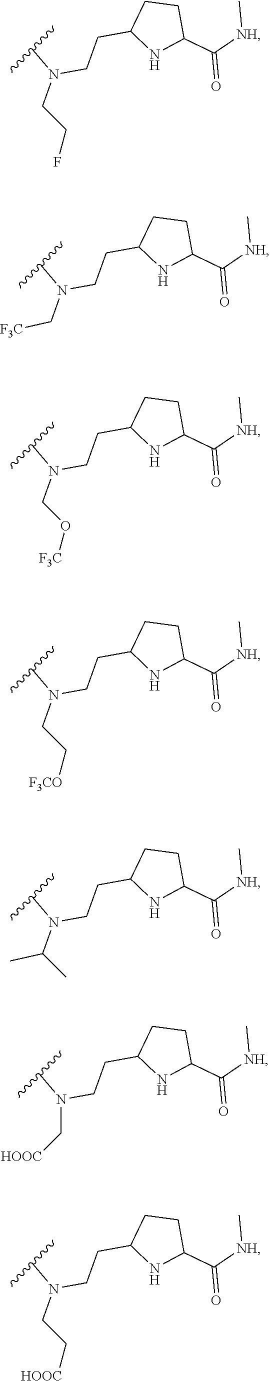

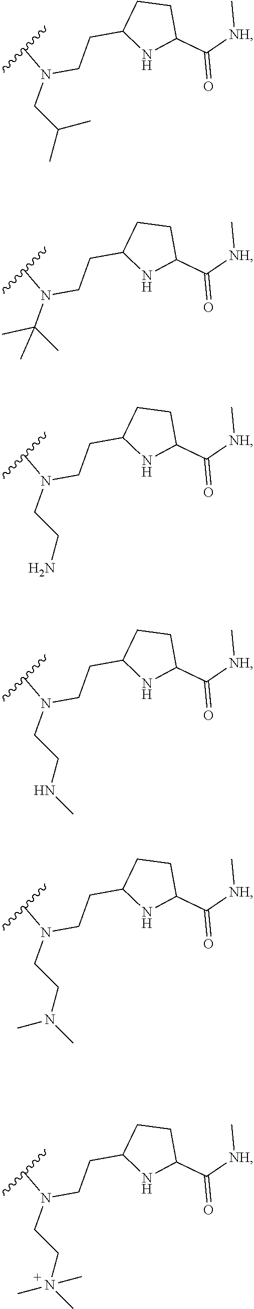

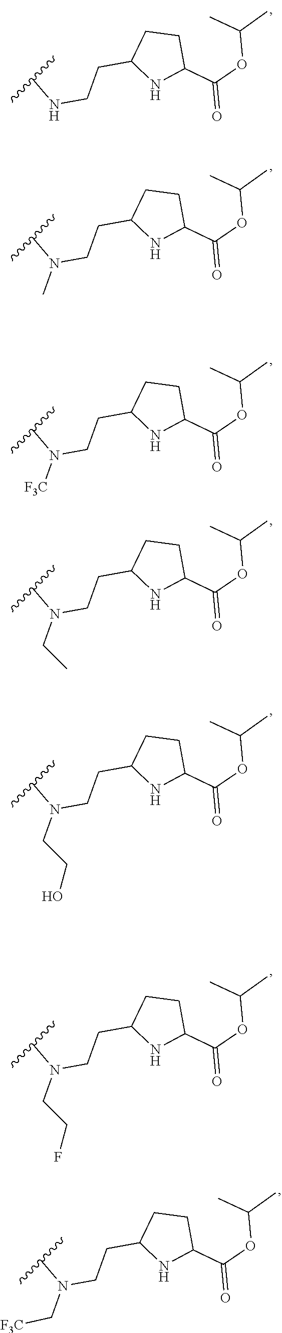

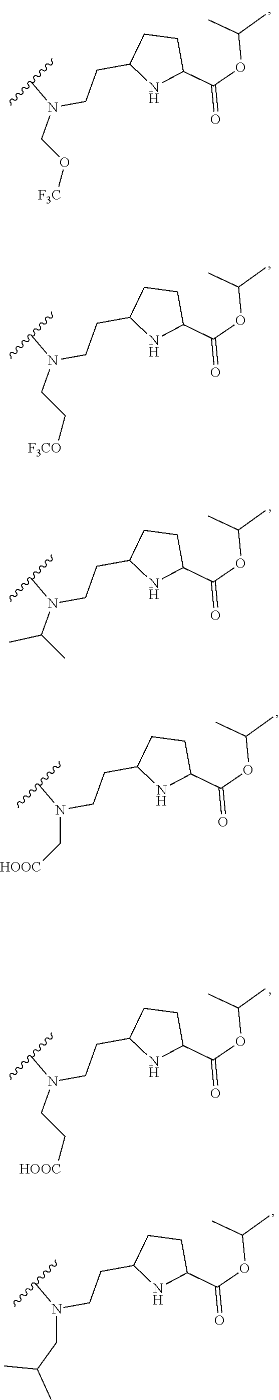

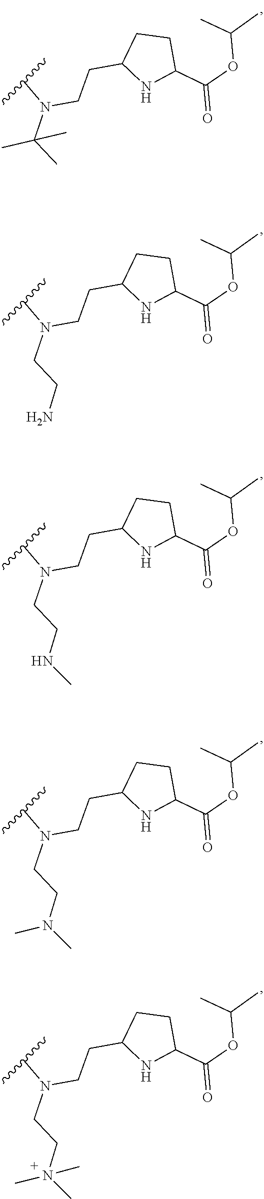

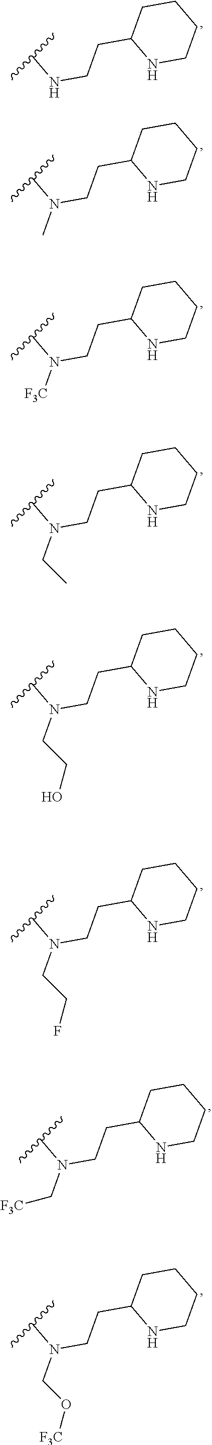

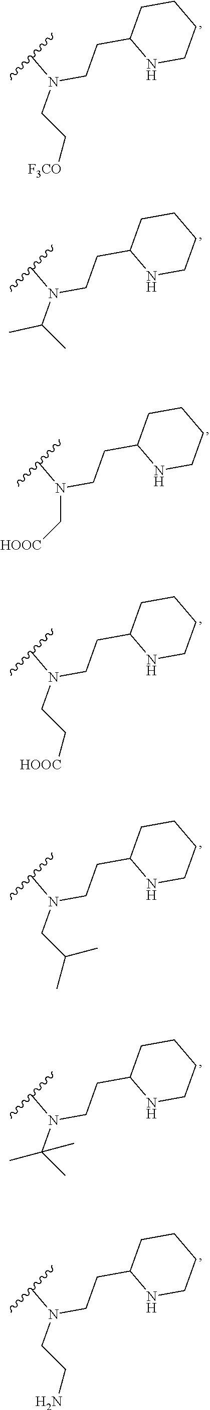

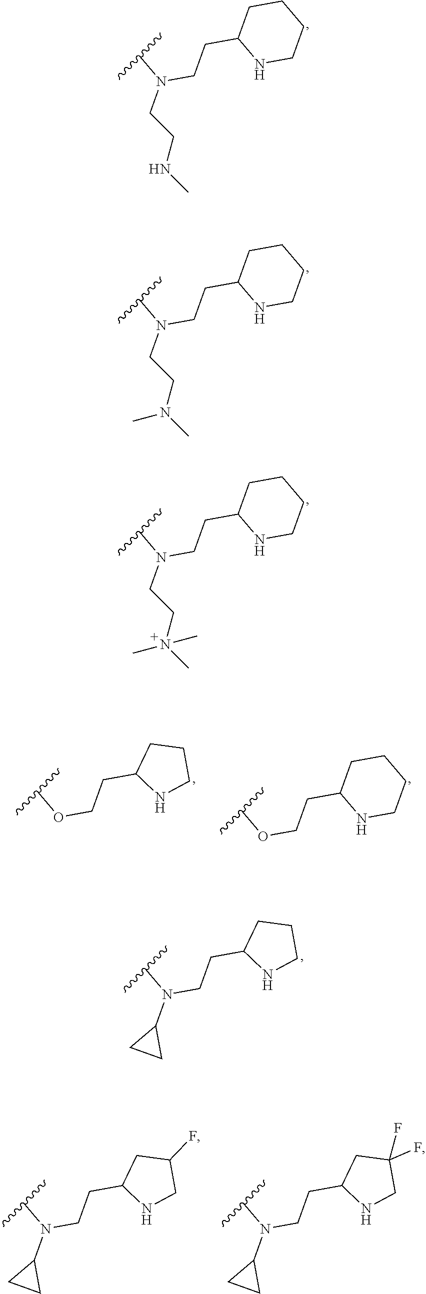

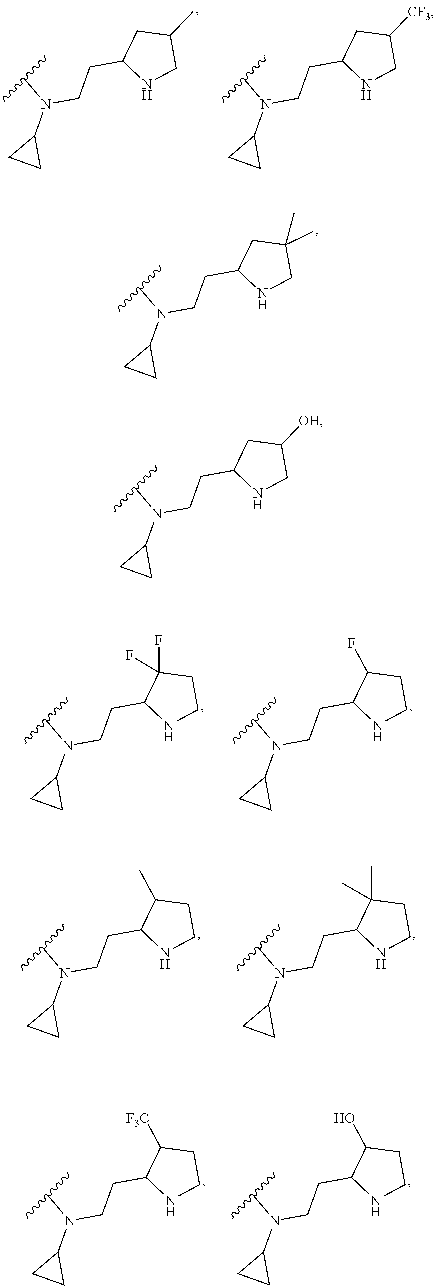

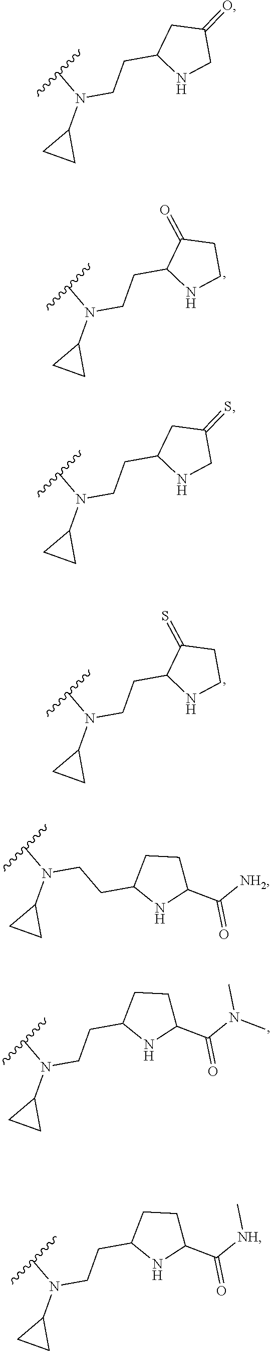

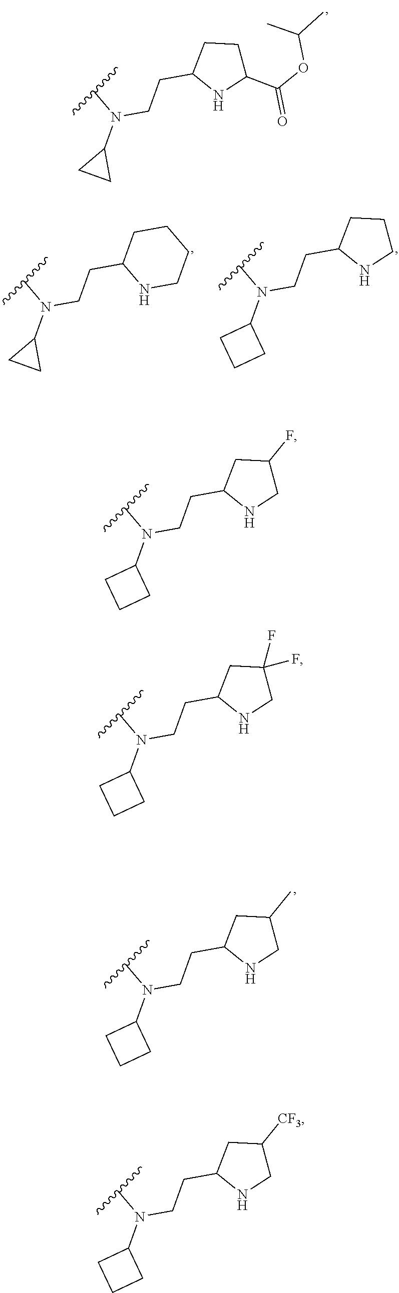

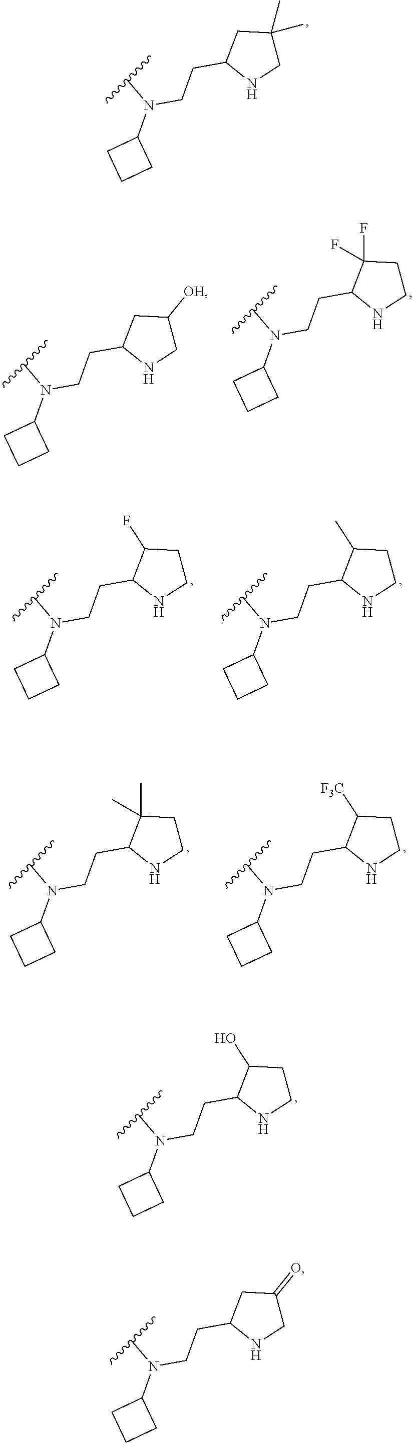

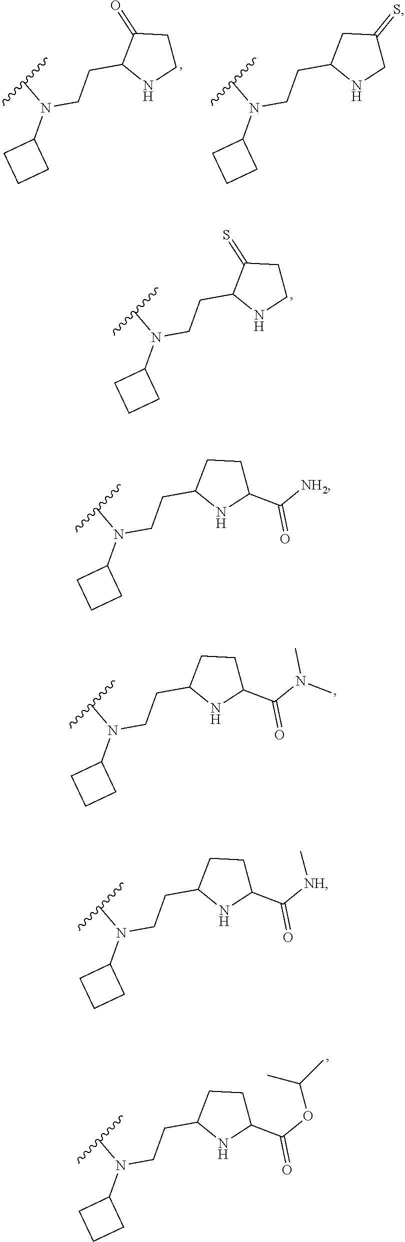

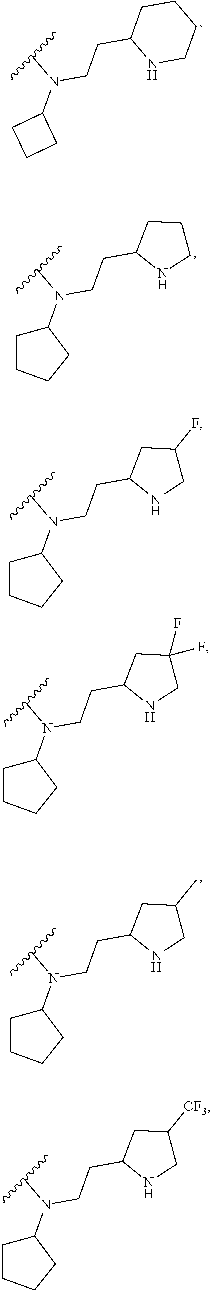 [SMILES: *N(CCC1CCCCN1)C1CCC1 ; *N(CCC1CCCN1)C1CCCC1 ; *N(CCC1CC(F)CN1)C1CCCC1 ; *N(CCC1CC(F)(F)CN1)C1CCCC1 ; *N(CCC1CC(C)CN1)C1CCCC1 ; *N(CCC1CC(C(F)(F)F)CN1)C1CCCC1]

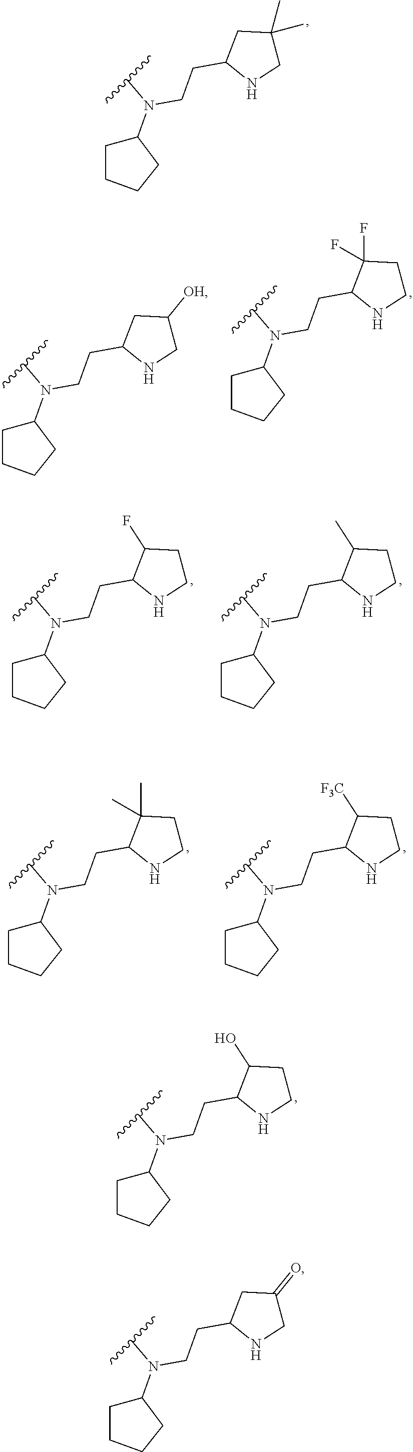

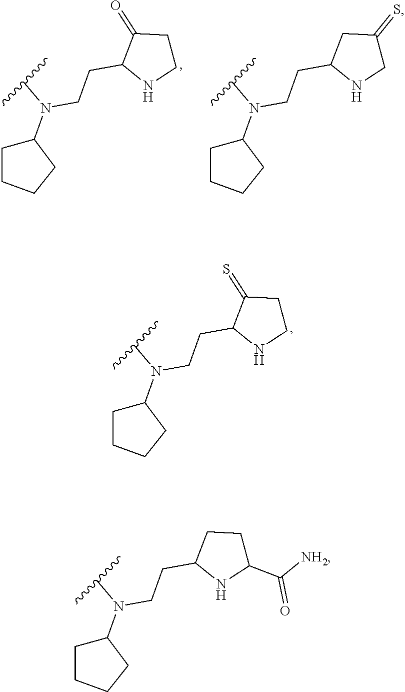 [SMILES: *N(CCC1NCCC1=O)C1CCCC1 ; *N(CCC1CC(=S)CN1)C1CCCC1 ; *N(CCC1NCCC1=S)C1CCCC1 ; *N(CCC1CCC(C(N)=O)N1)C1CCCC1]

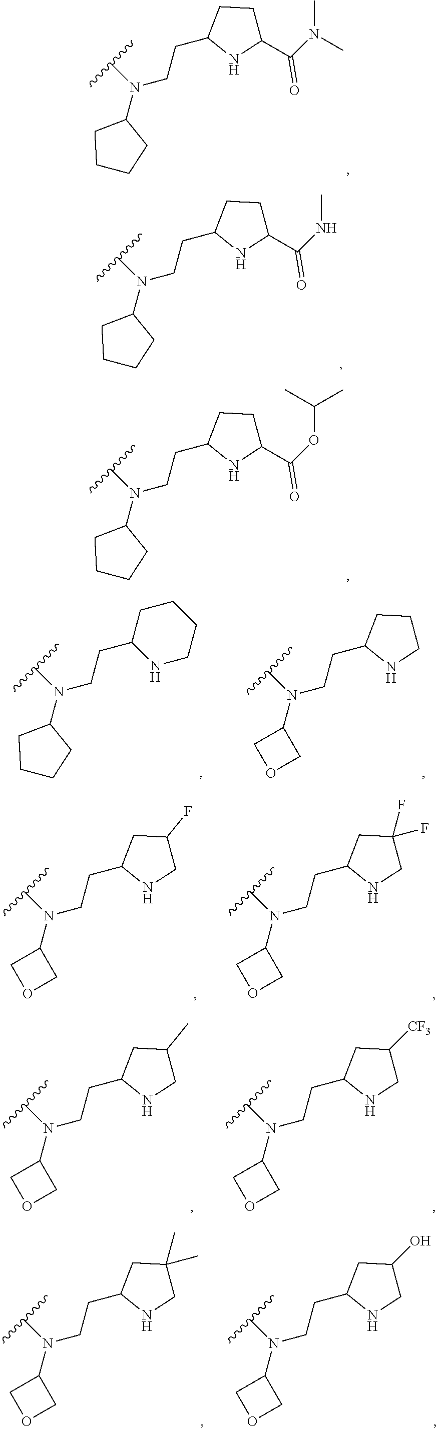

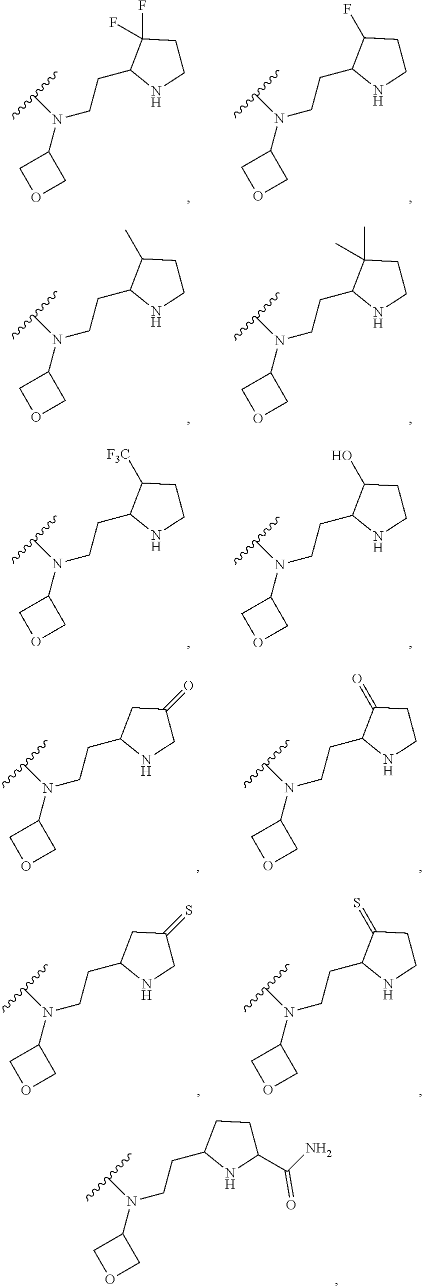

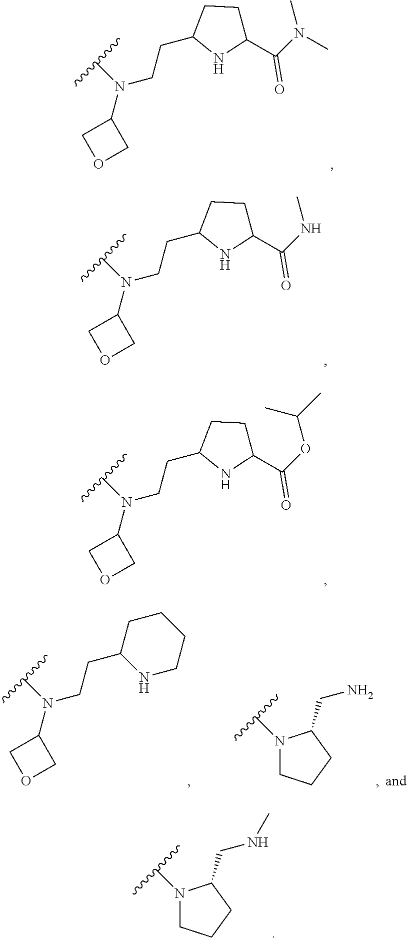

In some embodiments, the

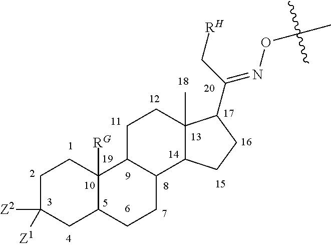

moiety is

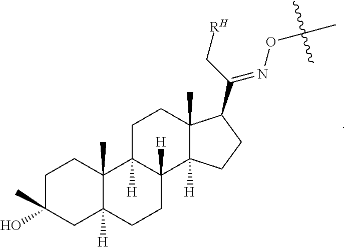

In some embodiments, the

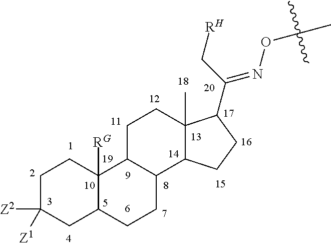

moiety is

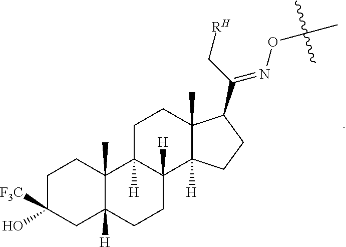

In some embodiments, the

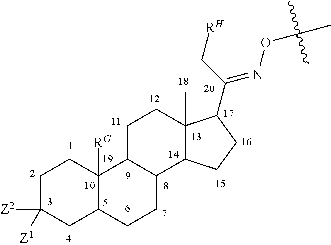

moiety is

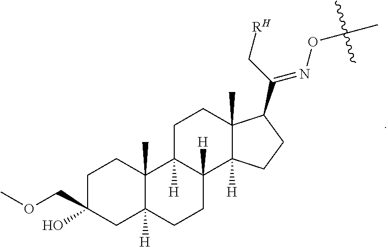

embodiments, the

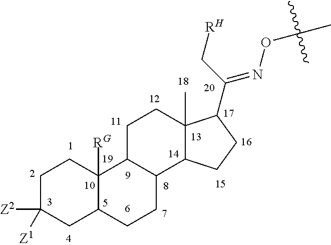

moiety is

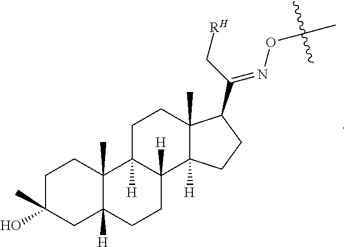

In some embodiments, the

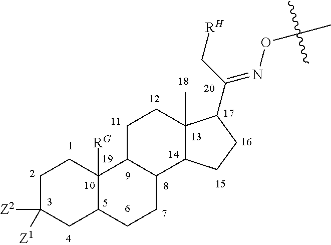

moiety is

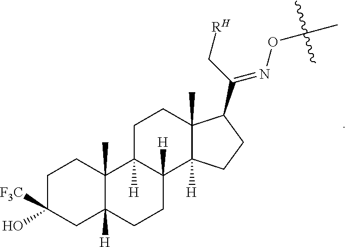

In some embodiments, the

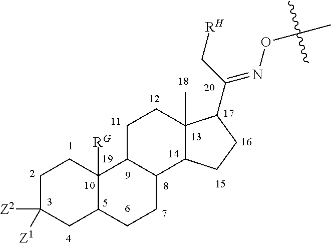

moiety is

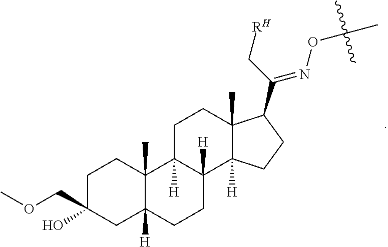

In some embodiments, the

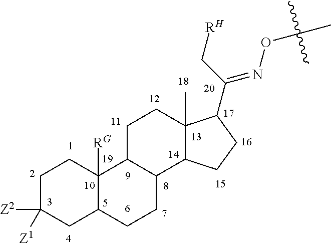

moiety is

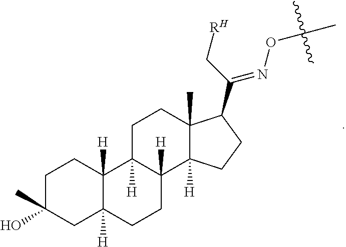

In some embodiments, the

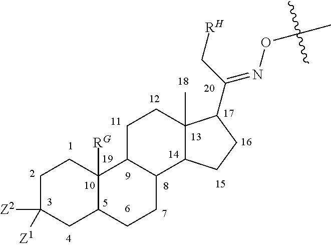

moiety is

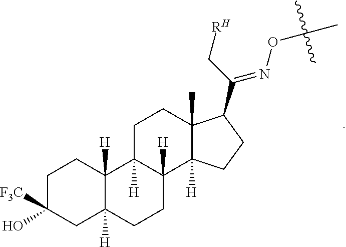

In some embodiments, the

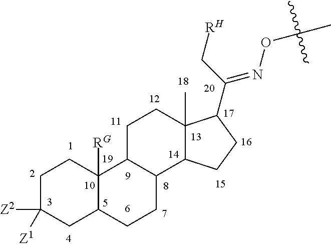

moiety is

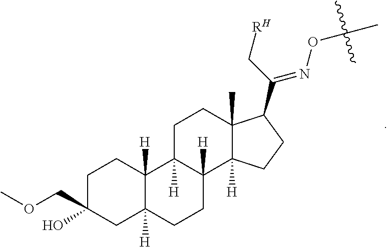

In some embodiments, the

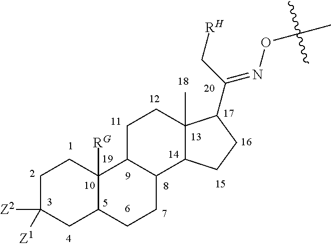

moiety is

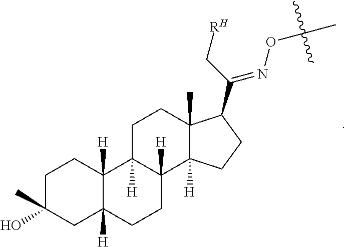

In some embodiments, the

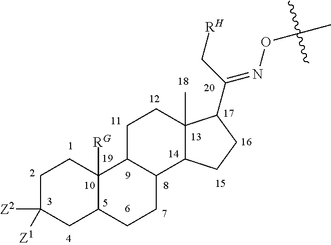

moiety is

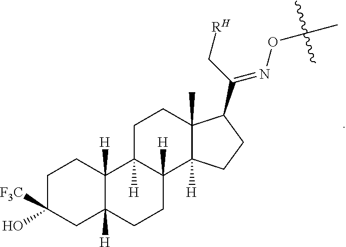

In some embodiments, the

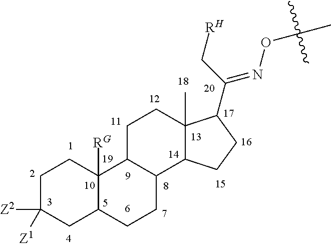

moiety is

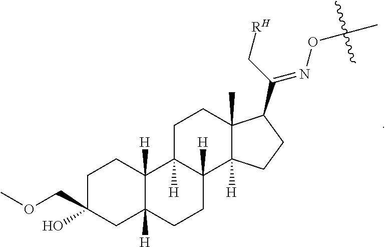

In some embodiments R^{H} is H. In some embodiments R^{H} is

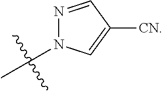 [SMILES: *n1cc(C#N)cn1]

In some embodiments, R^{H} is

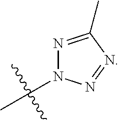 [SMILES: *n1nnc(C)n1]

In some embodiments, R^{H} is

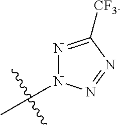 [SMILES: *n1nnc(C(F)(F)F)n1]

In some embodiments, R^{H} is

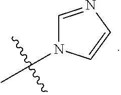 [SMILES: *n1ccnc1]

In some embodiments, the

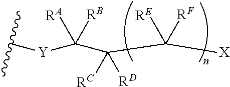

moiety is selected from

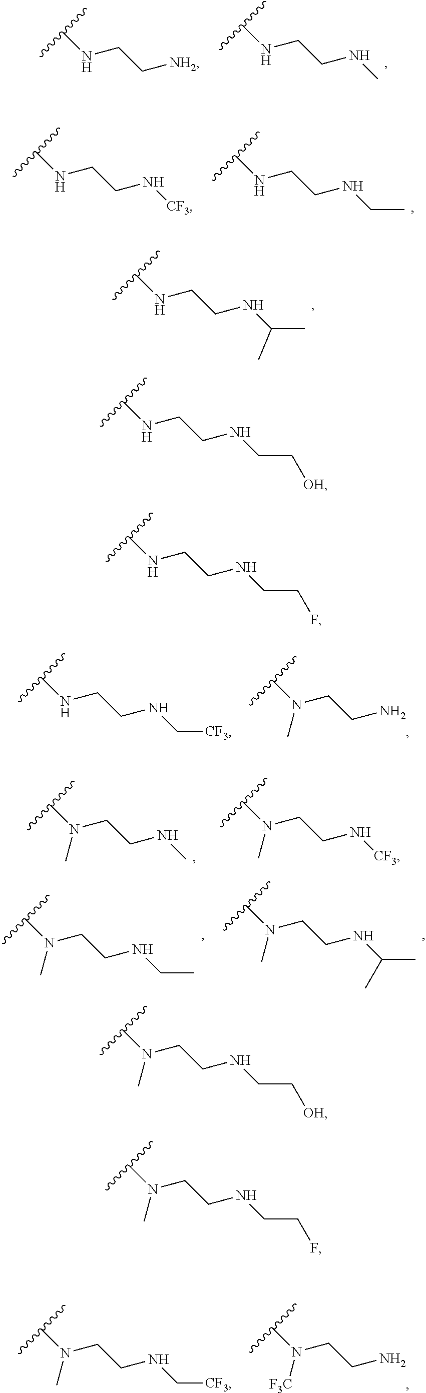

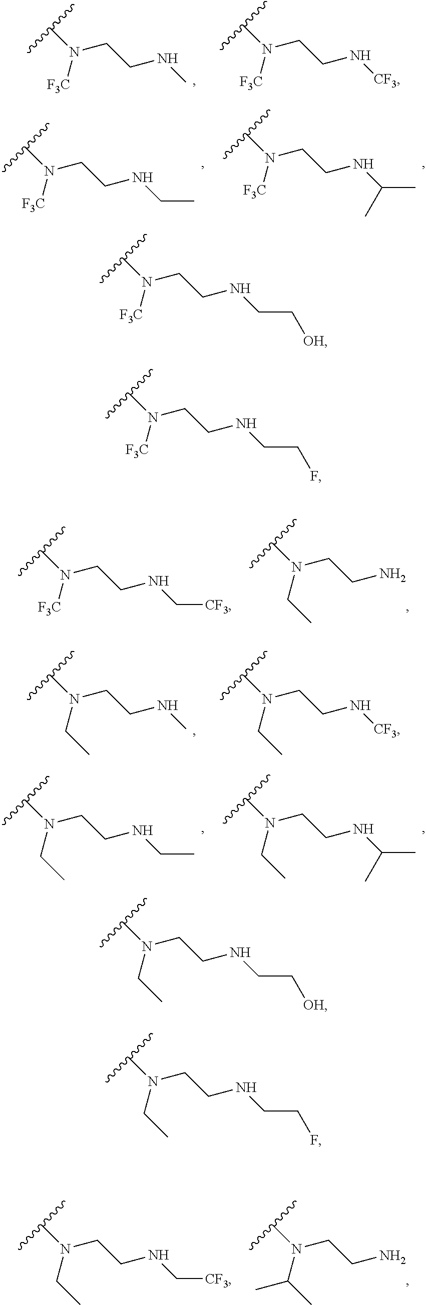

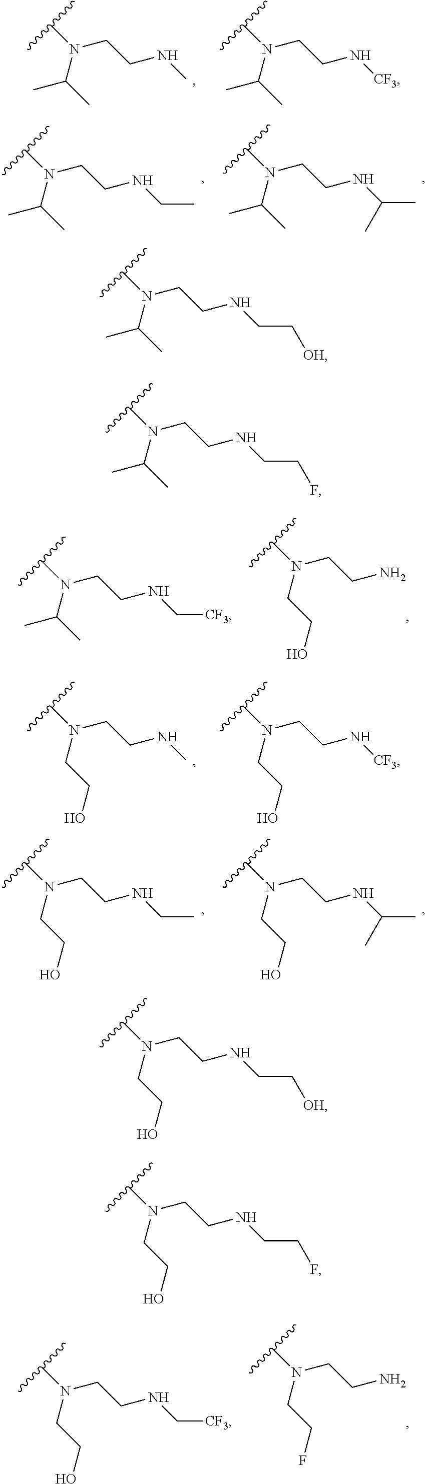

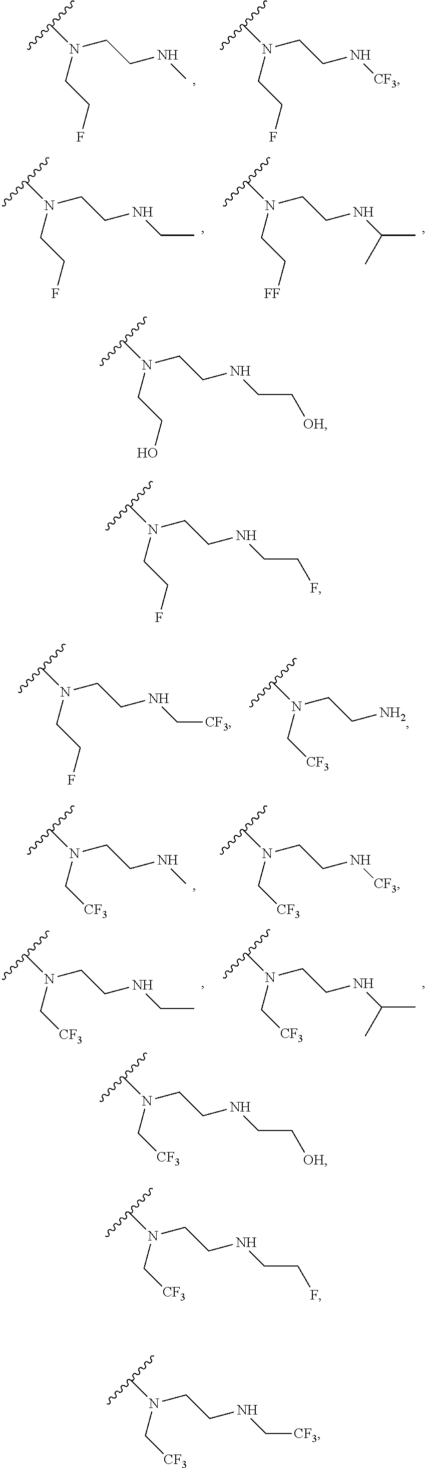

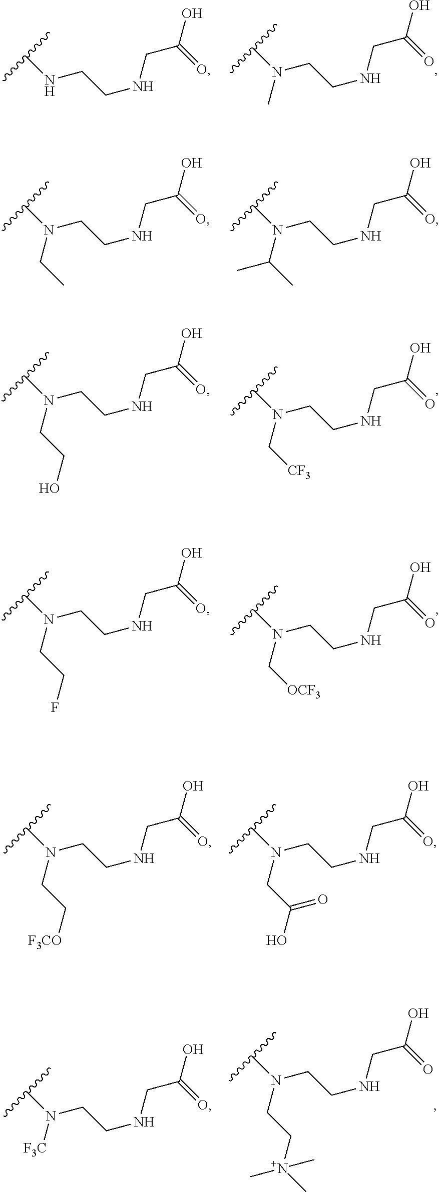

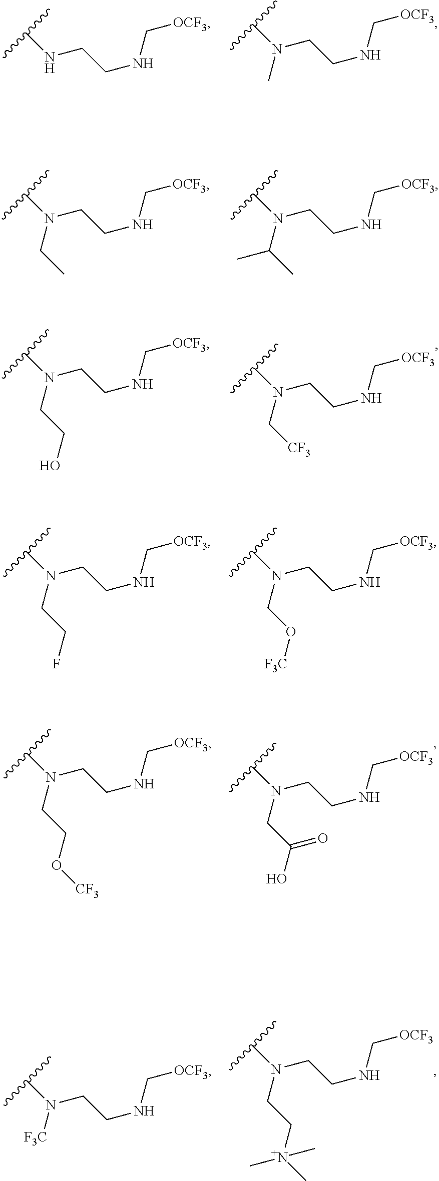

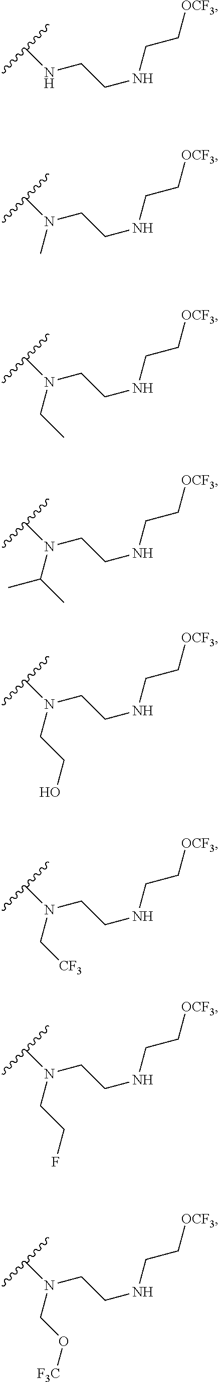

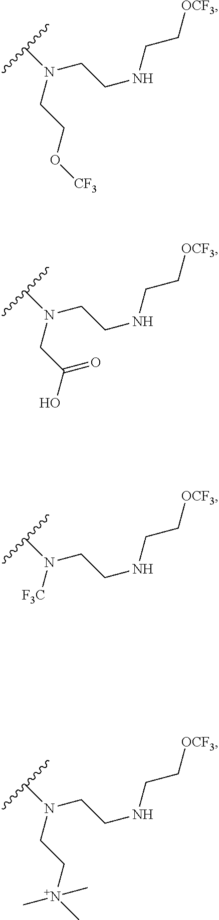 [SMILES: *N(CCNCCOC(F)(F)F)CCOC(F)(F)F ; *N(CCNCCOC(F)(F)F)CC(=O)O ; *N(CCNCCOC(F)(F)F)C(F)(F)F ; *N(CCNCCOC(F)(F)F)CC[N+](C)(C)C]

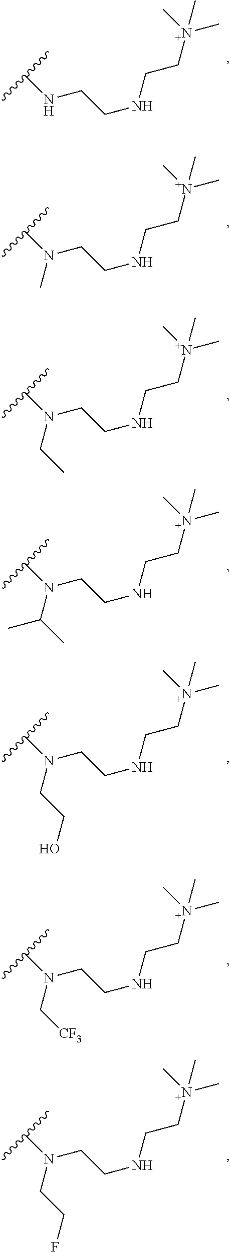

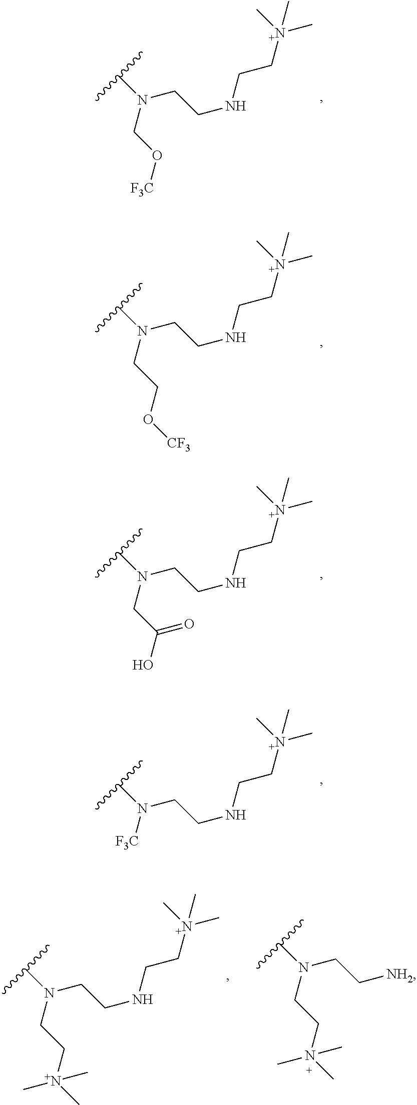

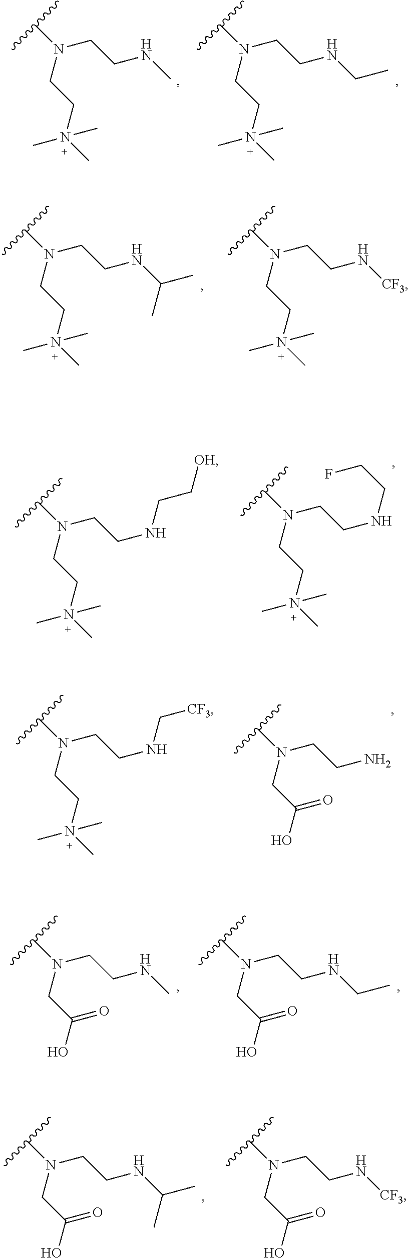

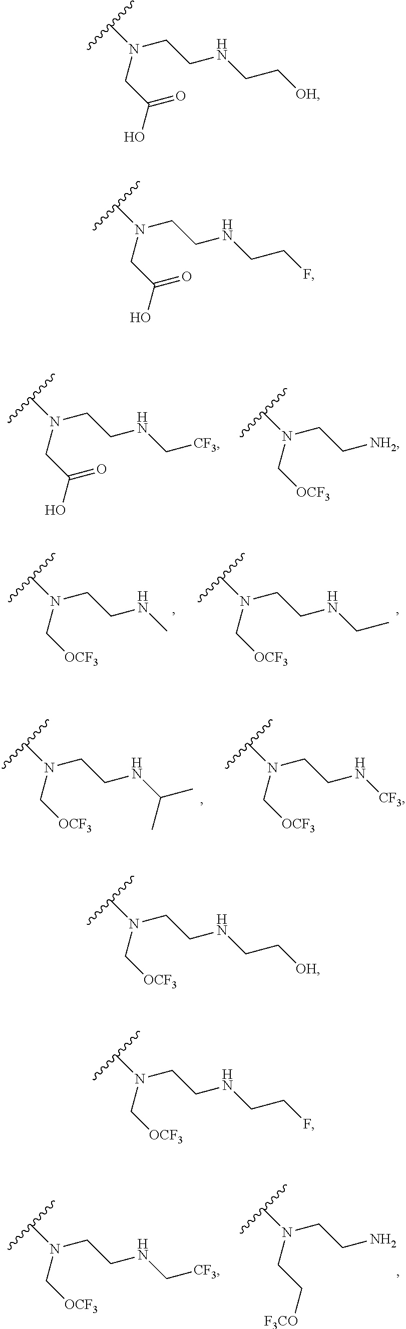

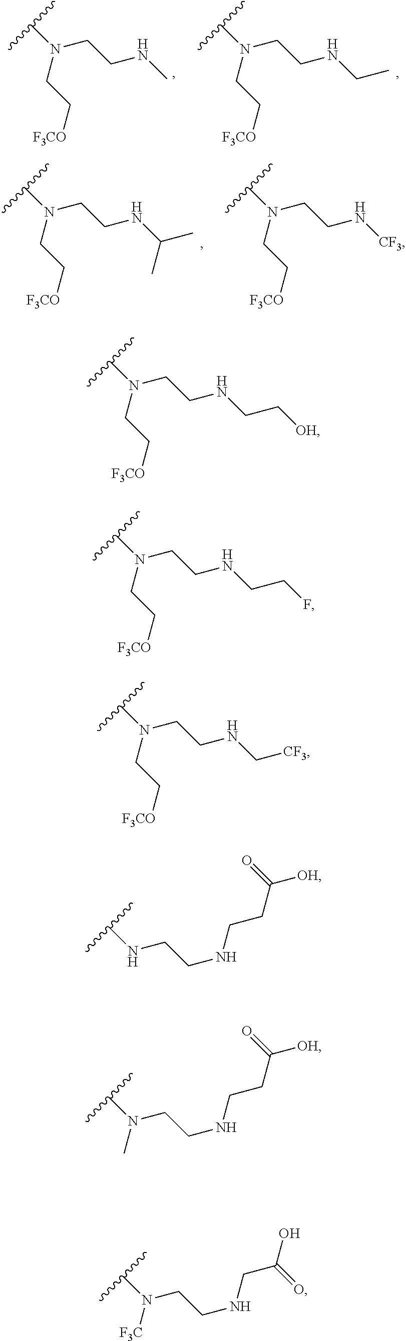

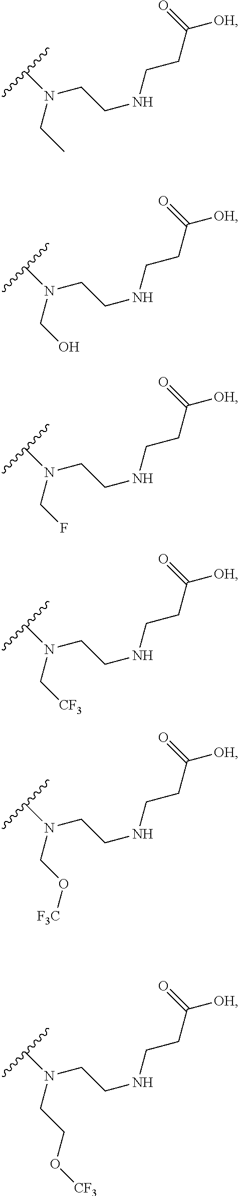 [SMILES: *N(CC)CCNCCC(=O)O ; *N(CO)CCNCCC(=O)O ; *N(CF)CCNCCC(=O)O ; *N(CCNCCC(=O)O)CC(F)(F)F ; *N(CCNCCC(=O)O)COC(F)(F)F ; *N(CCNCCC(=O)O)CCOC(F)(F)F]

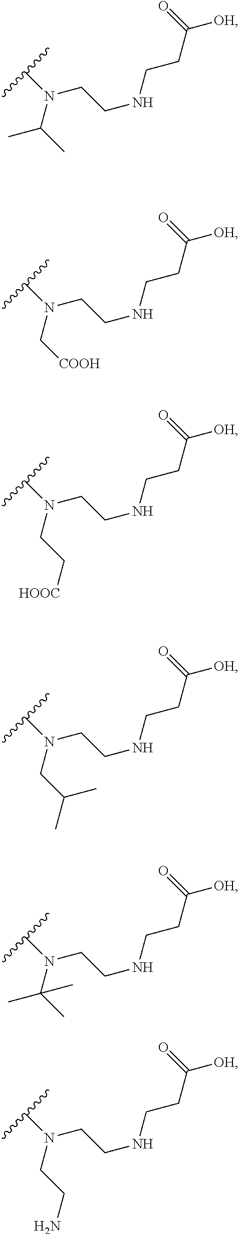 [SMILES: *N(CCNCCC(=O)O)C(C)C ; *N(CCNCCC(=O)O)CC(=O)O ; *N(CCNCCC(=O)O)CCC(=O)O ; *N(CCNCCC(=O)O)CC(C)C ; *N(CCNCCC(=O)O)C(C)(C)C ; *N(CCN)CCNCCC(=O)O]

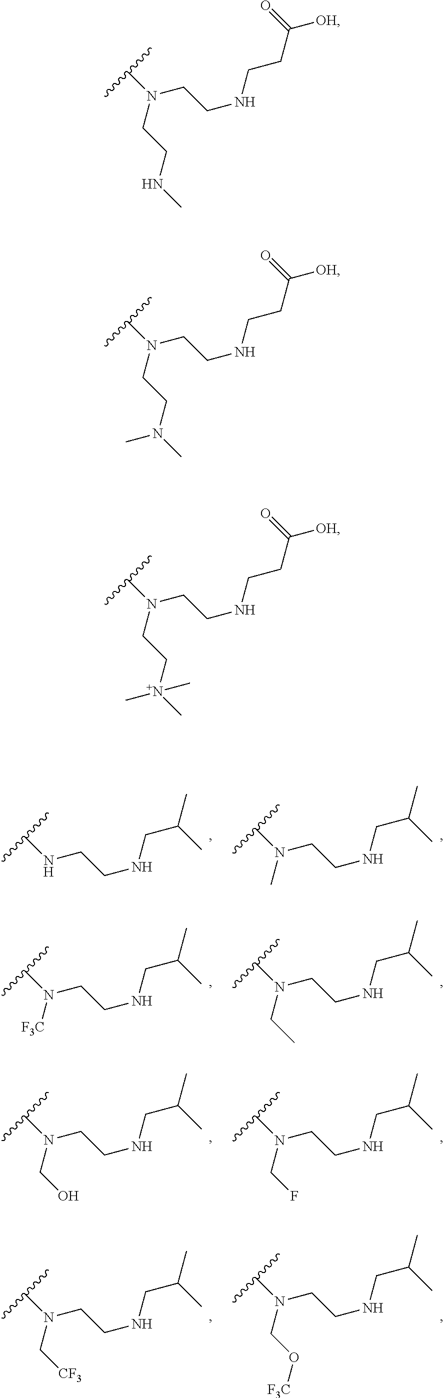

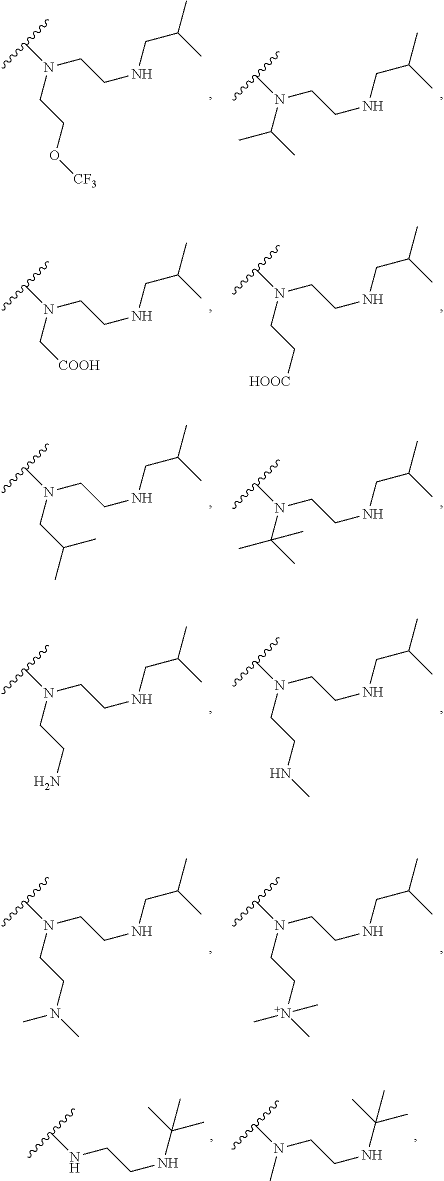

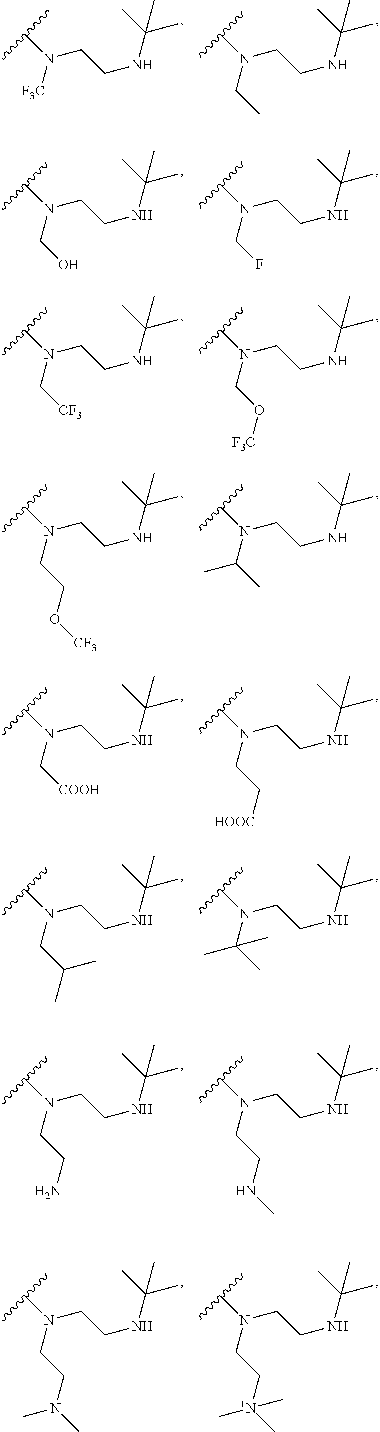

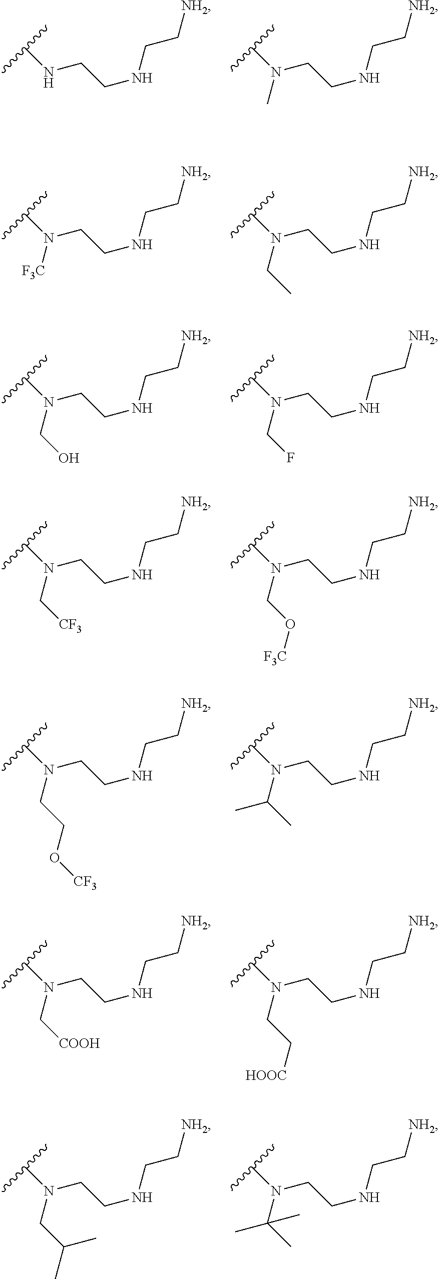

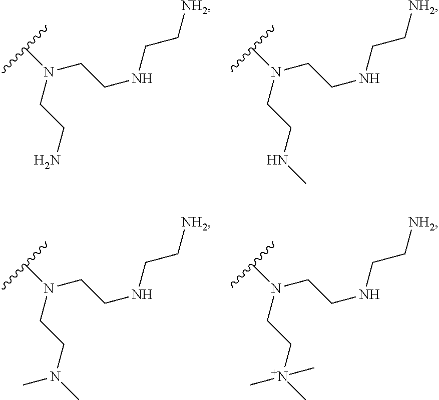 [SMILES: *N(CCN)CCNCCN ; *N(CCNC)CCNCCN ; *N(CCNCCN)CCN(C)C ; *N(CCNCCN)CC[N+](C)(C)C]

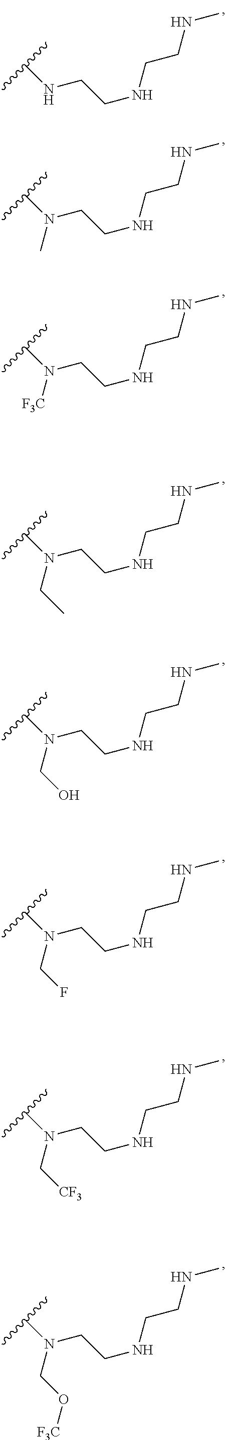

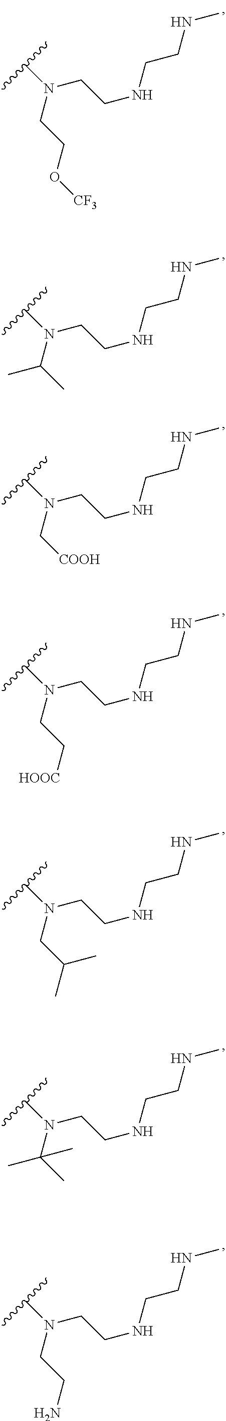

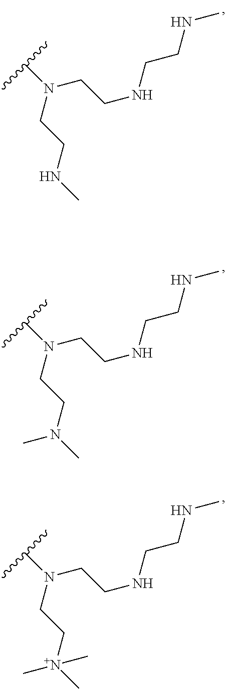

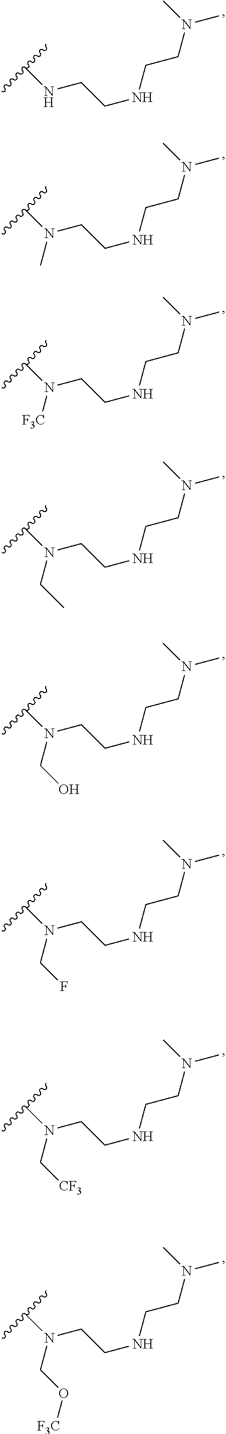

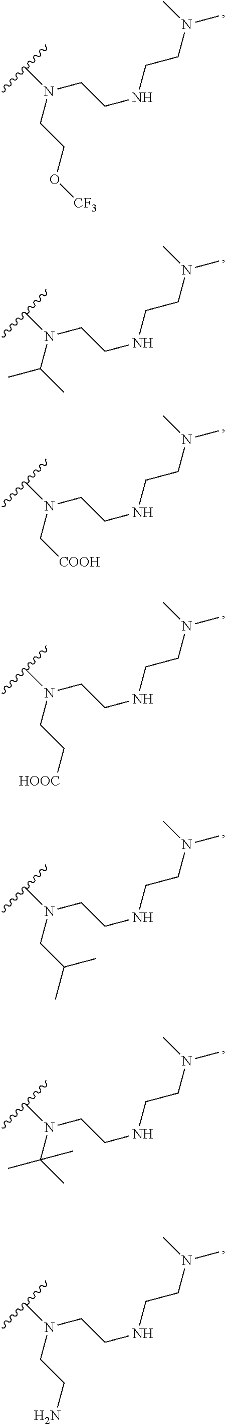

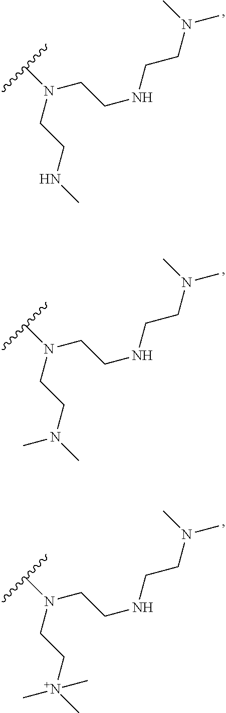

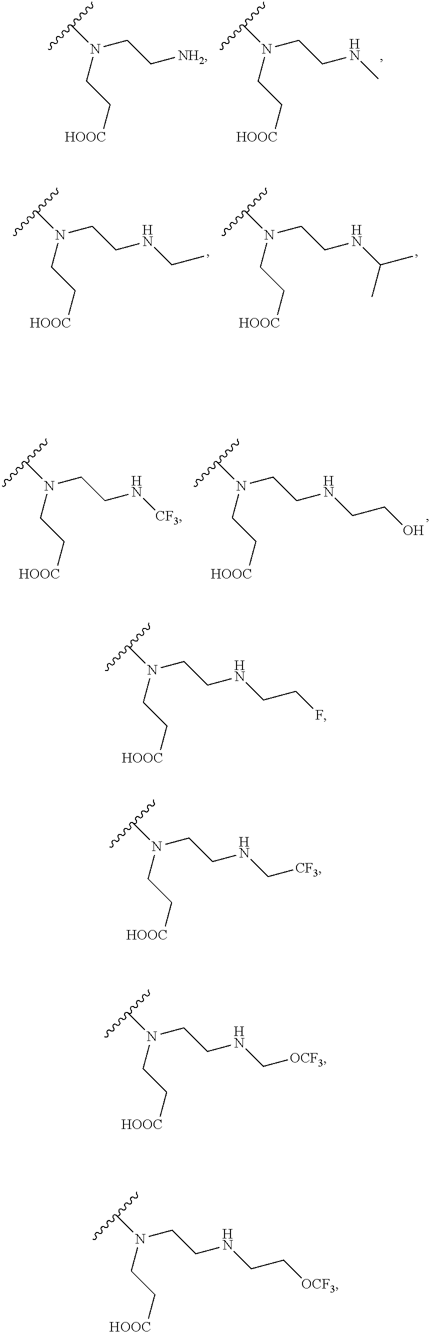

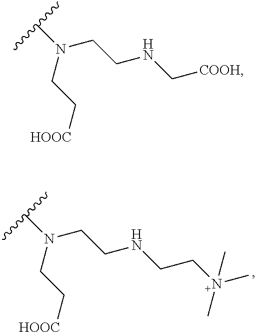 [SMILES: *N(CCNCC(=O)O)CCC(=O)O ; *N(CCNCC[N+](C)(C)C)CCC(=O)O]

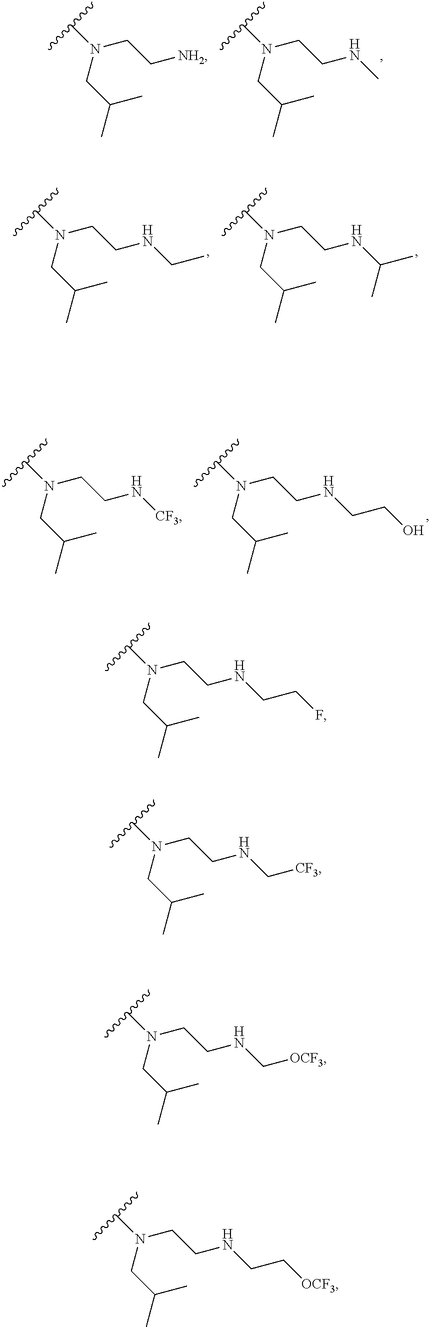

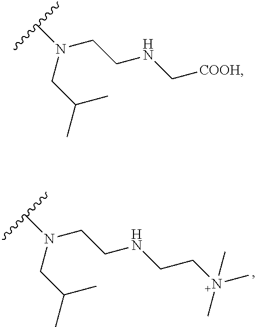 [SMILES: *N(CCNCC(=O)O)CC(C)C ; *N(CCNCC[N+](C)(C)C)CC(C)C]

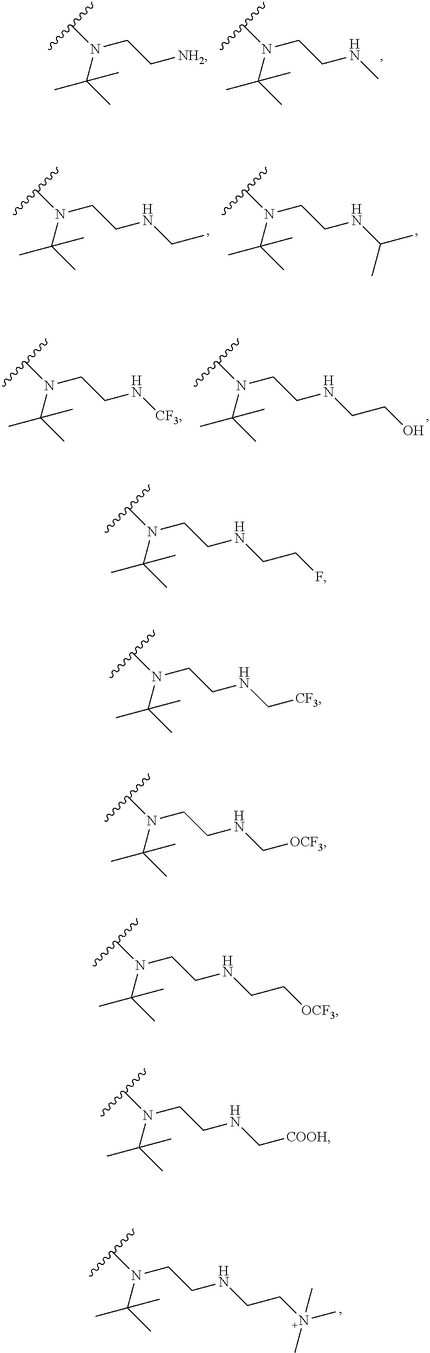

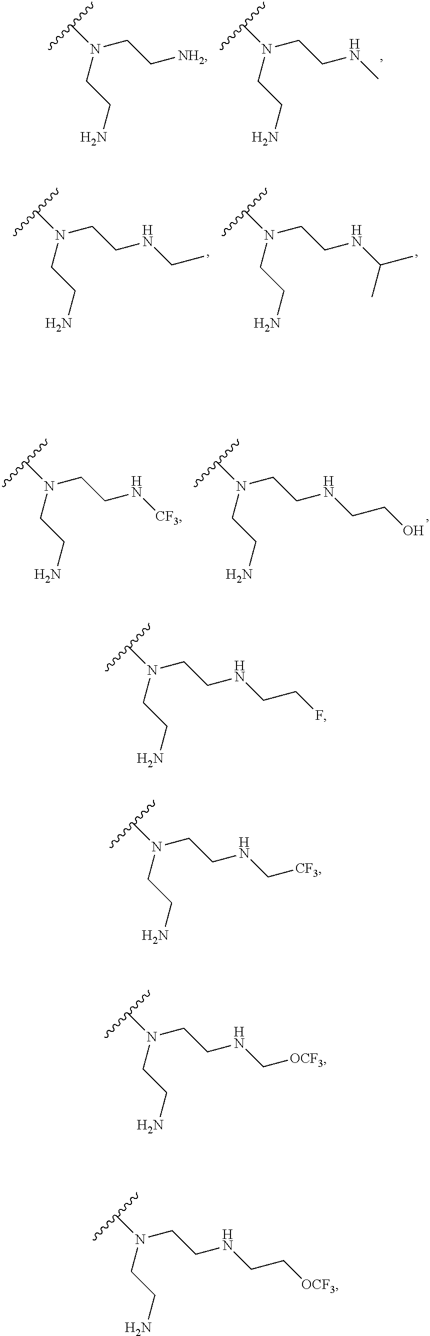

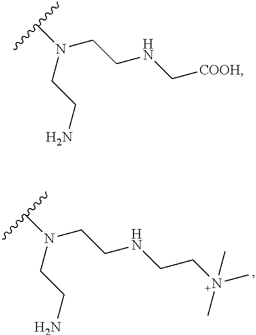 [SMILES: *N(CCN)CCNCC(=O)O ; *N(CCN)CCNCC[N+](C)(C)C]

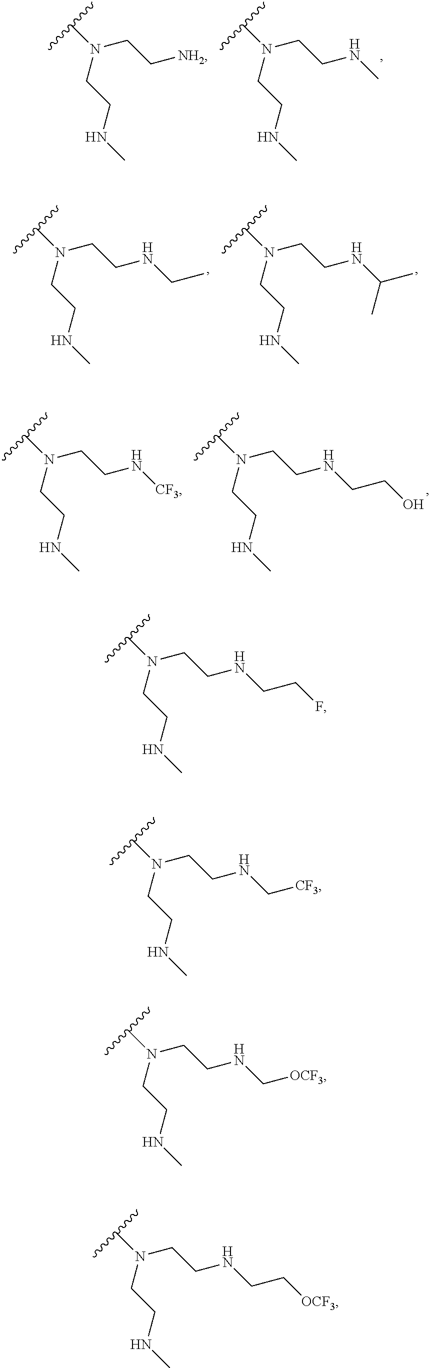

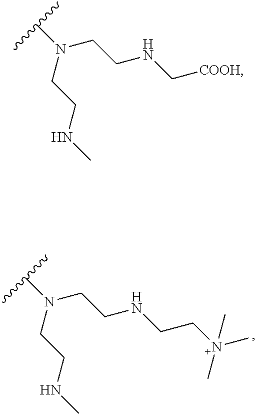 [SMILES: *N(CCNC)CCNCC(=O)O ; *N(CCNC)CCNCC[N+](C)(C)C]

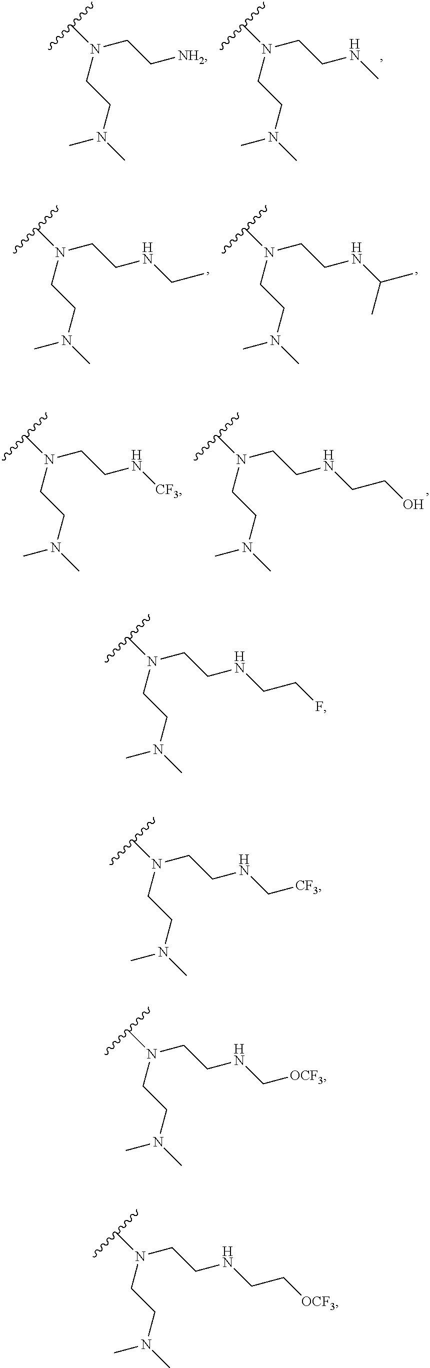

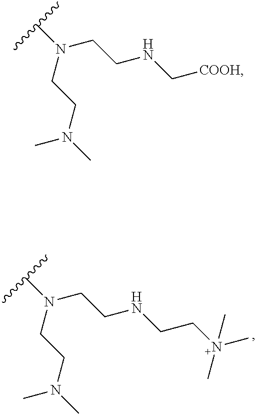

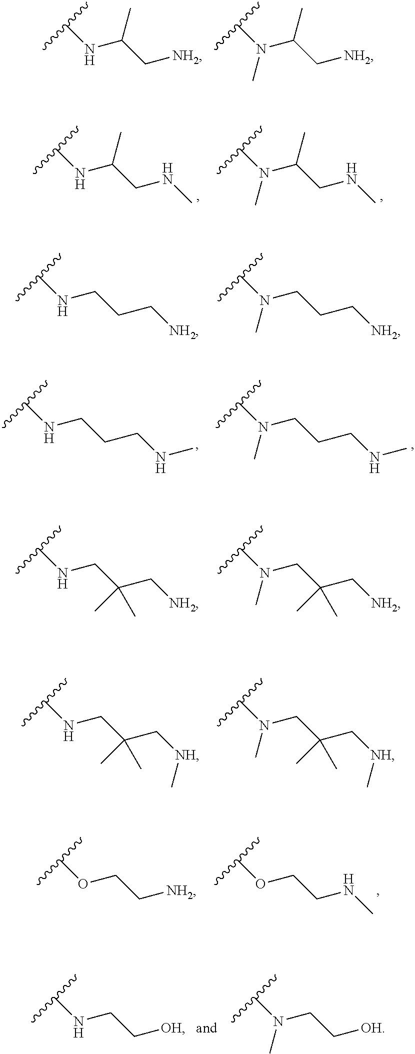

In some embodiments, the

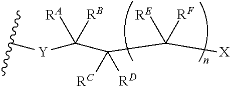

moiety is selected from

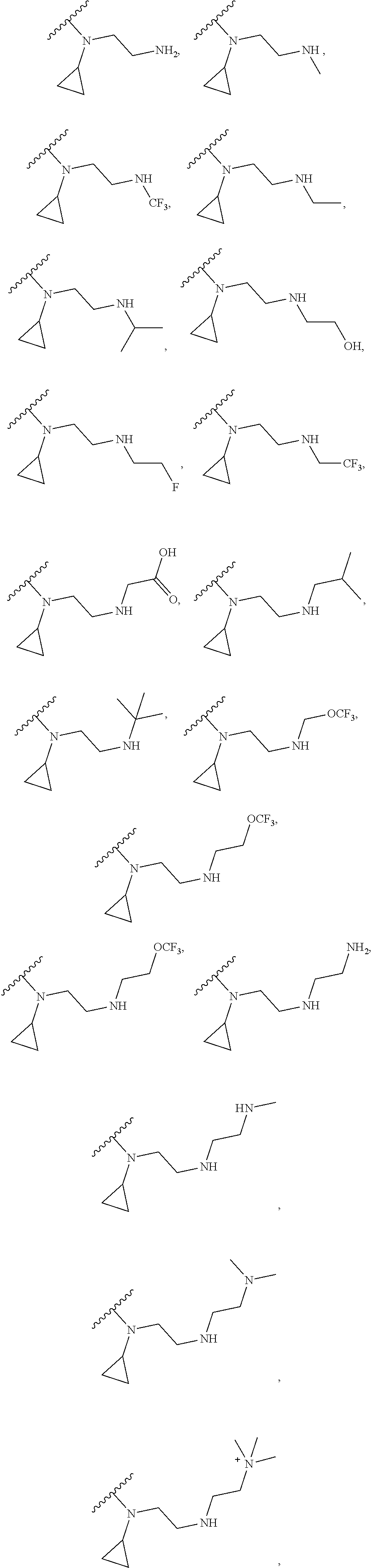

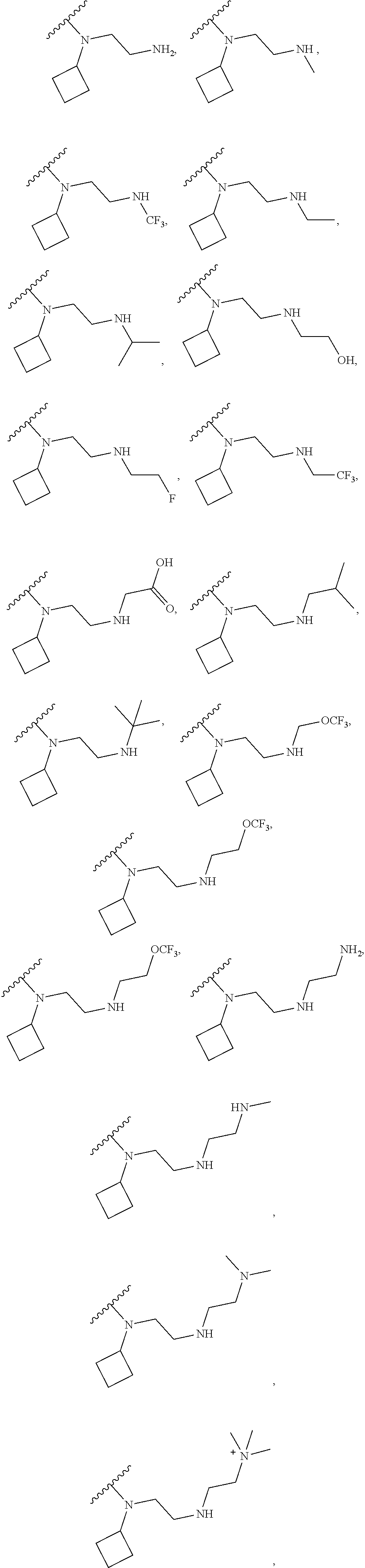

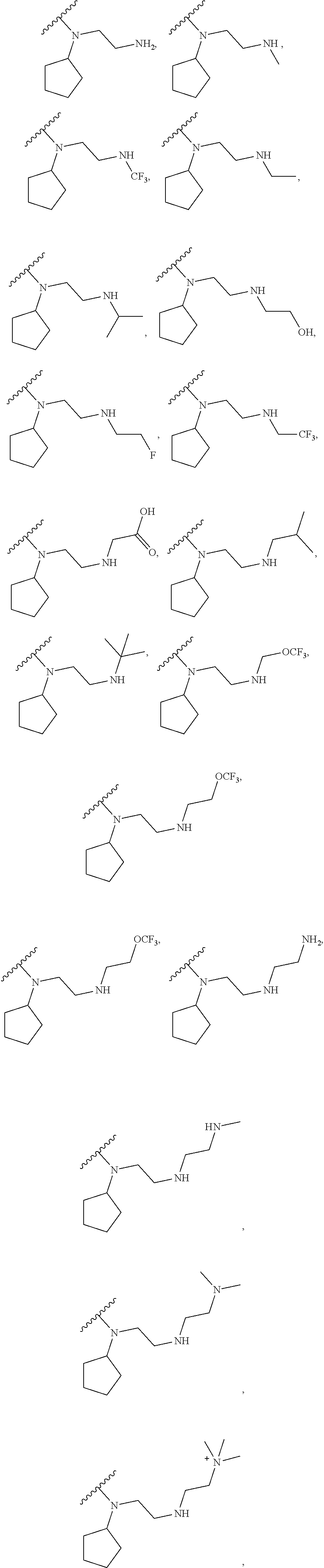

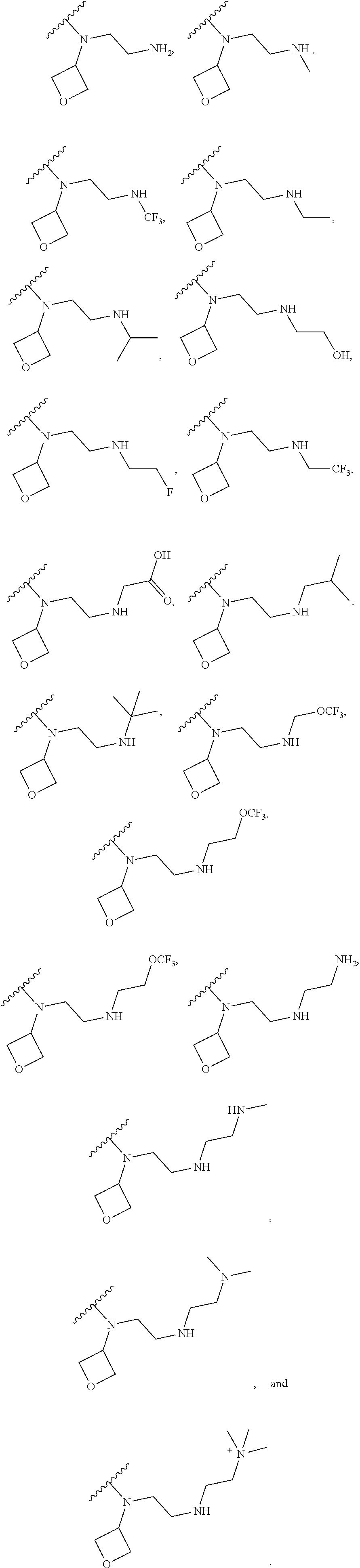

In some embodiments, the

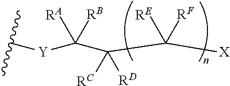

moiety is selected from

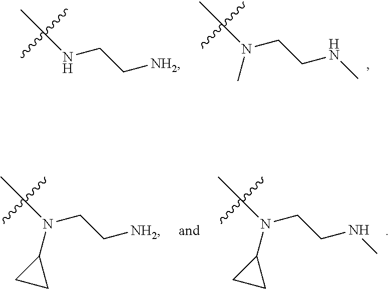

In some embodiments, the

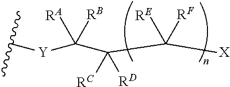

moiety is

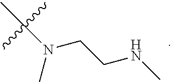 [SMILES: *N(C)CCNC]

In some embodiments, the

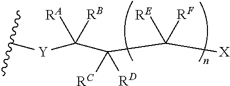

moiety is selected from

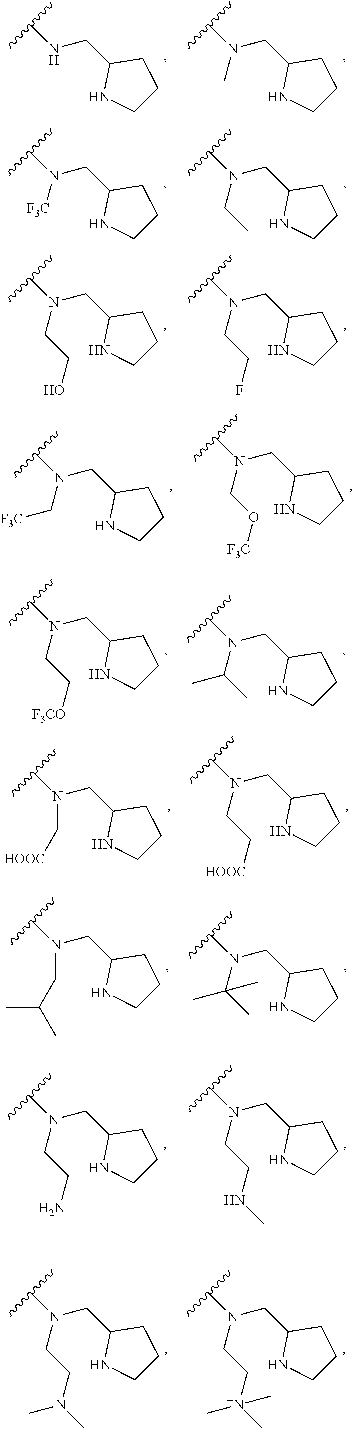

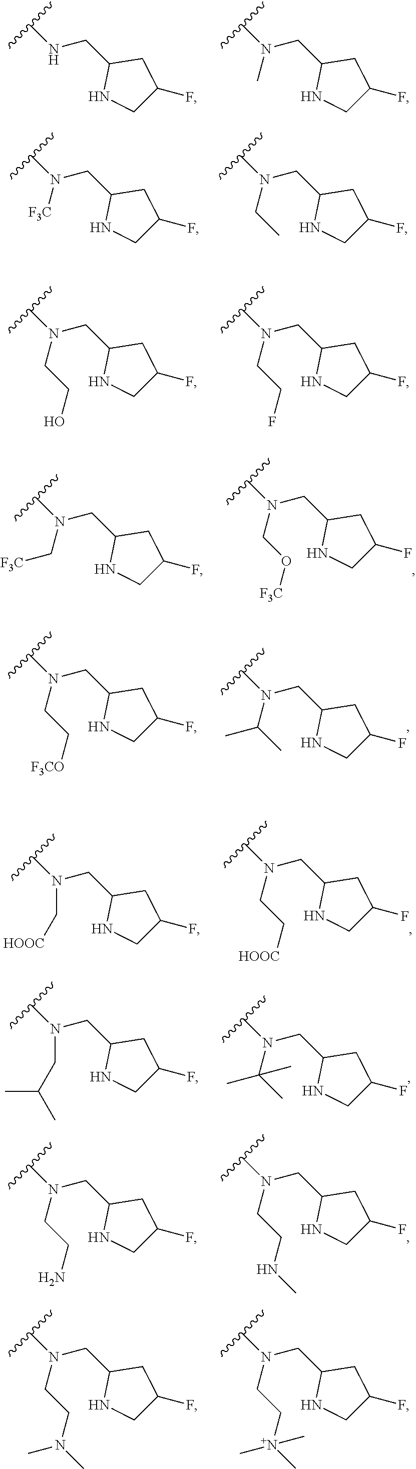

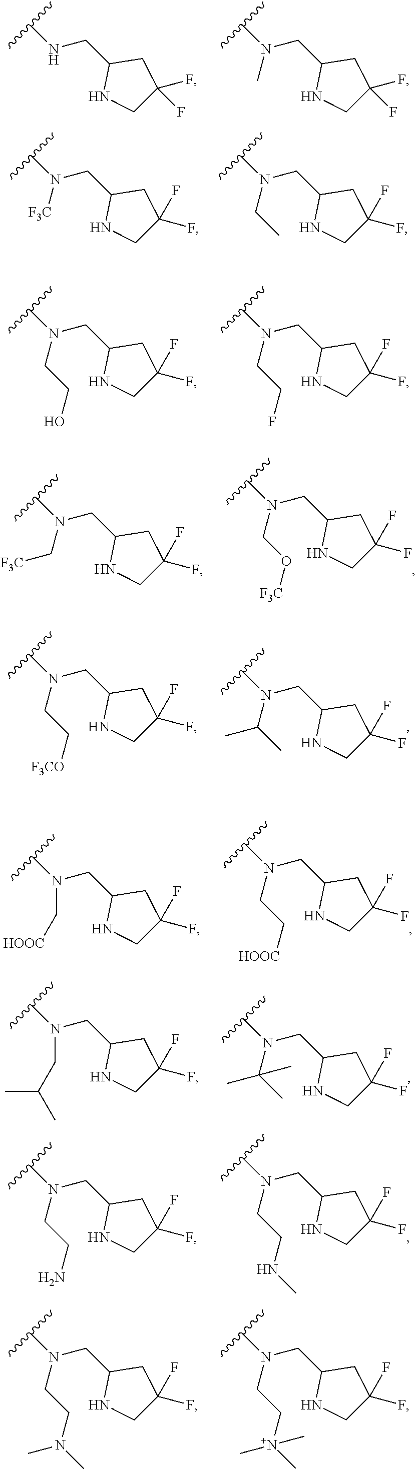

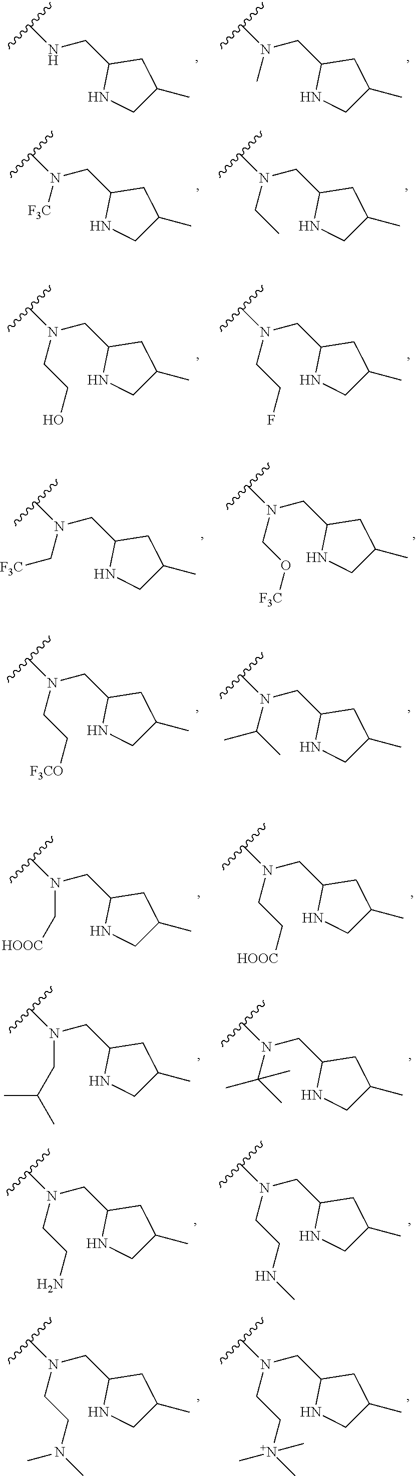

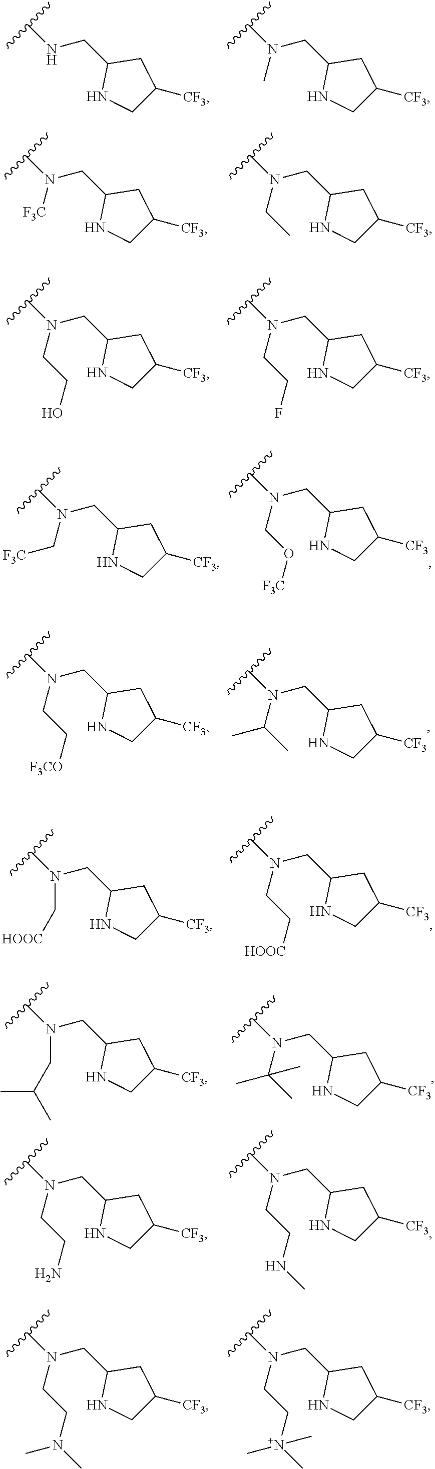

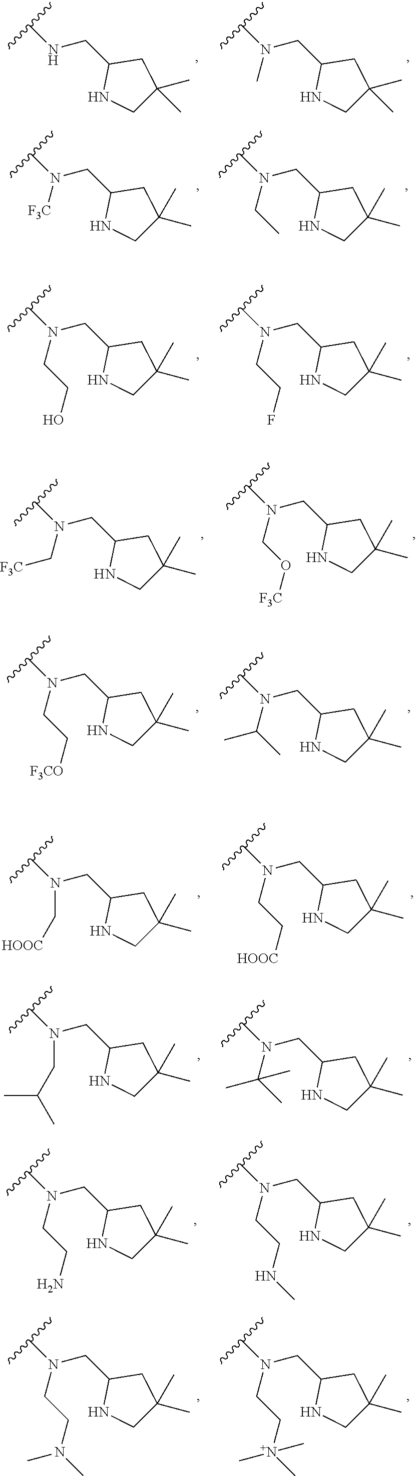

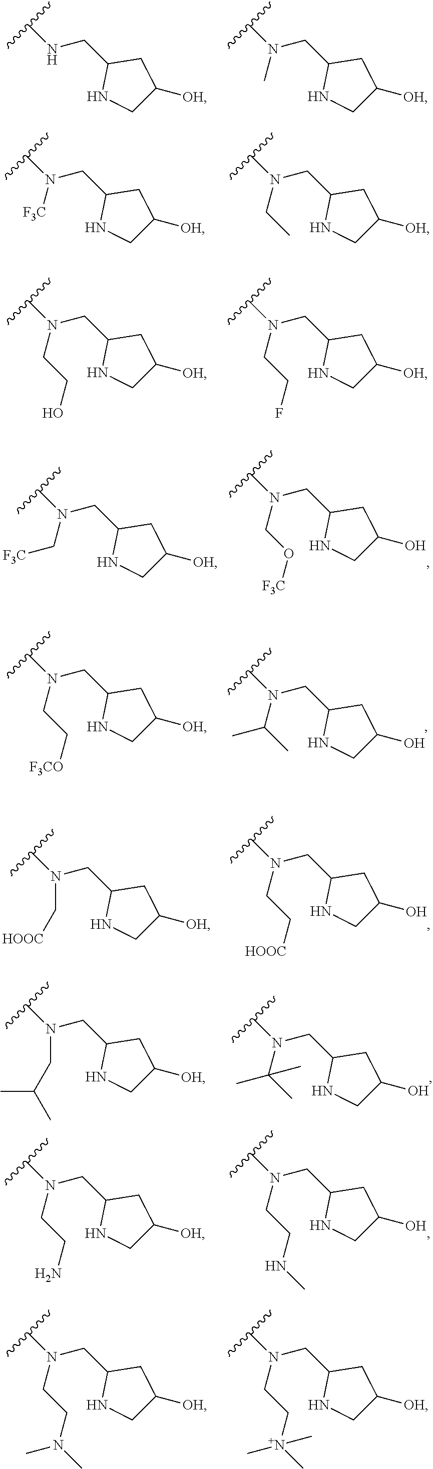

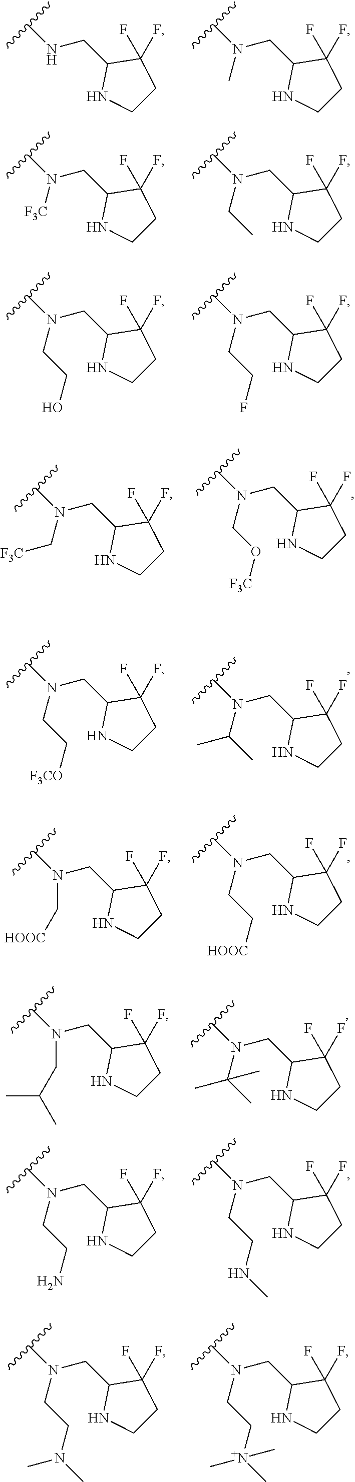

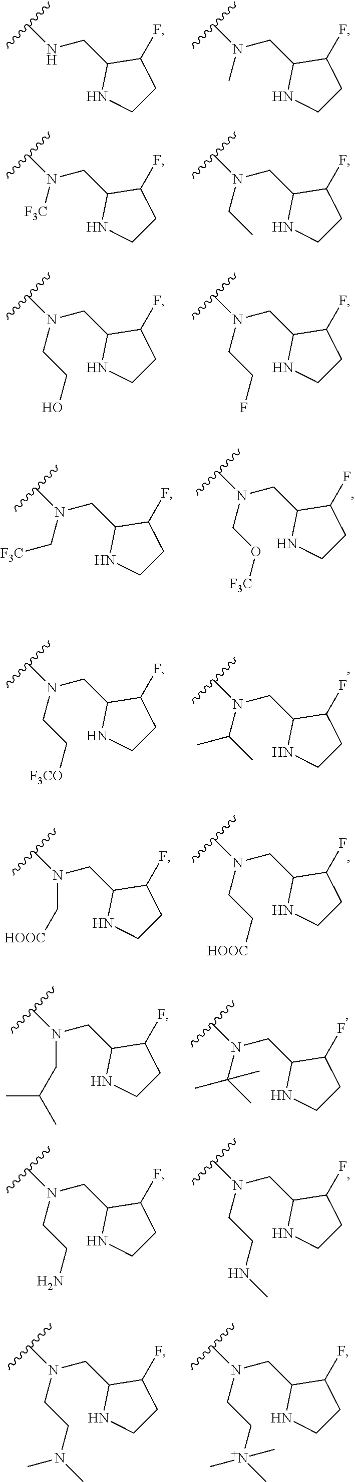

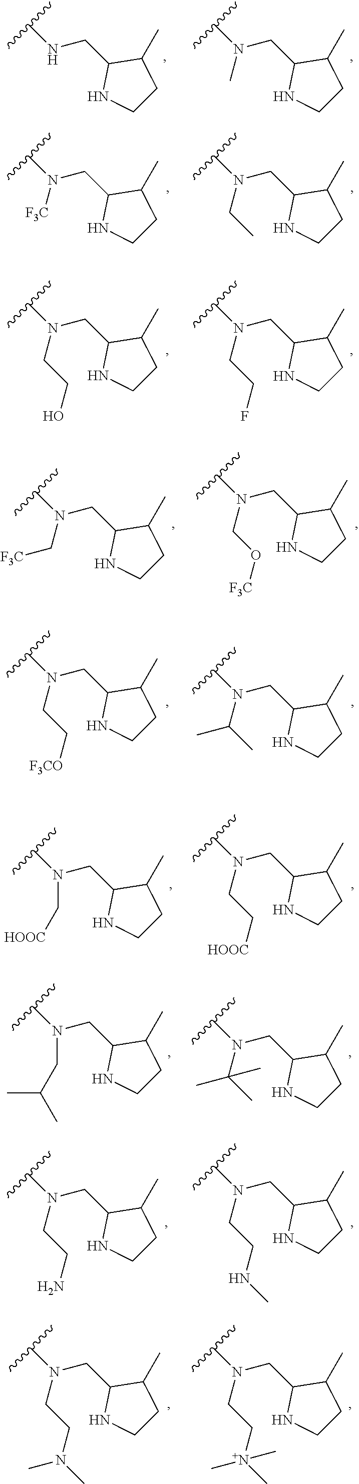

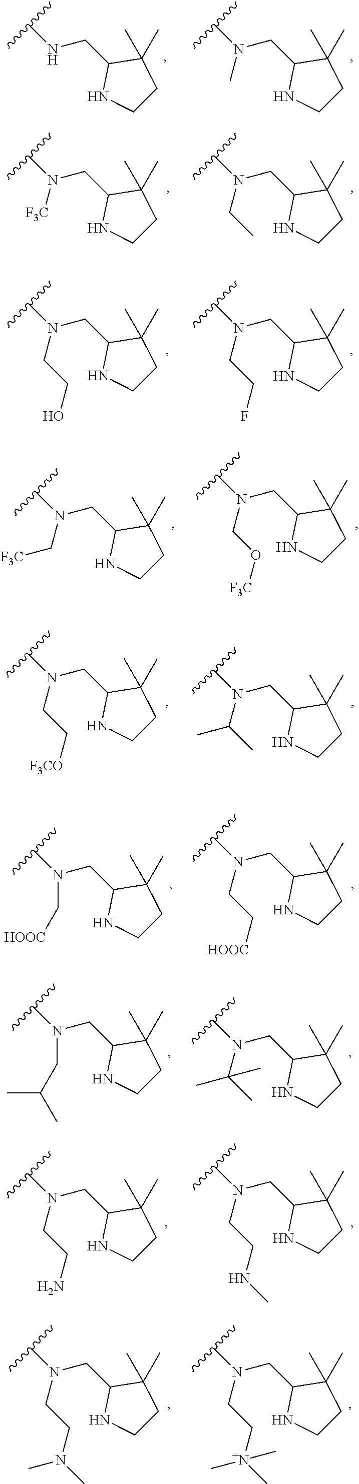

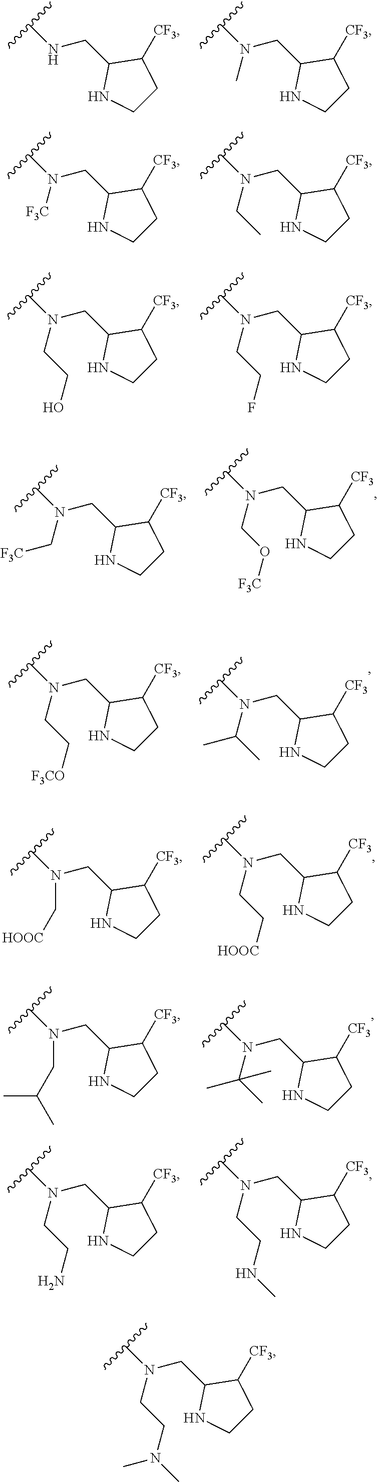

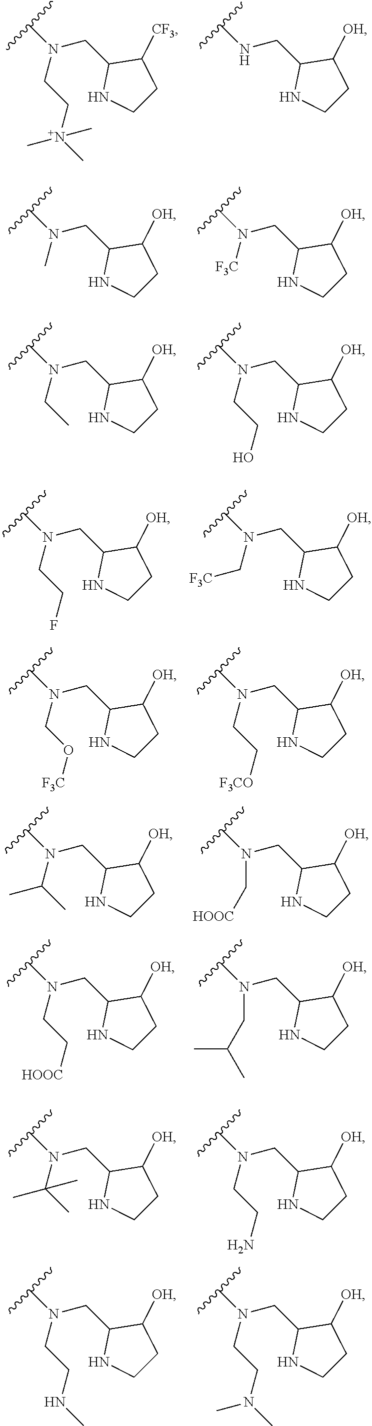

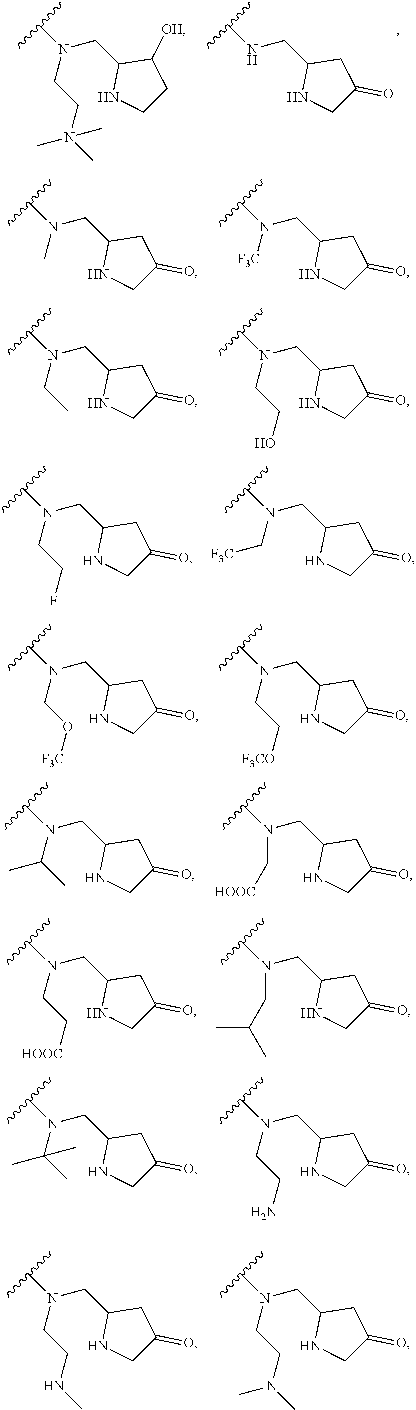

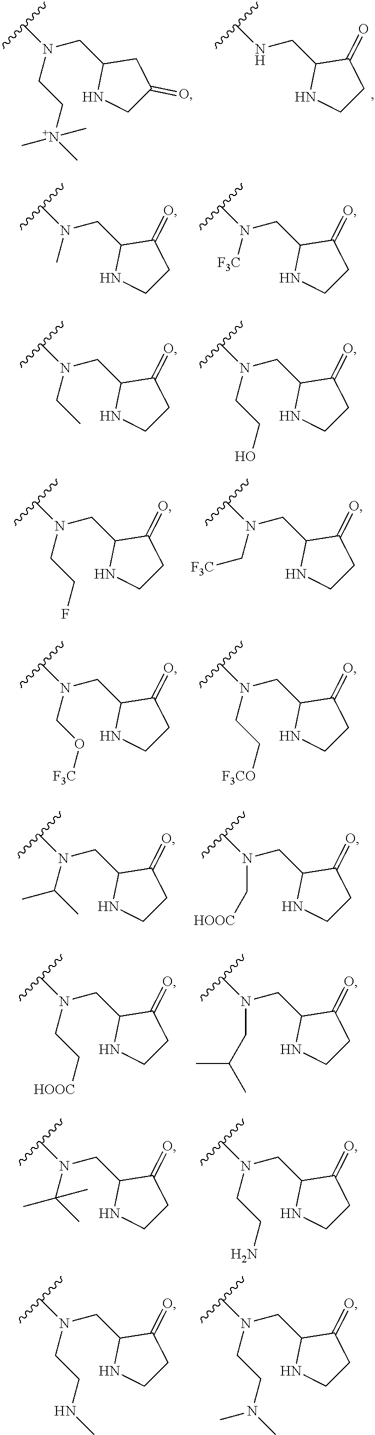

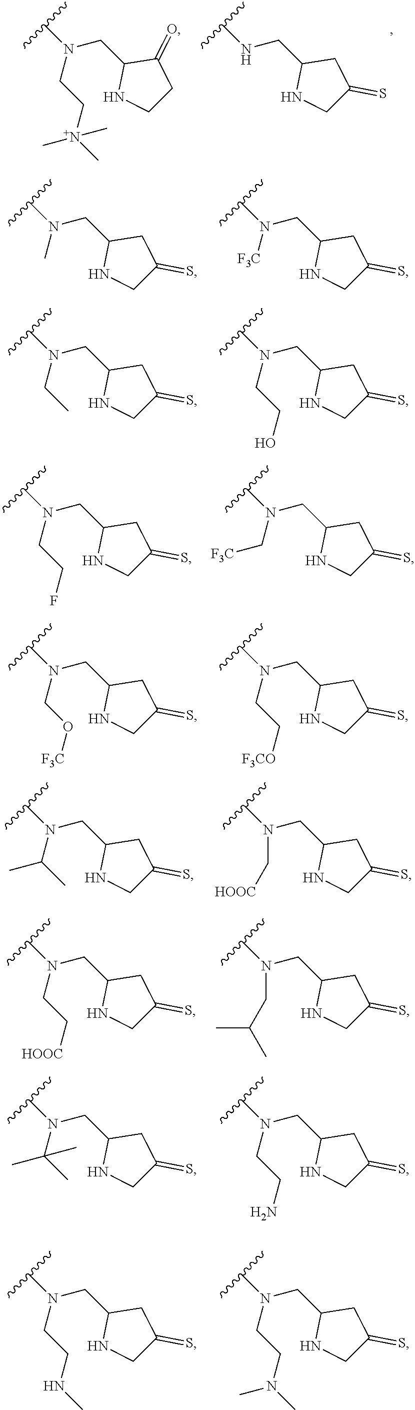

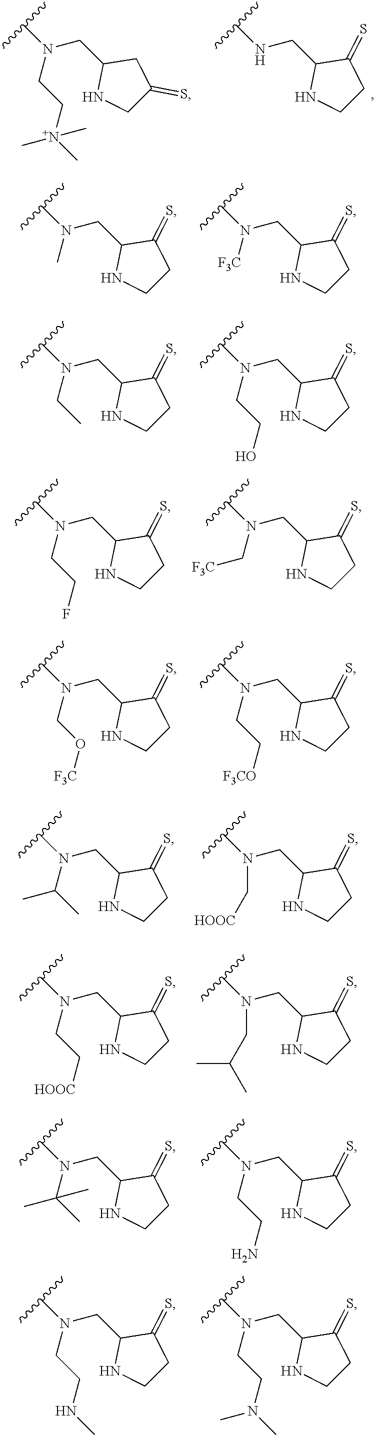

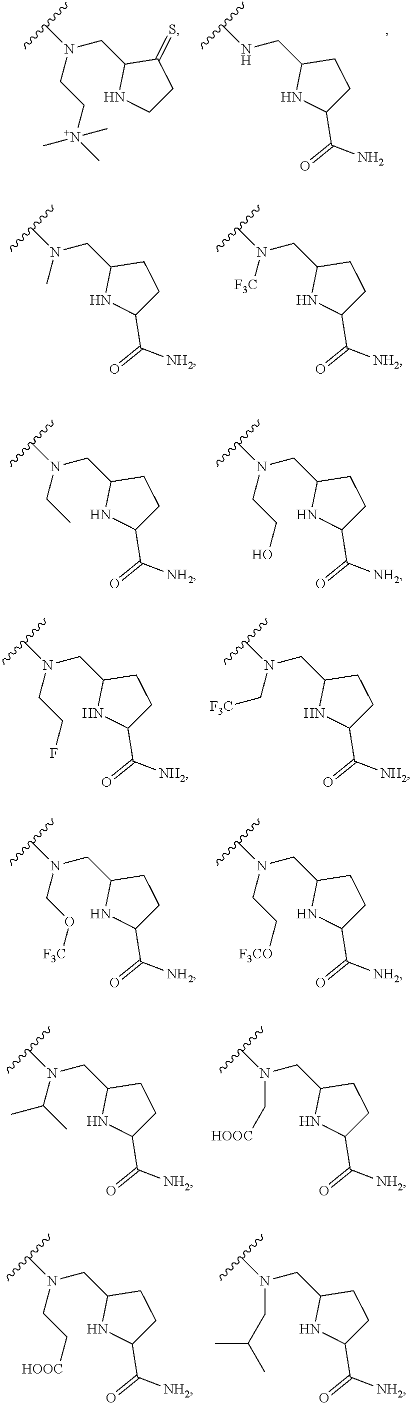

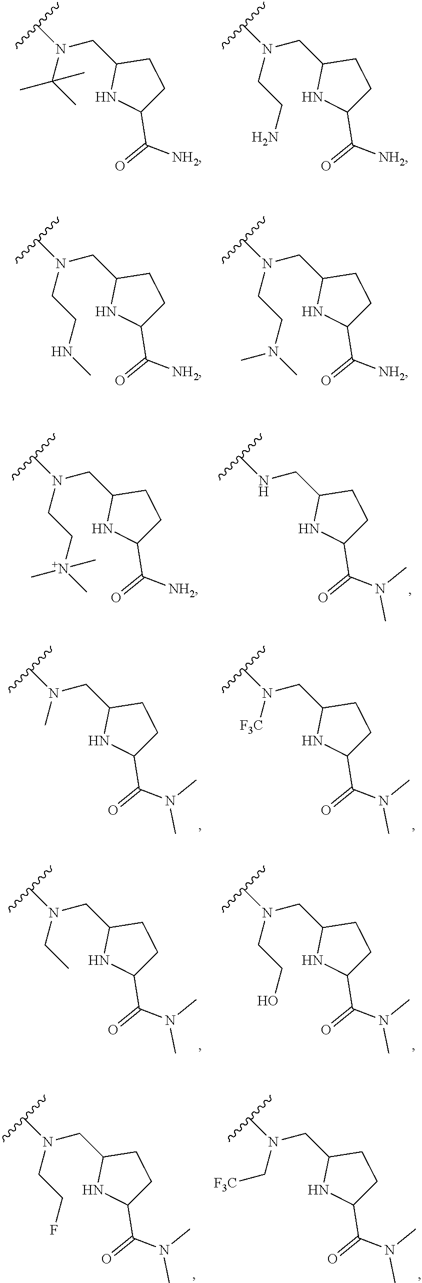

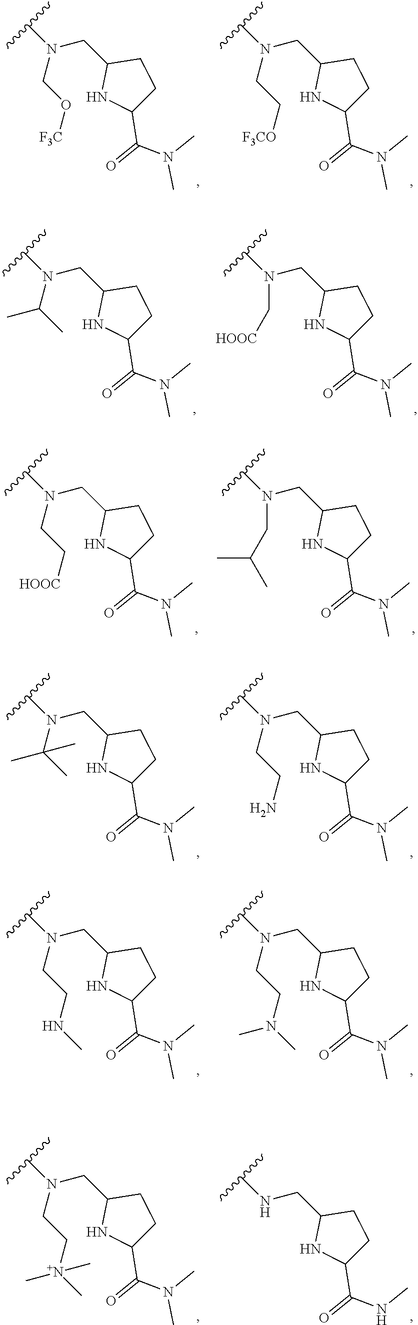

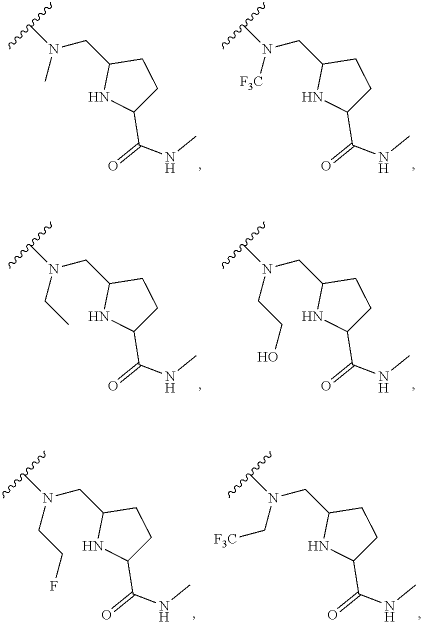 [SMILES: *N(C)CC1CCC(C(=O)NC)N1 ; *N(CC1CCC(C(=O)NC)N1)C(F)(F)F ; *N(CC)CC1CCC(C(=O)NC)N1 ; *N(CCO)CC1CCC(C(=O)NC)N1 ; *N(CCF)CC1CCC(C(=O)NC)N1 ; *N(CC1CCC(C(=O)NC)N1)CC(F)(F)F]

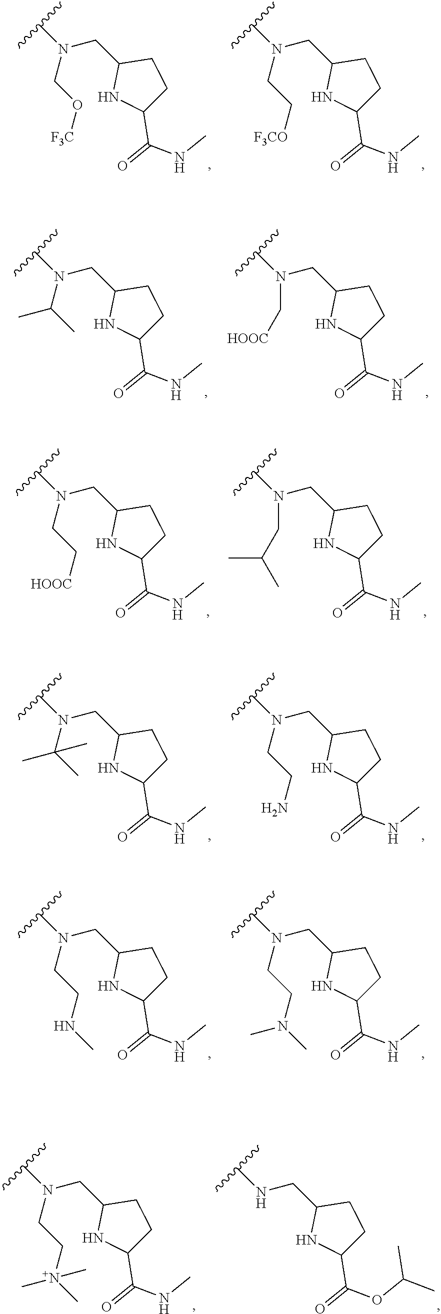

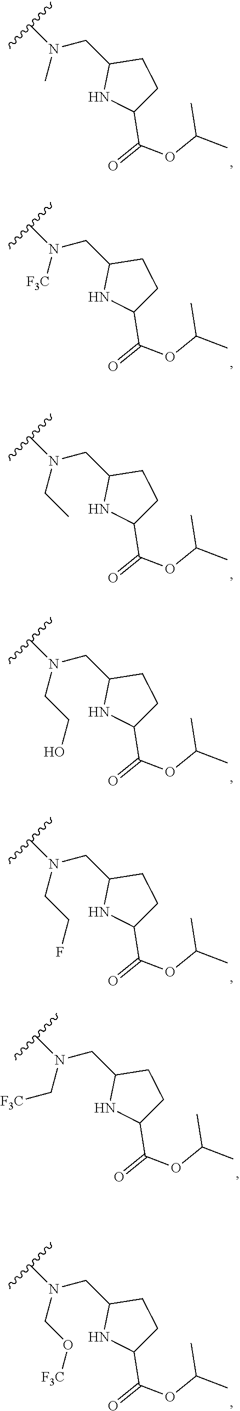

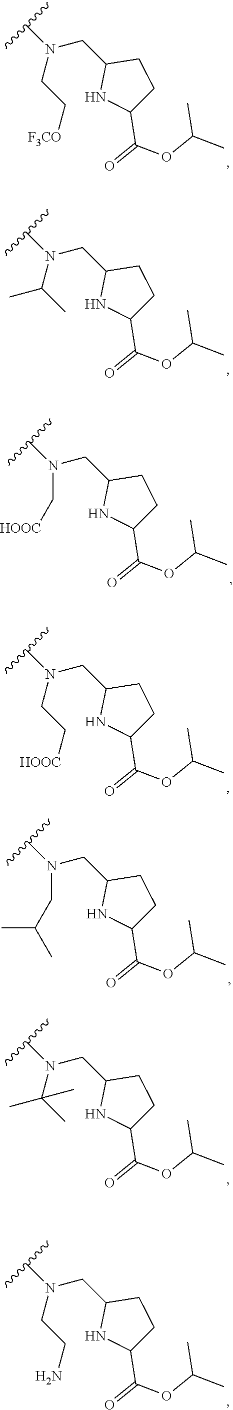

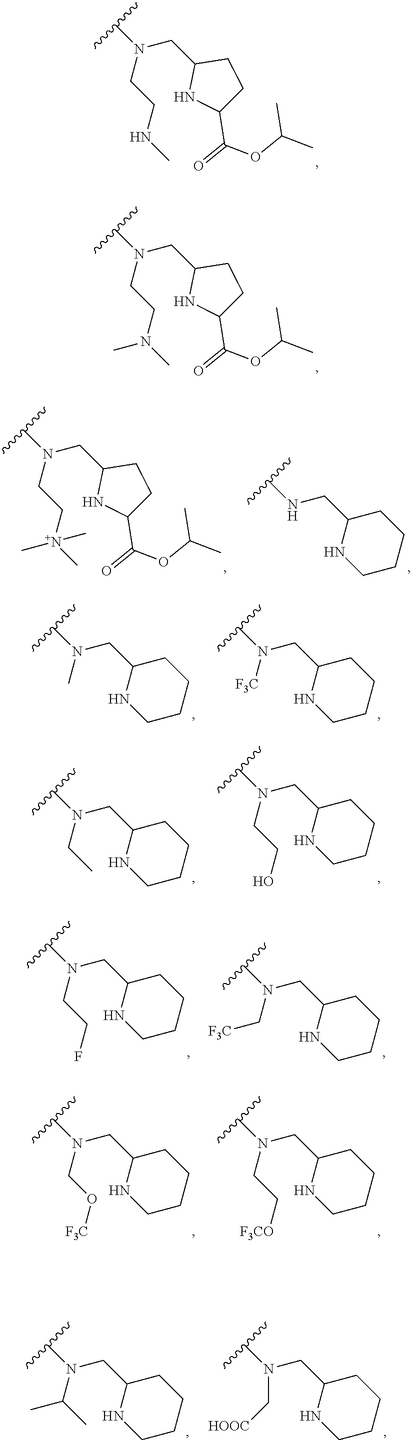

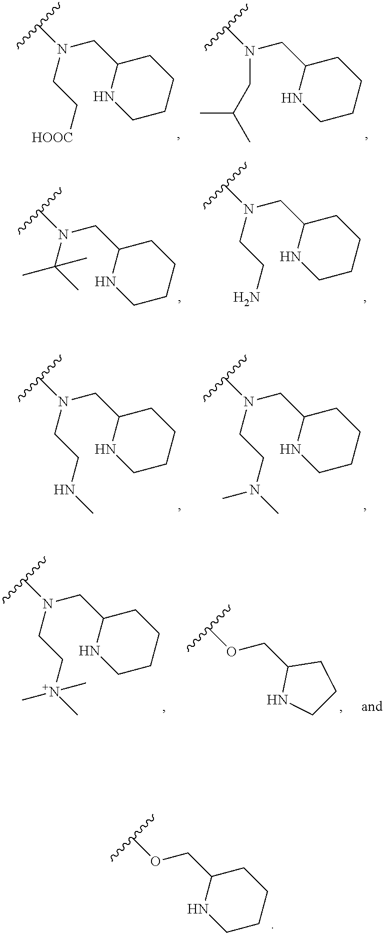

In some embodiments, the ring carbon atom in the pyrrolidine or piperidine moiety that connects to the rest of

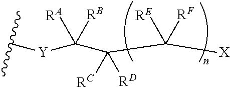

is in the S configuration. In some embodiments, the ring carbon atom in the pyrrolidine or piperidine moiety that connects to the rest of

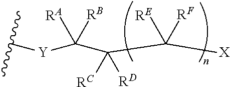

is in the R configuration.

In some embodiments, the

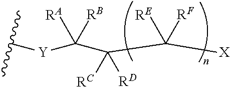

moiety is selected from:

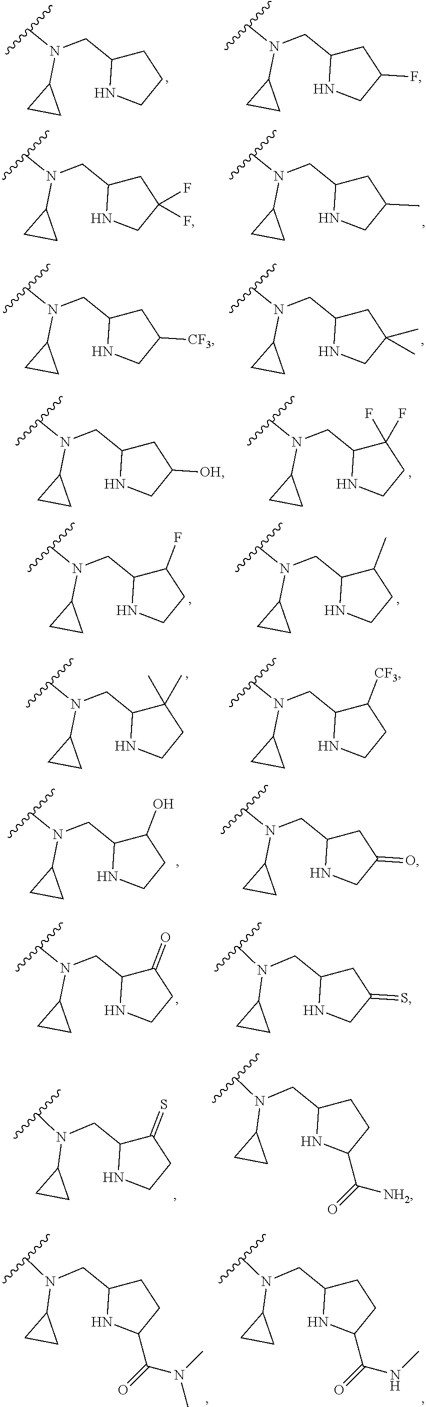

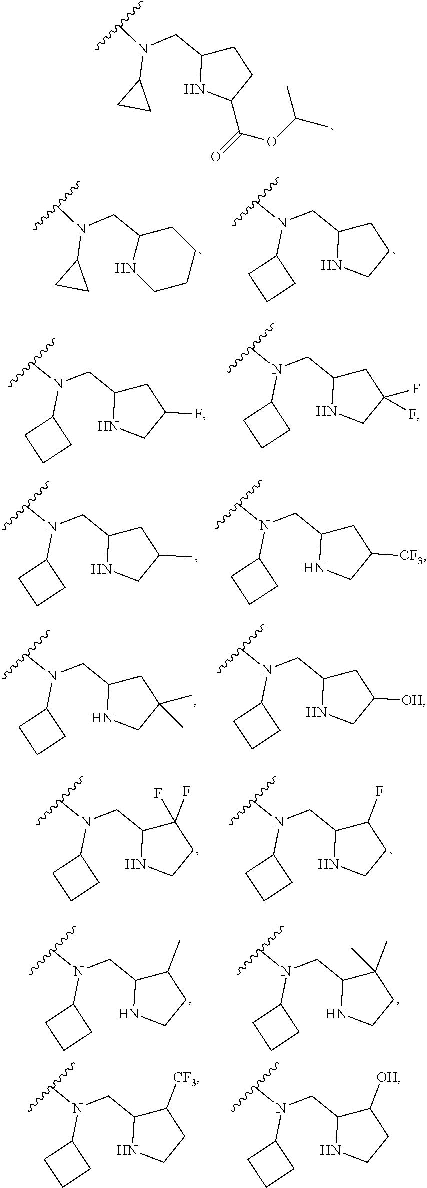

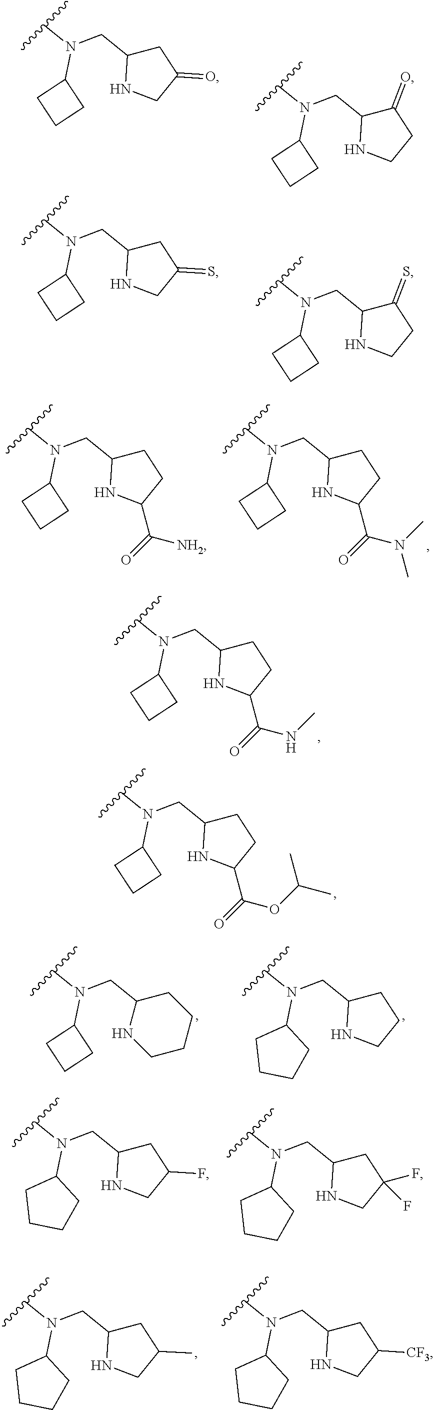

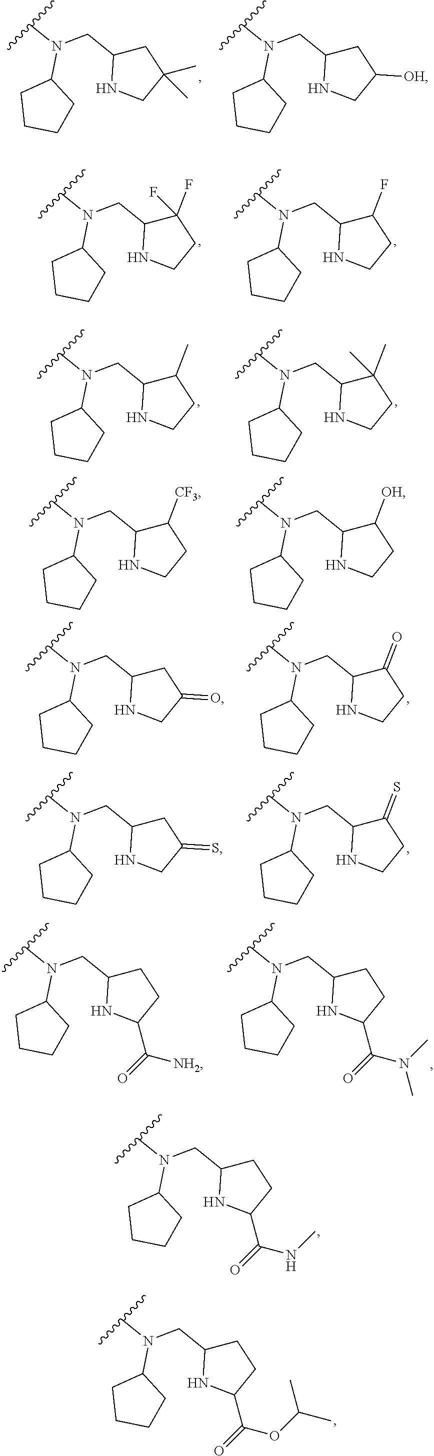

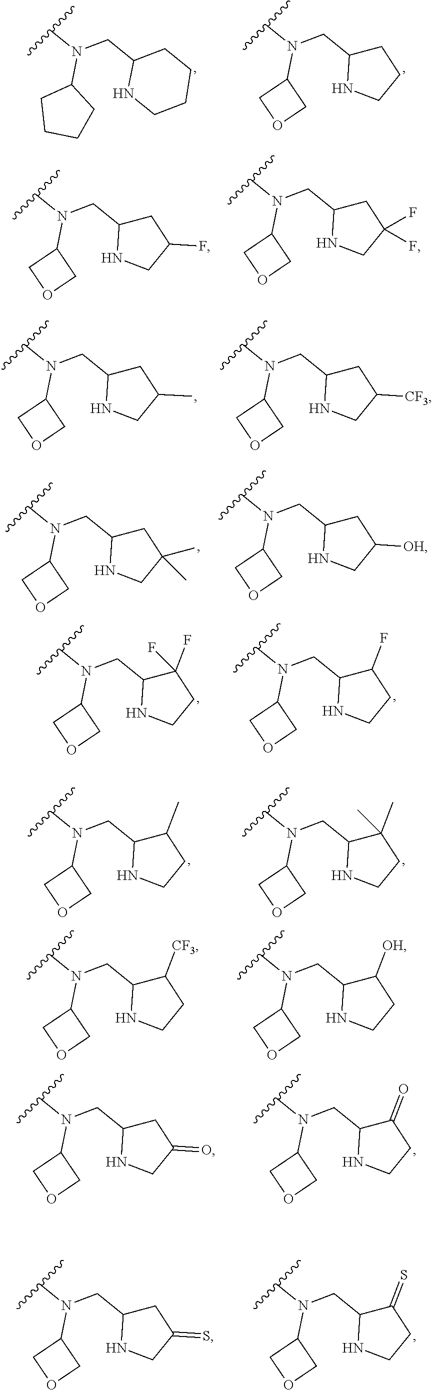

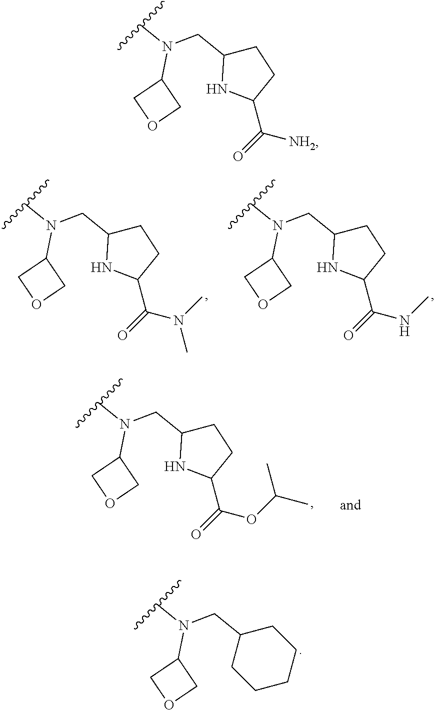 [SMILES: *N(CC1CCC(C(N)=O)N1)C1COC1 ; *N(CC1CCC(C(=O)N(C)C)N1)C1COC1 ; *N(CC1CCC(C(=O)NC)N1)C1COC1 ; *N(CC1CCC(C(=O)OC(C)C)N1)C1COC1 ; *N(CC1CCCCC1)C1COC1]

In some embodiments, the ring carbon atom in the pyrrolidine or piperidine moiety that connects to the rest of

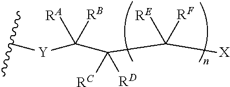

is in the S configuration. In some embodiments, the ring carbon atom in the pyrrolidine or piperidine moiety that connects to the rest of

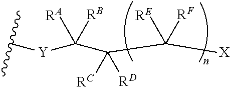

is in the R configuration.
In some embodiments, the

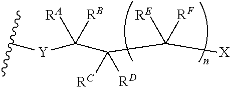

moiety is selected from:

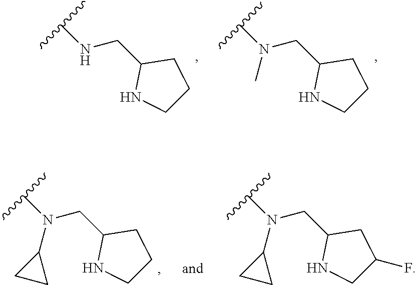

In some embodiments, the ring carbon atom in the pyrrolidine moiety that connects to the rest of

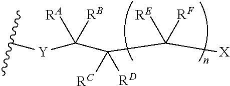

is in the S configuration. In some embodiments, the ring carbon atom in the pyrrolidine moiety that connects to the rest of

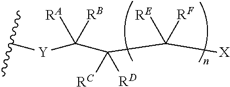

is in the R configuration.

In some embodiments, the

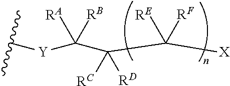

moiety is

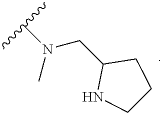 [SMILES: *N(C)CC1CCCN1]

In some embodiments, the

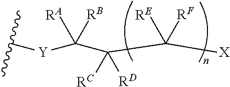

moiety is

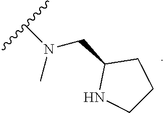 [SMILES: *N(C)C[C@H]1CCCN1]

In some embodiments, the

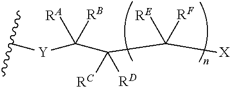

moiety is

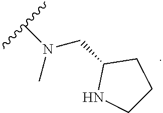 [SMILES: *N(C)C[C@@H]1CCCN1]

In some embodiments, the

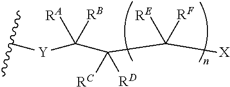

moiety is selected from:

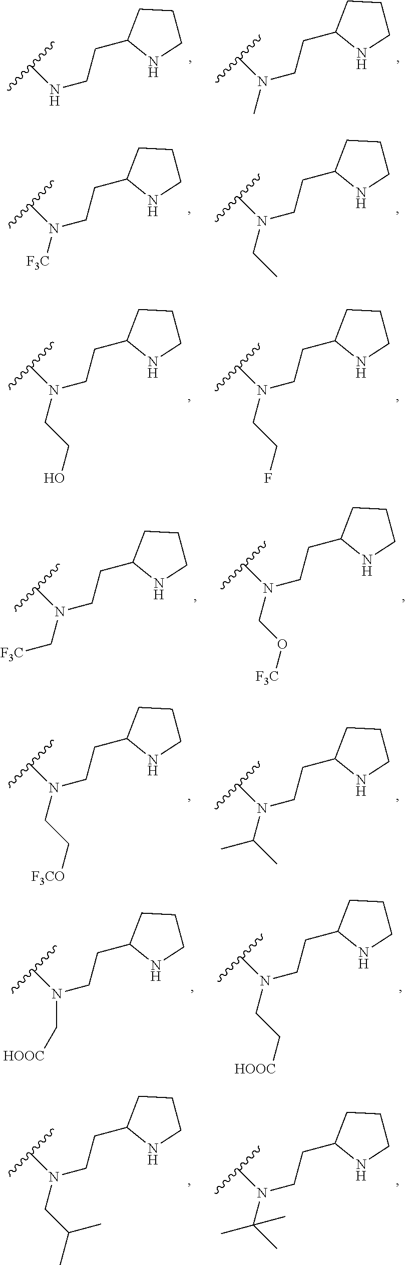

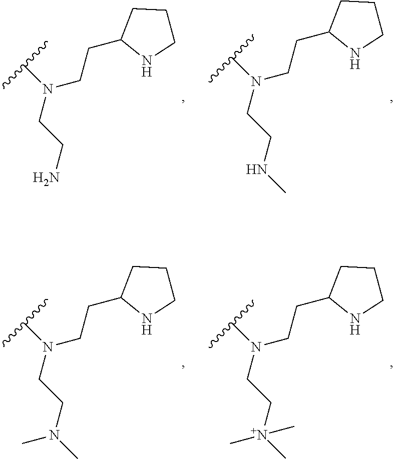 [SMILES: *N(CCN)CCC1CCCN1 ; *N(CCNC)CCC1CCCN1 ; *N(CCC1CCCN1)CCN(C)C ; *N(CCC1CCCN1)CC[N+](C)(C)C]

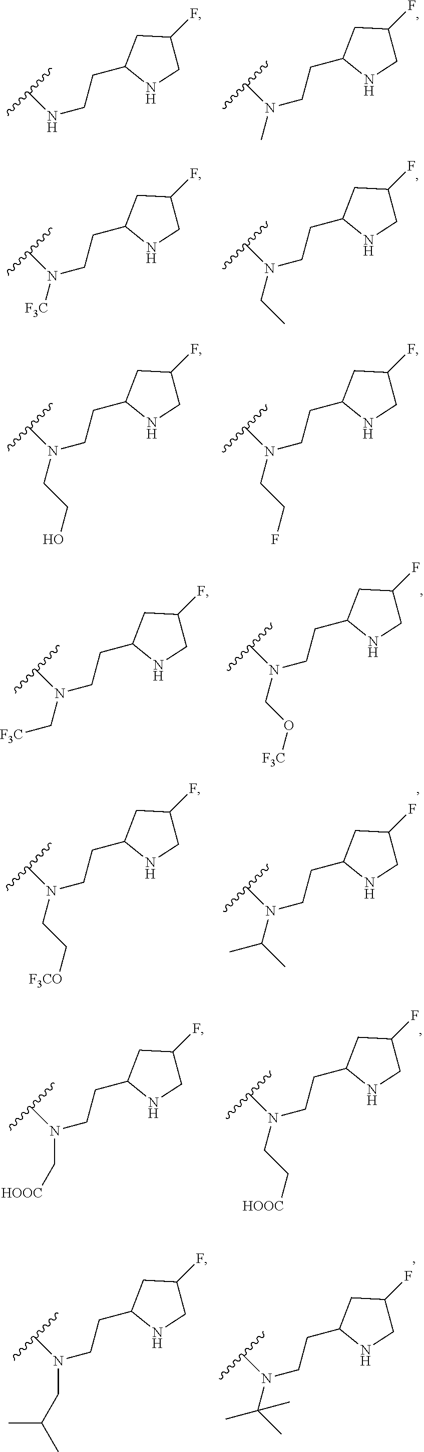

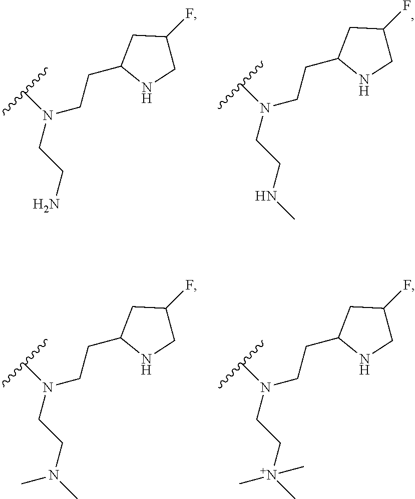 [SMILES: *N(CCN)CCC1CC(F)CN1 ; *N(CCNC)CCC1CC(F)CN1 ; *N(CCC1CC(F)CN1)CCN(C)C ; *N(CCC1CC(F)CN1)CC[N+](C)(C)C]

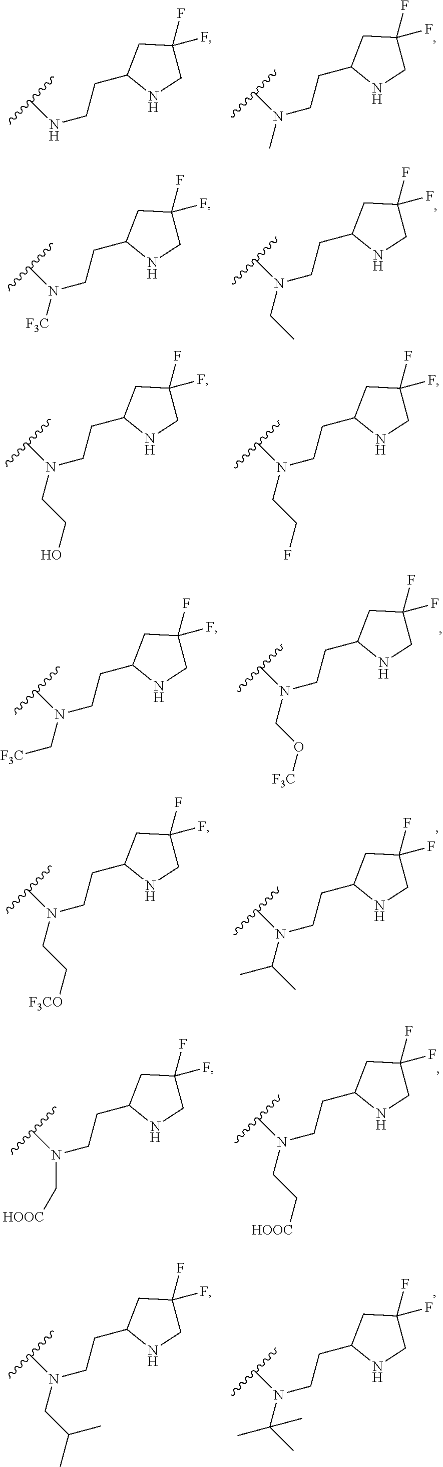

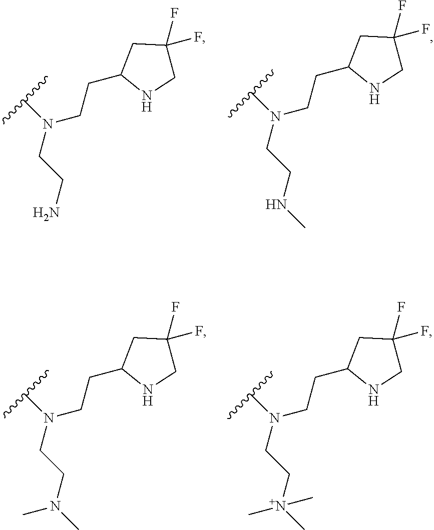 [SMILES: *N(CCN)CCC1CC(F)(F)CN1 ; *N(CCNC)CCC1CC(F)(F)CN1 ; *N(CCC1CC(F)(F)CN1)CCN(C)C ; *N(CCC1CC(F)(F)CN1)CC[N+](C)(C)C]

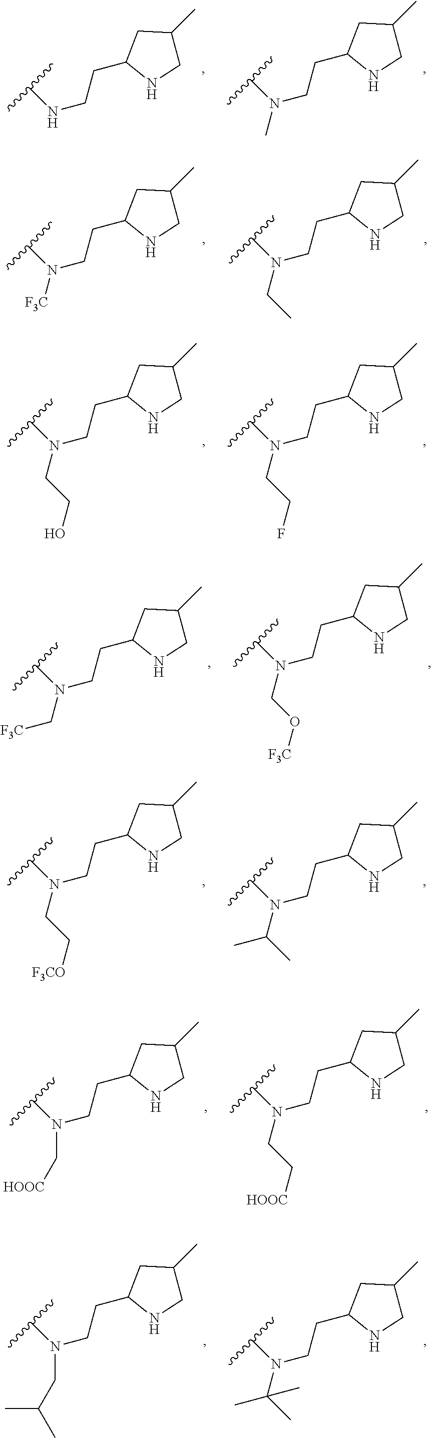

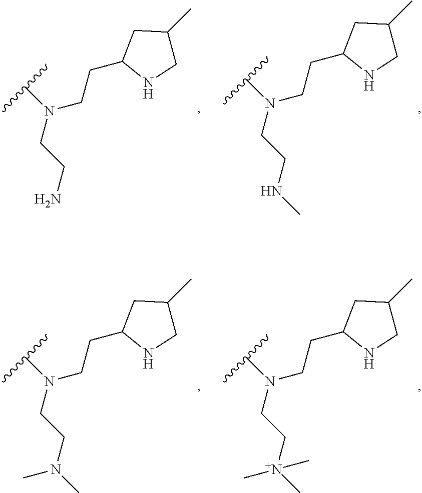

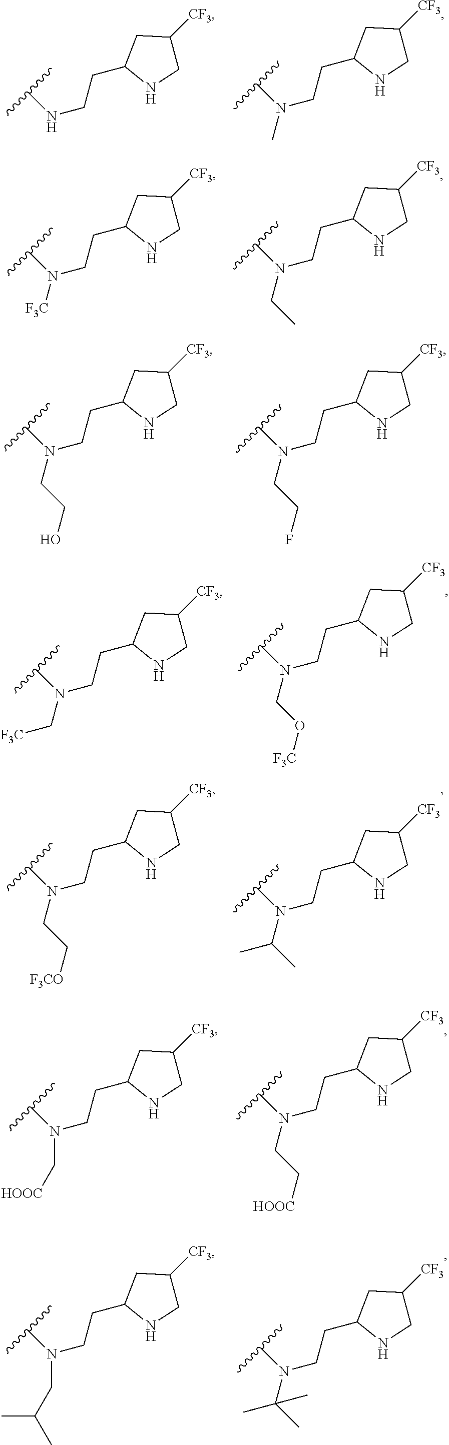

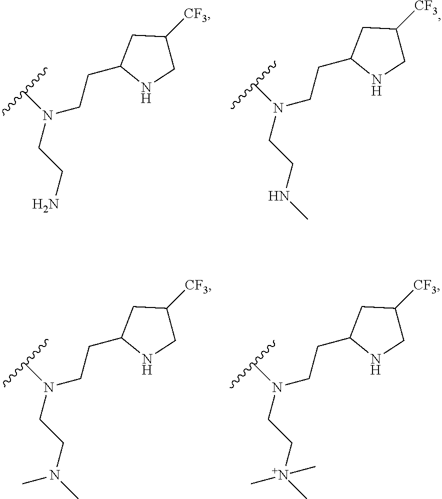 [SMILES: *N(CCN)CCC1CC(C(F)(F)F)CN1 ; *N(CCNC)CCC1CC(C(F)(F)F)CN1 ; *N(CCC1CC(C(F)(F)F)CN1)CCN(C)C ; *N(CCC1CC(C(F)(F)F)CN1)CC[N+](C)(C)C]

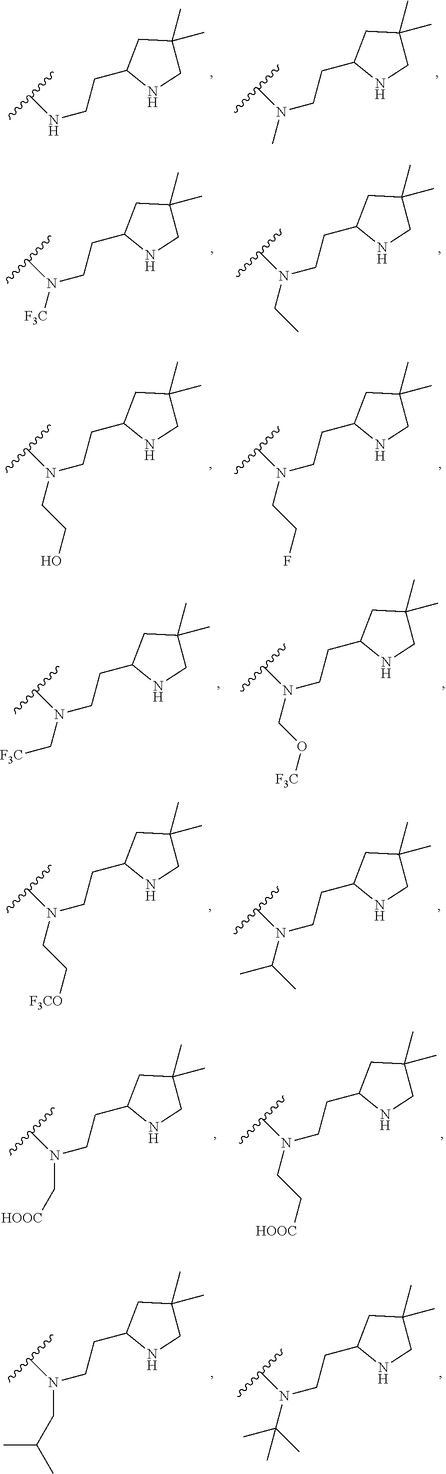

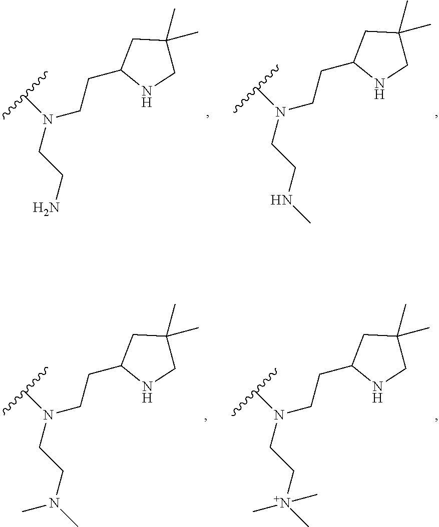

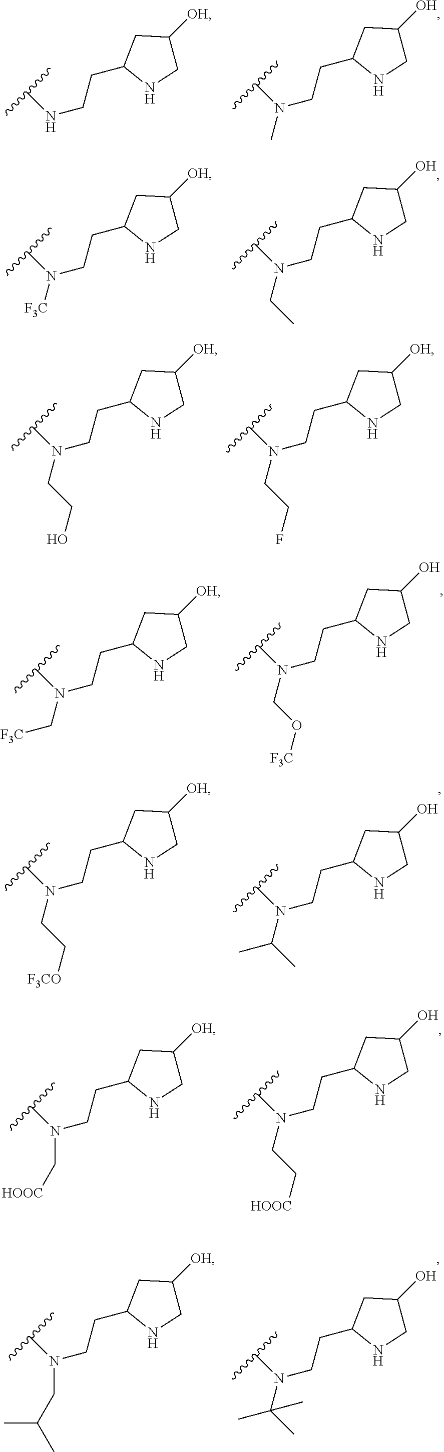

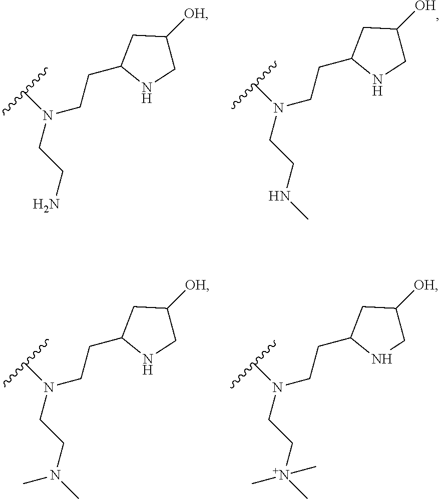 [SMILES: *N(CCN)CCC1CC(O)CN1 ; *N(CCNC)CCC1CC(O)CN1 ; *N(CCC1CC(O)CN1)CCN(C)C ; *N(CCC1CC(O)CN1)CC[N+](C)(C)C]

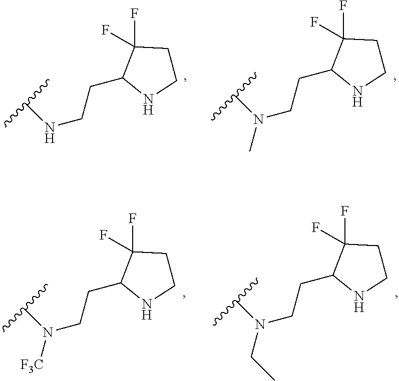 [SMILES: *NCCC1NCCC1(F)F ; *N(C)CCC1NCCC1(F)F ; *N(CCC1NCCC1(F)F)C(F)(F)F ; *N(CC)CCC1NCCC1(F)F]

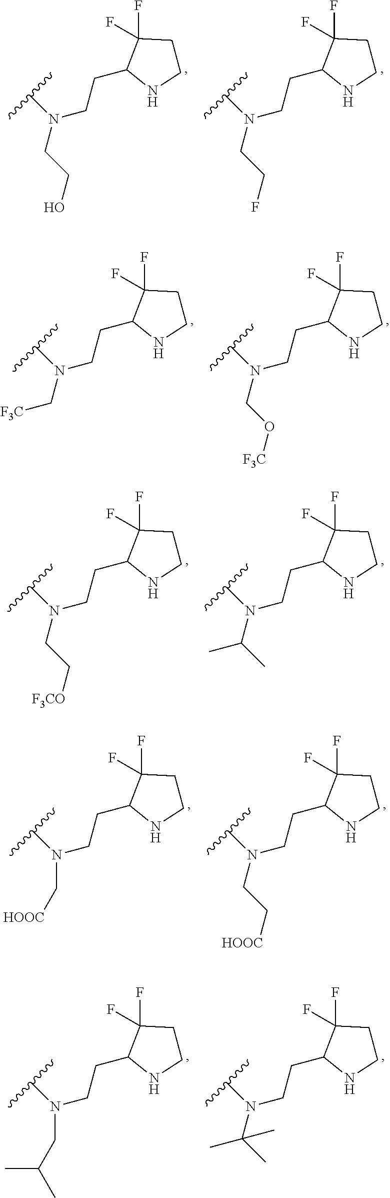

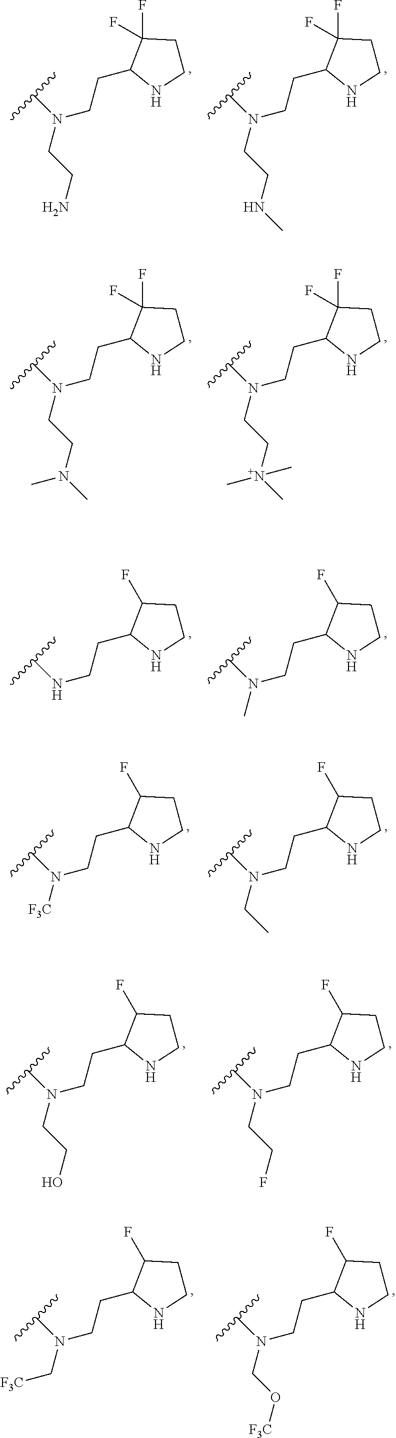

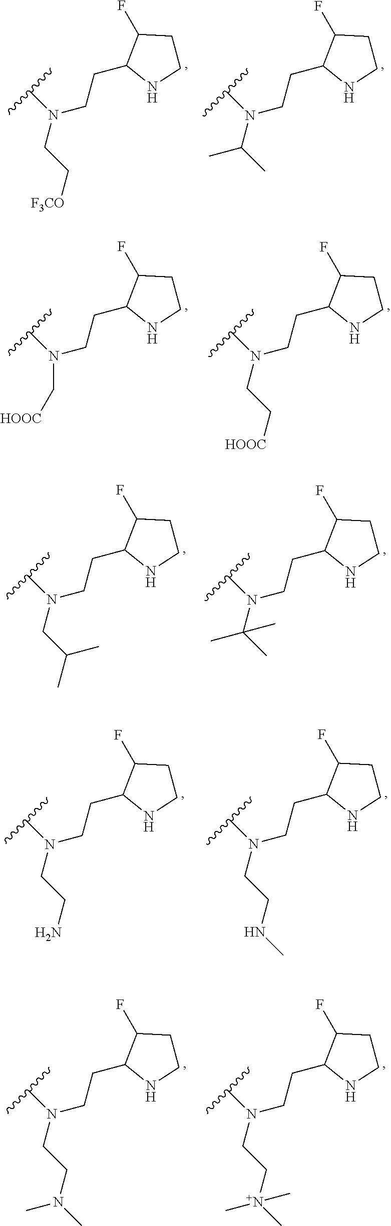

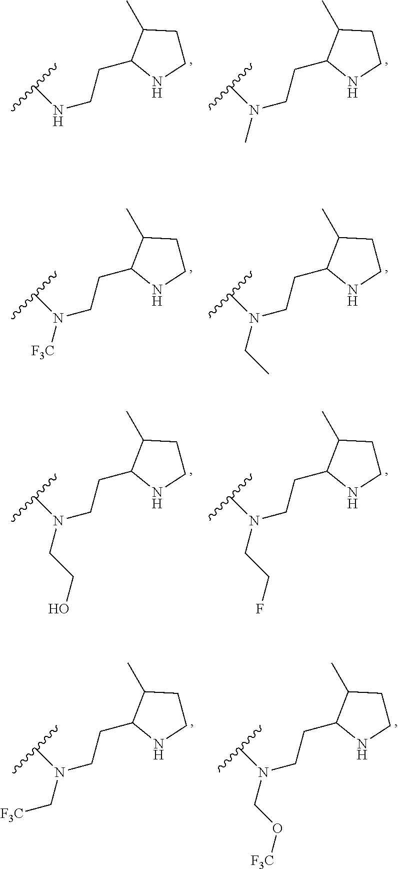

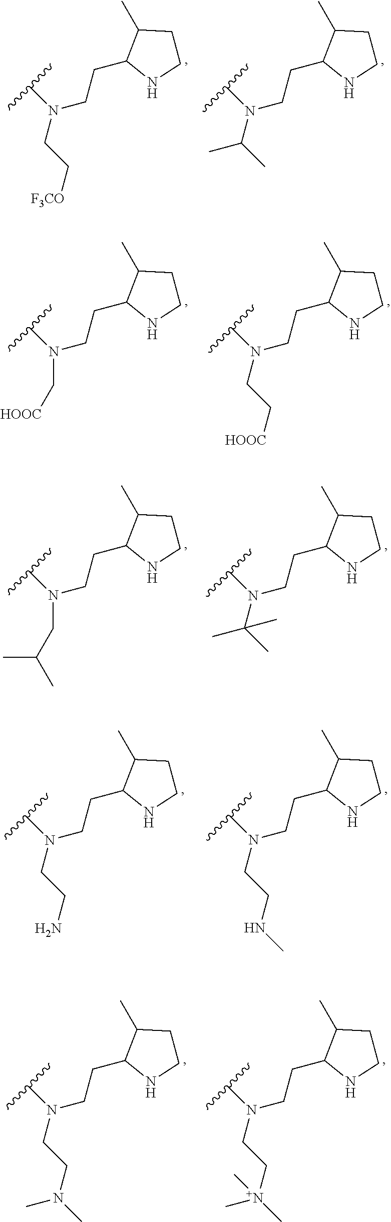

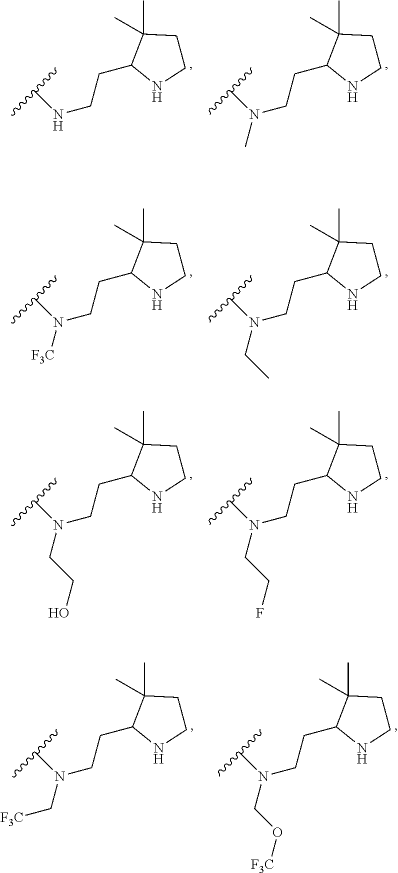

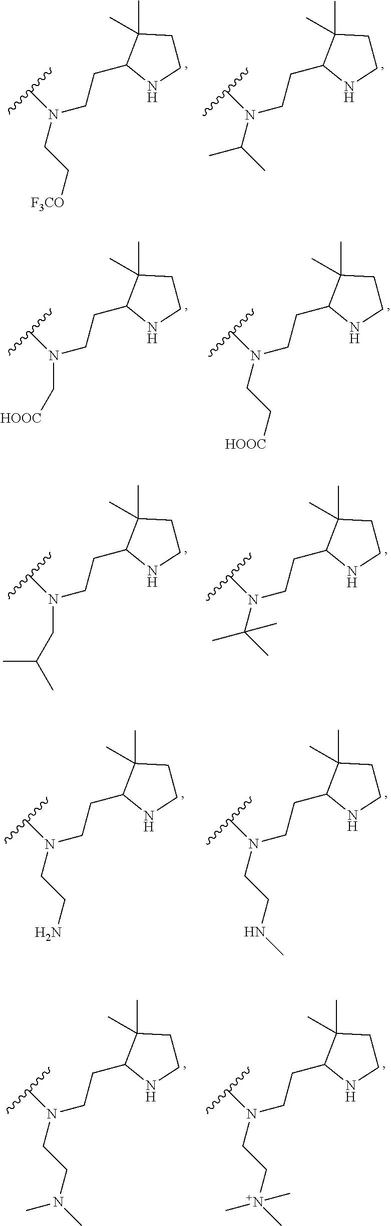

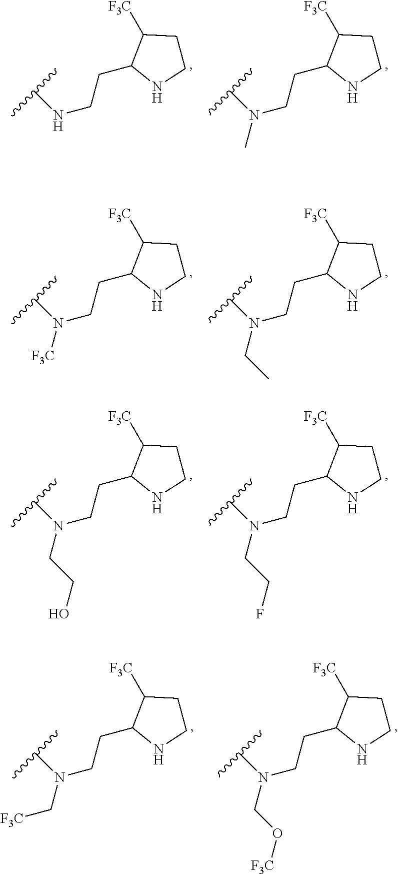

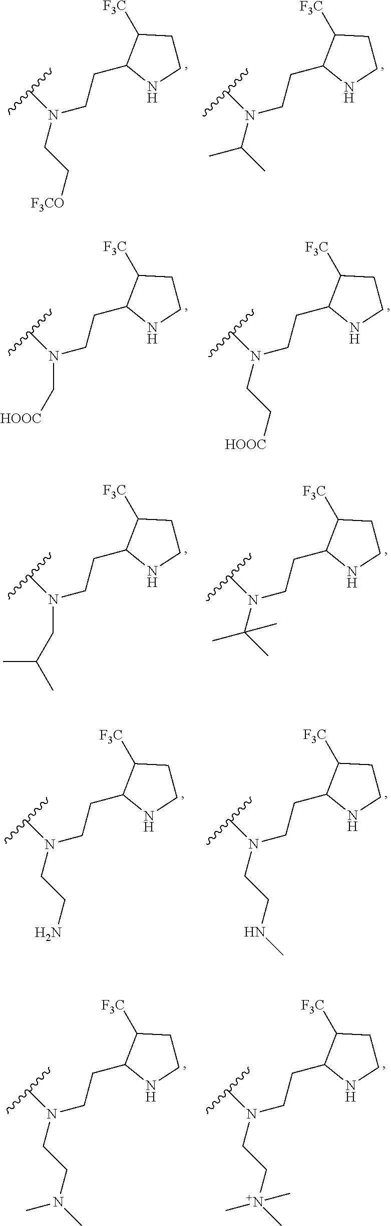

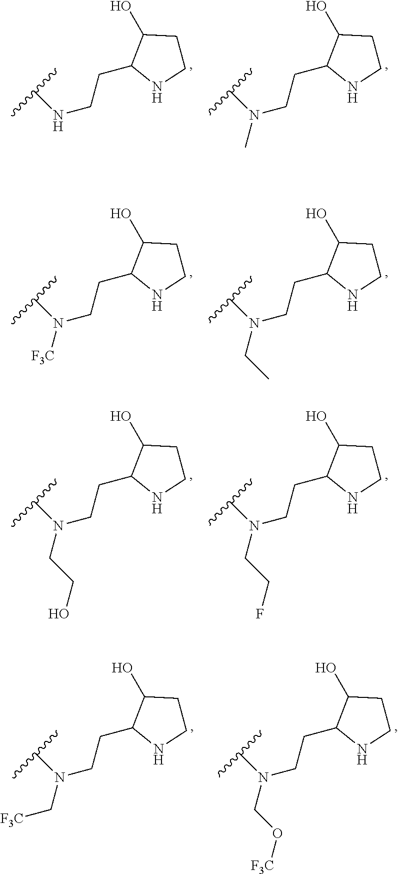

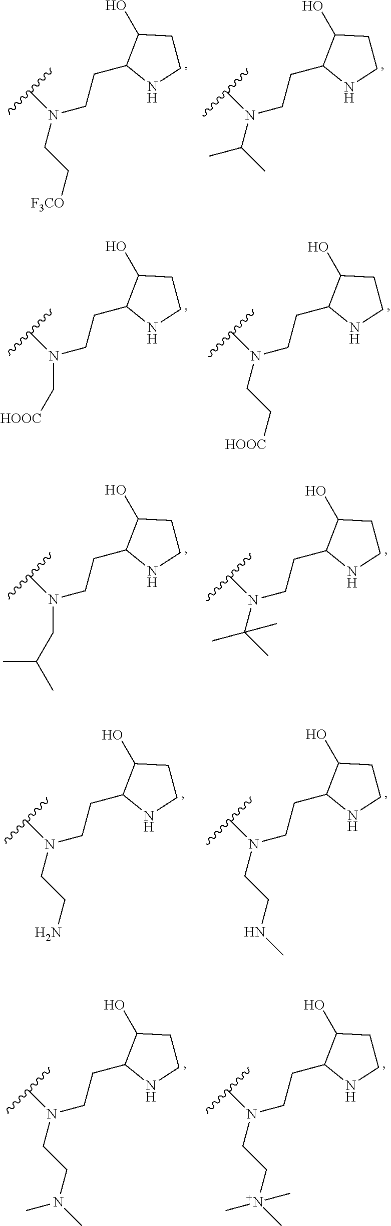

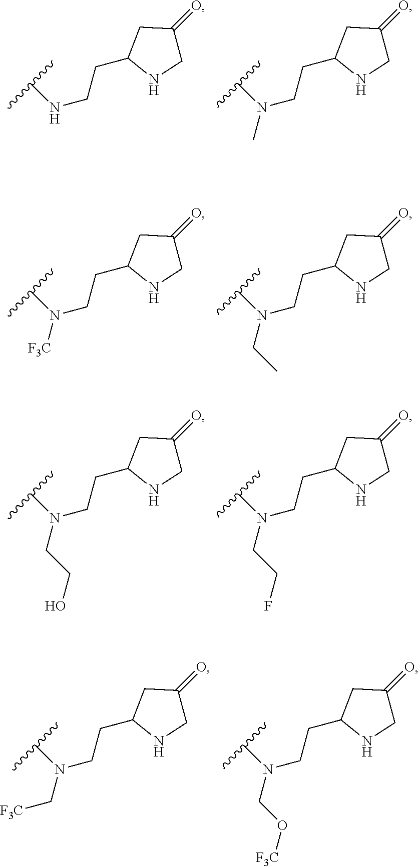

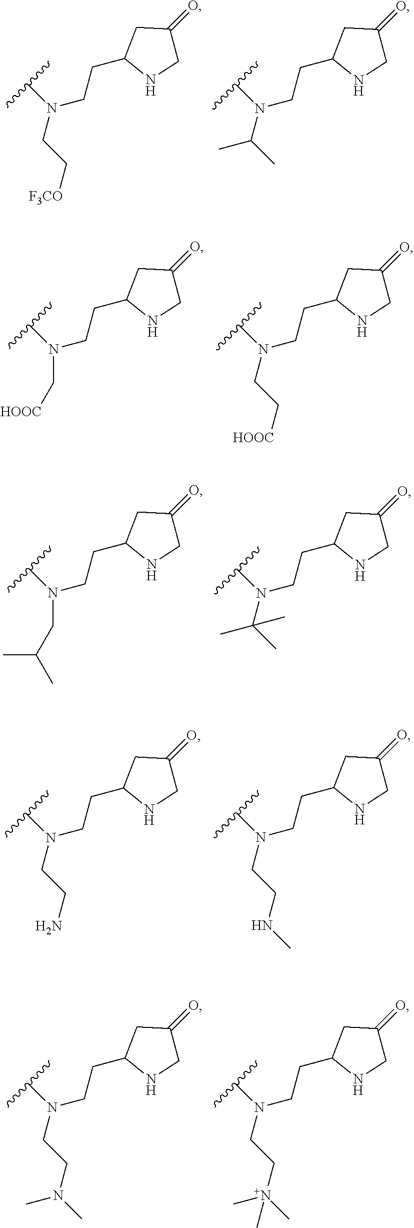

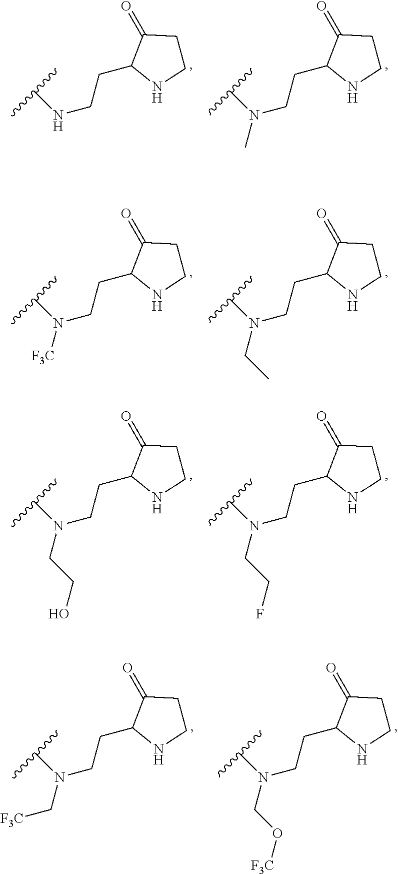

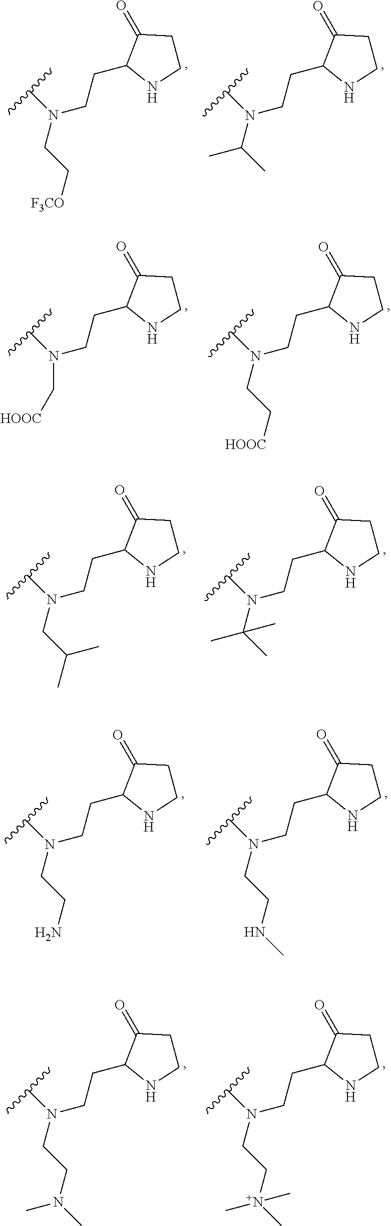

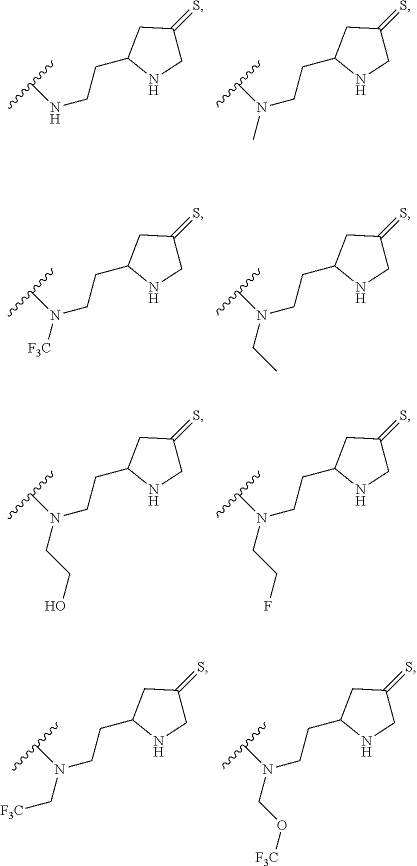

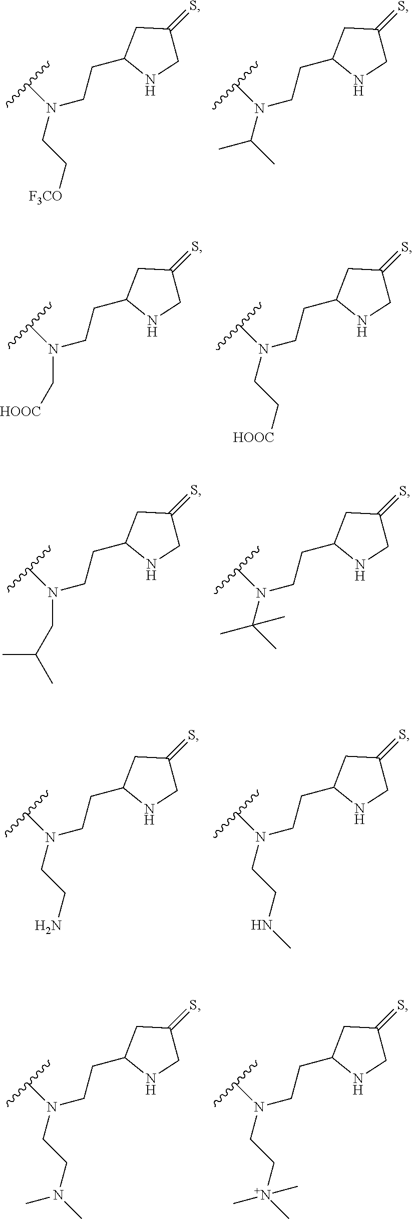

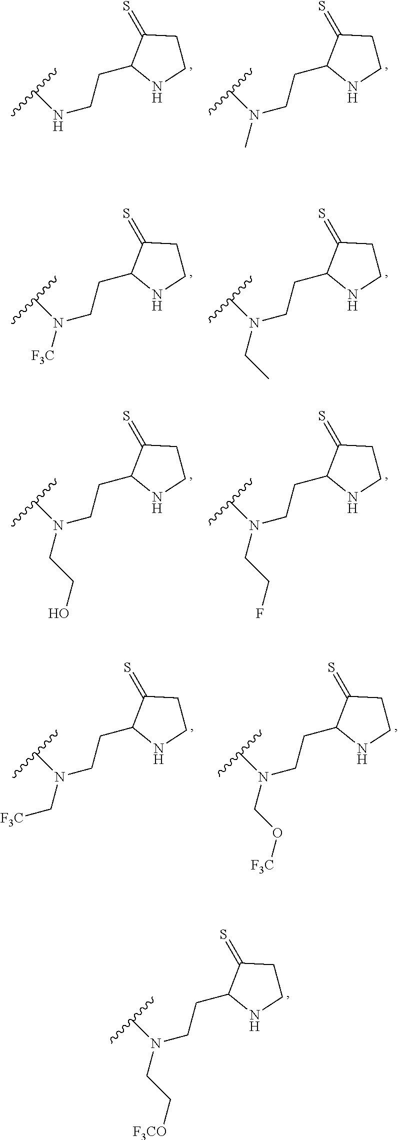

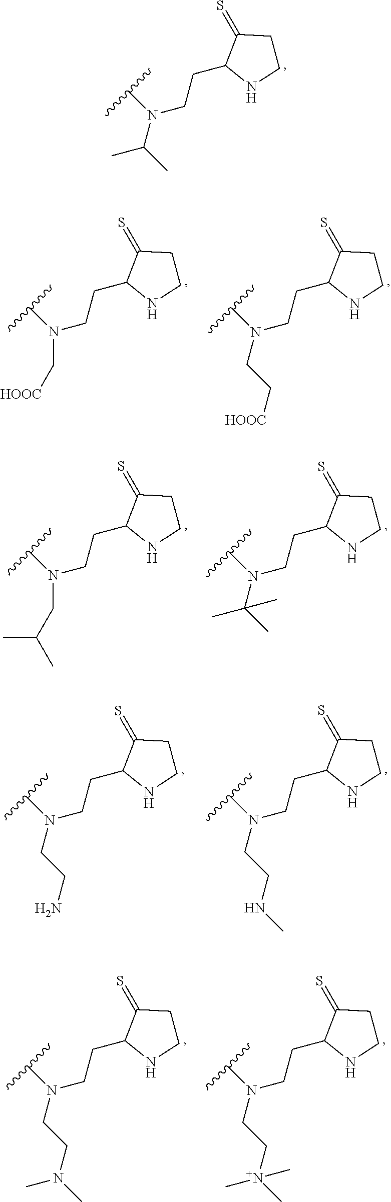

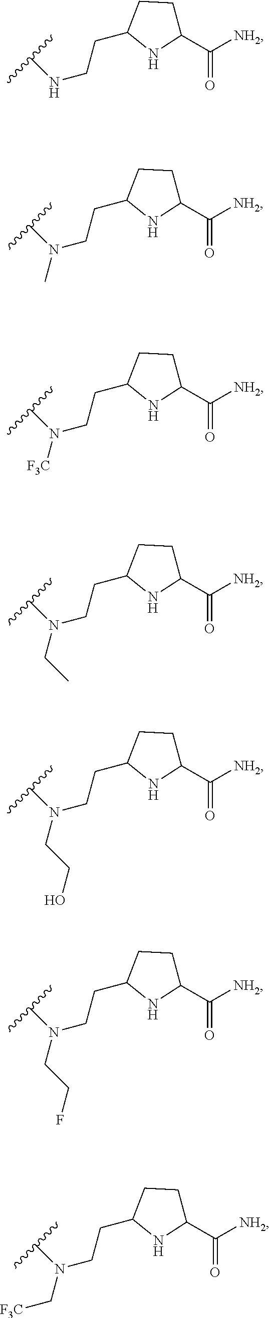

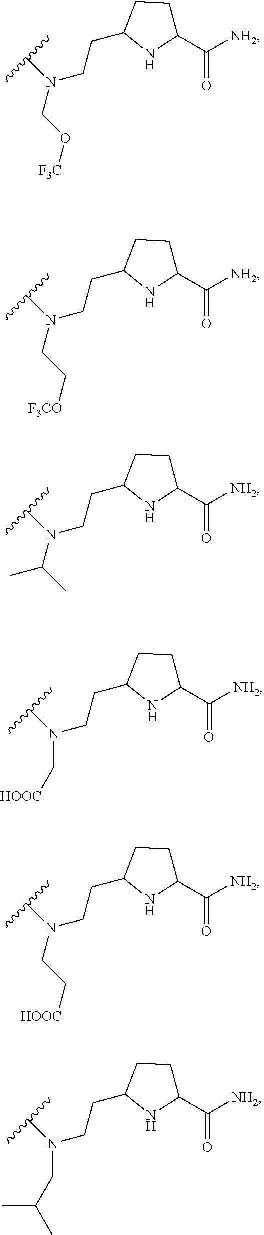 [SMILES: *N(CCC1CCC(C(N)=O)N1)COC(F)(F)F ; *N(CCOC(F)(F)F)CCC1CCC(C(N)=O)N1 ; *N(CCC1CCC(C(N)=O)N1)C(C)C ; *N(CCC1CCC(C(N)=O)N1)CC(=O)O ; *N(CCC(=O)O)CCC1CCC(C(N)=O)N1 ; *N(CCC1CCC(C(N)=O)N1)CC(C)C]

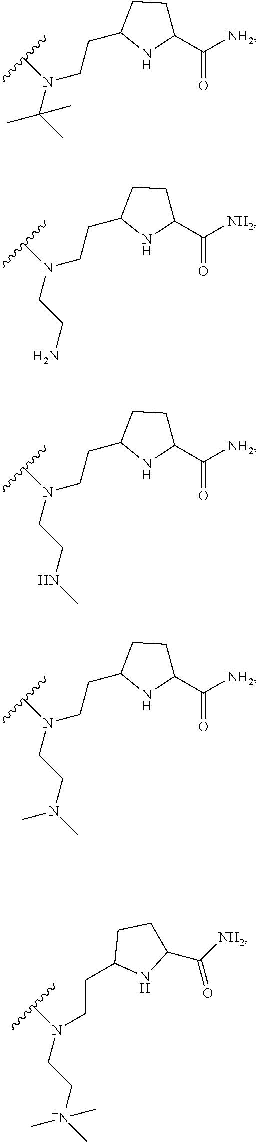 [SMILES: *N(CCC1CCC(C(N)=O)N1)C(C)(C)C ; *N(CCN)CCC1CCC(C(N)=O)N1 ; *N(CCNC)CCC1CCC(C(N)=O)N1 ; *N(CCC1CCC(C(N)=O)N1)CCN(C)C ; *N(CCC1CCC(C(N)=O)N1)CC[N+](C)(C)C]

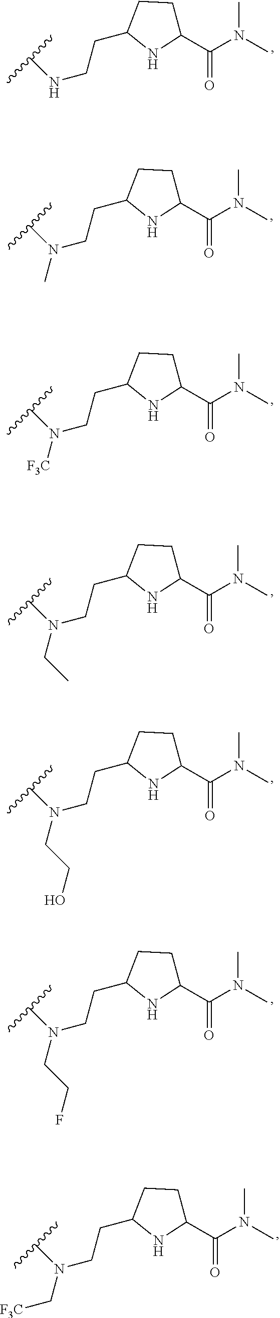

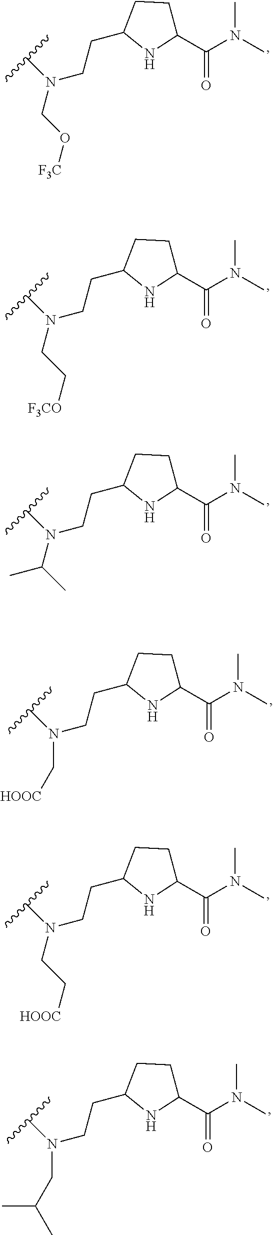

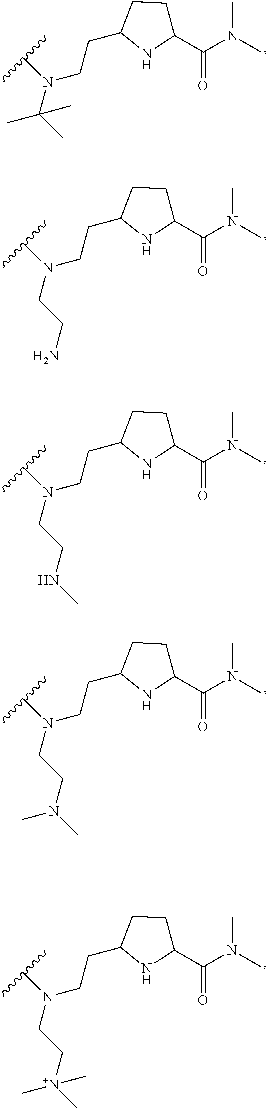

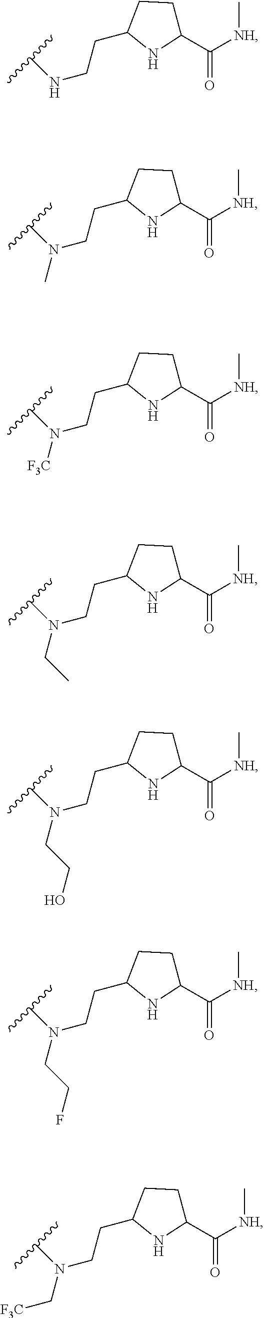

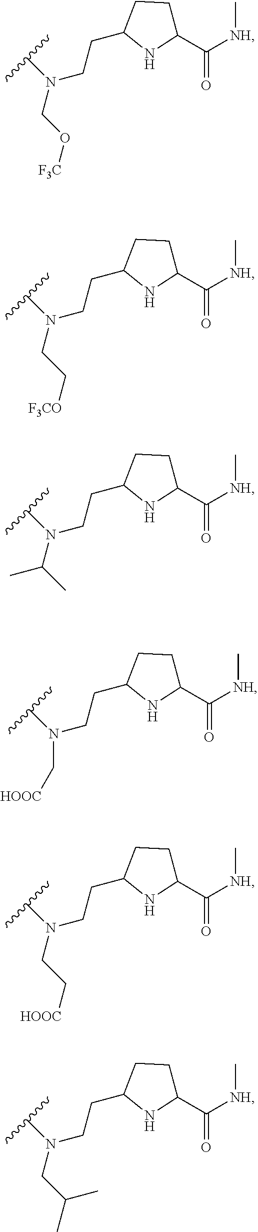

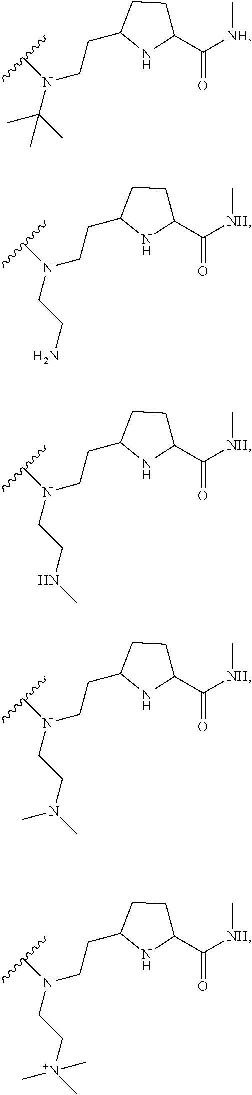

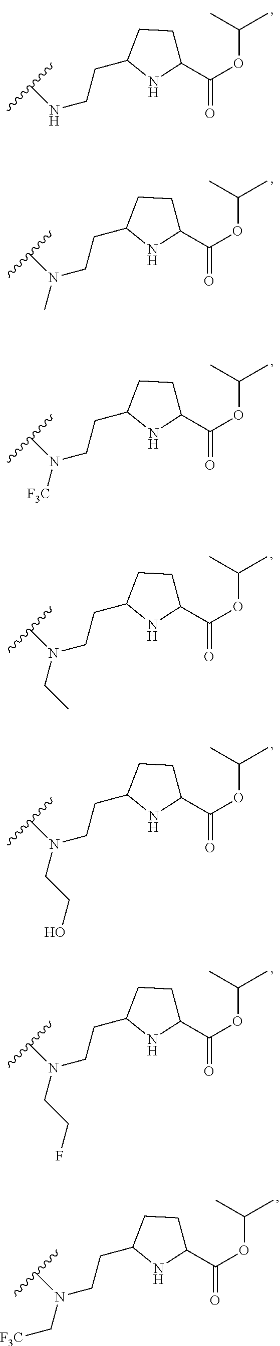

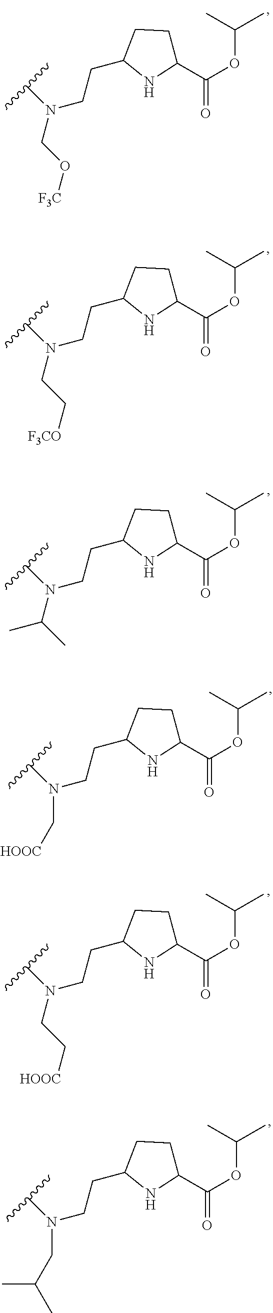

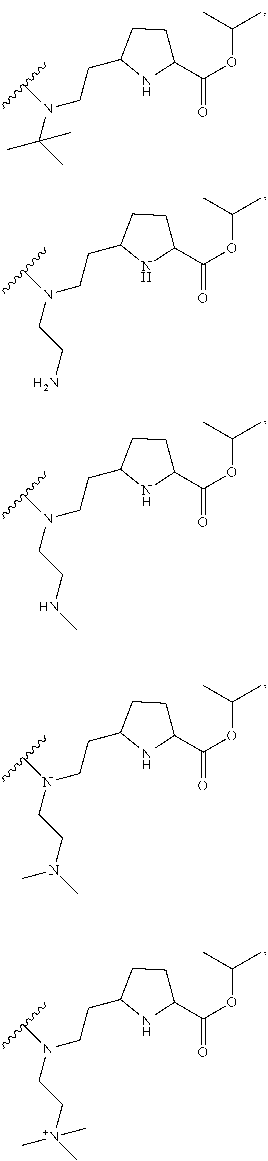

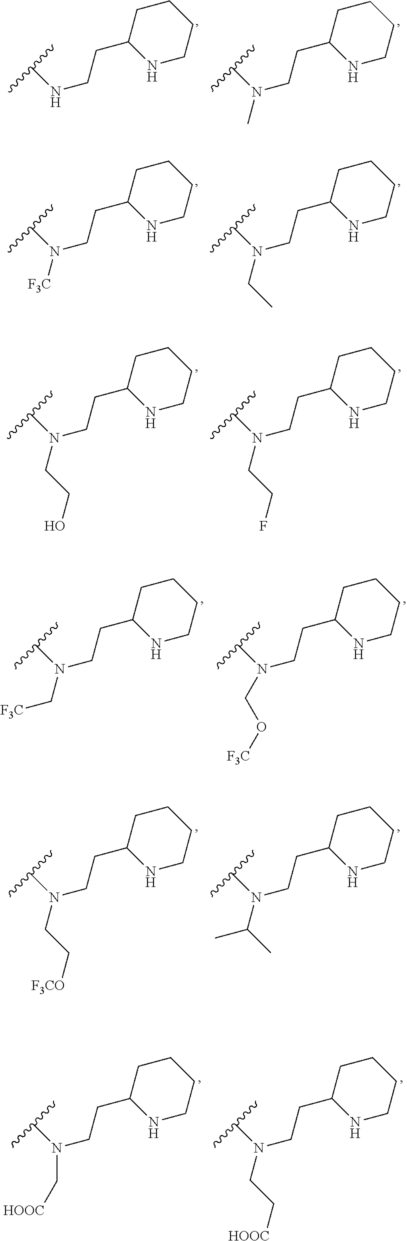

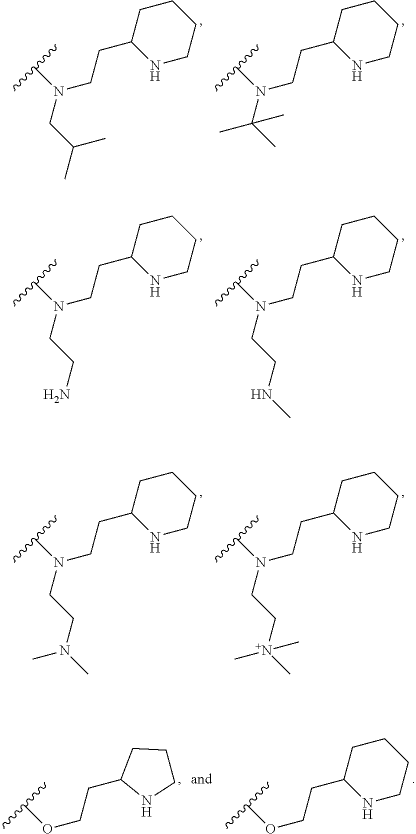

In some embodiments, the ring carbon atom in the pyrrolidine or piperidine moiety that connects to the rest of

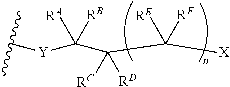

is in the S configuration. In some embodiments, the ring carbon atom in the pyrrolidine or piperidine moiety that connects to the rest of

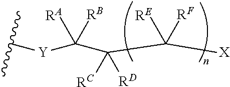

is in the R configuration.

In some embodiments, the

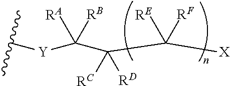

moiety is selected from:

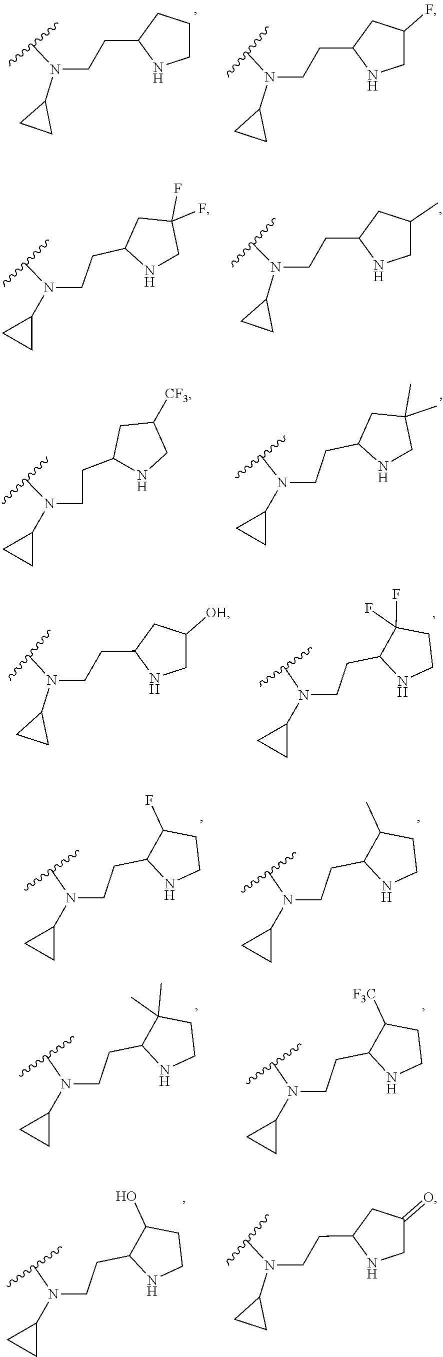

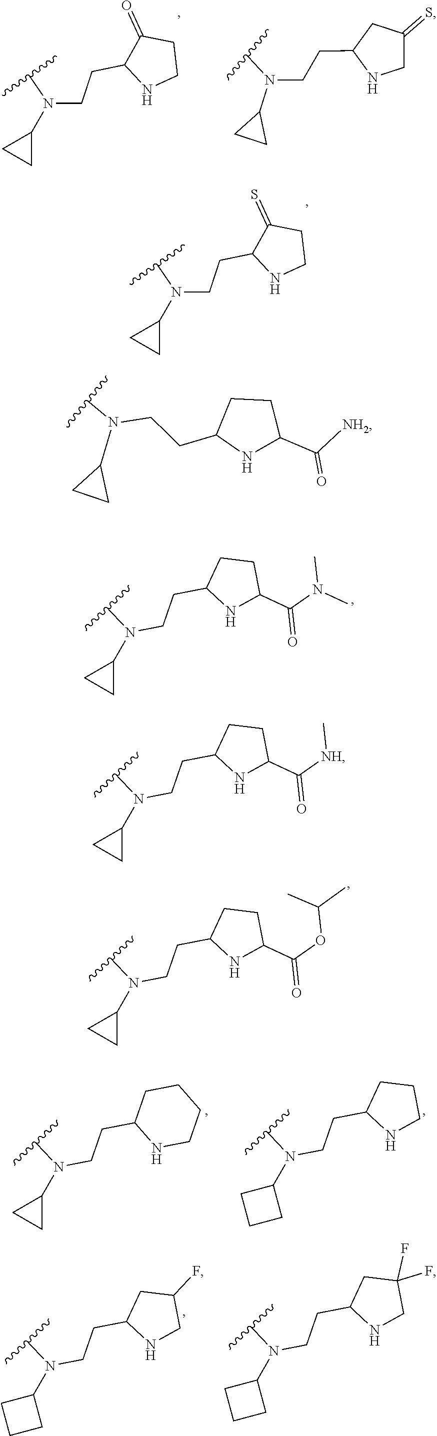

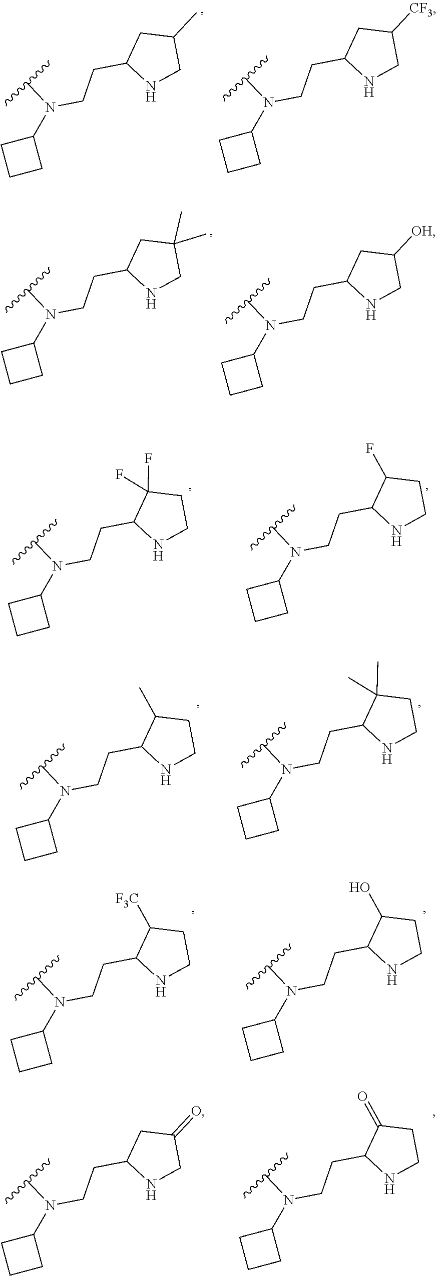

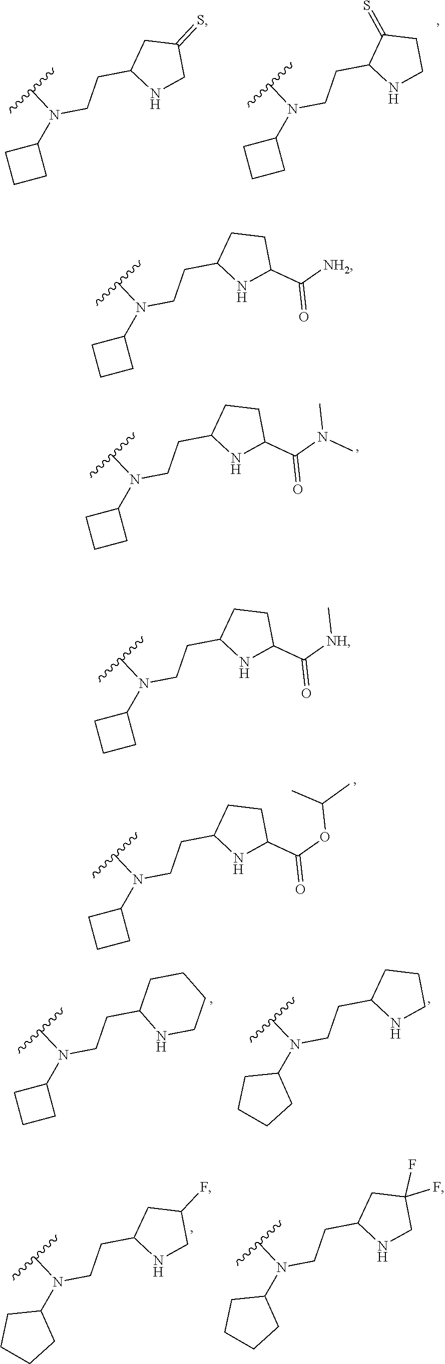

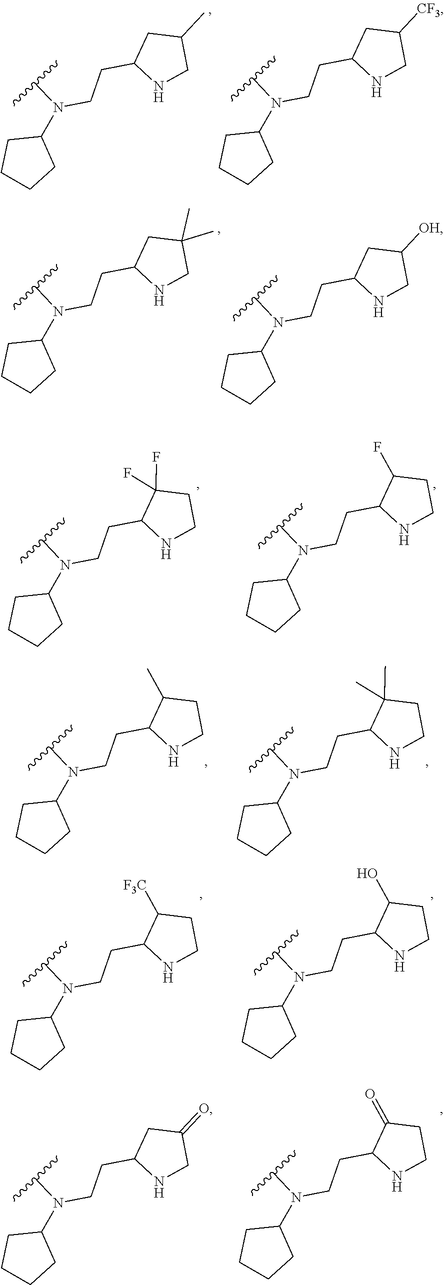

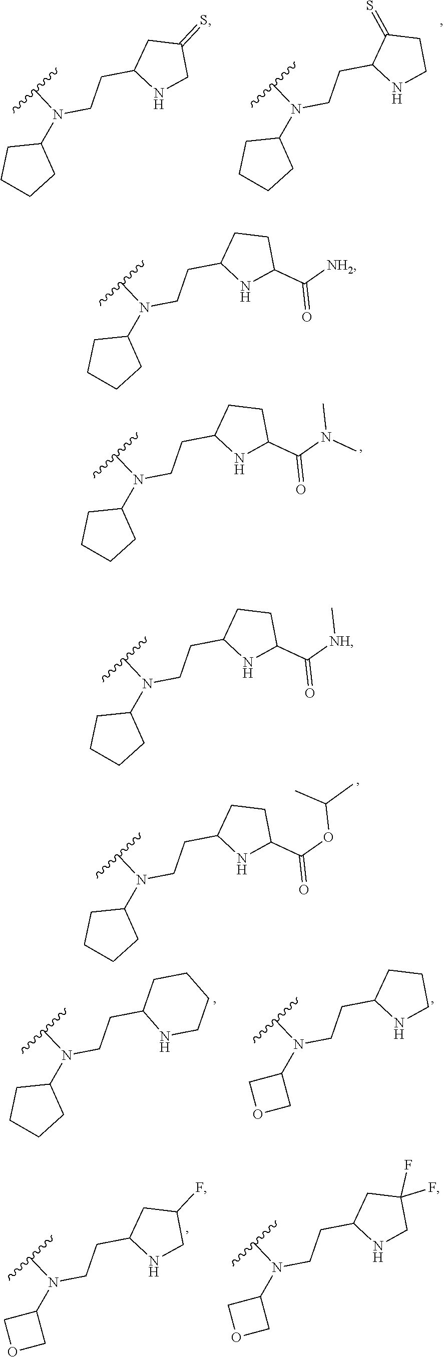

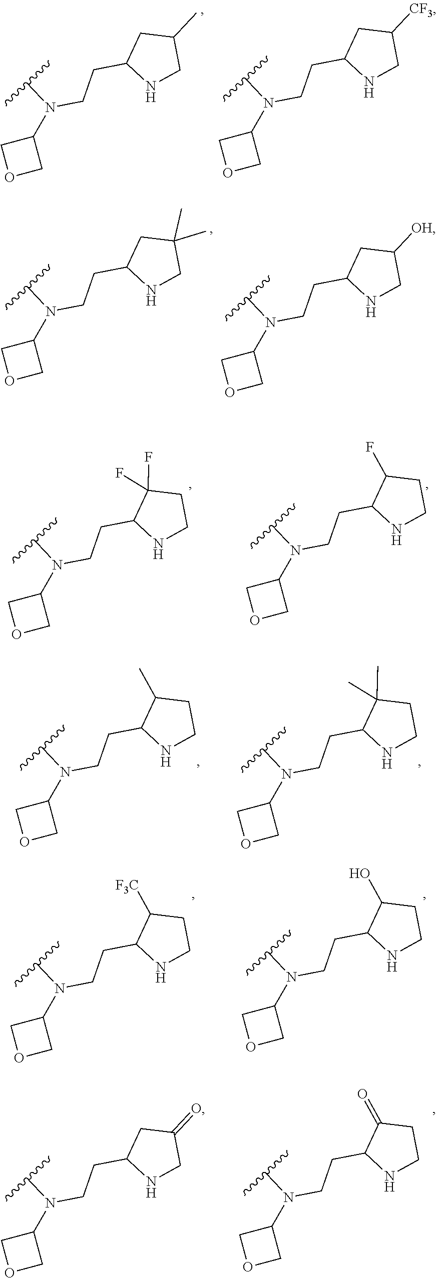

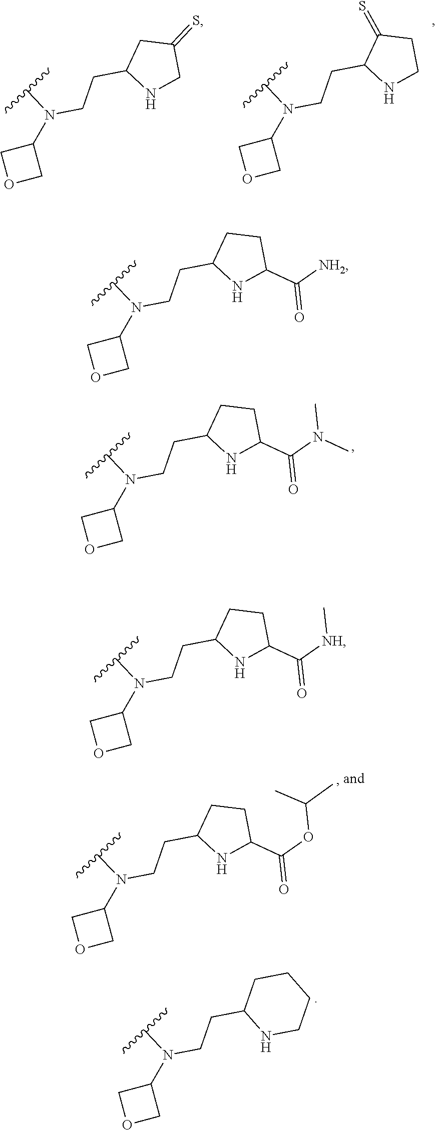

In some embodiments, the ring carbon atom in the pyrrolidine or piperidine moiety that connects to the rest of

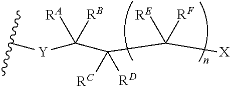

is in the S configuration. In some embodiments, the ring carbon atom in the pyrrolidine or piperidine moiety that connects to the rest of

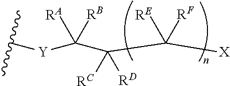

is in the R configuration.

In some embodiments, the

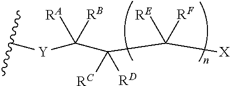

moiety is selected from:

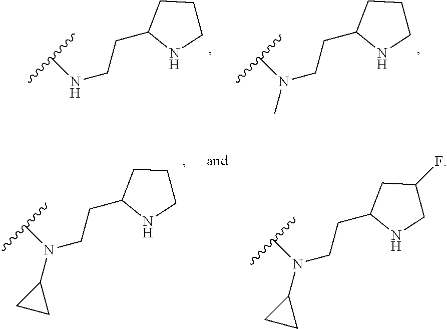

In some embodiments, the ring carbon atom in the pyrrolidine moiety that connects to the rest of

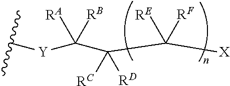

is in the S configuration. In some embodiments, the ring carbon atom in the pyrrolidine moiety that connects to the rest of

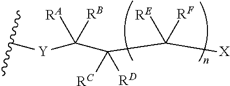

is in the R configuration.

In some embodiments the

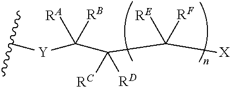

moiety is selected from:

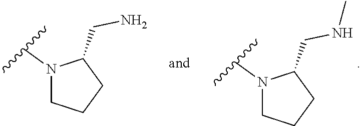

In some embodiments, the foregoing exemplified prodrugs are in a non-salt form as shown in the structures. In some embodiments, the prodrugs are in a salt form. In some embodiments, the prodrugs are in an HCl salt form.

### 4. Properties

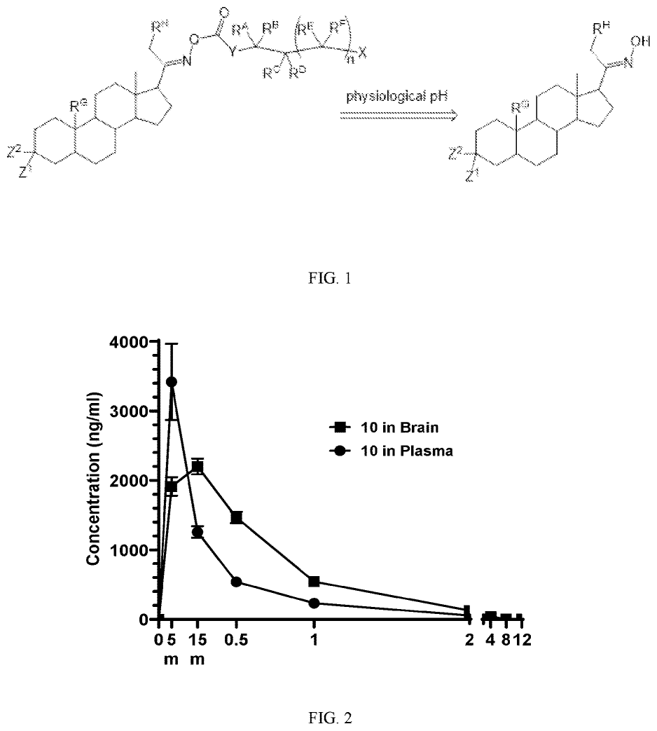

In general, the prodrugs disclosed in this section are highly soluble in an aqueous medium. They may be capable of self-immolative cleavage in response to environmental pH changes, releasing the parent neurosteroid C20-oxime (see FIG. 1 for an exemplary illustration).

In some embodiments, the prodrugs are stable in an acidic (pH<7) aqueous medium but exhibit a wide range of release kinetics in human plasma.

In some embodiments, the prodrugs have an aqueous stability, t_{1/2}, at pH 4.0 of at least 90 days, at least 60 days, or at least 30 days. In some embodiments, the prodrugs have an aqueous stability, t_{1/2}, at pH 4.0 of between 30 days and a year. In some embodiments, the prodrugs have an aqueous stability, t_{1/2}, at pH 4.0 of between 60 days and a year. In some embodiments, the prodrugs have an aqueous stability, t_{1/2}, at pH 4.0 of between 90 days and a year. In some embodiments, the prodrugs have an aqueous stability, t_{1/2}, at pH 4.0 of between 30 days and 180 days. In some embodiments, the prodrugs have an aqueous stability, t_{1/2}, at pH 4.0 of between 30 days and 150 days. In some embodiments, the prodrugs have an aqueous stability, t_{1/2}, at pH 4.0 of between 30 days and 120 days.

In some embodiments, the prodrugs have an aqueous stability, t_{1/2}, at pH 5.5 of at least 90 days, at least 60 days, or at least 30 days. In some embodiments, the prodrugs have an aqueous stability, t_{1/2}, at pH 5.5 of between 30 days and a year. In some embodiments, the prodrugs have an aqueous stability, t_{1/2}, at pH 5.5 of between 60 days and a year. In some embodiments, the prodrugs have an aqueous stability, t_{1/2}, at pH 5.5 of between 90 days and a year. In some embodiments, the prodrugs have an aqueous stability, t_{1/2}, at pH 5.5 of between 30 days and 180 days. In some embodiments, the prodrugs have an aqueous stability, t_{1/2}, at pH 5.5 of between 30 days and 150 days. In some embodiments, the prodrugs have an aqueous stability, t_{1/2}, at pH 5.5 of between 30 days and 120 days.

In some embodiments, the prodrugs have a human plasma stability, t_{1/2}, of at most 24 hours, at most 12 hours, at most six hours, at most two hours, or at most one hour. In some embodiments, the prodrugs have a human plasma stability, t_{1/2}, of between zero and six hours, between zero and five hours, between zero and four hours, between zero and three hours, between zero and two hours, between zero and one hour, or between zero and half an hour. In some embodiments, the prodrugs have a human plasma stability, t_{1/2}, of between zero and two hours. In some embodiments, the prodrugs have a human plasma stability, t_{1/2}, of between zero and one hour. In some embodiments, the prodrugs have a human plasma stability, t_{1/2}, of between zero and half an hour.

### C. Phosphate Prodrugs of the Neurosteroids

In some embodiments, the prodrugs of the neurosteroids have a structure of Formula III or a pharmaceutically acceptable salt, hydrate, or hydrated salt of Formula III,

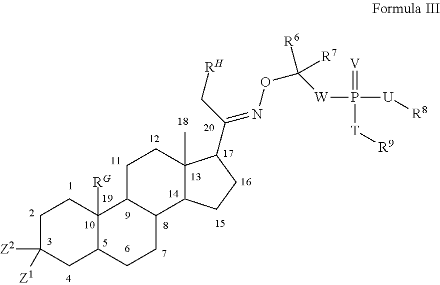

wherein R^{G}, R^{H}, Z¹, and Z² are the same as described above in Formula I;
wherein T, U, V, and W are independently O or S;
wherein R⁶ and R⁷ are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate; and
wherein R⁸ and R⁹ are independently selected from hydrogen, optionally substituted alkyl, optionally substituted heteroalkyl, optionally substituted carbocyclyl, optionally substituted heterocyclyl, optionally substituted aryl, and optionally substituted heteroaryl.

The numberings of carbon atoms in Formula III apply to all sub-formulas of Formula III.

In some embodiments, the prodrugs are in a non-salt form as shown in Formula III. In some embodiments, the prodrugs are in a salt form. In some embodiments, the prodrugs are in a Tris or bis-Tris salt form.

In general, the prodrugs described in this section have a higher solubility in an aqueous medium than their corresponding neurosteroid parent compounds. In some embodiments, the phosphate group in the prodrugs is readily cleavable by phosphatases, such as intestinal alkaline phosphatase, to release the neurosteroid parent compounds.

In some embodiments, Formula III is in the form of Formula III-1. In some embodiments, the prodrugs are in a non-salt form as shown in Formula III-1. In some embodiments, the prodrugs are in a salt form. In some embodiments, the prodrugs are in a Tris or bis-Tris salt form.

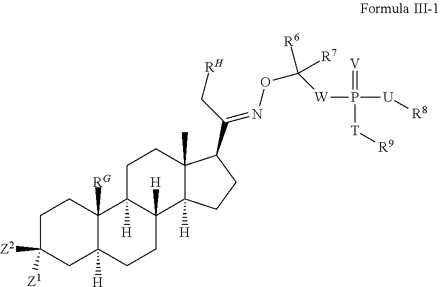

In some embodiments, Formula III is in the form of Formula III-2. In some embodiments, the prodrugs are in a non-salt form as shown in Formula III-2. In some embodiments, the prodrugs are in a salt form. In some embodiments, the prodrugs are in a Tris or bis-Tris salt form.

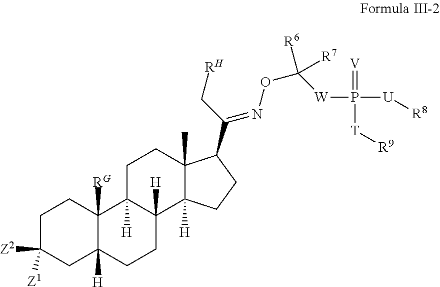

### 1. The Neurosteroid Moiety

The neurosteroid moiety

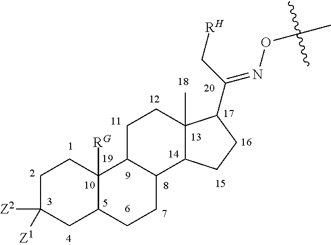

is the same as described above in Section II(A) of the Detailed Description.

In some embodiments, the neurosteroid moiety is in the following form.

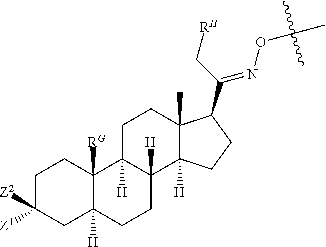

In some embodiments, the neurosteroid moiety is in the following form.

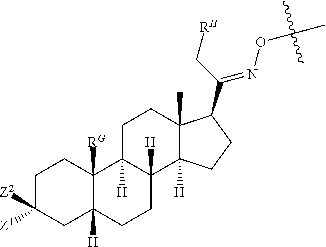

In some embodiments, R^{G} is hydrogen. In some embodiments, R^{G} is methyl.

In some embodiments, R^{H} is hydrogen. In some embodiments, R^{H} is optionally substituted nitrogen-containing 5-membered heteroaryl.

In some embodiments, R^{H} is

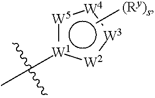

wherein W¹, W², W³, W⁴, and W⁵ are independently and individually C, CH, N, or NH, wherein at least one of W¹, W², W³, W⁴, and W⁵ is C or CH, wherein at least one of W¹, W², W³, W⁴, and W⁵ is N or NH, wherein s is 0, 1, 2, 3, or 4, wherein R⁵, at each occurrence, is independently and individually halogen, nitro, cyano, hydroxyl, formyl, carboxyl, sulfamoyl, alkyl such as methyl, haloalkyl such as CF₃, alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, arylalkyl, alkylaryl, alkyloxy, haloalkyloxy, aryloxy, haloaryloxy, alkylcarbonyl, arylcarbonyl, alkylcarbonyloxy, arylcarbonyloxy, alkyloxycarbonyl, aryloxycarbonyl, primary amino, alkylamino, alkylammonium, alkylcarbonylamino, arylcarbonylamino, carbamoyl, N-alkylcarbamoyl, alkylthio, alkylsulfinyl, alkylsulfonyl, or N-alkylsulfamoyl. It is understood by those skill in the art that when an R group is present, it replaces the hydrogen atom at the ring atom that the R⁵ group connects to. In some embodiments, W¹ is N. In some embodiments, W¹ is C. In some embodiments, s is 0. In some embodiments, s is 1. In some embodiments, R^{y}, at each occurrence, is independently and individually halogen, cyano, alkyl such as methyl, or haloalkyl such as CF₃. In some embodiments, R^{y}, at each occurrence, is independently and individually halogen, cyano, methyl, or CF₃. In some embodiments, W¹ is N, and s is 1. In some embodiments, W¹ is N, s is 1, and R^{y} is selected from halogen, methyl, CF₃, and cyano. In some embodiments, W¹ is N, and s is 0.

In some embodiments, at least two of W¹, W², W³, W⁴, and W⁵ are independently N or NH. For example, R^{H} is a diazole optionally substituted by R^{y}. In one implementation, R^{H} is an unsubstituted diazole. In one implementation, R^{H} is a diazole mono-substituted by halogen, methyl, CF₃, or cyano. In some embodiments, at least three of W¹, W², W³, W⁴, and W⁵ are independently N or NH. For example, R^{H} is a triazole optionally substituted by R^{y}. In one implementation, R^{H} is an unsubstituted triazole. In one implementation, R^{H} is a triazole mono-substituted by halogen, methyl, CF₃, or cyano. In some embodiments, at least four of W¹, W², W³, W⁴, and W⁵ are independently N or NH. For example, R^{H} is a tetrazole optionally substituted by R^{y}. In one implementation, R^{H} is an unsubstituted tetrazole. In one implementation, R^{H} is a tetrazole mono-substituted by halogen, methyl, CF₃, or cyano.

In some embodiments, R^{H} is selected from

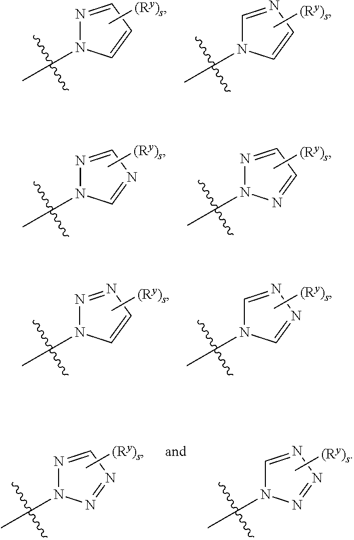

In some embodiments, R^{H} is

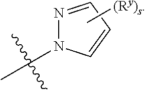

In some embodiments, R^{H} is

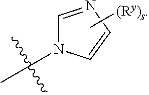

In some embodiments, R^{H} is

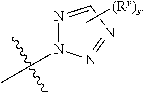

In some embodiments, R^{y}, at each occurrence, is independently and individually halogen, cyano, alkyl such as methyl, or haloalkyl such as CF₃. In some embodiments, R^{y}, at each occurrence, is independently and individually halogen, cyano, methyl, and CF₃.

In some embodiments, R^{H} is selected from

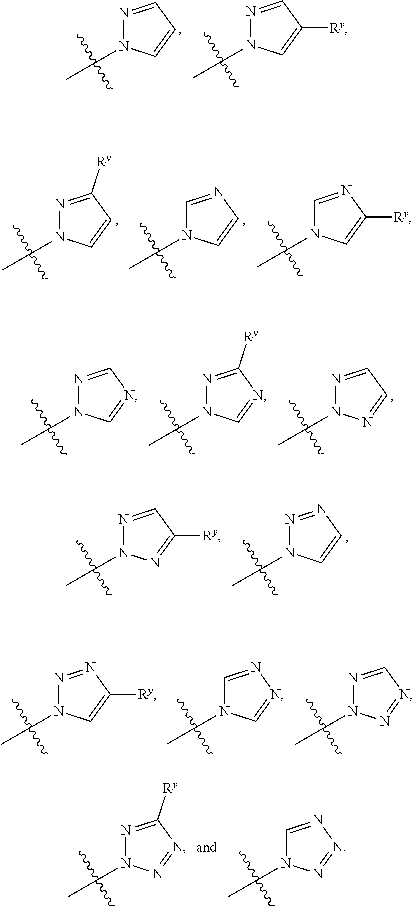

In some embodiments, R^{H} is

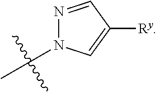

In some embodiments, R^{H} is

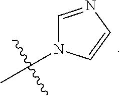 [SMILES: *n1ccnc1]

In some embodiments, R^{H} is

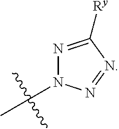

In some embodiments, R^{y}, if present, is halogen, cyano, alkyl such as methyl, or haloalkyl such as CF₃. In some embodiments, R^{y}, if present, is halogen, cyano, methyl, or CF₃.

In some embodiments, R^{H} is

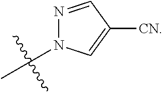 [SMILES: *n1cc(C#N)cn1]

In some embodiments, R^{H} is

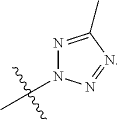 [SMILES: *n1nnc(C)n1]

In some embodiments, R^{H} is

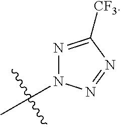 [SMILES: *n1nnc(C(F)(F)F)n1]

In some embodiments, R^{X} is hydrogen, i.e., Z¹ is —OH.

In some embodiments, R^{X} is acyl. For example, R^{X} is —C(═O)R^{e}, wherein R^{e} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle. In some embodiments, R^{e} is C₁-C₆ alkyl, halogen-substituted C₁-C₆ alkyl, C₃-C₆ cycloalkyl, or halogen-substituted C₃-C₆ cycloalkyl. In some embodiments, R^{e} is selected from methyl, —CF₃, ethyl, isopropyl, hexafluoroisopropyl, t-butyl, and cyclopropyl. In some embodiments, R^{e} is —CF₃. In some embodiments, R^{e} is isopropyl.

In some embodiments, R^{X} is ester. For example, R^{X} is —C(═O)OR^{c1}, wherein R^{c1} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. In some embodiments, R^{c1} is C₁-C₆ alkyl, halogen-substituted C₁-C₆ alkyl, C₃-C₆ cycloalkyl, or halogen-substituted C₃-C₆ cycloalkyl. In some embodiments, R^{c1} is selected from methyl, —CF₃, ethyl, isopropyl, hexafluoroisopropyl, t-butyl, and cyclopropyl. In some embodiments, R^{c1} is —CF₃. In some embodiments, R^{c1} is isopropyl.

In some embodiments, R^{X} is thioester. For example, R^{X} is —C(═O)SR^{o1}, wherein R^{o1} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. In some embodiments, R^{o1} is C₁-C₆ alkyl, halogen-substituted C₁-C₆ alkyl, C₃-C₆ cycloalkyl, or halogen-substituted C₃-C₆ cycloalkyl. In some embodiments, R^{o1} is selected from methyl, —CF₃, ethyl, isopropyl, hexafluoroisopropyl, t-butyl, and cyclopropyl. In some embodiments, R^{o1} is —CF₃. In some embodiments, R^{o1} is isopropyl.

In some embodiments, R^{X} is amide. For example, R^{X} is —C(═O)NR^{f1}R^{f2}, wherein R^{f1} and R^{f2} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same In some embodiments, R^{f1} and R^{f2} are hydrogen. In some embodiments, R^{f1} and R^{f2} are methyl. In some embodiments, R^{f1} is hydrogen and R^{f2} is methyl.

In some embodiments, Z² is methyl. In some embodiments, Z² is halogen-substituted methyl, such as —CH₂F, —CHF₂, or —CF₃.

In some embodiments, Z² is methoxymethyl. In some embodiments, Z² is halogen-substituted methoxymethyl, such as —CH₂OCF₃.

In some embodiments, Z² is selected from methyl, —CH₂F, —CHF₂, —CF₃, and —CH₂OCH₃. For example, Z² is selected from methyl, —CF₃, and —CH₂OCH₃.

In some embodiments, the neurosteroid moiety is selected from the following:

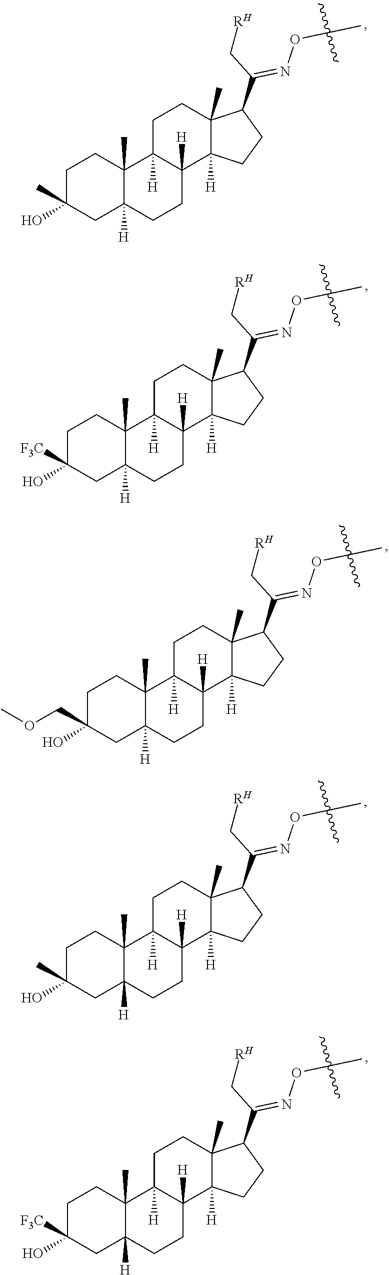

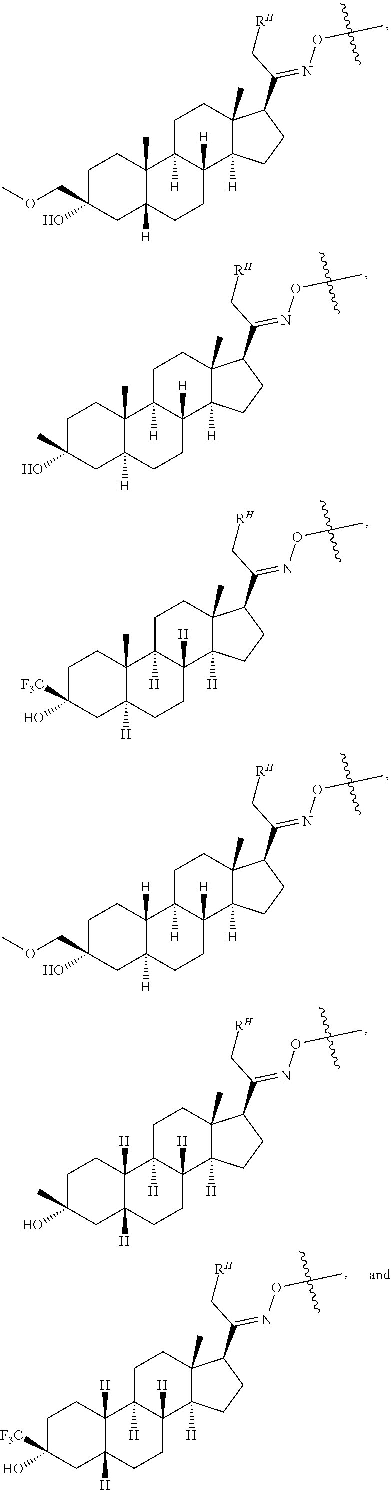

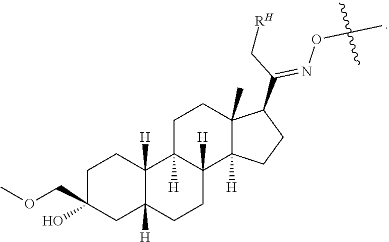

In some embodiments, R^{H} is H. In some embodiments, R^{H} is

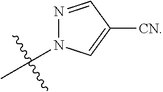 [SMILES: *n1cc(C#N)cn1]

In some embodiments, R^{H} is

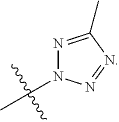 [SMILES: *n1nnc(C)n1]

In some embodiments, R^{H} is

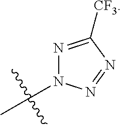 [SMILES: *n1nnc(C(F)(F)F)n1]

In some embodiments, R^{H} is

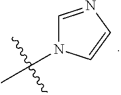 [SMILES: *n1ccnc1]

In some embodiments, the neurosteroid moiety is

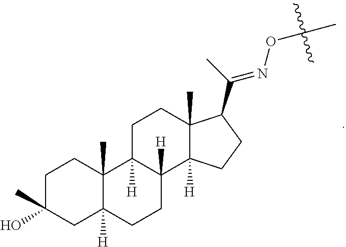

In some embodiments, the neurosteroid moiety is

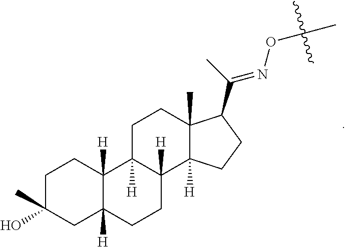

In some embodiments, the neurosteroid moiety is

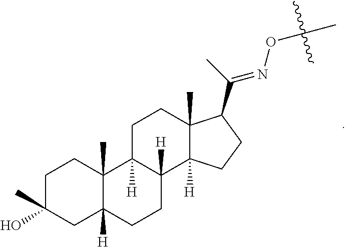

In some embodiments, the neurosteroid moiety is

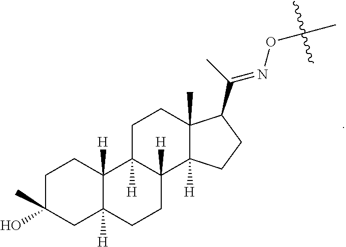

In some embodiments, the neurosteroid moiety is

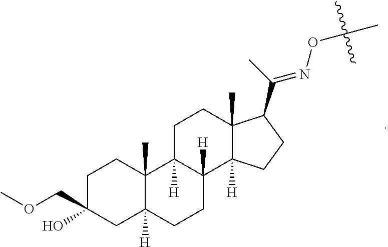

In some embodiments, the neurosteroid moiety is

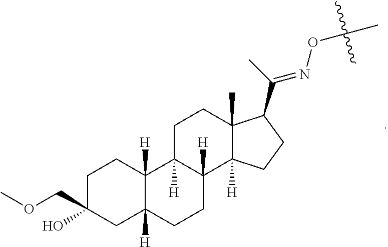

In some embodiments, the neurosteroid moiety is

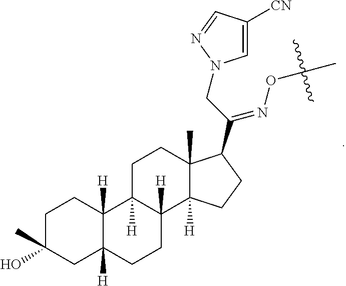

In some embodiments, the neurosteroid moiety is

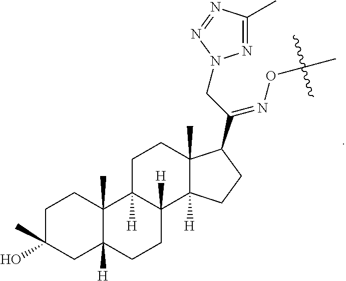

In some embodiments, the neurosteroid moiety is

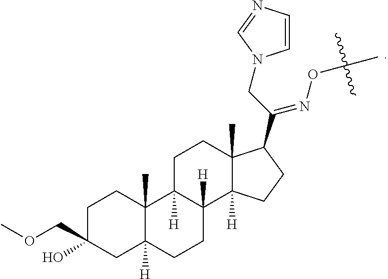

In some embodiments, the neurosteroid moiety is

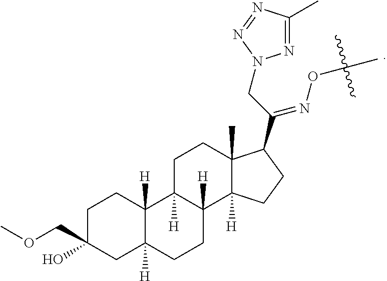

In some embodiments, the neurosteroid moiety is

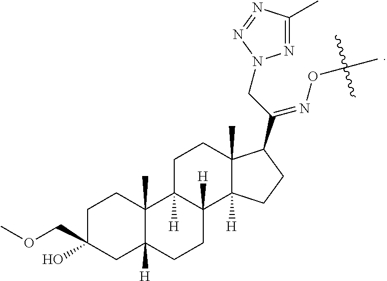

### 2. The Prodrug Moiety at the C20-Oxime

In some embodiments, T is O. In some embodiments, U is O. In some embodiments, V is O. In some embodiments, W is O. In some embodiments, T, U, V, and W are O.

In some embodiments, R⁶ is hydrogen or methyl. In some embodiments, R⁷ is hydrogen or methyl. In some embodiments, R⁶ is hydrogen and R⁷ is hydrogen. In some embodiments, R⁶ is hydrogen and R⁷ is methyl. In some embodiments, R⁶ is methyl and R⁷ is methyl.

In some embodiments, R⁸ is hydrogen, benzyl, or phenyl. In some embodiments, R⁹ is hydrogen, benzyl, or phenyl. In some embodiments, R¹ is hydrogen and R⁹ is hydrogen. In some embodiments, R⁸ is hydrogen and R⁹ is benzyl. In some embodiments, R⁸ is hydrogen and R⁹ is phenyl.

Optionally, Formula III and its sub-formulas may have the following features: T, U, V, and W are O; and R⁶, R⁷, R⁸, and R⁹ are hydrogen.

Optionally, Formula III and its sub-formulas may have the following features: T, U, V, and W are O; R⁶, R⁷, and R⁸ are hydrogen; and R⁹ is benzyl.

Optionally, Formula III and its sub-formulas may have the following features: T, U, V, and W are O; R⁶, R⁷, and R⁸ are hydrogen; and R⁹ is phenyl.

Optionally, Formula III and its sub-formulas may have the following features: T, U, V, and W are O; R⁶ and R⁷ are methyl; and R⁸ and R⁹ are hydrogen.

Optionally, Formula III and its sub-formulas may have the following features: T, U, V, and W are O; R⁶ and R⁷ are methyl; R⁸ is hydrogen; and R⁹ is benzyl.

Optionally, Formula III and its sub-formulas may have the following features: T, U, V, and W are O; R⁶ and R⁷ are methyl; R⁸ is hydrogen; and R⁹ is phenyl.

### 3. Exemplary Structures

In some embodiments, the prodrugs have a structure of Formula III or a pharmaceutically acceptable salt, hydrate, or hydrated salt thereof,

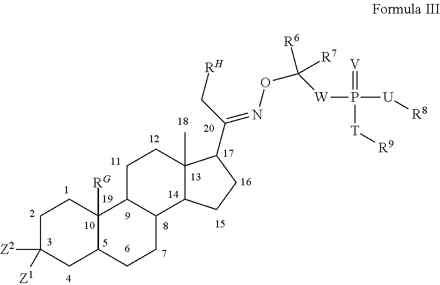

wherein the

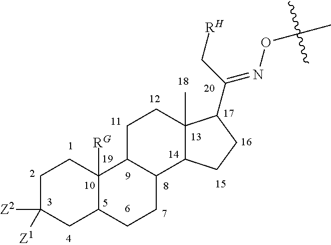

moiety is selected from:

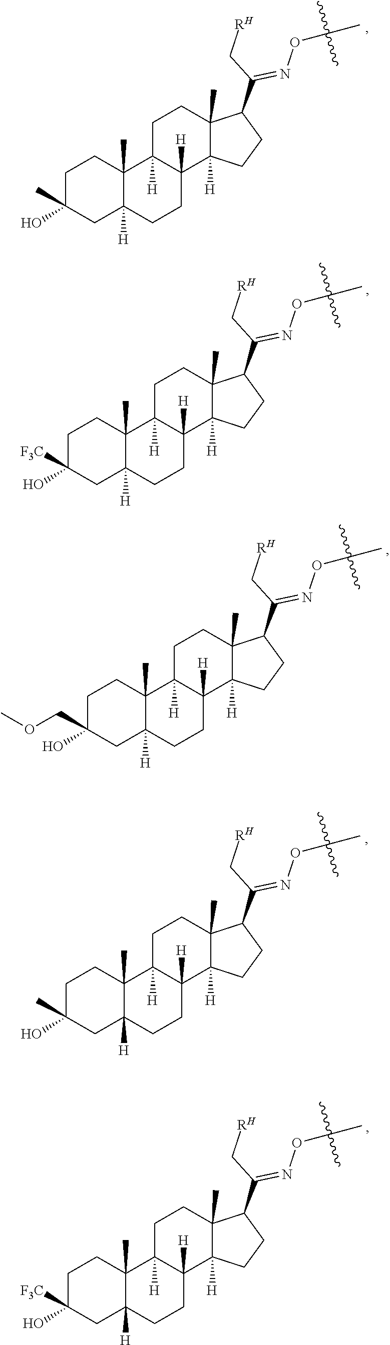

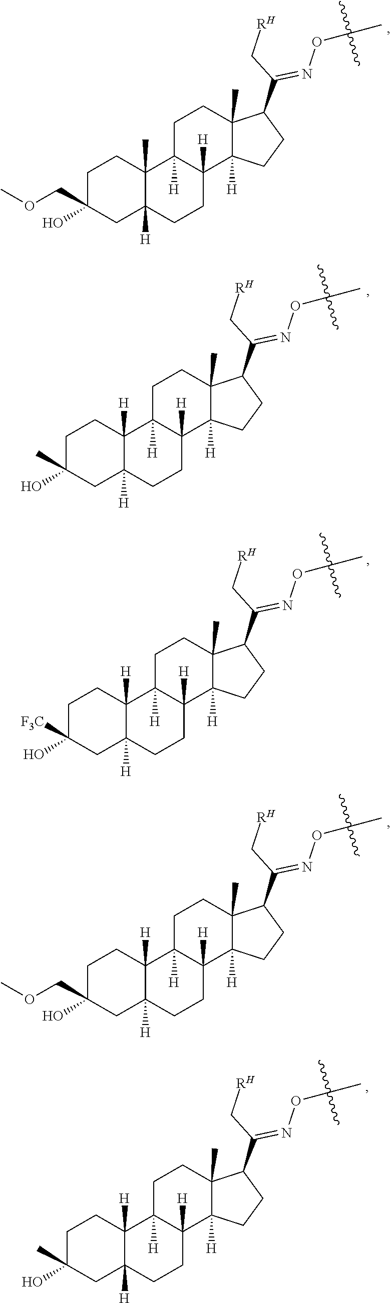

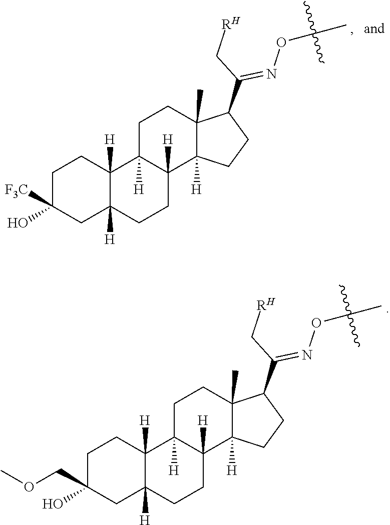

wherein R^{H} is selected from H, and

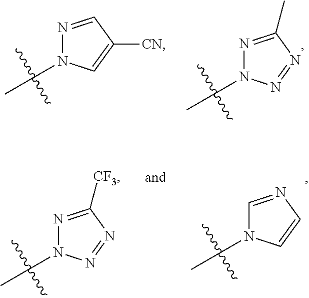

wherein T, U, V, and W are O,
wherein R⁶ is hydrogen or methyl,
wherein R⁷ is hydrogen or methyl,
wherein R⁸ is hydrogen, benzyl, or phenyl, and
wherein R⁹ is hydrogen, benzyl, or phenyl.

In some embodiments, the

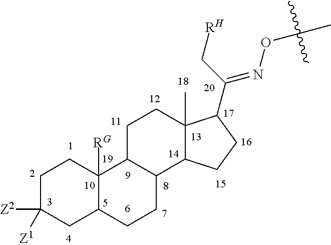

moiety is

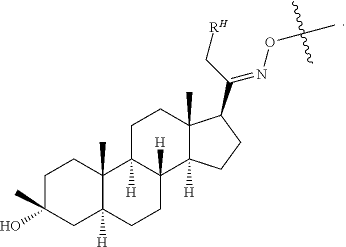

In some embodiments, the

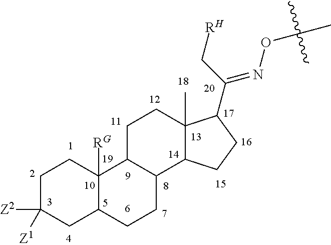

moiety is

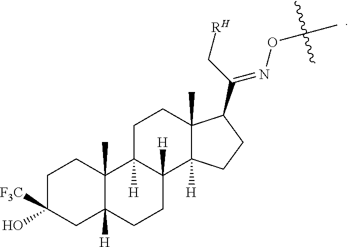

In some embodiments, the

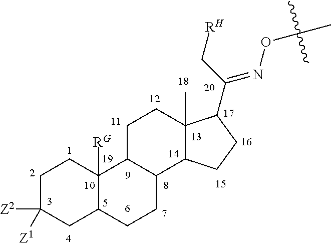

moiety is

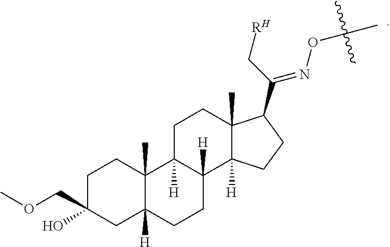

In some embodiments, the

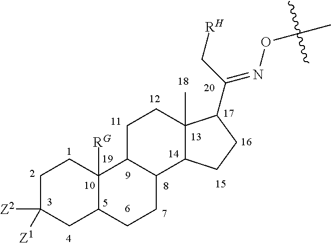

moiety is

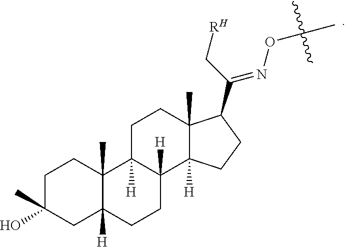

In some embodiments, the

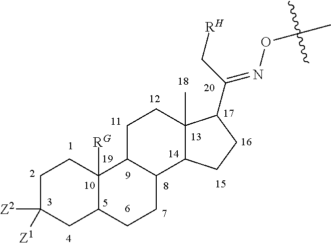

moiety is

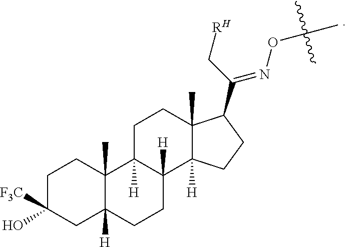

In some embodiments, the

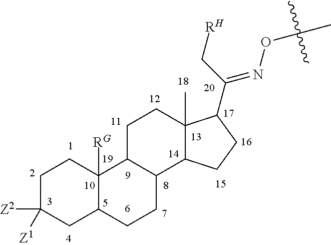

moiety is

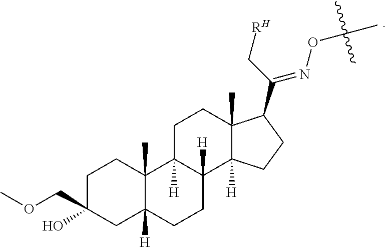

In some embodiments, the

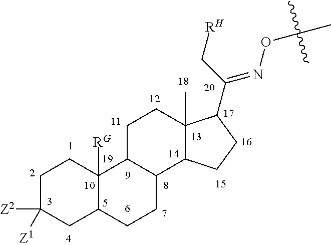

moiety is

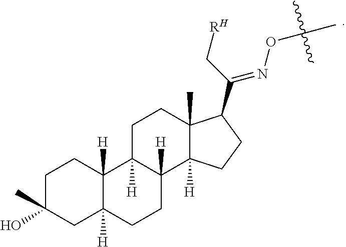

In some embodiments, the

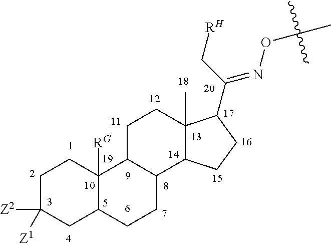

moiety is

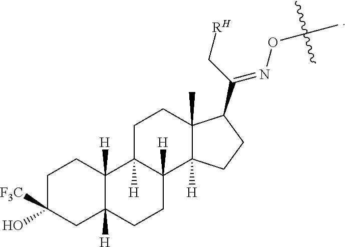

In some embodiments, the

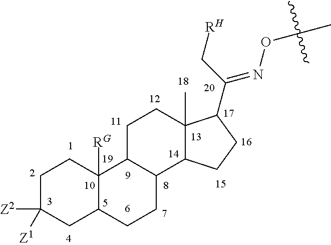

moiety is

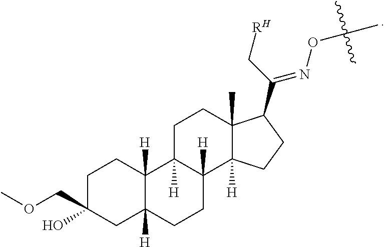

In some embodiments, the

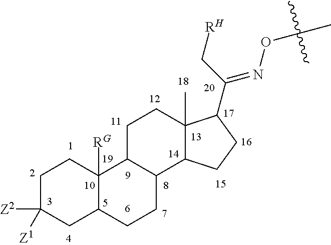

moiety is

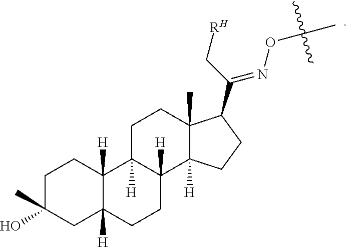

In some embodiments, the

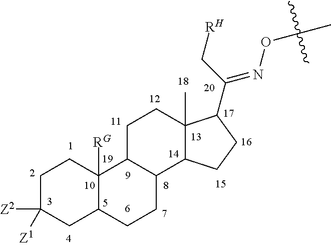

moiety is

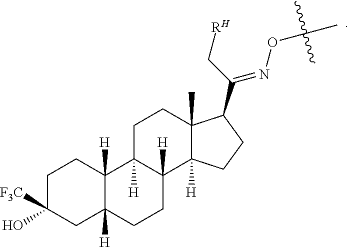

In some embodiments, the

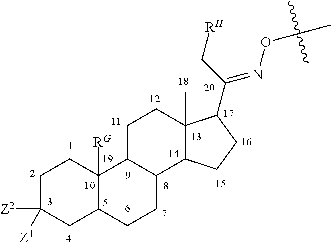

moiety is

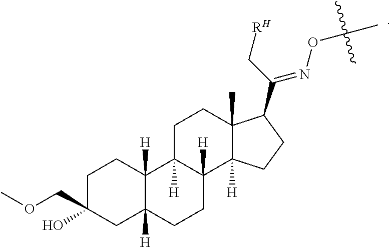

In some embodiments, R^{H} is H. In some embodiments R^{H} is

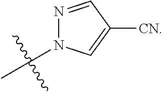 [SMILES: *n1cc(C#N)cn1]

In some embodiments, R^{H} is

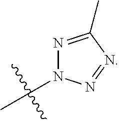 [SMILES: *n1nnc(C)n1]

In some embodiments, R^{H} is

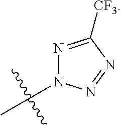 [SMILES: *n1nnc(C(F)(F)F)n1]

In some embodiments, R^{H} is

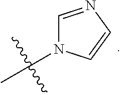 [SMILES: *n1ccnc1]

In some embodiments, R⁶, R⁷, R⁸, and R⁹ are hydrogen. In some embodiments, R⁶, R⁷, and R⁸ are hydrogen, and R⁹ is benzyl. In some embodiments, R⁶, R⁷, and R⁸ are hydrogen, and R⁹ is phenyl. In some embodiments, R⁶ and R⁷ are methyl, and R⁸ and R⁹ are hydrogen. In some embodiments, R⁶ and R⁷ are methyl, R⁸ is hydrogen, and R⁹ is benzyl. In some embodiments, R⁶ and R⁷ are methyl, R⁸ is hydrogen, and R⁹ is phenyl.

In some embodiments, the foregoing exemplified prodrugs are in a non-salt form as shown in the structures. In some embodiments, the prodrugs are in a salt form. In some embodiments, the prodrugs are in a Tris or bis-Tris salt form.

### D. Deuterated or Halogenated Analogs

### 1. Deuterated Analogs

This disclosure also provides deuterated analogs of (1) the neurosteroids described above in Sections II(A) of the Detailed Description and (2) the prodrugs of the neurosteroids described above in Sections II(B) and II(C) of the Detailed Description, wherein one or more non-ionizable hydrogen atoms in the

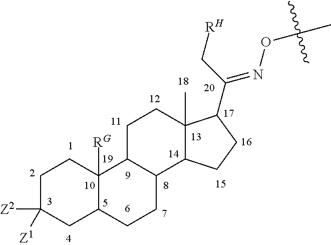

moiety of (1) the neurosteroids and (2) the prodrugs of the neurosteroids are replaced with deuterium.

Consistent with their corresponding non-deuterated versions, the deuterated analogs may have a structure of Formula I and its sub-formulas (Formula I-1, Formula I-2), Formula II and its sub-formulas (Formula II-1, Formula II-2, Formula IIA, Formula IIA-1, Formula IIA-2, Formula IIB, Formula IIB-1, Formula IIB-2, Formula IIC, Formula IIC-1, Formula IIC-2, Formula IID, Formula IID-1, Formula IID-2), or Formula III and its sub-formulas (Formula III-1, Formula III-2), as described above, or a pharmaceutically acceptable salt, hydrate, or hydrated salt thereof.

In some embodiments, the deuterated

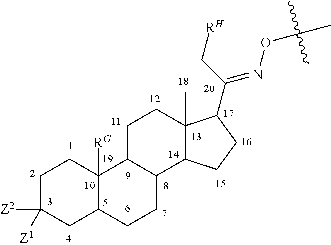

moiety is fully deuterated, i.e., all the non-ionizable hydrogen atoms in this moiety are replaced with deuterium.
In some embodiments, the deuterated

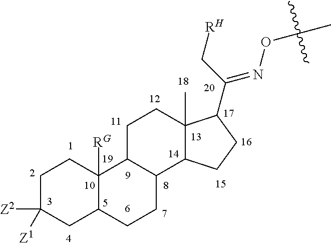

moiety is partially deuterated, i.e., one or more non-ionizable hydrogen atoms, but not all the non-ionizable hydrogen atoms, in this moiety are replaced with deuterium.

In some embodiments, the deuterated analogs have enhanced metabolic stability. The enhanced metabolic stability may origin from the kinetic isotope effect (KIE), e.g., an elevated energy barrier associated with cytochrome P450-mediated deuterium abstraction, compared to hydrogen abstraction. Notably, the neurosteroids disclosed herein can be oxidatively modified at the C17 position (e.g., hydroxylation) and/or at the C4/C5/C6 position (e.g., formation of C4═C5 alkene or C5═C6 alkene) by cytochrome P450s. Accordingly, installation of deuterium at one or more of these positions in the neurosteroids and the prodrugs of the neurosteroids can deter the oxidative modification events, thereby enhancing metabolic stability.

In some embodiments, the enhanced metabolic stability corresponds to a larger HLM t_{1/2}, RLM t_{1/2}, or MLM t_{1/2}. An exemplary method of measuring HLM t_{1/2}, RLM t_{1/2}, and MLM t_{1/2} is described in Example 8. In the context of the prodrugs, the HLM t_{1/2}, RLM t_{1/2}, and MLM t_{1/2} values refer to those determined using the corresponding neurosteroid parent compounds.

In some embodiments, the enhanced metabolic stability corresponds to a higher oral bioavailability in an animal model (such as mouse, rat, dog, or non-human primate) or human.

Methods of making exemplary deuterated analogs are disclosed in subsequent sections and exemplified by the Examples. The synthetic methods disclosed herein are compatible with a wide variety of functional groups and starting materials. Thus, a wide variety of deuterated analogs can be obtained from the disclosed methods.

In some embodiments, the hydrogen atom at the C17 position is replaced with deuterium.

In some embodiments, the hydrogen atom at the C5 position is replaced with deuterium.

In some embodiments, the two hydrogen atoms at the C4 position are replaced with deuterium.

In some embodiments, the two hydrogen atoms at the C6 position are replaced with deuterium.

In some embodiments, the two hydrogen atoms of the methylene group at the C20 position are replaced with deuterium. In some embodiments, when R^{H} is hydrogen, the corresponding methyl group at the C20 position is fully deuterated.

In some embodiments, Z² is deuterated, either fully deuterated or partially deuterated. In some embodiments, Z² is a fully deuterated methyl (—CD₃). In some embodiments, Z² is deuterated —CH₂OCH₃, such as —CD₂OCH₃, —CH₂OCD₃, or —CD₂ OCD₃.

In some embodiments, the hydrogen atom at the C17 position is replaced with deuterium, and the hydrogen atom at the C5 position is replaced with deuterium.

In some embodiments, the hydrogen atom at the C17 position is replaced with deuterium, and the two hydrogen atoms of the methylene group at the C20 position are replaced with deuterium. In some embodiments, the hydrogen atom at the C17 position is replaced with deuterium, and when R^{H} is hydrogen, the corresponding methyl group at the C20 position is fully deuterated.

In some embodiments, the hydrogen atom at the C5 position is replaced with deuterium, and Z² is deuterated, either fully deuterated or partially deuterated. In some embodiments, Z² is a fully deuterated methyl (—CD₃). In some embodiments, Z² is deuterated —CH₂OCH₃, such as —CD₂OCH₃, —CH₂OCD₃, or —CD₂OCD₃.

In some embodiments, the hydrogen atom at the C17 position is replaced with deuterium, the hydrogen atom at the C5 position is replaced with deuterium, and the methyl group at the C20 position is fully deuterated.

In some embodiments, the hydrogen atom at the C17 position is replaced with deuterium, the hydrogen atom at the C5 position is replaced with deuterium, and Z² is deuterated, either fully deuterated or partially deuterated. In some embodiments, Z² is a fully deuterated methyl (—CD₃).

In some embodiments, Z² is deuterated —CH₂OCH₃, such as —CD₂OCH₃, —CH₂OCD₃, or —CD₂OCD₃.

In some embodiments, the hydrogen atom at the C17 position is replaced with deuterium, the hydrogen atom at the C5 position is replaced with deuterium, the two hydrogen atoms of the methylene group at the C20 position are replaced with deuterium group, and Z² is fully deuterated or partially deuterated. In some embodiments, the hydrogen atom at the C17 position is replaced with deuterium; the hydrogen atom at the C5 position is replaced with deuterium; when R^{H} is hydrogen, the corresponding methyl group at the C20 position is fully deuterated; and Z² is fully deuterated or partially deuterated. In some embodiments, Z² is a fully deuterated methyl (—CD₃).

In some embodiments, Z² is deuterated —CH₂OCH₃, such as —CD₂OCH₃, —CH₂OCD₃, or —CD₂OCD₃.

In some embodiments, the deuterated

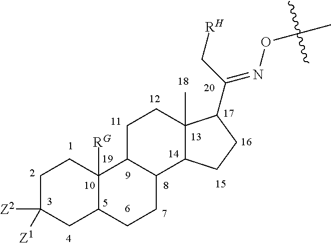

moiety is selected from:

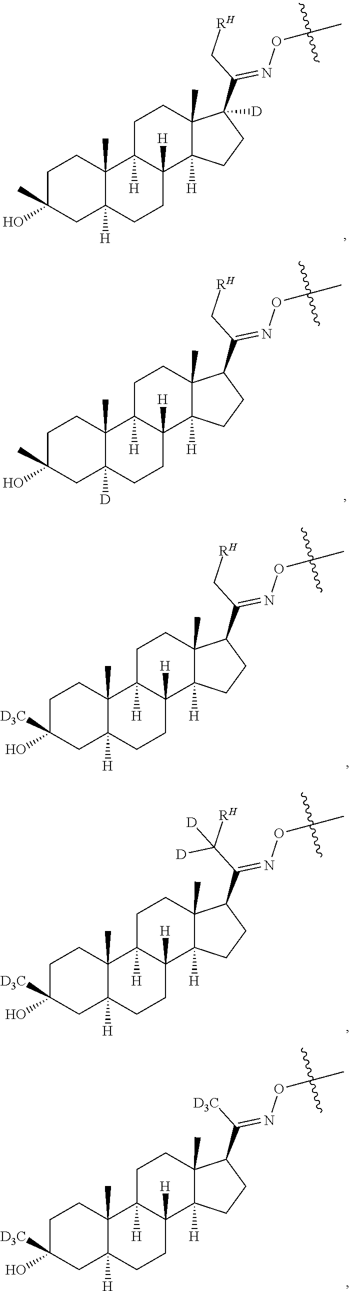

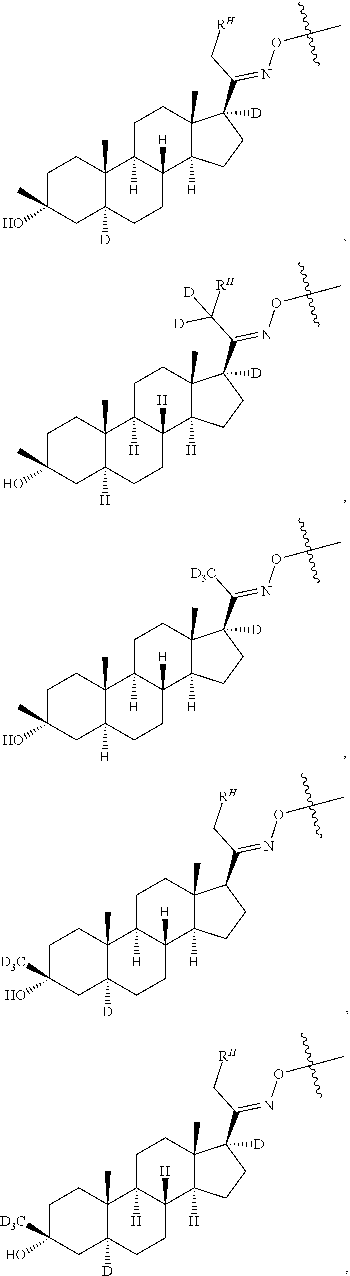

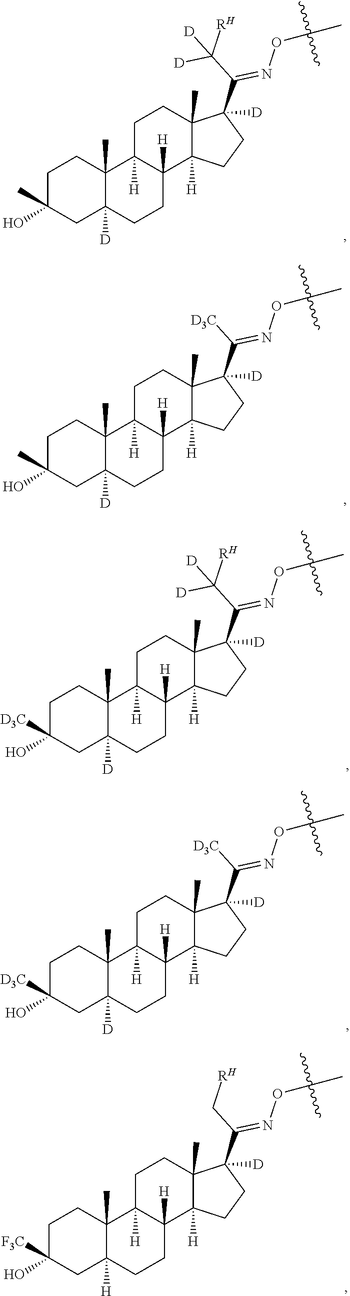

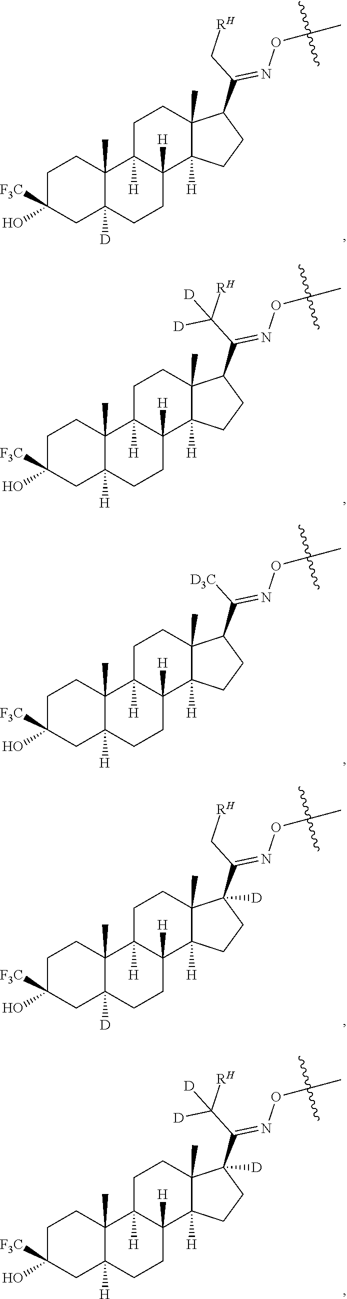

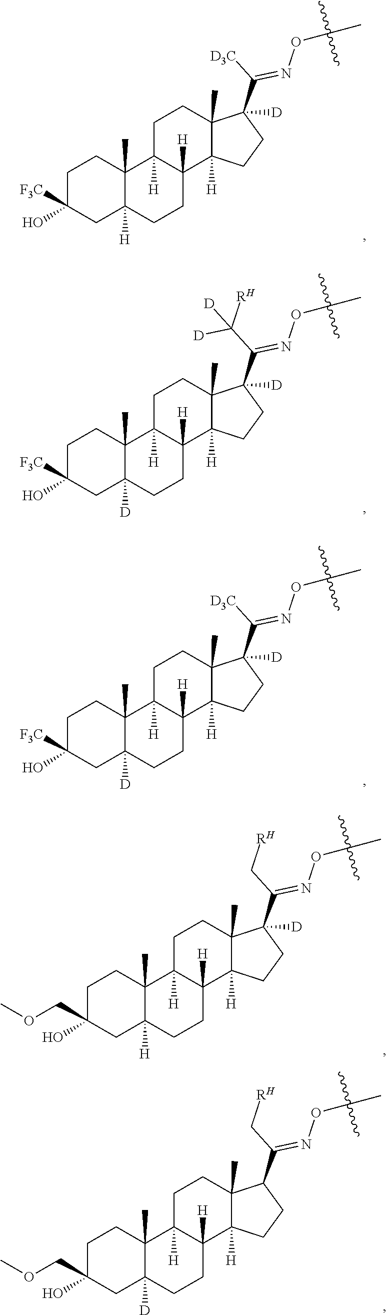

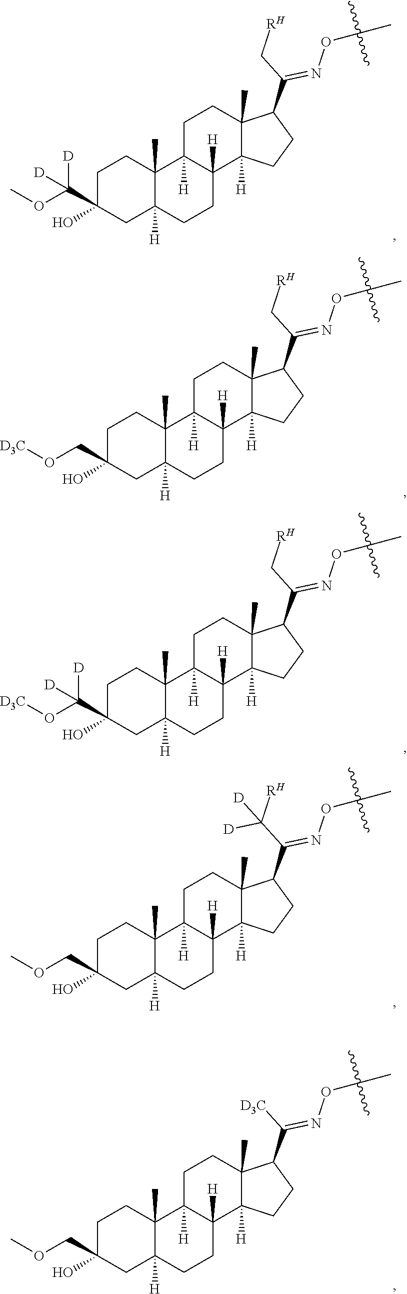

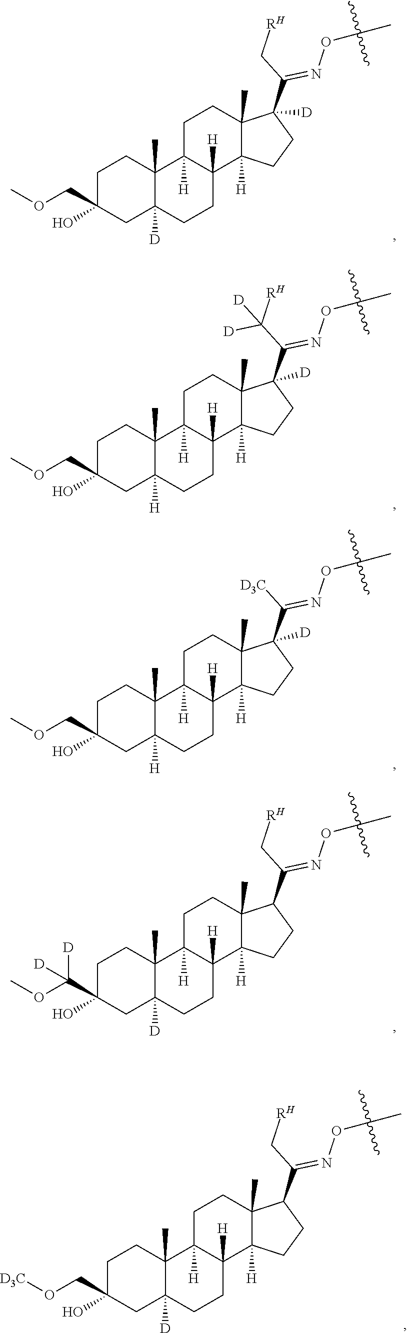

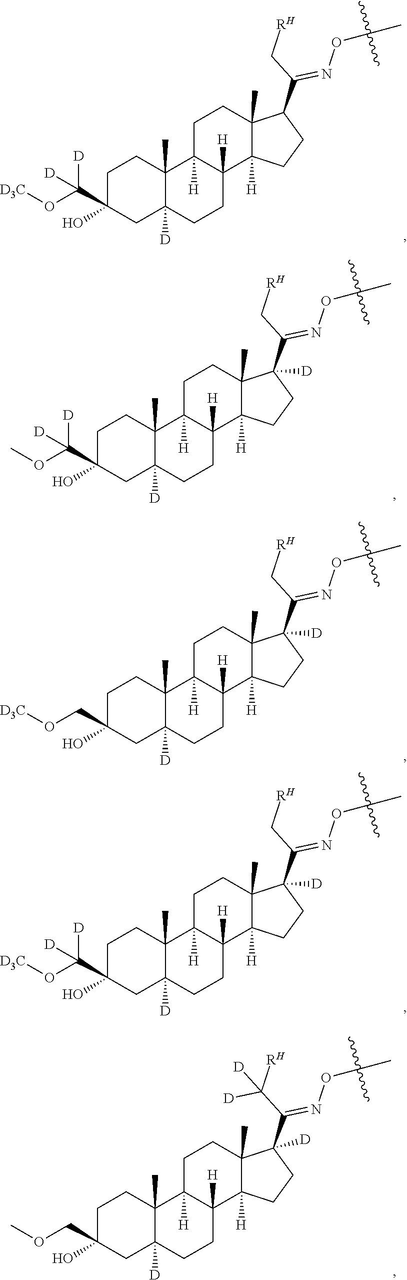

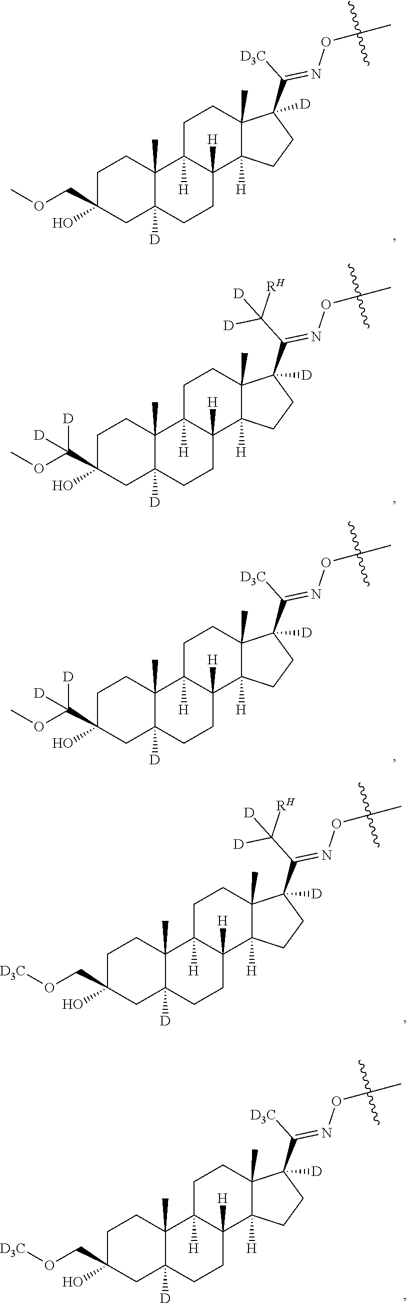

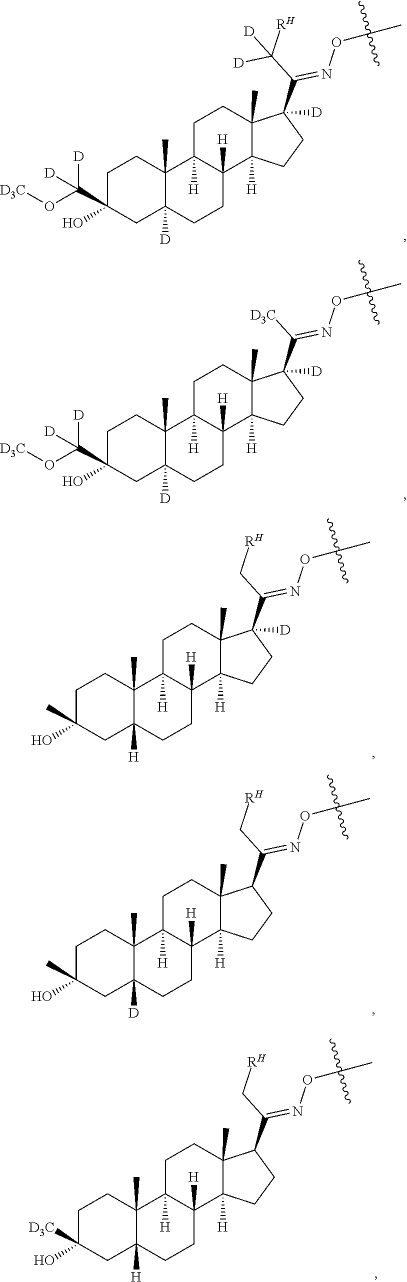

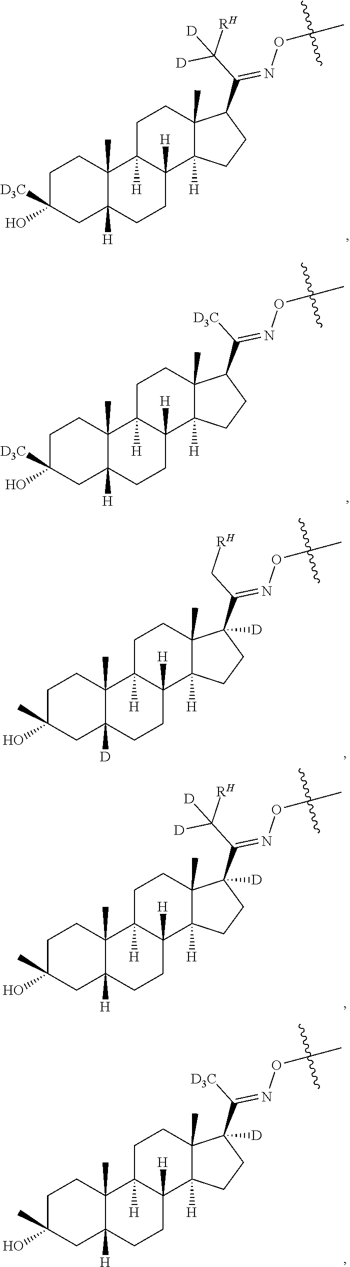

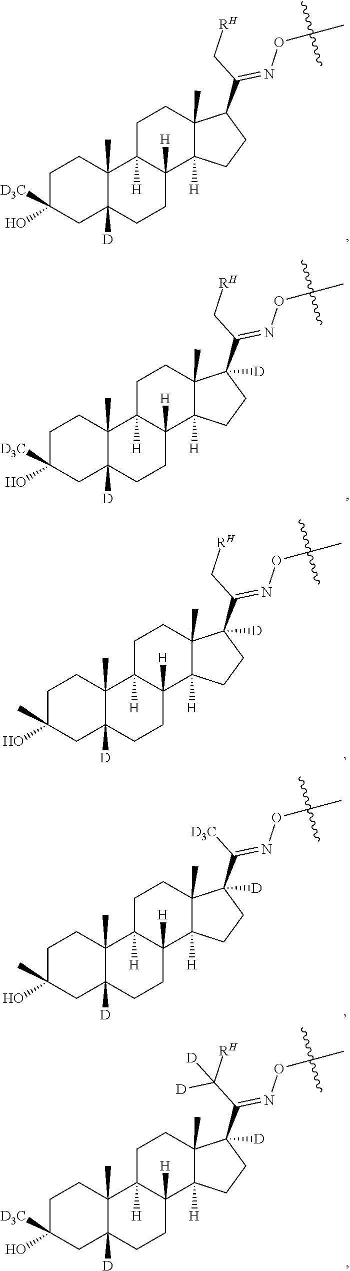

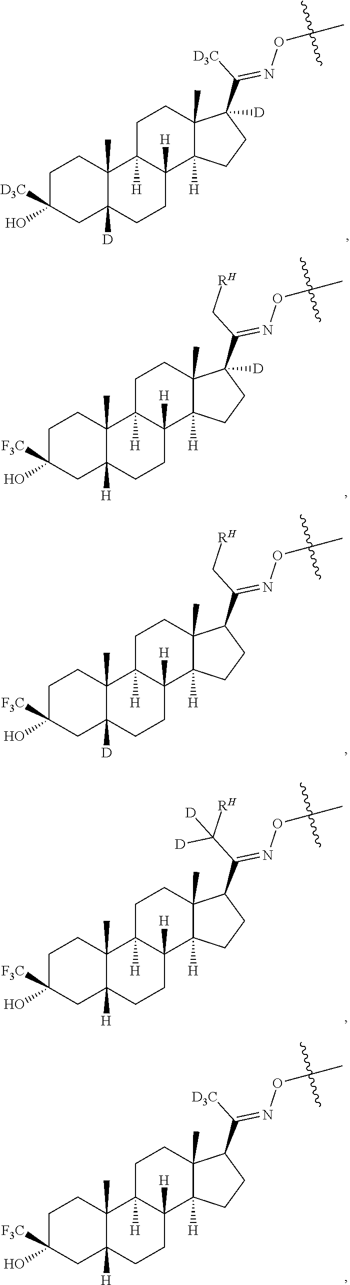

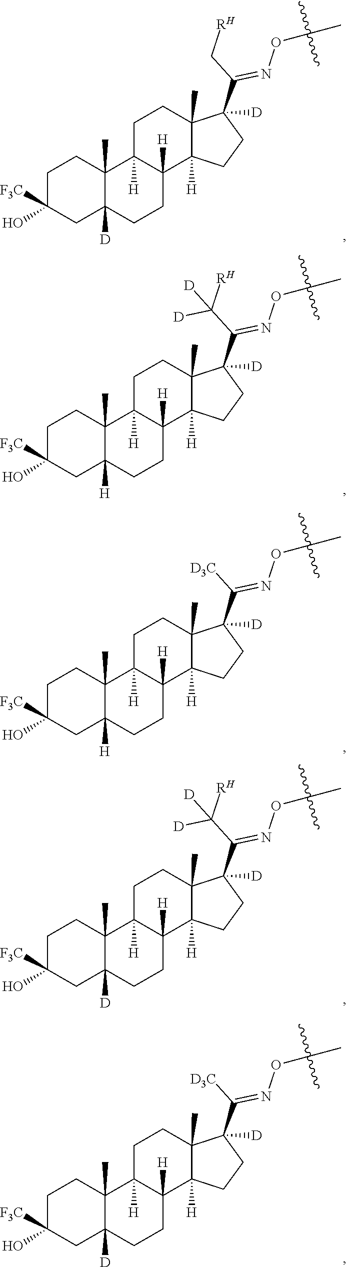

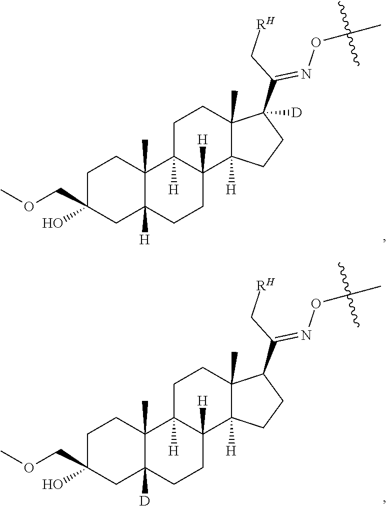

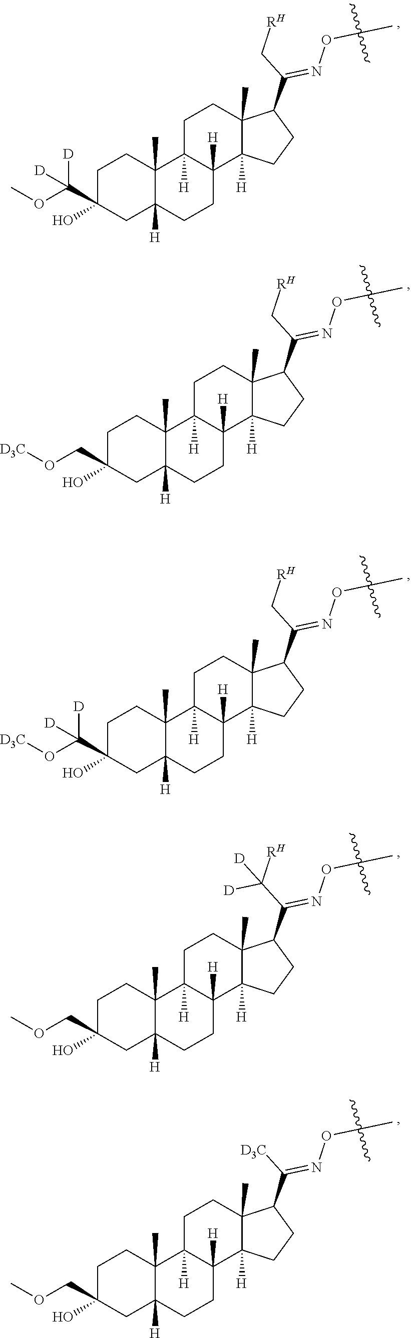

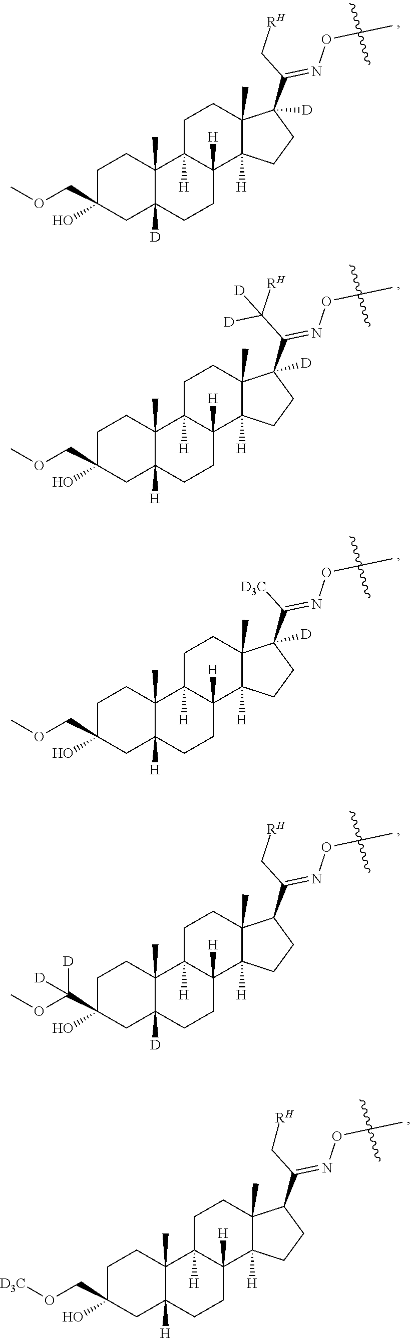

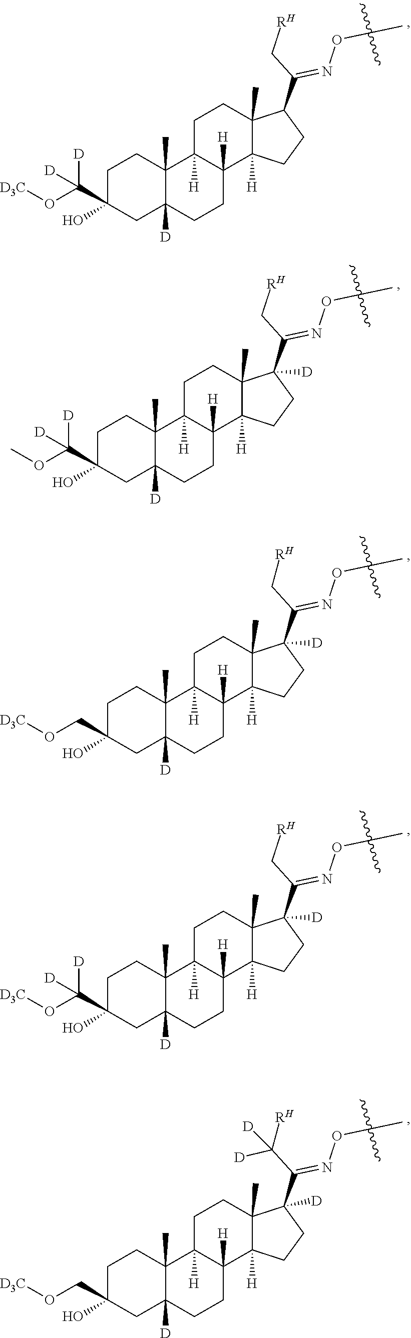

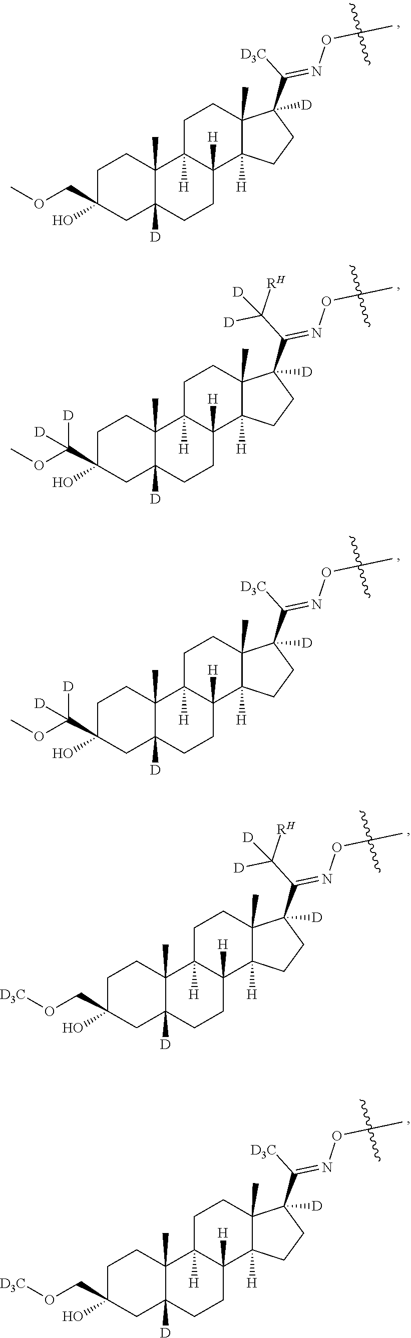

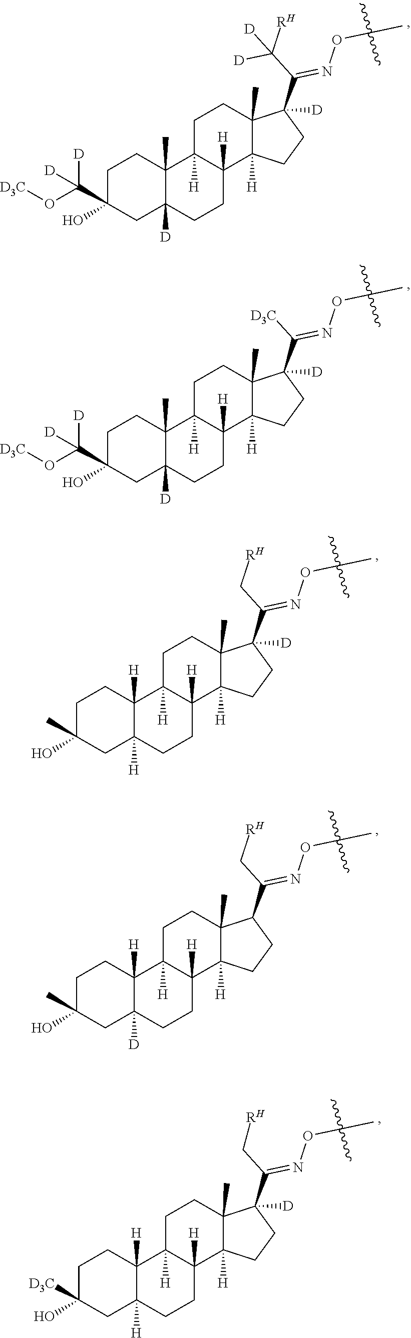

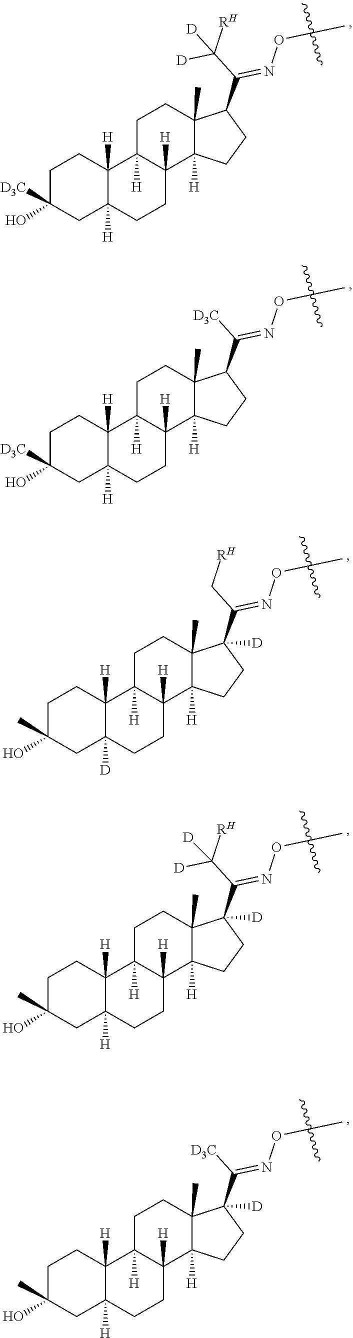

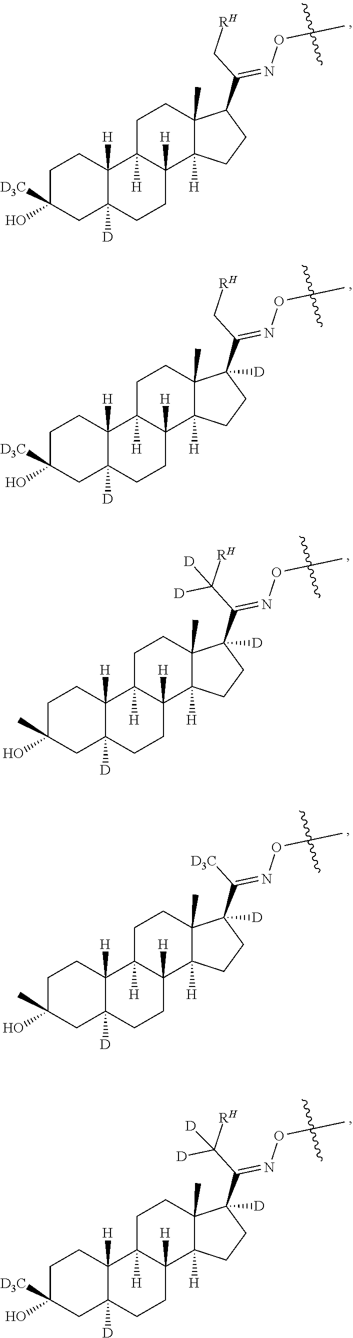

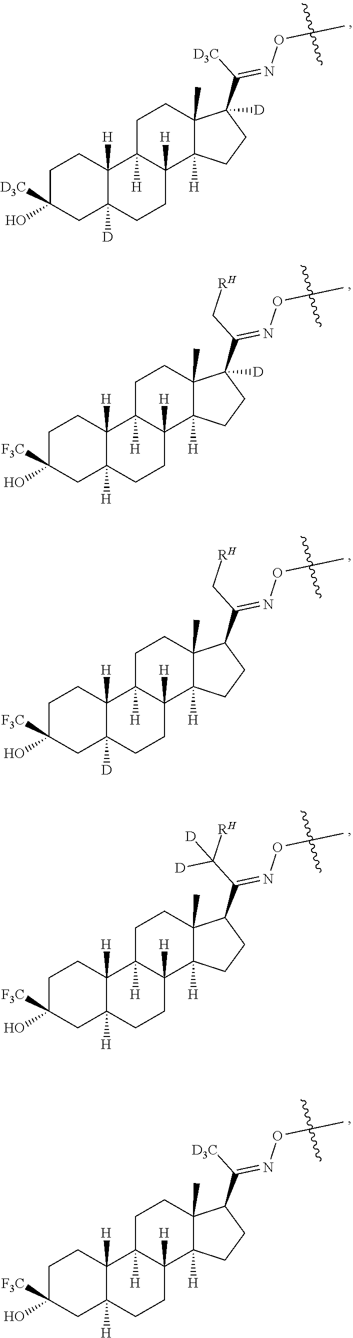

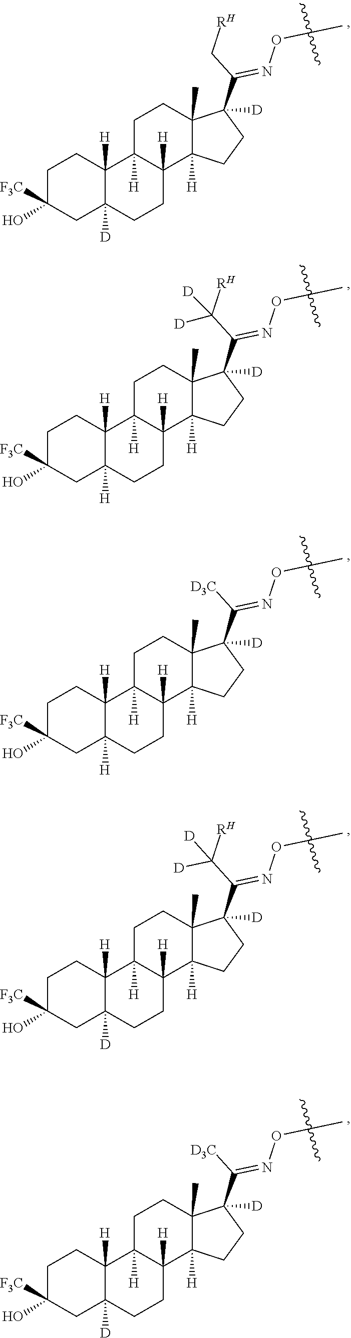

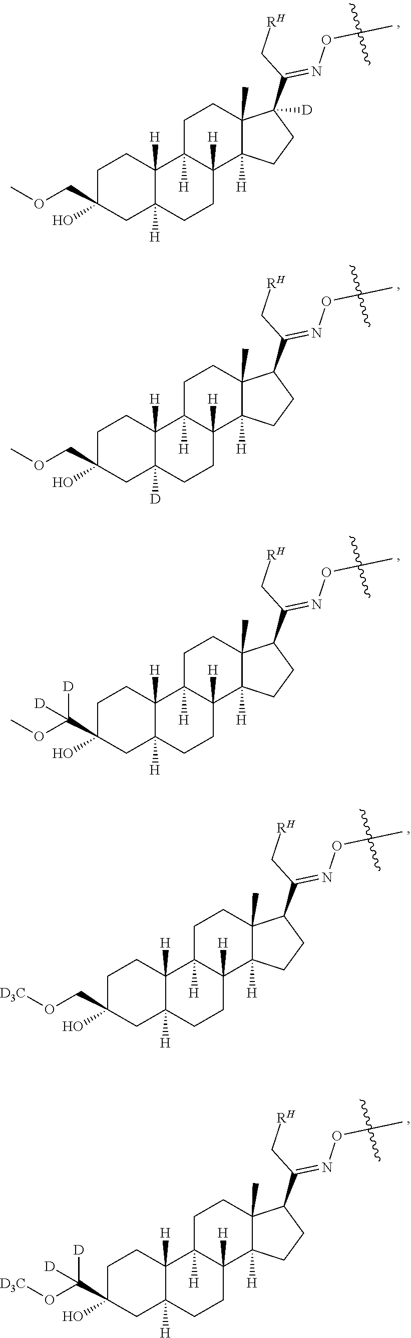

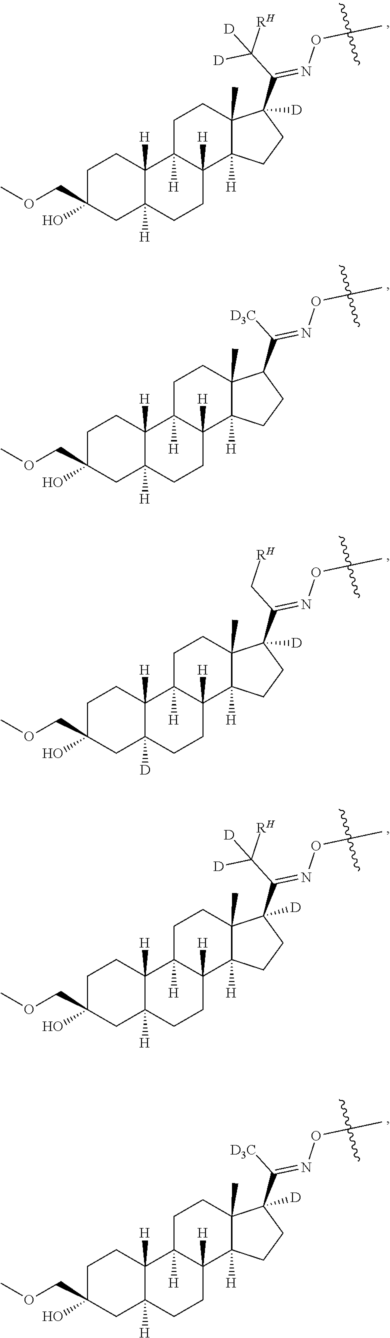

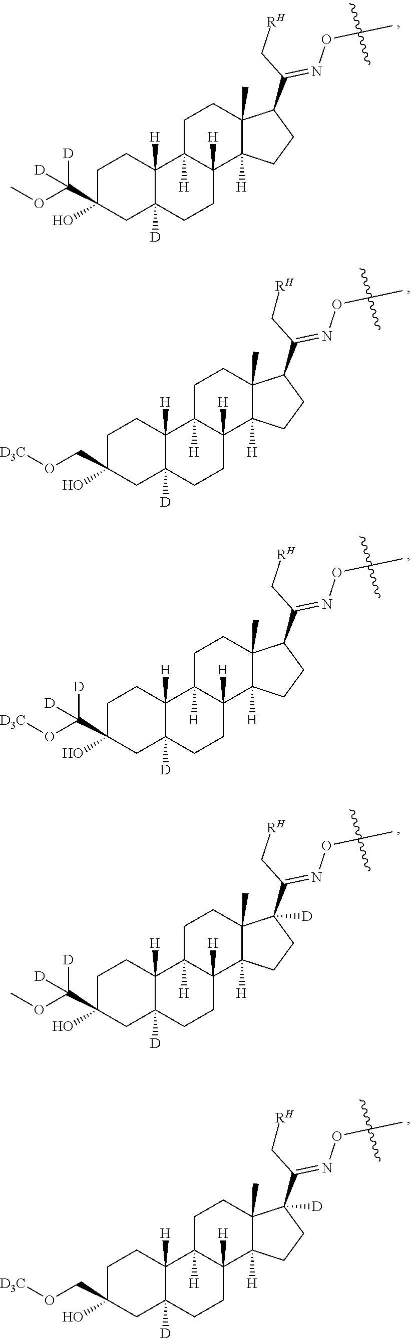

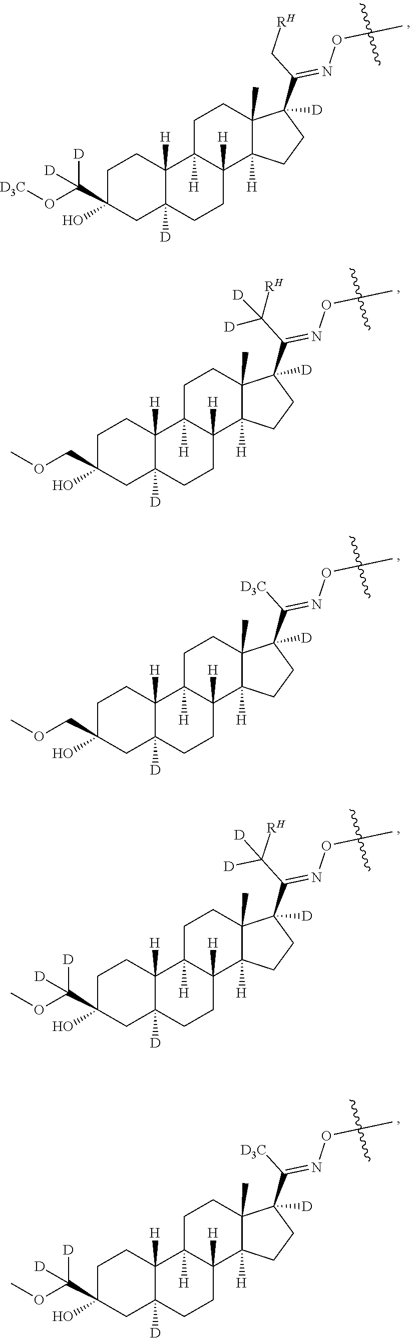

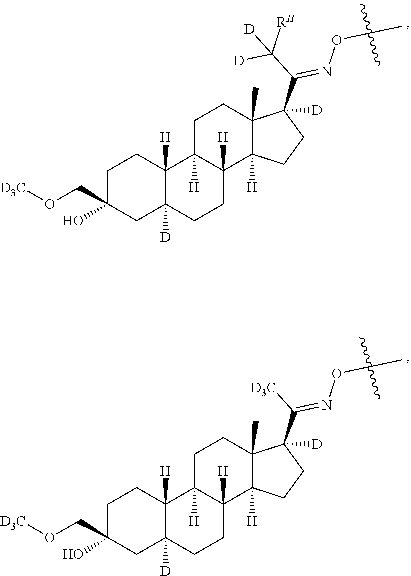

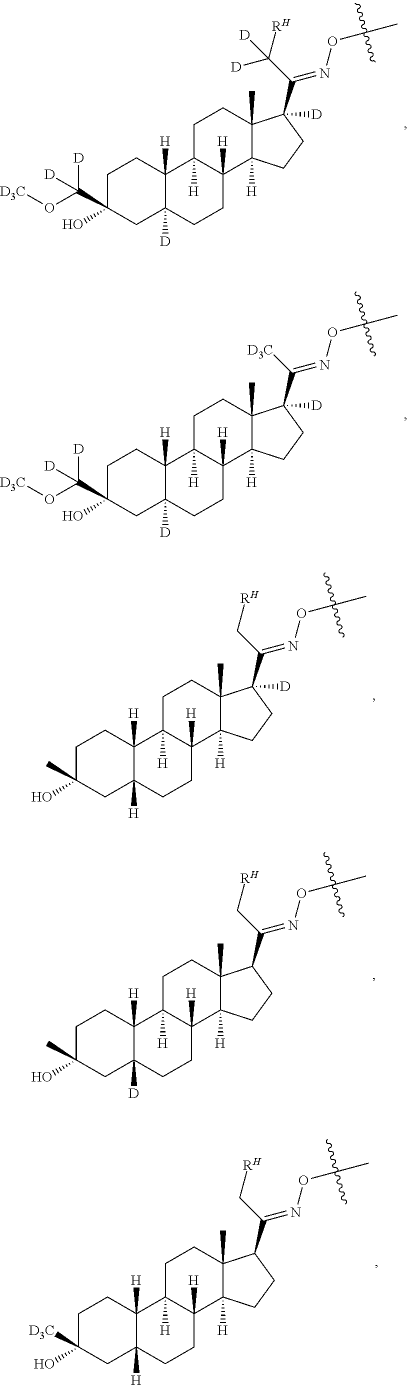

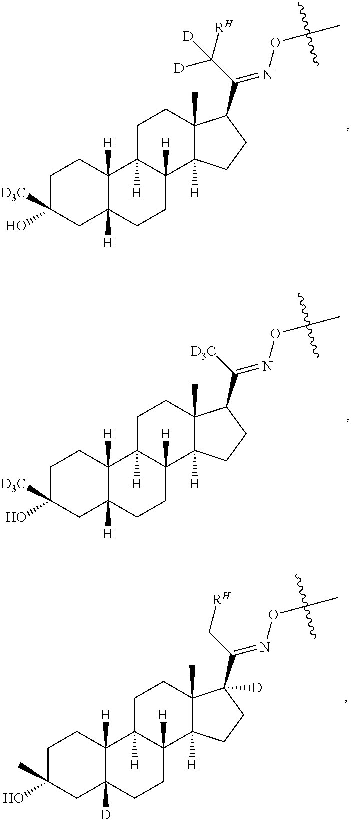

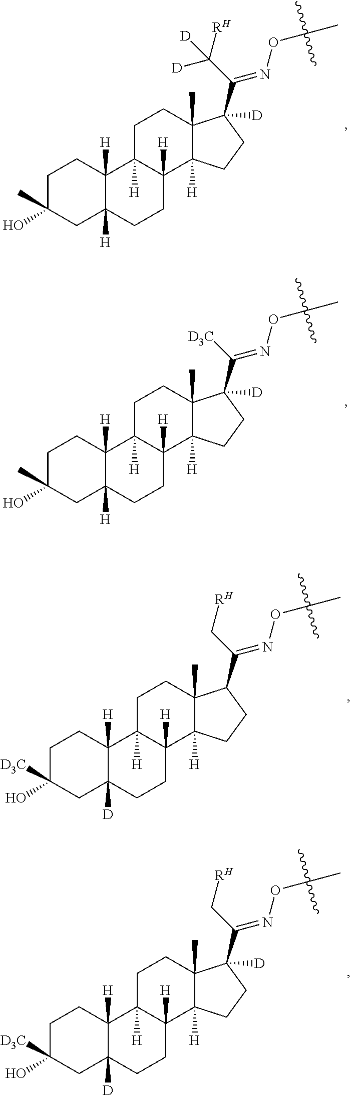

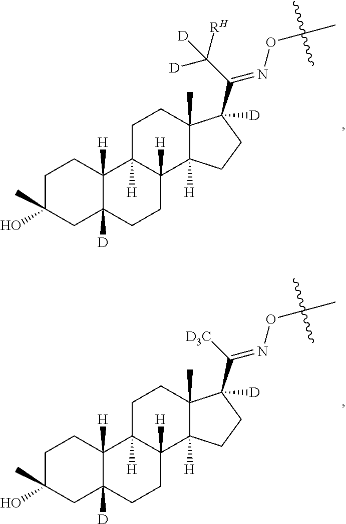

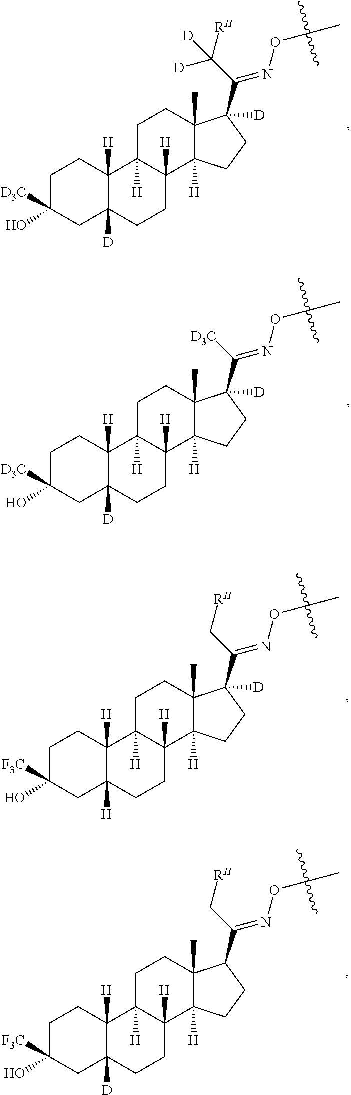

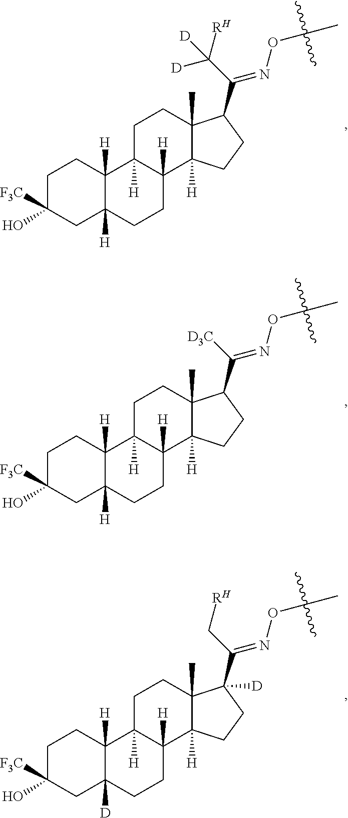

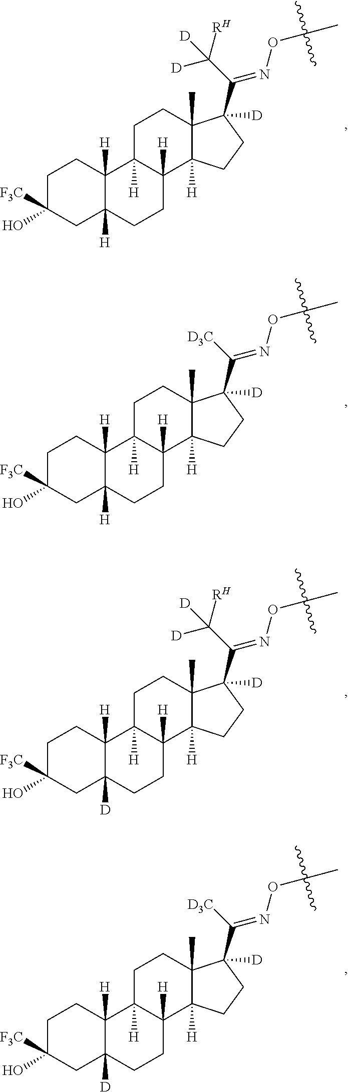

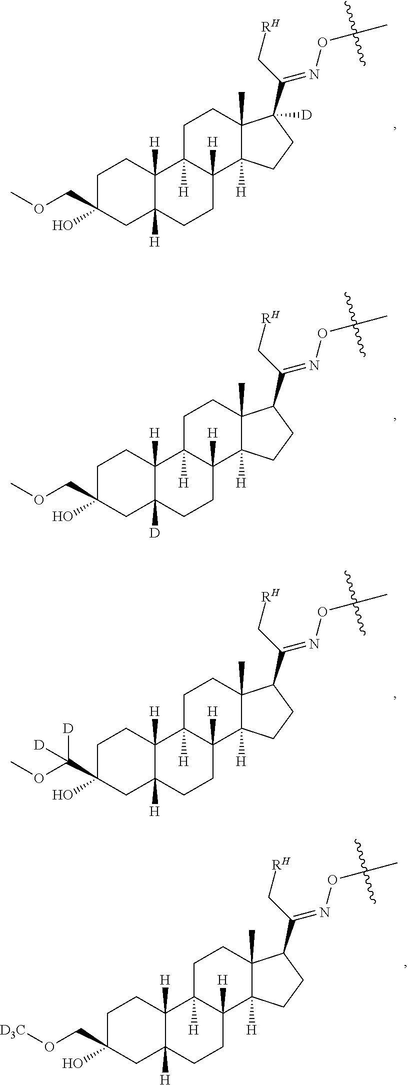

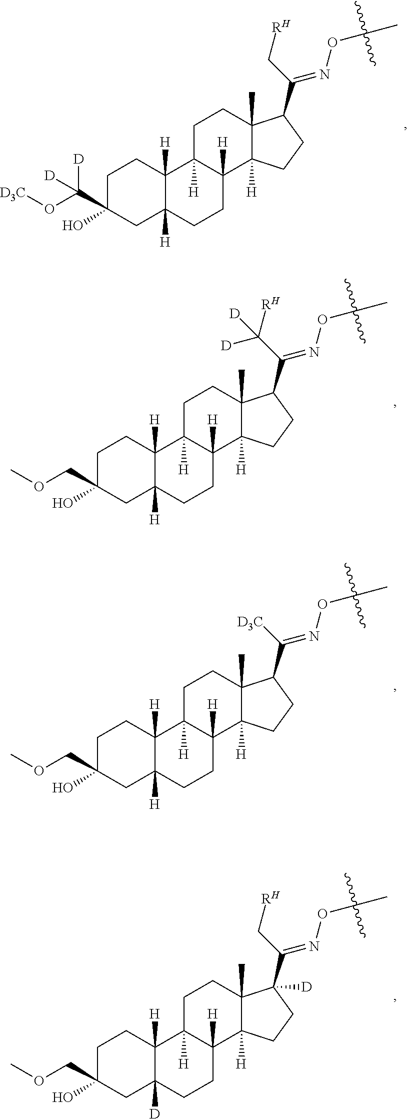

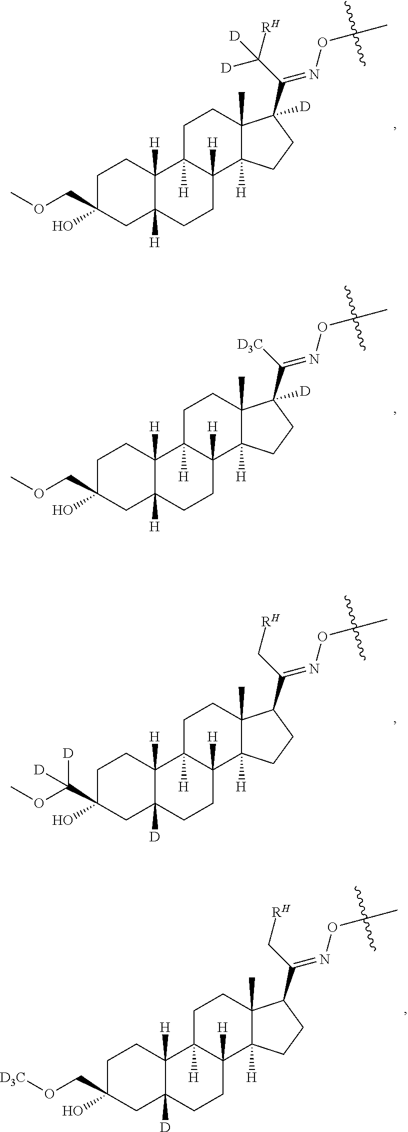

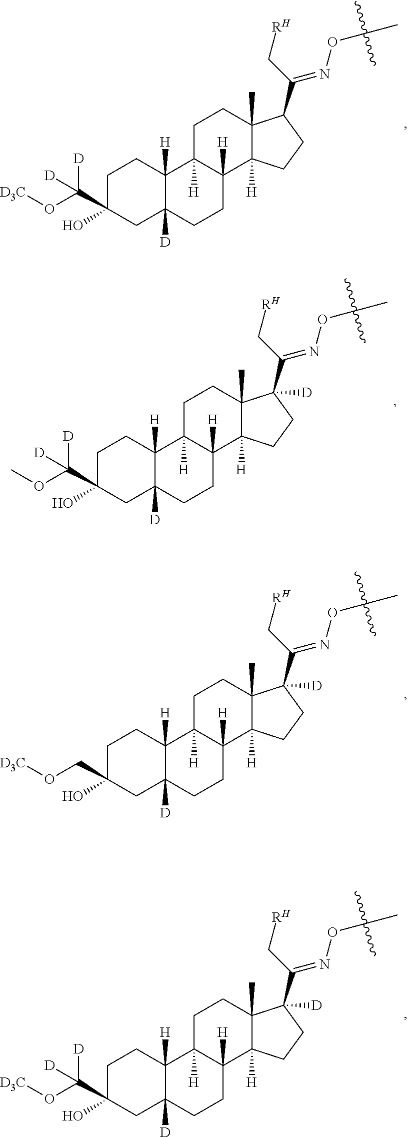

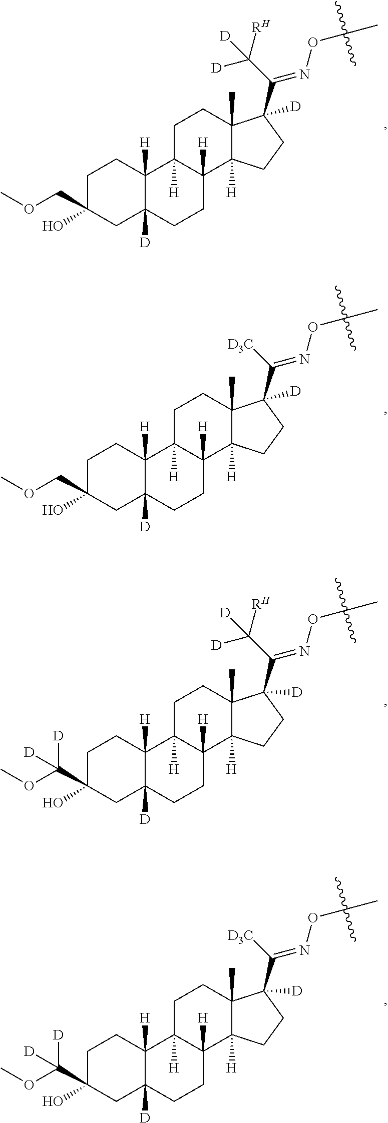

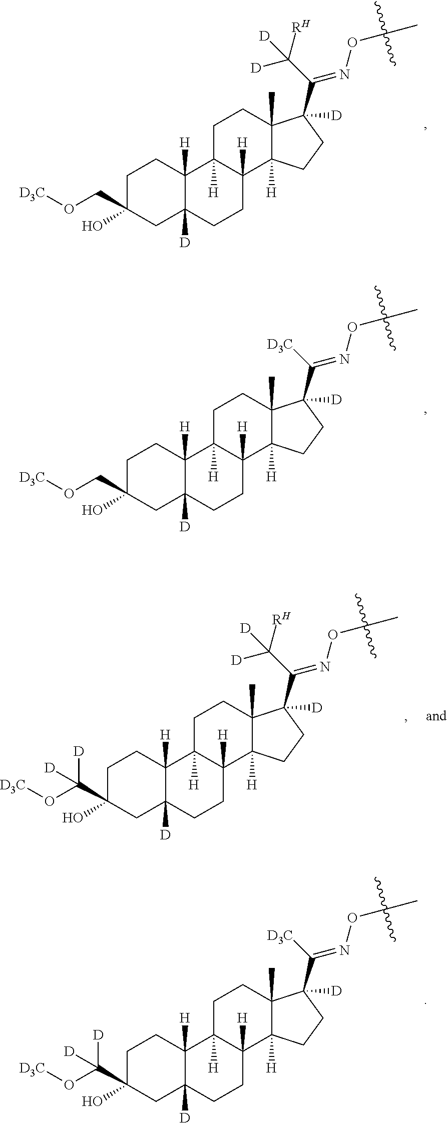

In some embodiments, the deuterated

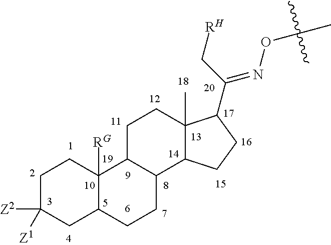

moiety is selected from:

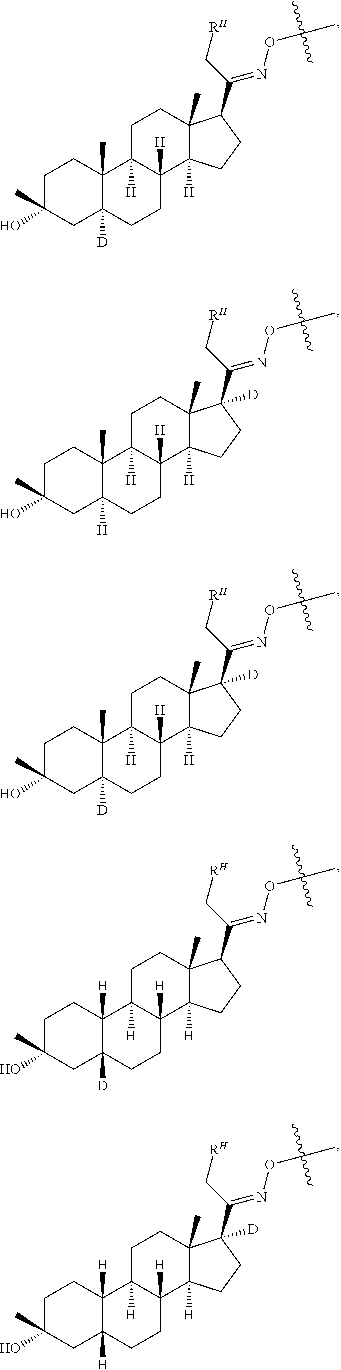

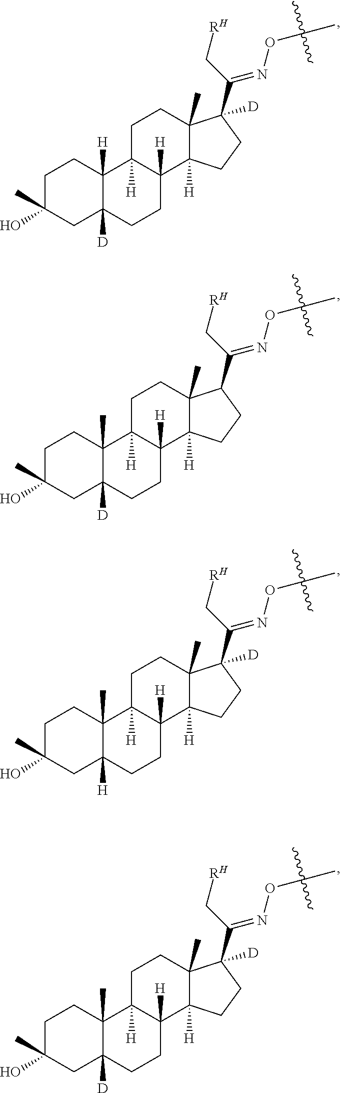

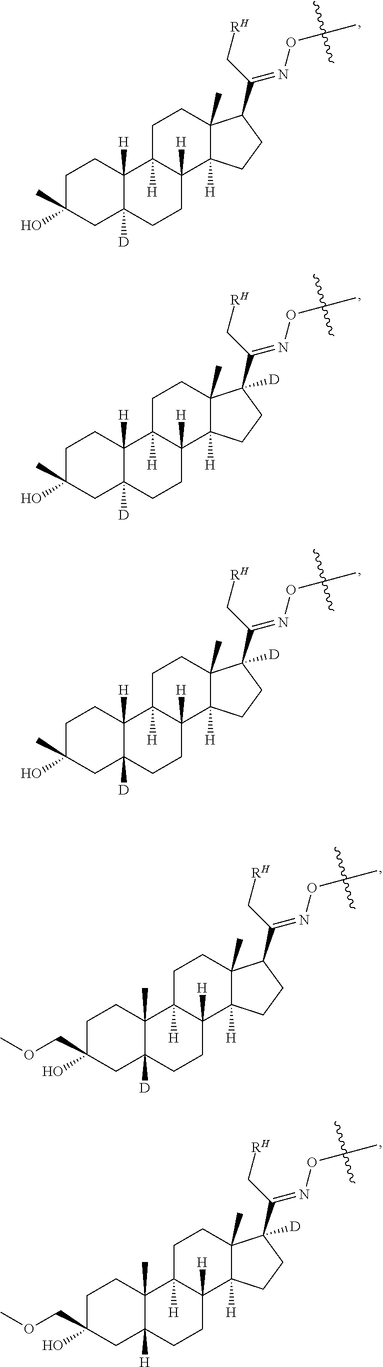

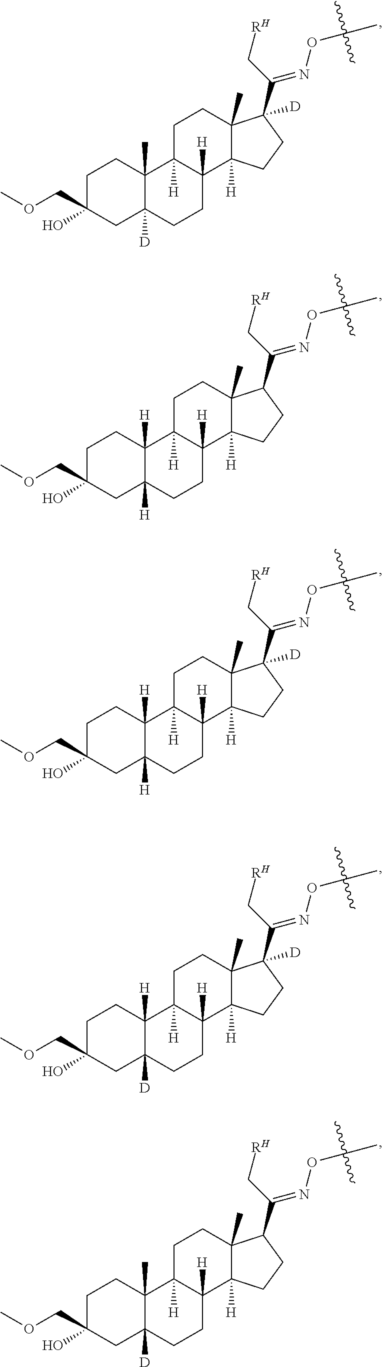

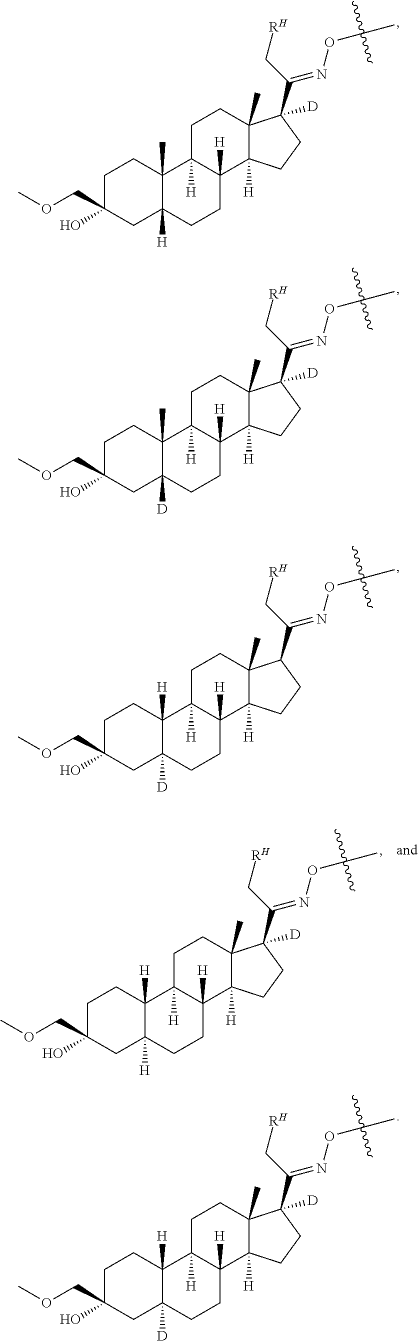

Consistent with their corresponding non-deuterated versions, the deuterated analogs described herein may be in a non-salt form or a salt form. For example, the deuterated analogs of the non-phosphate prodrugs may be in an HCl salt form. For example, the deuterated analogs of the phosphate prodrugs may be in a Tris or bis-Tris salt form.

### 2. Halogenated Analogs

This disclosure also provides halogenated analogs of (1) the neurosteroids described above in Sections II(A) of the Detailed Description and (2) the prodrugs of the neurosteroids described above in Sections II(B) and II(C) of the Detailed Description, wherein one or more non-ionizable hydrogen atoms in the

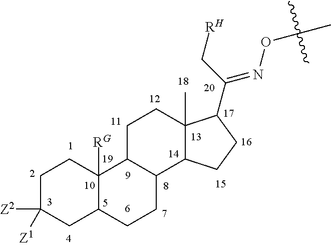

moiety of (1) the neurosteroids and (2) the prodrugs of the neurosteroids are replaced with halogen such as fluorine.

Consistent with their corresponding non-halogenated versions, the halogenated analogs may have a structure of Formula I and its sub-formulas (Formula I-1, Formula I-2), Formula II and its sub-formulas (Formula II-1, Formula II-2, Formula IIA, Formula IIA-1, Formula IIA-2, Formula IIB, Formula IIB-1, Formula IIB-2, Formula IIC, Formula IIC-1, Formula IIC-2, Formula IID, Formula IID-1, Formula IID-2), or Formula III and its sub-formulas (Formula III-1, Formula III-2), as described above, or a pharmaceutically acceptable salt, hydrate, or hydrated salt thereof.

In some embodiments, the halogenated

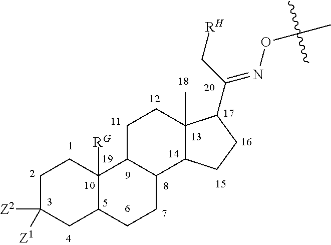

moiety is fully halogenated, i.e., all the non-ionizable hydrogen atoms in this moiety are replaced with halogen such as fluorine. In some embodiments, the halogenated

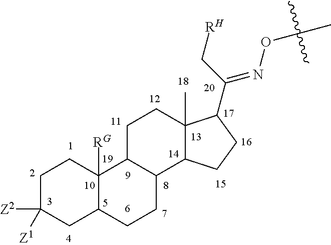

moiety is partially halogenated, i.e., one or more non-ionizable hydrogen atoms, but not all the non-ionizable hydrogen atoms, in this moiety are replaced with halogen such as fluorine.

In some embodiments, the halogenated analogs have enhanced metabolic stability. The enhanced metabolic stability may origin from an elevated energy barrier associated with cytochrome P450-mediated halogen abstraction, compared to hydrogen abstraction. Notably, the neurosteroids disclosed herein can be oxidatively modified at the C17 position (e.g., hydroxylation) and/or at the C4/C5/C6 position (e.g., formation of C4═C5 alkene or C5═C6 alkene) by cytochrome P450s. Accordingly, installation of halogen such as fluorine at one or more of these positions in the neurosteroids and the prodrugs of the neurosteroids can deter the oxidative modification events, thereby enhancing metabolic stability.

In some embodiments, the enhanced metabolic stability corresponds to a larger HLM t_{1/2}, RLM t_{1/2}, or MLM t_{1/2}. An exemplary method of measuring HLM t_{1/2}, RLM t_{1/2}, and MLM t_{1/2} is described in Example 8. In the context of the prodrugs, the HLM t_{1/2}, RLM t_{1/2}, and MLM t_{1/2} values refer to those determined using the corresponding neurosteroid parent compounds.

In some embodiments, the enhanced metabolic stability corresponds to a higher oral bioavailability in an animal model (such as mouse, rat, dog, or non-human primate) or human.

Methods of making exemplary halogenated analogs are disclosed in subsequent sections and exemplified by the Examples. The synthetic methods disclosed herein are compatible with a wide variety of functional groups and starting materials. Thus, a wide variety of halogenated analogs can be obtained from the disclosed methods.

In some embodiments, the hydrogen atom at the C17 position is replaced with halogen such as fluorine.

In some embodiments, the hydrogen atom at the C5 position is replaced with halogen such as fluorine.

In some embodiments, the two hydrogen atoms at the C4 position are replaced with halogen such as fluorine.

In some embodiments, the two hydrogen atoms at the C6 position are replaced with halogen such as fluorine.

In some embodiments, the two hydrogen atoms of the methylene group at the C20 position are replaced with halogen such as fluorine. In some embodiments, when R^{H} is hydrogen, the corresponding methyl group at the C20 position is fully halogenated (e.g., CF₃).

In some embodiments, Z² is halogenated, either fully halogenated or partially halogenated. In some embodiments, Z² is fluorinated, either fully fluorinated or partially fluorinated. In some embodiments, Z² is a fully halogenated methyl (e.g., —CF₃). In some embodiments, Z² is halogenated —CH₂OCH₃, such as —CF₂OCH₃, —CH₂OCF₃, or —CF₂OCF₃.

In some embodiments, the hydrogen atom at the C17 position is replaced with halogen such as fluorine, and the hydrogen atom at the C5 position is replaced with halogen such as fluorine.

In some embodiments, the hydrogen atom at the C17 position is replaced with halogen such as fluorine, and the two hydrogen atoms of the methylene group at the C20 position are replaced with halogen such as fluorine. In some embodiments, the hydrogen atom at the C17 position is replaced with halogen such as fluorine, and when R^{H} is hydrogen, the corresponding methyl group at the C20 position is fully halogenated (e.g., CF₃).

In some embodiments, the hydrogen atom at the C5 position is replaced with halogen such as fluorine, and Z² is halogenated, either fully halogenated or partially halogenated. In some embodiments, Z² is a fully halogenated methyl (e.g., —CF₃). In some embodiments, Z² is halogenated —CH₂OCH₃, such as —CF₂OCH₃, —CH₂OCF₃, or —CF₂OCF₃.

In some embodiments, the hydrogen atom at the C17 position is replaced with halogen such as fluorine, the hydrogen atom at the C5 position is replaced with halogen such as fluorine, and the methyl group at the C20 position is fully halogenated (e.g., CF₃).

In some embodiments, the hydrogen atom at the C17 position is replaced with halogen such as fluorine, the hydrogen atom at the C5 position is replaced with halogen such as fluorine, and Z² is halogenated, either fully halogenated or partially halogenated. In some embodiments, Z² is a fully halogenated methyl (e.g., —CF₃). In some embodiments, Z² is halogenated —CH₂OCH₃, such as —CF₂OCH₃, —CH₂OCF₃, or —CF₂OCF₃.

In some embodiments, the hydrogen atom at the C17 position is replaced with halogen such as fluorine, the hydrogen atom at the C5 position is replaced with halogen such as fluorine, the two hydrogen atoms of the methylene group at the C20 position are replaced with halogen such as fluorine, and Z² is halogenated, either fully halogenated or partially halogenated. In some embodiments, the hydrogen atom at the C17 position is replaced with halogen such as fluorine; the hydrogen atom at the C5 position is replaced with halogen such as fluorine; when R^{H} is hydrogen, the corresponding methyl group at the C20 position is fully halogenated (e.g., CF₃); and Z² is halogenated, either fully halogenated or partially halogenated. In some embodiments, Z² is a fully halogenated methyl (e.g., —CF₃). In some embodiments, Z² is halogenated —CH₂OCH₃, such as —CF₂OCH₃, —CH₂OCF₃, or —CF₂OCF₃.

In some embodiments, the halogenated

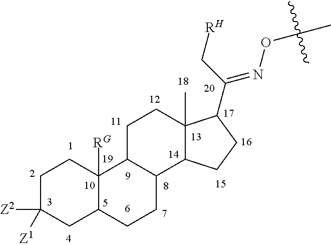

moiety is selected from:

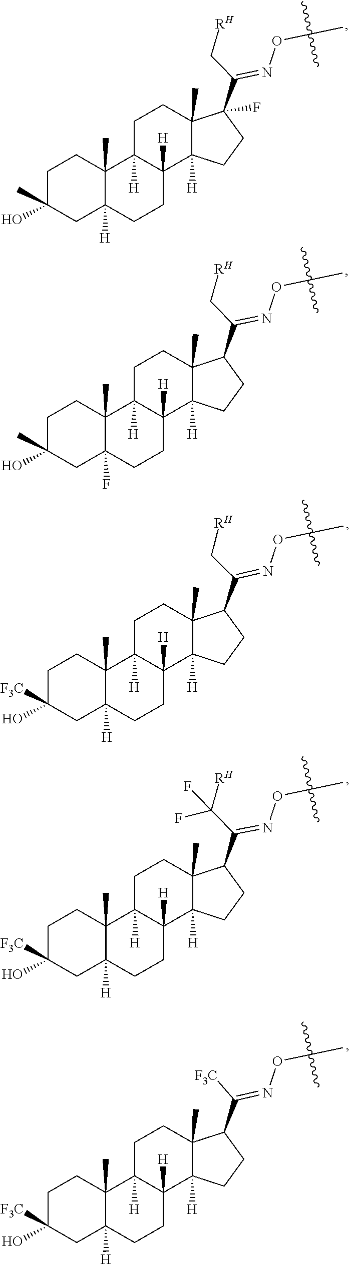

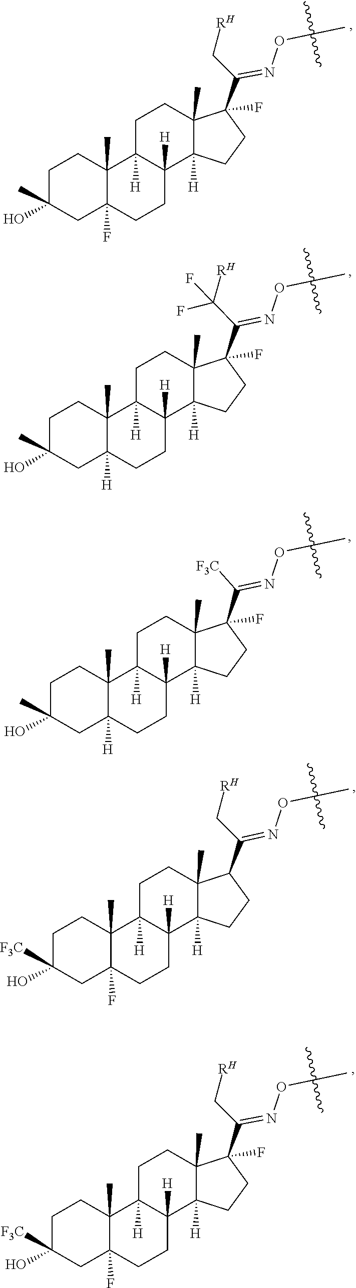

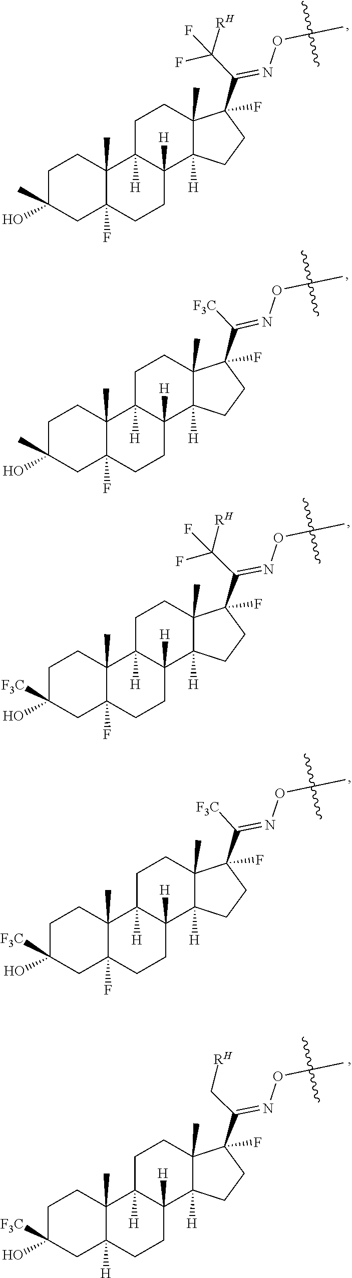

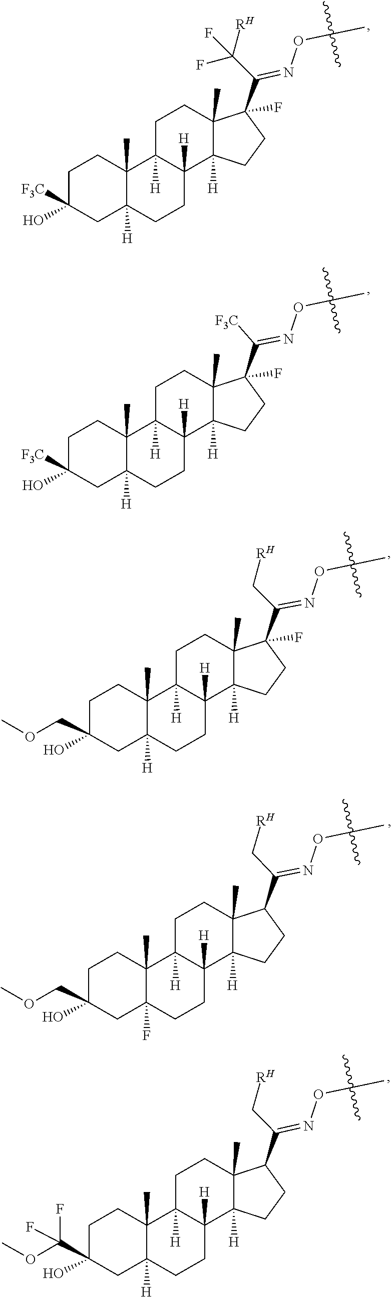

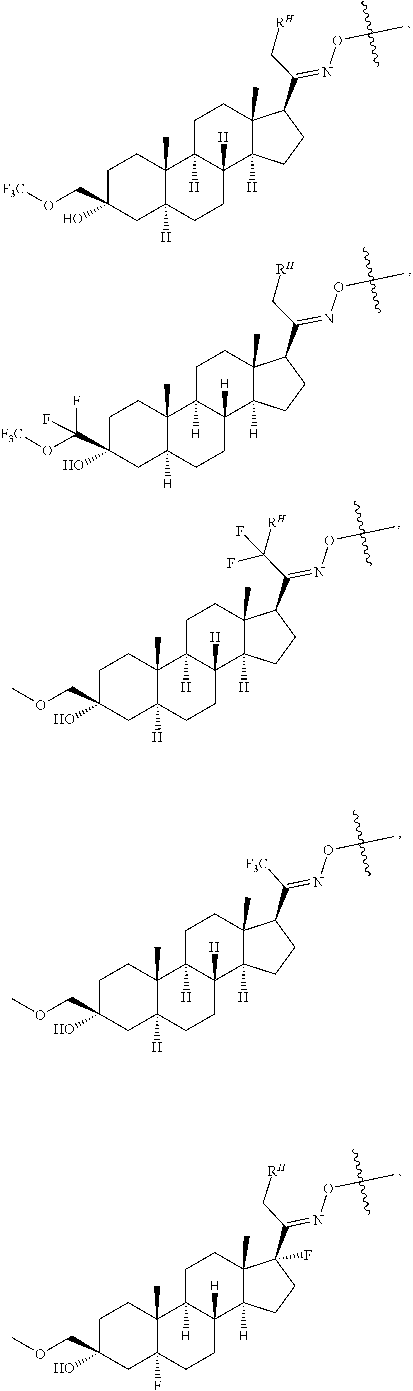

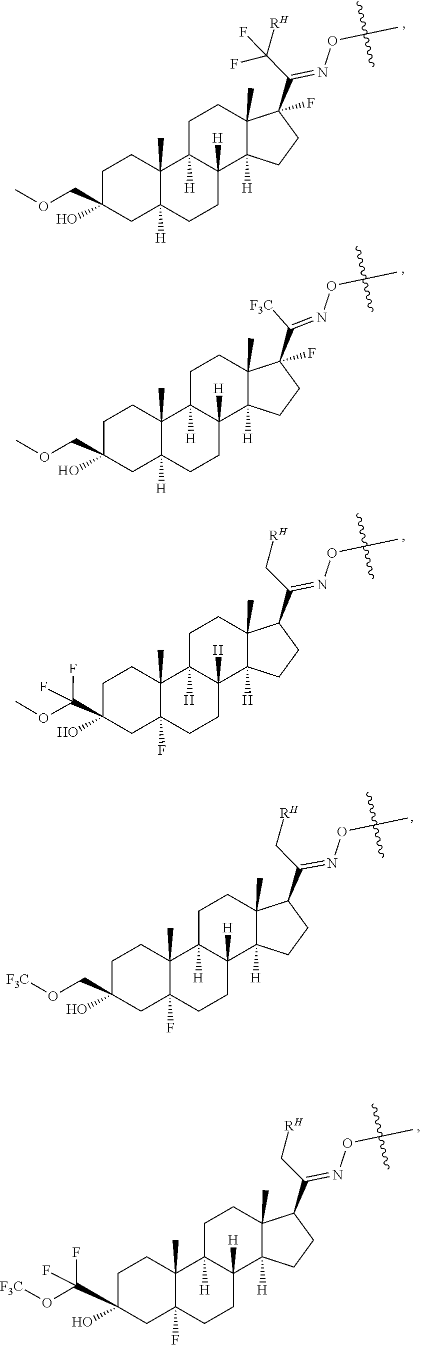

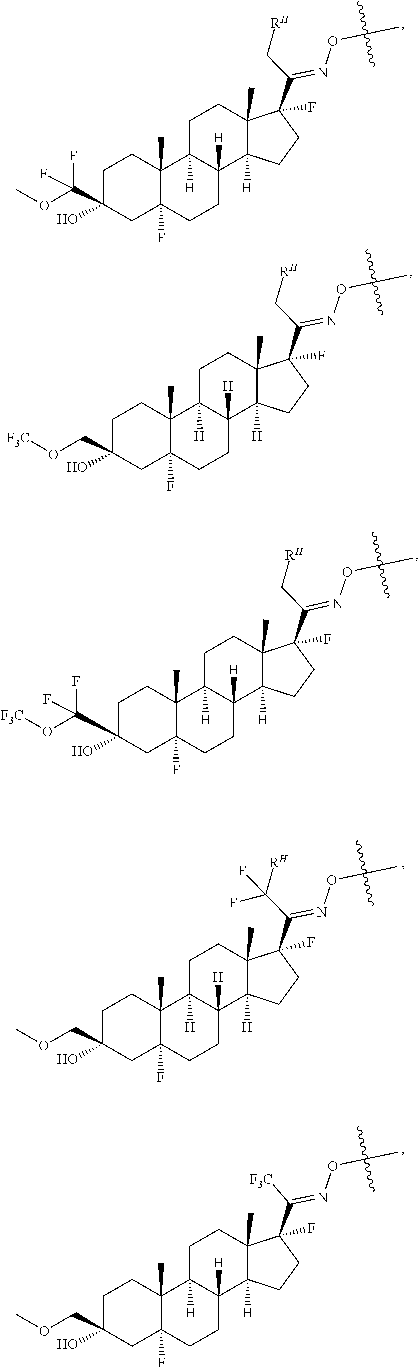

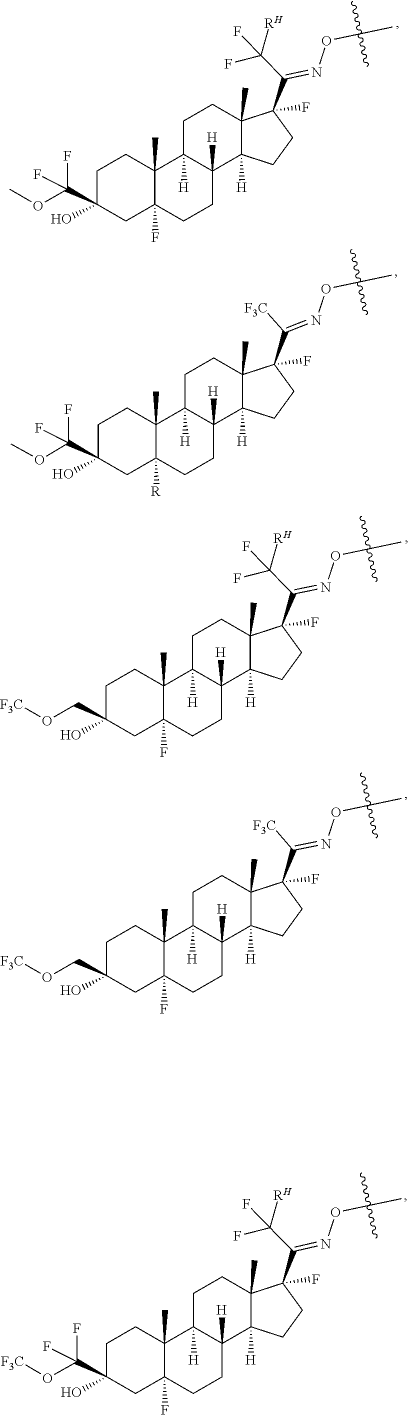

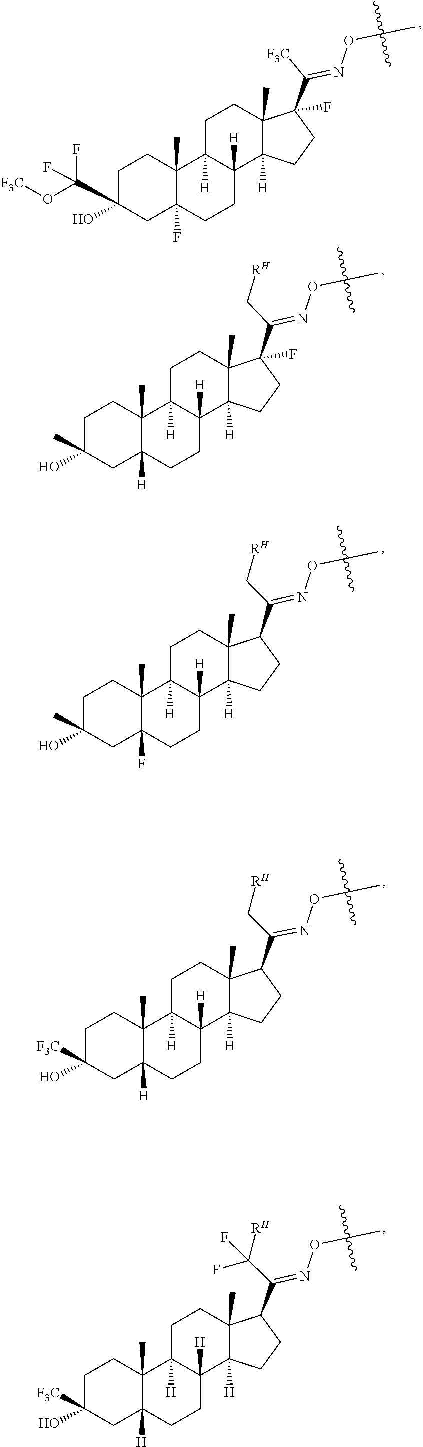

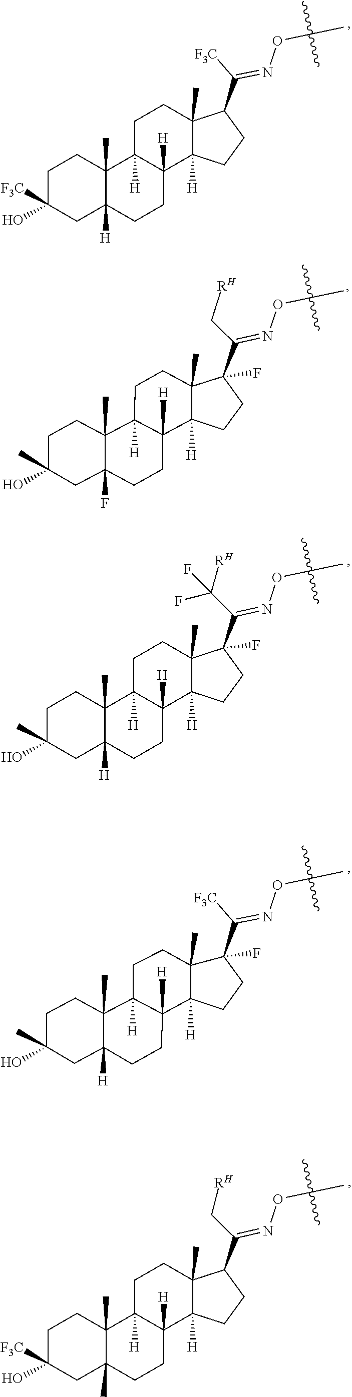

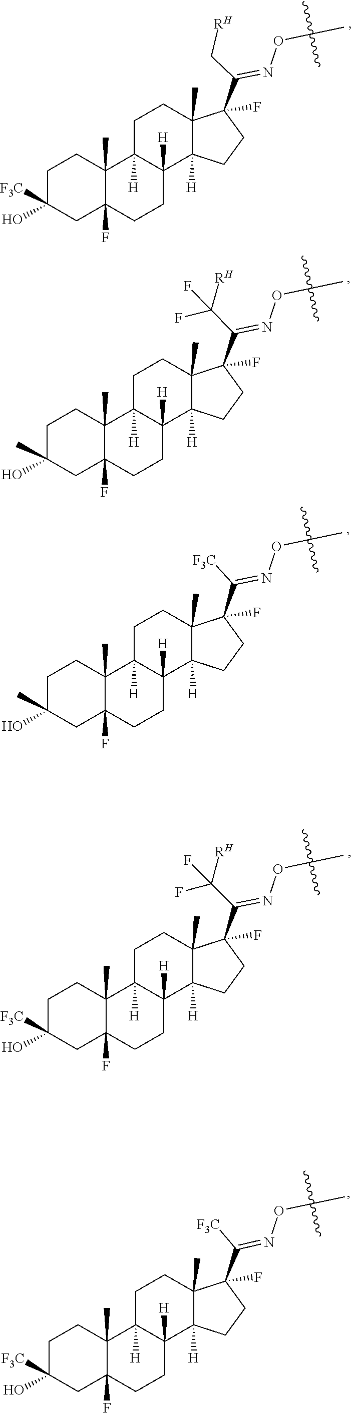

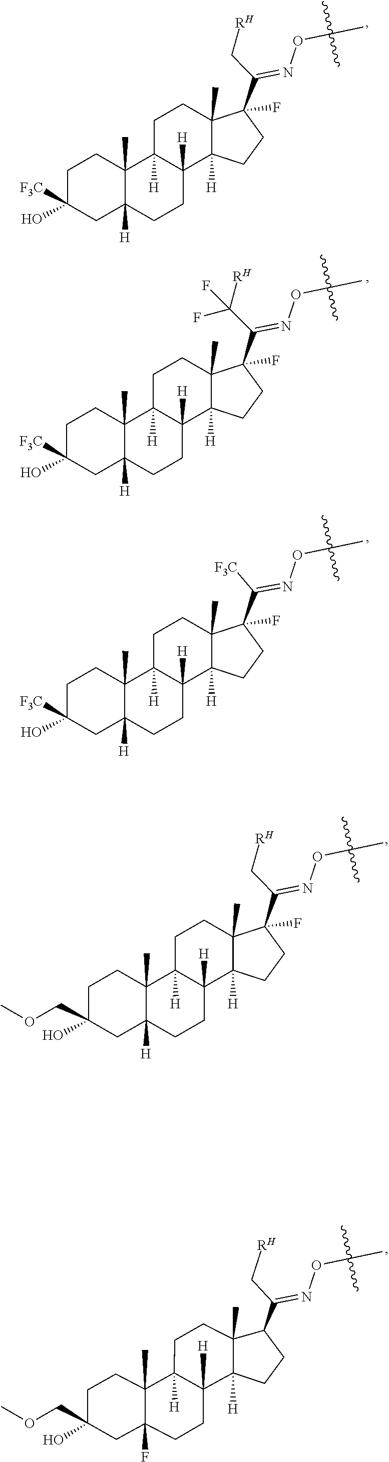

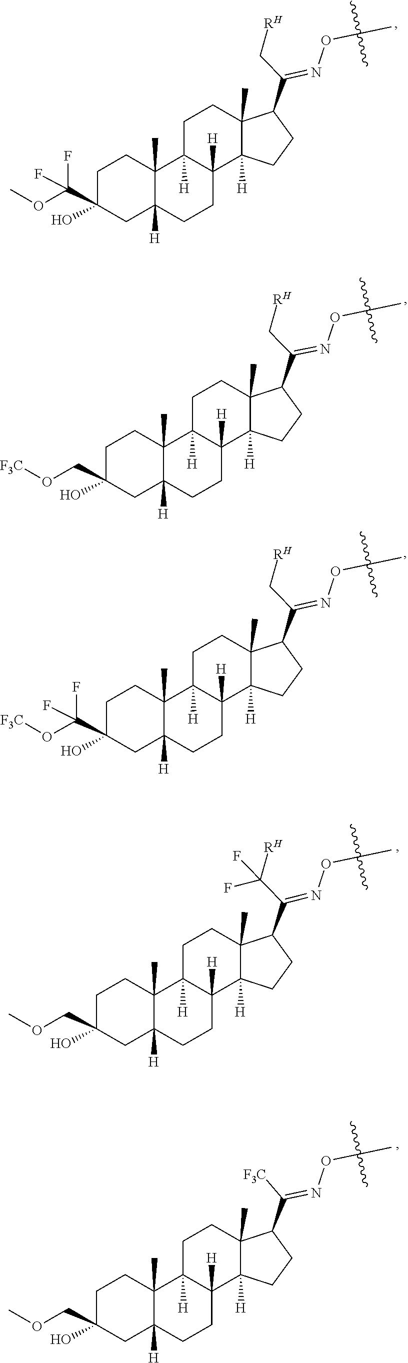

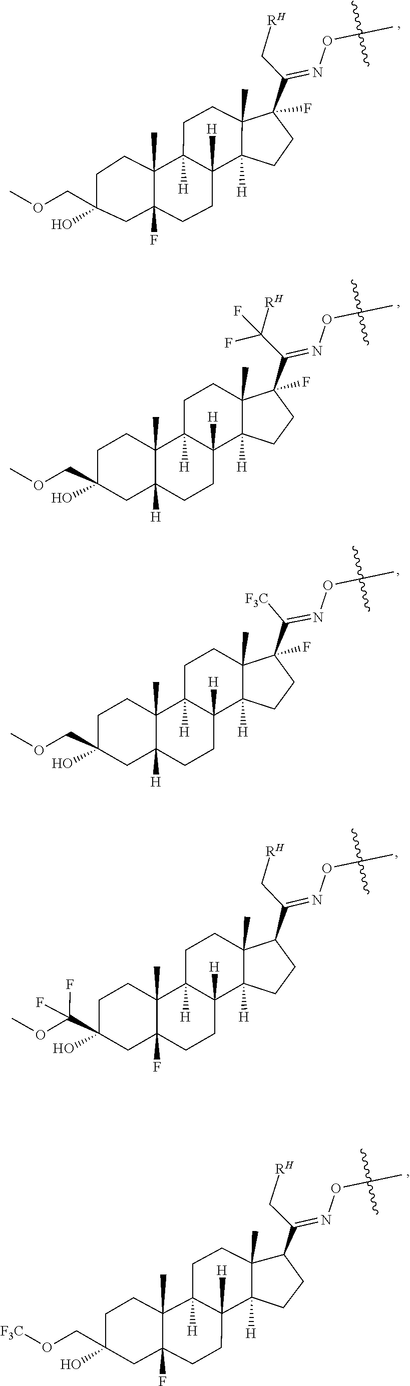

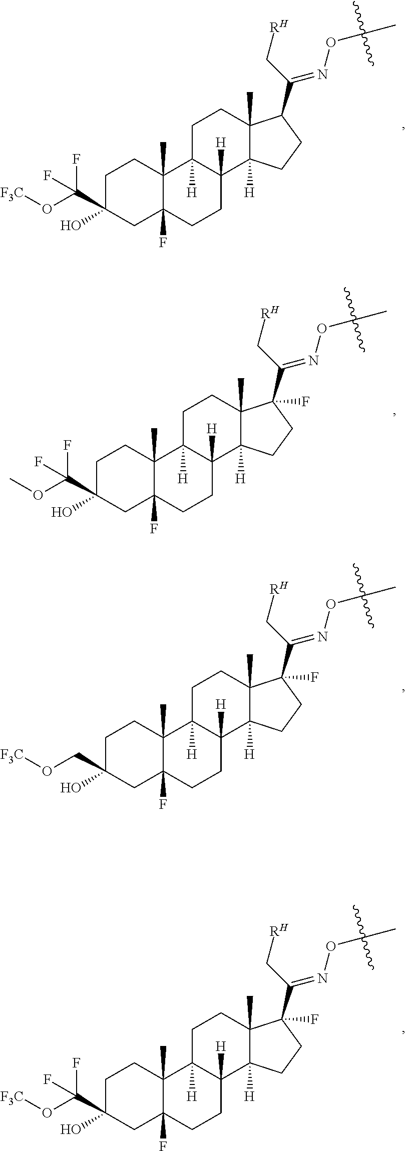

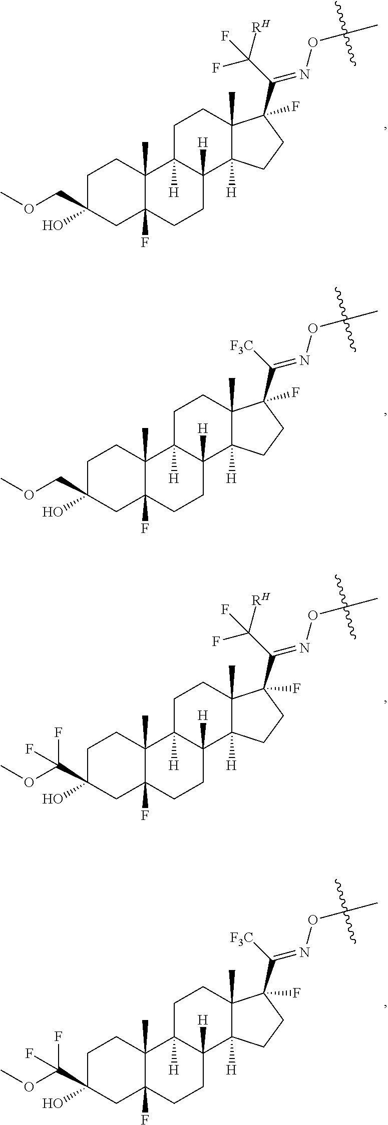

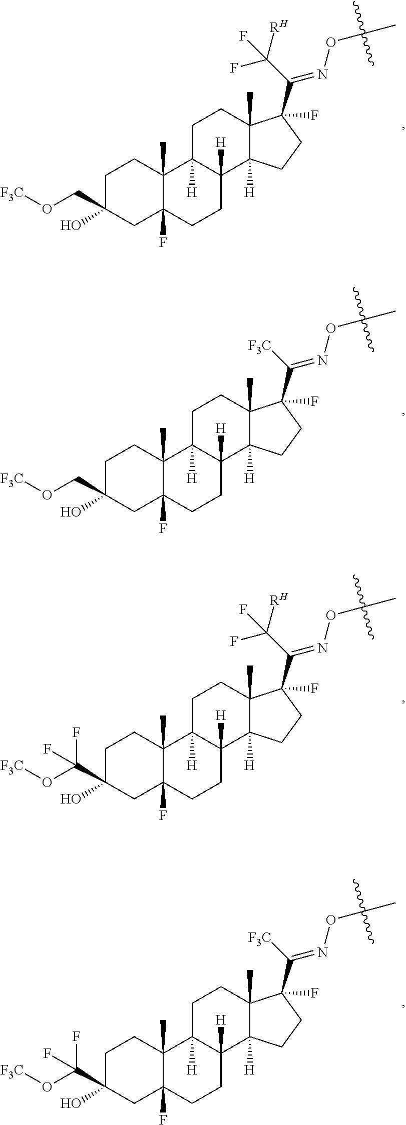

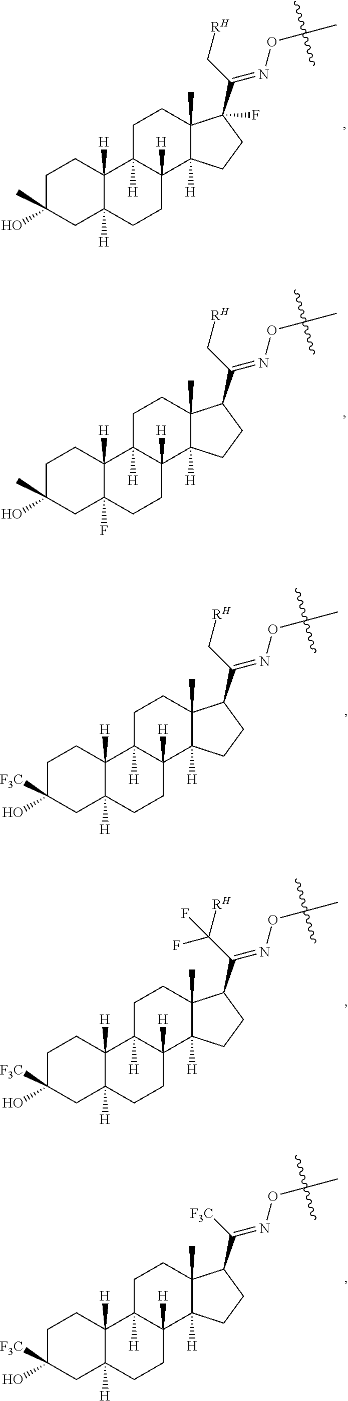

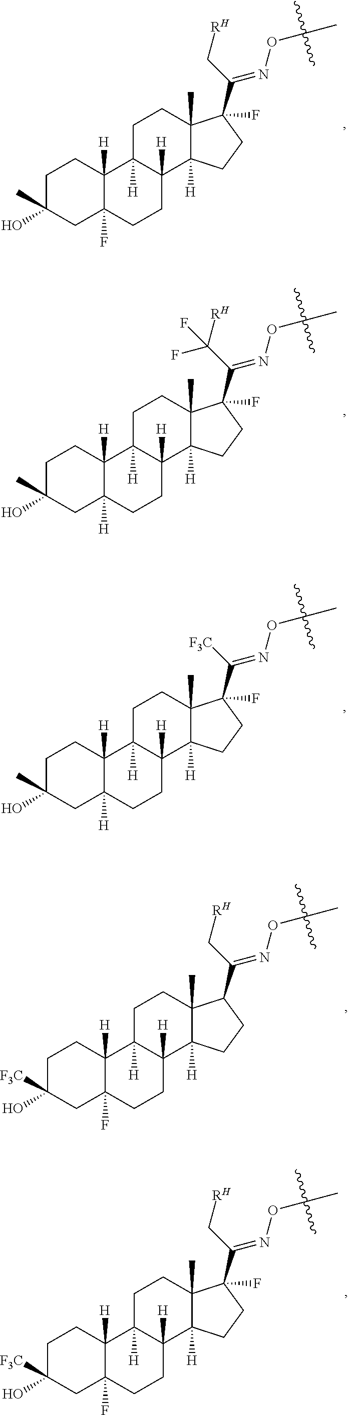

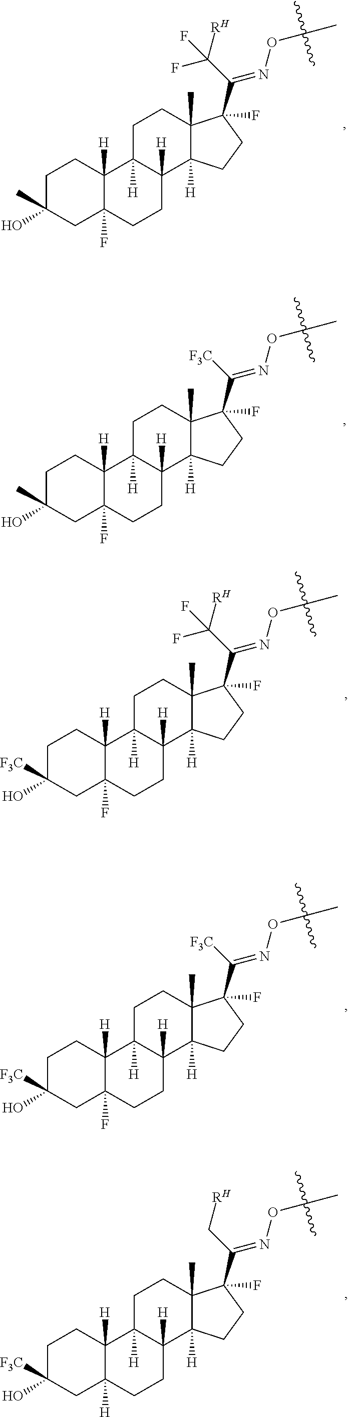

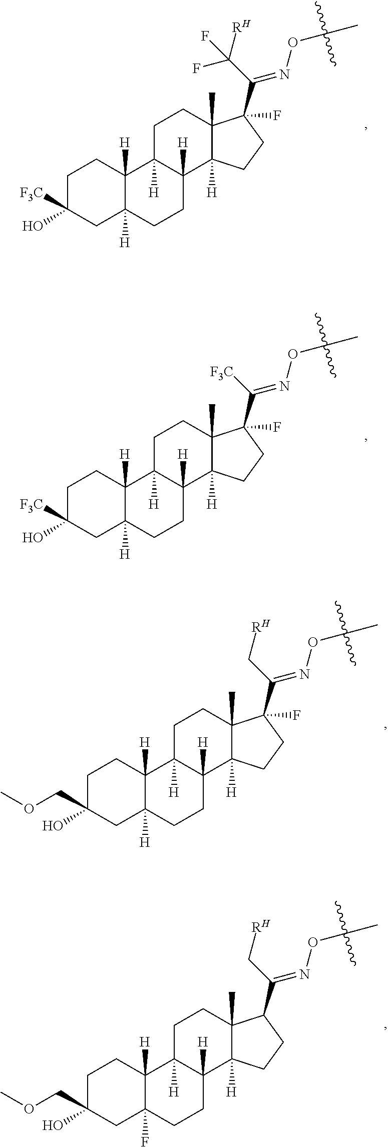

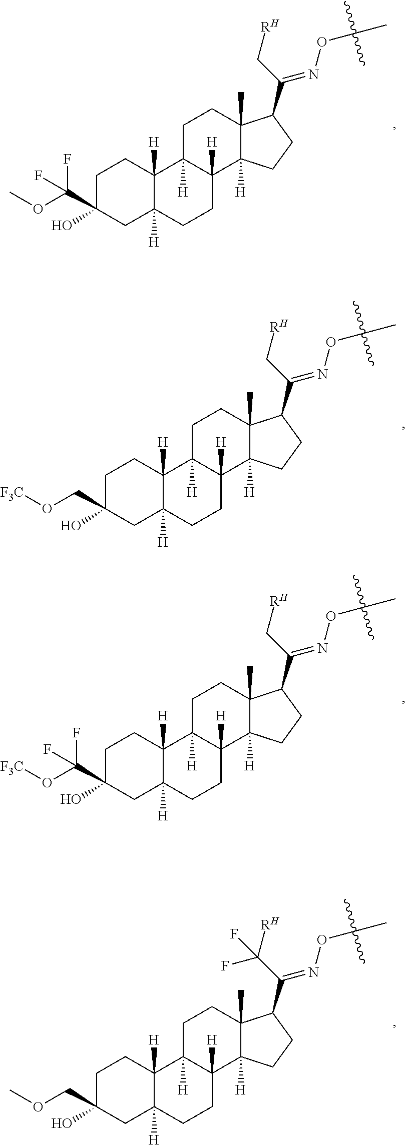

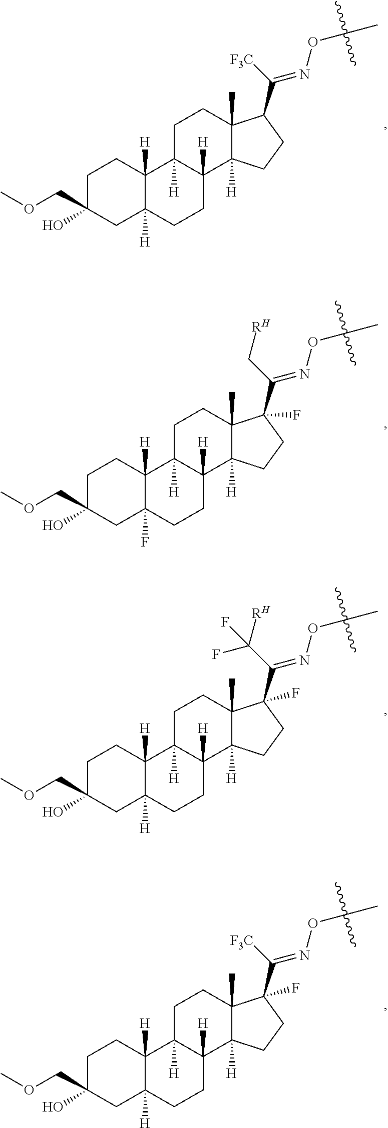

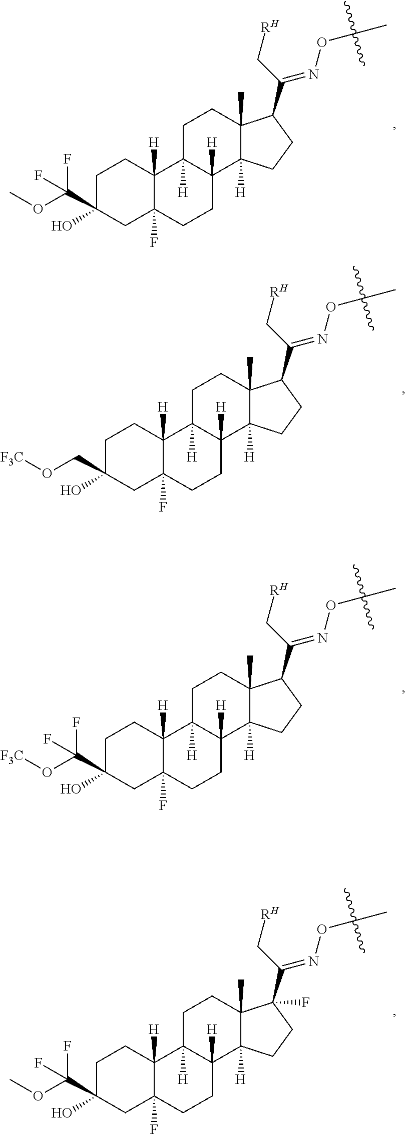

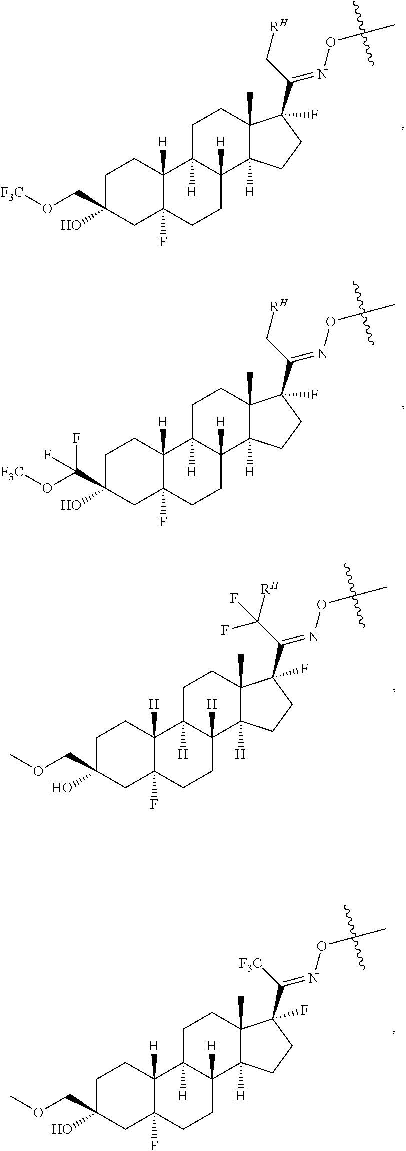

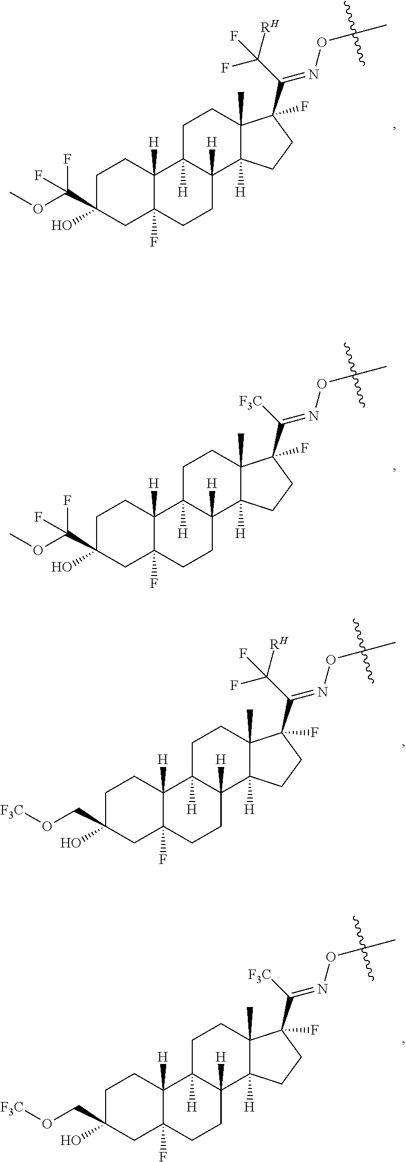

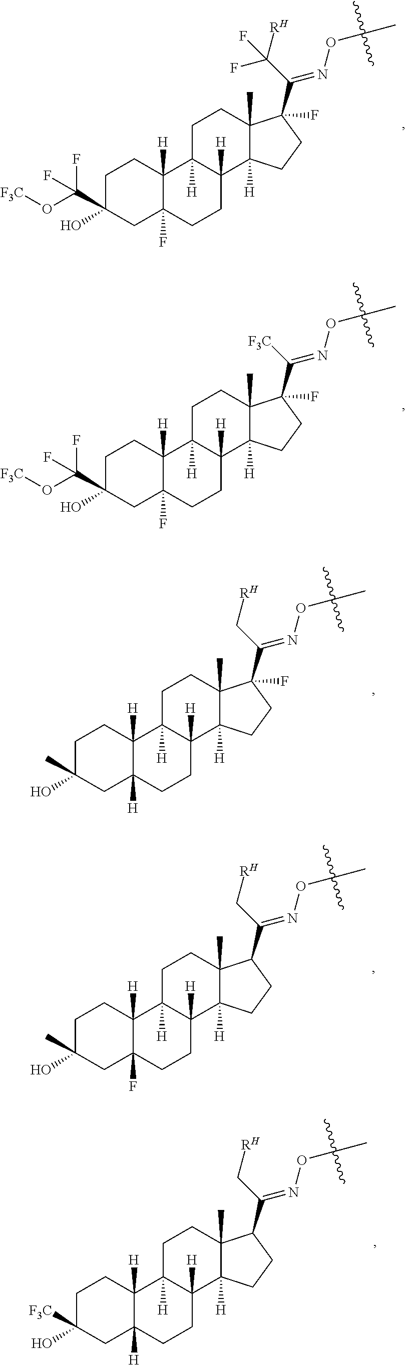

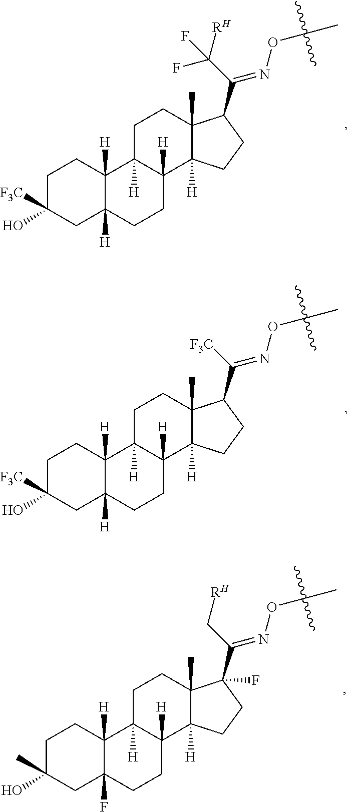

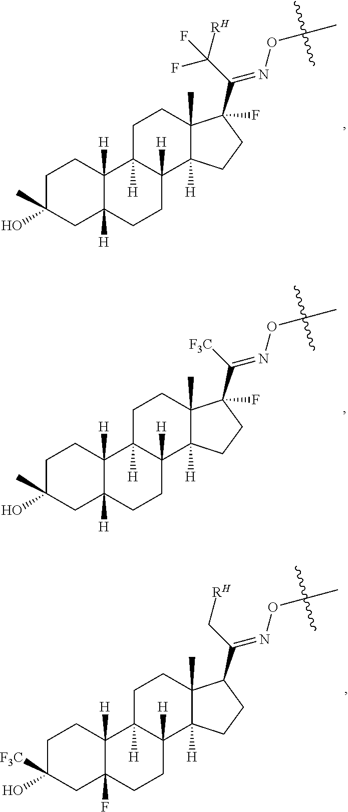

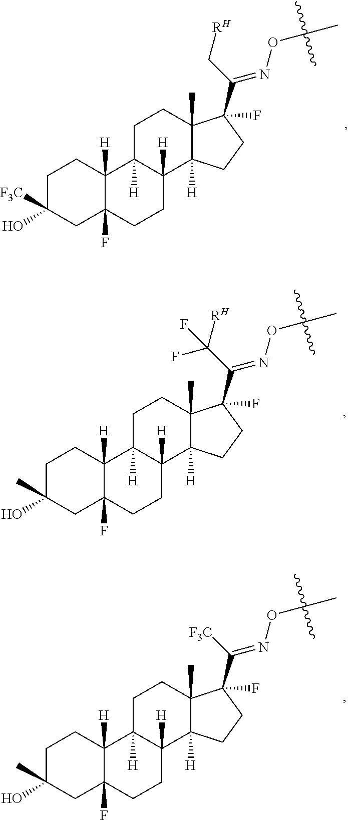

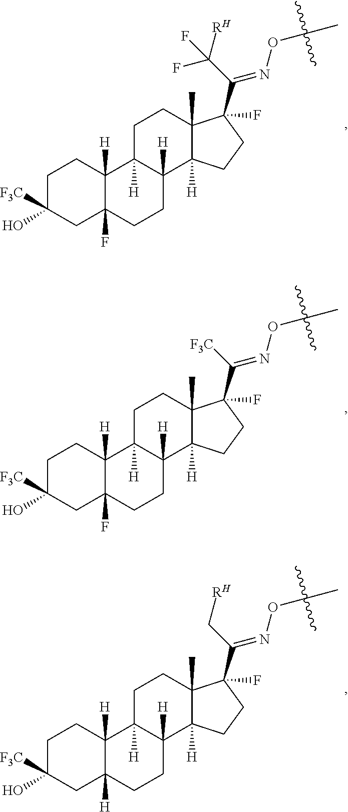

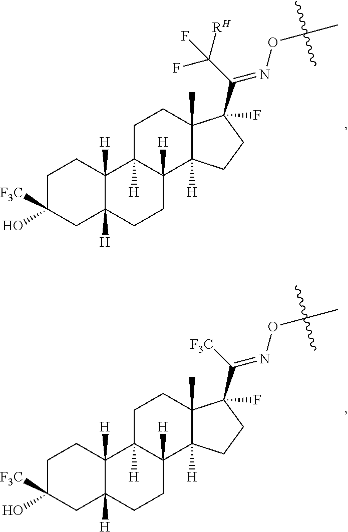

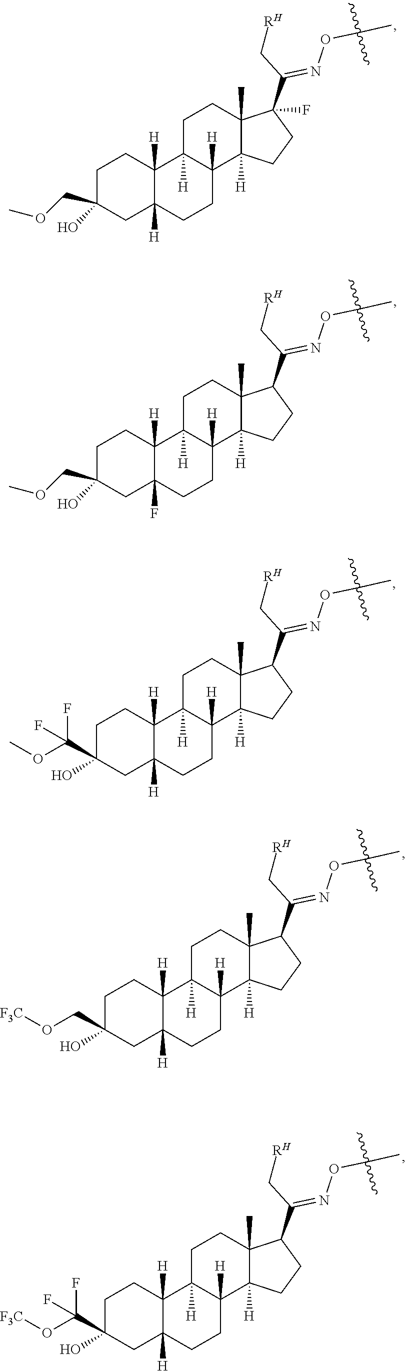

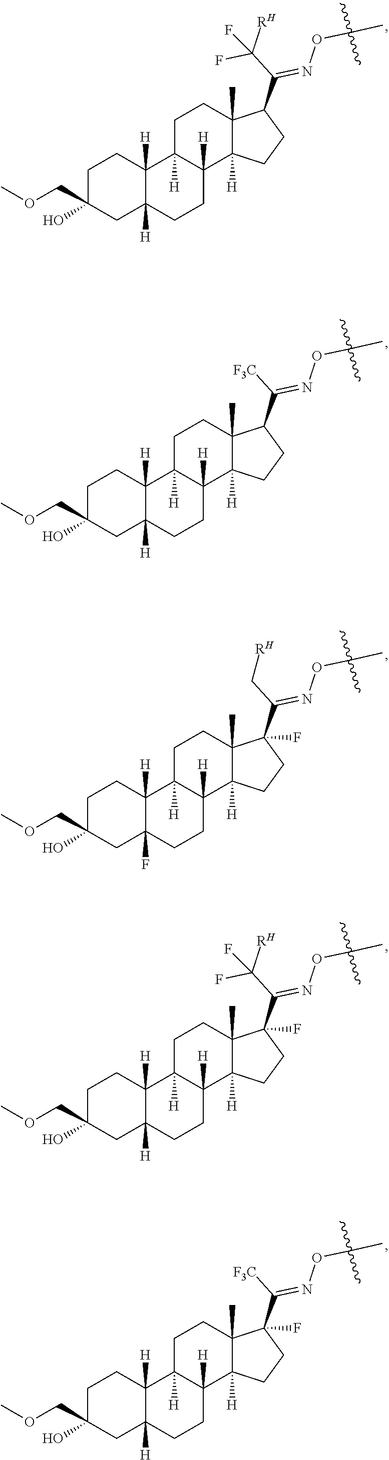

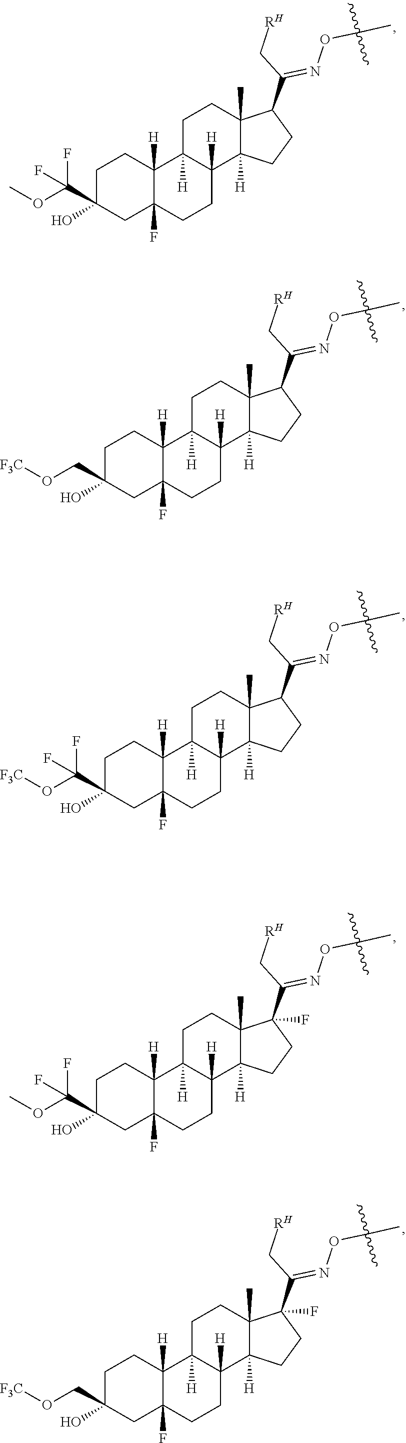

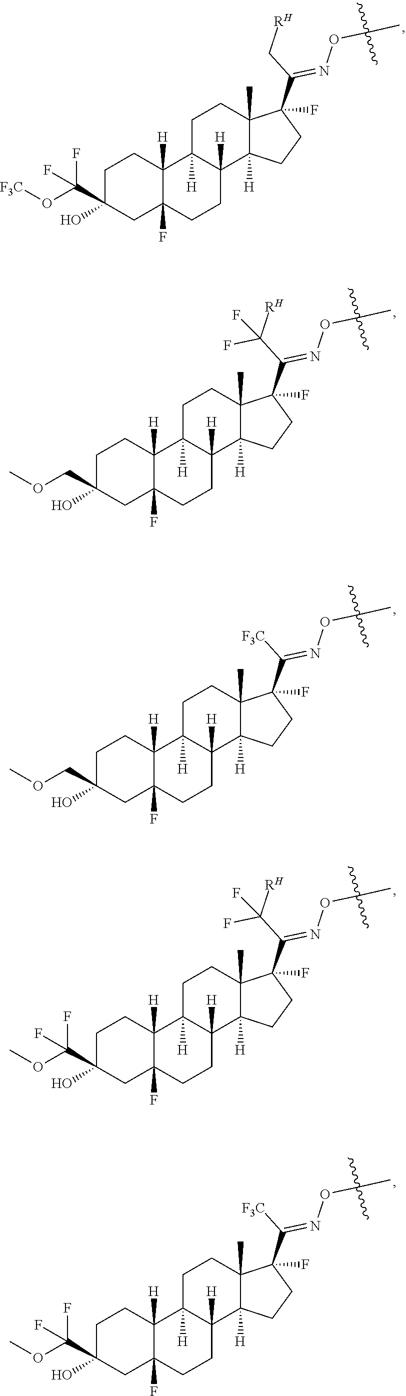

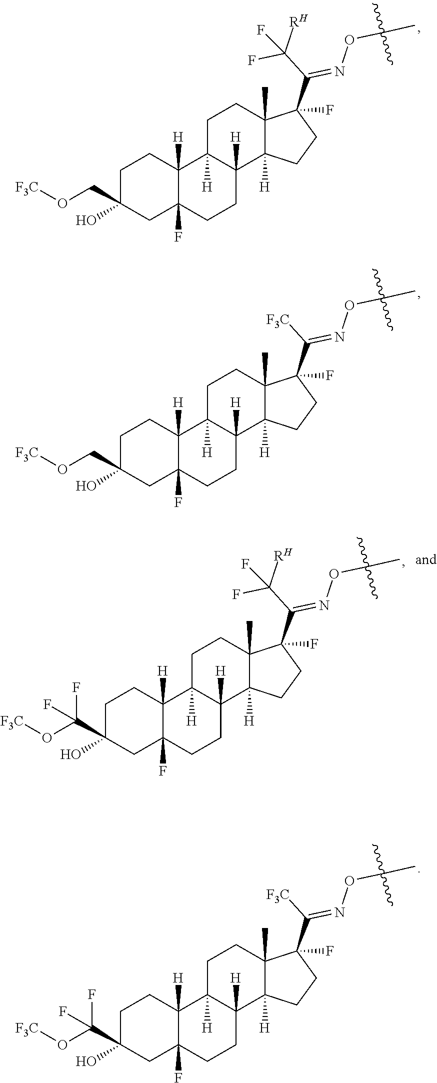

In some embodiments, the halogenated

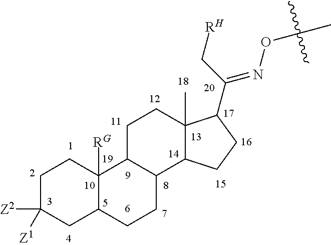

moiety is selected from:

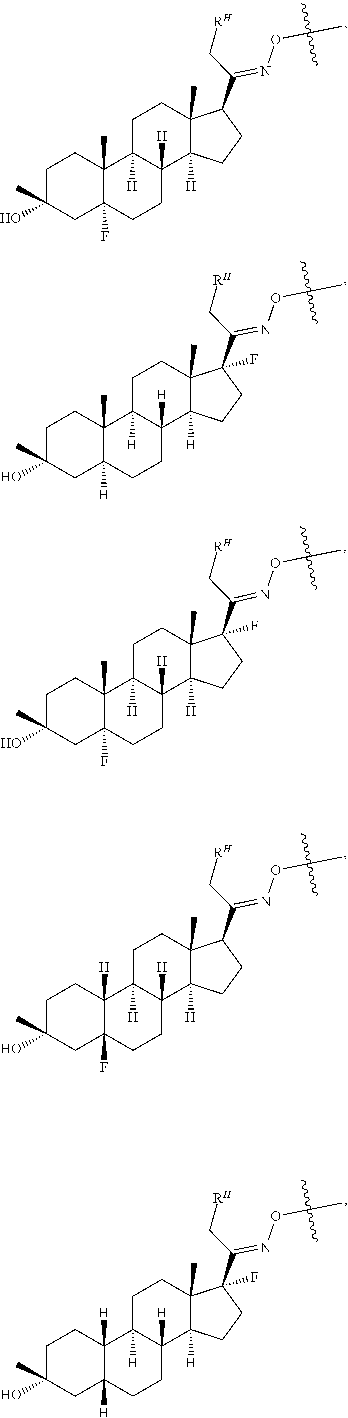

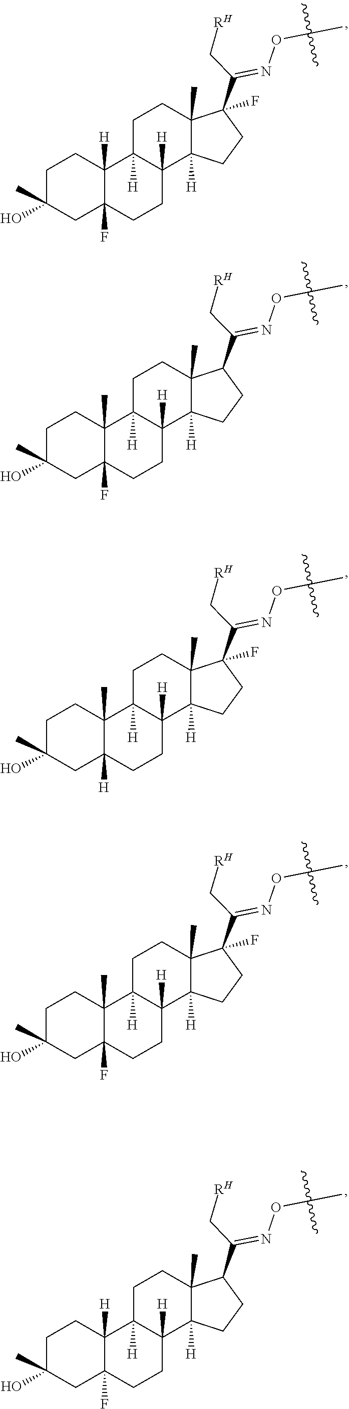

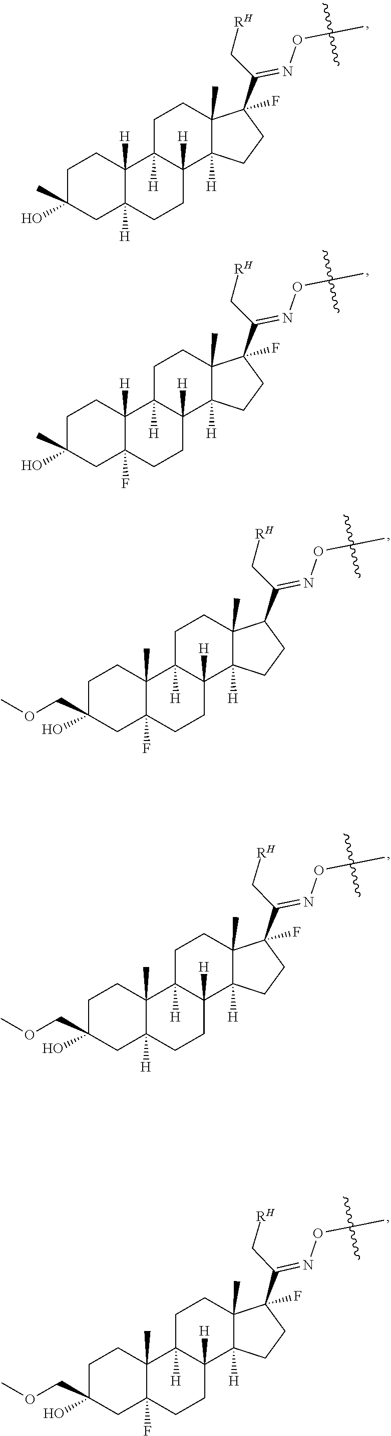

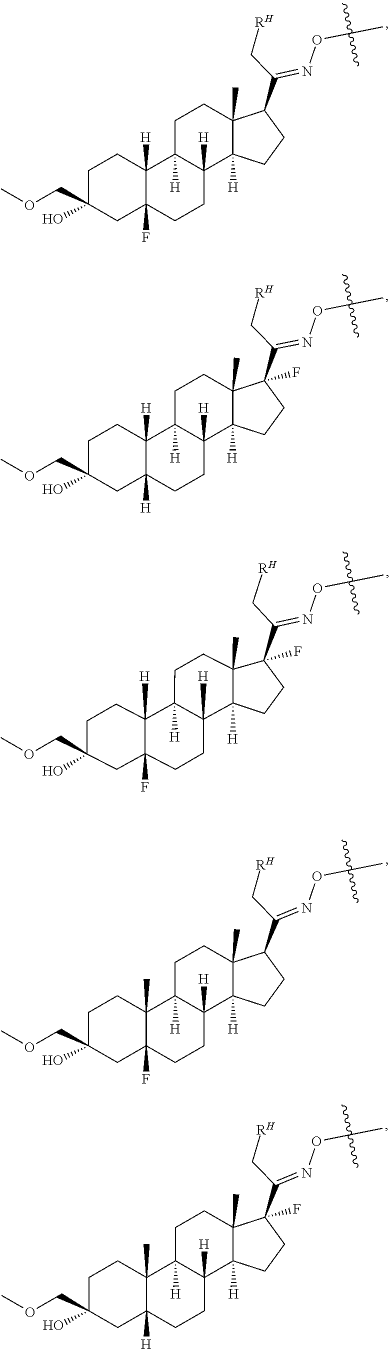

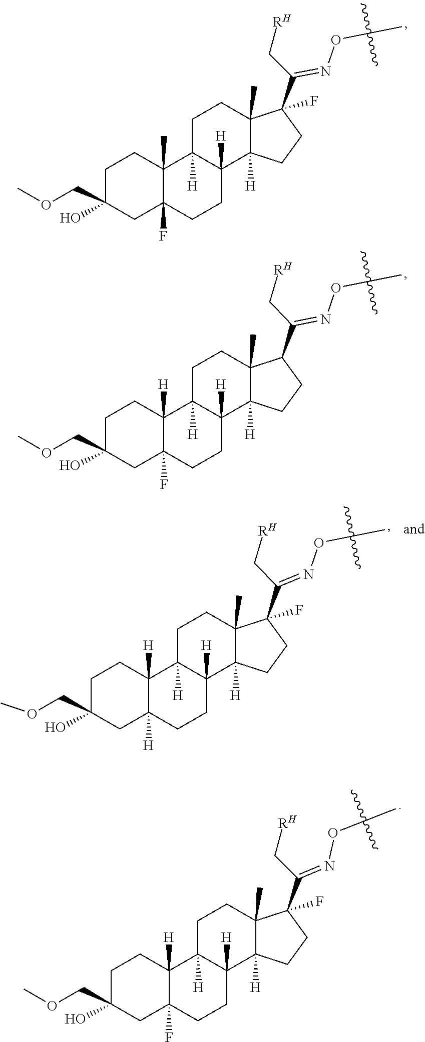

Consistent with their corresponding non-halogenated versions, the halogenated analogs described herein may be in a non-salt form or a salt form. For example, the halogenated analogs of the non-phosphate prodrugs may be in an HCl salt form. For example, the halogenated analogs of the phosphate prodrugs may be in a Tris or bis-Tris salt form.

### III. Compositions

Disclosed are compositions containing a compound disclosed herein. In this context, the compound may be a neurosteroid described herein, a prodrug described herein, or a deuterated or halogenated analog described herein. In some embodiments, the compound in the composition is in greater than 80%, 85%, 90%, or 95% enantiomeric or diastereomeric excess. In some embodiments, the compound in the composition is in greater than 95% enantiomeric or diastereomeric excess.

In some embodiments, the compositions contain a compound having a structure of Formula I or a pharmaceutically acceptable salt, hydrate, or hydrated salt of Formula I, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula I-1 or a pharmaceutically acceptable salt, hydrate, or hydrated salt of Formula I-1, wherein the compound is in greater than 80%, 85%, 90%, or 95% enantiomeric or diastereomeric excess for the configuration depicted by Formula I-1. In some embodiments, the compound is in greater than 95% enantiomeric or diastereomeric excess for the configuration depicted by Formula I-1.

In some embodiments, the compositions contain a compound having a structure of Formula I-2 or a pharmaceutically acceptable salt, hydrate, or hydrated salt of Formula I-2, wherein the compound is in greater than 80%, 85%, 90%, or 95% enantiomeric or diastereomeric excess for the configuration depicted by Formula I-2. In some embodiments, the compound is in greater than 95% enantiomeric or diastereomeric excess for the configuration depicted by Formula I-2.

In some embodiments, the compositions contain a compound having a structure of Formula II or a pharmaceutically acceptable salt, hydrate, or hydrated salt of Formula II, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula II-1 or a pharmaceutically acceptable salt, hydrate, or hydrated salt of Formula II-1, wherein the compound is in greater than 80%, 85%, 90%, or 95% enantiomeric or diastereomeric excess for the configuration depicted by Formula II-1. In some embodiments, the compound is in greater than 95% enantiomeric or diastereomeric excess for the configuration depicted by Formula II-1.

In some embodiments, the compositions contain a compound having a structure of Formula II-2 or a pharmaceutically acceptable salt, hydrate, or hydrated salt of Formula II-2, wherein the compound is in greater than 80%, 85%, 90%, or 95% enantiomeric or diastereomeric excess for the configuration depicted by Formula II-2. In some embodiments, the compound is in greater than 95% enantiomeric or diastereomeric excess for the configuration depicted by Formula II-2.

In some embodiments, the compositions contain a compound having a structure of Formula IIA or a pharmaceutically acceptable salt, hydrate, or hydrated salt of Formula IIA, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula IIA-1 or a pharmaceutically acceptable salt, hydrate, or hydrated salt of Formula IIA-1, wherein the compound is in greater than 80%, 85%, 90%, or 95% enantiomeric or diastereomeric excess for the configuration depicted by Formula IIA-1. In some embodiments, the compound is in greater than 95% enantiomeric or diastereomeric excess for the configuration depicted by Formula IIA-1.

In some embodiments, the compositions contain a compound having a structure of Formula IIA-2 or a pharmaceutically acceptable salt, hydrate, or hydrated salt of Formula IIA-2, wherein the compound is in greater than 80%, 85%, 90%, or 95% enantiomeric or diastereomeric excess for the configuration depicted by Formula IIA-2. In some embodiments, the compound is in greater than 95% enantiomeric or diastereomeric excess for the configuration depicted by Formula IIA-2.

In some embodiments, the compositions contain a compound having a structure of Formula IIB or a pharmaceutically acceptable salt, hydrate, or hydrated salt of Formula IIB, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula IIB-1 or a pharmaceutically acceptable salt, hydrate, or hydrated salt of Formula IIB-1, wherein the compound is in greater than 80%, 85%, 90%, or 95% enantiomeric or diastereomeric excess for the configuration depicted by Formula IIB-1. In some embodiments, the compound is in greater than 95% enantiomeric or diastereomeric excess for the configuration depicted by Formula IIB-1.

In some embodiments, the compositions contain a compound having a structure of Formula IIB-2 or a pharmaceutically acceptable salt, hydrate, or hydrated salt of Formula IIB-2, wherein the compound is in greater than 80%, 85%, 90%, or 95% enantiomeric or diastereomeric excess for the configuration depicted by Formula IIB-2. In some embodiments, the compound is in greater than 95% enantiomeric or diastereomeric excess for the configuration depicted by Formula IIB-2.

In some embodiments, the compositions contain a compound having a structure of Formula IIC or a pharmaceutically acceptable salt, hydrate, or hydrated salt of Formula IIC, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula IIC-1 or a pharmaceutically acceptable salt, hydrate, or hydrated salt of Formula IIC-1, wherein the compound is in greater than 80%, 85%, 90%, or 95% enantiomeric or diastereomeric excess for the configuration depicted by Formula ITC-1. In some embodiments, the compound is in greater than 95% enantiomeric or diastereomeric excess for the configuration depicted by Formula IIC-1.

In some embodiments, the compositions contain a compound having a structure of Formula IIC-2 or a pharmaceutically acceptable salt, hydrate, or hydrated salt of Formula IIC-2, wherein the compound is in greater than 80%, 85%, 90%, or 95% enantiomeric or diastereomeric excess for the configuration depicted by Formula IIC-2. In some embodiments, the compound is in greater than 95% enantiomeric or diastereomeric excess for the configuration depicted by Formula IIC-2.

In some embodiments, the compositions contain a compound having a structure of Formula IID or a pharmaceutically acceptable salt, hydrate, or hydrated salt of Formula IID, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula IID-1 or a pharmaceutically acceptable salt, hydrate, or hydrated salt of Formula IID-1, wherein the compound is in greater than 80%, 85%, 90%, or 95% enantiomeric or diastereomeric excess for the configuration depicted by Formula IID-1. In some embodiments, the compound is in greater than 95% enantiomeric or diastereomeric excess for the configuration depicted by Formula IID-1.

In some embodiments, the compositions contain a compound having a structure of Formula IID-2 or a pharmaceutically acceptable salt, hydrate, or hydrated salt of Formula IID-2, wherein the compound is in greater than 80%, 85%, 90%, or 95% enantiomeric or diastereomeric excess for the configuration depicted by Formula IID-2. In some embodiments, the compound is in greater than 95% enantiomeric or diastereomeric excess for the configuration depicted by Formula IID-2.

In some embodiments, the compositions contain a compound having a structure of Formula III or a pharmaceutically acceptable salt, hydrate, or hydrated salt of Formula III, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula III-1 or a pharmaceutically acceptable salt, hydrate, or hydrated salt of Formula III-1, wherein the compound is in greater than 80%, 85%, 90%, or 95% enantiomeric or diastereomeric excess for the configuration depicted by Formula III-1. In some embodiments, the compound is in greater than 95% enantiomeric or diastereomeric excess for the configuration depicted by Formula III-1.

In some embodiments, the compositions contain a compound having a structure of Formula III-2 or a pharmaceutically acceptable salt, hydrate, or hydrated salt of Formula III-2, wherein the compound is in greater than 80%, 85%, 90%, or 95% enantiomeric or diastereomeric excess for the configuration depicted by Formula III-2. In some embodiments, the compound is in greater than 95% enantiomeric or diastereomeric excess for the configuration depicted by Formula III-2.

The disclosed compounds may be present in a mixture of a salt form and a non-salt form. In some embodiments, more than 50%, 60%, 70%, 80%, 90%, 95%, or 98% of the compound in the mixture may be in the non-salt form, calculated as the ratio of the weight of the non-salt form to the total weight of the mixture. In some embodiments, more than 90% of the compound in the mixture may be in the non-salt form. In some embodiments, more than 50%, 60%, 70%, 80%, 90%, 95%, or 98% of the compound in the mixture may be in the salt form, calculated as the ratio of the weight of the salt form to the total weight of the mixture. In some embodiments, more than 90% of the compound in the mixture may be in the salt form. In some embodiments, more than 50%, 60%, 70%, 80%, 90%, 95%, or 98% of the compound in the mixture may be in an HCl salt form, calculated as the ratio of the weight of the HCl salt form to the total weight of the mixture. In some embodiments, more than 90% of the compound in the mixture may be in the HCl salt form. In some embodiments, more than 50%, 60%, 70%, 80%, 90%, 95%, or 98% of the compound in the mixture may be in a Tris salt form, calculated as the ratio of the weight of the Tris salt form to the total weight of the mixture. In some embodiments, more than 90% of the compound in the mixture may be in the Tris salt form. In some embodiments, more than 50%, 60%, 70%, 80%, 90%, 95%, or 98% of the compound in the mixture may be in a bis-Tris salt form, calculated as the ratio of the weight of the bis-Tris salt form to the total weight of the mixture. In some embodiments, more than 90% of the compound in the mixture may be in the bis-Tris salt form.

### IV. Formulations

Disclosed are pharmaceutical formulations containing a compound or composition described herein. Generally, the pharmaceutical formulations also contain one or more pharmaceutically acceptable excipients.

The pharmaceutical formulations can be in a form chosen from tablets, capsules, caplets, pills, powders, beads, granules, particles, creams, gels, solutions (such as aqueous solutions, e.g., buffer, saline, and buffered saline), emulsions, suspensions (including nano- and micro-suspensions), nanoparticulate formulations, etc. In some embodiments, the pharmaceutical formulations are formulated for oral administration. In some embodiments, the pharmaceutical formulations are formulated for intravenous administration. In some embodiments, the pharmaceutical formulations are formulated for intramuscular administration.

In some embodiments, the pharmaceutical formulations are in the form of tablets, caplets, capsules, or pills. In some embodiment, the tablets, caplets, capsules, and pills have an enteric coating to prevent the gastric acids in the stomach from dissolving or degrading the active ingredients. Exemplary enteric coatings are known in the art and described in the sections below.

As used herein, “emulsion” refers to a mixture of non-miscible components homogenously blended together. In some forms, the non-miscible components include a lipophilic component and an aqueous component. For example, an emulsion may be a preparation of one liquid distributed in small globules throughout the body of a second liquid. The dispersed liquid is the discontinuous phase, and the dispersion medium is the continuous phase. When oil or an oleaginous substance is the dispersed liquid and water or an aqueous solution is the continuous phase, it is known as an oil-in-water emulsion, whereas when water or an aqueous solution is the dispersed phase and oil or an oleaginous substance is the continuous phase, it is known as a water-in-oil emulsion.

As used herein, “biocompatible” refers to materials that are neither themselves toxic to the host (e.g., a non-human animal or human), nor degrade (if the material degrades) at a rate that produces monomeric or oligomeric subunits or other byproducts at toxic concentrations in the host.

As used herein, “biodegradable” refers to degradation or breakdown of a polymeric material into smaller (e.g., non-polymeric) subunits or digestion of the material into smaller subunits.

As used herein, “enteric polymers” refers to polymers that become soluble in the higher pH environment of the lower gastrointestinal tract or slowly erode as they pass through the gastrointestinal tract.

As used herein, “nanoparticulate formulations” generally refers to formulations containing nanoparticles, which are particles having a diameter from about 1 nm to about 1000 nm, from about 10 nm to about 1000 nm, from about 100 nm to about 1000 nm, or from about 250 nm to about 1000 nm. In some embodiments, “nanoparticulate formulations” can also refer to formulations containing microparticles, which are particles having a diameter from about 1 micron to about 100 microns, from about 1 to about 50 microns, from about 1 to about 30 microns, from about 1 micron to about 10 microns. In some embodiments, the nanoparticulate formulation may contain a mixture of nanoparticles, as defined above, and microparticles, as defined above.

As used herein, “surfactant” refers to any agent which preferentially absorbs to an interface between two immiscible phases, such as the interface between water (or aqueous solution) and an organic solvent (or organic solution), between water (or aqueous solution) and air, or between organic solvent (or organic solution) and air. Surfactants generally possess a hydrophilic moiety and a lipophilic moiety.

As used herein, “gel” is a semisolid system containing a dispersion of the active ingredient, i.e., a compound or composition according to the present disclosure, in a liquid vehicle that is rendered semisolid by the action of a thickening agent or polymeric material dissolved or suspended in the liquid vehicle. The liquid vehicle may include a lipophilic component, an aqueous component or both.

As used herein, “hydrogel” refers to a swollen, water-containing network of finely dispersed polymer chains that are water-insoluble, where the polymer molecules are in the external or dispersion phase and water (or an aqueous solution) forms the internal or dispersed phase. The polymer chains can be chemically cross-linked (chemical gels) or physically cross-linked (physical gels). Chemical gels possess polymer chains connected through covalent bonds, whereas physical gels have polymer chains linked by non-covalent interactions, such as van der Waals interactions, ionic interactions, hydrogen bonding interactions, and hydrophobic interactions.

As used herein, “beads” refers to beads made with the active ingredient (i.e., a compound or composition according to the present disclosure) and one or more pharmaceutically acceptable excipients. The beads can be produced by applying the active ingredient to an inert support, e.g., inert sugar core coated with the active ingredient. Alternatively, the beads can be produced by creating a “core” comprising both the active ingredient and at least one of the one or more pharmaceutically acceptable excipients. As used herein, “granules” refers to a product made by processing particles of the active ingredient (i.e., a compound or composition according to the present disclosure) that may or may not include one or more pharmaceutical acceptable excipients. Typically, granules do not contain an inert support and are bigger in size compared to the particles used to produce them. Although beads, granules and particles may be formulated to provide immediate release, beads and granules are usually employed to provide delayed release.

As used herein, “enzymatically degradable polymers” refers to polymers that are degraded by bacterial enzymes present in the intestines and/or lower gastrointestinal tract.

### A. Physical Forms and Unit Dosages

Depending upon the administration route, the compounds or compositions described herein may be formulated in a variety of ways. The pharmaceutical formulations can be prepared in various forms, such as tablets, capsules, caplets, pills, granules, powders, nanoparticle formulations, solutions (such as aqueous solutions, e.g., buffer, saline, and buffered saline), suspensions (including nano- and micro-suspensions), emulsions, creams, gels, and the like.

In some embodiments, the pharmaceutical formulations are in a solid dosage form suitable for simple administration of precise dosages. For example, the solid dosage form may be selected from tablets, soft or hard gelatin or non-gelatin capsules, and caplets for oral administration. In some embodiments, the solid dosage form is hard gelatin capsules. Optionally, the solid dosage form is a lyophilized powder that can be readily dissolved and converted to a liquid dosage form for intravenous or intramuscular administration. In some embodiments, the lyophilized powder is manufactured by dissolving the active ingredient (i.e., a compound or composition disclosed herein) in an aqueous medium followed by lyophilization. In some embodiments, the aqueous medium is water, normal saline, PBS, or an acidic aqueous medium such as an acetate buffer.

In some embodiments, the pharmaceutical formulations are in a liquid dosage form suitable for intravenous or intramuscular administration. Exemplary liquid dosage forms include, but are not limited to, solutions, suspensions, and emulsions. In some embodiments, the pharmaceutical formulations are in the form of a sterile aqueous solution. In some embodiments, the sterile aqueous solution is a sterile normal saline solution. In some embodiments, the sterile aqueous solution is a sterile PBS solution. In some embodiments, the sterile aqueous solution is an acidic, sterile aqueous solution such as a sterile acetate buffer. In some embodiments, the sterile aqueous solution is manufactured by dissolving a lyophilized powder containing the active ingredient (i.e., a compound or composition disclosed herein) in an aqueous medium. For example, the sterile aqueous solution can be prepared by dissolving the lyophilized powder containing the active ingredient in a dose-appropriate volume of sterile water, sterile normal saline, sterile PBS, or acidic, sterile aqueous medium such as a sterile acetate buffer. In some embodiments, the lyophilized powder containing the active ingredient is the same as those described in the paragraph above.

In some embodiments, the pharmaceutical formulations are in a unit dosage form, and may be suitably packaged, for example, in a box, blister, vial, bottle, syringe, sachet, ampoule, or in any other suitable single-dose or multi-dose holder or container, optionally with one or more leaflets containing product information and/or instructions for use.

### B. Pharmaceutically Acceptable Excipients

Exemplary pharmaceutically acceptable excipients include, but are not limited to, diluents, binders, lubricants, disintegrants, pH-modifying or buffering agents, salts (such as NaCl), preservatives, antioxidants, solubility enhancers, wetting or emulsifying agents, plasticizers, colorants (such as pigments and dyes), flavoring or sweetening agents, thickening agents, emollients, humectants, stabilizers, glidants, solvents or dispersion mediums, surfactants, pore formers, and coating or matrix materials.

In some embodiments, the powders described herein, including the lyophilized powders, contain one or more of the following pharmaceutically acceptable excipients: pH-modifying or buffering agents, salts (such as NaCl), and preservatives.

In some embodiments, the tablets, beads, granules, and particles described herein contain one or more of the following pharmaceutically acceptable excipients: coating or matrix materials, diluents, binders, lubricants, disintegrants, pigments, stabilizers, and surfactants. If desired, the tablets, beads, granules, and particles may also contain a minor amount of nontoxic auxiliary substances such as wetting or emulsifying agents, dyes, pH-buffering agents, and preservatives.

Examples of the coating or matrix materials include, but are not limited to, cellulose polymers (such as methylcellulose, ethyl cellulose, cellulose acetate, cellulose acetate phthalate, hydroxypropyl cellulose, hydroxyethyl cellulose, hydroxymethyl cellulose, hydroxypropyl methylcellulose, hydroxypropyl methylcellulose phthalate, hydroxypropyl methylcellulose acetate succinate, cellulose acetate trimellitate, and carboxymethylcellulose sodium), vinyl polymers and copolymers (such as polyvinyl pyrrolidone, polyvinyl acetate, polyvinyl acetate phthalate, vinyl acetate-crotonic acid copolymer, and ethylene-vinyl acetate copolymer), acrylic acid polymers and copolymers (such as those formed from acrylic acid, methacrylic acid, methyl acrylate, ethyl acrylate, methyl methacrylate, or ethyl methacrylate, as well as methacrylic resins that are commercially available under the tradename EUDRAGIT®), enzymatically degradable polymers (such as azo polymers, pectin, chitosan, amylose, and guar gum), zein, shellac, and polysaccharides. In some embodiments, the coating or matrix materials may contain one or more excipients such as plasticizers, colorants, glidants, stabilizers, pore formers, and surfactants.

In some embodiments, the coating or matrix materials are pH-sensitive or pH-responsive polymers, such as the enteric polymers commercially available under the tradename EUDRAGIT®. For example, EUDRAGIT® L30D-55 and L100-55 are soluble at pH 5.5 and above; EUDRAGIT® L100 is soluble at pH 6.0 and above; EUDRAGIT® S is soluble at pH 7.0 and above.

In some embodiments, the coating or matrix materials are water-insoluble polymers having different degrees of permeability and expandability, such as EUDRAGIT® NE, RL, and RS.

Depending on the coating or matrix materials, the decomposition/degradation or structural change of the pharmaceutical formulations may occur at different locations of the gastrointestinal tract. In some embodiments, the coating or matrix materials are selected such that the pharmaceutical formulations can survive exposure to gastric acid and release the active ingredient in the intestines after oral administration.

Diluents can increase the bulk of a solid dosage formulation so that a practical size is provided for compression of tablets or formation of beads, granules, or particles. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, powdered sugar, and combinations thereof.

Binders are used to impart cohesive qualities to a solid dosage formulation, and thus ensure that a tablet, bead, granule, or particle remains intact after the formation of the solid dosage formulation. Suitable binders include, but are not limited to, starch, pregelatinized starch, gelatin, sugars (such as sucrose, glucose, dextrose, lactose, and sorbitol), polyethylene glycol, waxes, natural and synthetic gums (such as acacia, tragacanth, and sodium alginate), cellulose (such as hydroxypropylmethylcellulose, hydroxypropylcellulose, and ethylcellulose), veegum, and synthetic polymers (such as acrylic acid copolymers, methacrylic acid copolymers, methyl methacrylate copolymers, aminoalkyl methacrylate copolymers, polyacrylic acid, polymethacrylic acid, and polyvinylpyrrolidone), and combinations thereof.

Lubricants are used to facilitate tablet manufacture. Suitable lubricants include, but are not limited to, magnesium stearate, calcium stearate, stearic acid, glycerol behenate, polyethylene glycol, talc, and mineral oil.

Disintegrants are used to facilitate disintegration or “breakup” of a solid dosage formulation after administration. Suitable disintegrants include, but are not limited to, starch, sodium starch glycolate, sodium carboxymethyl starch, sodium carboxymethylcellulose, hydroxypropyl cellulose, pregelatinized starch, clays, cellulose, gums, and cross-linked polymers, such as cross-linked polyvinylpyrrolidone (e.g., POLYPLASDONE® XL).

Plasticizers are normally present to produce or promote plasticity and flexibility and to reduce brittleness. Examples of plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides.

Stabilizers are used to inhibit or retard decomposition reactions of the active ingredient in the pharmaceutical formulations or stabilize particles in a dispersion. For example, when the decomposition reactions involve an oxidation reaction of the active ingredient in the pharmaceutical formulations, the stabilizer can be an antioxidant or a reducing agent. Stabilizers also include nonionic emulsifiers such as sorbitan esters, polysorbates, and polyvinylpyrrolidone.

Glidants are used to reduce sticking effects during film formation and drying. Exemplary glidants include, but are not limited to, talc, magnesium stearate, and glycerol monostearates.

Preservatives can inhibit the deterioration and/or decomposition of a pharmaceutical formulation. Deterioration or decomposition can be brought about by one or more of microbial growth, fungal growth, and undesirable chemical or physical changes. Suitable preservatives include benzoate salts (e.g., sodium benzoate), ascorbic acid, methyl hydroxybenzoate, ethyl p-hydroxybenzoate, n-propyl p-hydroxybenzoate, n-butyl p-hydroxybenzoate, potassium sorbate, sorbic acid, propionate salts (e.g., sodium propionate), chlorobutanol, benzyl alcohol, and combinations thereof.

Surfactants may be anionic, cationic, amphoteric, or nonionic surface-active agents. Exemplary anionic surfactants include, but are not limited to, those containing a carboxylate, sulfonate, or sulfate ion. Examples of anionic surfactants include sodium, potassium, and ammonium salts of long-chain (e.g., 13-21) alkyl sulfonates (such as sodium lauryl sulfate), alkylaryl sulfonates (such as sodium dodecylbenzene sulfonate), and dialkyl sulfosuccinates (such as sodium bis-(2-ethylthioxyl)-sulfosuccinate). Examples of cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, poloxamers (such as poloxamer 401), stearoyl monoisopropanolamide, and polyoxyethylene hydrogenated tallow amide. Examples of amphoteric surfactants include, but are not limited to, sodium N-dodecyl-β-alanine, sodium N-lauryl-β-iminodipropionate, myristoamphoacetate, lauryl betaine, and lauryl sulfobetaine.

Pharmaceutical formulations in the liquid dosage forms typically contain a solvent or dispersion medium such as water, aqueous solution (e.g., buffer, saline, buffered saline), ethanol, polyol (such as glycerol, propylene glycol, and polyethylene glycol), oil (such as vegetable oil, e.g., peanut oil, corn oil, sesame oil), and combinations thereof. In some embodiments, the pharmaceutical formulations in the liquid dosage forms are aqueous formulations. Suitable solvents or dispersion mediums for aqueous formulations include, but are not limited to, water, buffers (such as acidic buffers), salines (such as normal saline), buffered salines (such as PBS), and Ringer's solution.

In some embodiments, the pharmaceutical formulations contain β-cyclodextrin or derivatives thereof. For example, the pharmaceutical formulations contain sulfobutyl ether β-cyclodextrin. For example, the pharmaceutical formulations contain hydroxypropyl-β-cyclodextrin. Such pharmaceutical formulations can be in a solid dosage form or a liquid dosage form.

### C. Pharmaceutical Acceptable Carriers

In some embodiments, the pharmaceutical formulations are prepared using a pharmaceutically acceptable carrier, which encapsulates, embeds, entraps, dissolves, disperses, absorbs, and/or binds to a compound or composition disclosed herein. The pharmaceutical acceptable carrier is composed of materials that are considered safe and can be administered to a subject without causing undesirable biological side effects or unwanted interactions. Preferably, the pharmaceutically acceptable carrier does not interfere with the effectiveness of the compound or composition in performing its function. The pharmaceutically acceptable carrier can be formed of biodegradable materials, non-biodegradable materials, or combinations thereof. One or more of the pharmaceutical acceptable excipients described above may be present in the pharmaceutical acceptable carrier.

In some embodiments, the pharmaceutical acceptable carrier is a controlled-release carrier, such as delayed-release carriers, sustained-release (extended-release) carriers, and pulsatile-release carriers.

In some embodiments, the pharmaceutical acceptable carrier is pH-sensitive or pH-responsive. In some forms, the pharmaceutical acceptable carrier can decompose or degrade in a certain pH range. In some forms, the pharmaceutical acceptable carrier can experience a structural change when experiencing a change in the pH.

Exemplary pharmaceutical acceptable carriers include, but are not limited to: nanoparticles, microparticles, and combinations thereof, liposomes; hydrogels; polymer matrices; and solvent systems.

In some embodiments, the pharmaceutical acceptable carrier is nanoparticles, microparticles, or a combination thereof. In some embodiments, the compound or composition is embedded in the matrix formed by the materials of the nanoparticles, microparticles, or combination thereof.

The nanoparticles, microparticles, or combination thereof can be biodegradable, and optionally are capable of biodegrading at a controlled rate for delivery of the compound or composition. The nanoparticles, microparticles, or combination thereof can be made of a variety of materials. Both inorganic and organic materials can be used. Both polymeric and non-polymeric materials can be used.

For example, the nanoparticles, microparticles, or combination thereof are formed of one or more biocompatible polymers. In some forms, the biocompatible polymers are biodegradable. In some forms, the biocompatible polymers are non-biodegradable. In some forms, the nanoparticles, microparticles, or combination thereof are formed of a mixture of biodegradable and non-biodegradable polymers. The polymers used to form the nanoparticles, microparticles, or combination thereof may be tailored to optimize different characteristics of the nanoparticles, microparticles, or combination thereof, including: (i) interactions between the active ingredient and the polymer to provide stabilization of the active ingredient and retention of activity upon delivery; (ii) rate of polymer degradation and, thereby, rate of release; (iii) surface characteristics and targeting capabilities; and (iv) particle porosity.

Exemplary polymers include, but are not limited to, polymers prepared from lactones (such as poly(caprolactone) (PCL)), polyhydroxy acids and copolymers thereof (such as poly(lactic acid) (PLA), poly(glycolic acid) (PGA), and poly(lactic acid-co-glycolic acid) (PLGA)), polyalkyl cyanoacralate, polyurethanes, polyamino acids (such as poly-L-lysine (PLL), poly(valeric acid), and poly-L-glutamic acid), hydroxypropyl methacrylate (HPMA), polyanhydrides, polyorthoesters, poly(ester amides), polyamides, poly(ester ethers), polycarbonates, ethylene vinyl acetate polymer (EVA), polyvinyl alcohols (PVA), polyvinyl ethers, polyvinyl esters (such as poly(vinyl acetate)), polyvinyl halides (such as poly(vinyl chloride) (PVC)), polyvinylpyrrolidone, polysiloxanes, polystyrene (PS), celluloses and derivatized celluloses (such as alkyl celluloses, hydroxyalkyl celluloses, cellulose ethers, cellulose esters, nitro celluloses, hydroxypropylcellulose, and carboxymethylcellulose), polymers of acrylic acids (such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate)), polydioxanone, polyhydroxyalkanoates, polypropylene fumarate, polyoxymethylene, poloxamers, poly(butyric acid), polyphosphazenes, polysaccharides, polypeptides, and blends thereof.

In some embodiments, the one or more biocompatible polymers forming the nanoparticles, microparticles, or combination thereof include an FDA-approved biodegradable polymer such as polyhydroxy acids (e.g., PLA, PGA, and PLGA), polyanhydrides, and polyhydroxyalkanoate (e.g., poly(3-butyrate) and poly(4-butyrate)).

Materials other than polymers may be used to form the nanoparticles, microparticles, or combination thereof. Suitable materials include surfactants. The use of surfactants in the nanoparticles, microparticles, or combination thereof may improve surface properties by, for example, reducing particle-particle interactions, and render the surface of the particles less adhesive. Both naturally occurring surfactants and synthetic surfactants can be incorporated into the nanoparticles, microparticles, or combination thereof. Exemplary surfactants include, but are not limited to, phosphoglycerides such as phosphatidylcholines (e.g., L-ax-phosphatidylcholine dipalmitoyl), diphosphatidyl glycerol, hexadecanol, fatty alcohols, polyoxyethylene-9-lauryl ether, fatty acids such as palmitic acid and oleic acid, sorbitan trioleate, glycocholate, surfactin, poloxomers, sorbitan fatty acid esters such as sorbitan trioleate, tyloxapol, and phospholipids.

The nanoparticles, microparticles, or combination thereof may contain a plurality of layers. The layers can have similar or different release kinetic profiles for the active ingredient. For example, the nanoparticles, microparticles, or combination thereof can have a controlled-release core surrounded by one or more additional layers. The one or more additional layers can include an instant-release layer, preferably on the surface of the nanoparticles, microparticles, or combination thereof. The instant-release layer can provide a bolus of the active ingredient shortly after administration.

The composition and structure of the nanoparticles, microparticles, or combination thereof can be selected such that the nanoparticles, microparticles, or combination thereof are pH-sensitive or pH-responsive. In some embodiments, the nanoparticles, microparticles, or combination thereof are formed of one or more pH-sensitive or pH-responsive polymers such as the enteric polymers commercially available under the tradename EUDRAGIT®, as described above. Depending on the particle materials, the decomposition/degradation or structural change of the nanoparticles, microparticles, or combination thereof may occur at different locations of the gastrointestinal tract.

In some embodiments, the particle materials are selected such that the nanoparticles, microparticles, or combination thereof can survive exposure to gastric acid and release the active ingredient in the intestines after oral administration.

### D. Controlled Release

In some embodiments, the pharmaceutical formulations can be controlled-release formulations. Examples of controlled-release formulations include extended-release formulations, delayed-release formulations, and pulsatile-release formulations.

### 1. Extended Release

In some embodiments, the extended-release formulations are prepared as diffusion or osmotic systems, for example, as described in “Remington—The science and practice of pharmacy” (20th Ed., Lippincott Williams & Wilkins, 2000).

A diffusion system is typically in the form of a matrix, generally prepared by combining the active ingredient with a slowly dissolving, pharmaceutically acceptable carrier, optionally in a tablet form. Suitable materials used in the preparation of the matrix include plastics, hydrophilic polymers, and fatty compounds. Suitable plastics include, but are not limited to, acrylic polymer, methyl acrylate-methyl methacrylate copolymer, polyvinyl chloride, and polyethylene. Suitable hydrophilic polymers include, but are not limited to, cellulosic polymers such as methyl ethyl cellulose, hydroxyalkylcelluloses (such as hydroxypropylcellulose and hydroxypropylmethylcellulose), sodium carboxymethylcellulose, CARBOPOL® 934, polyethylene oxides, and combinations thereof. Suitable fatty compounds include, but are not limited to, various waxes such as carnauba wax and glyceryl tristearate, wax-type substances such as hydrogenated castor oil and hydrogenated vegetable oil, and combinations thereof.

In some embodiments, the plastic is a pharmaceutically acceptable acrylic polymer. In some embodiments, the pharmaceutically acceptable acrylic polymer is chosen from acrylic acid and methacrylic acid copolymers, methyl methacrylate copolymers, ethoxyethyl methacrylate copolymers, cyanoethyl methacrylate copolymers, aminoalkyl methacrylate copolymers, poly(acrylic acid), poly(methacrylic acid), methacrylic acid alkylamine copolymers, poly(methyl methacrylate), poly(methacrylic acid), polymethacrylate, polyacrylamide, poly(methacrylic acid anhydride), and glycidyl methacrylate copolymers.

In some embodiments, the pharmaceutically acceptable acrylic polymer can be an ammonio methacrylate copolymer. Ammonio methacrylate copolymers are well known in the art and are described as fully polymerized copolymers of acrylic and methacrylic acid esters with a low content of quaternary ammonium groups.

In some embodiments, the pharmaceutically acceptable acrylic polymer is an acrylic resin lacquer such as those commercially available under the tradename EUDRAGIT®. In some embodiments, the pharmaceutically acceptable acrylic polymer contains a mixture of two acrylic resin lacquers, EUDRAGIT® RL (such as EUDRAGIT® RL30D) and EUDRAGIT® RS (such as EUDRAGIT® RS30D). EUDRAGIT® RL30D and EUDRAGIT® RS30D are copolymers of acrylic and methacrylic acid esters with a low content of quaternary ammonium groups, the molar ratio of ammonium groups to the remaining neutral methacrylic esters being 1:20 in EUDRAGIT® RL30D and 1:40 in EUDRAGIT® RS30D. The code designations RL (high permeability) and RS (low permeability) refer to the permeability properties of these polymers. EUDRAGIT® RL/RS mixtures are insoluble in water and in digestive fluids. However, multi-particulate systems formed to include the same are swellable and permeable in aqueous solutions and digestive fluids. The EUDRAGIT® RL/RS mixtures may be prepared in any desired ratio in order to ultimately obtain a sustained-release formulation having a desirable release profile. Suitable sustained-release, multi-particulate systems may be obtained, for instance, from 90% EUDRAGIT® RL+10% EUDRAGIT® RS, to 50% EUDRAGIT® RL+50% EUDRAGIT® RS, and to 10% EUDRAGIT® RL+90% EUDRAGIT® RS. In some embodiments, the pharmaceutically acceptable acrylic polymer can also be or include other acrylic resin lacquers, such as EUDRAGIT®5-100, EUDRAGIT® L-100, and mixtures thereof.

Matrices with different release mechanisms or profiles can be combined in a final dosage form containing single or multiple units. Examples of multiple units include, but are not limited to, multilayer tablets and capsules containing beads, granules, and/or particles of the active ingredient. An immediate release portion can be added to the extended-release system by means of either applying an immediate release layer on top of the extended-release core using a coating or compression process or in a multiple unit system such as a capsule containing both extended- and immediate-release beads.

Extended-release tablets containing one or more of the hydrophilic polymers can be prepared by techniques commonly known in the art such as direct compression, wet granulation, and dry granulation.

Extended-release tablets containing one or more of the fatty compounds can be prepared using methods known in the art such as direct blend methods, congealing methods, and aqueous dispersion methods. In the congealing methods, the active ingredient is mixed with the fatty compound(s) and congealed.

Alternatively, the extended-release formulations can be prepared using osmotic systems or by applying a semi-permeable coating to a solid dosage form. In the latter case, the desired release profile can be achieved by combining low permeable and high permeable coating materials in suitable proportions.

### 2. Delayed Release

Delayed-release formulations can be prepared by coating a solid dosage form with a coating. In some embodiments, the coating is insoluble and impermeable in the acidic environment of the stomach, and becomes soluble or permeable in the less acidic environment of the intestines and/or the lower GI tract. In some embodiments, the solid dosage form is a tablet for incorporation into a capsule, a tablet for use as an inner core in a “coated-core” dosage form, or a plurality of beads, granules, and/or particles containing the active ingredient, for incorporation into either a tablet or capsule.

Suitable coating materials may be bioerodible polymers, gradually hydrolysable polymers, gradually water-dissolvable polymers, and enzymatically degradable polymers. In some embodiments, the coating material is or contains enteric polymers. Combinations of different coating materials may also be used. Multilayer coatings using different coating materials may also be applied.

The coating may also contain one or more additives, such as plasticizers as described above (optionally representing about 10 wt % to 50 wt % relative to the dry weight of the coating), colorants as described above, stabilizers as described above, glidants as described above, etc.

### 3. Pulsatile Release

Pulsatile-release formulations release a plurality of doses of the active ingredient at spaced-apart time intervals. Generally, upon administration, such as oral administration, of the pulsatile-release formulations, release of the initial dose is substantially immediate, e.g., the first release “pulse” occurs within about three hours, two hours, or one hour of administration. This initial pulse may be followed by a first time-interval (lag time) during which very little or no active ingredient is released from the formulations, after which a second dose may be released. Similarly, a second lag time (nearly release-free interval) between the second and third release pulses may be designed. The duration of the lag times will vary depending on the formulation design, especially on the length of the dosing interval, e.g., a twice daily dosing profile, a three-time daily dosing profile, etc.

For pulsatile-release formulations providing a twice daily dosage profile, they deliver two release pulses of the active ingredient. In some embodiments, the one nearly release-free interval between the first and second release pulses may have a duration of between 3 hours and 14 hours.

For pulsatile-release formulations providing a three daily dosage profile, they deliver three release pulses of the active ingredient. In some embodiments, the two nearly release-free interval between two adjacent pulses may have a duration of between 2 hours and 8 hours.

In some embodiments, the pulsatile-release formulations contain a plurality of pharmaceutically acceptable carriers with different release kinetics.

In some embodiments, the pulsatile-release formulations contain a pharmaceutically acceptable carrier with a plurality of layers loaded with the active ingredient. In some embodiments, the layers may have different release kinetics. In some embodiments, the layers may be separated by a delayed-release coating. For example, the pulsatile-release formulations may have a first layer loaded with the active ingredient on the surface for the first release pulse and a second layer, e.g., a core loaded with the active ingredient, for the second release pulse; the second layer may be surrounded by a delayed-release coating, which creates a lag time between the two release pulses.

In some embodiments, the pulsatile-release profile is achieved with formulations that are closed and optionally sealed capsules housing at least two “dosage units” wherein each dosage unit within the capsules provides a different release profile. In some embodiments, at least one of the dosage units is a delayed-release dosage unit. Control of the delayed-release dosage unit(s) may be accomplished by a controlled-release polymer coating on the dosage unit(s) or by incorporation of the active ingredient in a controlled-release polymer matrix. In some embodiments, each dosage unit may comprise a compressed or molded tablet, wherein each tablet within the capsule provides a different release profile.

### E. Exemplary Formulations for Different Routes of Administration

A subject suffering from a condition, disorder, or disease as described herein, can be treated by either targeted or systemic administration, via oral, inhalation, topical, trans- or sub-mucosal, subcutaneous, intramuscular, intravenous, or transdermal administration of a pharmaceutical formulation containing a compound or composition described herein. In some embodiments, the pharmaceutical formulation is suitable for oral administration. In some embodiments, the pharmaceutical formulation is suitable for subcutaneous, intravenous, or intramuscular administration. In some embodiments, the pharmaceutical formulation is suitable for inhalation or intranasal administration. In some embodiments, the pharmaceutical formulation is suitable for transdermal or topical administration.

In some embodiments, the pharmaceutical formulation is an oral pharmaceutical formulation. In some embodiments, the active ingredient may be incorporated with one or more pharmaceutically acceptable excipients as described above and used in the form of tablets, pills, caplets, or capsules. For example, the corresponding oral pharmaceutical formulation may contain one or more of the following pharmaceutically acceptable excipients or those of a similar nature: a binder as described above, a disintegrant as described above, a lubricant as described above, a glidant as described above, a sweetening agent (such as sucrose and saccharin), and a flavoring agent (such as methyl salicylate and fruit flavorings). In some embodiments, when the oral pharmaceutical formulation is in the form of capsules, it may contain, in addition to the material(s) listed above, a liquid carrier (such as a fatty oil). In some embodiments, when the oral pharmaceutical formulation is in the form of capsules, each capsule may contain a plurality of beads, granules, and/or particles of the active ingredient. In some embodiments, the oral pharmaceutical formulation may contain one or more other materials which modify the physical form or one or more pharmaceutical properties of the dosage unit, for example, coatings of polysaccharides, shellac, or enteric polymers as described in previous sections.

In some embodiments, the oral pharmaceutical formulation can be in the form of an elixir, suspension, syrup, wafer, chewing gum or the like. A syrup may contain, in addition to the active ingredient, one or more sweetening agents (such as sucrose and saccharine), one or more flavoring agents, one or more preservatives, and/or one or more dyes or colorings.

In some embodiments, the pharmaceutical formulation is a subcutaneous, intramuscular, or intravenous pharmaceutical formulation. In some embodiments, the subcutaneous, intramuscular, or intravenous pharmaceutical formulation can be enclosed in an ampoule, syringe, or a single or multiple dose vial made of glass or plastic. In some embodiments, the subcutaneous, intramuscular, or intravenous pharmaceutical formulation contains a liquid pharmaceutically acceptable carrier for the active ingredient. Suitable liquid pharmaceutically acceptable carriers include, but are not limited to, water, buffer, saline, buffered saline (such as PBS), and combinations thereof.

In some embodiments, the pharmaceutical formulation is a topical pharmaceutical formulation. Suitable forms of the topical pharmaceutical formulation include lotions, suspensions, ointments, creams, gels, tinctures, sprays, powders, pastes, slow-release transdermal patches, and suppositories for application to rectal, vaginal, nasal, or oral mucosa. In some embodiments, thickening agents, emollients (such as mineral oil, lanolin and its derivatives, and squalene), humectants (such as sorbitol), and/or stabilizers can be used to prepare the topical pharmaceutical formulations. Examples of thickening agents include petrolatum, beeswax, xanthan gum, and polyethylene.

In some embodiments, the pharmaceutical formulation is an intranasal pharmaceutical formulation. In some embodiments, the intranasal pharmaceutical formulation is in the form of an aqueous suspension, which can be optionally placed in a pump spray bottle. Other than water, the aqueous suspension may contain one or more pharmaceutically acceptable excipients, such as suspending agents (e.g., microcrystalline cellulose, sodium carboxymethylcellulose, hydroxypropyl-methyl cellulose), humectants (e.g., glycerol, propylene glycol), acids, bases, and/or pH-buffering agents for adjusting the pH (e.g., citric acid, sodium citrate, phosphoric acid, sodium phosphate, and combinations thereof), surfactants (e.g., polysorbate 80), and preservatives (e.g., benzalkonium chloride, phenylethyl alcohol, potassium sorbate).

In some embodiments, the pharmaceutical formulation is an inhalation pharmaceutical formulation. In some embodiments, the inhalation pharmaceutical formulation may be in the form of an aerosol suspension, a dry powder, or a liquid suspension. The inhalation pharmaceutical formulation may be prepared for delivery as a nasal spray or an inhaler, such as a metered dose inhaler (MDI). In some embodiments, MDIs can deliver aerosolized particles suspended in chlorofluorocarbon propellants such as CFC-11 and CFC-12, or non-chlorofluorocarbons or alternate propellants such as fluorocarbons (e.g., HFC-134A, HFC-227), with or without surfactants or suitable bridging agents. Dry-powder inhalers can also be used, either breath activated or delivered by pressure.

In some embodiments, the active ingredient is prepared with a pharmaceutically acceptable carrier that will protect it against rapid degradation or elimination from the body of the subject after administration, such as the controlled-release formulations described in previous sections.

### V. Methods of Using

Disclosed are methods of treating a condition, disorder, or disease in a subject in need thereof. The methods include administering an effective amount of a compound, composition, or pharmaceutical formulation disclosed herein to the subject.

The compound, composition, or pharmaceutical formulation can be administered in a variety of manners, depending on whether local or systemic administration is desired. In some embodiments, the compound, composition, or pharmaceutical formulation is directly administered to a specific bodily location of the subject, e.g., topical administration and intranasal administration. In some embodiments, the compound, composition, or pharmaceutical formulation is administered in a systemic manner, such as enteral administration (e.g., oral administration) and parenteral administration (e.g., injection, infusion, and implantation). Exemplary administration routes include oral administration, intravenous administration such as intravenous injection or infusion, intramuscular administration such as intramuscular injection, intranasal administration, and topical administration. In some embodiments, the compound, composition, or pharmaceutical formulation is administered orally. In some embodiments, the compound, composition, or pharmaceutical formulation is administered intravenously. In some embodiments, the compound, composition, or pharmaceutical formulation is administered intramuscularly.

In some embodiments, the subject is a human. In some embodiments, the subject is an adult patient. In some embodiments, the subject is a pediatric patient. In some embodiments, the subject is a non-human animal, such as domestic pets, livestock and farm animals, and zoo animals. In some embodiments, the non-human animal may be a non-human primate.

### A. Indications

The utility of the compounds, compositions, and pharmaceutical formulations of this disclosure may be applied to conditions, disorders, and diseases that can lead to neurological damage, neuronal loss, cerebral edema, and/or neuroinflammation.

Exemplary conditions, disorders, and diseases that can be treated by the disclosed compounds, compositions, and formulations include, but are not limited to, stroke, subarachnoid hemorrhage, cerebral ischemia, cerebral vasospasm, hypoxia, CNS injury (such as head injury and spinal cord injury), concussion, traumatic brain injury, depression, postpartum depression, epilepsy, seizure disorder, essential tremor, fragile X syndrome, and neurodegenerative disease.

In some embodiments, the condition, disorder, or disease is chosen from stroke, subarachnoid hemorrhage, traumatic brain injury, concussion, dementia, Alzheimer's diseases, epilepsy, seizure disorder, depression, and postpartum depression.

In some embodiments, the condition, disorder, or disease is stroke. In some embodiments, the compound, composition, or pharmaceutical formulation is used to treat or prevent stroke-associated damages. In some embodiments, the compound, composition, or pharmaceutical formulation is administered under emergency care for stroke, for maintenance treatment of stroke, and/or for rehabilitation of stroke.

In some embodiments, the condition, disorder, or disease is subarachnoid hemorrhage (SAH). In some embodiments, the compound, composition, or pharmaceutical formulation is used to treat or prevent SAH-associated damages. In some embodiments, the compound, composition or pharmaceutical formulation is administered under emergency care for a SAH, for maintenance treatment of SAH, and/or for rehabilitation of SAH.

SAH refers to an abnormal condition in which blood collects beneath the arachnoid mater, a membrane that covers the brain. This area, called the subarachnoid space, normally contains cerebrospinal fluid. The accumulation of blood in the subarachnoid space, and the vasospasm of the vessels which results from it, can lead to stroke, seizures, and other complications. SAH can be spontaneous or caused by a head injury. The compound, composition, or pharmaceutical formulation can be used to treat a subject experiencing SAH. For example, the compound, composition, or pharmaceutical formulation can be used to prevent or limit one or more of the toxic effects of SAH, including, for example, stroke and ischemia that can result from SAH. Alternatively, the compound, composition, or pharmaceutical formulation can be used to treat a subject with traumatic subarachnoid hemorrhage caused by a head injury.

In some embodiments, the condition, disorder, or disease is cerebral ischemia. In some embodiments, the compound, composition, or pharmaceutical formulation is used to treat or prevent cerebral ischemia-associated damages. In some embodiments, the compound, composition, or pharmaceutical formulation is administered under emergency care for a cerebral ischemia event, for maintenance treatment of cerebral ischemia, and/or for rehabilitation of cerebral ischemia. In some embodiments, the cerebral ischemia is caused by traumatic brain injury, coronary artery bypass graft, carotid angioplasty, or neonatal ischemia following hypothermic circulatory arrest.

In some embodiments, the condition, disorder, or disease is cerebral vasospasm. In some embodiments, the cerebral vasospasm is caused or induced by SAH.

In some embodiments, the condition, disorder, or disease is depression or postpartum depression. In some embodiments, the depression is treatment-resistant depression. In some embodiments, the depression is major depressive disorder. In some embodiments, the depression is bipolar depression.

In some embodiments, the condition, disorder, or disease is a neurodegenerative disease such as dementia and Alzheimer's disease. In some embodiments, the compound, composition, or pharmaceutical formulation is used to reduce one or more symptoms of the neurodegenerative disease. In some embodiments, the compound, composition, or pharmaceutical formulation is used to provide cognitive enhancement to the subject that suffers from the neurodegenerative disease.

In some embodiments, the neurodegenerative disease is Alzheimer's disease. In some embodiments, the neurodegenerative disease is Parkinson's disease. In some embodiments, the neurodegenerative disease is Huntington's disease. In some embodiments, the neurodegenerative disease is dementia. In some embodiments, the dementia is AIDS-induced dementia.

In some embodiments, the condition, disorder, or disease is epilepsy or seizure disorder. In some embodiments, the epilepsy or seizure disorder may be selected from epilepsies that are inadequately controlled by existing medications (i.e., treatment-resistant epilepsy), infantile spasms, and epilepsies or seizure disorders caused by a rare disease or genetic condition (e.g., genetic mutation) that produces epilepsies, seizures, spasms, abnormally hypersynchronous brain activity, and/or other conditions associated with enhanced neuronal synchrony. In some embodiments, the subject may be a pediatric patient suffering from the epilepsy or seizure disorder. In some embodiments, the subject may be an adult patient suffering from the epilepsy or seizure disorder. In some embodiments, the compound, composition, or pharmaceutical formulation is used to reduce the severity and/or intensity of the epilepsy or seizure disorder. In some embodiments, the compound, composition, or pharmaceutical formulation is used to reduce the frequency of the epilepsy or seizure disorder. In some embodiments, the epilepsy is refractory epilepsy, such as Refractory Status Epilepticus. In some embodiments, the epilepsy is CDKL5 Deficiency Disorder. In some embodiments, the epilepsy is Established Status Epilepticus. In some embodiments, the epilepsy is Tuberous Sclerosis Complex. In some embodiments, the epilepsy is Lennox-Gastaut Syndrome. In some embodiments, the epilepsy is Dravet Syndrome. In some embodiments, the epilepsy is Developmental and Epileptic Encephalopathy.

In some embodiments, the condition, disorder, or disease is hypoxia. In some embodiments, the compound, composition, or pharmaceutical formulation is used to treat or prevent hypoxia-associated damages. In some embodiments, the compound, composition, or pharmaceutical formulation is administered under emergency care for a hypoxia event, for maintenance treatment of hypoxia, and/or for rehabilitation of hypoxia. In some embodiments, the hypoxia is induced by respiratory insufficiency, prolonged use of ventilator, or both. In some embodiments, the respiratory insufficiency, prolonged use of ventilator, or both is associated with COVID-19, including hospitalization caused by COVID-19.

In some embodiments, the condition, disorder, or disease is fragile X syndrome.

In some embodiments, the condition, disorder, or disease is essential tremor.

In some embodiments, the condition, disorder, or disease is anxiety, such as Generalized Anxiety Disorder.

In some embodiments, the condition, disorder, or disease is or is induced by Acute GABA Hypofunction.

In some embodiments, the condition, disorder, or disease is cognitive dysfunction or impairment. In some embodiments, cognitive dysfunction or impairment is Huntington's Disease Cognitive Dysfunction. In some embodiments, cognitive dysfunction or impairment is Parkinson's Disease Cognitive Dysfunction. In some embodiments, cognitive dysfunction or impairment is Alzheimer's Disease Cognitive Dysfunction.

The utility of the compounds, compositions, and pharmaceutical formulations of this disclosure may also be applied to multiple sclerosis, arthritis, and cancer.

### B. Dosing and Administration

In some embodiments, the compound, composition, or pharmaceutical formulation is administered for a sufficient time period to alleviate one or more undesired symptoms and/or one or more clinical signs associated with the condition, disorder, or disease being treated. In some embodiments, the compound, composition, or pharmaceutical formulation is administered less than three times daily. In some embodiments, the compound, composition, or pharmaceutical formulation is administered once or twice daily. In some embodiments, the compound, composition, or pharmaceutical formulation is administered once daily. In some embodiments, the compound, composition, or pharmaceutical formulation is administered in a single oral dosage once a day. In some embodiments, the compound, composition, or pharmaceutical formulation is administered in a single intravenous dosage once a day. In some embodiments, the compound, composition, or pharmaceutical formulation is administered in a single intramuscular dosage once a day.

In cases of acute brain injuries, such as stroke, concussion, and traumatic brain injury, the compound, composition, or pharmaceutical formulation may be administered under emergency care via intramuscular injection to minimize the onset of action.

In cases of chronic or non-acute illnesses, such as depression and postpartum depression, the compound, composition, or pharmaceutical formulation may be administered via oral administration or intravenous infusion.

### EXAMPLES

The examples below describe studies to synthesize and evaluate neurosteroids and their prodrugs. Prophetic examples are provided to describe how to synthesize and evaluate deuterated or halogenated analogs of the neurosteroids and prodrugs.

The synthetic methods disclosed herein are compatible with a wide variety of functional groups and starting materials. Thus, a wide variety of compounds can be obtained from the disclosed methods.

Additional synthetic methods and procedures relevant to this disclosure can be found in the following documents, which are incorporated herein by reference. Methods for installing an oxime group at the C20 position of neurosteroids can be found in PCT/US2022/049479 and PCT/US2022/049489. Methods for installing a non-phosphate prodrug moiety disclosed herein can be found in PCT/US2022/049479 and PCT/US2022/049489. Methods for installing a phosphate prodrug moiety disclosed herein can be found in WO2015/023593. Methods for installing deuterium atoms in neurosteroids can be found in WO2021/168106.

General information for synthetic chemistry: All chemicals were purchased from commercial vendors and used without further purification unless stated otherwise. Dichloromethane (DCM), toluene, dimethylformamide (DMF), tetrahydrofuran (THF), ether, N,N-diisopropylethylamine (DIPEA) and triethylamine (TEA) were purchased anhydrous in septum-sealed bottles from Sigma Aldrich. All reactions were conducted using oven- or flame-dried glassware under an inert atmosphere of argon unless noted otherwise. Thin layer chromatography (TLC) was utilized to monitor reaction progress using silica gel 60 F254 aluminum-backed plates. TLC spots were visualized with UV light, KMnO₄, PMA, or ninhydrin stains. Flash chromatography was performed using a Teledyne Isco CombiFlash Rf® system using RediSep® Rf silica gel disposable flash columns (60 Å pore size, 40-60 μm particle size). NMR spectra were acquired using a 400 MHz Varian INOVA or a 600 MHz Bruker Avance III NMR spectrometer. Chemical shifts are reported in δ ppm and referenced using residual solvent peaks (CDCl₃ or TMS). Rotamer signals are denoted with *. High resolution mass spectrometry (HRMS) was performed on a Thermo Exactive Plus Orbitrap Mass Spectrometer using APCI or ESI ionization methods.

### Example 1. Preparation of tert-butyl (S)-2-((methylamino)methyl)pyrrolidine-1-carboxylate (4)

tert-Butyl (S)-2-((methylamino)methyl)pyrrolidine-1-carboxylate (4) was prepared according to Scheme 1.

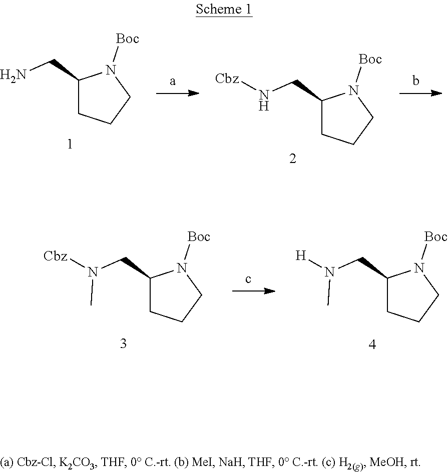 [SMILES: CC(C)(C)OC(=O)N1CCC[C@H]1CN ; CC(C)(C)OC(=O)N1CCC[C@H]1CNC(=O)OCc1ccccc1 ; CN(C[C@@H]1CCCN1C(=O)OC(C)(C)C)C(=O)OCc1ccccc1 ; CNC[C@@H]1CCCN1C(=O)OC(C)(C)C]

### A. Preparation of tert-butyl (S)-2-((((benzyloxy)carbonyl)amino)methyl)pyrrolidine-1-carboxylate (2)

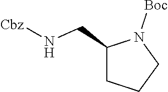 [SMILES: CC(C)(C)OC(=O)N1CCC[C@H]1CNC(=O)OCc1ccccc1]

An oven-dried 100 mL Schlenk tube was charged with a stirrer bar and tert-butyl (S)-2-(aminomethyl)pyrrolidine-1-carboxylate (1) (1500.00 mg, 7.49 mmol) and placed under argon. Anhydrous THF (20 mL) was then added followed by potassium carbonate (2.07 g, 14.98 mmol). The mixture was then cooled to 0° C. in a brine ice bath. Benzyl chloroformate (1.06 mL, 7.49 mmol) was then added dropwise, and the mixture was allowed to warm to room temperature and stirred overnight. Afterwards, the reaction was quenched with 20 mL water and extracted with 50 mL ethyl acetate. The organic layer was separated and washed with saturated ammonium chloride solution and brine. The organic layer was then dried over anhydrous sodium sulfate and concentrated in vacuo to afford an orange oil. Flash chromatography (0-50% ethyl acetate in hexanes) afforded tert-butyl (2S)-2-(benzyloxycarbonylaminomethyl)pyrrolidine-1-carboxylate (2) (2330 mg, 6.9675 mmol, 93% yield) as a colorless oil. The corresponding ¹H NMR spectrum corresponds with the literature (Japanese Patent Application No. JP 2009298710).

### B. Preparation of tert-butyl (2S)-2-((benzyloxycarbonyl(methyl)amino)methyl)pyrrolidine-1-carboxylate (3)

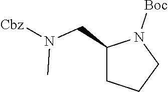 [SMILES: CN(C[C@@H]1CCCN1C(=O)OC(C)(C)C)C(=O)OCc1ccccc1]

An oven-dried 100 mL Schlenk tube was charged with a stirrer bar and tert-butyl (S)-2-((((benzyloxy)carbonyl)amino)methyl)pyrrolidine-1-carboxylate (2) (2.00 g, 5.98 mmol) under argon. Anhydrous THF (25 mL) was then added, and the mixture was cooled to 0° C. in a brine ice bath. Sodium hydride (251.19 mg, 10.47 mmol) was then added to the mixture. After stirring for 30 minutes, methyl iodide (654.44 μL, 10.47 mmol) was added dropwise to mixture at 0° C., and the resulting mixture was allowed to warm to room temperature and stirred overnight (18 h). Afterward, the reaction mixture was quenched by adding a few drops of DI water and then pouring the mixture into saturated ammonium chloride solution (150 mL). The organic layer was then washed with brine and dried over anhydrous sodium sulfate. Concentration in vacuo afforded an orange oil that was subjected to flash chromatography (0-50% ethyl acetate in hexanes) to afford tert-butyl (2S)-2-((benzyloxycarbonyl(methyl)amino)methyl)pyrrolidine-1-carboxylate (3) (1.89 g, 5.4242 mmol, 91% yield) as off-white solid.

### C. Preparation of tert-butyl (S)-2-((methylamino)methyl)pyrrolidine-1-carboxylate (4)

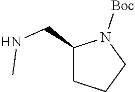

An oven-dried 100 mL round bottom flask was charged with a stirrer bar, tert-butyl (2S)-2-((benzyloxycarbonyl(methyl)amino)methyl)pyrrolidine-1-carboxylate (3) (500.00 mg, 1.43 mmol), palladium on carbon (3.23 mg, 0.0300 mmol), and anhydrous methanol (5 mL) under argon. A T-joint bearing a hydrogen balloon and Schlenk line connection was affixed to the flask. The reaction flask was then briefly evacuated and filled with hydrogen. This process of hydrogen filling was repeated 3 times. The mixture was then allowed to stir at room temperature under hydrogen for 2 h. Afterward, the mixture was filtered over celite, with the celite further washed with methanol. The combined eluants were then concentrated in vacuo to afford a colorless oil that was immediately carried forward to the next reaction.

### Example 2. Preparation of Amine Linkers for Prodrugs of C20-Oxime Neurosteroids (5 and 6)

General Procedure A: To an oven-dried 2-neck round bottom flask, equipped with stirrer bar and molecular sieves (3 Å), was added tert-butyl methyl(2-oxoethyl)carbamate in anhydrous MeOH under an argon atmosphere. The amine nucleophile (2 equiv.) was then added, and the reaction mixture stirred overnight at room temperature. For amine hydrochloride reagents TEA (2 equiv.) was added additionally. The reaction mixture was then treated with palladium on carbon (15 mol %) and stirred under hydrogen atmosphere for 4 hours. Following filtration, the solvent was concentrated in vacuo and purified using flash chromatography.

### A. Preparation of tert-butyl (2-(cyclobutylamino)ethyl)(methyl)carbamate (5)

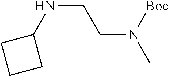 [SMILES: CN(CCNC1CCC1)C(=O)OC(C)(C)C]

Prepared according to General Procedure A using commercially available cyclobutanamine. Rf=0.52 (15% MeOH/DCM). The product was in the form of an oil (870 mg, 3.81 mmol, 66% yield).

### B. Preparation of tert-butyl (2-(isopropylamino)ethyl)(methyl)carbamate (6)

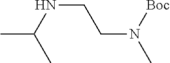

Prepared according to General Procedure A using commercially available propan-2-amine. Rf=0.45 (15% MeOH/DCM). The product was in the form of an oil (507 mg, 2.344 mmol, 52% yield).

### Example 3. Preparation of Ganaxolone and C3-Protected Ganaxolone

Ganaxolone and C3-protected ganaxolone were prepared from dihydroprogesterone according to Scheme 2.

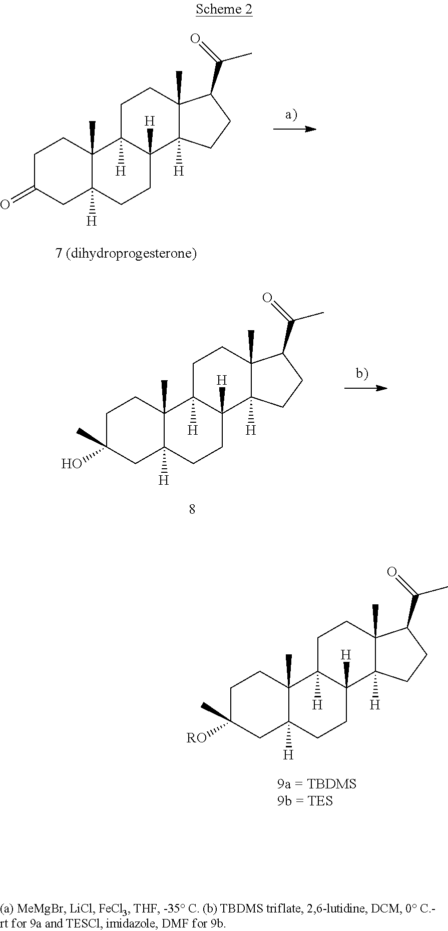

### A. Preparation of 1-((3R,8R,9S,10S,13S,14S,17S)-3-hydroxy-3,10,13-trimethylhexadecahydro-1H-cyclopenta[a]phenanthren-17-yl)ethan-1-one (8)

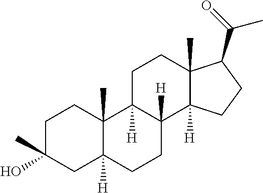

Prepared according to PCT Patent Application Publication No. WO2016/164763, with the product in the form of a white solid (5.1 g, 15.36 mmol, 81% yield). ¹H and ¹³C NMR spectra are consistent with the literature.

### B. Preparation of 1-((3R,8R,9S,10S,13S,14S,17S)-3-((tert-butyldimethylsilyl)oxy)-3,10,13-trimethylhexadecahydro-1H-cyclopenta[a]phenanthren-17-yl)ethan-1-one (9a)

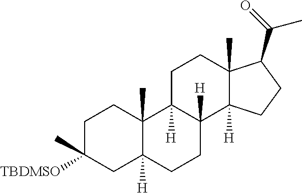

To a flame-dried 2-neck round bottom flask with a stirrer bar was added neurosteroid (8) (4.88 g, 14.6 mmol, 1 equiv.), 2,6-lutidine (5.11 mL, 44 mmol, 3 equiv.) and anhydrous DCM under argon. The mixture was then cooled to 0° C. in a brine ice bath. tert-Butyldimethylsilyl trifluoromethanesulfonate (4.7 mL, 21.9 mmol, 1.5 equiv.) was then added dropwise and the resulting mixture was allowed to stir at 0° C. for 2 h. Afterward, the solution was diluted in DCM and washed with brine. The organic layer was then dried over anhydrous sodium sulfate and concentrated in vacuo. Flash chromatography (10% ethyl acetate:hexanes) afforded the silyl protected products 9a as white solids.

### C. Preparation of 1-((3R,8R,9S,10S,13S,14S,17S)-3,10,13-trimethyl-3-((triethylsilyl)oxy)hexadecahydro-1H-cyclopenta[a]phenanthren-17-yl)ethan-1-one (9b)

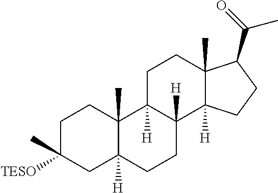

To a flame-dried 2-neck round bottom flask with a stirrer bar was added neurosteroid (8) (4 g, 12.5 mmol, 1 equiv.), imidazole (2.13 g, 31.4 mmol, 2.5 equiv.) and anhydrous DMF (5 mL/mmol 8) under argon. The mixture was then cooled to 0° C. in an ice bath. TESCl (2.5 mL, 15 mmol, 1.2 equiv.) was then added dropwise and the resulting mixture was allowed to stir at RT for 24 h. Afterward, the solution was diluted with ice cold water and extracted it with EtOAc (5 mL/mmol 8) and washed with brine (3×10 mL/mmol 8). The organic layer was then dried over anhydrous sodium sulfate and concentrated in vacuo. Flash chromatography (10% ethyl acetate:hexanes) afforded the silyl protected product 9b as white solid.

### Example 4. Preparation of Ganaxolone C20-Oximes

Ganaxolone C20-oximes were prepared according to Scheme 3. The synthetic procedures are described in General Procedure B.

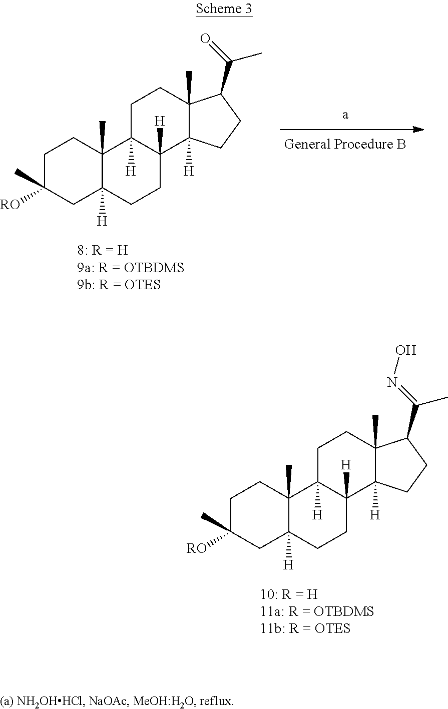

General Procedure B: To a 3-neck R^{B} flask with a stirrer bar, condenser, and addition funnel was added neurosteroid (8 or 9a or 9b) (1 equiv.) and methanol (10 mL/mmol). The mixture was heated to reflux to dissolve the suspended solid (ca. 10 min). A separate solution containing sodium acetate (2.2 equiv.), hydroxylamine hydrochloride (2 equiv.), and water (4 mL/mmol) was then slowly added dropwise via addition funnel (ca. 30 min). After addition, the mixture was allowed to reflux until complete conversion as observed by TLC (3 h). A thick white precipitate formed during the course of the reaction. After completion, the mixture was allowed to cool to room temperature and an additional 5 mL/mmol of water was added, followed by 30 min of stirring. The reaction mixture was then subjected to vacuum filtration, and the resulting white solid was washed with water. Drying of the solid in vacuo afforded the products 10 or 11a-b as white solids.

### A. Preparation of (E)-1-((3R,8R,9S,10S,13S,14S,17S)-3-hydroxy-3,10,13-trimethylhexadecahydro-1H-cyclopenta[a]phenanthren-17-yl)ethan-1-one oxime (10)

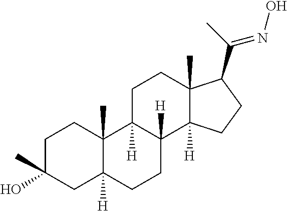

Prepared according to General Procedure B using ganaxolone as the starting neurosteroid. The product was in the form of a white solid (940 mg, 2.71 mmol, 90% yield). ¹H NMR (600 MHz, CDCl3) δ 8.35 (s, 1H), 2.22 (t, J=9.1 Hz, 1H), 2.05 (q, J=10.8 Hz, 1H), 1.87 (s, 3H), 1.86-1.77 (m, 1H), 1.73-1.62 (m, 3H), 1.62-1.41 (m, 5H), 1.41-1.31 (m, 2H), 1.31-1.17 (m, 9H), 1.17-1.04 (m, 3H), 0.93 (qd, J=12.0, 5.4 Hz, 1H), 0.79 (dd, J=11.3, 4.6 Hz, 1H), 0.74 (s, 3H), 0.61 (s, 3H); ¹³C NMR (151 MHz, CDCl₃) δ 159.0, 69.8, 56.9, 56.0, 54.3, 44.1, 41.8, 41.1, 38.9, 35.7, 35.6, 34.9, 34.1, 31.9, 31.6, 28.4, 24.1, 23.1, 21.0, 15.1, 13.4, 11.2. HRMS (ESI+) [M+H]⁺ calc. for C₂₂H₃₈O₂N, 348.28971, observed, 348.2901.

### B. Preparation of (E)-1-((3R,8R,9S,10S,13S,14S,17S)-3-((tert-butyldimethylsilyl)oxy)-3,10,13-trimethylhexadecahydro-1H-cyclopenta[a]phenanthren-17-yl)ethan-1-one oxime (11a)

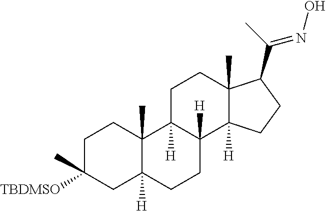

Prepared according to General Procedure B using silyl protected ganaxolone as the starting neurosteroid. The product was in the form of a white solid (1.9 g, 4.12 mmol, 92% yield).

### C. Preparation of (E)-1-((3R,8R,9S,10S,13S,14S,17S)-3,10,13-trimethyl-3-((triethylsilyl)oxy)hexadecahydro-1H-cyclopenta[a]phenanthren-17-yl)ethan-1-one oxime (11b)

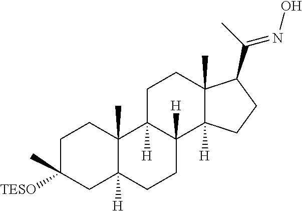

Prepared according to General Procedure B using silyl protected ganaxolone as the starting neurosteroid. The product was in the form of a white solid (86% yield).

### Example 5. Preparation of Fluorine-Substituted Ganaxalone C20-Oxime

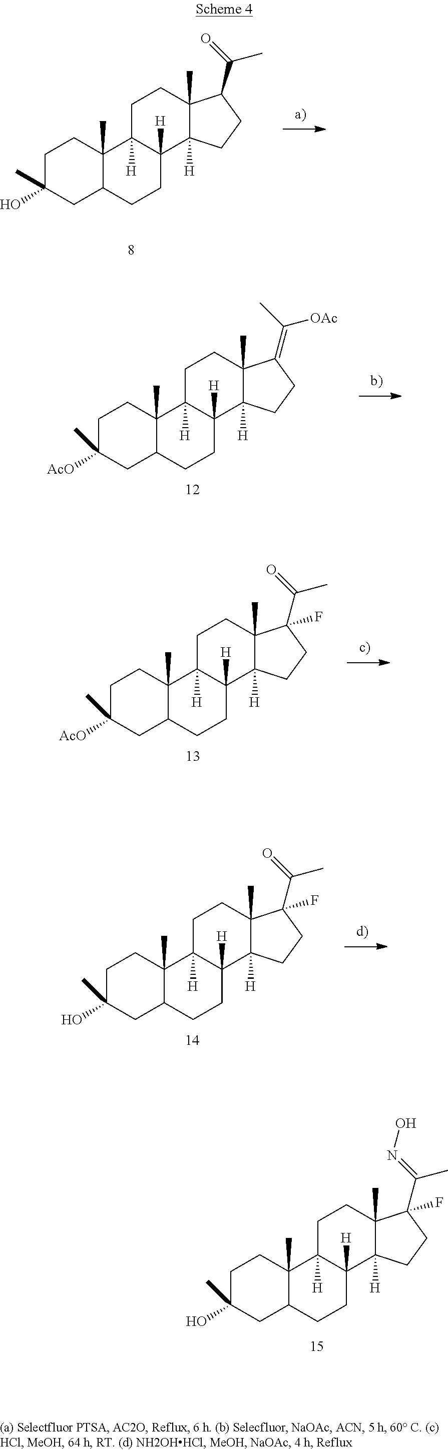

### A. Preparation of (E)-1-((3R,8R,9S,10S,13S,14S)-3-acetoxy-3,10,13-trimethylhexadecahydro-17H-cyclopenta[a]phenanthren-17-ylidene)ethyl acetate (12)

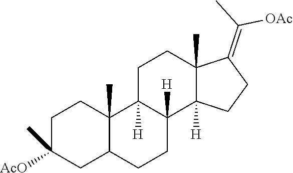

Prepared according to Herzog, et al., J. Am. Chem. Soc., 1960, 82, 14, 3691-3696, with the product in the form of a brown solid (quantitative). The compound was used for the next step without any further purification.

### B. Preparation of (3R,8R,9S,10S,13S,14S,17R)-17-acetyl-17-fluoro-3,10,13-trimethylhexadecahydro-1H-cyclopenta[a]phenanthren-3-yl acetate (13)

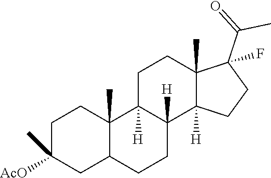

Enol acetate 12 (0.25 mmol), Selectfluor (0.71 mmol, 2.8 mol eq) and sodium acetate (0.05 g) were weighed in a screw-cap vial and dissolved in acetonitrile (3 mL). The reaction was stirred at 60° C. for 2 h, then concentrated under reduced pressure and purified via flash column chromatography (10-50% ethyl acetate in hexanes) to afford fluoride 13 (56%) as a white solid.

### C. Preparation of 1-((3R,8R,9S,10S,13S,14S,17R)-17-fluoro-3-hydroxy-3,10,13-trimethylhexadecahydro-1H-cyclopenta[a]phenanthren-17-yl)ethan-1-one (14)

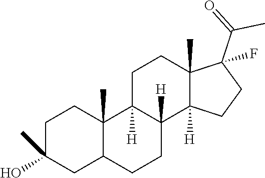

To a solution of acetate 13 (1.26 mmol) dissolved in 5 mL DCM and 45 mL methanol was added 0.2 mL of 12 M HCl, and the reaction was stirred overnight. The reaction mixture was concentrated via reduced pressure, and the crude product was purified via flash column chromatography (20% ethyl acetate in hexanes) to yield 14 (73%) as a white solid.

### D. Preparation of (E)-1-((3R,8R,9S,10S,13S,14S,17R)-17-fluoro-3-hydroxy-3,10,13-trimethylhexadecahydro-1H-cyclopenta[a]phenanthren-17-yl)ethan-1-one oxime

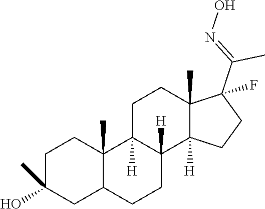

To a 3-neck RB flask with a stirrer bar, condenser, and addition funnel, neurosteroid 14 (1 equiv.) and methanol (10 mL/mmol) were added. The mixture was heated to reflux to dissolve the suspended solid (ca. 10 min). A separate solution containing sodium acetate (2.2 equiv.), hydroxylamine hydrochloride (2 equiv.), and water (4 mL/mmol) was then slowly added dropwise via addition funnel (ca. 30 min). After addition, the mixture was allowed to reflux until complete conversion as observed by TLC (3 h). A thick white precipitate formed during the course of the reaction. After completion, the mixture was allowed to cool to room temperature and an additional (5 mL/mmol 14) of water was added, followed by 30 min of stirring. The reaction mixture was then subjected to vacuum filtration, and the resulting white solid was washed with water (3×3 mL/mmol 14). Drying of the solid in vacuo afforded the product 15 as white solid (quant.). ¹H NMR (600 MHz, CDCl₃) δ 2.81-2.59 (m, 1H), 1.93 (d, J=0.8 Hz, 3H), 1.83-1.63 (m, 5H), 1.62-1.12 (m, 21H), 1.03 (tdd, J=12.8, 11.7, 4.7 Hz, 1H), 0.91-0.80 (m, 1H), 0.78 (d, J=0.6 Hz, 3H), 0.64 (t, J=0.8 Hz, 3H). ¹⁹F NMR (565 MHz, CDCl₃) δ −152.25 (dd, J=39.3, 31.4 Hz).

### Example 6. Preparation of (E)-1-((3R,8R,9S,10S,13S,14S,17S)-3-((tert-butyldimethylsilyl)oxy)-3,10,13-trimethylhexadecahydro-1H-cyclopenta[a]phenanthren-17-yl)ethan-1-one O-((4-nitrophenoxy)carbonyl) oxime

The ganaxolone C20-oxime 4-nitrophenyl carbonate intermediates were prepared according to Scheme 5.

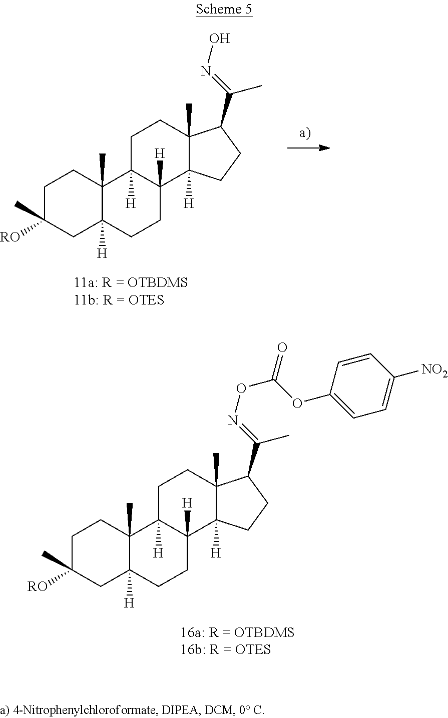

General procedure C for synthesizing nitrophenol esters: To a flame-dried round bottom flask with a stirrer bar was added neurosteroid C20-oxime (11a-b) (2.37 g, 5.14 mmol, 1 equiv.), NEt₃ (1.07 mL, 7.71 mmol, 1.5 equiv.) and anhydrous DCM (5 mL/mmol) under argon. The mixture was then cooled to 0° C. in a brine ice bath. 4-Nitrophenyl chloroformate (1.54 g, 7.71 mmol, 1.5 equiv.) was then added portion-wise and the resulting mixture was allowed to stir at 0° C. for 1.5 h. Afterward, the mixture was concentrated in vacuo, adsorbed onto celite, and subjected to flash chromatography (5% ethyl acetate:DCM) to afford the title compound 16a-b as white solids.

### A. Preparation of (E)-1-((3R,8R,9S,10S,13S,14S,17S)-3-((tert-butyldimethylsilyl)oxy)-3,10,13-trimethylhexadecahydro-1H-cyclopenta[a]phenanthren-17-yl)ethan-1-one O-((4-nitrophenoxy)carbonyl) oxime (16a)

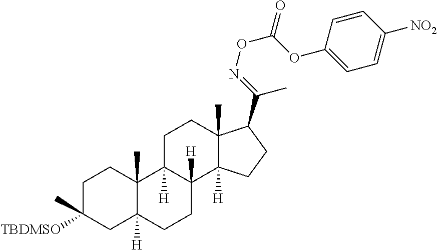

Prepared according to General Procedure C using silyl protected ganaxolone as the starting neurosteroid. The product was in the form of a white solid (2.73 g, 4.36 mmol, 85% yield).

### B. Preparation of (E)-1-((3R,8R,9S,10S,13S,14S,17S)-3,10,13-trimethyl-3-((triethylsilyl)oxy)hexadecahydro-1H-cyclopenta[a]phenanthren-17-yl)ethan-1-one O-((4-nitrophenoxy)carbonyl) oxime (16b)

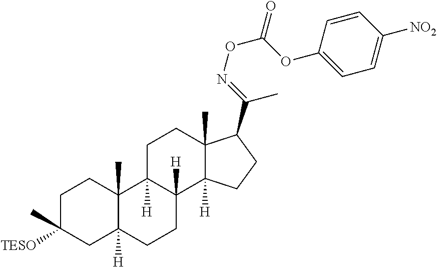

Prepared according to General Procedure C using silyl protected ganaxolone as the starting neurosteroid. The product was in the form of a white solid (78% yield).

### Example 7. Preparation of C20-Oxime Carbamate Prodrugs Using 16a

C20-oxime prodrugs were prepared according to Scheme 6. The synthetic procedures are described in General Procedure D and General Procedure E.

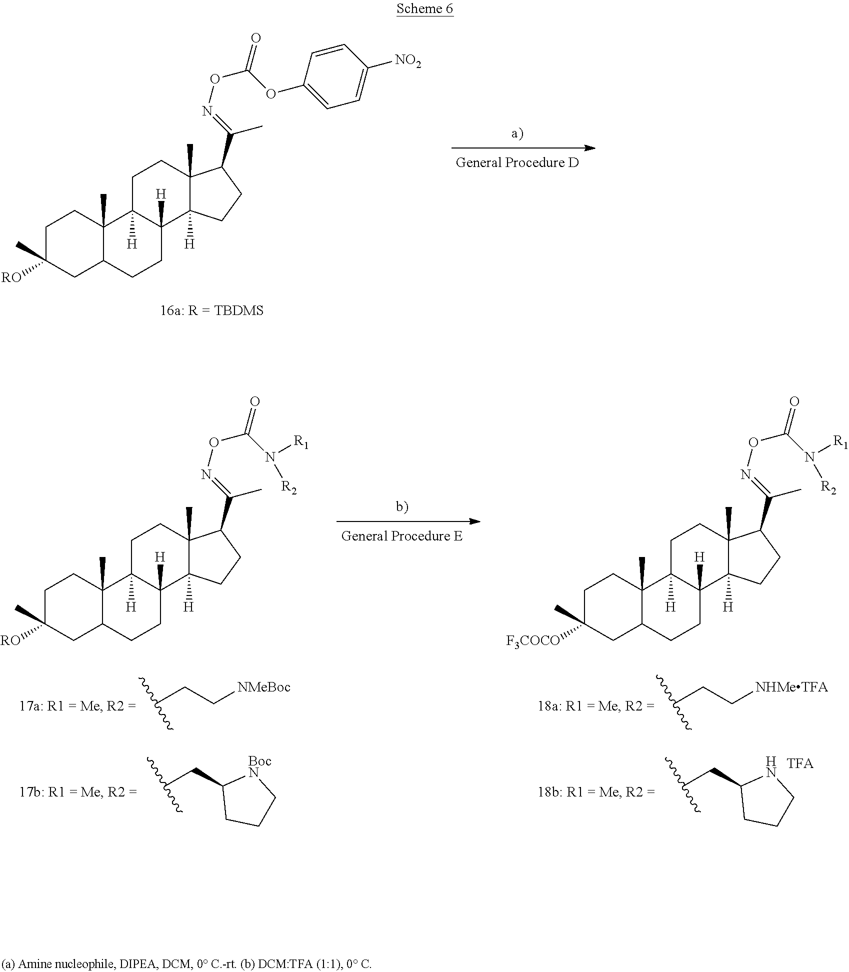

General Procedure D: To a flame-dried 2-neck round bottom flask with a stirrer bar was added compound 16a (1 equiv.), and anhydrous DCM (5 mL/mmol 16a) under argon. The mixture was then cooled to 0° C. in a brine ice bath and DIPEA (2.5 equiv.) was added slowly. The amine nucleophile (1.5 equiv.) was then added, and the resulting mixture was allowed to warm to rt and stirred until consumption of the starting material was observed by TLC (30-60 min). Afterward, the solution was diluted in ethyl acetate (10 mL/mmol 16a) and washed with brine (5×10 mL/mmol 16a brine). The organic layer was then dried over anhydrous sodium sulfate and concentrated in vacuo. Flash chromatography (ethyl acetate:hexanes) afforded the boc protected analogs 17a-b as white solids.

General Procedure E: To a flame-dried vial containing a stirrer bar and Boc-protected analogs 17a-b (1 equiv.) was added anhydrous DCM (3 mL/mmol 17a-b) under argon. The resulting solution was then cooled to 0° C. in a brine ice bath. Trifluoroacetic acid (3 mL/mmol 17a-b) was then added dropwise, and the resulting solution was stirred at 0° C. for 1 h. The resulting solution was then concentrated in vacuo to afford a pink oil. Flash chromatography (methanol:DCM) afforded the prodrugs 18a-b as the trifluoroacetate salt and white solids.

### A. Preparation of tert-butyl (2-((((((E)-1-((3R,8R,9S,10S,13S,14S,17S)-3-((tert-butyldimethylsilyl)oxy)-3,10,13-trimethylhexadecahydro-1H-cyclopenta[a]phenanthren-17-yl)ethylidene)amino)oxy)carbonyl)(methyl)amino)ethyl)(methyl)carbamate (17a)

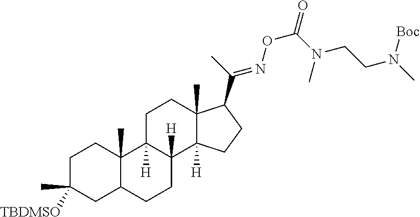

Prepared according to General Procedure D using 16a as the starting 4-nitrophenyl carbonate intermediate and tert-butyl methyl(2-(methylamino)ethyl)carbamate hydrochloride as the amine nucleophile. The product was in the form of a white solid (185 mg, 0.274 mmol, 86%).

### B. Preparation of tert-butyl (2S)-2-(((((((E)-1-((3R,8R,9S,10S,13S,14S,17S)-3-((tert-butyldimethylsilyl)oxy)-3,10,13-trimethylhexadecahydro-1H-cyclopenta[a]phenanthren-17-yl)ethylidene)amino)oxy)carbonyl)(methyl)amino)methyl)pyrrolidine-1-carboxylate (17b)

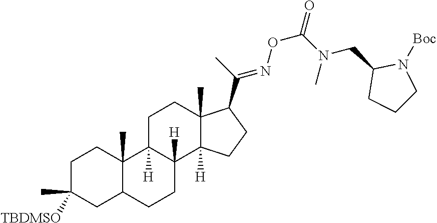

Prepared according to Genera Procedure D using 16a as the starting 4-nitrophenyl carbonate intermediate and tert-butyl (S)-2-((methylamino)methyl)pyrrolidine-1-carboxylate as the amine nucleophile. The product was in the form of a white solid (188 mg, 0.26 mmol, 84%).

### C. Preparation of N-methyl-2-(methyl(((((E)-1-((3R,8R,9S,10S,13S,14S,17S)-3,10,13-trimethyl-3-(2,2,2-trifluoroacetoxy)hexadecahydro-1H-cyclopenta[a]phenanthren-17-yl)ethylidene)amino)oxy)carbonyl)amino)ethan-1-aminium trifluoroacetate (18a)

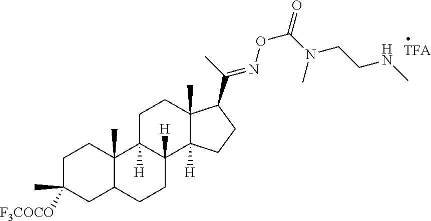

Prepared according to General Procedure E using 17a as the starting Boc-protected analog. The product was in the form of a white solid (58.5 mg, 0.105 mmol, 71%). ¹H NMR (600 MHz, CDCl₃) δ 9.51 (s, 2H), 3.81-3.54 (m, 2H), 3.28 (d, J=13.0 Hz, 2H), 3.00 (s, 3H), 2.75 (s, 3H), 2.35 (t, J=8.9 Hz, 1H), 2.28-2.22 (m, 1H), 2.13 (q, J=10.6 Hz, 1H), 1.99 (dt, J=13.9, 2.5 Hz, 1H), 1.98 (s, 3H), 1.89-1.80 (m, 1H), 1.76-1.68 (m, 4H), 1.64 (d, J=3.0 Hz, 1H), 1.60-1.49 (m, 4H), 1.45-1.08 (m, 10H), 1.02-0.85 (m, 2H), 0.77 (d, J=46.5 Hz, 4H), 0.66 (d, J=2.7 Hz, 3H). ¹³C NMR (151 MHz, CDCl₃) δ 166.5, 162.08 (q, J=34.5 Hz), 156.51, 156.43-155.56 (m), 132.7, 119.72 (d, J=25.3 Hz), 117.52 (d, J=51.4 Hz), 116.11-115.01 (m), 56.99-56.25 (m), 113.5, 90.4, 88.8, 56.99-56.25 (m), 56.25-55.13 (m), 54.49-53.37 (m), 47.6, 46.4, 44.5, 44.5, 44.5, 43.6, 41.8, 40.9, 40.0, 38.71 (d, J=5.1 Hz), 38.6, 38.5, 36.0, 35.77 (d, J=7.2 Hz), 35.5, 35.5, 35.2, 34.6, 34.4, 33.7, 33.4, 32.2, 31.9, 31.74 (d, J=11.1 Hz), 31.6, 28.6, 28.3, 27.8, 25.9, 24.55-23.74 (m), 24.1, 23.2, 23.1, 23.1, 22.6, 21.0, 20.9, 20.9, 16.8, 16.8, 16.7, 13.66-13.32 (m), 11.9, 11.7, 11.6. HRMS (ESI+) [M+H]⁺ calc. for C₂₉H₄₇O₄N₃F₃, 558.35132, observed, 558.3527.

### D. Preparation of (2S)-2-((methyl(((((E)-1-((3R,8R,9S,10S,13S,14S,17S)-3,10,13-trimethyl-3-(2,2,2-trifluoroacetoxy)hexadecahydro-1H-cyclopenta[a]phenanthren-17-yl)ethylidene)amino)oxy)carbonyl)amino)methyl)pyrrolidin-1-ium trifluoroacetate (18b)

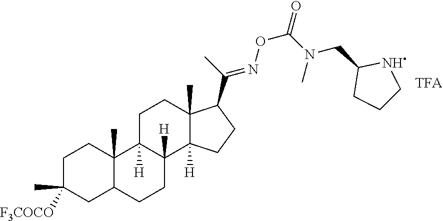

Prepared according to General Procedure E using 17b as the starting Boc-protected analog. The product was in the form of a white solid (57.3 mg, 0.09 mmol, 69%). ¹H NMR (600 MHz, CDCl₃) δ 1H NMR (600 MHz, CDCl₃) δ 11.04 (s, 1H), 8.50 (s, 1H), 3.96 (d, J=11.9 Hz, 2H), 3.45-3.26 (m, 3H), 3.04 (s, 3H), 2.36 (td, J=9.3, 5.1 Hz, 1H), 2.30-2.22 (m, 1H), 2.19-2.08 (m, 3H), 2.08-2.01 (m, 1H), 2.01-1.88 (m, 4H), 1.87-1.77 (m, 2H), 1.77-1.64 (m, 4H), 1.63 (d, J=3.1 Hz, 1H), 1.57 (s, 3H), 1.56-1.49 (m, 2H), 1.42-1.06 (m, 10H), 0.94 (dtd, J=24.3, 12.3, 3.6 Hz, 1H), 0.87 (s, 1H), 0.80 (s, 2H), 0.76-0.73 (m, 1H), 0.72 (s, 1H), 0.66 (d, J=2.5 Hz, 3H). ¹³C NMR (151 MHz, CDCl₃) δ 167.1, 167.0, 162.10 (q, J=34.6 Hz), 157.1, 156.14 (q, J=41.0 Hz), 132.7, 119.71 (d, J=28.7 Hz), 117.51 (d, J=48.4 Hz), 115.59 (d, J=42.8 Hz), 113.66 (d, J=37.0 Hz), 90.4, 88.9, 59.2, 56.51 (d, J=23.8 Hz), 55.71 (d, J=15.2 Hz), 54.1-53.76 (m), 50.9, 44.57 (d, J=10.1 Hz), 43.6, 41.8, 40.9, 40.0, 38.7, 38.6, 38.59-38.29 (m), 36.0, 35.8, 35.7, 35.59-35.29 (m) 35.2, 34.3, 33.7, 32.2, 32.00-31.13 (m), 28.6, 28.3, 27.8, 27.9, 25.9, 24.18 (d, J=17.1 Hz), 24.1, 23.15 (dd, J=15.5, 8.0 Hz), 22.62, 21.27-20.44 (m), 17.08-15.97 (m), 14.06-12.71 (m), 11.9, 11.7, 11.6. HRMS (ESI+) [M+H]⁺ calc. for C₃₁H₄₉O₄N₃F₃, 584.36697, observed, 584.36849.

### Example 8. Preparation of C20-Oxime Carbamate Prodrugs Using 16b

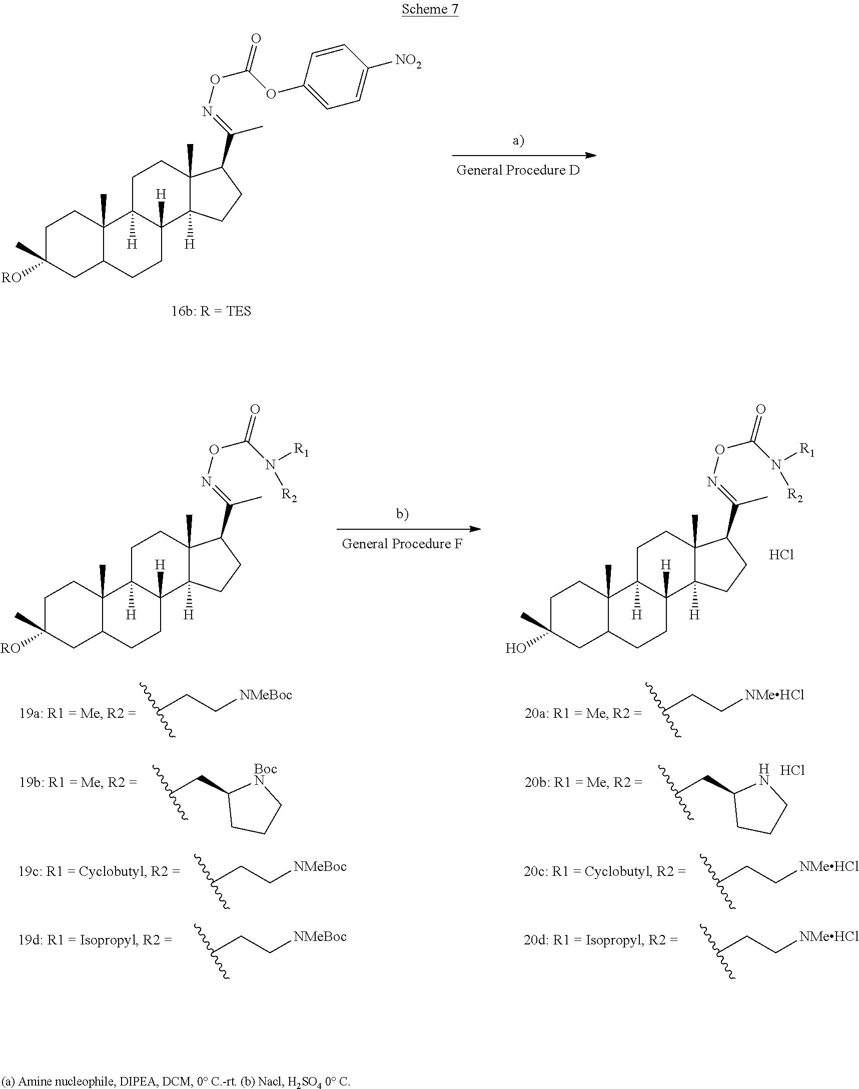

General procedure F: Chamber A of a flame-dried small two-chamber reactor was charged with a stirring bar and sodium chloride (3.0 equiv.). Next, the N-Boc protected substrate is finely ground with a pestle and mortar and the powder (1.0 equiv.) is transferred to (detachable) chamber B. Finally, the reactor was capped and 0.5 mL of concentrated sulfuric acid (H₂SO₄, >95%) was added by injection through the septum in chamber A and instant gas formation was observed. After stirring for 1 to 2 hours at room temperature, one of the caps was carefully removed to release the residual pressure. The reaction was stirred for another few minutes to ensure that all hydrogen chloride gas was extracted out of the fume hood. Next, chamber B was detached to isolate the hydrochloric acid salt by silica gel purification using (20% MeOH/DCM).

### A. Preparation of tert-butyl methyl(2-(methyl(((((E)-1-((3R,8R,9S,10S,13S,14S,17S)-3,10,13-trimethyl-3-((triethylsilyl)oxy)hexadecahydro-1H-cyclopenta[a]phenanthren-17-yl)ethylidene)amino)oxy)carbonyl)amino)ethyl)carbamate (19a)

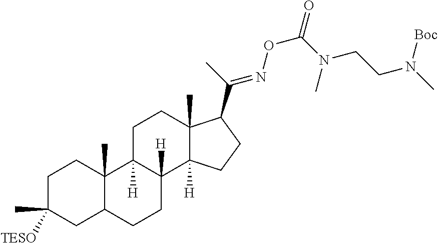

Prepared according to General Procedure D using 16b as the starting 4-nitrophenyl carbonate intermediate and tert-butyl methyl(2-(methylamino)ethyl)carbamate hydrochloride as the amine nucleophile. The product was in the form of a white solid (85%).

### B. Preparation of tert-butyl (2S)-2-((methyl(((((E)-1-((3R,8R,9S,10S,13S,14S,17S)-3,10,13-trimethyl-3-((triethylsilyl)oxy)hexadecahydro-1H-cyclopenta[a]phenanthren-17-yl)ethylidene)amino)oxy)carbonyl)amino)methyl)pyrrolidine-1-carboxylate (19b)

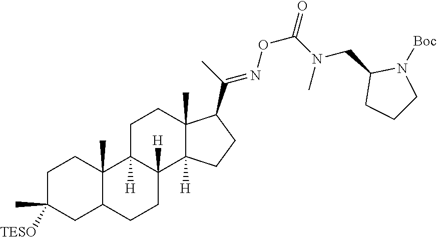

Prepared according to General Procedure D using 16b as the starting 4-nitrophenyl carbonate intermediate and tert-butyl methyl(2-(methylamino)ethyl)carbamate hydrochloride as the amine nucleophile. The product was in the form of a white solid (82%).

### C. Preparation of tert-butyl (2-(cyclobutyl(((((E)-1-((3R,8R,9S,10S,13S,14S,17S)-3,10,13-trimethyl-3-((triethylsilyl)oxy)hexadecahydro-1H-cyclopenta[a]phenanthren-17-yl)ethylidene)amino)oxy)carbonyl)amino)ethyl)(methyl)carbamate (19c)

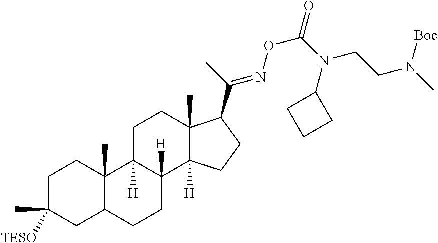

Prepared according to General Procedure D using 16b as the starting 4-nitrophenyl carbonate intermediate and tert-butyl methyl(2-(methylamino)ethyl)carbamate hydrochloride as the amine nucleophile. The product was in the form of a white solid (86%).

### D. Preparation of tert-butyl (2-(isopropyl(((((E)-1-((3R,8R,9S,10S,13S,14S,17S)-3,10,13-trimethyl-3-((triethylsilyl)oxy)hexadecahydro-1H-cyclopenta[a]phenanthren-17-yl)ethylidene)amino)oxy)carbonyl)amino)ethyl)(methyl)carbamate (19d)

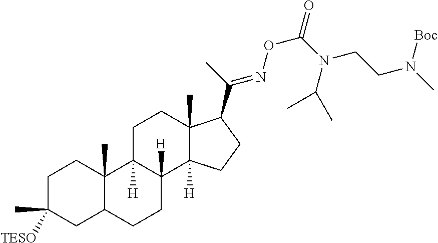

Prepared according to General Procedure D using 16b as the starting 4-nitrophenyl carbonate intermediate and tert-butyl methyl(2-(methylamino)ethyl)carbamate hydrochloride as the amine nucleophile. The product was in the form of a white solid (86%).

### E. Preparation of (E)-1-((3R,8R,9S,10S,13S,14S,17S)-3-hydroxy-3,10,13-trimethylhexadecahydro-1H-cyclopenta[a]phenanthren-17-yl)ethan-1-one O-(methyl(2-(methylamino)ethyl)carbamoyl) oxime hydrochloride (20a)

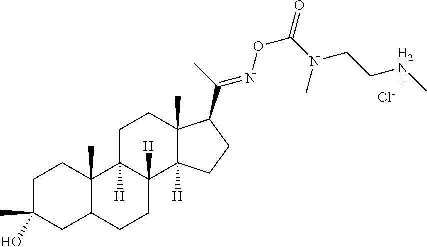

Prepared according to General Procedure F using 19a as the starting Boc-protected analog. The product was in the form of a white solid (49%). ¹H NMR (600 MHz, CDCl₃) δ 9.46 (d, J=98.2 Hz, 2H), 3.83 (t, J=9.3 Hz, 1H), 3.57 (d, J=15.4 Hz, 1H), 3.39-3.17 (m, 2H), 3.00 (s, 3H), 2.74 (s, 3H), 2.36 (t, J=8.9 Hz, 1H), 2.11-2.00 (m, 1H), 1.96 (s, 3H), 1.86-1.65 (m, 8H), 1.63-1.45 (m, 5H), 1.43-1.34 (m, 1H), 1.32-1.09 (m, 12H), 0.97 (tt, J=12.4, 6.4 Hz, 1H), 0.85-0.78 (m, 1H), 0.77 (s, 3H), 0.66 (s, 3H). ¹³C NMR (151 MHz, CDCl₃) δ 167.3, 157.0, 69.8, 56.4, 55.7, 54.1, 47.9, 46.7, 44.6, 41.7, 41.0, 38.5, 35.6, 34.9, 34.6, 34.1, 33.4, 31.8, 31.6, 29.7, 28.3, 24.2, 23.2, 20.9, 16.5, 13.5, 11.2.

### F. Preparation of (E)-1-((3R,8R,9S,10S,13S,14S,17S)-3-hydroxy-3,10,13-trimethylhexadecahydro-1H-cyclopenta[a]phenanthren-17-yl)ethan-1-one O-(methyl(((S)-pyrrolidin-2-yl)methyl)carbamoyl) oxime hydrochloride (20b)

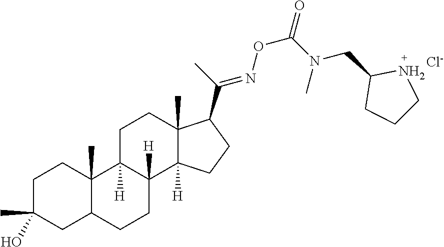

Prepared according to General Procedure F using 19b as the starting Boc-protected analog. The product was in the form of a white solid (38%). 1H NMR (600 MHz, CDCl₃) δ 10.70 (s, 1H), 8.69 (s, 1H), 4.08 (s, 1H), 3.89-3.74 (m, 1H), 3.62-3.43 (m, 2H), 3.37 (d, J=9.0 Hz, 1H), 3.11 (s, 3H), 2.36 (t, J=9.2 Hz, 1H), 2.26-2.09 (m, 4H), 1.98 (s, 3H), 1.93-1.78 (m, 2H), 1.78-1.64 (m, 3H), 1.63-1.44 (m, 6H), 1.38 (t, J=13.4 Hz, 2H), 1.32-1.11 (m, 12H), 0.96 (qd, J=12.5, 4.9 Hz, 1H), 0.81 (d, J=11.2 Hz, 1H), 0.76 (s, 3H), 0.68 (s, 3H). ¹³C NMR (151 MHz, CDCl₃) δ 166.7, 157.3, 69.7, 59.3, 56.9, 56.0, 54.1, 53.4, 51.2, 45.0, 44.5, 44.4, 41.8, 41.1, 38.8, 36.0, 35.7, 35.6, 34.8, 34.0, 31.8, 31.6, 29.6, 28.3, 27.9, 24.2, 23.6, 23.0, 20.9, 17.1, 13.7, 13.6, 11.2.

### G. Preparation of (E)-1-((3R,8R,9S,10S,13S,14S,17S)-3-hydroxy-3,10,13-trimethylhexadecahydro-1H-cyclopenta[a]phenanthren-17-yl)ethan-1-one O-(cyclobutyl(2-(methylamino)ethyl)carbamoyl) oxime hydrochloride (20c)

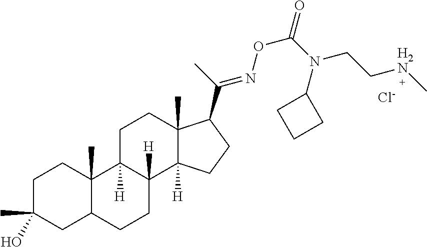

Prepared according to General Procedure F using 19c as the starting Boc-protected analog. The product was in the form of a white solid (45%). ¹H NMR (600 MHz, CDCl₃) δ 9.48 (s, 2H), 4.36 (s, 1H), 3.80 (q, J=6.6 Hz, 2H), 3.16 (s, 2H), 2.76 (s, 3H), 2.35 (t, J=9.1 Hz, 1H), 2.27-2.13 (m, 5H), 1.99 (s, 3H), 1.91-1.79 (m, 1H), 1.78-1.56 (m, 6H), 1.54-1.41 (m, 5H), 1.40 (s, 3H), 1.32-1.09 (m, 12H), 1.03-0.88 (m, 1H), 0.85-0.78 (m, 1H), 0.75 (s, 3H), 0.66 (s, 3H). ¹³C NMR (151 MHz, CDCl₃) δ 166.5, 157.6, 69.8, 56.9, 56.0, 54.1, 44.4, 41.7, 41.0, 38.8, 35.6, 35.5, 34.8, 34.0, 33.4, 31.8, 31.5, 29.2, 28.3, 24.1, 23.0, 20.9, 17.4, 14.6, 13.6, 11.2.

### H. Preparation of (E)-1-((3R,8R,9S,10S,13S,14S,17S)-3-hydroxy-3,10,13-trimethylhexadecahydro-1H-cyclopenta[a]phenanthren-17-yl)ethan-1-one O-(isopropyl(2-(methylamino)ethyl)carbamoyl) oxime hydrochloride (20d)

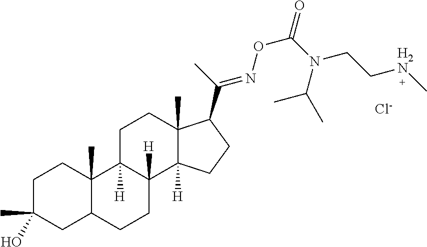

Prepared according to General Procedure F using 19d as the starting Boc-protected analog. The product was in the form of a white solid (47%). ¹H NMR (600 MHz, CDCl₃) δ 9.37 (d, J=112.6 Hz, 2H), 4.25 (s, 1H), 3.69 (d, J=14.2 Hz, 1H), 3.56-3.42 (m, 1H), 3.25 (d, J=21.6 Hz, 2H), 2.71 (d, J=5.1 Hz, 3H), 2.38 (t, J=9.1 Hz, 1H), 2.29 (s, 2H), 2.08-1.99 (m, 1H), 1.96 (s, 3H), 1.83 (dd, J=8.4, 2.7 Hz, 1H), 1.76-1.65 (m, 3H), 1.63-1.46 (m, 5H), 1.43-1.32 (m, 2H), 1.32-1.09 (m, 17H), 0.97 (qd, J=12.5, 4.7 Hz, 1H), 0.80 (dt, J=11.0, 5.7 Hz, 1H), 0.76 (s, 3H), 0.66 (s, 3H). ¹³C NMR (151 MHz, CDCl₃) δ 167.5, 157.3, 69.8, 56.3, 55.7, 54.1, 48.3, 44.7, 41.8, 41.1, 39.2, 38.5, 35.6, 34.9, 34.1, 33.5, 31.8, 31.6, 29.7, 28.4, 24.2, 23.2, 20.9, 20.5, 20.5, 16.7, 13.6, 11.2.

### Example 9. Preparation of C20-Oxime Phosphate Prodrugs

C20-oxime phosphate prodrugs were prepared according to Scheme 8.

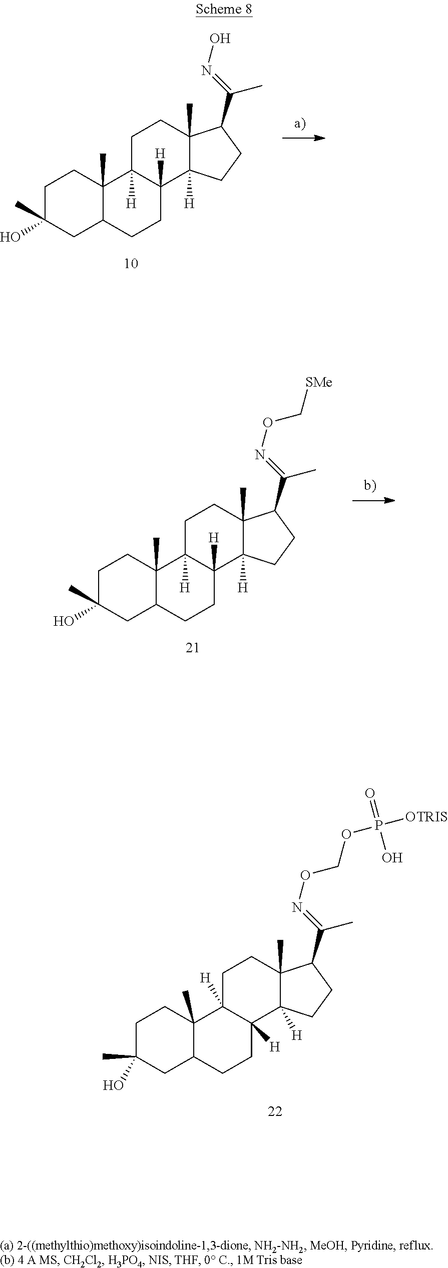

### A. Preparation (E)-1-((3R,8R,9S,10S,13S,14S,17S)-3-hydroxy-3,10,13-trimethylhexadecahydro-1H-cyclopenta[a]phenanthren-17-yl)ethan-1-one O-((methylthio)methyl) oxime (21)

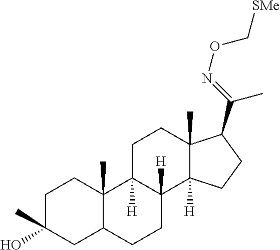

To a stirred mixture of (methylthio)methoxy-1H-isoindole-1,3(2H)-dione (31.6 mmol) in methanol (150 mL) at rt under nitrogen, was added hydrazine monohydrate (29.2 mmol) dropwise via syringe over 2 min. The mixture immediately turned orange, and was stirred for 20 min, after which time a white solid formed suspended in a light-yellow solution. Pyridine (50 mL) was added, followed by 10. A reflux condenser was added to the apparatus, and the mixture was gently refluxed overnight with stirring. After 16 h reflux, the mixture was cooled to room temperature and poured into sat. aq. NH₄Cl (500 mL), and the resulting mixture was extracted with dichloromethane (3×500 mL). The combined organic layers were washed sequentially with water and brine (1×500 mL each), dried over Na₂SO₄, filtered, and concentrated by rotary evaporation to give 24 g crude material. The crude was immobilized on Celite, and automated flash chromatography (330 g column, 1 to 15% EtOAc in DCM gradient) removed bulk impurities to give 8 g semi-purified material. A second automated flash chromatography (220 g column, 1 to 10% EtOAC in DCM gradient) provided 21 (74%) as a white solid.

### B. 1,3-Dihydroxy-2-(hydroxymethyl)propan-2-aminium ((((E)-1-((3R,8R,9S,10S,13S,14S,17S)-3-hydroxy-3,10,13-trimethylhexadecahydro-1H-cyclopenta[a]phenanthren-17-yl)ethylidene)amino)oxy)methyl hydrogen phosphate (22)

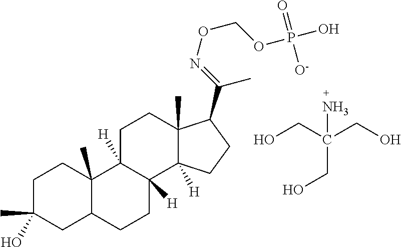

A solid charge of thioether 21 was added to a clean reactor followed by 30 vol (volumes) of 2-methyl tetrahydrofuran (2-Me THF) and 6 g/g of anhydrous 4A molecular sieve beads. The mixture was chilled to −5 to 5° C. and a solid charge 9.4 eq of anhydrous crystalline phosphoric acid was added slowly, followed by addition of 15. eq. (equivalents) of N-iodosuccinimide. A 11-12° C. temperature increase, and a dark yellow/brown suspension was observed. The reaction mixture was stirred at −5 to 5° C. for not more than 40 minutes. The reaction mixture was then vacuum filtered into a chilled, clean reactor at −5 to 5° C., and the filter cake was washed with 10 vol 2-Me THF. The combined filtrates were charged to a clean reactor. A liquid charge of 12° C. pre-cooled aqueous sodium bisulfite (25 vol, 4. w/v %, 4 eq.) was added between 5-15° C. This addition formed an organic top layer and aqueous bottom layer phase. The top organic layer was isolated and washed three times with 5 vol of water at 10-18° C. in a time period of not more than 30 minutes. A liquid charge of 1M TRIS (aqueous) was added to the organic layer to adjust the bottom aqueous layer to a pH between 7.0-8.0 at 9-13 CC. After TRIS charge completion, the mixture was stirred at 5-10° C. for not less than 20 minutes. The aqueous layer was separated and transferred to a chilled, clean reactor at 0-15° C. The organic layer was washed with 2 vol of water at 5-10° C., and the combined aqueous washes were added to the reactor containing the first aqueous layer. The combined aqueous layers were filtered through a celite bed to remove any solid impurities. Purification of the aqueous layer directly loaded on the reverse phase chromatography furnished POM derivatives 22 (48%) as a fluffy white solid. ¹H NMR (600 MHz, CD₃OD) δ ¹H 5.43 (d, J=10.3 Hz, 1H), 3.64 (s, 6H), 2.28-2.10 (m, 2H), 1.84 (s, 3H), 1.74-1.03 (m, 23H), 1.01-0.88 (m, 1H), 0.76 (d, J=1.9 Hz, 4H), 0.62 (s, 3H). ¹³C NMR (151 MHz, CD₃OD) δ 161.50, 93.15, 70.34, 62.65, 61.09, 58.08, 57.40, 55.84, 45.16, 42.69, 42.08, 40.19, 37.14, 36.68, 35.52, 35.20, 33.29, 31.81, 29.67, 25.23, 24.04, 22.11, 16.61, 13.89, 11.68.

### Example 10. Preparation of Additional Neurosteroid Analogs

The synthesis of neurosteroid 29 is shown in Scheme 9 below. Oxidation of the commercially available nandralone (23) to the ketone containing compound 24 was accomplished by use of chromium(vi) oxide with acetic acid as solvent, as outlined in Brosa, et al., Journal of Organic Chemistry, 1989, 54, 3984-3985. With 25 in hand, the synthesis of 29 followed the literature (Botella et al., Journal of Medicinal Chemistry, 2017, 60, 7810-7819 and WO2015/180679). Firstly, reduction of the internal alkene group of 24 was accomplished by use of Pd/C and hydrogen gas with hydrobromic acid as additive. This afforded compound 25, which was then subjected to nucleophilic attack by MeMgBr at the C3 carbon, to provide the methyl and hydroxyl moieties. To ensure the correct stereochemistry, the bulky methylaluminum reagent MAD was implemented (Maruoka et al., Journal of American Chemistry Society, 1985, 107, 4573-7576). This provided compound 26 in good yield. Next, a Wittig reaction provided the methyl alkene derivative at the C20 position found in compound 27, which was then subjected to hydroboration oxidation reaction sequence utilizing either BH₃·Me₂S in THF. This afforded the penultimate compound 28 which was carried forward without purification to undergo oxidation with PCC to afford the desired compound 29 with ketone handle at C20. ¹H and ¹³C NMR spectra of all compounds corresponded to the literature listed above.

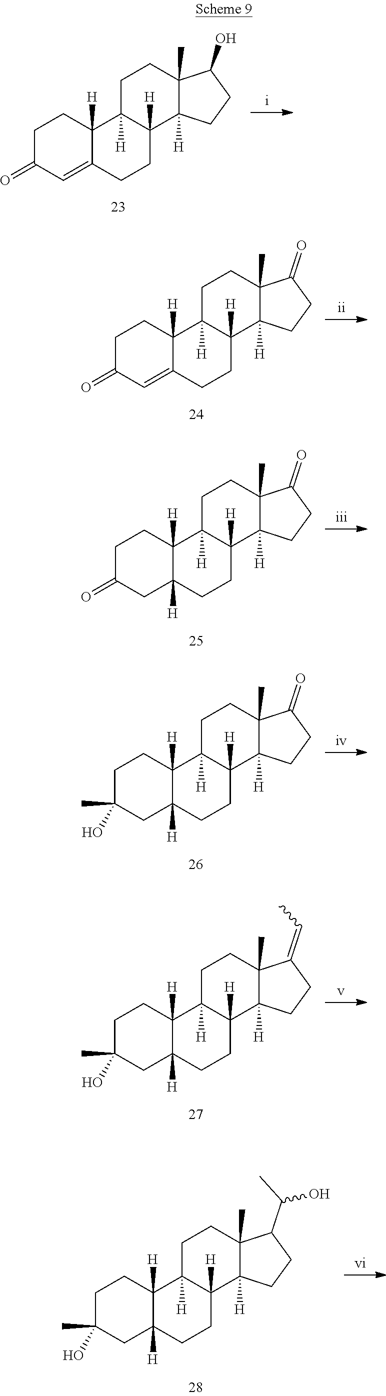

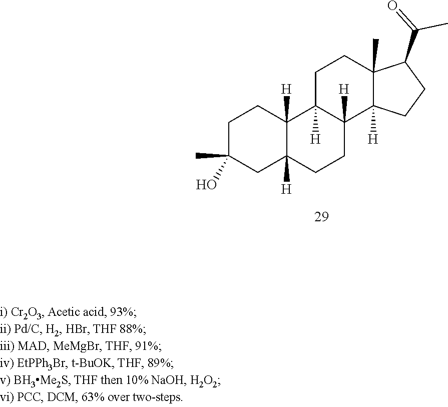

### Example 11. Preparation of Additional Neurosteroid C20-Oxime Compounds

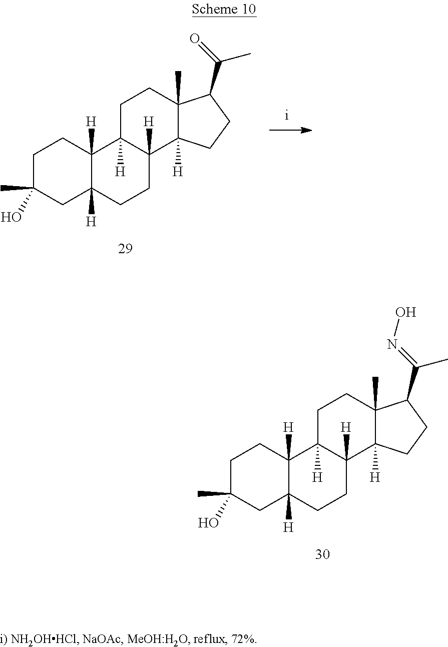

The synthesis of the oxime 30 ((E)-1-((3R,5R,8R,9R,10S,13S,14S,17S)-3-hydroxy-3,13-dimethylhexadecahydro-1H-cyclopenta[a]phenanthren-17-yl)ethan-1-one oxime) is shown in Scheme 10 above. With the C20 ketone handle of 29 in hand, reaction with hydroxylamine hydrochloride and sodium acetate refluxing in methanol provided the C20 oxime analog 30.

To a Schenck flask with a stirrer bar, and condenser was added neurosteroid 29 (100 mg, 0.314 mmol, 1 equiv.) and methanol (10 mL/mmol). A separate solution containing sodium acetate (57 mg, 0.691 mmol, 2.2 equiv.), hydroxylamine hydrochloride (44 mg, 0.628 mmol, 2 equiv.), and water (4 mL/mmol) was then slowly added dropwise. After addition, the mixture was allowed to reflux until complete conversion as observed by TLC (3 h). A thick white precipitate formed during the course of the reaction. Afterwards, the reaction was quenched with 20 mL water and extracted with 30 mL ethyl acetate. The organic layer was separated and washed with saturated ammonium chloride solution and brine. The organic layer was then dried over anhydrous sodium sulfate and concentrated in vacuo and subjected to flash chromatography (DCM:EtOAc) to afford 30 as a white foam (73.3 mg, 0.21 mmol, 70%). ¹H NMR (600 MHz, CDCl₃) δ 1H NMR (600 MHz, CDCl₃) δ 10.33 (s, 1H), 2.23-2.17 (m, 1H), 2.16-2.06 (m, 1H), 1.83-1.77 (m, 1H), 1.75-1.73 (m, 1H), 1.73-1.70 (m, 5H), 1.67-1.51 (m, 4H), 1.42-0.93 (m, 18H), 0.54 (s, 3H). ¹³C NMR (151 MHz, CDCl₃) δ 155.11, 69.72, 56.42, 54.52, 43.41, 41.50, 41.07, 40.07, 38.49, 37.36, 34.32, 34.05, 31.14, 26.26, 25.80, 25.35, 24.98, 23.66, 22.66, 15.13, 13.19. HRMS (ESI+) [M+H]⁺ calc. for C₂₁H₃₅NO₂, 334.27460, observed, 334.27462.

### Example 12. Prophetic Synthesis of C5-Deuterated C20-Oxime Neurosteroid Analogs

The prophetic synthesis of the deuterated neurosteroid oxime 38 is shown in Scheme 11. For the prophetic synthesis of 38, the reaction sequence will follow the same synthesis as outlined in Scheme 6 and 7, with the exception of the first reaction.

This first reaction will use the commercially available nandralone (23) first undergoing an addition reaction with deuterium oxide and two metal catalysts, namely nickel dichloride and zinc, while being heated in dioxane. This will be followed by treatment with sodium hydroxide while heating in water for 24 hours to provide the deuterated analog 31. Following this, the reaction sequence follows the same synthetic methodology laid out in Scheme 6 and 7, which will provide compound 38.

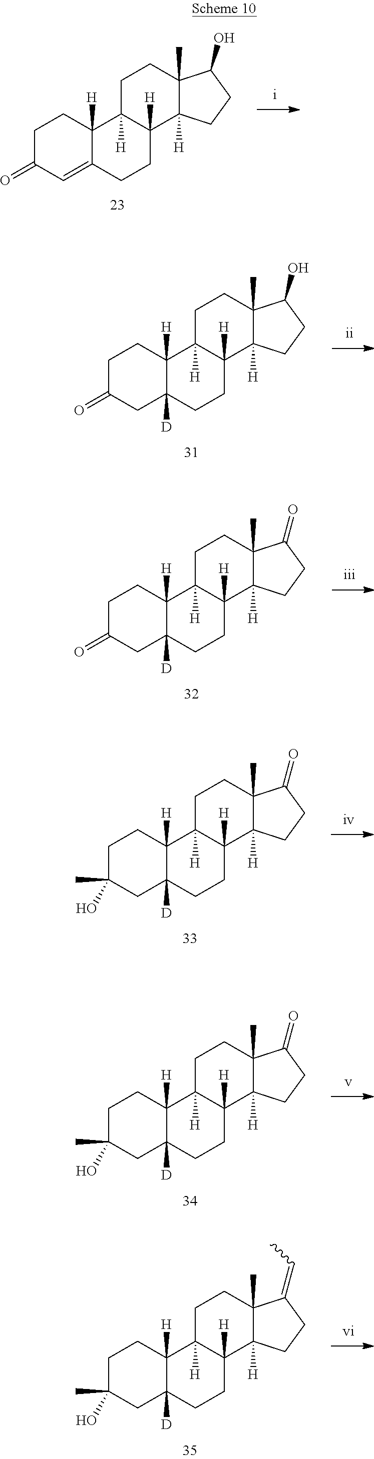

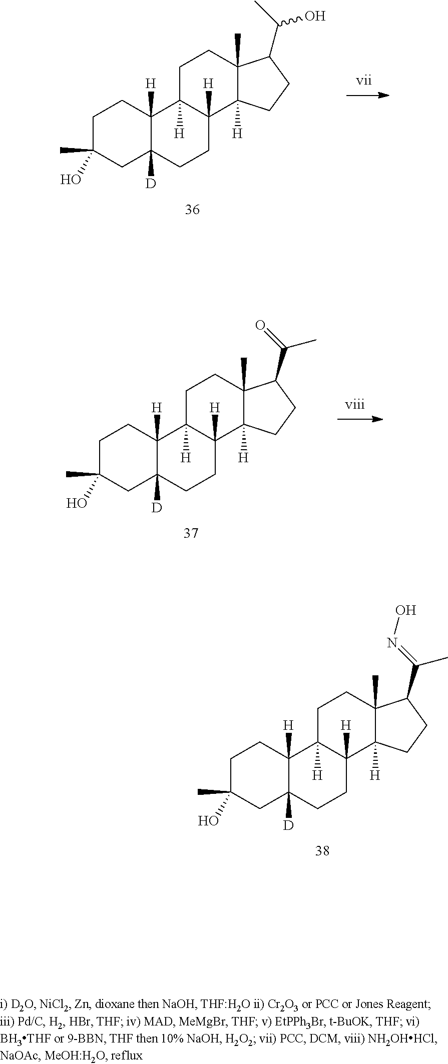

Another method for the prophetic synthesis of the deuterated neurosteroids and C20 oxime 38 is shown in Scheme 12 below. The starting reagent will be the commercially available progesterone (39), which can be subjected to similar reaction conditions outlined in Scheme 8 and Scheme 6. The first reaction will utilize the addition reaction previously discussed with metal catalysts, followed by reaction with sodium hydroxide to afford the intermediate compound 40. Then, nucleophilic attack by MeMgBr at the C3 carbon, will provide the methyl and hydroxyl moieties, with the correct stereochemistry guided by use of MAD. This will provide the desired deuterated neurosteroid core 38.

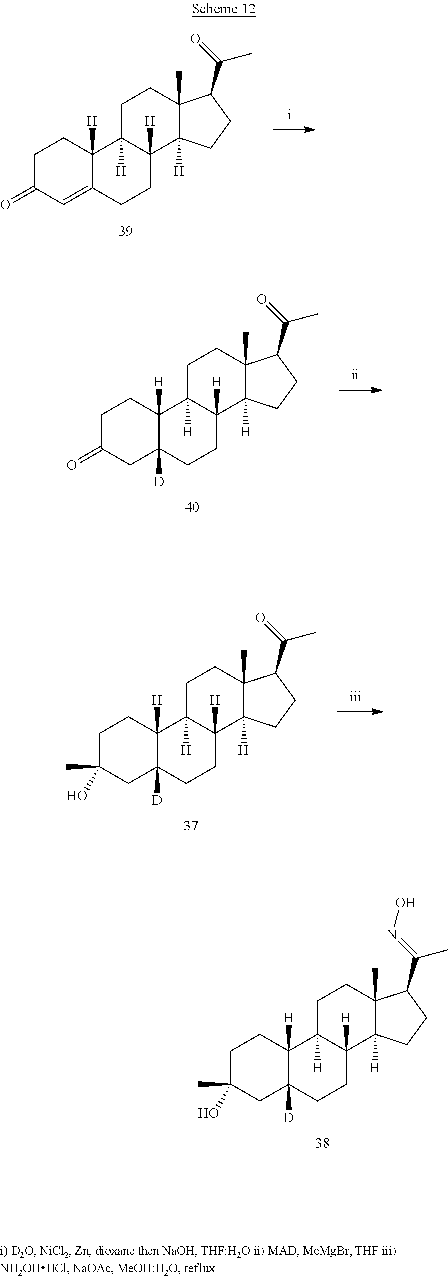

### Example 13. Prophetic Synthesis of Additional C20-Oxime Neurosteroid Prodrugs

The prophetic synthesis of the prodrugs 45a and 45b are shown in Scheme 13 below. From the C20 ketone handle compounds 29, silyl protection of the free hydroxyl and oxime formation will afford compound 42. Conversion to the activated electrophile 43 will then be accomplished by reaction of 42 with 4-nitrophenylchloroformate. With the key reactive intermediate 43, reaction with the previously synthesized and purchased building blocks tert-butyl methyl(2-(methylamino)ethyl)carbamate hydrochloride and tert-butyl (S)-2-((methylamino)methyl)pyrrolidine-1-carboxylate will afford the boc protected analogs 44a-b respectively. Finally, simultaneous deprotection of the silyl and boc protected groups will afford the final prodrugs 45a-b as the hydrochloride salts. It is important to note that other amine nucleophiles represented may react with 43 to generate prodrugs in this manner. Also, deuterated or halogenated analogs of these prodrugs can be made in a similar manner.

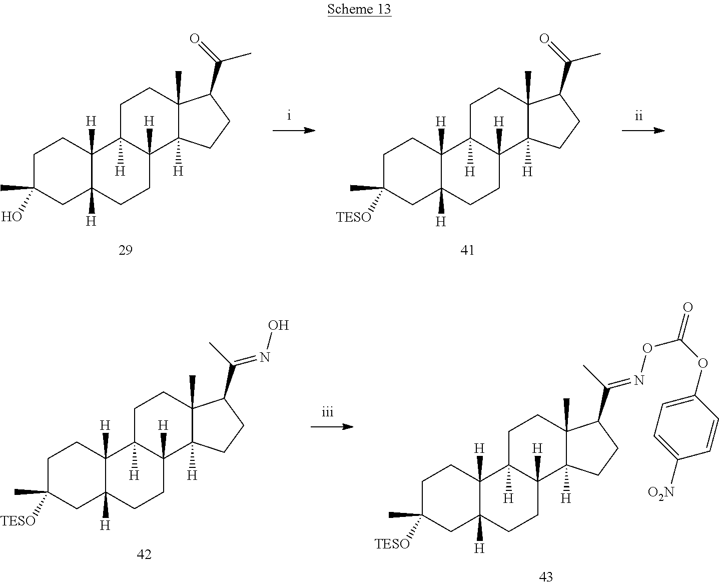

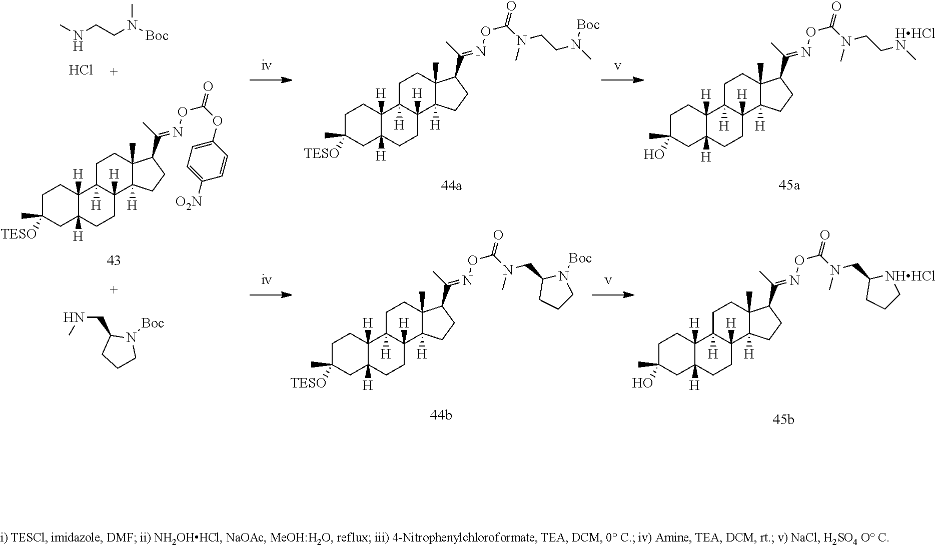

### Example 14. Prophetic Synthesis of Additional C20-Oxime Phosphate Prodrugs

The prophetic synthesis of the prodrug 47 is shown in Scheme 14 below. This will be accomplished in a similar manner for that of compound 22 in Scheme 8. Also, deuterated or halogenated analogs of this prodrug can be made in a similar manner.

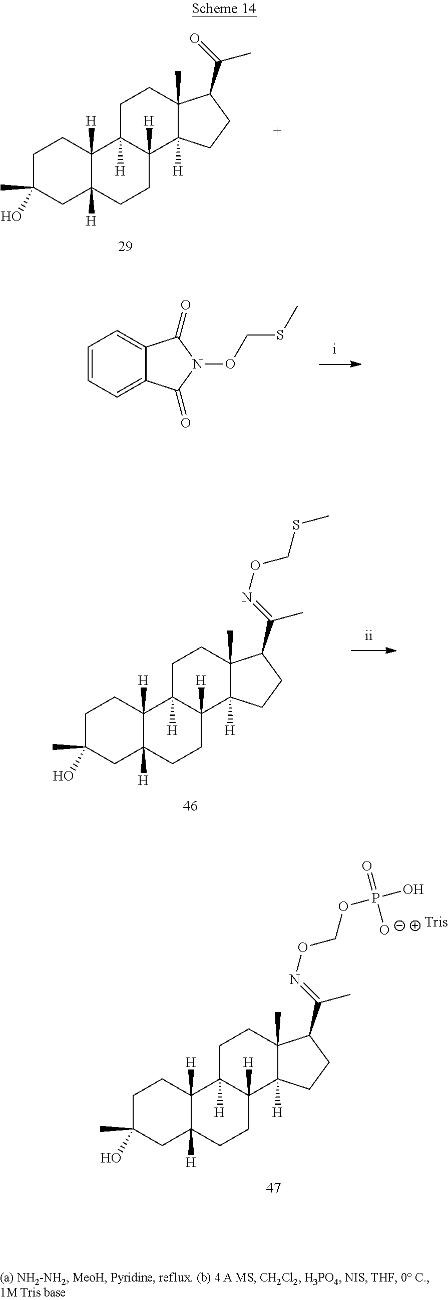

### Example 15. Prophetic Synthesis of Additional C20-Oxime Neurosteroid Analogs

The prophetic synthesis of the oxime 51 is shown in Scheme 15 below. With the C20 ketone handle of 29 in hand, reaction with bromine under acidic conditions will afford the brominated analogs 49. Oxime formation can then be undertaken by reaction of 49 with hydroxylamine hydrochloride to afford compound 50. Lastly, the heterocyclic nucleophile 1H-pyrazole-4-carbonitrile can be reacted with 50 to afford the desired heterocyclic containing oxime compound 51.

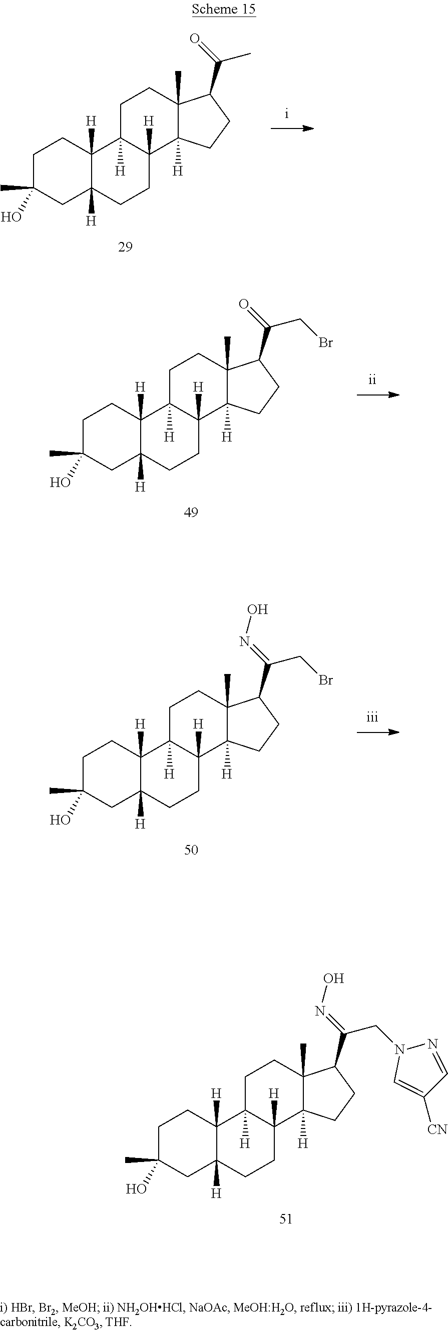

Alternatively, compound 51 can also be synthesized as outlined in Scheme 16 below.

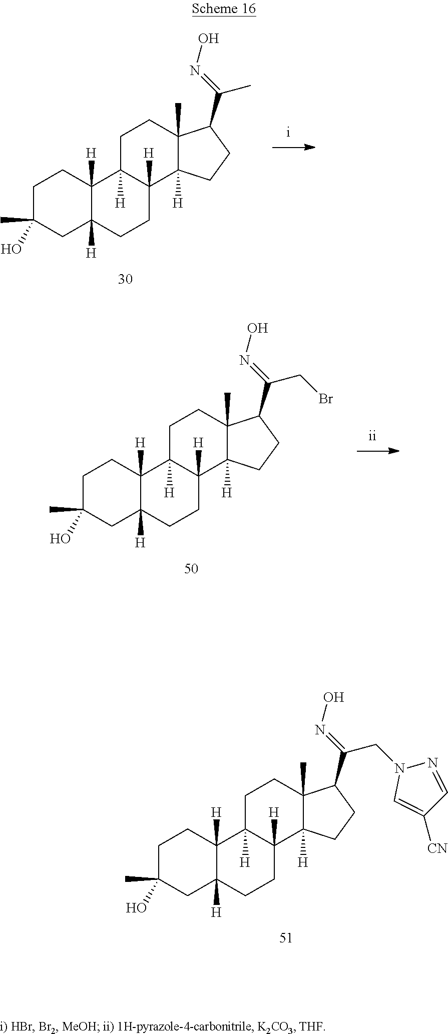

C20-oxime neurosteroid analogs containing a different nitrogen-containing 5-membered heteroaryl, such as those disclosed in Section II(A), can be synthesized using similar methods. Their corresponding phosphate prodrugs and non-phosphate prodrugs, as described herein, can be synthesized using methods described in other examples such as Examples 13 and 14.

### Example 16. Bioanalytical Studies

### A. Materials and Methods

### Microsomal Stability Assay

3 μM test compound was incubated in Human Liver Microsomes (Xenotech) at 37° C. with aliquots (100 L) removed at 0, 5, 10, 15, and 30 minutes. Enzymatic activity was quenched at each time point with the addition of 200 μL of acetonitrile containing 2 μM internal standard (d5-7-ethoxy coumarin), immediately vortexed and centrifuged at 15,000 rpm for 25 min at 4° C. Supernatants were transferred to autosampler vials for LC-MS/MS analysis. LC-MS/MS analysis was performed using Agilent Infinity II 1260 HPLC coupled to a G6460C QQQ MS. Half-lives were determined by plotting ln of % test compound remaining versus time and performing linear regression to determine the slope. Slope=−k and t½=0.693/k for first order kinetics.

### Plasma Stability Assay

3 μM test compound was incubated in Human or Mouse plasma (BioIVT) at 37° C. with aliquots (100 μL) removed at 0, 15, 30, 60, 120, and 240 minutes. Enzymatic activity was quenched at each time point with the addition of 200 μL of acetonitrile containing 2 μM internal standard (d5-7-ethoxy coumarin), immediately vortexed and centrifuged at 15,000 rpm for 25 min at 4° C. Supernatants were transferred to autosampler vials for LC-MS/MS analysis. LC-MS/MS analysis was performed using Agilent Infinity II 1260 HPLC coupled to a G6460C QQQ MS. Half-lives were determined by plotting ln of % test compound remaining versus time and performing linear regression to determine the slope. Slope=−k and t½=0.693/k for first order kinetics.

### Aqueous Stability Assay

Aqueous stability experiments were performed on an Agilent 1100 HPLC equipped with an Agilent 1200 autosampler. HPLC grade solvents were exclusively used during analysis, with acetonitrile being purchased from Sigma Aldrich, and water and formic acid provided by Fisher Scientific. Sample solutions were prepared in 2.0 mL large opening RAM™ vials from Chemglass using DPBS as the aqueous medium. Filtration of undissolved material was achieved with Whatman Puradisc 4 mm PTFE (0.45 μm) syringe filters from Cytiva.

Tested compounds were dissolved in DPBS at a standard concentration of 1 mg/mL. All mixtures were then sonicated for 5 min or stirred until complete dissolution was achieved. Any precipitate observed in sample solutions was filtered with a PTFE syringe filter prior to analysis. Immediately after sample preparation, injection by the HPLC autosampler was performed, which was recorded as the initial time point (t=0 min). Samples were then kept in the autosampler at room temperature and injected at designated intervals without additional filtration or sample modification. Next, the area of the parent peak in each chromatogram was determined by automated integration in the Agilent ChemStation software. Pseudo first-order half-lives were then ascertained by calculating the percent of the parent compound remaining after each time point, Plots of time (x-axis) vs. the natural logarithm of percent parent remaining (y-axis) were subsequently constructed to determine the slope. Finally, the aqueous stability was evaluated using the following equation:

$${t}_{1 / 2} = ln ( 2 ) / - slope$$

The HPLC conditions were as follows: injection volume, 1 μL; column temperature: 40° C.; detector: 254 nm; stationary phase: Zorbax Eclipse XDB-C18 (4.6×150 mm, 5 μm); flow rate: 1 mL/min; mobile phase: 80-95% acetonitrile in H₂O (0.1% formic acid).

### B. Results

The stability data for C20-oxime compounds are shown in Table 1 below. The prodrugs exhibit high aqueous solubility with varying degrees of stability in PBS and human plasma.

TABLE 1
Bioanalytical characterization of C20-oxime neurosteroids and prodrugs thereof

| | | | | Human | |
|---|---|---|---|---|---|
| | | PBS | HLM | plasma | Aqueous |
| Compound | | (% REM | t_{1/2} | t_{1/2} | solubility |
| name | Structure | at 2 hr) | (min) | (min) | (μM) |
| GP-GAN- 001 (10) | 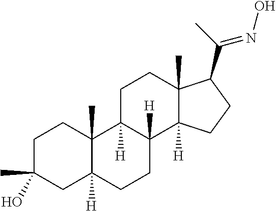 | N/A | >2 hr | >4 hr | N/A |
| GP-GAN- 006 (15) | 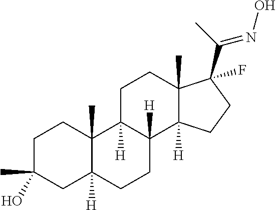 | 61 | N/A | 3 | N/A |
| LEH- NEURO- 007 (30) | 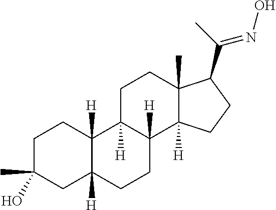 | N/A | N/A | >4 hr | N/A |
| GP-GAN- 002 (20a) | 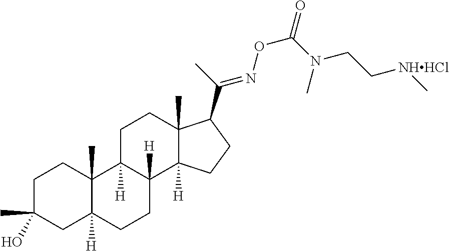 | 66 | N/A | 55 | >750 |
| GP-GAN- 003 (20b) | 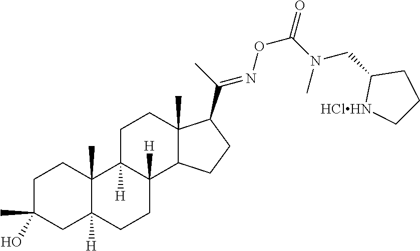 | 10 | N/A | 6 | >750 |
| GP-GAN- 005 (20c) | 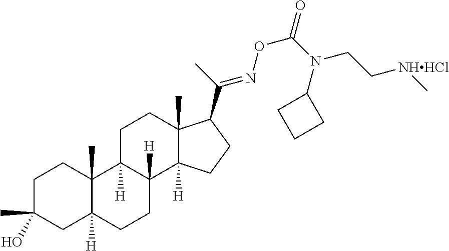 | 43 | N/A | 39 | >750 |
| GP-GAN- 004 (20d) | 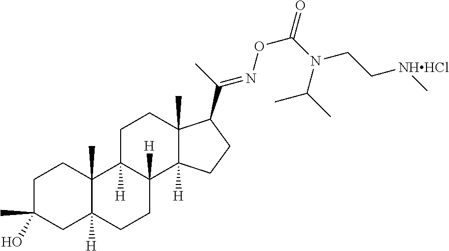 | 52 | N/A | 28 | >750 |
| GP-GAN- 007 (22) | 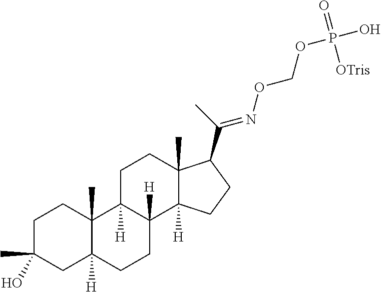 | N/A | N/A | N/A | >1000 |

N/A: not available

### Example 17. In Vivo Pharmacokinetic Studies

### A. Materials and Methods

In vivo pharmacokinetic studies of prodrug 22 were performed in C57BL/6 mice. The plasma distribution profiles and brain distribution profiles of the parent compound 10 were determined following either a single IV administration or a single oral administration of prodrug 22 at 10 mg/kg and 20 mg/kg equivalent dose to 10, respectively. For IV dose, plasma and brain sampling was performed in triplicate at 0.08, 0.25, 0.5, 1, 2, 4, 8, and 12 hr timepoints. For oral dosing, plasma and brain sampling was performed in triplicate at 0.25, 0.5, 1, 2, 4, 8, and 12 hr timepoints. Compound 22 was formulated in 20% HP-β-CD in normal saline. IV dose for compound 22 was 16.7 mg/kg and oral dose was 33.4 mg/kg. Presence of compound 10 was monitored in both plasma and brain compartments using LC-MS.

### B. Results

IV pharmacokinetic result of compound 22 (quantification of the parent compound 10) is shown in FIG. 2, with representative pharmacokinetic parameters summarized in Table 2.

TABLE 2
IV pharmacokinetic parameters of compound 22
(quantification of the parent compound 10)

| | AUC (hr*ng/mL) | T_{1/2} (hr) | Tₘₐₓ | Cₘₐₓ (ng/mL) |
|---|---|---|---|---|
| Plasma | 1222 | 2.04 | 5 min | 3420 |
| Brain | 2033 | 2.32 | 15 min | 2200 |

AUC_{IV brain}/AUC_{IV plasma}: 1.65

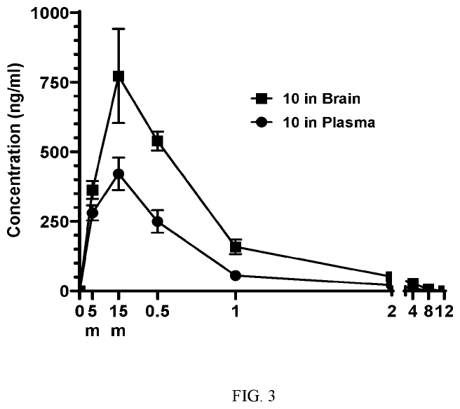

Oral pharmacokinetic result of compound 22 (quantification of the parent compound 10) is shown in FIG. 3, with representative pharmacokinetic parameters summarized in Table 3.

TABLE 3
Oral pharmacokinetic parameters of compound
22 (quantification of the parent compound 10)

| | AUC (hr*ng/mL) | T_{1/2} (hr) | Tₘₐₓ | Cₘₐₓ (ng/mL) |
|---|---|---|---|---|
| Plasma | 850 | 2.54 | 30 min | 421 |
| Brain | 1904 | 2.73 | 30 min | 773 |

AUC_{PO brain}/AUC_{PO plasma}: 2.23
AUC_{PO plasma}/AUC_{2×IV plasma} = 34.7%; AUC_{PO brain}/AUC_{2×IV brain} = 46.8%

### Example 18. GABA_{A} Electrophysiology Profiling

### A. Materials and Methods

Assessment of C20-oxime neurosteroids 10 and 30 and a control compound (ganaxalone) at 6 concentrations against GABA_{A} α4β3δ and α1β2γ2 in PAM (positive allosteric modulator) mode, using the SyncroPatch automated platform at room temperature was completed. The SyncroPatch assay is performed at room temperature. A fast application protocol is used, in which the ligand is rapidly applied and then removed from the cell. To test for PAM mode, the agonist (GABA EC20) is applied three times as a control and to show reproducibility, followed by a minimum 2-minute pre-incubation of client compound and then re-application of agonist (GABA EC20) in the presence of client compound. Finally, a saturating concentration of agonist is applied. A steady-state voltage pulse at −80 mV is applied throughout the assay. Compounds are prepared as 10 mM DMSO stocks.

Automated patch clamp-recordings are performed using the SyncroPatch 384i. The voltage protocol generation and data collection are performed with Patch Controller 384 V2.2.1 and Data Controller V2.3.0. The PAM activity is calculated using the following equation, [(Icomp/Ireference)−1], where Icomp is the current amplitude in the presence of the compound and Ireference is the current amplitude in the presence of GABA alone. This builds an activation concentration-response curve (CRC), where <0.2 represents no PAM activity and >0.2 represents PAM activity. If a “bell-shape curve” is observed at the higher concentrations, showing a reduction in activity in comparison to previous concentrations, then to allow for a more accurate curve fit, these concentrations are removed from the graphs where necessary and noted in the results table.

### B. Results

Table 4 shows that the two C20-oxime compounds 10 and 30 are potentiators of GABA_{A} receptors, similar to ganaxalone. Moreover, both compounds 10 and 30 selectively potentiate a GABA_{A} receptor containing the delta (δ) subunit, compared to a GABA_{A} receptor without the delta (δ) subunit. This selectivity is not observed for ganaxalone.

| | | | GABA_{A} | | GABA_{A} |
|---|---|---|---|---|---|
| | | GABA_{A} | α4β3δ | GABA_{A} | α1β2γ2 |
| | | α4β3δ | Maximum | α1β2γ2 | Maximum |
| Compound | | EC₅₀ | Potentiation | EC₅₀ | Potentiation |
| name | Structure | (nM) | (%) | (nM) | (%) |
| GP-GAN- 001 (10) | 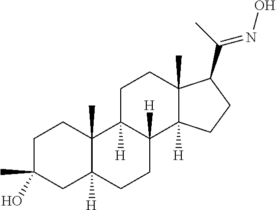 | 1000 | 533 | 1620 | 133 |
| LEH- NEURO- 007 (30) | 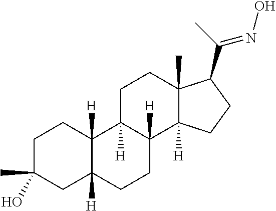 | 383 | 510 | 692 | 121 |
| Ganaxalone | 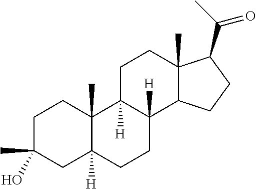 | 879 | 165 | 540 | 252 |

What is claimed is:

1. A compound having a structure of Formula I or a pharmaceutically acceptable salt, hydrate, or hydrated salt thereof: 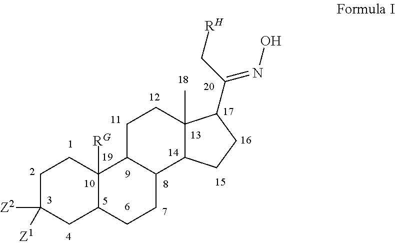
wherein R^{G} is hydrogen or methyl;
wherein R^{H} is hydrogen or an optionally substituted nitrogen-containing 5-membered heteroaryl;
wherein Z^{t} is —OR^{X}, wherein R^{X} is selected from hydrogen, acyl, ester, thioester, and amide; and
wherein Z² is methyl, halogen-substituted methyl, methoxymethyl, or halogen-substituted methoxymethyl.

2. The compound of claim 1, wherein the compound has a structure of Formula I-1 or a pharmaceutically acceptable salt, hydrate, or hydrated salt thereof: 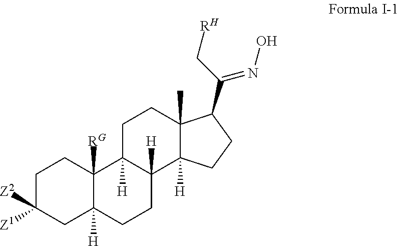
wherein Z¹, Z², R^{G}, and R^{H} are the same as described in claim 1.

3. The compound of claim 1, wherein the compound has a structure of Formula I-2 or a pharmaceutically acceptable salt, hydrate, or hydrated salt thereof: 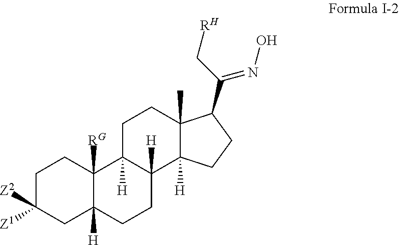
wherein Z¹, Z², R^{G}, and R^{H} are the same as described in claim 1.

4. The compound of claim 1, wherein R^{G} is hydrogen.

5. The compound of claim 1, wherein R^{G} is methyl.

6. The compound of claim 1, wherein R^{X} is H.

7. The compound of claim 1, wherein R^{X} is acyl.

8. The compound of claim 7, wherein R^{X} is —C(═O)R^{e}, wherein R^{e} is C₁-C₆ alkyl, halogen-substituted C₁-C₆ alkyl, C₃-C₆ cycloalkyl, or halogen-substituted C₃-C₆ cycloalkyl.

9. The compound of claim 8, wherein R^{e} is —CF₃.

10. The compound of claim 8, wherein R^{e} is isopropyl.

11. The compound of claim 1, wherein Z² is selected from methyl, —CH₂F, —CHF₂, —CF₃, and —CH₂OCH₃.

12. The compound of claim 11, wherein Z² is methyl.

13. The compound of claim 11, wherein Z² is —CF₃.

14. The compound of claim 11, wherein Z² is —CH₂OCH₃.

15. The compound of claim 1, wherein the compound is selected from: 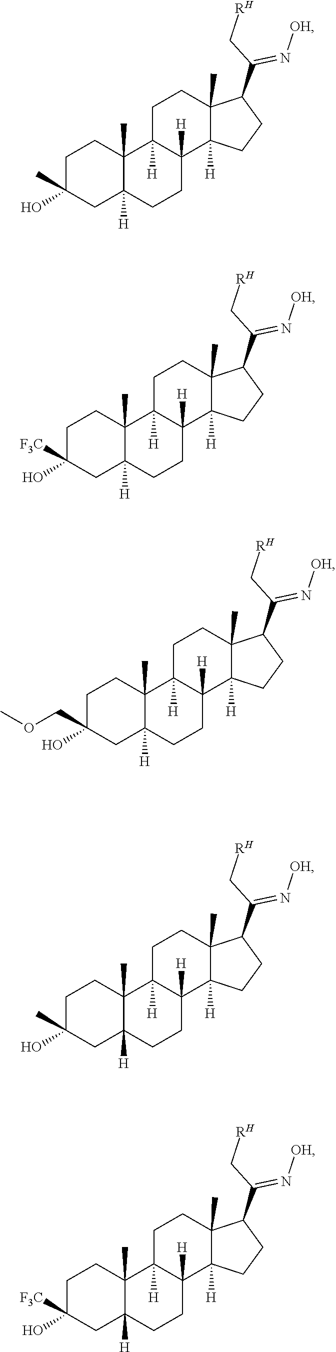 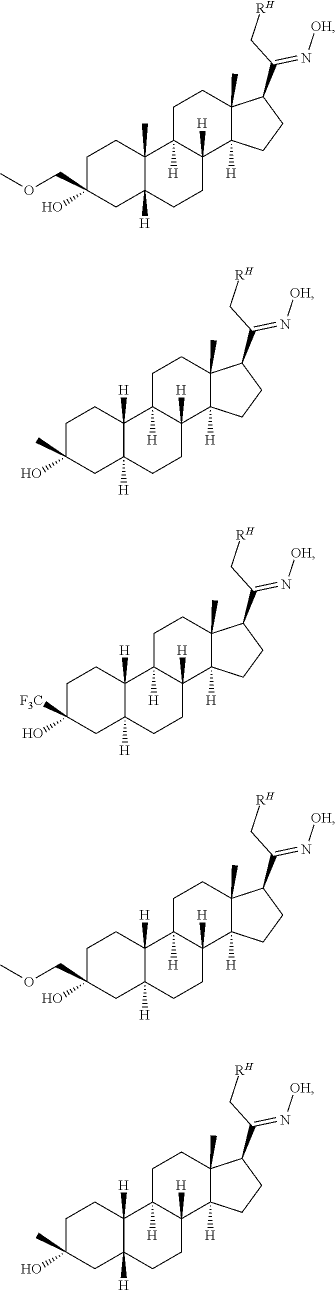 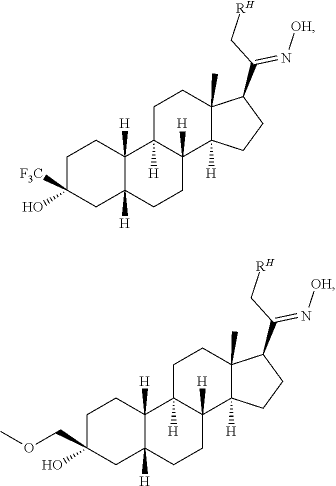
and pharmaceutically acceptable salts, hydrates, or hydrated salts thereof.

16. The compound of claim 1, wherein R^{H} is hydrogen.

17. The compound of claim 1, wherein R^{H} is an optionally substituted nitrogen-containing 5-membered heteroaryl.

18. The compound of claim 17, wherein R^{H} is 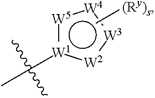
wherein Wt, W², W³, W⁴, and W⁵ are independently and individually C, CH, N, or NH,
wherein at least one of W¹, W², W³, W⁴, and W⁵ is C or CH,
wherein at least one of W¹, W², W³, W⁴, and W⁵ is N or NH,
wherein s is 0, 1, 2, 3, or 4, and
wherein R^{y}, if present, at each occurrence, is independently and individually halogen, nitro, cyano, hydroxyl, formyl, carboxyl, sulfamoyl, alkyl such as methyl, haloalkyl such as CF₃, alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, arylalkyl, alkylaryl, alkyloxy, haloalkyloxy, aryloxy, haloaryloxy, alkylcarbonyl, arylcarbonyl, alkylcarbonyloxy, arylcarbonyloxy, alkyloxycarbonyl, aryloxycarbonyl, primary amino, alkylamino, alkylammonium, alkylcarbonylamino, arylcarbonylamino, carbamoyl, N-alkylcarbamoyl, alkylthio, alkylsulfinyl, alkylsulfonyl, or N-alkylsulfamoyl.

19. The compound of claim 18, wherein W¹ is N.

20. The compound of claim 19, wherein R^{H} is selected from

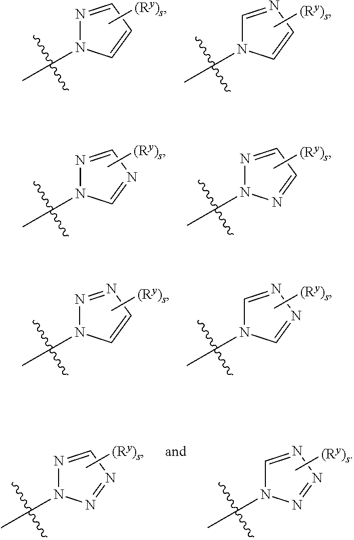

21-173. (canceled)
